(19) United States
(12) Patent Application Publication
(10) Pub. No.: US 2025/0026766 A1
(43) Pub. Date: Jan. 23, 2025

(54) Thiazolopyridyl Amide Derivatives as DNA Polymerase Theta Inhibitors

(71) Applicant: BeiGene, Ltd., Grand Cayman (KY)

(72) Inventors: Jing Li, Beijing (CN); Wenqing Xu, Beijing (CN); Zhiwei Wang, Beijing (CN)

(73) Assignee: BeiGene, Ltd., Grand Cayman (KY)

(21) Appl. No.: 18/761,919

(22) Filed: Jul. 2, 2024

Related U.S. Application Data
(63) Continuation of application No. PCT/CN2023/071799, filed on Jan. 11, 2023.

(30) Foreign Application Priority Data
Jan. 12, 2022 (WO) PCT/CN2022/071563

(51) Int. Cl. C07D 513/04; A61K 31/437; A61K 31/444; A61K 31/496; A61K 31/501; A61K 31/5377; A61K 31/538

(52) U.S. Cl. CPC C07D 513/04; A61K 31/437; A61K 31/444; A61K 31/496; A61K 31/501; A61K 31/5377; A61K 31/538

(57) ABSTRACT

The present invention relates to compounds containing thiazolopyridyl amide structure, the process for their synthesis, as well as the use of such compounds for inhibiting DNA Polymerase Theta (Pol Theta), and in the treatment of various diseases including cancers.

### CROSS-REFERENCE TO RELATED APPLICATIONS

This application is a continuation of International Application No. PCT/CN2023/071799, filed on Jan. 11, 2023, which claims priority to International Application No. PCT/CN2022/071563, filed on Jan. 12, 2022, the disclosure of each of which is hereby incorporated by reference in its entirety.

### FIELD OF THE INVENTION

The present disclosure relates to compounds containing thiazolopyridyl amide structure, the use thereof for inhibiting DNA Polymerase Theta (Pol Theta). The present disclosure also relates to a process for synthesis and to uses of such compounds in the treatment of various diseases including cancers.

### BACKGROUND OF THE INVENTION

Double-stranded nucleic acid (DNA) is the foundation of the genetic information of all living organisms. It constantly encountered damages both endogenously and exogenously. Those damages are classified into various types and repaired by corresponding DNA repair pathways. DNA double-strand break (DSB) is the most lethal damage type due to the potential cell apoptosis and toxic chromosome rearrangement. A comprehensive protein network responsible for the regulation and coordination of DSB repair is called DNA damage response (DDR). Dysfunctional DDR results in inaccurate and invalid DNA repair, thus affecting genomic stability that plays roles in cancer biology (e.g. tumorigenesis, tumor progression, drug-resistance).

Two canonically dominant repair pathways cooperatively repair DSBs in the context of the cell cycle and DNA-end-resection longevity: 1) homologous recombination (HR) that is active in the S/G2 phase and requires lone single-strand overhang, and 2) classic non-homologous end joining (c-NHEJ) that is particularly active in the G1/G2 phase and depend on limited end resection. In addition, another repair pathway, alternative non-homologous end joining (alt-NHEJ), is commonly considered as the backup pathway when the HR or c-NHEJ are compromised [Scully, R., et al., Nat Rev Mol Cell Biol, 2019, 20 (11): p. 698-714].

DNA polymerase theta is the key effector within the alt-NHEJ pathway. DNA polymerase theta is a large protein comprised of N-terminal helicase domain, central domain and C-terminal polymerase domain [Seki, M, et al., Nucleic Acids Rs, 2003, 31 (21), 6117-26]. The function of DNA polymerase theta is to be involved in the DNA synthesis activity, strand annealing, as well as regulation of end processing and HR activity [Acharya, N., et al., Curr Genet, 2020, 66 (4), 635-55; Ceccaldi, R., et al., Nature, 2015, 518 (7538), 258-62; Mateos-Gomez, P. A., et al., Nature, 2015, 518 (7538), 254-7; Newman, J. A., et al., Structure, 2015, 23 (12), 2319-30; Ozdemir, A. Y., et al., J Biol Chem, 2018, 293 (14), 5259-69]. DNA polymerase theta is widely overexpressed in various cancer types, associated with resistance to DNA-damaging therapy and poor survival outcome in breast cancer [Higgins, G. S., et al., Oncotarget, 2010, 1 (3), 175-84]. POLQ gene (DNA polymerase theta gene) is identified as the synthetic lethality of BRCA genes and other genes involved in HR pathway [Ceccaldi, R., et al., Nature, 2015, 518 (7538), 258-62; Feng, W., et al., Nat Commun, 2019, 10 (1), 4286; Mateos-Gomez, P. A., et al., Nature, 2015, 518 (7538), 254-7; Mengwasser, et al., Mol Cell, 2019, 73 (5), 885-99].

Targeting DNA repair is the effective and well-proven strategy for cancer treatment, based on the hypothesis that cancer cells rely on specific DNA repair pathways to settle down the excessive genetic stress and promote cell survival. The synthetic lethality of Poly (ADP-ribose) polymerase (PARP) inhibitor is exemplified in BRCA-deficient cancers subtypes. Therefore, DNA polymerase theta is a promising target for novel synthetic lethal therapy in cancer types with defective DSB repair pathway.

### SUMMARY OF THE INVENTION

One objective of the present invention is to provide compounds and derivatives thereof useful as Pol Theta inhibitors.

Aspect 1. The compound of formula (I):

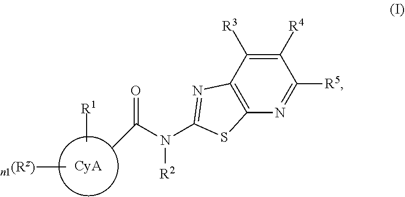

or a N-oxide thereof, or a pharmaceutically acceptable salt thereof, or a stereoisomer thereof, or a tautomer thereof, or a deuterated analog thereof, or a prodrug thereof,
wherein:
ring CyA is a 5- to 12-membered heteroaromatic ring comprising 1 to 4 heteroatoms selected from N, O, or S as ring member(s);
R¹ is —C₁₋₈alkyl, —C₂₋₈alkenyl, —C₂₋₈alkynyl, cycloalkyl, haloalkyl, heterocyclyl, aryl, heteroaryl, oxo, —CN, —OR^{1a}, —SO₂R^{1a}, —SO₂NR^{1a}R^{1b}, P(O)R^{1a}R^{1b}, —COR^{1a}, —CO₂R^{1a}, —CONR^{1a}R^{1b}, —NR^{1a}R^{1b}, —NR^{1a}COR^{1b}, —NR^{1a}CO₂R^{1b}, —NR^{1a}CONR^{1b}R^{1c}, or —NR^{1a}SO₂R^{1b}; wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{1d};
R^{1a}, R^{1b} and R^{1c} are each independently selected from hydrogen, —C₁₋₈alkyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{1f}; or
(R^{1a} and R^{1b}), (R^{1b} and R^{1c}) or (R^{1a} and R^{1c}), together with the atom(s) to which they are attached, form a 3- to 12-membered ring, said ring comprising 0, 1 or 2 heteroatom(s) independently selected from nitrogen, oxygen, or optionally oxidized sulfur as ring member(s), said ring is optionally substituted with at least one substituent R^{1f};
R^{1d} and R^{1f} are each independently selected from hydrogen, halogen, —C₁₋₈alkyl, —C₂₋₈alkenyl, —C₂₋₈alkynyl, cycloalkyl, heterocyclyl, aryl, heteroaryl, oxo, —CN, —OR^{1g}, —SO₂R^{1g}, —SO₂NR^{1g}R^{1h}, —P(O)R^{1g}R^{1h}, —COR^{1g}, —CO₂R^{1g}, —CONR^{1g}R^{1h}, —NO₂, —NR^{1g}R^{1h}, —NR^{1g}COR^{1h}, —NR^{1g}CO₂R^{1h}, —NR^{1g}CONR^{1h}R¹ⁱ, or —NR¹SO₂R^{1h}; wherein each of said —C₁₋₈alkyl, —C₂₋₈alkenyl, —C₂₋₈alkynyl, cycloalkyl, heterocyclyl, aryl or heteroaryl is optionally substituted with at least one substituent RJ; or
two R^{1d} or two R^{1f}, together with the atom(s) to which they are attached, form a 3- to 12-membered ring, said ring comprising 0, 1 or 2 heteroatom(s) independently selected from nitrogen, oxygen, or optionally oxidized sulfur as ring member(s), said ring is optionally substituted with at least one substituent R^{1k};
R^{1g}, R^{1h} and R¹ⁱ are each independently selected from hydrogen, —C₁₋₈alkyl, —C₂₋₈alkenyl, —C₂₋₈alkynyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent halogen, hydroxy, —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R^{1j} and R^{1k} are each independently selected from hydrogen, halogen, —C₁₋₈alkyl, —C₂₋₈alkenyl, —C₂₋₈alkynyl, cycloalkyl, heterocyclyl, aryl, heteroaryl, oxo, —CN, —OR^{1l}, —SO₂R^{1l}, —SO₂NR^{1l}R^{1m}, —COR^{1l}, —CO₂R^{1l}, —CONR^{1l}R^{1m}, —NO₂, —NR^{1l}R^{1m}, —NR^{1l}COR^{1m}, —NR^{1l}CO₂R^{1m}, —NR^{1l}CONR^{1m}R¹ⁿ, or —NR^{1l}SO₂R^{1m}; wherein each of said —C₁₋₈alkyl, —C₂₋₈alkenyl, —C₂₋₈alkynyl, cycloalkyl, heterocyclyl, aryl or heteroaryl is optionally substituted with at least one substituent halogen, hydroxy, —C₁₋₈alkyl, -haloC₁₋₈alkyl, —C₁₋₈ alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl
R^{1l}, R^{1m} and Rⁱⁿ are each independently selected from hydrogen, —C₁₋₈alkyl, —C₂₋₈alkenyl, —C₂₋₈alkynyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent halogen, hydroxy, —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R² is hydrogen, —C₁₋₈alkyl, —C₂₋₈alkenyl, —C₂₋₈alkynyl, cycloalkyl, heterocyclyl, aryl or heteroaryl; wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{2a};
R^{2a} is each independently selected from hydrogen, halogen, —C₁₋₈alkyl, —C₂₋₈alkenyl, —C₂₋₈alkynyl, cycloalkyl, heterocyclyl, aryl, heteroaryl, oxo, —CN, —OR^{2b}, —SO₂R^{2b}, —SO₂NR^{2a}R^{2c}, —COR^{2b}, —CO₂R^{2b}, —CONR^{2b}R^{2c}, —NO₂, —NR^{2b}R^{2c}, —NR^{2b}COR^{2c}, —NR^{2b}CO₂R^{2c}, —NR^{2b}CONR^{2c}R^{2d}, or —NR^{2b}SO₂R^{2c}; wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{2e}; or
two R^{2a}, together with the atom(s) to which they are attached, form a 3- to 12-membered ring, said ring comprising 0, 1 or 2 heteroatom(s) independently selected from nitrogen, oxygen, or optionally oxidized sulfur as ring member(s), said ring is optionally substituted with at least one substituent R²¹;
R^{2b}, R^{2c} and R^{2d} are each independently selected from hydrogen, —C₁₋₈alkyl, —C₂₋₈alkenyl, —C₂₋₈alkynyl, cycloalkyl, heterocyclyl, aryl or heteroaryl, wherein each of said —C₁₋₈alkyl, —C₂₋₈alkenyl, —C₂₋₈ alkynyl, cycloalkyl, heterocyclyl, aryl or heteroaryl is optionally substituted with at least one substituent halogen, hydroxy, —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R^{2e} and R^{2f} are each independently selected from hydrogen, halogen, —C₁₋₈alkyl, —C₂₋₈alkenyl, —C₂₋₈alkynyl, —C₁₋₈alkoxy, cycloalkyl, heterocyclyl, aryl, heteroaryl; wherein each of said —C₁₋₈alkyl, —C₂₋₈ alkenyl, —C₂₋₈alkynyl, —C₁₋₈alkoxy, cycloalkyl, heterocyclyl, aryl or heteroaryl is optionally substituted with at least one substituent halogen, hydroxy, —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R³ and R⁴ are each independently hydrogen, halogen, —C₁₋₈alkyl, —C₂₋₈alkenyl, —C₂₋₈alkynyl, cycloalkyl, heterocyclyl, aryl, heteroaryl, oxo, —CN, —OR^{3a}, —SO₂R^{3a}, —SO₂NR^{3a}R^{3b}, —COR^{3a}, —CO₂R^{3a}, —CONR^{3a}R^{3b}, —NR^{3a}R^{3b}, —NR^{3a}COR^{3b}, —NR^{3a}CO₂R^{3b}, —NR^{3a}CONR^{3b}R^{3c}, or —NR^{3a}SO₂R^{3b}; wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{3a};
R^{3a}, R^{3b} and R^{3c} are each independently selected from hydrogen, —C₁₋₈alkyl, —C₂₋₈alkenyl, —C₂₋₈ alkynyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{3f} or
(R^{3a} and R^{3b}), (R^{3b} and R^{3c}) or (R^{3a} and R^{3c}), together with the atom(s) to which they are attached, form a 3- to 12-membered ring, said ring comprising 0, 1 or 2 heteroatom(s) independently selected from nitrogen, oxygen, or optionally oxidized sulfur as ring member(s), said ring is optionally substituted with at least one substituent R^{3f};
R^{3d} and R^{3f} are each independently selected from hydrogen, halogen, —C₁₋₈alkyl, —C₂₋₈alkenyl, —C₂₋₈alkynyl, cycloalkyl, heterocyclyl, aryl, heteroaryl, oxo, —CN, —OR^{3g}, —SO₂R^{3g}, —SO₂NR^{3g}R^{3h}, —COR^{3g}, —CO₂R^{3g}, —CONR^{3g}R^{3h}, —NO₂, —NR^{3g}R^{3h}, —NR^{3g}COR^{3h}, —NR^{3g}CO₂R^{3h}, —NR^{3g}CONR^{3h}R³ⁱ, or NR^{3g}SO₂R^{3h}; wherein each of said —C₁₋₈alkyl, —C₂₋₈alkenyl, —C₂₋₈alkynyl, cycloalkyl, heterocyclyl, aryl or heteroaryl is optionally substituted with at least one substituent halogen, hydroxy, —C₁₋₈alkyl, -haloC₁₋₈alkyl, —C₁₋₈ alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R^{3g}, R^{3h} and R³ⁱ are each independently selected from hydrogen, —C₁₋₈alkyl, —C₂₋₈alkenyl, —C₂₋₈ alkynyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent halogen, hydroxy, —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R⁵ is halogen, —C₁₋₈alkyl, —C₂₋₈alkenyl, —C₂₋₈alkynyl, cycloalkyl, heterocyclyl, aryl, heteroaryl, oxo, —CN, —OR^{5a}, —SR^{5a}, —SO₂R^{5a}, —P(O)R^{5a}R^{5b}, —SO₂NR^{5a}R^{5b}, —COR^{5a}, —CO₂R^{5a}, —CONR^{5a}R^{5b}, —NR^{5a}R^{5b}, —NR^{5a}COR^{5b}, —NR^{5a}CO₂R^{5b}, —NR^{5a}CONR^{5b}R^{5c}, or —NR^{5a}SO₂R^{5b}; wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{5d};
R^{5a}, R^{5b} and R^{5c} are each independently selected from hydrogen, —C₁₋₈alkyl, —C₂₋₈alkenyl, —C₂₋₈ alkynyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{5f}; or
(R^{5a} and R^{1b}), (R^{5b} and R^{5c}) or (R^{5a} and R^{5c}), together with the atom(s) to which they are attached, form a 3- to 12-membered ring, said ring comprising 0, 1 or 2 heteroatom(s) independently selected from nitrogen, oxygen, or optionally oxidized sulfur as ring member(s), said ring is optionally substituted with at least one substituent R^{5f};
R^{5d} and R^{5f} are each independently selected from hydrogen, halogen, —C₁₋₈alkyl, —C₂₋₈alkenyl, —C₂₋₈alkynyl, cycloalkyl, heterocyclyl, aryl, heteroaryl, oxo, —CN, —OR^{5g}, —SO₂R^{5g}, —SO₂NR^{5g}R^{5h}, —COR^{5g}, —CO₂R^{5g}, —CONR^{5g}R^{5h}, —NO₂, —P(O)R^{5g}R^{5h}, —NR^{5g}R^{5h}, —NR^{5g}COR^{5h}, —NR^{5g}CO₂R^{5h}, —NR^{5g}CONR^{5h}R⁵, or —NR^{5g}SO₂R^{5h}; wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{5j}; or
two R^{5d} or two R^{5f}, together with the atom(s) to which they are attached, form a 3- to 12-membered ring, said ring comprising 0, 1 or 2 heteroatom(s) independently selected from nitrogen, oxygen, or optionally oxidized sulfur as ring member(s), said ring is optionally substituted with at least one substituent R^{5k};
R^{5g}, R^{5h} and R⁵ⁱ are each independently selected from hydrogen, —C₁₋₈alkyl, —C₂₋₈alkenyl, —C₂₋₈alkynyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent halogen, hydroxy, —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl; or
(R^{5g} and R^{5h}), (R^{5h} and R⁵ⁱ) or (R^{5g} and R⁵ⁱ), together with the atom(s) to which they are attached, form a 3- to 12-membered ring, said ring comprising 0, 1 or 2 heteroatom(s) independently selected from nitrogen, oxygen, or optionally oxidized sulfur as ring member(s), said ring is optionally substituted with at least one substituent halogen, hydroxy, —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroary;
R^{5j} and R^{5k} are each independently selected from hydrogen, halogen, —C₁₋₈alkyl, —C₂₋₈alkenyl, —C₂₋₈alkynyl, cycloalkyl, heterocyclyl, aryl, heteroaryl, oxo, —CN, —OR^{5l}, —SO₂R^{5l}, —SO₂NR^{5l}R^{5m}, COR^{5l}, —CO₂R^{5l}, —CONR^{5l}R^{5m}, —NO₂, —NR^{5l}R^{5m}, —NR^{5l}COR^{5m}, —NR^{5l}CO₂R^{5m}, —NR^{5l}CONR^{5m}R⁵ⁿ, or —NR^{5l}SO₂R^{5m}; wherein each of said —C₁₋₈alkyl, —C₂₋₈alkenyl, —C₂₋₈alkynyl, cycloalkyl, heterocyclyl, aryl or heteroaryl is optionally substituted with at least one substituent halogen, hydroxy, —C₁₋₈alkyl, -haloC₁₋₈alkyl, —C₁₋₈ alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl
R^{5l}, R^{5m} and R⁵ⁿ are each independently selected from hydrogen, —C₁₋₈alkyl, —C₂₋₈alkenyl, —C₂₋₈alkynyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent halogen, hydroxy, —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R^{z}, at each of its occurrences, is independently hydrogen, halogen, —C₁₋₈alkyl, —C₂₋₈alkenyl, —C₂₋₈alkynyl, cycloalkyl, heterocyclyl, aryl, heteroaryl, oxo, —CN, —OR^{za}, —SO₂R^{za}, —SO₂NR^{za}R^{zb}, —COR^{za}, —CO₂R^{za}, —CONR^{za}R^{zb}, —NR^{za}R^{zb}, —NR^{za}COR^{zb}, —NR^{za}CO₂R^{zb}, —NR^{za}CONR^{zb}R^{zc}, or —NR^{za}SO₂R^{zb}; wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{zd};
R^{za}, R^{zb} and R^{zc} are each independently selected from hydrogen, —C₁₋₈alkyl, —C₂₋₈alkenyl, —C₂₋₈alkynyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{zf}; or
(R^{za} and R^{zb}), (R^{zb} and R^{zc}) or (R^{za} and R^{zc}), together with the atom(s) to which they are attached, form a 3- to 12-membered ring, said ring comprising 0, 1 or 2 heteroatom(s) independently selected from nitrogen, oxygen, or optionally oxidized sulfur as ring member(s), said ring is optionally substituted with at least one substituent R^{zf};
R^{zd} and R^{zf} are each independently selected from hydrogen, halogen, —C₁₋₈alkyl, —C₂₋₈alkenyl, —C₂₋₈ alkynyl, cycloalkyl, heterocyclyl, aryl, heteroaryl, oxo, —CN, —OR^{zg}, —SO₂R^{zg}, —SO₂NR^{zg}R^{zh}, —COR^{zg}, —CO₂R^{zg}, —CONR^{zg}R^{zh}, —NO₂, —NR^{zg}R^{zh}, —NR^{zg}COR^{zh}, —NR^{zg}CO₂R^{zh}, —NR^{zg}CONR^{zh}R^{zi}, or —NR^{zg}SO₂R^{zh}; wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{zj}; or
two R^{zd} or two R^{zf}, together with the atom(s) to which they are attached, form a 3- to 12-membered ring, said ring comprising 0, 1 or 2 heteroatom(s) independently selected from nitrogen, oxygen, or optionally oxidized sulfur as ring member(s), said ring is optionally substituted with at least one substituent R^{zk};
R^{zg}, R^{zh} and R^{zi} are each independently selected from hydrogen, —C₁₋₈alkyl, —C₂₋₈alkenyl, —C₂₋₈alkynyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent halogen, hydroxy, —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R^{zj} and R^{zk} are each independently selected from hydrogen, halogen, —C₁₋₈alkyl, —C₂₋₈alkenyl, —C₂₋₈alkynyl, —C₁₋₈alkoxy, cycloalkyl, heterocyclyl, aryl, heteroaryl, oxo, —CN; wherein each of said —C₁₋₈alkyl, —C₂₋₈alkenyl, —C₂₋₈alkynyl, —C₁₋₈alkoxy, cycloalkyl, heterocyclyl, aryl or heteroaryl is optionally substituted with at least one substituent halogen, hydroxy, —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
n1 is 0, 1, 2, 3, 4, 5, 6, 7 or 8.

Aspect 2. The compound of Aspect 1, wherein the compound is selected from formula (IIa), (IIb), (IIc), (IId) or (IIe):

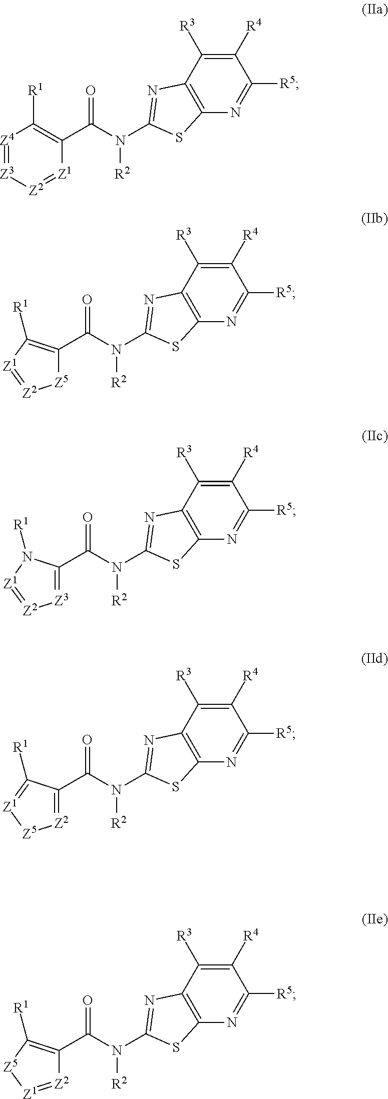

wherein, Z¹, Z², Z³ and Z⁴ are each independently N or CR^{z}; Z⁵ is NR^{z}, O or S;
R¹, R², R³, R⁴ and R⁵ are each defined as claim 1.

Aspect 3. The compound of any one of the preceding aspects, wherein the compound is selected from formula (IIIa), (IIIb), (IIIc), (IIId), (IIIe), (IIIf), (IIIg), (IIIh), (IIIi), (IIIj) or (IIIk):

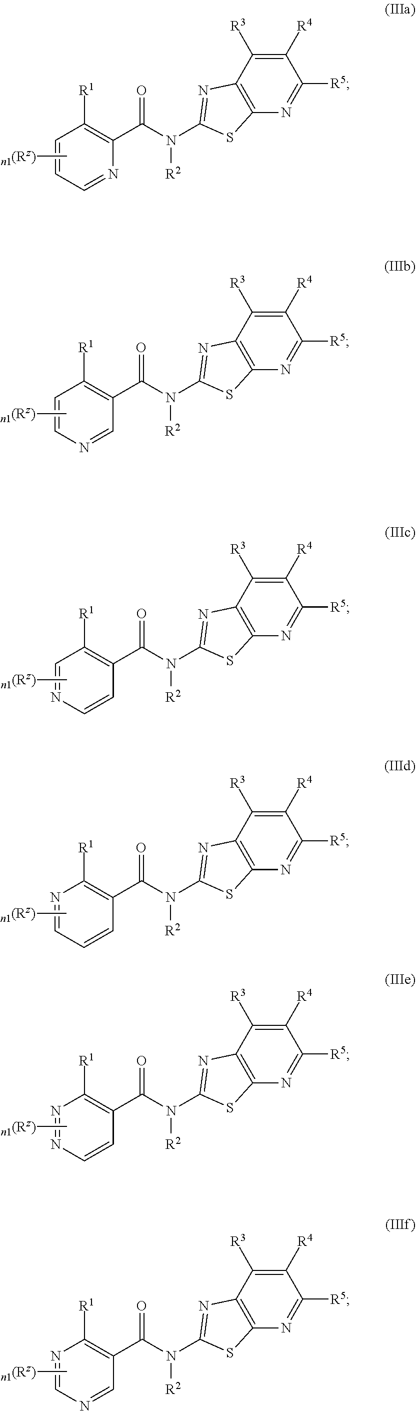

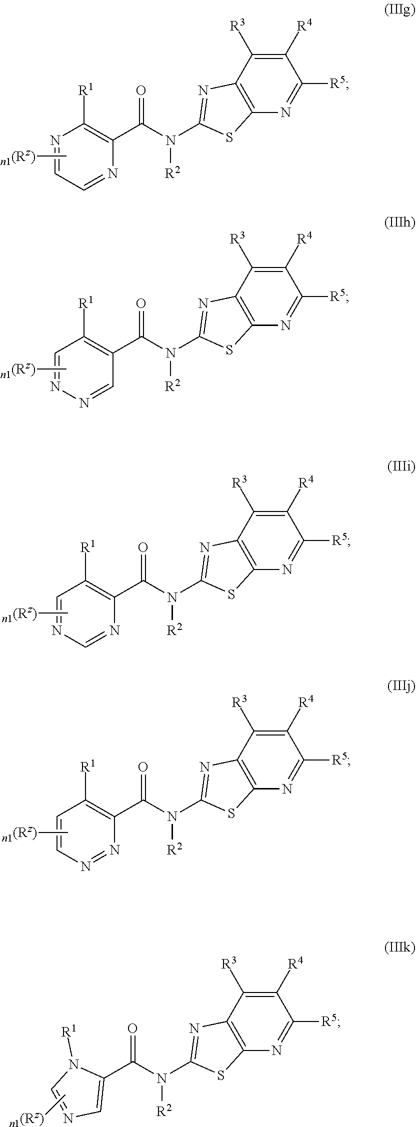

wherein, n1, R^{z}, R¹, R², R³, R⁴ and R⁵ are each defined as claim 1.

Aspect 4. The compound of any one of the preceding aspects, wherein the compound is selected from formula (IVa), (IVb), (IVc) or (IVd):

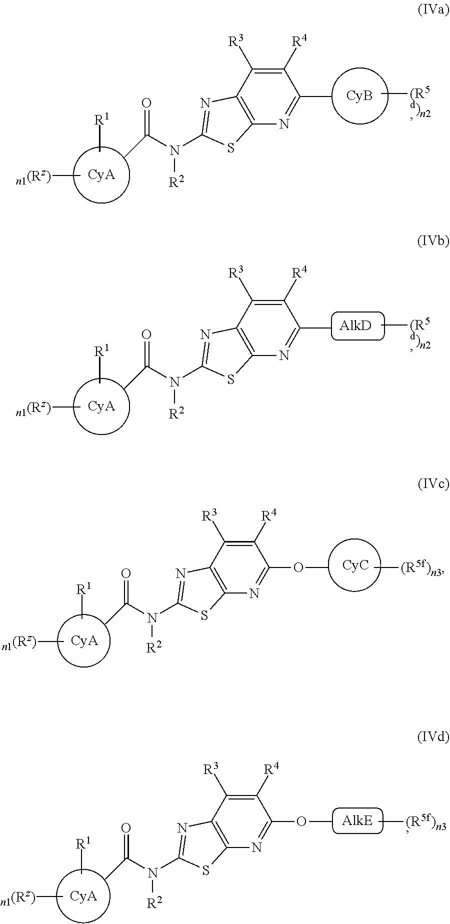

wherein,
CyB and CyC are each independently cycloalkyl, heterocyclyl, aryl or heteroaryl;
AlkD and AlkE are each —C₁₋₈alkyl, —C₂₋₈alkenyl or —C₂₋₈alkynyl;
n2 and n3 are each independently 0, 1, 2, 3, 4, 5, 6, 7 or 8;
R¹, R², R³, R⁴, R^{5d}, R^{5f}, R^{z}, n1 and CyA are each defined as any one of the preceding claims.

Aspect 5. The compound of any one of aspects 2-4, wherein the compound is selected from formula (Va), (Vb), (Vc), (Vd):

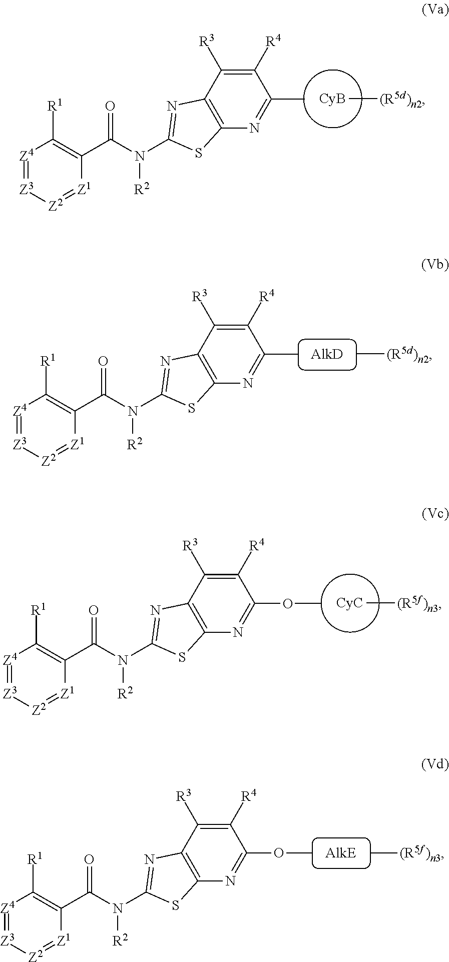

wherein CyB, CyC, AlkD, AlkE, R¹, R², R³, R⁴, R^{5d}, R^{5f}, Z¹, Z², Z³, Z⁴, n2 and n3 are defined as any one of the preceding claims.

Aspect 6. The compound of any one of the preceding aspects, wherein ring CyA is a 5-, 6-, 7-, 8-, 9-, 10-, 11- or 12-membered heteroaromatic ring comprising 1 to 4 heteroatoms selected from N, O, or S as ring member(s).

Aspect 7. The compound of any one of the preceding aspects, wherein ring CyA is a 5- or 6-membered heteroaromatic ring comprising 1 to 2 heteroatoms selected from N, O, or S as ring member(s), preferably is pyridinyl, pyridazinyl, pyrimidinyl, pyrazinyl, imidazolyl, isoimidazolyl, oxazolyl, thiazolyl, furanyl or thiophenyl.

Aspect 8. The compound of any one of the preceding aspects, wherein R¹ is methyl, ethyl, propyl, butyl, pentyl, hexyl, heptyl, octyl, cyclopropyl, cyclobutyl, cyclopentyl, cyclohexyl, cycloheptyl, cyclooctyl, 5- to 8-membered heterocyclyl, phenyl, 5- to 10-membered heteroaryl, oxo, —CN, —OR^{1a}, —SO₂R^{1a}, —SO₂NR^{1a}R^{1b}, —COR^{1a}, —CO₂R^{1a}, —CONR^{1a}R^{1b}, —NR^{1a}R^{1b}, —NR^{1a}COR^{1b}, —NR^{1a}CO₂R^{1b}, —NR^{1a}CONR^{1b}R^{1c}, or —NR^{1a}SO₂R^{1b}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl or heteroaryl is optionally substituted with at least one substituent R^{1d};
R^{1a}, R^{1b} and R^{1c} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl or heteroaryl is optionally substituted with at least one substituent R^{1f}; or
(R^{1a} and R^{1b}), (R^{1b} and R^{1c}) or (R^{1a} and R^{1c}), together with the atom(s) to which they are attached, form a 3-, 4-, 5-, 6-, 7- or 8-membered ring, said ring comprising 0, 1 or 2 heteroatom(s) independently selected from nitrogen, oxygen, or optionally oxidized sulfur as ring member(s), said ring is optionally substituted with at least one substituent R^{1f};
R^{1d} and R^{1f} are each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, aryl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl, heteroaryl, oxo, —CN, —OR^{1g}, —SO₂R^{1g}, —SO₂NR^{1g}R^{1h}, —COR^{1g}, —CO₂R^{1g}, —CONR^{1g}R^{1h}, —NO₂, —NR^{1g}R^{1h}, —NR^{1g}COR^{1h}, —NR^{1g}CO₂R^{1h}, —NR^{1g}CONR^{1h}R¹ⁱ, or —NR^{1g}SO₂R^{1h}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl or heteroaryl is optionally substituted with at least one substituent RJ; or
two R^{1d} or two R^{1f}, together with the atom(s) to which they are attached, form a 3-, 4-, 5-, 6-, 7- or 8-membered ring, said ring comprising 0, 1 or 2 heteroatom(s) independently selected from nitrogen, oxygen, or optionally oxidized sulfur as ring member(s), said ring is optionally substituted with at least one substituent R^{1k};
R^{1g}, R^{1h} and R¹ⁱ are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl or heteroaryl is optionally substituted with at least one substituent —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, aryl, haloaryl, heteroaryl or haloheteroaryl;
R^{1j} and R^{1k} are each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl, heteroaryl, oxo, —CN, —OR^{1l}, —SO₂R^{1l}, —SO₂NR^{1l}R^{1m}, —COR^{1l}, —CO₂R^{1l}, —CONR^{1l}R^{1m}, —NO₂, —NR^{1l}R^{1m}, —NR^{1l}COR^{1m}, —NR^{1l}CO₂R^{1m}, —NR^{1l}CONR^{1m}R¹ⁿ, or —NR^{1l}SO₂R^{1m}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl or heteroaryl is optionally substituted with at least one substituent —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, aryl, haloaryl, heteroaryl or haloheteroaryl;
R^{1l}, R^{1m} and R¹ⁿ are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl or heteroaryl is optionally substituted with at least one substituent —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, aryl, haloaryl, heteroaryl or haloheteroaryl.

Aspect 9. The compound of any one of the preceding aspects, wherein R¹ is cyclopropyl, cyclobutyl, cyclopentyl, cyclohexyl, cycloheptyl, cyclooctyl, morpholinyl, piperidinyl, piperazinyl, dihydropyridinyl, dihydropiperazinyl, phenyl, indazolyl, benzodioxolyl, benzooxazinyl, dihydrobenzooxazinyl, indolyl, benzoimidazolyl, benzooxazolyl, benzofuranyl, quinolinyl or isoquinolinyl; wherein each of said cyclopropyl, cyclobutyl, cyclopentyl, cyclohexyl, cycloheptyl, cyclooctyl, morpholinyl, piperidinyl, piperazinyl, dihydropyridinyl, dihydropiperazinyl, phenyl, indazolyl, benzodioxolyl, benzooxazinyl, dihydrobenzooxazinyl, indolyl, benzoimidazolyl, benzooxazolyl, benzofuranyl, quinolinyl or isoquinolinyl is optionally substituted with at least one substituent R^{1d}.

R^{1d} is each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 6-membered heterocyclyl, aryl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl, heteroaryl, oxo, —CN, —OR^{1g}, —SO₂R^{1g}, —SO₂NR^{1g}R^{1h}, —COR^{1g}, —CO₂R^{1g}, —CONR^{1g}R^{1h}, —NO₂, —NR^{1g}R^{1h}, —NR^{1g}COR^{1h}, —NR^{1g}CO₂R^{1h}, —NR^{1g}CONR^{1h}R¹ⁱ, or —NR^{1g}SO₂R^{1h}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl or heteroaryl is optionally substituted with at least one substituent R^{1j}; or
two R^{1d} together with the atom(s) to which they are attached, form a 3-, 4-, 5-, 6-, 7- or 8-membered ring, said ring comprising 0, 1 or 2 heteroatom(s) independently selected from nitrogen, oxygen, or optionally oxidized sulfur as ring member(s), said ring is optionally substituted with at least one substituent R^{1k};
R^{1g}, R^{1h} and R¹ⁱ are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl or heteroaryl is optionally substituted with at least one substituent —F, —Cl, —Br, —I, hydroxy, methyl, ethyl, propyl, butyl, pentyl, hexyl, heptyl, octyl, -haloC₁₋₈ alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, aryl, haloaryl, heteroaryl or haloheteroaryl;
R^{1j} and R^{1k} are each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl, heteroaryl, oxo, —CN, —OR^{1l}, —SO₂R^{1l}, —SO₂NR^{1l}R^{1m}, —COR^{1l}, —CO₂R^{1l}, —CONR^{1l}R^{1m}, —NO₂, —NR^{1l}R^{1m}, —NR^{1l}COR^{1m}, —NR^{1l}CO₂R^{1m}, —NR^{1l}CONR^{1m}R¹ⁿ, or —NR^{1l}SO₂R^{1m}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl or heteroaryl is optionally substituted with at least one substituent —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, aryl, haloaryl, heteroaryl or haloheteroaryl;
R^{1l}, R^{1m} and R¹ⁿ are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl or heteroaryl is optionally substituted with at least one substituent —F, —Cl, —Br, —I, hydroxy, methyl, ethyl, propyl, butyl, pentyl, hexyl, heptyl, octyl, -haloC₁₋₈ alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, aryl, haloaryl, heteroaryl or haloheteroaryl.

Aspect 10. The compound of any one of the preceding aspects, wherein R¹ is cyclopropyl, cyclobutyl, cyclopentyl, cyclohexyl, cycloheptyl, cyclooctyl, morpholinyl, piperidinyl, piperazinyl, dihydropyridinyl, dihydropiperazinyl, phenyl, indazolyl, benzodioxolyl, benzooxazinyl, dihydrobenzooxazinyl, indolyl, benzoimidazolyl, benzooxazolyl, benzofuranyl, quinolinyl or isoquinolinyl; wherein each of said cyclopropyl, cyclobutyl, cyclopentyl, cyclohexyl, cycloheptyl, cyclooctyl, morpholinyl, piperidinyl, piperazinyl, dihydropyridinyl, dihydropiperazinyl, phenyl, indazolyl, benzodioxolyl, benzooxazinyl, dihydrobenzooxazinyl, indolyl, benzoimidazolyl, benzooxazolyl, benzofuranyl, quinolinyl or isoquinolinyl is optionally substituted with at least one substituent R^{1d}.

R^{1d} is each independently selected from hydrogen, —F, —Cl, —Br, —I, methyl, ethyl, propyl, butyl, pentyl, hexyl, heptyl, octyl, oxo, —CN, cyclopropyl, cyclobutyl, cyclopentyl, cyclohexyl, cycloheptyl, cyclooctyl, heterocyclyl, methoxy, ethoxy, propoxy, butoxy, pentoxy, hexoxy, hepthoxy, octoxy, pyrrolidinyl, imidazolidinyl, pyrazolidinyl, piperidinyl, pyranyl, morpholinyl, oxiranyl, azetidinyl, azocanyl, thiiranyl, azetidinyl, —CF₃, —CONH₂, —CH₂OCH₃, —SO₂(CH₃)₂, —CH₂OH, —NO₂; or
two R^{1d} together with the atom(s) to which they are attached, form a 3-, 4-, 5-, 6-, 7- or 8-membered ring, said ring comprising 0, 1 or 2 heteroatom(s) independently selected from nitrogen, oxygen, or optionally oxidized sulfur as ring member(s), said ring is optionally substituted with at least one substituent R^{1k};
R^{1k} is each independently hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl, heteroaryl, oxo, —CN, methoxy, ethoxy, propoxy, butoxy, pentoxy, hexoxy, hepthoxy, octoxy, —CF₃, —CONH₂, —CH₂OCH₃, —SO₂(CH₃)₂, —CH₂OH, —NO₂; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl or heteroaryl is optionally substituted with at least one substituent —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, aryl, haloaryl, heteroaryl or haloheteroaryl.

In some embodiments, R¹ is phenyl, which is optionally substituted with at least one substituent R^{1d} as disclosed herein. In some further embodiments, R¹ is phenyl, which is substituted with one or two substituents R^{1d} as disclosed herein. In some embodiments, R^{1d} is each independently selected from hydrogen, —F, —Cl, —Br, —I, methyl, ethyl, propyl, butyl, pentyl, hexyl, heptyl, octyl, oxo, —CN, cyclopropyl, cyclobutyl, cyclopentyl, cyclohexyl, cycloheptyl, cyclooctyl, heterocyclyl, methoxy, ethoxy, propoxy, butoxy, pentoxy, hexoxy, hepthoxy, octoxy, pyrrolidinyl, imidazolidinyl, pyrazolidinyl, piperidinyl, pyranyl, morpholinyl, oxiranyl, azetidinyl, azocanyl, thiiranyl, azetidinyl, —CF₃, —CONH₂, —CH₂OCH₃, —SO₂(CH₃)₂, —CH₂OH, —NO₂. In some embodimetns, two R^{1d} together with the atom(s) to which they are attached, form a 3-, 4-, 5-, 6-, 7- or 8-membered ring, said ring comprising 0, 1 or 2 heteroatom(s) independently selected from nitrogen, oxygen, or optionally oxidized sulfur as ring member(s), said ring is optionally substituted with at least one substituent R^{1k}; wherein R^{1k} is each independently hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl, heteroaryl, oxo, —CN, methoxy, ethoxy, propoxy, butoxy, pentoxy, hexoxy, hepthoxy, octoxy, —CF₃, —CONH₂, —CH₂OCH₃, —SO₂(CH₃)₂, —CH₂OH, —NO₂; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl or heteroaryl is optionally substituted with at least one substituent —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, aryl, haloaryl, heteroaryl or haloheteroaryl.

Aspect 11. The compound of any one of the preceding aspects, wherein R¹ is

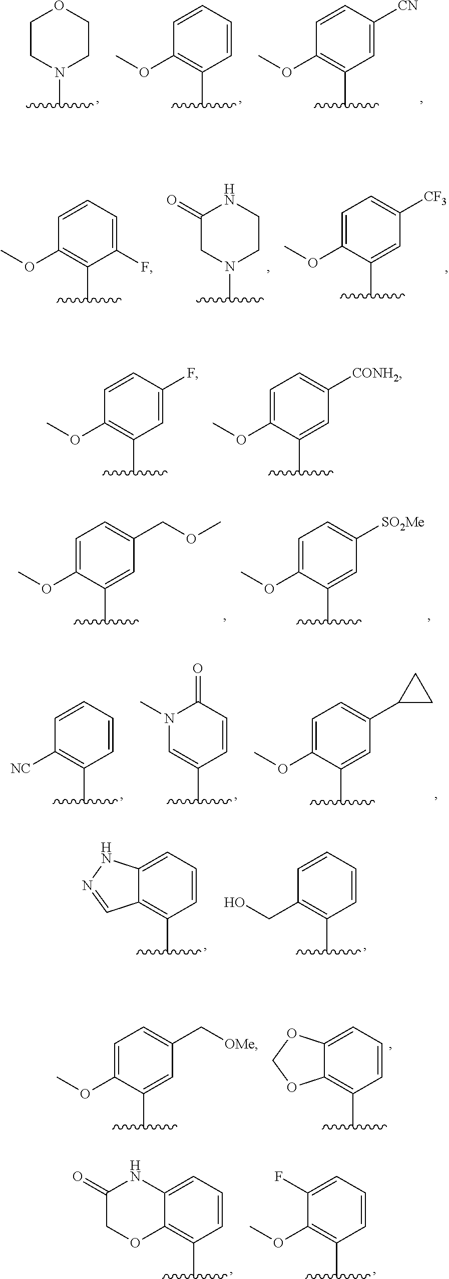

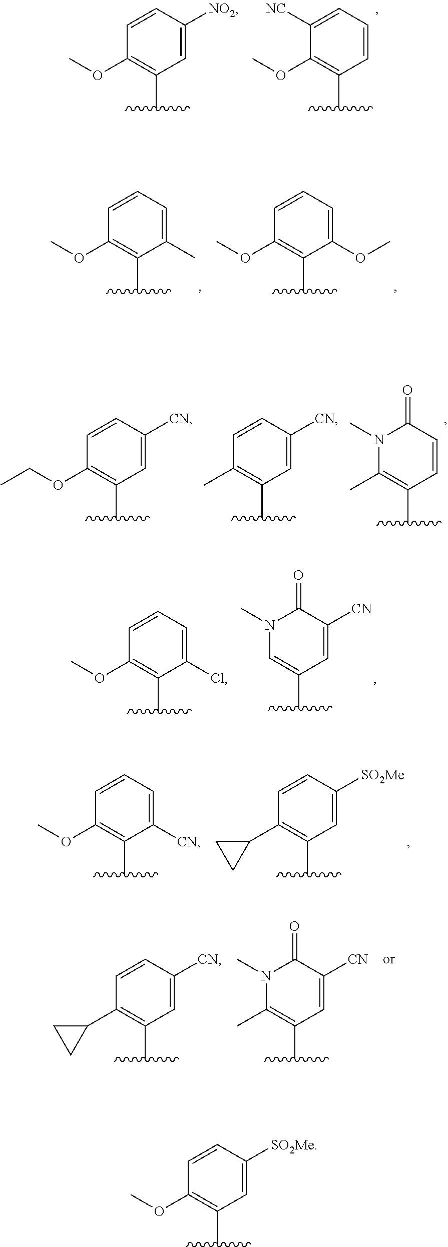

Aspect 12. The compound of any one of the preceding aspects, wherein
R^{z}, at each of its occurrences, is independently hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, oxo, —CN, —OR^{za}, —SO₂R^{za}, —SO₂NR^{za}R^{zb}, —COR^{za}, —CO₂R^{za}, —CONR^{za}R^{zb}, —NR^{za}R^{zb}, —NR^{za}COR^{zb}, —NR^{za}CO₂R^{zb}, —NR^{za}CONR^{zb}R^{zc} or —NR^{za}SO₂R^{zb}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent R^{zd};
R^{za}, R^{zb} and R^{z}C are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent R^{zf}; or
(R^{za} and R^{zb}), (R^{zb} and R^{zc}) or (R^{za} and R^{zc}), together with the atom(s) to which they are attached, form a 3-, 4-, 5-, 6-, 7- or 8-membered ring, said ring comprising 0, 1 or 2 heteroatom(s) independently selected from nitrogen, oxygen, or optionally oxidized sulfur as ring member(s), said ring is optionally substituted with at least one substituent R^{zf};
R^{zd} and R^{zf} are each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, oxo, —CN, —OR^{zg}, —SO₂R^{zg}, —SO₂NR^{zg}R^{zh}, —COR^{zg}, —CO₂R^{zg}, —CONR^{zg}R^{zh}, —NO₂, —NR^{zg}R^{zh}, —NR^{zg}COR^{zh}, —NR^{zg}CO₂R^{zh}, —NR^{zg}CONR^{zh}R^{zi}, or —NR^{zg}SO₂R^{zh}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent R^{z}J; or
two R^{zd} or two R^{zf}, together with the atom(s) to which they are attached, form a 3-, 4-, 5-, 6-, 7- or 8-membered ring, said ring comprising 0, 1 or 2 heteroatom(s) independently selected from nitrogen, oxygen, or optionally oxidized sulfur as ring member(s), said ring is optionally substituted with at least one substituent R^{zk};
R^{zg}, R^{zh} and R^{zi} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent halogen, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, aryl, haloaryl, heteroaryl or haloheteroaryl;
R^{zj} and R^{zk} are each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, hepthoxy, octoxy, cyclopropyl, cyclobutyl, cyclopentyl, cyclohexyl, cycloheptyl, cyclooctyl, heterocyclyl, phenyl, heteroaryl, oxo, —CN; wherein each of said methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, hepthoxy, octoxy, cyclopropyl, cyclobutyl, cyclopentyl, cyclohexyl, cycloheptyl, cyclooctyl, heterocyclyl, phenyl or heteroaryl is optionally substituted with at least one substituent —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl.

Aspect 13. The compound of any one of the preceding aspects, wherein R^{z}, at each of its occurrences, is independently hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, heterocyclyl, phenyl, heteroaryl, oxo, —CN, —OR^{za}, —COR^{za}, —CO₂R^{za}, —CONR^{za}R^{zb} or —NR^{za}R^{zb}; wherein each of said methyl, ethyl, propyl, butyl, pentyl, hexyl, heptyl, octyl, cyclopropyl, cyclobutyl, cyclopentyl, cyclohexyl, cycloheptyl, cyclooctyl, heterocyclyl, phenyl or heteroaryl is optionally substituted with at least one substituent R^{zd};
R^{za} and R^{zb} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl;
R^{zd} is independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, oxo, —CN or —OH.

Aspect 14. The compound of any one of the preceding aspects, wherein R^{z}, at each of its occurrences, is independently H, —CN, —CH₃, —Cl, —CH₂CN, —CF₃, —F, —COOH or —CH₂OH.

Aspect 15. The compound of any one of the preceding aspects, wherein the

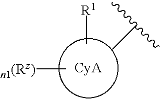

moiety is

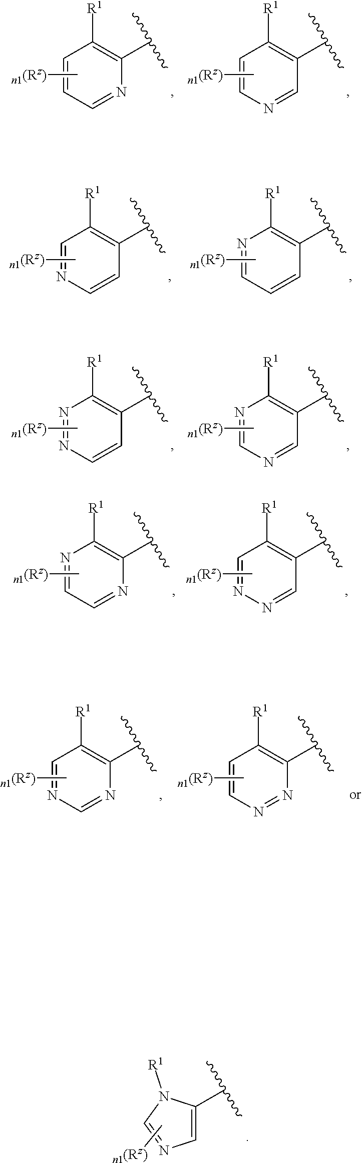

Aspect 16. The compound of any one of the preceding aspects, wherein the

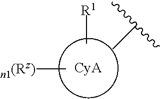

moiety is

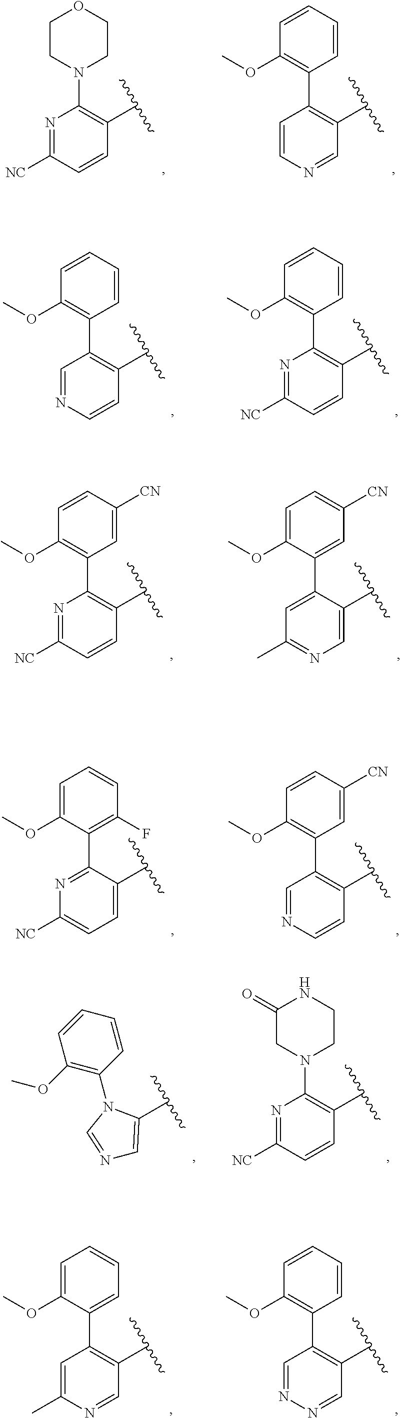

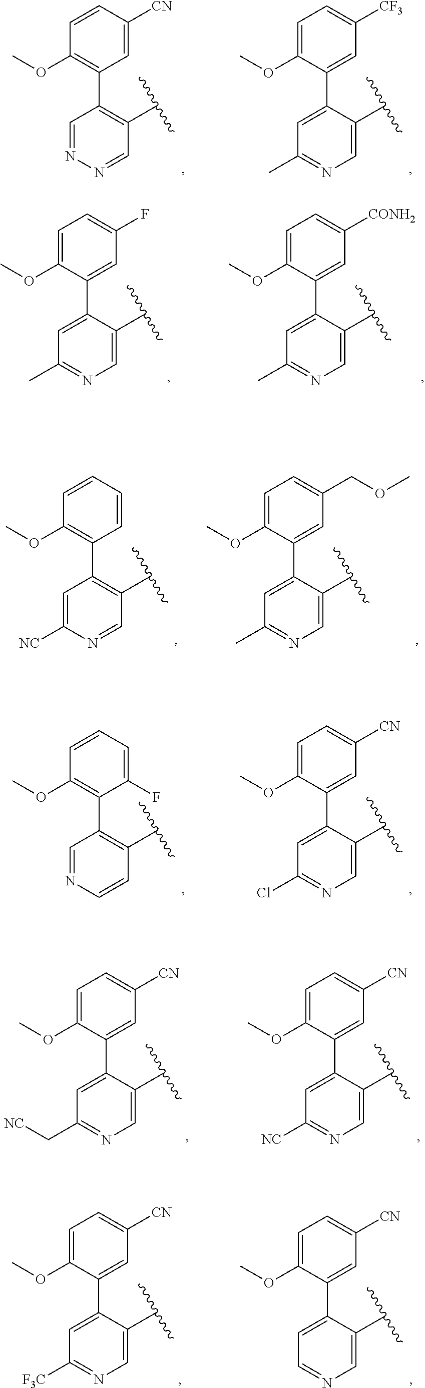

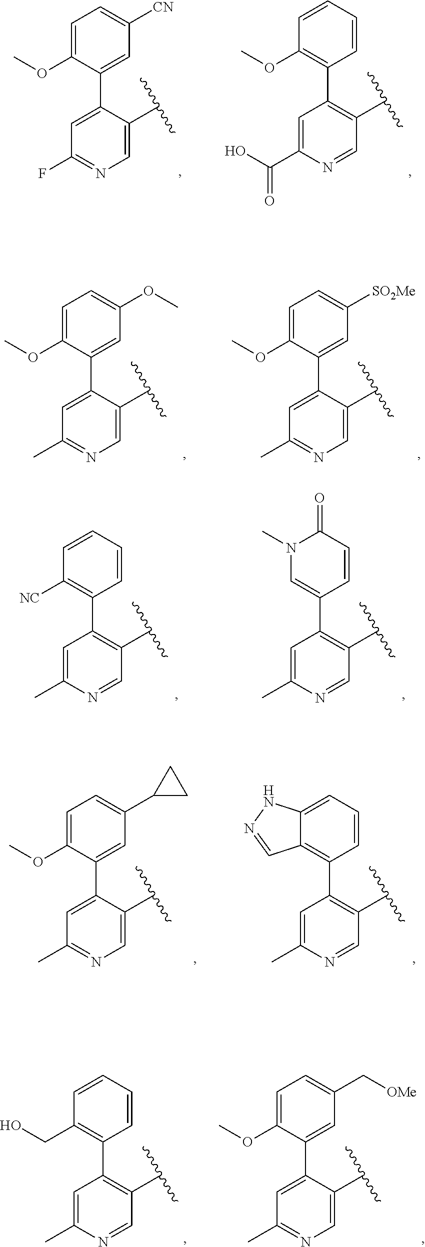

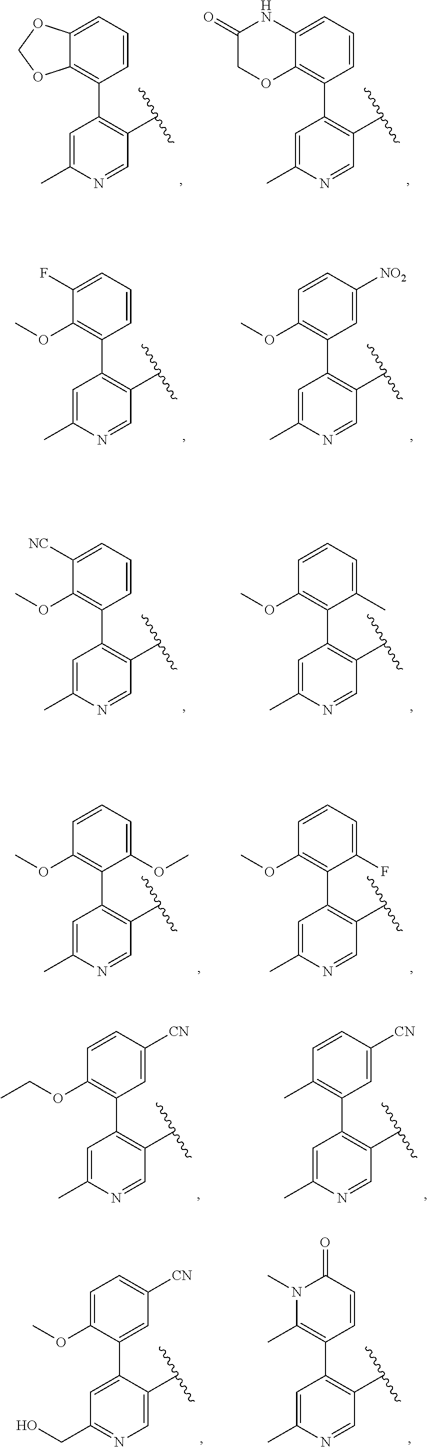

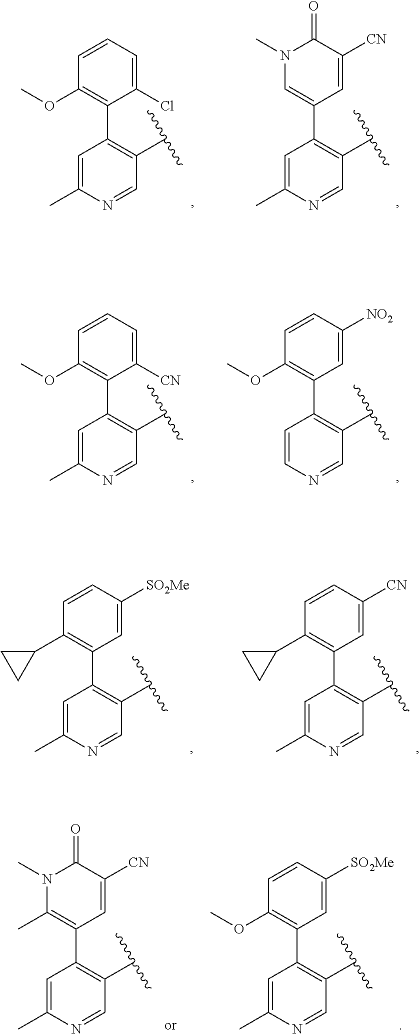

Aspect 17. The compound of any one of the preceding aspects, wherein R² is hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl or 5- to 10-membered heteroaryl; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl or 5- to 10-membered heteroaryl is optionally substituted with at least one substituent R^{2a};
R^{2a} is each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl, 5- to 10-membered heteroaryl, oxo, —CN, —OR^{2b}, —SO₂R^{2b}, —SO₂NR^{2b}R^{2c}, —COR^{2b}, —CO₂R^{2b}, —CONR^{2b}R^{2c}, —NO₂, —NR^{2b}R^{2c}, —NR^{2b}COR^{2c}, —NR^{2b}CO₂R^{2c}, —NR^{2b}CONR^{2c}R^{2d}, or —NR^{2b}SO₂R^{2c}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl or 5- to 10-membered heteroaryl is optionally substituted with at least one substituent R^{2e}; or
two R^{2a}, together with the atom(s) to which they are attached, form a 3-, 4-, 5-, 6-, 7- or 8-membered ring, said ring comprising 0, 1 or 2 heteroatom(s) independently selected from nitrogen, oxygen, or optionally oxidized sulfur as ring member(s), said ring is optionally substituted with at least one substituent R^{2f};
R^{2b}, R^{2c} and R^{2d} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl or 5- to 10-membered heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl or 5- to 10-membered heteroaryl is optionally substituted with at least one substituent halogen, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, 5- to 8-membered heterocyclyl, haloheterocyclyl, phenyl, 5- to 10-membered heteroaryl or haloheteroaryl;
R^{2e} and R^{2f} are each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, hepthoxy, octoxy, cyclopropyl, cyclobutyl, cyclopentyl, cyclohexyl, cycloheptyl, cyclooctyl, 5- to 8-membered heterocyclyl, phenyl or 5- to 10-membered heteroaryl; wherein each of said methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, hepthoxy, octoxy, cyclopropyl, cyclobutyl, cyclopentyl, cyclohexyl, cycloheptyl, cyclooctyl, 5- to 8-membered heterocyclyl, phenyl or 5- to 10-membered heteroaryl is optionally substituted with at least one substituent —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl.

Aspect 18. The compound of any one of the preceding aspects, wherein R² is hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl or 5- to 10-membered heteroaryl; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl or 5- to 10-membered heteroaryl is optionally substituted with at least one substituent —F, —Cl, —Br, —I, hydroxy, methyl, ethyl, propyl, butyl, pentyl, hexyl, heptyl, octyl, -haloC₁₋₈ alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl;
preferably, R² is hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl or 5- to 10-membered heteroaryl;
more preferably, R² is hydrogen.

Aspect 19. The compound of any one of the preceding aspects, wherein R³ and R⁴ are each independently hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl, 5- to 10-membered heteroaryl, oxo, —CN, —OR^{3a}, —SO₂R^{3a}, —SO₂NR^{3a}R^{3b}, —COR^{3a}, —CO₂R^{3a}, —CONR^{3a}R^{3b}, —NR^{3a}R^{3b}, —NR^{3a}COR^{3b}, —NR^{3a}CO₂R^{3b}, —NR^{3a}CONR^{3b}R^{3c}, or —NR^{3a}SO₂R^{3b}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl or 5- to 10-membered heteroaryl is optionally substituted with at least one substituent R^{3a};
R^{3a}, R^{3b} and R^{3c} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent R³; or
(R^{3a} and R^{3b}), (R^{3b} and R^{3c}) or (R^{3a} and R^{3c}), together with the atom(s) to which they are attached, form a 3-, 4-, 5-, 6-, 7- or 8-membered ring, said ring comprising 0, 1 or 2 heteroatom(s) independently selected from nitrogen, oxygen, or optionally oxidized sulfur as ring member(s), said ring is optionally substituted with at least one substituent R^{3f};
R^{3d} and R^{3f} are each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl, 5- to 10-membered heteroaryl, oxo, —CN, —OR^{3g}, —SO₂R^{3g}, —SO₂NR^{3g}R^{3h}, —COR^{3g}, —CO₂R^{3g}, —CONR^{3g}R^{3h}, —NO₂, —NR^{3g}R^{3h}, —NR^{3g}COR^{3h}, —NR^{3g}CO₂R^{3h}, —NR^{3g}CONR^{3h}R³ⁱ or —NR^{3g}SO₂R^{3h}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl, 5- to 10-membered heteroaryl is optionally substituted with at least one substituent halogen, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl;
R^{3g}, R^{3h} and R³ⁱ are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl, 5- to 10-membered heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl or 5- to 10-membered heteroaryl is optionally substituted with at least one substituent halogen, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl.

Aspect 20. The compound of any one of the preceding aspects, wherein R³ and R⁴ are each independently hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl, 5- to 10-membered heteroaryl, oxo, —CN, methoxy, ethoxy, propoxy, butoxy, pentoxy, hexoxy, hepthoxy, octoxy; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl or 5- to 10-membered heteroaryl is optionally substituted with at least one substituent —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl, 5- to 10-membered heteroaryl, oxo, —CN, —NO₂, methoxy, ethoxy, propoxy, butoxy, pentoxy, hexoxy, hepthoxy, octoxy.

Aspect 21. The compound of any one of the preceding aspects, wherein R³ and R⁴ are each independently hydrogen, —F, —Cl, —Br, —I, methyl, ethyl, propyl, butyl, pentyl, hexyl, heptyl, octyl, —C₂₋₈ alkenyl, —C₂₋₈alkynyl, cyclopropyl, cyclobutyl, cyclopentyl, cyclohexyl, cycloheptyl, cyclooctyl, 5- to 8-membered heterocyclyl, phenyl, 5- to 10-membered heteroaryl, oxo, —CN, methoxy, ethoxy, propoxy, butoxy, pentoxy, hexoxy, hepthoxy, octoxy;
preferably, R³ and R⁴ are each independently hydrogen.

Aspect 22. The compound of any one of the preceding aspects, wherein R⁵ is —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl, 5- to 10-membered heteroaryl, oxo, —CN, —OR^{5a}, —SO₂R^{5a}, —SO₂NR^{5a}R^{5b}, —COR^{5a}, —CO₂R^{5a}, —CONR^{5a}R^{5b}, —NR^{5a}R^{5b}, —NR^{5a}COR^{5b}, —NR^{5a}CO₂R^{5b}, —NR^{5a}CONR^{5b}R^{5c}, or —NR^{5a}SO₂R^{5b}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl or 5- to 10-membered heteroaryl is optionally substituted with at least one substituent R^{5d};
R^{5a}, R^{5b} and R^{5c} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl or 5- to 10-membered heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl, 5- to 10-membered heteroaryl is optionally substituted with at least one substituent R^{5f}; or
(R^{5a} and R^{5b}), (R^{5b} and R^{5c}) or (R^{5a} and R^{5c}), together with the atom(s) to which they are attached, form a 3-, 4-, 5-, 6-, 7- or 8-membered ring, said ring comprising 0, 1 or 2 heteroatom(s) independently selected from nitrogen, oxygen, or optionally oxidized sulfur as ring member(s), said ring is optionally substituted with at least one substituent R^{5f};
R^{5d} and R^{5f} are each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl, 5- to 10-membered heteroaryl, oxo, —CN, —OR^{5g}, —SO₂R^{5g}, —SO₂NR^{5g}R^{5h}, —COR^{5g}, —CO₂R^{5g}, —CONR^{5g}R^{5h}, —NO₂, —NR^{5g}R^{5h}, —NR^{5g}COR^{5h}, —NR^{5g}CO₂R^{5h}, —NR^{5g}CONR^{5h}R⁵ⁱ, or —NR^{5g}SO₂R^{5h}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl or 5- to 10-membered heteroaryl is optionally substituted with at least one substituent R^{5j}; or
two R^{5d} or two R^{5f}, together with the atom(s) to which they are attached, form a 3-, 4-, 5-, 6-, 7- or 8-membered ring, said ring comprising 0, 1 or 2 heteroatom(s) independently selected from nitrogen, oxygen, or optionally oxidized sulfur as ring member(s), said ring is optionally substituted with at least one substituent R^{5k};
R^{5g}, R^{5h} and R⁵ⁱ are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl or 5- to 10-membered heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl or 5- to 10-membered heteroaryl is optionally substituted with at least one substituent halogen, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, 5- to 8-membered heterocyclyl, haloheterocyclyl, phenyl or 5- to 10-membered heteroaryl; or
(R^{5g} and R^{5h}), (R^{5h} and R⁵ⁱ) or (R^{5g} and R⁵ⁱ), together with the atom(s) to which they are attached, form a 3-, 4-, 5-, 6-, 7- or 8-membered ring, said ring comprising 0, 1 or 2 heteroatom(s) independently selected from nitrogen, oxygen, or optionally oxidized sulfur as ring member(s), said ring is optionally substituted with at least one substituent —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, 5- to 8-membered heterocyclyl, haloheterocyclyl, phenyl, haloaryl, heteroaryl or haloheteroary;
R^{5j} and R^{5k} are each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl, heteroaryl, oxo, —CN, —OR^{5l}, —SO₂R^{5l}, —SO₂NR^{5l}R^{5m}, —COR^{5l}, —CO₂R^{5l}, —CONR^{5l}R^{5m}, —NO₂, —NR^{5l}R^{5m}, —NR^{5l}COR^{5m}, —NR^{5l}CO₂R^{5m}, —NR^{5l}CONR^{5m}R⁵ⁿ, or —NR^{5l}SO₂R^{5m}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl or heteroaryl is optionally substituted with at least one substituent —F, —Cl, —Br, —I, hydroxy, methyl, ethyl, propyl, butyl, pentyl, hexyl, heptyl, octyl, -haloC₁₋₈ alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, 5- to 8-membered heterocyclyl, haloheterocyclyl, phenyl, haloaryl, heteroaryl or haloheteroaryl;
R^{5l}, R^{5m} and R⁵ⁿ are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl, or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl, or heteroaryl is optionally substituted with at least one substituent halogen, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, 5- to 8-membered heterocyclyl, haloheterocyclyl, phenyl, haloaryl, heteroaryl or haloheteroaryl.

Aspect 23. The compound of any one of the preceding aspects, wherein R⁵ is methyl, ethyl, propyl, butyl, pentyl, hexyl, heptyl, octyl, vinyl, ethynyl, cyclopropyl, cyclobutyl, cyclopentyl, cyclohexyl, cycloheptyl, cyclooctyl, 5- to 8-membered heterocyclyl, phenyl, 5- to 10-membered heteroaryl, —OR^{5a}, —CONR^{5a}R^{5b}, —COR^{5a}, —CO₂R^{5a}, —NR^{5a}R^{5b} or —NR^{5a}COR^{5b}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl or 5- to 10-membered heteroaryl is optionally substituted with at least one substituent R^{5d};
R^{5a}, R^{5b} and R^{5c} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl or 5- to 10-membered heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl, 5- to 10-membered heteroaryl is optionally substituted with at least one substituent R^{5g}; or
(R^{5a} and R^{5b}), (R^{5b} and R^{5c}) or (R^{5a} and R^{5c}), together with the atom(s) to which they are attached, form a 3-, 4-, 5-, 6-, 7- or 8-membered ring, said ring comprising 0, 1 or 2 heteroatom(s) independently selected from nitrogen, oxygen, or optionally oxidized sulfur as ring member(s), said ring is optionally substituted with at least one substituent R^{5f};
R^{5d} and R^{5f} are each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl, 5- to 10-membered heteroaryl, oxo, —CN, —OR^{5g}, —SO₂R^{5g}, —SO₂NR^{5g}R^{5h}, —COR^{5g}, —CO₂R^{5g}, —CONR^{5g}R^{5h}, —NO₂, —NR^{5g}R^{5h}, —NR^{5g}COR^{5h}, —NR^{5g}CO₂R^{5h}, —NR^{5g}CONR^{5h}R⁵ⁱ, or —NR^{5g}SO₂R^{5h}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl or 5- to 10-membered heteroaryl is optionally substituted with at least one substituent R^{5j}; or
two R^{5d} or two R^{5f}, together with the atom(s) to which they are attached, form a 3-, 4-, 5-, 6-, 7- or 8-membered ring, said ring comprising 0, 1 or 2 heteroatom(s) independently selected from nitrogen, oxygen, or optionally oxidized sulfur as ring member(s), said ring is optionally substituted with at least one substituent R^{1k};
R^{5g}, R^{5h} and R⁵ⁱ are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl or 5- to 10-membered heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl or 5- to 10-membered heteroaryl is optionally substituted with at least one substituent halogen, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, 5- to 8-membered heterocyclyl, haloheterocyclyl, phenyl or 5- to 10-membered heteroaryl; or
(R^{5g} and R^{5h}), (R^{5h} and R⁵ⁱ) or (R^{5g} and R⁵ⁱ), together with the atom(s) to which they are attached, form a 3-, 4-, 5-, 6-, 7- or 8-membered ring, said ring comprising 0, 1 or 2 heteroatom(s) independently selected from nitrogen, oxygen, or optionally oxidized sulfur as ring member(s), said ring is optionally substituted with at least one substituent —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, 5- to 8-membered heterocyclyl, haloheterocyclyl, phenyl, haloaryl, heteroaryl or haloheteroary;
R^{5j} and R^{Sk} are each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl, heteroaryl, oxo, —CN, —OR^{5l}, —SO₂R^{5l}, —SO₂NR^{5l}R^{5m}, —COR^{5l}, —CO₂R^{5l}, —CONR^{5l}R^{5m}, —NO₂, —NR^{5l}R^{5m}, —NR^{5l}COR^{5m}, —NR^{5l}CO₂R^{5m}, —NR^{5l}CONR^{5m}R⁵ⁿ, or —NR^{5l}SO₂R^{5m}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl or heteroaryl is optionally substituted with at least one substituent —F, —Cl, —Br, —I, hydroxy, methyl, ethyl, propyl, butyl, pentyl, hexyl, heptyl, octyl, -haloC₁₋₈ alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, 5- to 8-membered heterocyclyl, haloheterocyclyl, phenyl, haloaryl, heteroaryl or haloheteroaryl;
R^{5l}, R^{5m} and R⁵ⁿ are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl, or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl, or heteroaryl is optionally substituted with at least one substituent halogen, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, 5- to 8-membered heterocyclyl, haloheterocyclyl, phenyl, haloaryl, heteroaryl or haloheteroaryl.

Aspect 24. The compound of any one of the preceding aspects, wherein R⁵ is

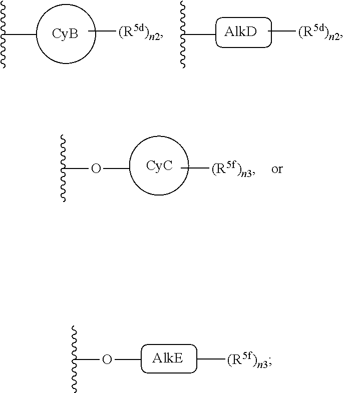

wherein CyB, AlkD, CyC, AlkE, R^{5d}, R^{5f}, n2 and n3 are each defined as any one of the preceding claims.

Aspect 25. The compound of any one of the preceding aspects, wherein R⁵ is

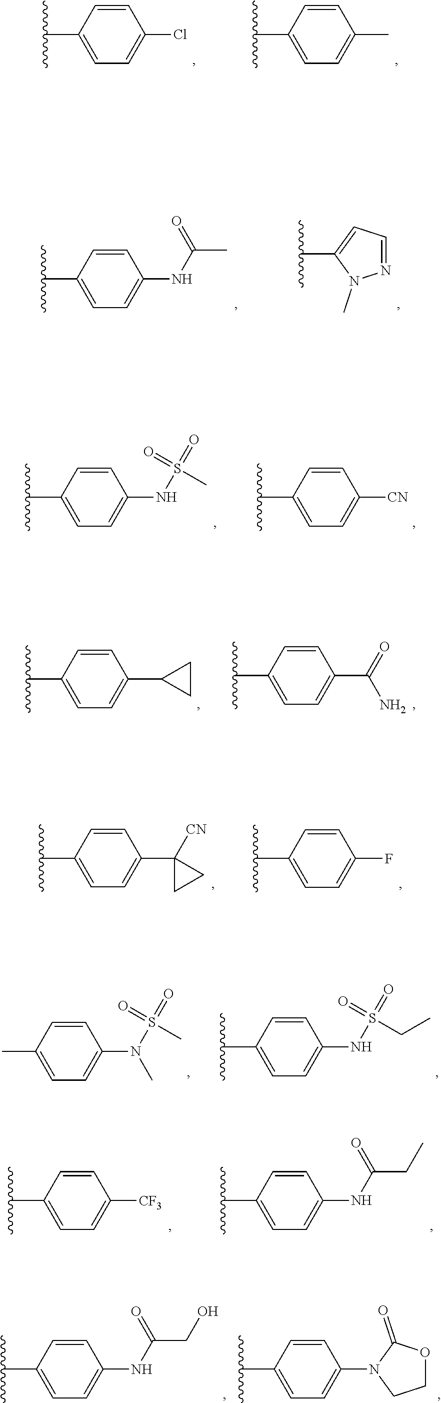

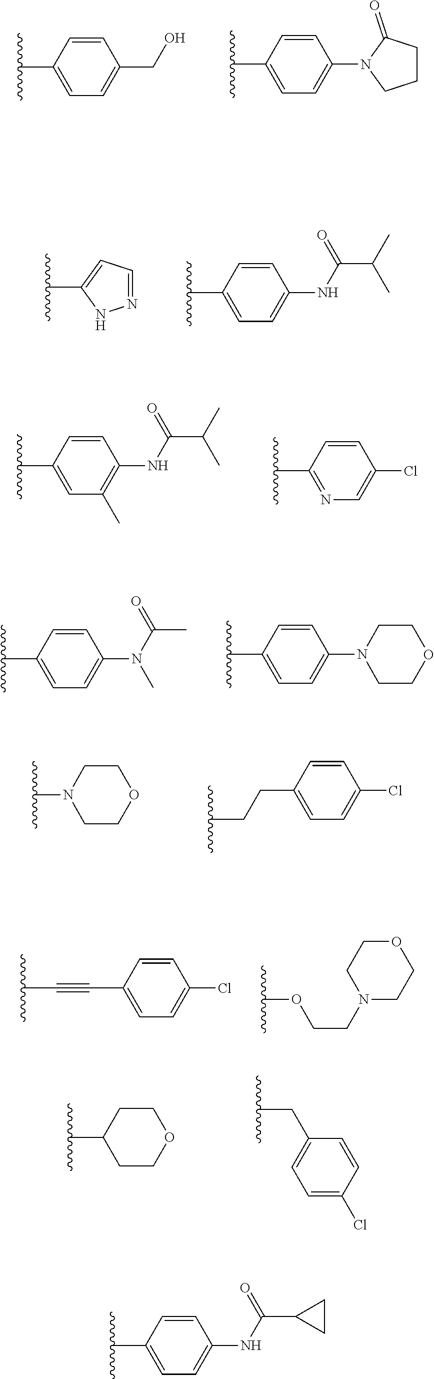

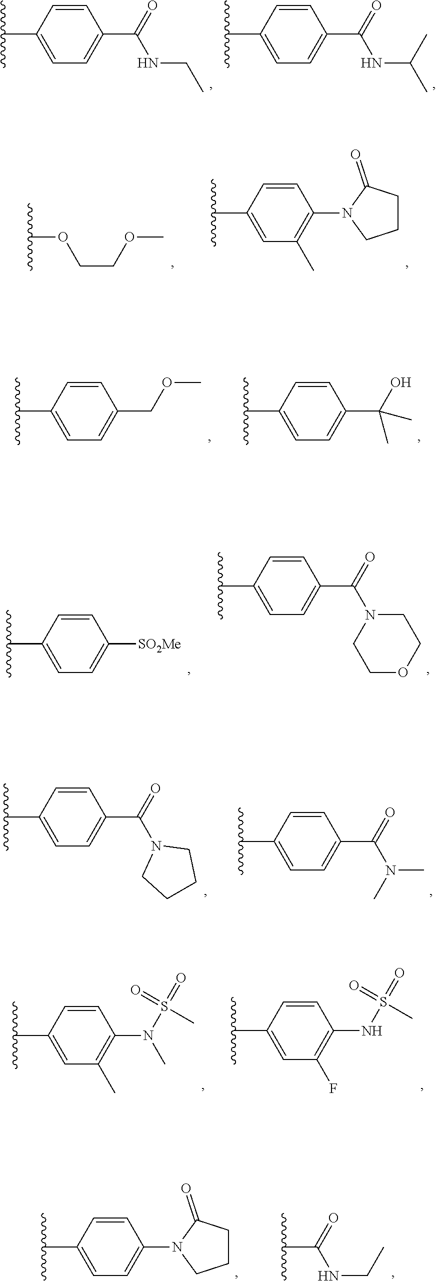

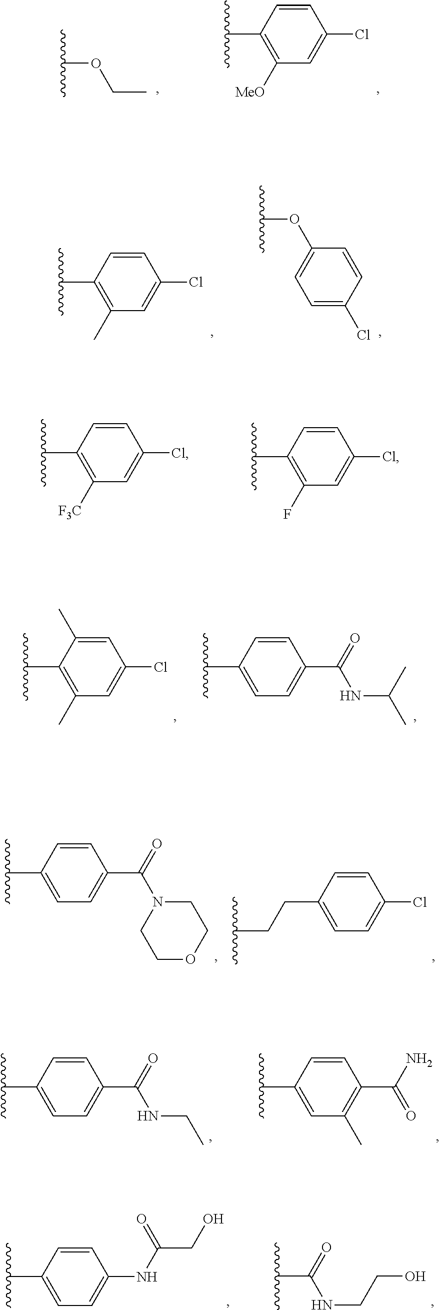

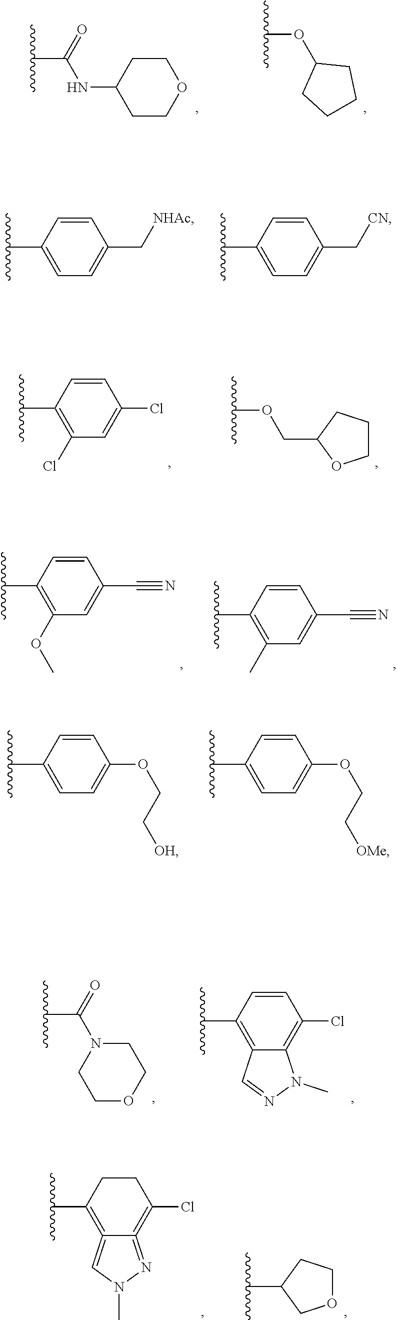

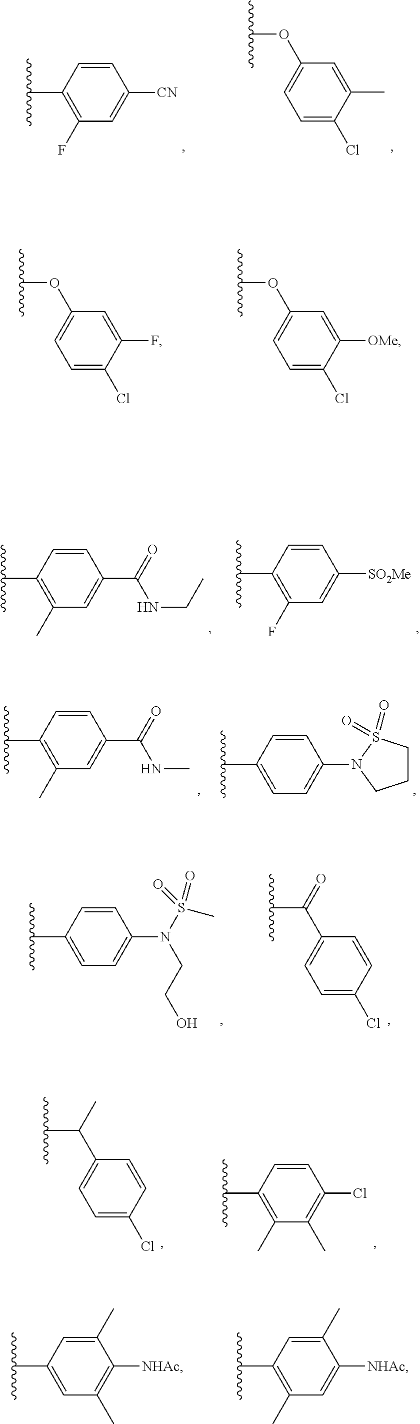

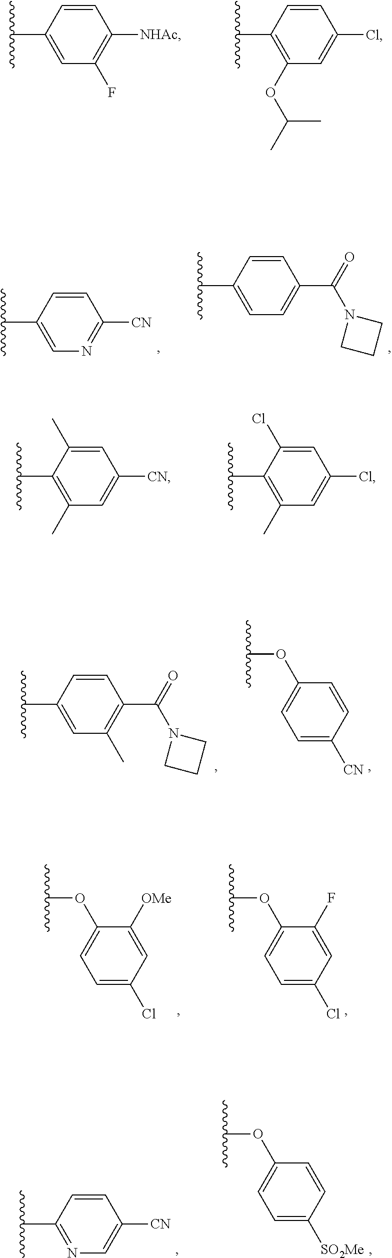

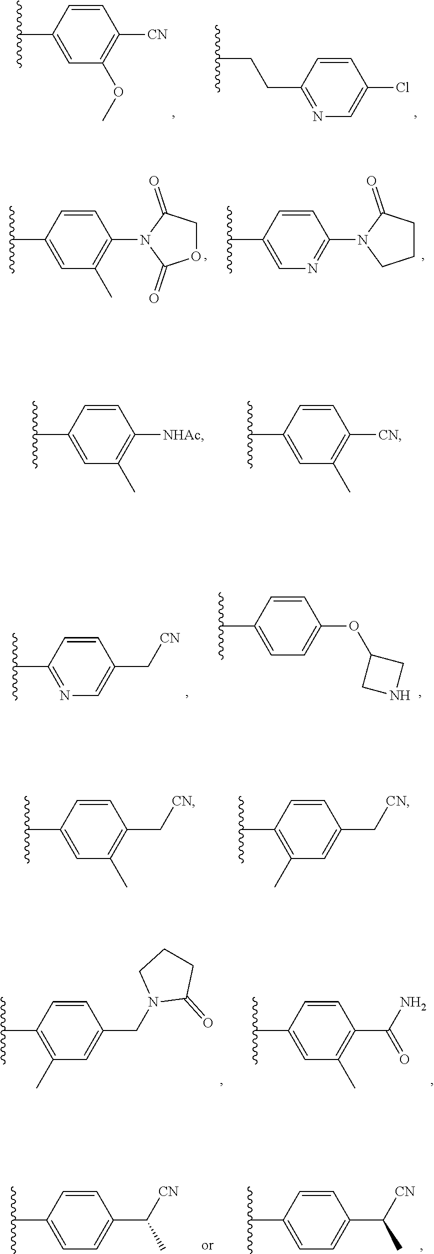

Aspect 26. The compound of any one of the preceding aspects, wherein the compound is selected from

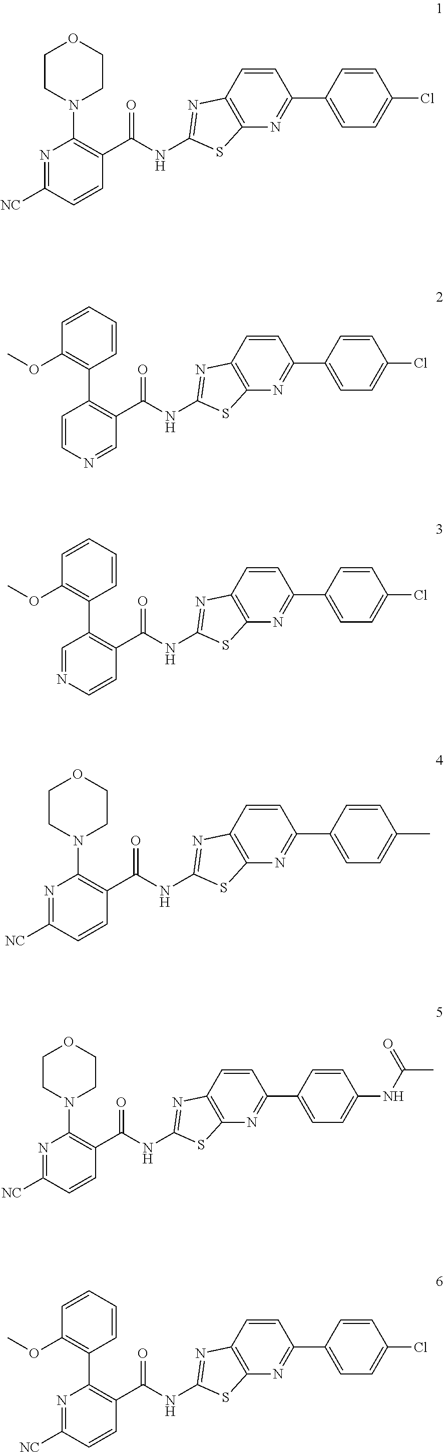

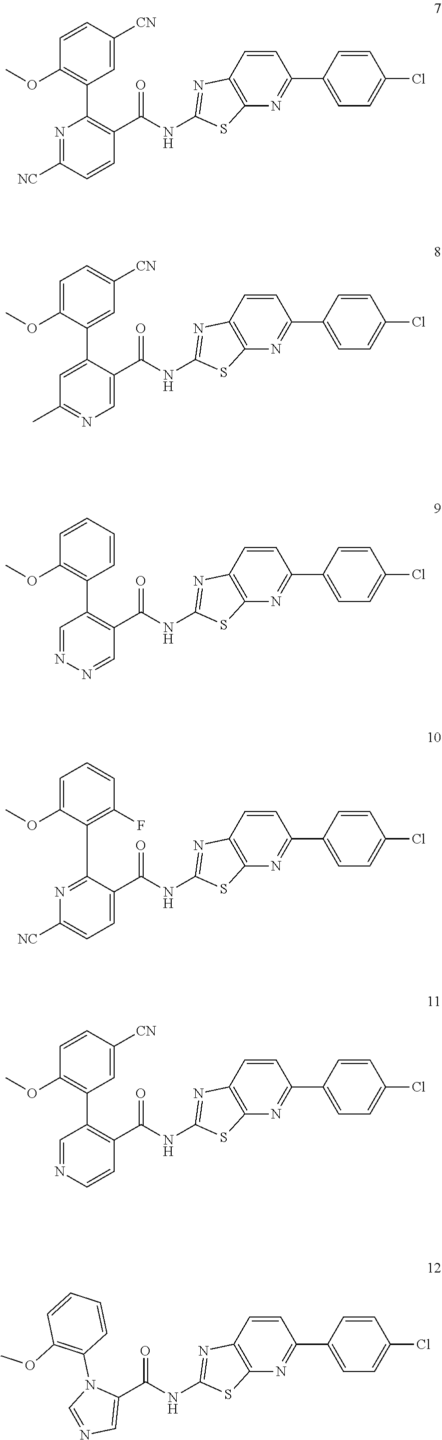

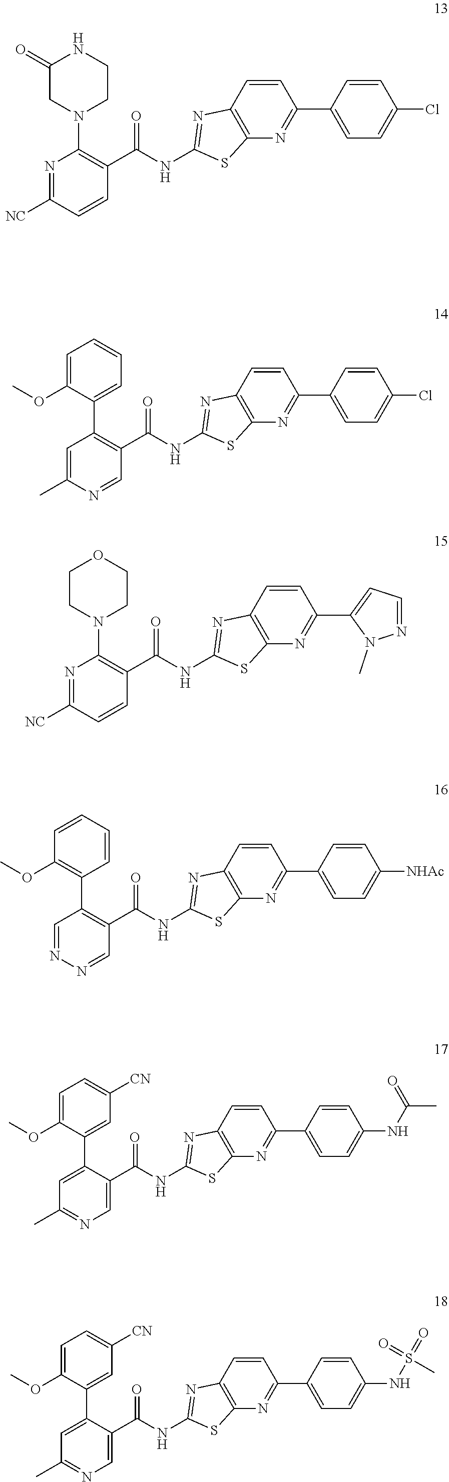

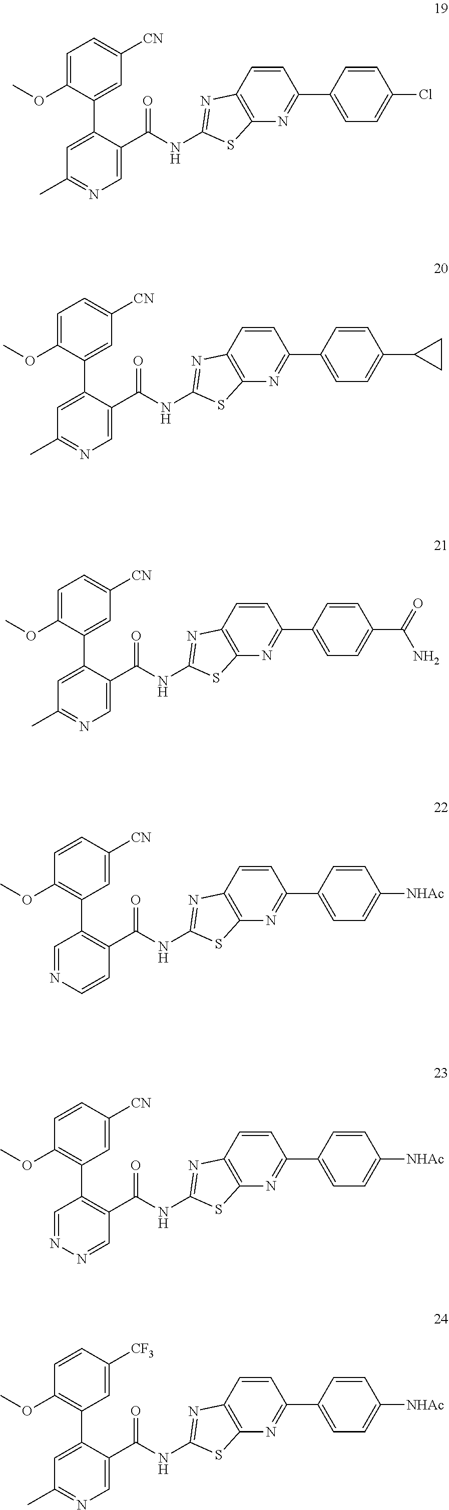 [SMILES: COc1ccc(C#N)cc1-c1cc(C)ncc1C(=O)Nc1nc2ccc(-c3ccc(Cl)cc3)nc2s1 ; COc1ccc(C#N)cc1-c1cc(C)ncc1C(=O)Nc1nc2ccc(-c3ccc(C4CC4)cc3)nc2s1 ; COc1ccc(C#N)cc1-c1cc(C)ncc1C(=O)Nc1nc2ccc(-c3ccc(C(N)=O)cc3)nc2s1 ; COc1ccc(C#N)cc1-c1cnccc1C(=O)Nc1nc2ccc(-c3ccc(NC(C)=O)cc3)nc2s1 ; COc1ccc(C#N)cc1-c1cnncc1C(=O)Nc1nc2ccc(-c3ccc(NC(C)=O)cc3)nc2s1 ; COc1ccc(C(F)(F)F)cc1-c1cc(C)ncc1C(=O)Nc1nc2ccc(-c3ccc(NC(C)=O)cc3)nc2s1]

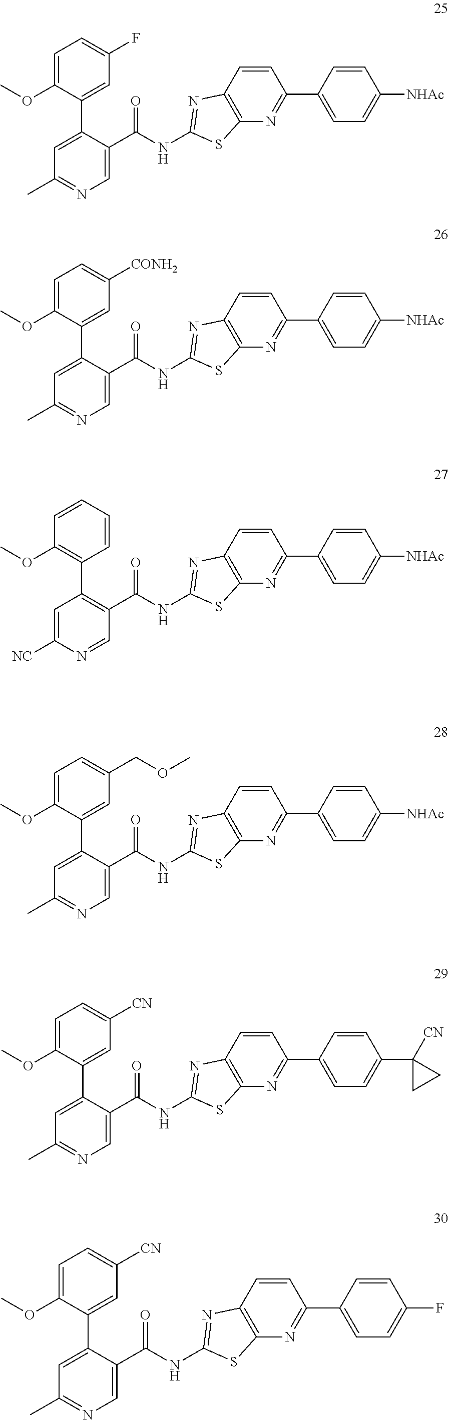 [SMILES: COc1ccc(F)cc1-c1cc(C)ncc1C(=O)Nc1nc2ccc(-c3ccc(NC(C)=O)cc3)nc2s1 ; COc1ccc(C(N)=O)cc1-c1cc(C)ncc1C(=O)Nc1nc2ccc(-c3ccc(NC(C)=O)cc3)nc2s1 ; COc1ccccc1-c1cc(C#N)ncc1C(=O)Nc1nc2ccc(-c3ccc(NC(C)=O)cc3)nc2s1 ; COCc1ccc(OC)c(-c2cc(C)ncc2C(=O)Nc2nc3ccc(-c4ccc(NC(C)=O)cc4)nc3s2)c1 ; COc1ccc(C#N)cc1-c1cc(C)ncc1C(=O)Nc1nc2ccc(-c3ccc(C4(C#N)CC4)cc3)nc2s1 ; COc1ccc(C#N)cc1-c1cc(C)ncc1C(=O)Nc1nc2ccc(-c3ccc(F)cc3)nc2s1]

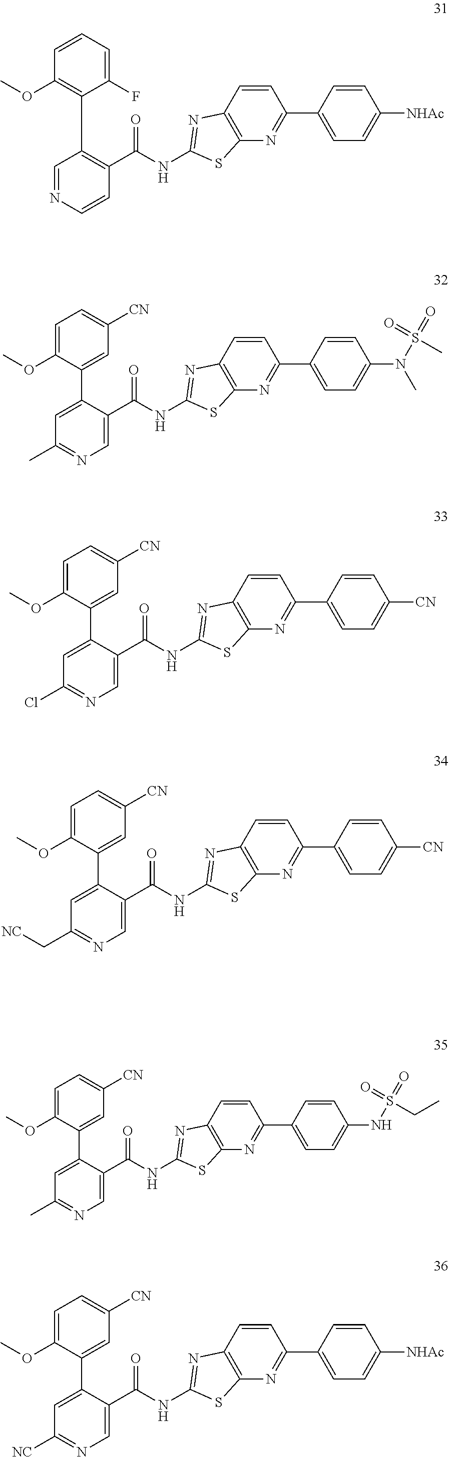

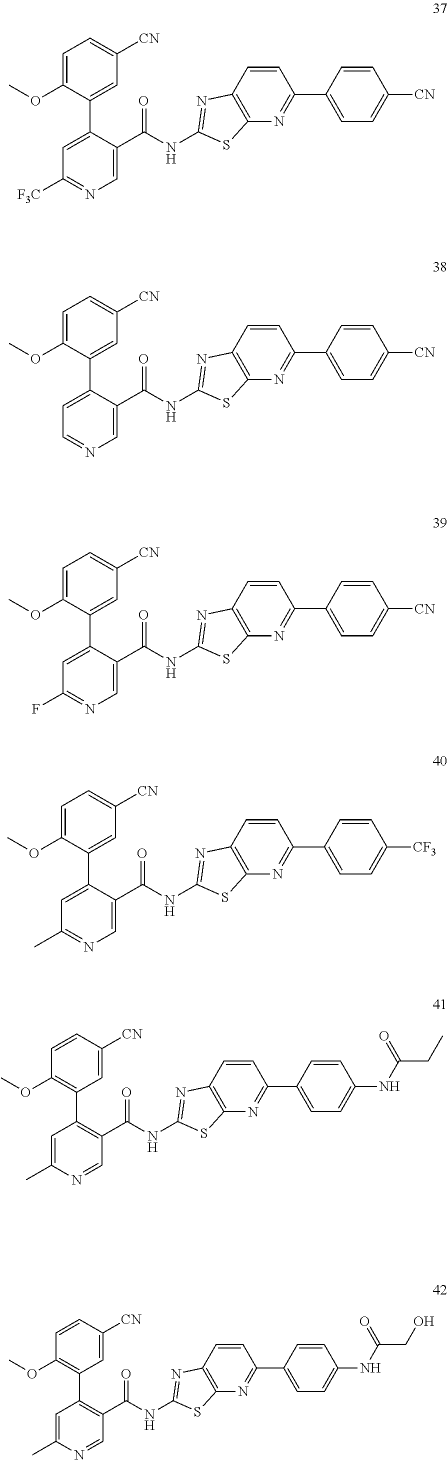

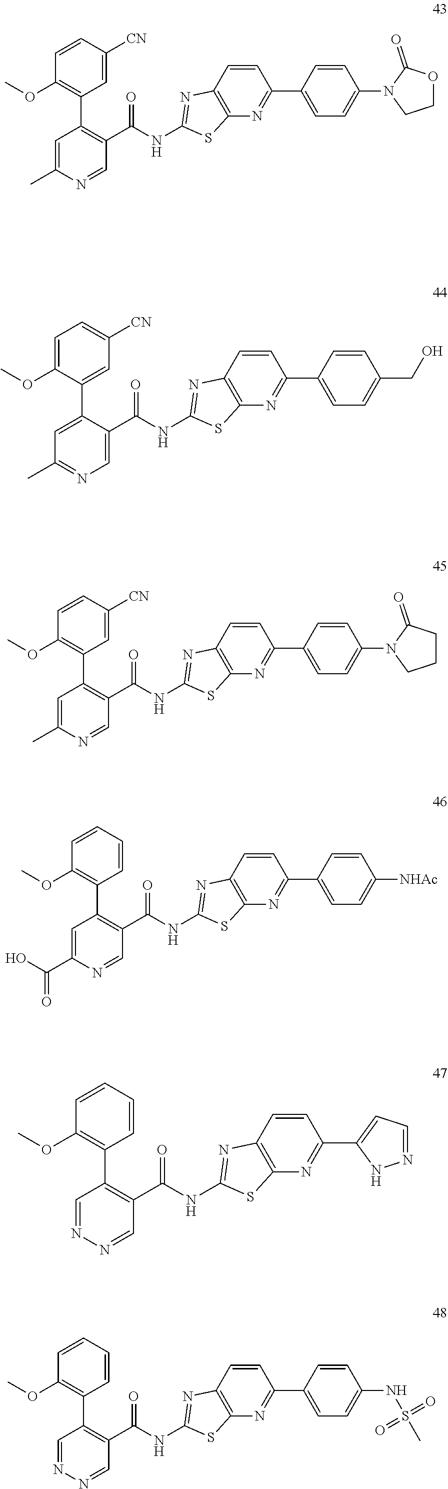 [SMILES: COc1ccc(C#N)cc1-c1cc(C)ncc1C(=O)Nc1nc2ccc(-c3ccc(N4CCOC4=O)cc3)nc2s1 ; COc1ccc(C#N)cc1-c1cc(C)ncc1C(=O)Nc1nc2ccc(-c3ccc(CO)cc3)nc2s1 ; COc1ccc(C#N)cc1-c1cc(C)ncc1C(=O)Nc1nc2ccc(-c3ccc(N4CCCC4=O)cc3)nc2s1 ; COc1ccccc1-c1cc(C(=O)O)ncc1C(=O)Nc1nc2ccc(-c3ccc(NC(C)=O)cc3)nc2s1 ; COc1ccccc1-c1cnncc1C(=O)Nc1nc2ccc(-c3ccn[nH]3)nc2s1 ; COc1ccccc1-c1cnncc1C(=O)Nc1nc2ccc(-c3ccc(NS(C)(=O)=O)cc3)nc2s1]

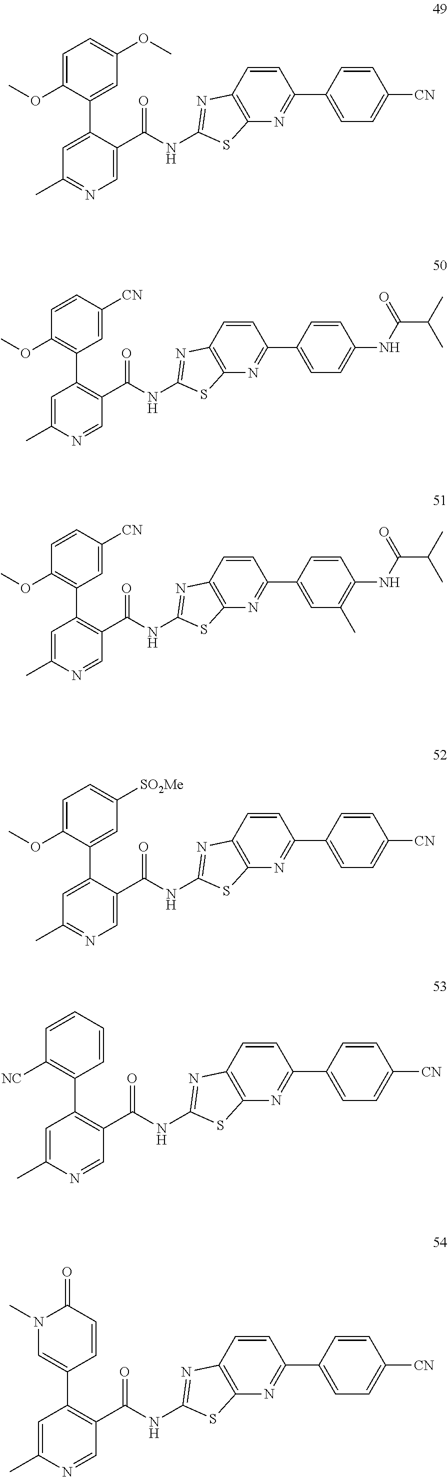

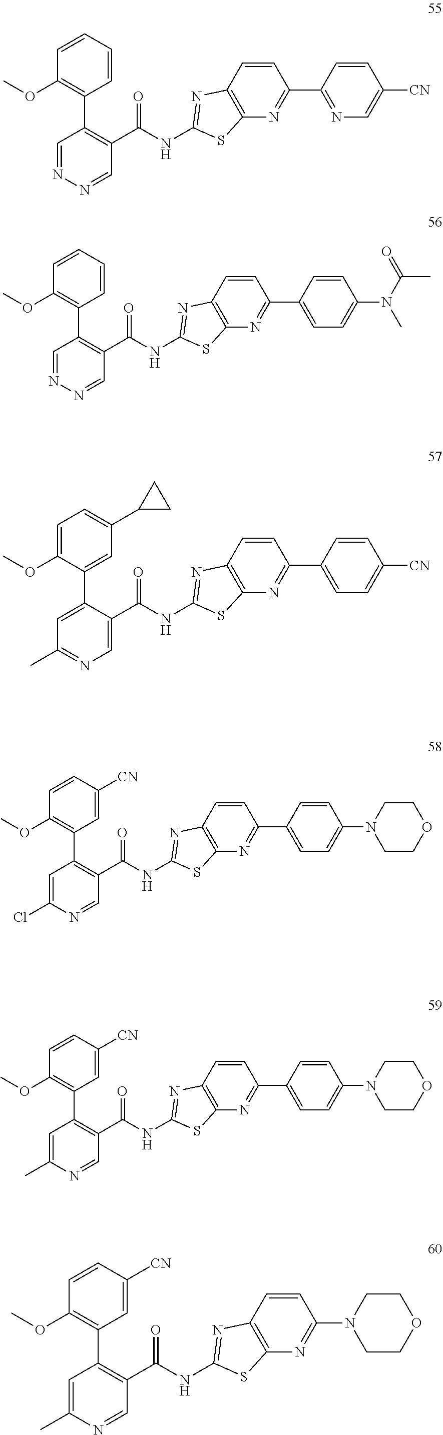

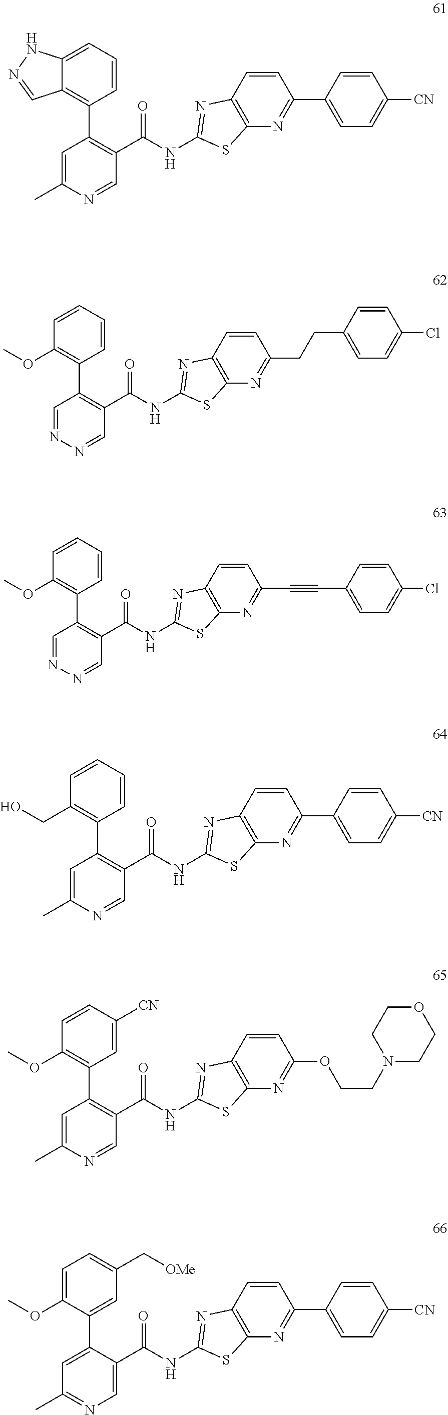 [SMILES: Cc1cc(-c2cccc3[nH]ncc23)c(C(=O)Nc2nc3ccc(-c4ccc(C#N)cc4)nc3s2)cn1 ; COc1ccccc1-c1cnncc1C(=O)Nc1nc2ccc(CCc3ccc(Cl)cc3)nc2s1 ; COc1ccccc1-c1cnncc1C(=O)Nc1nc2ccc(C#Cc3ccc(Cl)cc3)nc2s1 ; Cc1cc(-c2ccccc2CO)c(C(=O)Nc2nc3ccc(-c4ccc(C#N)cc4)nc3s2)cn1 ; COc1ccc(C#N)cc1-c1cc(C)ncc1C(=O)Nc1nc2ccc(OCCN3CCOCC3)nc2s1 ; COCc1ccc(OC)c(-c2cc(C)ncc2C(=O)Nc2nc3ccc(-c4ccc(C#N)cc4)nc3s2)c1]

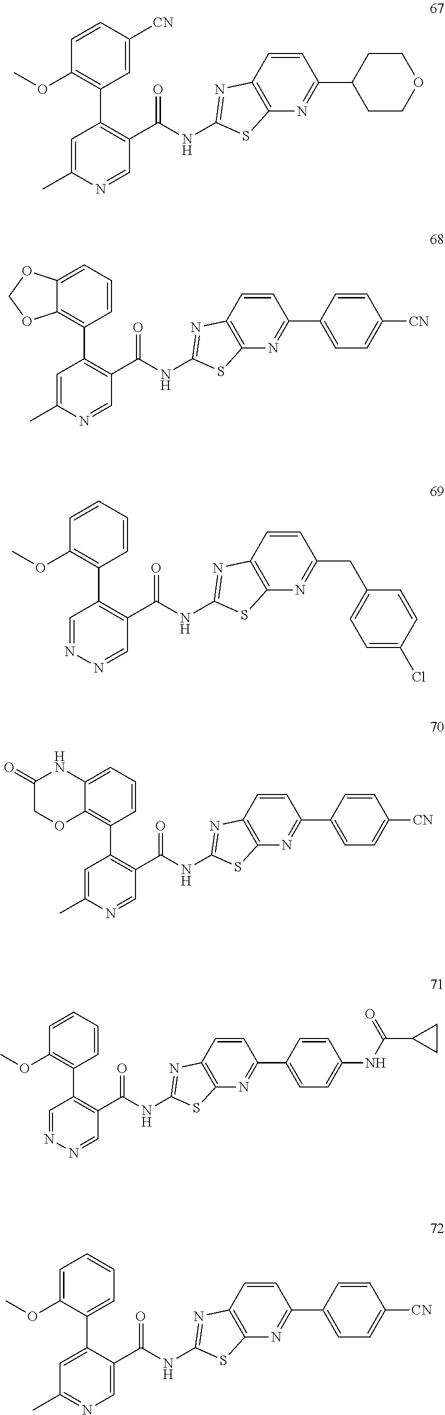 [SMILES: COc1ccc(C#N)cc1-c1cc(C)ncc1C(=O)Nc1nc2ccc(C3CCOCC3)nc2s1 ; Cc1cc(-c2cccc3c2OCO3)c(C(=O)Nc2nc3ccc(-c4ccc(C#N)cc4)nc3s2)cn1 ; COc1ccccc1-c1cnncc1C(=O)Nc1nc2ccc(Cc3ccc(Cl)cc3)nc2s1 ; Cc1cc(-c2cccc3c2OCC(=O)N3)c(C(=O)Nc2nc3ccc(-c4ccc(C#N)cc4)nc3s2)cn1 ; COc1ccccc1-c1cnncc1C(=O)Nc1nc2ccc(-c3ccc(NC(=O)C4CC4)cc3)nc2s1 ; COc1ccccc1-c1cc(C)ncc1C(=O)Nc1nc2ccc(-c3ccc(C#N)cc3)nc2s1]

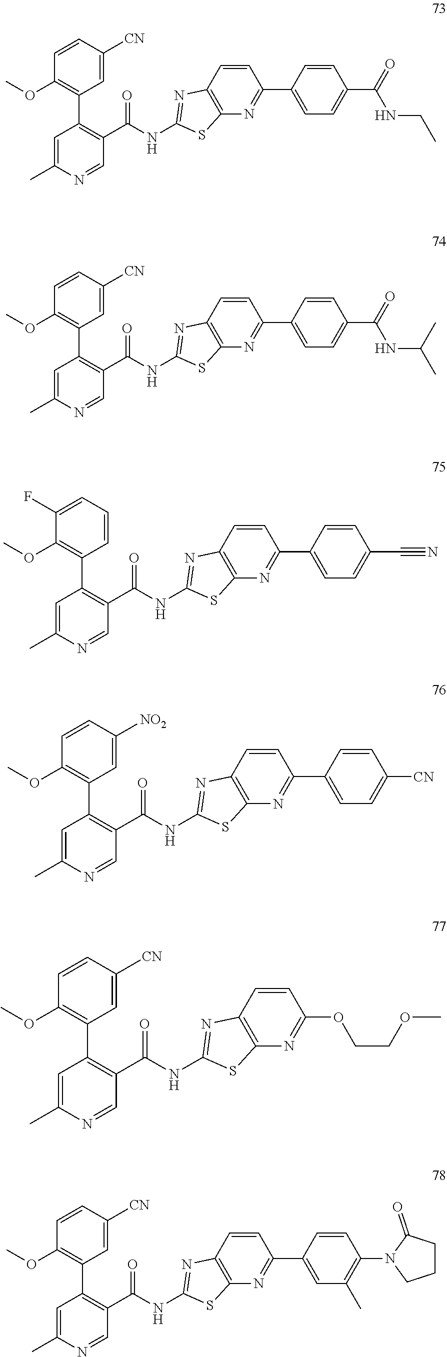

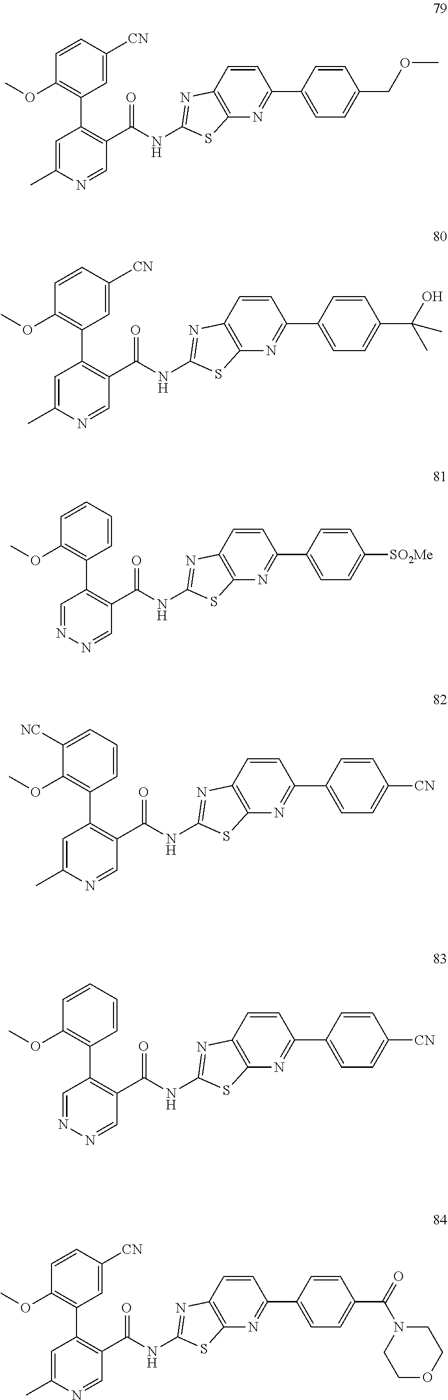

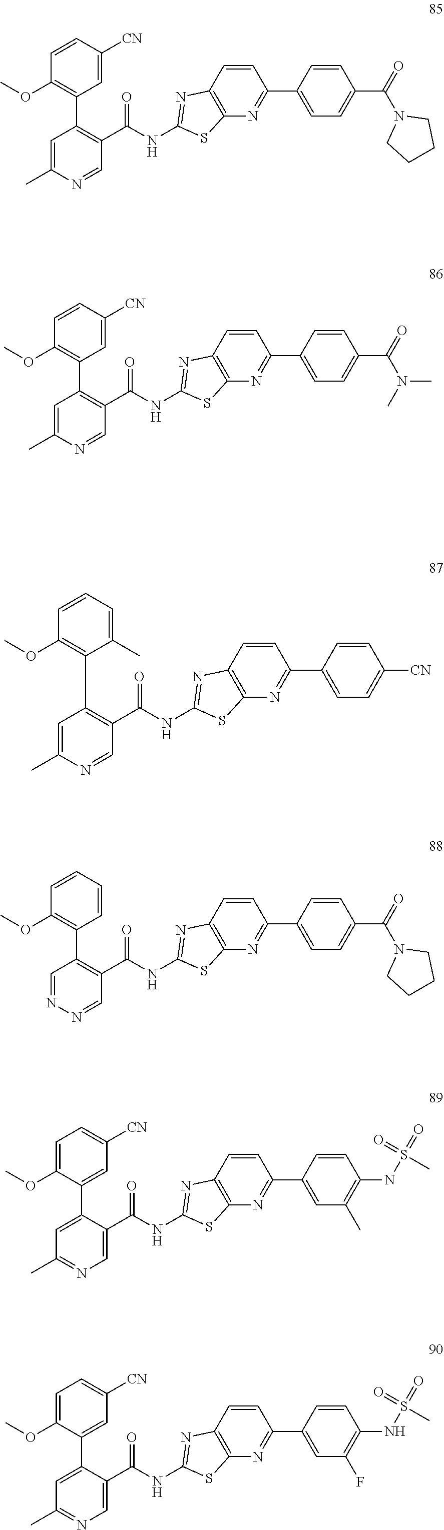

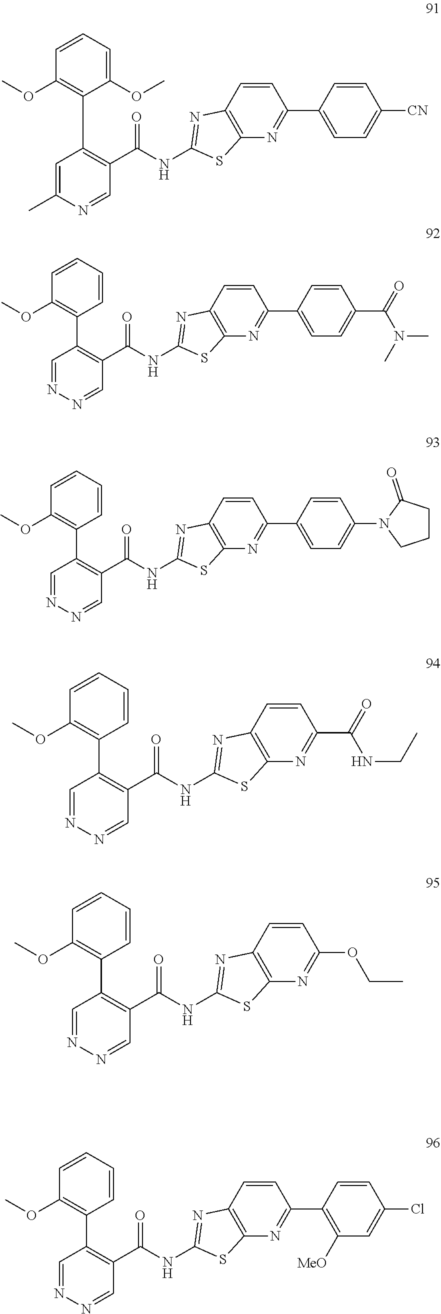

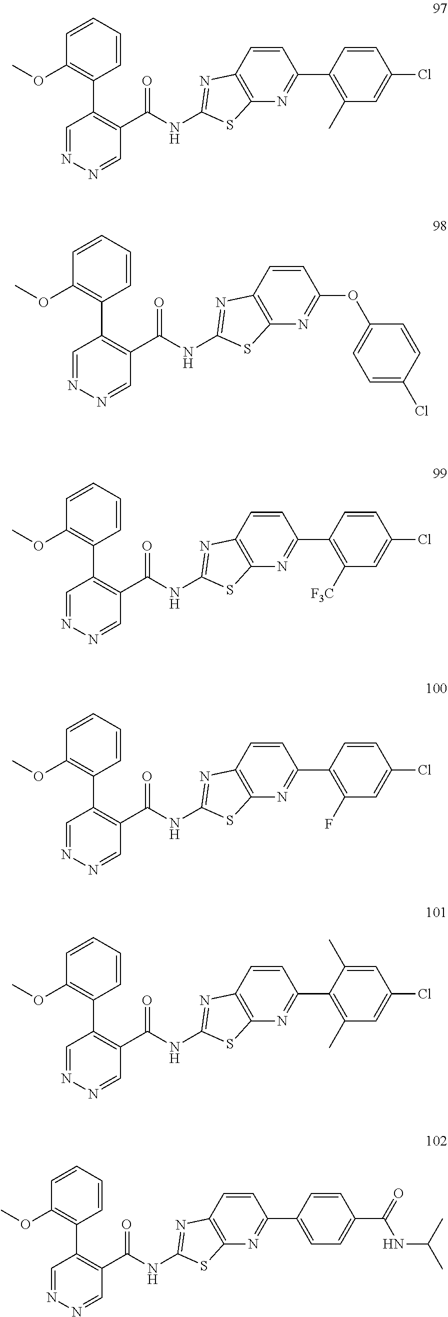 [SMILES: COc1ccccc1-c1cnncc1C(=O)Nc1nc2ccc(-c3ccc(Cl)cc3C)nc2s1 ; COc1ccccc1-c1cnncc1C(=O)Nc1nc2ccc(Oc3ccc(Cl)cc3)nc2s1 ; COc1ccccc1-c1cnncc1C(=O)Nc1nc2ccc(-c3ccc(Cl)cc3C(F)(F)F)nc2s1 ; COc1ccccc1-c1cnncc1C(=O)Nc1nc2ccc(-c3ccc(Cl)cc3F)nc2s1 ; COc1ccccc1-c1cnncc1C(=O)Nc1nc2ccc(-c3c(C)cc(Cl)cc3C)nc2s1 ; COc1ccccc1-c1cnncc1C(=O)Nc1nc2ccc(-c3ccc(C(=O)NC(C)C)cc3)nc2s1]

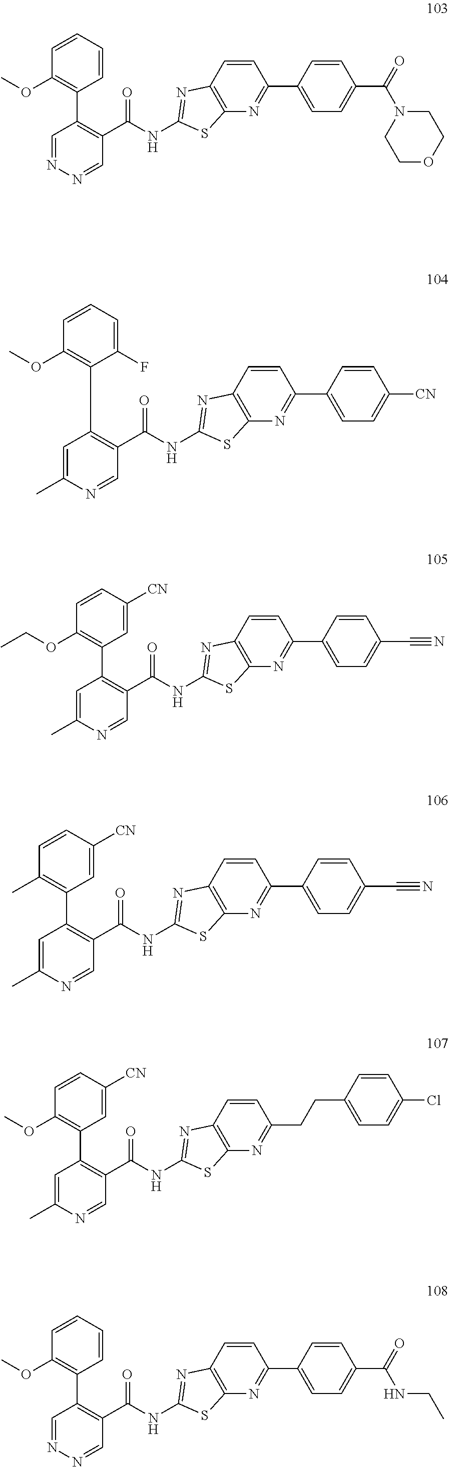 [SMILES: COc1ccccc1-c1cnncc1C(=O)Nc1nc2ccc(-c3ccc(C(=O)N4CCOCC4)cc3)nc2s1 ; COc1cccc(F)c1-c1cc(C)ncc1C(=O)Nc1nc2ccc(-c3ccc(C#N)cc3)nc2s1 ; CCOc1ccc(C#N)cc1-c1cc(C)ncc1C(=O)Nc1nc2ccc(-c3ccc(C#N)cc3)nc2s1 ; Cc1cc(-c2cc(C#N)ccc2C)c(C(=O)Nc2nc3ccc(-c4ccc(C#N)cc4)nc3s2)cn1 ; COc1ccc(C#N)cc1-c1cc(C)ncc1C(=O)Nc1nc2ccc(CCc3ccc(Cl)cc3)nc2s1 ; CCNC(=O)c1ccc(-c2ccc3nc(NC(=O)c4cnncc4-c4ccccc4OC)sc3n2)cc1]

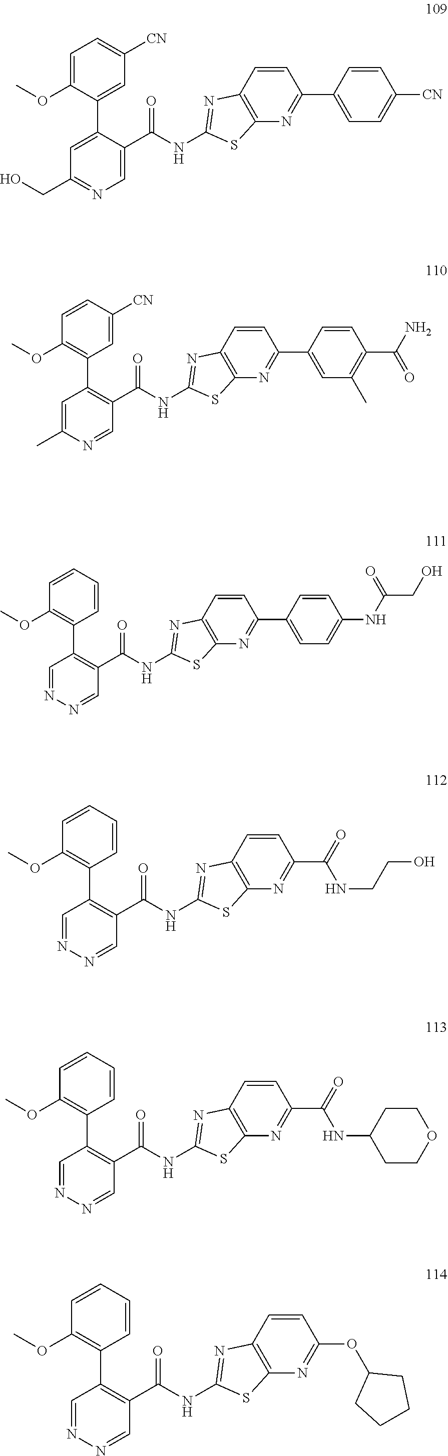 [SMILES: COc1ccc(C#N)cc1-c1cc(CO)ncc1C(=O)Nc1nc2ccc(-c3ccc(C#N)cc3)nc2s1 ; COc1ccc(C#N)cc1-c1cc(C)ncc1C(=O)Nc1nc2ccc(-c3ccc(C(N)=O)c(C)c3)nc2s1 ; COc1ccccc1-c1cnncc1C(=O)Nc1nc2ccc(-c3ccc(NC(=O)CO)cc3)nc2s1 ; COc1ccccc1-c1cnncc1C(=O)Nc1nc2ccc(C(=O)NCCO)nc2s1 ; COc1ccccc1-c1cnncc1C(=O)Nc1nc2ccc(C(=O)NC3CCOCC3)nc2s1 ; COc1ccccc1-c1cnncc1C(=O)Nc1nc2ccc(OC3CCCC3)nc2s1]

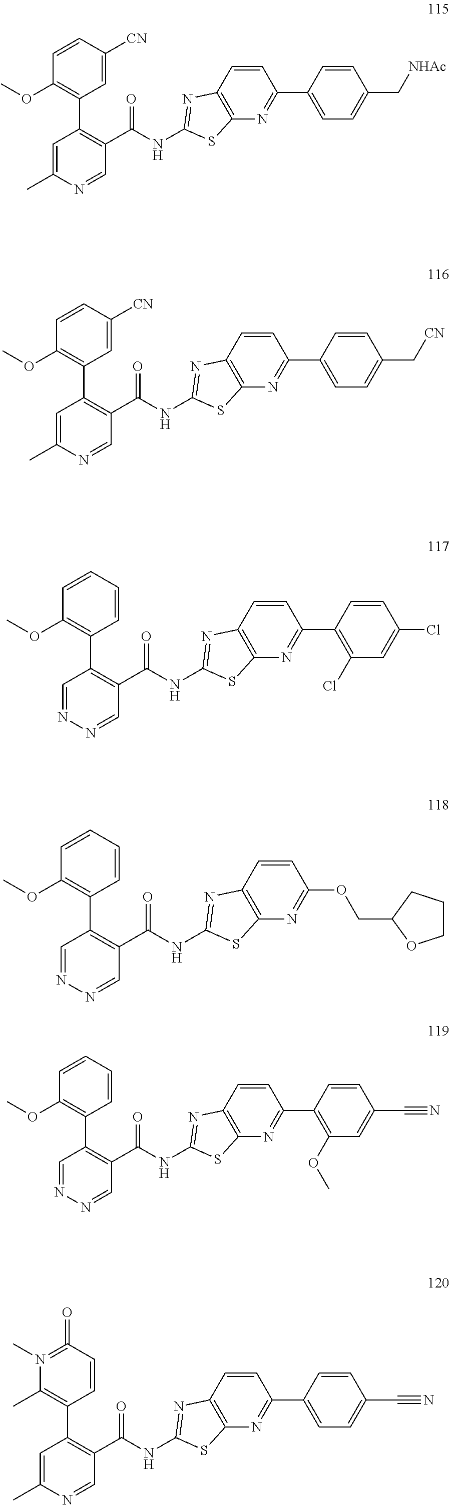

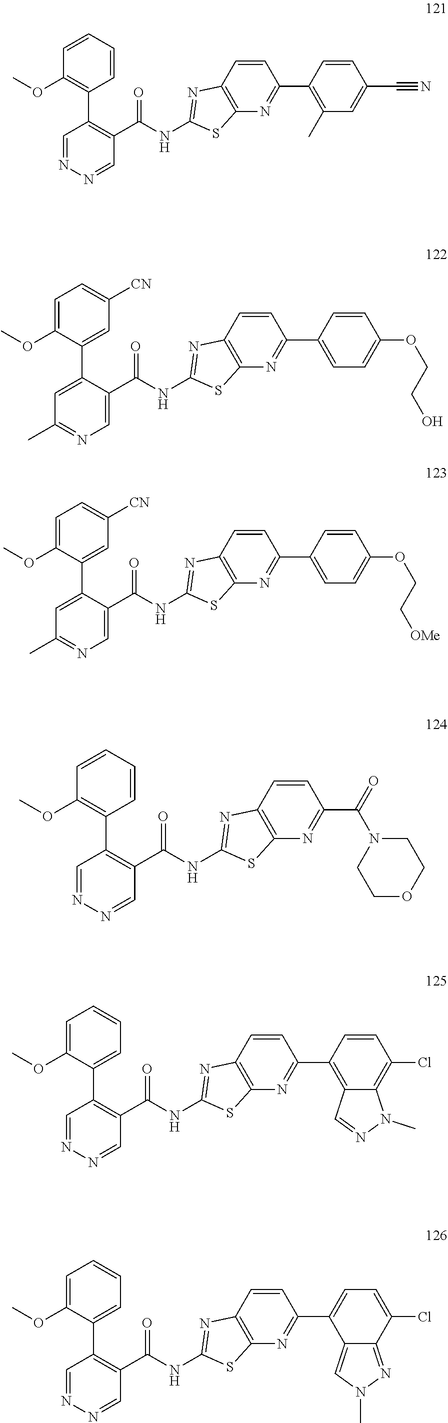 [SMILES: COc1ccccc1-c1cnncc1C(=O)Nc1nc2ccc(-c3ccc(C#N)cc3C)nc2s1 ; COc1ccc(C#N)cc1-c1cc(C)ncc1C(=O)Nc1nc2ccc(-c3ccc(OCCO)cc3)nc2s1 ; COCCOc1ccc(-c2ccc3nc(NC(=O)c4cnc(C)cc4-c4cc(C#N)ccc4OC)sc3n2)cc1 ; COc1ccccc1-c1cnncc1C(=O)Nc1nc2ccc(C(=O)N3CCOCC3)nc2s1 ; COc1ccccc1-c1cnncc1C(=O)Nc1nc2ccc(-c3ccc(Cl)c4c3cnn4C)nc2s1 ; COc1ccccc1-c1cnncc1C(=O)Nc1nc2ccc(-c3ccc(Cl)c4nn(C)cc34)nc2s1]

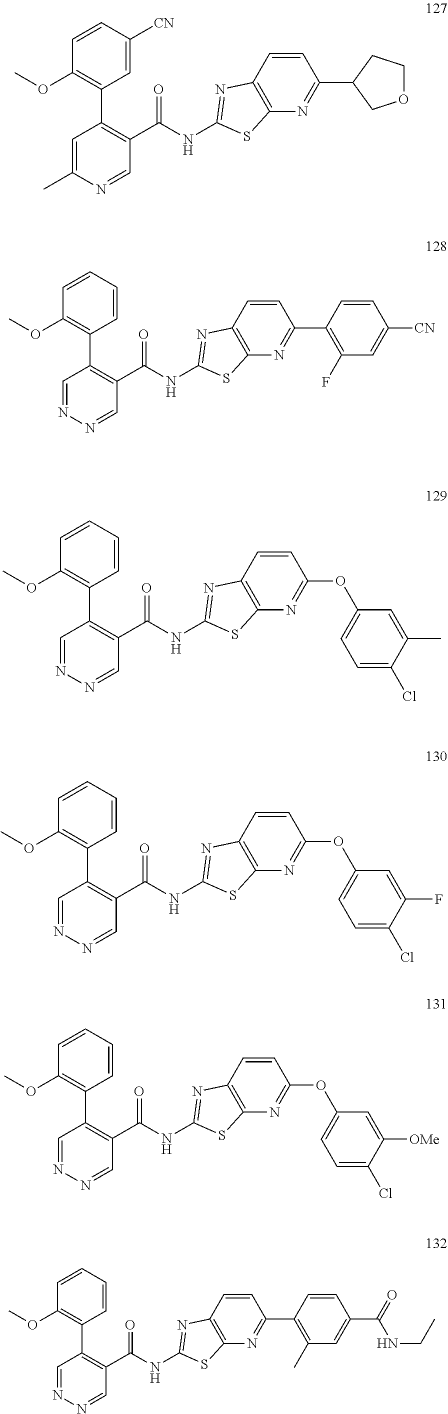

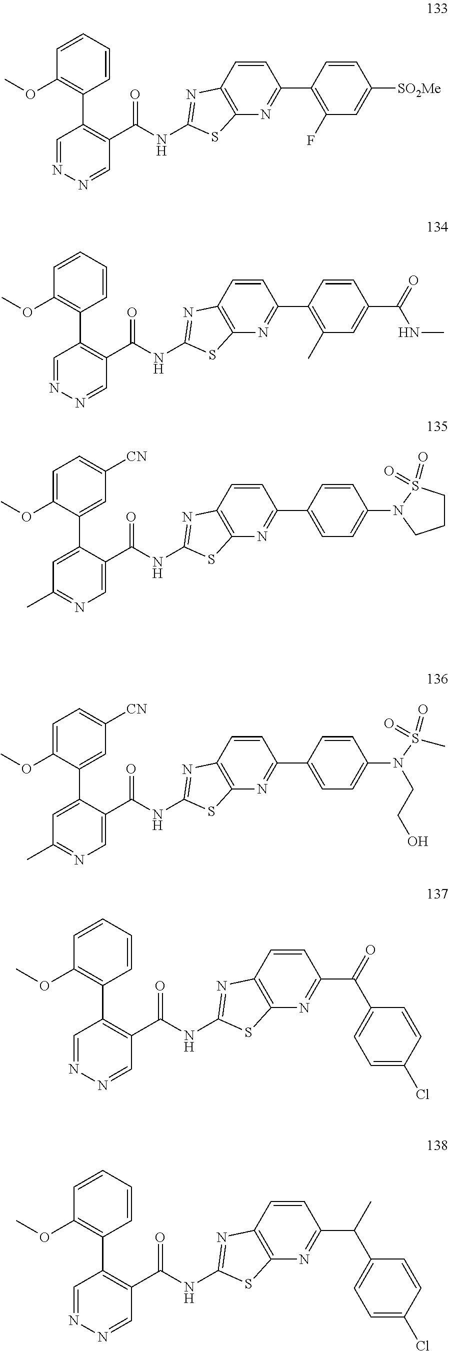

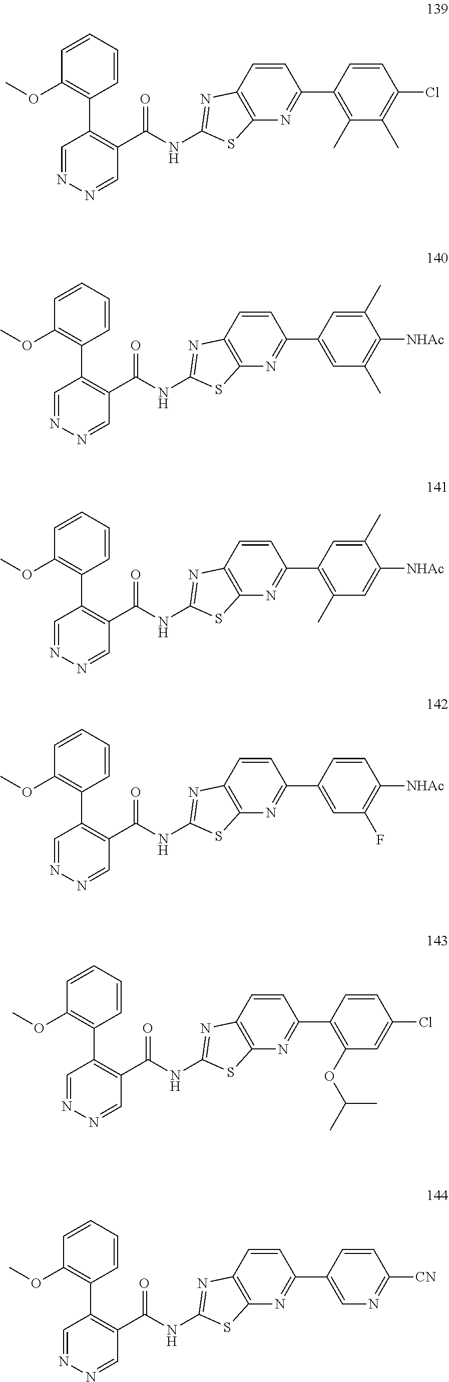 [SMILES: COc1ccccc1-c1cnncc1C(=O)Nc1nc2ccc(-c3ccc(Cl)c(C)c3C)nc2s1 ; COc1ccccc1-c1cnncc1C(=O)Nc1nc2ccc(-c3cc(C)c(NC(C)=O)c(C)c3)nc2s1 ; COc1ccccc1-c1cnncc1C(=O)Nc1nc2ccc(-c3cc(C)c(NC(C)=O)cc3C)nc2s1 ; COc1ccccc1-c1cnncc1C(=O)Nc1nc2ccc(-c3ccc(NC(C)=O)c(F)c3)nc2s1 ; COc1ccccc1-c1cnncc1C(=O)Nc1nc2ccc(-c3ccc(Cl)cc3OC(C)C)nc2s1 ; COc1ccccc1-c1cnncc1C(=O)Nc1nc2ccc(-c3ccc(C#N)nc3)nc2s1]

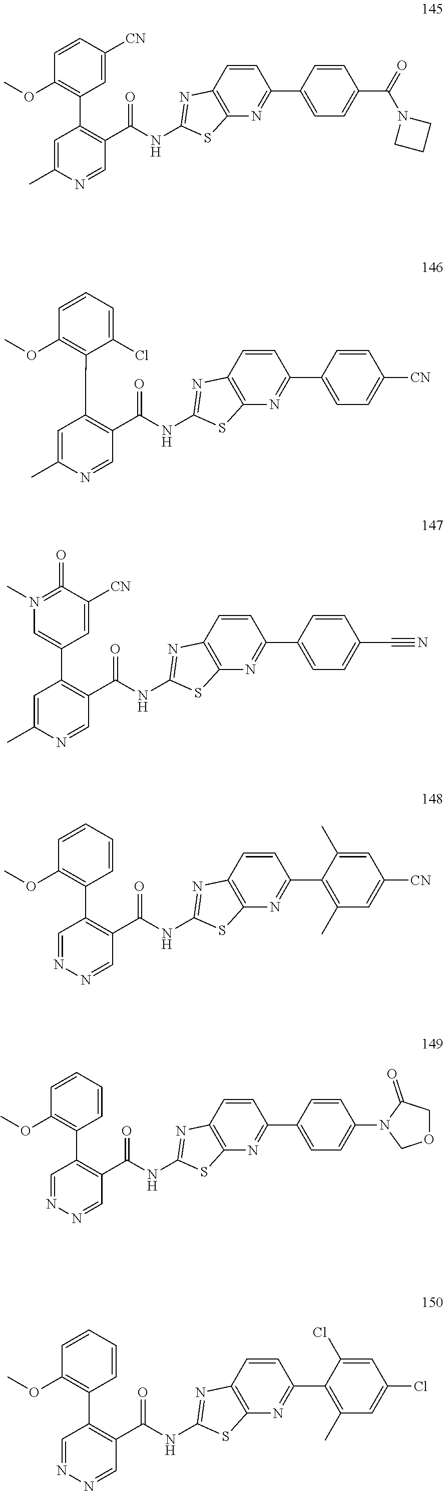

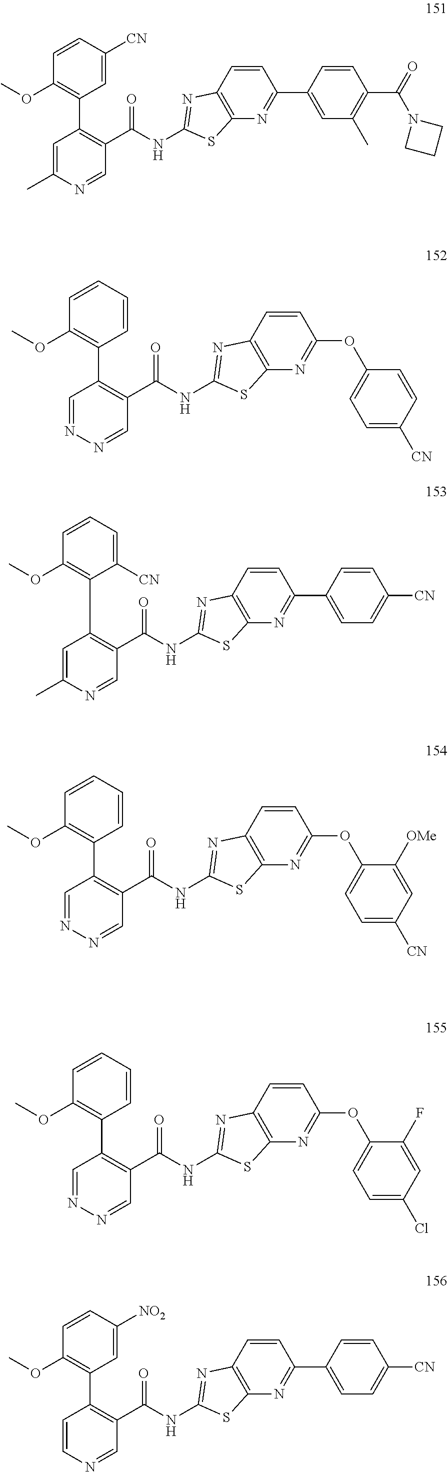 [SMILES: COc1ccc(C#N)cc1-c1cc(C)ncc1C(=O)Nc1nc2ccc(-c3ccc(C(=O)N4CCC4)c(C)c3)nc2s1 ; COc1ccccc1-c1cnncc1C(=O)Nc1nc2ccc(Oc3ccc(C#N)cc3)nc2s1 ; COc1cccc(C#N)c1-c1cc(C)ncc1C(=O)Nc1nc2ccc(-c3ccc(C#N)cc3)nc2s1 ; COc1cc(C#N)ccc1Oc1ccc2nc(NC(=O)c3cnncc3-c3ccccc3OC)sc2n1 ; COc1ccccc1-c1cnncc1C(=O)Nc1nc2ccc(Oc3ccc(Cl)cc3F)nc2s1 ; COc1ccc([N+](=O)[O-])cc1-c1ccncc1C(=O)Nc1nc2ccc(-c3ccc(C#N)cc3)nc2s1]

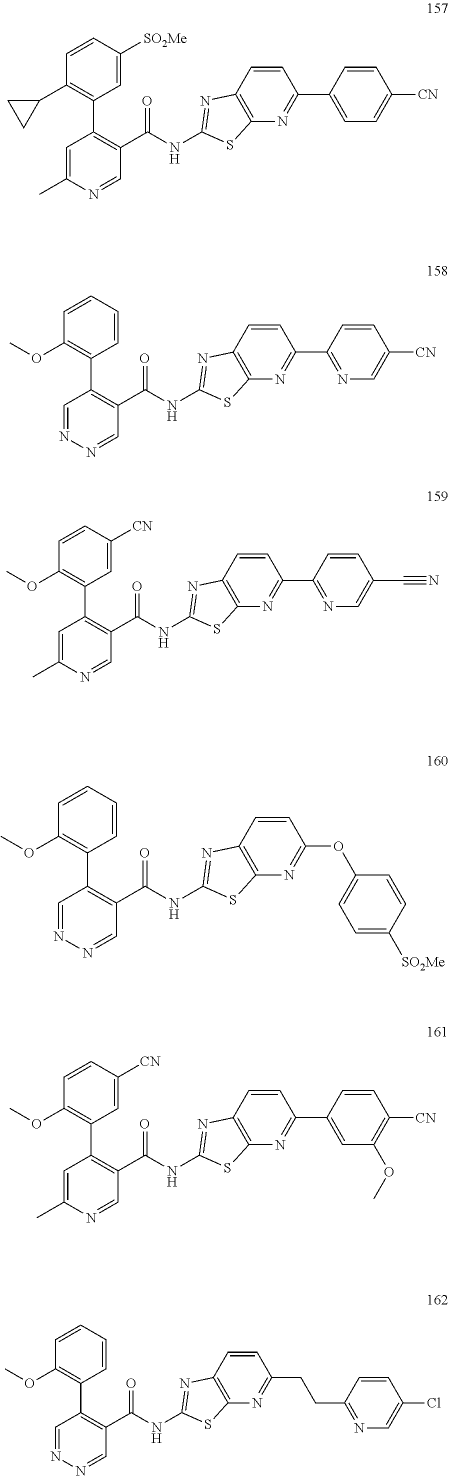 [SMILES: Cc1cc(-c2cc(S(C)(=O)=O)ccc2C2CC2)c(C(=O)Nc2nc3ccc(-c4ccc(C#N)cc4)nc3s2)cn1 ; COc1ccccc1-c1cnncc1C(=O)Nc1nc2ccc(-c3ccc(C#N)cn3)nc2s1 ; COc1ccc(C#N)cc1-c1cc(C)ncc1C(=O)Nc1nc2ccc(-c3ccc(C#N)cn3)nc2s1 ; COc1ccccc1-c1cnncc1C(=O)Nc1nc2ccc(Oc3ccc(S(C)(=O)=O)cc3)nc2s1 ; COc1cc(-c2ccc3nc(NC(=O)c4cnc(C)cc4-c4cc(C#N)ccc4OC)sc3n2)ccc1C#N ; COc1ccccc1-c1cnncc1C(=O)Nc1nc2ccc(CCc3ccc(Cl)cn3)nc2s1]

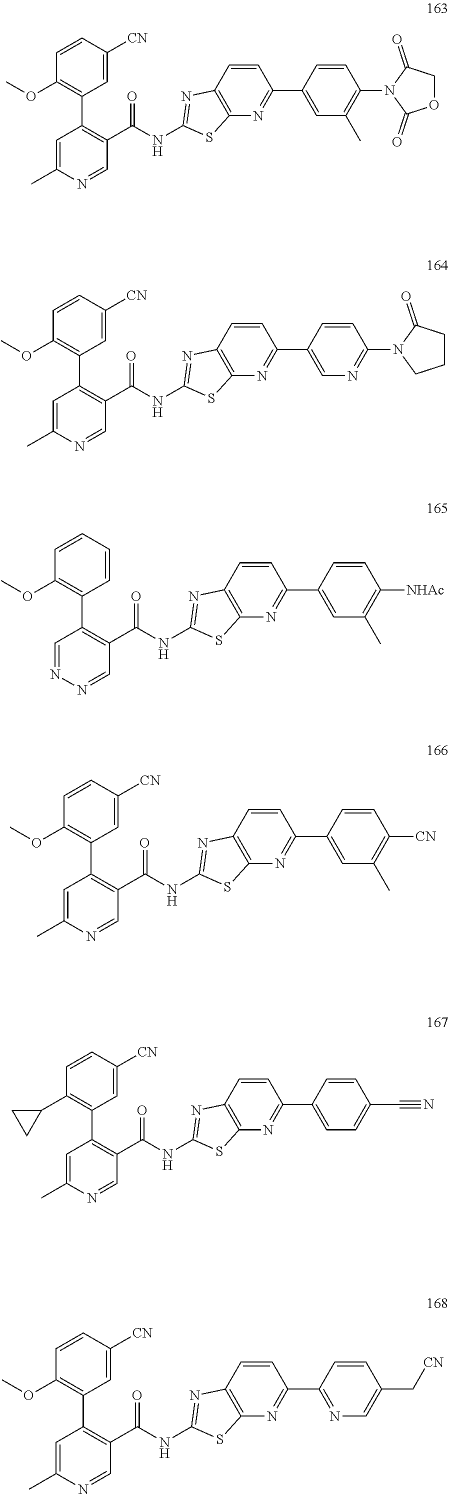 [SMILES: COc1ccc(C#N)cc1-c1cc(C)ncc1C(=O)Nc1nc2ccc(-c3ccc(N4C(=O)COC4=O)c(C)c3)nc2s1 ; COc1ccc(C#N)cc1-c1cc(C)ncc1C(=O)Nc1nc2ccc(-c3ccc(N4CCCC4=O)nc3)nc2s1 ; COc1ccccc1-c1cnncc1C(=O)Nc1nc2ccc(-c3ccc(NC(C)=O)c(C)c3)nc2s1 ; COc1ccc(C#N)cc1-c1cc(C)ncc1C(=O)Nc1nc2ccc(-c3ccc(C#N)c(C)c3)nc2s1 ; Cc1cc(-c2cc(C#N)ccc2C2CC2)c(C(=O)Nc2nc3ccc(-c4ccc(C#N)cc4)nc3s2)cn1 ; COc1ccc(C#N)cc1-c1cc(C)ncc1C(=O)Nc1nc2ccc(-c3ccc(CC#N)cn3)nc2s1]

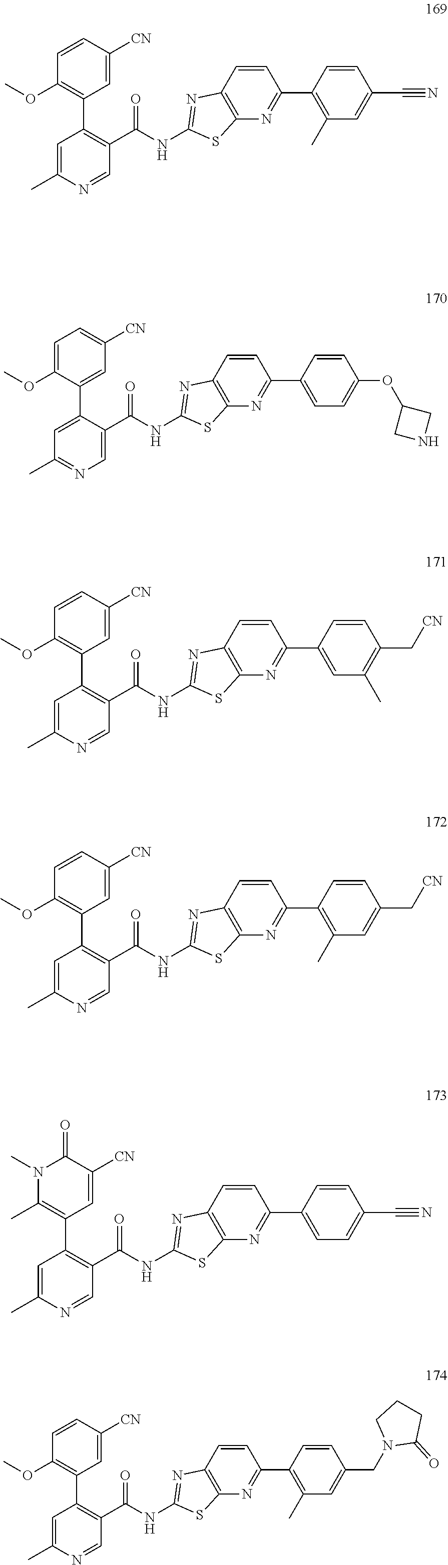 [SMILES: COc1ccc(C#N)cc1-c1cc(C)ncc1C(=O)Nc1nc2ccc(-c3ccc(C#N)cc3C)nc2s1 ; COc1ccc(C#N)cc1-c1cc(C)ncc1C(=O)Nc1nc2ccc(-c3ccc(OC4CNC4)cc3)nc2s1 ; COc1ccc(C#N)cc1-c1cc(C)ncc1C(=O)Nc1nc2ccc(-c3ccc(CC#N)c(C)c3)nc2s1 ; COc1ccc(C#N)cc1-c1cc(C)ncc1C(=O)Nc1nc2ccc(-c3ccc(CC#N)cc3C)nc2s1 ; Cc1cc(-c2cc(C#N)c(=O)n(C)c2C)c(C(=O)Nc2nc3ccc(-c4ccc(C#N)cc4)nc3s2)cn1 ; COc1ccc(C#N)cc1-c1cc(C)ncc1C(=O)Nc1nc2ccc(-c3ccc(CN4CCCC4=O)cc3C)nc2s1]

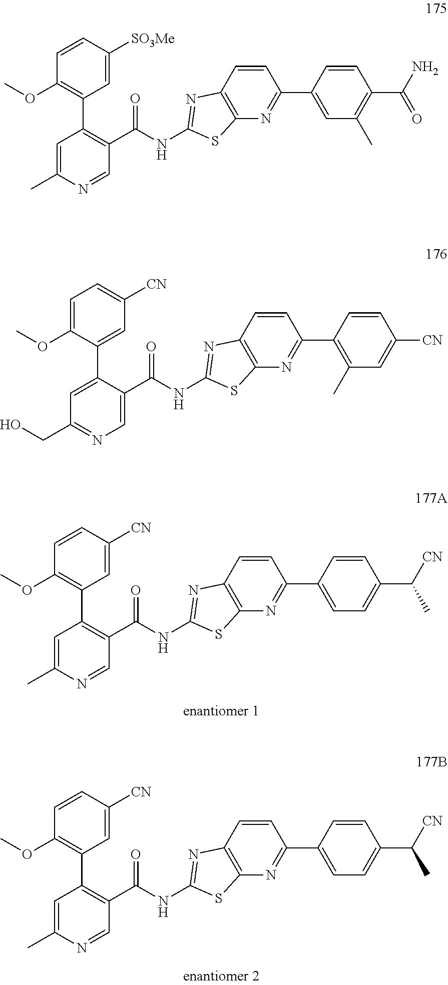

In the second aspect, disclosed herein is a pharmaceutical composition comprising the compound disclosed herein, or a pharmaceutically acceptable salt thereof, or the stereoisomer thereof; and at least one pharmaceutically acceptable carrier or excipient.

In the third aspect, disclosed herein is a method of inhibiting Pol Theta ATPase activity in a subject, which comprises administering to an individual compound disclosed herein, or a pharmaceutically acceptable salt thereof or the stereoisomer thereof, including the compound of formula (I) or the specific compounds exemplified herein; or disclosed herein is a use of an individual compound disclosed herein, or a pharmaceutically acceptable salt thereof or the stereoisomer thereof, including the compound of formula (I) or the specific compounds exemplified herein in the manufacture of a medicament for inhibiting Pol Theta ATPase activity in a subject; or disclosed here in an individual compound disclosed herein, or a pharmaceutically acceptable salt thereof or the stereoisomer thereof, including the compound of formula (I) or the specific compounds exemplified herein for use in inhibiting Pol Theta ATPase activity in a subject.

In the fourth aspect, disclosed herein is a method of treating a disease or disorder in a subject comprising administering to the subject in need thereof a therapeutically effective amount of the compound disclosed herein, or a pharmaceutically acceptable salt thereof or the stereoisomer thereof as a Pol Theta inhibitor, wherein the compound disclosed herein includes the compound of formula (I) or the specific compounds exemplified herein or disclosed herein is a use of an individual compound disclosed herein, or a pharmaceutically acceptable salt thereof or the stereoisomer thereof, including the compound of formula (I) or the specific compounds exemplified herein as a Pol Theta inhibitor in the manufacture of a medicament for treating a disease or disorder in a subject; or disclosed here in an individual compound disclosed herein, or a pharmaceutically acceptable salt thereof or the stereoisomer thereof, including the compound of formula (I) or the specific compounds exemplified herein as a Pol Theta inhibitor for use in treating a disease or disorder in a subject.

### DETAILED DESCRIPTION OF THE INVENTION

The following terms have the indicated meanings throughout the specification:

Unless specifically defined elsewhere in this document, all other technical and scientific terms used herein have the meaning commonly understood by one of ordinary skill in the art to which this invention belongs.

The following terms have the indicated meanings throughout the specification:

As used herein, including the appended claims, the singular forms of words such as “a”, “an”, and “the”, include their corresponding plural references unless the context clearly indicates otherwise.

The term “or” is used to mean, and is used interchangeably with, the term “and/or” unless the context clearly dictates otherwise.

The term “alkyl” includes a hydrocarbon group selected from linear and branched, saturated hydrocarbon groups comprising from 1 to 18, such as from 1 to 12, further such as from 1 to 10, more further such as from 1 to 8, or from 1 to 6, or from 1 to 4, carbon atoms. Examples of alkyl groups comprising from 1 to 6 carbon atoms (i.e., C₁₋₆ alkyl) include, but not limited to, methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups.

The term “propyl” includes 1-propyl or n-propyl (“n-Pr”), 2-propyl or isopropyl (“i-Pr”).

The term “butyl” includes 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”).

The term “pentyl” includes 1-pentyl, 2-pentyl, 3-pentyl, 2-methyl-2-butyl, 3-methyl-2-butyl, 3-methyl-1-butyl, 2-methyl-1-butyl.

The term “hexyl” includes 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl.

The term “alkylene” refers to a divalent alkyl group by removing two hydrogen from alkane. Alkylene includes but not limited to methylene, ethylene, propylene, and so on.

The term “halogen” includes fluoro (F), chloro (Cl), bromo (Br) and iodo (I).

The term “haloalkyl” refers to an alkyl group in which one or more hydrogen is/are replaced by one or more halogen atoms such as fluoro, chloro, bromo, and iodo. Examples of the haloalkyl include haloC₁₋₈ alkyl, haloC₁₋₆ alkyl or halo C₁₋₄ alkyl, but not limited to —CF₃, —CH₂Cl, —CH₂CF₃, —CHCl₂, CF₃, and the like.

The term “alkenyl” includes a hydrocarbon group selected from linear and branched hydrocarbon groups comprising at least one C═C double bond and from 2 to 18, such as from 2 to 8, further such as from 2 to 6, carbon atoms. Examples of the alkenyl group, e.g., C₂₋₆ alkenyl, include, but not limited to ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-dienyl, hex-1-enyl, hex-2-enyl, hex-3-enyl, hex-4-enyl, and hexa-1,3-dienyl groups.

The term “alkenylene” refers to a divalent alkenyl group by removing two hydrogen from alkene. Alkenylene includes but not limited to, vinylidene, butenylene, and so on.

The term “alkynyl” includes a hydrocarbon group selected from linear and branched hydrocarbon group, comprising at least one C≡C triple bond and from 2 to 18, such as 2 to 8, further such as from 2 to 6, carbon atoms. Examples of the alkynyl group, e.g., C₂₋₆ alkynyl, include, but not limited to ethynyl, 1-propynyl, 2-propynyl (propargyl), 1-butynyl, 2-butynyl, and 3-butynyl groups.

The term “alkynylene” refers to a divalent alkynyl group by removing two hydrogen from alkyne. Alkenylene includes but not limited to ethynylene and so on.

The term “alkyloxy” or “alkoxy” refers to an alkyl group as defined above attached to the parent molecular moiety through an oxygen atom. Examples of an alkyloxy, e.g., C1-6alkyloxy or C1-4 alkyloxy includes, but not limited to, methoxy, ethoxy, isopropoxy, propoxy, n-butoxy, tert-butoxy, pentoxy and hexoxy and the like.

The term “cycloalkyl” includes a hydrocarbon group selected from saturated cyclic hydrocarbon groups, comprising monocyclic and polycyclic (e.g., bicyclic and tricyclic) groups including fused, bridged or spiro cycloalkyl.

For example, the cycloalkyl group may comprise from 3 to 12, such as from 3 to 10, further such as 3 to 8, further such as 3 to 6, 3 to 5, or 3 to 4 carbon atoms. Even further for example, the cycloalkyl group may be selected from monocyclic group comprising from 3 to 12, such as from 3 to 10, further such as 3 to 8, 3 to 6 carbon atoms. Examples of the monocyclic cycloalkyl group include cyclopropyl, cyclobutyl, cyclopentyl, cyclohexyl, cycloheptyl, cyclooctyl, cyclononyl, cyclodecyl, cycloundecyl, and cyclododecyl groups.

In particular, examples of the saturated monocyclic cycloalkyl group, e.g., C₃₋₈cycloalkyl, include, but not limited to cyclopropyl, cyclobutyl, cyclopentyl, cyclohexyl, cycloheptyl, and cyclooctyl groups.

In a preferred embodiment, the cycloalkyl is a monocyclic ring comprising 3 to 6 carbon atoms (abbreviated as C₃₋₆ cycloalkyl), including but not limited to, cyclopropyl, cyclobutyl, cyclopentyl, and cyclohexyl. Examples of the bicyclic cycloalkyl groups include those having from 7 to 12 ring atoms arranged as a fused bicyclic ring selected from [4,4], [4,5], [5,5], [5,6] and [6,6] ring systems, or as a bridged bicyclic ring selected from bicyclo[2.2.1]heptane, bicyclo[2.2.2]octane, and bicyclo[3.2.2]nonane.

Further Examples of the bicyclic cycloalkyl groups include those arranged as a bicyclic ring selected from [5,6] and [6,6] ring systems, such as and wherein the wavy lines indicate the points of attachment.

The ring may be saturated or have at least one double bond (i.e. partially unsaturated), but is not fully conjugated, and is not aromatic, as aromatic is defined herein.

The term “spiro cycloalkyl” includes a cyclic structure which contains carbon atoms and is formed by at least two rings sharing one atom. “Heterocyclyl”, “heterocycle” or “heterocyclic” are interchangeable and refer to a non-aromatic heterocyclyl group comprising one or more heteroatoms selected from nitrogen, oxygen or optionally oxidized sulfur as ring members, with the remaining ring members being carbon, including monocyclic, fused, bridged, and spiro ring, i.e., containing monocyclic heterocyclyl, bridged heterocyclyl, spiro heterocyclyl, and fused heterocyclic groups.

Exemplary 3 to 8-membered heterocyclyl groups include, but not limited to, (as numbered from the linkage position assigned priority 1) pyrrolidin-1-yl, pyrrolidin-2-yl, pyrrolidin-3-yl, imidazolidin-2-yl, imidazolidin-4-yl, pyrazolidin-2-yl, pyrazolidin-3-yl, piperidin-1-yl, piperidin-2-yl, piperidin-3-yl, piperidin-4-yl, 2,5-piperazinyl, pyranyl, morpholinyl, morpholino, morpholin-2-yl, morpholin-3-yl, oxiranyl, aziridin-1-yl, aziridin-2-yl, azocan-1-yl, azocan-2-yl, azocan-3-yl, azocan-4-yl, azocan-5-yl, thiiranyl, azetidin-1-yl, azetidin-2-yl, azetidin-3-yl, oxetanyl, thietanyl, 1,2-dithietanyl, 1,3-dithietanyl, dihydropyridinyl, tetrahydropyridinyl, thiomorpholinyl, thioxanyl, piperazinyl, homopiperazinyl, homopiperidinyl, azepan-1-yl, azepan-2-yl, azepan-3-yl, azepan-4-yl, oxepanyl, thiepanyl, 1,4-oxathianyl, 1,4-dioxepanyl, 1,4-oxathiepanyl, 1,4-oxaazepanyl, 1,4-dithiepanyl, 1,4-thiazepanyl and 1,4-diazepanyl, 1,4-dithianyl, 1,4-azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, 1-pyrrolinyl, 2-pyrrolinyl, 3-pyrrolinyl, indolinyl, 2H-pyranyl, 4H-pyranyl, 1,4-dioxanyl, 1,3-dioxolanyl, pyrazolinyl, pyrazolidinyl, dithianyl, dithiolanyl, pyrazolidinyl, imidazolinyl, pyrimidinonyl, or 1,1-dioxo-thiomorpholinyl.

The term “fused cycloalkyl” includes a bicyclic cycloalkyl group as defined herein which is saturated and is formed by two or more rings sharing two adjacent atoms.

The term “bridged cycloalkyl” includes a cyclic structure which contains carbon atoms and is formed by two rings sharing two atoms which are not adjacent to each other. The term “7 to 10 membered bridged cycloalkyl” includes a cyclic structure which contains 7 to 12 carbon atoms and is formed by two rings sharing two atoms which are not adjacent to each other.

Examples of fused cycloalkyl, fused cycloalkenyl, or fused cycloalkynyl include but are not limited to bicyclo[1.1.0]butyl, bicyclo[2.1.0]pentyl, bicyclo[3.1.0]hexyl, bicyclo[4.1.0]heptyl, bicyclo[3.3.0]octyl, bicyclo[4.2.0]octyl, decalin, as well as benzo 3 to 8 membered cycloalkyl, benzo C₄ 6 cycloalkenyl, 2,3-dihydro-1H-indenyl, 1H-indenyl, 1, 2, 3,4-tetralyl, 1,4-dihydronaphthyl, etc. Preferred embodiments are 8 to 9 membered fused rings, which refer to cyclic structures containing 8 to 9 ring atoms within the above examples.

The term “aryl” used alone or in combination with other terms includes a group selected from:
(a) 5- and 6-membered carbocyclic aromatic rings, e.g., phenyl;
(b) bicyclic ring systems such as 7 to 12 membered bicyclic ring systems, wherein at least one ring is carbocyclic and aromatic, e.g., naphthyl and indanyl; and,
(c) tricyclic ring systems such as 10 to 15 membered tricyclic ring systems wherein at least one ring is carbocyclic and aromatic, e.g., fluorenyl.

The terms “aromatic hydrocarbon ring” and “aryl” are used interchangeably throughout the disclosure herein. In some embodiments, a monocyclic or bicyclic aromatic hydrocarbon ring has 5 to 10 ring-forming carbon atoms (i.e., C₅₋₁₀ aryl). Examples of a monocyclic or bicyclic aromatic hydrocarbon ring includes, but not limited to, phenyl, naphth-1-yl, naphth-2-yl, anthracenyl, phenanthrenyl, and the like. In some embodiments, the aromatic hydrocarbon ring is a naphthalene ring (naphth-1-yl or naphth-2-yl) or phenyl ring. In some embodiments, the aromatic hydrocarbon ring is a phenyl ring.

Specifically, the term “bicyclic fused aryl” includes a bicyclic aryl ring as defined herein. The typical bicyclic fused aryl is naphthalene.

The term “heteroaryl” and “heteroaromatic ring” are interchangeable and include a group selected from:
monocyclic rings (monocyclic heteroaryl group). The term “monocyclic heteroaryl” refers to ring is a monocyclic ring comprising 1 or 2 heteroatom ring members independently selected from nitrogen (N), sulfur (S) and oxygen (O). In some embodiments, the monocyclic or bicyclic aromatic heterocyclic ring is a 5- to 7-membered heteroaryl ring, which is monocyclic and which has 1 or 2 heteroatom ring members independently selected from nitrogen (N), sulfur (S) and oxygen (O).
bicyclic rings (bicyclic heteroaryl group). The term “bicyclic heteroaryl” refers to is an 8- to 12-membered heteroaryl ring, which is bicyclic and which has 1, 2, 3 or 4 heteroatom ring members independently selected from nitrogen, sulfur and oxygen.

Examples of monocyclic or bicyclic heteroaryl include, but are not limited to, (as numbered from the linkage position assigned priority 1) pyridyl (such as 2-pyridyl, 3-pyridyl, or 4-pyridyl), cinnolinyl, pyrazinyl, 2,4-pyrimidinyl, 3,5-pyrimidinyl, 2,4-imidazolyl, imidazopyridinyl, isoxazolyl, oxazolyl, thiazolyl, isothiazolyl, thiadiazolyl (such as 1,2,3-thiadiazolyl, 1,2,4-thiadiazolyl, or 1,3,4-thiadiazolyl), tetrazolyl, thienyl (such as thien-2-yl, thien-3-yl), triazinyl, benzothienyl, furyl or furanyl, benzofuryl, benzoimidazolyl, indolyl, isoindolyl, indolinyl, oxadiazolyl (such as 1,2,3-oxadiazolyl, 1,2,4-oxadiazolyl, or 1,3,4-oxadiazolyl), phthalazinyl, pyrazinyl, pyridazinyl, pyrrolyl, triazolyl (such as 1,2,3-triazolyl, 1,2,4-triazolyl, or 1,3,4-triazolyl), quinolinyl, isoquinolinyl, pyrazolyl, pyrrolopyridinyl (such as 1H-pyrrolo[2,3-b]pyridin-5-yl), pyrazolopyridinyl (such as 1H-pyrazolo[3,4-b]pyridin-5-yl), benzoxazolyl (such as benzo[d]oxazol-6-yl), pteridinyl, purinyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, furazanyl (such as furazan-2-yl, furazan-3-yl), benzofurazanyl, benzothiophenyl, benzothiazolyl, benzoxazolyl, quinazolinyl, quinoxalinyl, naphthyridinyl, furopyridinyl, benzothiazolyl (such as benzo[d]thiazol-6-yl), indazolyl (such as 1H-indazol-5-yl) and 5,6,7,8-tetrahydroisoquinoline.

The term “at least one substituent” disclosed herein includes, for example, from 1 to 4, such as from 1 to 3, further as 1 or 2, substituents, provided that the theory of valence is met. For example, “at least one substituent F” disclosed herein includes from 1 to 4, such as from 1 to 3, further as 1 or 2, substituents F.

The term “divalent” refers to a linking group capable of forming covalent bonds with two other moieties. For example, “a divalent cycloalkyl group” refers to a cycloalkyl group obtained by removing two hydrogen from the corresponding cycloalkane to form a linking group. the term “divalent aryl group”, “divalent heterocyclyl group” or “divalent heteroaryl group” should be understood in a similar manner.

Compounds disclosed herein may contain an asymmetric center and may thus exist as enantiomers. “Enantiomers” refer to two stereoisomers of a compound which are non-superimposable mirror images of one another. Where the compounds disclosed herein possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds disclosed herein and/or pharmaceutically acceptable salts thereof are intended to be included. Unless specifically mentioned otherwise, reference to one isomer applies to any of the possible isomers. Whenever the isomeric composition is unspecified, all possible isomers are included.

When compounds disclosed herein contain olefinic double bonds, unless specified otherwise, such double bonds are meant to include both E and Z geometric isomers.

When compounds disclosed herein contain a di-substituted cyclic ring system, substituents found on such ring system may adopt cis and trans formations. Cis formation means that both substituents are found on the upper side of the 2 substituent placements on the carbon, while trans would mean that they were on opposing sides. For example, the di-substituted cyclic ring system may be cyclohexyl or cyclobutyl ring.

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art could select and apply the techniques most likely to achieve the desired separation.

“Diastereomers” refer to stereoisomers of a compound with two or more chiral centers but which are not mirror images of one another. Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereomers using optically active resolving agents (Eliel, E. and Wilen, S. Stereochemistry of Organic Compounds. New York: John Wiley & Sons, Inc., 1994; Lochmuller, C. H., et al. “Chromatographic resolution of enantiomers: Selective review.”J. Chromatogr., 113(3) (1975): pp. 283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W., Ed. Drug Stereochemistry: Analytical Methods and Pharmacology. New York: Marcel Dekker, Inc., 1993.

Some of the compounds disclosed herein may exist with different points of attachment of hydrogen, referred to as tautomers. For example, compounds including carbonyl —CH₂C(O)— groups (keto forms) may undergo tautomerism to form hydroxyl —CH═C(OH)— groups (enol forms). Both keto and enol forms, individually as well as mixtures thereof, are also intended to be included where applicable. “Prodrug” refers to a derivative of an active agent that requires a transformation within the body to release the active agent. In some embodiments, the transformation is an enzymatic transformation.

Prodrugs are frequently, although not necessarily, pharmacologically inactive until converted to the active agent.

“Pharmaceutically acceptable salts” refer to those salts which are, within the scope of sound medical judgment, suitable for use in contact with the tissues of humans and lower animals without undue toxicity, irritation, allergic response and the like, and are commensurate with a reasonable benefit/risk ratio. A pharmaceutically acceptable salt may be prepared in situ during the final isolation and purification of the compounds disclosed herein, or separately by reacting the free base function with a suitable organic acid or by reacting the acidic group with a suitable base. The term also includes salts of the stereoisomers (such as enantiomers and/or diastereomers), tautomers and prodrugs of the compound of the invention.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

The terms “administration”, “administering”, “treating” and “treatment” herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, and rabbit) and most preferably a human.

The term “effective amount” or “therapeutically effective amount” refers to an amount of the active ingredient, such as compound that, when administered to a subject for treating a disease, or at least one of the clinical symptoms of a disease or disorder, is sufficient to affect such treatment for the disease, disorder, or symptom. The term “therapeutically effective amount” can vary with the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In some embodiments, “therapeutically effective amount” is an amount of at least one compound and/or at least one stereoisomer, tautomer or prodrug thereof, and/or at least one pharmaceutically acceptable salt thereof disclosed herein effective to “treat” as defined herein, a disease or disorder in a subject. In the case of combination therapy, the term “therapeutically effective amount” refers to the total amount of the combination objects for the effective treatment of a disease, a disorder or a condition.

The term “disease” refers to any disease, discomfort, illness, symptoms or indications, and can be interchangeable with the term “disorder” or “condition”.

Throughout this specification and the claims which follow, unless the context requires otherwise, the term “comprise”, and variations such as “comprises” and “comprising” are intended to specify the presence of the features thereafter, but do not exclude the presence or addition of one or more other features. When used herein the term “comprising” can be substituted with the term “containing”, “including” or sometimes “having”.

Throughout this specification and the claims which follow, the term “Cₙ₋ₘ” indicates a range which includes the endpoints, wherein n and m are integers and indicate the number of carbons. Examples include C₁₋₈, C₁₋₆, and the like.

Unless specifically defined elsewhere in this document, all other technical and scientific terms used herein have the meaning commonly understood by one of ordinary skill in the art to which this invention belongs.

### Therapeutic Utility

The compounds of the invention, subgroups and examples thereof, are inhibitors of DNA polymerase theta ATPase activity, which may be useful for the prevention and treatment of disease states or conditions described herein.

The compounds of the present invention may be useful for the treatment of the adult population, as well as the pediatric population.

It is anticipated that the compounds may be useful in diseases that relied on alt-NHEJ and MMEJ activities for survival, including but not limited to cancers and other disorders associated with dysfunction DNA repair or excessive cell accumulation.

It is expected this invention is typical for treatment of certain cancer. POLQ gene (DNA polymerase theta gene) is identified as the synthetic lethality of BRCA genes and other genes involved in HR pathway. In one embodiment, the compounds of this invention are useful for treatment of BRCA1 or BRCA2 deficient primary and secondary solid tumors, including but not limited to breast, ovarian, prostate and pancreas.

In a further embodiment, the DNA polymerase theta inhibitors are expected to show efficacy in tumor subtypes with HR-deficient genetic background other than BRCA1 and BRCA2. These genes include but not limit to ATR, RAD51, ATM, BARD1, RAD51C, CHEK1, CHEK2, FANCA, FANCB, FANCC, FANCD2, FANCE, FANCF, FANCG, FANCI, FANCL, FANCM, PALB2, FANCP, ERCC4, PTEN, CDK12, MRE11, NBS1, NBN, CLASPIN, BLM, WRN, RPA2 and BRIP1.

In a further embodiment, the compounds of this invention are useful to delay the onset of PARPi resistance based on the hypothesis that secondary BRCA revision mutation relies on the MMEJ pathway. Therefore, in one embodiment, the compounds of this invention have the potential to combine with PARPi in clinical practice, thus increasing the time to relapse of HRD primary and secondary tumors, including but not limited to breast, ovarian, prostate and pancreas.

In a further embodiment, the DNA polymerase theta inhibitors are expected to be used in the PARPi resistance cases (following or not following exposure to PARPi) with the mechanism-of-action of HR-pathway restoration. The mechanism-of-action includes but not limits to secondary BRCA reversion mutations, deficiency of 53BP1/shieldin complex and loss of RAD51c.

Since DNA polymerase theta is commonly considered as the backup pathway when c-NHEJ are compromised, the compounds of this invention are expected to show synthetic lethality in a variety of solid tumors with deficient c-NHEJ pathway. The deficiency refers to any genetic variation that leads to loss/deficiency of core effective proteins involved in c-NHEJ. In one embodiment, the genetic variation includes mutations, substitutions, insertions, deletions, and copy number variation (CNV). In another embodiment, c-NHEJ genes are selected from any of: LIG4, LIG3, POLL, POLM, PRKDC, XRCC4, XRCC5 and XRCC6.

DNA-damaging therapy, including radiotherapy and partial chemotherapy, kills cancer cells by induction of DNA damages following the cell apoptosis. DNA polymerase theta is associated with resistance to DNA-damaging therapy in certain cancer types, especially these subtypes with NHEJ deficiency. In a further embodiment, the compounds of this invention have the potential to sensitize a variety of cancer types to these conventional therapies.

In one embodiment, radiotherapy includes EBRT and brachytherapy and radioligand based therapies. This is useful to manage the NSCLC, HNSCC, rectal cancer, prostate cancer, and pancreatic cancer.

In one embodiment, chemotherapy includes topoisomerase inhibitors (e.g. etoposide, doxorubicin, hydroxycamptothecine, irinotecan). This is useful to manage the disease progression of SCLC, rectal cancer, CRC and STS.

In a further embodiment, the DNA polymerase theta inhibitors are expected to reduce the DNA replication stress response because DNA polymerase theta acts as the translesion synthesis polymerase (TLS). Therefore, the compounds of this invention could be used to sensitize the primary and secondary tumors (e.g. breast, ovarian, CRC, pancreas, esophagus, leukemias) to drugs that induce DNA replication stress (e.g. cisplatin, mitomycin, cyclophosphamide, cytarabine, hydroxyurea, gemcitabine). As well, any of the above combination therapy is expected to reduce the acquisition of drug-induced resistance and increase the time to relapse.

### EXAMPLES

Compounds disclosed herein, including salts thereof, can be prepared using known organic synthesis techniques and can be synthesized according to any of numerous possible synthetic routes.

The reaction for preparing compounds disclosed herein can be carried out in suitable solvents which can be readily selected by one of skill in the art of organic synthesis. Suitable solvents can be substantially non-reactive with the starting materials, the intermediates, or products at the temperatures at which the reactions are carried out, e.g., temperatures which can range from the solvent's boiling temperature. A given reaction can be carried out in one solvent or mixture of solvents.

The selection of appropriate protecting group, can be readily determined by one skilled in the art.

Reactions can be monitored according to any suitable method known in the art, such as NMR, UV, HPLC, LC-MS and TLC. Compounds can be purified by a variety of methods, including HPLC and normal phase silica chromatography.

Chiral analytic HPLC was used for the retention time analysis of different chiral examples, the conditions were divided into the methods as below according to the column, mobile phase, solvent ration used.

ABBREVIATIONS

| | |
|---|---|
| AcOK | potassium acetate |
| atm | atmosphere |
| BnBr | (bromomethyl)benzene |
| BTFFH | bis(tetramethylene)fluoroformamidinium |
| | hexafluorophosphate |
| DMSO | dimethyl sulfoxide |
| EDCI | 1-ethyl-3-(3-dimethylaminopropyl)carbodiimide |
| | hydrochloride |
| HATU | 1-[bis(dimethylamino)methylene]-1H-1,2,3- |
| | triazolo[4,5-b]pyridinium 3-oxid hexafluorophosphate |
| HOBt | hydroxybenzotriazole |
| LiHMDS | lithium bis(trimethylsilyl)amide |
| NBS | N-bromosuccinimide |
| NIS | N-iodosuccinimide |
| Pd(OAc)₂ | palladium acetate |
| Pd(PPh₃)₂Cl₂ | bis(triphenylphosphine)palladium(II) dichloride |
| Pd₂(dba)₃ | tris(dibenzylideneacetone)dipalladium(0) |
| PdCl₂[P(Cy)₃]₂ | dichlorobis(tricyclohexylphosphine)palladium(II) |
| prep-HPLC | preparative high performance liquid chromatography |
| prep-TLC | preparative thin layer chromatography |
| PyBOP | benzotriazole-1-yl-oxy-tris-pyrrolidino- |
| | phosphonium hexafluorophosphate |
| SEM | (2-(trimethylsilyl)ethoxy)methyl |
| T₃P | propanephosphonic acid anhydride |
| TBAF | tetrabutylammonium fluoride |
| TBS/TBDMS | tert-butyldimethylsilyl |
| TCFH | chloro-N,N,N′,N′-tetramethylformamidinium |
| | hexafluorophosphate |
| THF | tetrahydrofuran |
| THF | tetrahydrofuran |
| THP | tetrahydro-2H-pyran-2-yl |
| TLC | thin-layer chromatography |
| Tol | toluene |
| TsOH | p-toluenesulfonic acid |
| XantPhos | 4,5-bis(diphenylphosphino)-9,9-dimethylxanthene |
| XPhos | 2-dicyclohexylphosphino-2′,4′,6′-triisopropylbiphenyl |
| XPhos Pd G2 | chloro(2-dicyclohexylphosphino-2′,4′,6′-triisopropyl- |
| | 1,1′-biphenyl)[2-(2′-amino- |
| | 1,1′-biphenyl)]palladium(II) |
| XPhos Pd G3 | (2-dicyclohexylphosphino-2′,4′,6′-triisopropyl- |
| | 1,1′-biphenyl)[2-(2′-amino-1,1′- |
| | biphenyl)]palladium(II) methanesulfonate |

### Example 1: N-(5-(4-chlorophenyl)thiazolo[5,4-b]pyridin-2-yl)-6-cyano-2-morpholinonicotinamide

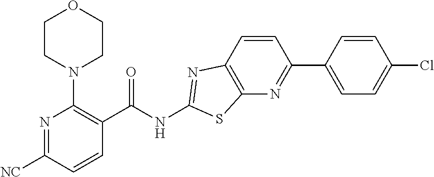 [SMILES: N#Cc1ccc(C(=O)Nc2nc3ccc(-c4ccc(Cl)cc4)nc3s2)c(N2CCOCC2)n1]

### Step 1: methyl 6-chloro-2-morpholinonicotinate

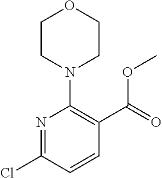 [SMILES: COC(=O)c1ccc(Cl)nc1N1CCOCC1]

To a solution of morpholine (1.69 g, 19.4 mmol) in N,N-Dimethylformamide (60 mL) was added dropwise methyl 2,6-dichloropyridine-3-carboxylate (3.8 g, 18.4 mmol) in N,N-dimethylformamide (20 mL) at room temperature. The resulting mixture was stirred for 1 h at room temperature under nitrogen atmosphere. After consumption of starting material, the reaction was quenched by the addition of water (50 mL). The resulting solution was extracted with ethyl acetate (3×70 mL). The organic phases were combined, washed with brine and dried over sodium sulfate. After filtration, the filtrate was concentrated under reduced pressure and the residue was purified over silica gel by flash chromatography eluting with ethyl acetate in petroleum ether (0% to 15% gradient, v/v) to yield the title compound (1.27 g, 27%). LC-MS (M+H)⁺=257.2.

### Step 2: methyl 6-cyano-2-morpholinonicotinate

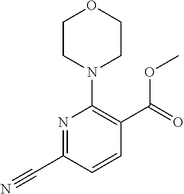 [SMILES: COC(=O)c1ccc(C#N)nc1N1CCOCC1]

To a solution of methyl 6-chloro-2-morpholinonicotinate (595 mg, 2.33 mmol) and Pd(PPh₃)₄ (512 mg, 0.444 mmol) in N,N-dimethylformamide (20 mL) was added Zn(CN)₂ (517 mg, 4.403 mmol) at room temperature. The resulting mixture was stirred for 3 h at 80° C. under a nitrogen atmosphere. After consumption of starting material, the reaction was then quenched by the addition of water (30 mL). The resulting mixture was extracted with ethyl acetate (3×50 mL). The organic phases were combined, washed with brine and dried over sodium sulfate. After filtration, the filtrate was concentrated under reduced pressure and the residue was purified over silica gel by flash chromatography eluting with ethyl acetate in petroleum ether (0% to 25% gradient, v/v) to yield the title compound (470 mg, 80%). LC-MS (M+H)⁺=248.2.

### Step 3: 6-cyano-2-morpholinonicotinic acid

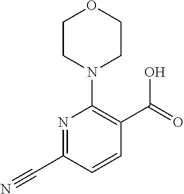 [SMILES: N#Cc1ccc(C(=O)O)c(N2CCOCC2)n1]

To a stirred solution of methyl 6-cyano-2-morpholinonicotinate (470 mg, 1.899 mmol) in THF (5 mL) was added LiOH (95 mg, 3.94 mmol) and water (2 mL) at room temperature. The resulting mixture was stirred for 5 h at room temperature under nitrogen atmosphere. When the reaction was completed, the resulting mixture was diluted with water (10 mL) and washed with ethyl acetate (3×10 mL). The aqueous layer was acidified to pH=5 with aqueous 2 N HCl solution. The precipitate was collected by filtration and washed with water (2×5 mL) to yield the title compound (303 mg, 68%). LC-MS (M+H)⁺=234.1.

### Step 4: tert-butyl (5-bromothiazolo[5,4-b]pyridin-2-yl)carbamate

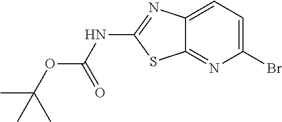 [SMILES: CC(C)(C)OC(=O)Nc1nc2ccc(Br)nc2s1]

A reaction mixture of 5-bromothiazolo[5,4-b]pyridin-2-amine (1 g, 4.35 mmol), di-tert-butyl dicarbonate (1.42 g, 6.52 mmol) and 4-dimethylaminopyridine (80 mg, 0.65 mmol) in dichloromethane (30 mL) was stirred at room temperate for 12 h. The mixture was concentrated to about 8 mL and the precipitate was filtered. The filtrate was purified over silica gel by combi-flash using methanol in dichloromethane (0% to 10%, v/v) to give the title compound (400 mg, 27%). LC-MS (M+H)⁺=330.0, 331.9.

### Step 5: tert-butyl (5-(4-chlorophenyl)thiazolo[5,4-b]pyridin-2-yl)carbamate

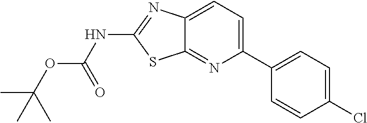 [SMILES: CC(C)(C)OC(=O)Nc1nc2ccc(-c3ccc(Cl)cc3)nc2s1]

A reaction mixture of tert-butyl (5-bromothiazolo[5,4-b]pyridin-2-yl)carbamate (400 mg, 1.21 mmol), (4-chlorophenyl)boronic acid (284 mg, 1.82 mmol), Pd(PPh₃)₄ (70 mg, 0.061 mmol) and K₂CO₃ (501 mg, 3.63 mmol) in 1,4-dioxane/water (35 mL, 6:1 v/v) under nitrogen was stirred at 100° C. for 12 h.

The solvent was evaporated and the residue was purified over silica gel by combi-flash, eluting with methanol in dichloromethane (0% to 10%, v/v) to give the title compound (180 mg, 41%). LC-MS (M+H)⁺=362.0.

### Step 6: 5-(4-chlorophenyl)thiazolo[5,4-b]pyridin-2-amine

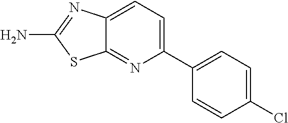 [SMILES: Nc1nc2ccc(-c3ccc(Cl)cc3)nc2s1]

To the reaction mixture of tert-butyl (5-(4-chlorophenyl)thiazolo[5,4-b]pyridin-2-yl)carbamate (180 mg, 0.5 mmol) in dichloromethane (3 mL) was added trifluoroacetic acid (3 mL) and stirred at room temperature for 2 h. The solvent was evaporated and the residue was treated with saturated aqueous solution of NaHCO₃. The precipitate was filtered, washed with water and dried to give the crude product (110 mg, 84%) which was used straightly in the next step without further purification. LC-MS (M+H)⁺=262.1.

### Step 7: N-(5-(4-chlorophenyl)thiazolo[5,4-b]pyridin-2-yl)-6-cyano-2-morpholinonicotinamide

To a mixture of 6-cyano-2-morpholinonicotinic acid (105 mg, 0.453 mmol) and O-(benzotriazol-1-yl)-N,N,N′,N′-tetramethyluronium tetrafluoroborate (219 mg, 0.681 mmol) in N,N-dimethylformamide (3 mL) was added N,N-diisopropylethylamine (237 uL, 1.361 mmol) dropwise at room temperature. The resulting mixture was stirred at 50° C. for 2 h under nitrogen atmosphere. To the above mixture were added 5-(4-chlorophenyl)thiazolo[5,4-b]pyridin-2-amine (118 mg, 0.454 mmol) in N,N-dimethylformamide (2 mL) dropwise at 50° C. under nitrogen atmosphere. The resulting mixture was stirred for 16 h at 80° C. under nitrogen atmosphere. After consumption of starting material, the resulting mixture was concentrated under reduced pressure. The resulting mixture was diluted with water (20 mL). The resulting mixture was extracted with ethyl acetate (3×15 mL). The organic phases were combined, washed with brine and dried over Na₂SO₄. After filtration, the filtrate was concentrated under reduced pressure and the residue was purified by prep-HPLC under the following conditions: column, XBridge Shield RP18 OBD Column, 30×150 mm, 5 um; mobile phase, acetonitrile in water (with 10 mmol/L NH₄HCO₃ and 0.1% NH₄OH), 28% to 58% gradient in 10 min; detector, UV 254 nm. The title compound (11 mg, 5%) was obtained. ¹H NMR (300 MHz, d6-DMSO) δ 13.21 (s, 1H), 8.33-7.98 (m, 5H), 7.65-7.43 (m, 3H), 3.70-3.61 (m, 4H), 3.41-3.32 (m, 4H). LC-MS (M+H)⁺=477.2.

### Example 2: N-(5-(4-chlorophenyl)thiazolo[5,4-b]pyridin-2-yl)-4-(2-methoxyphenyl)nicotinamide

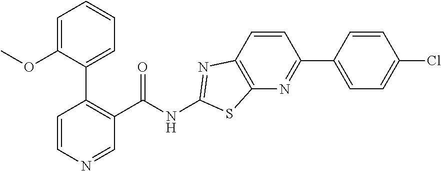 [SMILES: COc1ccccc1-c1ccncc1C(=O)Nc1nc2ccc(-c3ccc(Cl)cc3)nc2s1]

### Step 1: benzyl 4-chloronicotinate

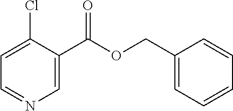 [SMILES: O=C(OCc1ccccc1)c1cnccc1Cl]

To a solution of 4-chloronicotinic acid (0.993 g, 6.3 mmol) in N,N-dimethylformamide (20 mL) was added (bromomethyl)benzene (1.2 g, 7.0 mmol) and K₂CO₃ (1.8 g, 12.7 mmol). The reaction mixture was stirred at 60° C. for 15 min under nitrogen atmosphere. The mixture was cooled to room temperature and diluted with ethyl acetate (50 mL), washed with brine (30 mL), dried over sodium sulfate, filtered and concentrated under reduce pressure to give a residue, which was purified over silica gel by combi-flash, eluting with ethyl acetate in petroleum ether (10%, v/v) to give the title compound (1.377 g, 88%). LC-MS (M+H)⁺=248.0.

### Step 2: benzyl 4-(2-methoxyphenyl)nicotinate

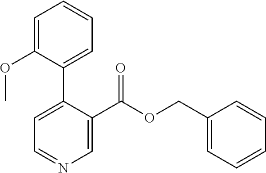 [SMILES: COc1ccccc1-c1ccncc1C(=O)OCc1ccccc1]

To a solution of benzyl 4-chloronicotinate (500 mg, 2.019 mmol) and (2-methoxyphenyl)boronic acid (613 mg, 4.037 mmol) in dioxane (18 mL) and water (3.6 mL) was added Pd(PPh₃)₄ (233 mg, 0.2019 mmol) and K₂CO₃ (561 mg, 4.037 mmol). The mixture was stirred at 80° C. for 2 h under nitrogen atomosphere. The reaction mixture was cooled to room temperature and diluted with ethyl acetate (30 mL). The solution was washed with brine (30 mL), dried with over sodium sulfate, decanted and concentrated under reduced pressure. The residue was purified over silica gel by combi-flash, eluting with ethyl acetate in petroleum ether (20%, v/v) to give the title compound (353 mg, 55%). LC-MS (M+H)⁺=320.1.

### Step 3: 4-(2-methoxyphenyl)nicotinic acid

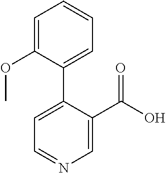 [SMILES: COc1ccccc1-c1ccncc1C(=O)O]

To a mixture of benzyl 4-(2-methoxyphenyl)nicotinate (353 mg, 1.106 mmol) in methanol (10 mL) was added 10 wt. % Pd/C (60 mg). The mixture was stirred under 1 atmosphere hydrogen at room temperature for 18 h. The mixture was filtered over celite and the filtrate was concentrated under reduced pressure to afford title compound (200 mg, 79%). LC-MS (M+H)⁺=230.1.

### Step 4: N-(5-(4-chlorophenyl)thiazolo[5,4-b]pyridin-2-yl)-4-(2-methoxyphenyl)nicotinamide

To a solution of 4-(2-methoxyphenyl)nicotinic acid (100 mg, 0.437 mmol) and 5-(4-chlorophenyl)thiazolo[5,4-b]pyridin-2-amine (100 mg, 0.383 mmol) in pyridine (3 mL) was added POCl₃ (110 mg, 0.874 mmol) at −10° C. under nitrogen. The mixture was stirred at 25° C. for 20 minutes under nitrogen. Saturated NaHCO₃ (5 mL) was added to quench the reaction, and the mixture was extracted with ethyl acetate (10 mL). The organic phase was washed with brine (10 mL) and dried with over sodium sulfate. After filtration, the filtrate was concentrated under reduced pressure. The residue was purified over silica gel by combi-flash, eluting with methanol in dichloromethane (5%, v/v) to give the title compound (11.9 mg, 7%). ¹H NMR (400 MHz, d6-DMSO) δ: 8.90 (s, 1H), 8.71 (s, 1H), 8.15 (d, J=8.5 Hz, 2H), 8.06-8.00 (m, 2H), 7.55 (d, J=8.5 Hz, 2H), 7.38 (dd, J=15.1, 7.2 Hz, 3H), 7.07 (t, J=7.4 Hz, 1H), 7.00 (d, J=8.2 Hz, 1H), 3.53 (s, 3H). LC-MS (M+H)⁺=473.1.

### Example 3: N-(5-(4-chlorophenyl)thiazolo[5,4-b]pyridin-2-yl)-3-(2-methoxyphenyl)isonicotinamide

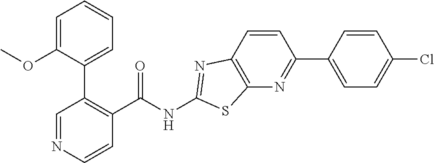 [SMILES: COc1ccccc1-c1cnccc1C(=O)Nc1nc2ccc(-c3ccc(Cl)cc3)nc2s1]

### Step 1: methyl 3-(2-methoxyphenyl)isonicotinate

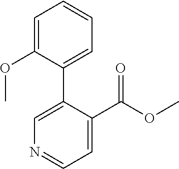 [SMILES: COC(=O)c1ccncc1-c1ccccc1OC]

The title compound (1.1 g, 97%) was prepared in a manner similar to Example 2 step 2 from methyl 3-bromoisonicotinate and (2-methoxyphenyl)boronic acid. ¹H NMR (400 MHz, d6-DMSO) δ 8.70 (d, J=5.0 Hz, 1H), 8.59 (s, 1H), 7.65 (d, J=5.0 Hz, 1H), 7.41 (td, J=7.9, 1.6 Hz, 1H), 7.33 (dd, J=7.4, 1.4 Hz, 1H), 7.07 (t, J=7.6 Hz, 2H), 3.66 (s, 3H), 3.66 (s, 3H). LC-MS (M+H)⁺=244.1.

### Step 2: 3-(2-methoxyphenyl)isonicotinic acid

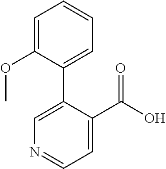 [SMILES: COc1ccccc1-c1cnccc1C(=O)O]

The title compound (700 mg, 67%) was prepared in a manner similar to Example 1 Step 3 from methyl 3-(2-methoxyphenyl)isonicotinate (1.1 g, 4.53 mmol). ¹H NMR (400 MHz, d6-DMSO) δ 13.10 (s, 1H), 8.66 (d, J=5.0 Hz, 1H), 8.53 (s, 1H), 7.63 (d, J=5.0 Hz, 1H), 7.50-7.32 (m, 1H), 7.29 (dd, J=7.5, 1.4 Hz, 1H), 7.04 (t, J=7.5 Hz, 2H), 3.67 (s, 3H). LC-MS (M+H)⁺=230.0.

### Step 3: N-(5-(4-chlorophenyl)thiazolo[5,4-b]pyridin-2-yl)-3-(2-methoxyphenyl)isonicotinamide

The reaction mixture of 3-(2-methoxyphenyl)isonicotinic acid (50 mg, 0.22 mmol), 5-(4-chlorophenyl)thiazolo[5,4-b]pyridin-2-amine (57 mg, 0.22 mmol), PyBOP (114 mg, 0.22 mmol) and N,N-diisopropylethylamine (56 mg, 0.44 mmol) in N,N-dimethylformamide (10 mL) was stirred at room temperature for 14 h. The solvent was evaporated and the residue was purified by prep-TLC using ethyl acetate as eluent to give the title compound (13 mg, 12%). ¹H NMR (400 MHz, d6-DMSO) δ 13.08 (s, 1H), 8.76 (d, J=4.9 Hz, 1H), 8.66 (s, 1H), 8.28-8.06 (m, 4H), 7.71 (d, J=4.9 Hz, 1H), 7.57 (d, J=8.6 Hz, 2H), 7.41 (dd, J=15.7, 7.6 Hz, 2H), 7.10 (t, J=7.4 Hz, 1H), 6.99 (d, J=8.2 Hz, 1H), 3.52 (s, 3H). LC-MS (M+H)⁺=473.0.

### Example 4: 6-cyano-2-morpholino-N-(5-(p-tolyl)thiazolo[5,4-b]pyridin-2-yl)nicotinamide

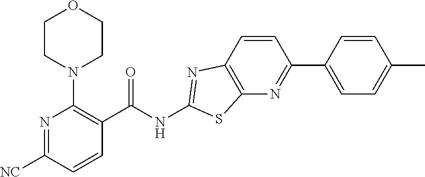 [SMILES: Cc1ccc(-c2ccc3nc(NC(=O)c4ccc(C#N)nc4N4CCOCC4)sc3n2)cc1]

### Step 1: 5-(p-tolyl)thiazolo[5,4-b]pyridin-2-amine

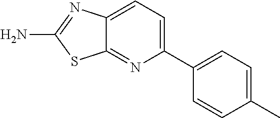 [SMILES: Cc1ccc(-c2ccc3nc(N)sc3n2)cc1]

The title compound (53 mg, 26%) was prepared in a manner similar to that in Example 1 step 5 from 5-bromothiazolo[5,4-b]pyridin-2-amine and p-tolylboronic acid. LC-MS (M+H)⁺=242.3.

### Step 2: 6-cyano-2-morpholino-N-(5-(p-tolyl)thiazolo[5,4-b]pyridin-2-yl)nicotinamide

To a solution of 5-(p-tolyl)thiazolo[5,4-b]pyridin-2-amine (42 mg, 0.17 mmol) and 6-cyano-2-morpholinonicotinic acid (42 mg, 0.18 mmol) in THF (2 mL) were added 1-methylimidazole (58 mg, 0.71 mmol) and TCFH (148 mg, 0.53 mmol) in portions at 0° C. The resulting mixture was stirred for 16 h at 70° C. under nitrogen atmosphere before cooled to room temperature and concentrated. The residue was purified by prep-HPLC under the following conditions: column, XBridge Shield RP18 OBD Column, 30×150 mm, 5 um; mobile phase, methanol in water (with 10 mmol/L NH₄HCO₃ and 0.1% NH₄OH), 20% to 50% gradient in 8 min; detector, UV 254 nm. The title compound (8 mg, 10%) was obtained. ¹H-NMR (400 MHz, d6-DMSO) δ 8.00 (d, J=6.8 Hz, 2H), 8.00-7.80 (m, 3H), 7.38 (d, J=7.6 Hz, 1H), 7.30 (d, J=7.6 Hz, 3.70-3.60 (m, 4H), 3.45-3.30 (m, 4H), 2.36 (s, 3H).

### Example 5: N-(5-(4-acetamidophenyl)thiazolo[5,4-b]pyridin-2-yl)-6-cyano-2-morpholinonicotinamide

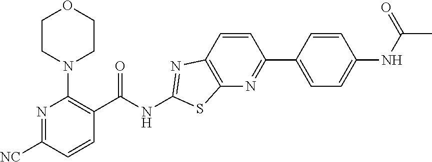 [SMILES: CC(=O)Nc1ccc(-c2ccc3nc(NC(=O)c4ccc(C#N)nc4N4CCOCC4)sc3n2)cc1]

### Step 1: tert-butyl (5-(4-aminophenyl)thiazolo[5,4-b]pyridin-2-yl)carbamate

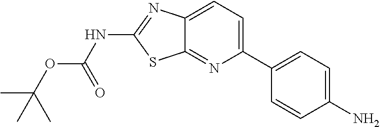 [SMILES: CC(C)(C)OC(=O)Nc1nc2ccc(-c3ccc(N)cc3)nc2s1]

The title compound (1.8 g, 91%) was prepared in a manner similar to Example 1 step 5 from tert-butyl (5-bromothiazolo[5,4-b]pyridin-2-yl)carbamate and (4-aminophenyl)boronic acid. LC-MS (M+H)⁺=343.1.

### Step 2: tert-butyl (5-(4-acetamidophenyl)thiazolo[5,4-b]pyridin-2-yl)carbamate

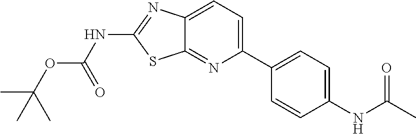 [SMILES: CC(=O)Nc1ccc(-c2ccc3nc(NC(=O)OC(C)(C)C)sc3n2)cc1]

Acetyl chloride (50 mg, 0.6 mmol) was added to a solution of tert-butyl (5-(4-aminophenyl)thiazolo[5,4-b]pyridin-2-yl)carbamate (100 mg, 0.3 mmol) and N,N-diisopropylethylamine (200 mg, 1.6 mmol) in dichloromethane (5 mL) dropwise at 0° C. The mixture was stirred at 0° C. for 15 min. The mixture was diluted with water, extracted with dichloromethane (3×20 mL). The combined organic layers were dried over Na₂SO₄, filtered and concentrated under vacuum. The residue was purified over silica gel by combi-flash, eluting with ethyl acetate in petroleum ether (50%, v/v) to give the title compound (100 mg, 87%). LC-MS (M+H)⁺=385.1.

### Step 3: N-(4-(2-aminothiazolo[5,4-b]pyridin-5-yl)phenyl)acetamide

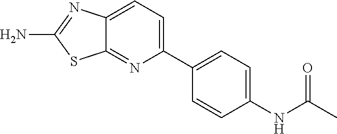 [SMILES: CC(=O)Nc1ccc(-c2ccc3nc(N)sc3n2)cc1]

The title compound (100 mg, crude) was prepared in a manner similar to that in Example 1 step 6 from tert-butyl (5-(4-acetamidophenyl)thiazolo[5,4-b]pyridin-2-yl)carbamate. LC-MS (M+H)⁺=285.1.

### Step 4: N-(5-(4-acetamidophenyl)thiazolo[5,4-b]pyridin-2-yl)-6-cyano-2-morpholinonicotinamide

The title compound (7 mg, 29%) was prepared in a manner similar to that in Example 4 step 2 from 6-cyano-2-morpholinonicotinic acid and N-(4-(2-aminothiazolo[5,4-b]pyridin-5-yl)phenyl)acetamide. 1H NMR (300 MHz, d6-DMSO) δ 13.10 (brs, 1H), 10.11 (s, 1H), 8.20-7.98 (m, 5H), 7.73 (d, J=8.7 Hz, 2H), 7.50 (d, J=7.5 Hz, 1H), 3.70-3.61 (m, 4H), 3.41-3.32 (m, 4H), 2.09 (s, 3H). LC-MS (M+H)⁺=500.2.

### Example 6: N-(5-(4-chlorophenyl)thiazolo[5,4-b]pyridin-2-yl)-6-cyano-2-(2-methoxyphenyl)nicotinamide

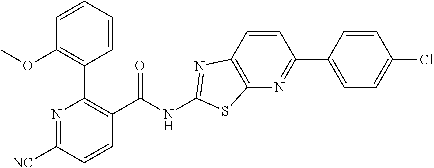 [SMILES: COc1ccccc1-c1nc(C#N)ccc1C(=O)Nc1nc2ccc(-c3ccc(Cl)cc3)nc2s1]

### Step 1: methyl 6-cyano-2-(2-methoxyphenyl)nicotinate

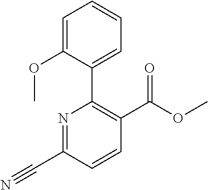 [SMILES: COC(=O)c1ccc(C#N)nc1-c1ccccc1OC]

The title compound (1040 mg, 81%) was prepared in a manner similar to that in Example 2 step 2 from methyl 2-chloro-6-cyanonicotinate and (2-methoxyphenyl)boronic acid. LC-MS (M+H)⁺=269.0.

### Step 2: 6-cyano-2-(2-methoxyphenyl)nicotinic acid

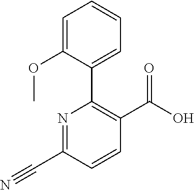 [SMILES: COc1ccccc1-c1nc(C#N)ccc1C(=O)O]

The title compound (300 mg, 33%) was prepared in a manner similar to that in Example 1 step 3 from methyl 6-cyano-2-(2-methoxyphenyl)nicotinate. LC-MS (M+H)⁺=255.1.

### Step 3: N-(5-(4-chlorophenyl)thiazolo[5,4-b]pyridin-2-yl)-6-cyano-2-(2-methoxyphenyl)nicotinamide

The title compound (30 mg, 15%) was prepared in a manner similar to that in Example 4 step 2 from 6-cyano-2-(2-methoxyphenyl)nicotinic acid and 5-(4-chlorophenyl)thiazolo[5,4-b]pyridin-2-amine. ¹H-NMR (400 MHz, d6-DMSO) δ 13.21 (s, 1H), 8.40 (d, J=8.0 Hz, 1H), 8.25-8.04 (m, 5H), 7.64-7.54 (m, 3H), 7.50-7.42 (m, 1H), 7.18-7.10 (m, 1H), 7.00 (d, J=8.3 Hz, 1H), 3.52 (s, 3H). LC-MS (M+H)⁺=498.1.

### Example 7: N-(5-(4-chlorophenyl)thiazolo[5,4-b]pyridin-2-yl)-6-cyano-2-(5-cyano-2-methoxyphenyl)nicotinamide

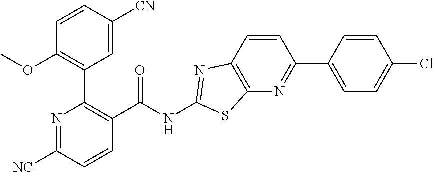 [SMILES: COc1ccc(C#N)cc1-c1nc(C#N)ccc1C(=O)Nc1nc2ccc(-c3ccc(Cl)cc3)nc2s1]

### Step 1: methyl 6-cyano-2-(5-cyano-2-methoxyphenyl)nicotinate

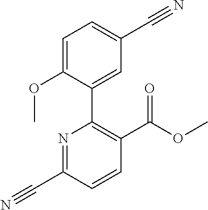 [SMILES: COC(=O)c1ccc(C#N)nc1-c1cc(C#N)ccc1OC]

The title compound (275 mg, 54%) was prepared in a manner similar to that in Example 2 step 2 from methyl 2-chloro-6-cyanonicotinate and (5-cyano-2-methoxyphenyl)boronic acid. LC-MS (M+H)⁺=294.0.

### Step 2: 6-cyano-2-(5-cyano-2-methoxyphenyl)nicotinic acid

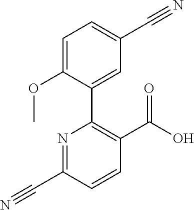 [SMILES: COc1ccc(C#N)cc1-c1nc(C#N)ccc1C(=O)O]

The title compound (169 mg, 77%) was prepared in a manner similar to that in Example 1 step 3 from methyl 6-cyano-2-(5-cyano-2-methoxyphenyl)nicotinate. LC-MS (M+H)⁺=280.2.

### Step 3: N-(5-(4-chlorophenyl)thiazolo[5,4-b]pyridin-2-yl)-6-cyano-2-(5-cyano-2-methoxyphenyl)nicotinamide

The title compound (37 mg, 14%) was prepared in a manner similar to that in Example 4 step 2 from 6-cyano-2-(5-cyano-2-methoxyphenyl)nicotinic acid and 5-(4-chlorophenyl)thiazolo[5,4-b]pyridin-2-amine. ¹H NMR (300 MHz, d6-DMSO) δ 13.28 (s, 1H), 8.47 (d, J=8.0 Hz, 1H), 8.28 (d, J=8.0 Hz, 1H), 8.23-8.12 (m, 3H), 8.09 (d, J=8.6 Hz, 1H), 8.05-7.92 (m, 2H), 7.62-7.51 (m, 2H), 7.21 (d, J=8.7 Hz, 1H), 3.61 (s, 3H). LC-MS (M+H)⁺=523.1.

### Example 8: N-(5-(4-chlorophenyl)thiazolo[5,4-b]pyridin-2-yl)-4-(5-cyano-2-methoxyphenyl)-6-methylnicotinamide

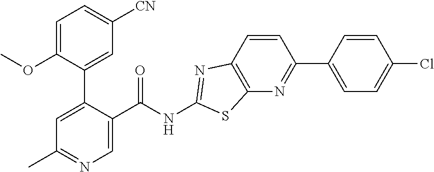 [SMILES: COc1ccc(C#N)cc1-c1cc(C)ncc1C(=O)Nc1nc2ccc(-c3ccc(Cl)cc3)nc2s1]

### Step 1: methyl 4-chloro-6-methylnicotinate

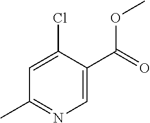

The title compound (3.7 g, 37%) was prepared in a manner similar to that in Example 2 step 1 from 4-chloro-6-methylnicotinic acid and iodomethane. ¹H NMR (400 MHz, d6-DMSO) δ 8.86 (s, 1H), 7.60 (s, 1H), 3.88 (s, 3H), 2.53 (s, 3H). LC-MS (M+H)⁺=186.0.

### Step 2: methyl 4-(5-cyano-2-methoxyphenyl)-6-methylnicotinate

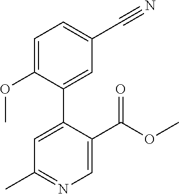

The title compound (2.7 g, 88.6%) was prepared in a manner similar to that in Example 2 step 2 from methyl 4-chloro-6-methylnicotinate and (5-cyano-2-methoxyphenyl)boronic acid. ¹H NMR (400 MHz, d6-DMSO) δ 8.82 (s, 1H), 7.92 (d, J=8.6 Hz, 1H), 7.80 (s, 1H), 7.34 (s, 1H), 7.25 (d, J=8.6 Hz, 1H), 3.75 (s, 3H), 3.65 (s, 3H), 2.56 (s, 3H). LC-MS (M+H)⁺=283.1.

### Step 3: 4-(5-cyano-2-methoxyphenyl)-6-methylnicotinic acid

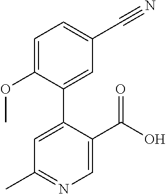 [SMILES: COc1ccc(C#N)cc1-c1cc(C)ncc1C(=O)O]

The title compound (2.2 g, 85.7%) was prepared in a manner similar to that in Example 1 step 3 from methyl 4-(5-cyano-2-methoxyphenyl)-6-methylnicotinate. ¹H NMR (400 MHz, d6-DMSO) δ 12.91 (s, 1H), 8.84 (s, 1H), 7.90 (dd, J=8.6, 2.0 Hz, 1H), 7.74 (d, J=2.0 Hz, 1H), 7.29 (s, 1H), 7.24 (d, J=8.7 Hz, 1H), 3.76 (s, 3H), 2.55 (s, 3H). LC-MS (M+H)⁺=269.1.

### Step 4: N-(5-(4-chlorophenyl)thiazolo[5,4-b]pyridin-2-yl)-4-(5-cyano-2-methoxyphenyl)-6-methylnicotinamide

The title compound (25 mg, 27%) was prepared in a manner similar to that described in Example 3 step 3 from 4-(5-cyano-2-methoxyphenyl)-6-methylnicotinic acid and 5-(4-chlorophenyl)thiazolo[5,4-b]pyridin-2-amine. ¹H-NMR (400 MHz, d6-DMSO) δ 13.05 (s, 1H), 8.81 (s, 1H), 8.23-8.09 (m, 4H), 7.95-7.90 (m, 2H), 7.57 (d, J=8.4 Hz, 2H), 7.47 (s, 1H), 7.19 (d, J=9.2 Hz, 1H), 3.60 (s, 3H), 2.61 (s, 3H). LC-MS (M+H)⁺=512.1.

### Example 9: N-(5-(4-chlorophenyl)thiazolo[5,4-b]pyridin-2-yl)-5-(2-methoxyphenyl)pyridazine-4-carboxamide

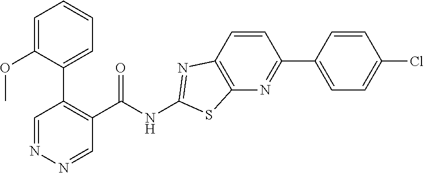 [SMILES: COc1ccccc1-c1cnncc1C(=O)Nc1nc2ccc(-c3ccc(Cl)cc3)nc2s1]

### Step 1: N,N-dimethylpyridazine-4-carboxamide

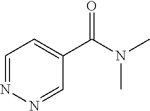

HATU (9 g, 24 mmol) was added to a solution of pyridazine-4-carboxylic acid (2.5 g, 20 mmol) and dimethylamine hydrochloride (3.2 g, 40 mmol) in Et₃N (10 mL) and N,N-dimethylformamide (30 mL). The mixture was stirred for 15 h at room temperature. The mixture was concentrated under vacuum. The residue was purified over silica gel by combi-flash, eluting with methanol in dichloromethane (1% to 2%, v/v) to give the title compound (2.0 g, 66%). LC-MS (M+H)⁺=152.1.

### Step 2: 5-(2-methoxyphenyl)-N,N-dimethylpyridazine-4-carboxamide

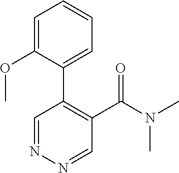 [SMILES: COc1ccccc1-c1cnncc1C(=O)N(C)C]

(2-Methoxyphenyl)magnesium bromide (16 ml, 16 mmol) was added to a solution of N,N-dimethylpyridazine-4-carboxamide (2.0 g, 13.2 mmol) in anhydrous THF (30 mL) dropwise at 0° C. The mixture was stirred at room temperature for 15 h. The mixture was concentrated under vacuum. The residue was diluted with ethyl acetate (100 mL). To the mixture was added iodobenzene diacetate (7.4 g, 23 mmol). The mixture was stirred at room temperature for 3 h. The mixture was quenched with NaHSO₃ solution, extracted with ethyl acetate (2×50 mL). The combined organic layers were washed with brine, dried over sodium sulfate, filtered and concentrated under vacuum. The residue was purified over silica gel by combi-flash, eluting with methanol in dichloromethane (1% to 5%, v/v) to give the title compound (2.0 g, 59%). LC-MS (M+H)⁺=258.1.

### Step 3: 5-(2-methoxyphenyl)pyridazine-4-carboxylic acid

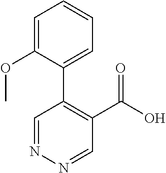 [SMILES: COc1ccccc1-c1cnncc1C(=O)O]

To a solution of 5-(2-methoxyphenyl)-N,N-dimethylpyridazine-4-carboxamide (2.0 g, 7.8 mmol) in EtOH (20 mL) was added a solution of KOH (2.2 g, 39 mmol) in water (20 mL). The mixture was stirred at 100° C. for 3 days. The mixture was cooled and acidified with aqueous 2M HCl solution to pH=2. The mixture was concentrated under vacuum to remove most of solvent. The residue was cooled and filtered. The filter cake was dried under vacuum to give title compound (1.0 g, 55%). LC-MS (M+H)⁺=231.0.

### Step 4: N-(5-(4-chlorophenyl)thiazolo[5,4-b]pyridin-2-yl)-5-(2-methoxyphenyl)pyridazine-4-carboxamide

Oxalyl dichloride (200 mg, 1.6 mmol) was added dropwise to a suspension of 5-(2-methoxyphenyl)pyridazine-4-carboxylic acid (50 mg, 0.22 mmol) in dichloromethane (1 mL) at 0° C. To the mixture was added a drop of N,N-dimethylformamide and the resulting mixture was stirred at 0° C. for 30 min before concentrated under vacuum. To the residue at 0° C. was added a solution of 5-(4-chlorophenyl)thiazolo[5,4-b]pyridin-2-amine (57 mg, 0.22 mmol) in pyridine (1 mL). The mixture was stirred at room temperature for 3 h before quenched with water. The mixture was extracted with ethyl acetate (3×10 mL) and the combined organic layers were washed with brine, dried over Na₂SO₄, filtered, then concentrated under vacuum. The residue was purified by prep-TLC using ethyl acetate as eluant to give title compound (30 mg, 29%). ¹H NMR (400 MHz, d6-DMSO) δ 13.32 (s, 1H), 9.50 (s, 1H), 9.42 (s, 1H), 8.25-8.15 (m, 3H), 8.12 (d, J=8.7 Hz, 1H), 7.62-7.45 (m, 4H), 7.19-7.12 (m, 1H), 7.07 (d, J=8.3 Hz, 1H), 3.56 (s, 3H). LC-MS (M+H)⁺=474.0, 476.0.

### Example 10: N-(5-(4-chlorophenyl)thiazolo[5,4-b]pyridin-2-yl)-6-cyano-2-(2-fluoro-6-methoxyphenyl)nicotinamide

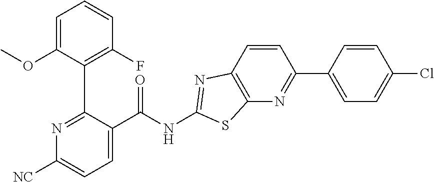 [SMILES: COc1cccc(F)c1-c1nc(C#N)ccc1C(=O)Nc1nc2ccc(-c3ccc(Cl)cc3)nc2s1]

### Step 1: methyl 6-cyano-2-(2-fluoro-6-methoxyphenyl)nicotinate

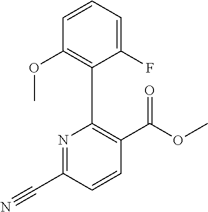 [SMILES: COC(=O)c1ccc(C#N)nc1-c1c(F)cccc1OC]

The title compound (200 mg, 40%) was prepared in a manner similar to that in Example 2 step 2 from methyl 2-chloro-6-cyanonicotinate and (2-fluoro-6-methoxyphenyl)boronic acid. LC-MS (M+H)⁺=287.1.

### Step 2: 6-cyano-2-(2-fluoro-6-methoxyphenyl)nicotinic acid

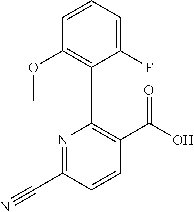 [SMILES: COc1cccc(F)c1-c1nc(C#N)ccc1C(=O)O]

The title compound (116 mg, 82%) was prepared in a manner similar to that in Example 1 step 3 from methyl 6-cyano-2-(2-fluoro-6-methoxyphenyl)nicotinate. LC-MS (M+H)⁺=273.0.

### Step 3: N-(5-(4-chlorophenyl)thiazolo[5,4-b]pyridin-2-yl)-6-cyano-2-(2-fluoro-6-methoxyphenyl)nicotinamide

The title compound (51 mg, 28%) was prepared in a manner similar to that in Example 4 step 2 from 6-cyano-2-(2-fluoro-6-methoxyphenyl)nicotinic acid and 5-(4-chlorophenyl)thiazolo[5,4-b]pyridin-2-amine. ¹H NMR (400 MHz, d6-DMSO) δ 13.36 (brs, 1H), 8.51 (d, J=8.0 Hz, 1H), 8.30 (d, J=8.0 Hz, 1H), 8.25-8.09 (m, 4H), 7.61-7.54 (m, 2H), 7.52-7.42 (m, 1H), 7.01-6.86 (m, 2H), 3.58 (s, 3H). LC-MS (M+H)⁺=516.0.

### Example 11: N-(5-(4-chlorophenyl)thiazolo[5,4-b]pyridin-2-yl)-3-(5-cyano-2-methoxyphenyl)isonicotinamide

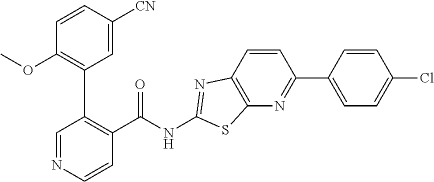 [SMILES: COc1ccc(C#N)cc1-c1cnccc1C(=O)Nc1nc2ccc(-c3ccc(Cl)cc3)nc2s1]

### Step 1: methyl 3-(5-cyano-2-methoxyphenyl)isonicotinate

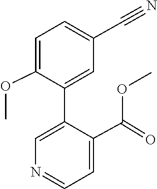 [SMILES: COC(=O)c1ccncc1-c1cc(C#N)ccc1OC]

The title compound (670 mg, 94%) was prepared in a manner similar to Example 2 step 2 from methyl 3-bromoisonicotinate (570 g, 2.64 mmol) and (5-cyano-2-methoxyphenyl)boronic acid (560 mg, 3.61 mmol). ¹H NMR (400 MHz, d6-DMSO) δ 8.77 (d, J=5.0 Hz, 1H), 8.65 (s, 1H), 7.93 (dd, J=8.6, 2.0 Hz, 1H), 7.87 (d, J=2.0 Hz, 1H), 7.72 (d, J=5.0 Hz, 1H), 7.25 (d, J=8.6 Hz, 1H), 3.76 (s, 3H), 3.68 (s, 3H). LC-MS (M+H)⁺=269.1.

### Step 2: 3-(5-cyano-2-methoxyphenyl)isonicotinic acid

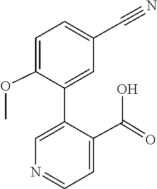 [SMILES: COc1ccc(C#N)cc1-c1cnccc1C(=O)O]

The title compound (470 mg, 74%) was prepared in a manner similar to Example 1 Step 3 from methyl 3-(5-cyano-2-methoxyphenyl)isonicotinate (670 mg, 2.5 mmol). ¹H NMR (400 MHz, d6-DMSO) δ 13.32 (s, 1H), 8.73 (d, J=5.0 Hz, 1H), 8.58 (s, 1H), 7.89 (dd, J=8.6, 2.1 Hz, 1H), 7.80 (d, J=2.1 Hz, 1H), 7.72-7.65 (m, 1H), 7.25 (d, J=8.7 Hz, 1H), 3.74 (d, J=12.0 Hz, 3H). LC-MS (M+H)⁺=255.0.

### Step 3: N-(5-(4-chlorophenyl)thiazolo[5,4-b]pyridin-2-yl)-3-(5-cyano-2-methoxyphenyl)isonicotinamide

The reaction mixture of 3-(5-cyano-2-methoxyphenyl)isonicotinic acid (50 mg, 0.2 mmol), 5-(4-chlorophenyl)thiazolo[5,4-b]pyridin-2-amine (50 mg, 0.2 mmol), T₃P (50% in ethyl acetate, 1.5 mL) and pyridine (1.5 mL) under nitrogen was stirred at 60° C. for 14 h. The solvent was evaporated and the residue was treated with a saturated aqueous solution of NaHCO₃. The mixture was extracted with ethyl acetate (2×10 mL). The organic layers were combined, washed with brine, dried over Na₂SO₄, filtered and evaporated. The residue was purified by prep-TLC using ethyl acetate as eluant and prep-HPLC (Elution: acetonitrile (0.1% formic acid)/water (0.1% FA): 50%-80%, v/v) to give the title compound (27 mg, 27%). ¹H NMR (400 MHz, d6-DMSO) δ 13.16 (s, 1H), 8.83 (d, J=4.9 Hz, 1H), 8.72 (s, 1H), 8.36-8.05 (m, 4H), 8.00-7.88 (m, 2H), 7.77 (d, J=5.0 Hz, 1H), 7.57 (d, J=8.6 Hz, 2H), 7.18 (d, J=8.6 Hz, 1H), 3.60 (s, 3H). LC-MS (M+H)⁺=498.1.

### Example 12: N-(5-(4-chlorophenyl)thiazolo[5,4-b]pyridin-2-yl)-1-(2-methoxyphenyl)-1H-imidazole-5-carboxamide

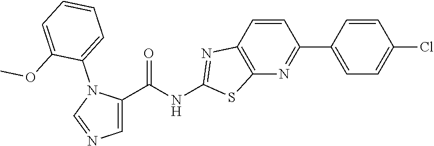 [SMILES: COc1ccccc1-n1cncc1C(=O)Nc1nc2ccc(-c3ccc(Cl)cc3)nc2s1]

### Step 1: ethyl 1-(2-methoxyphenyl)-1H-imidazole-5-carboxylate

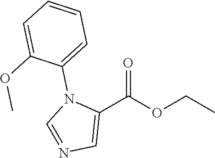 [SMILES: CCOC(=O)c1cncn1-c1ccccc1OC]

The reaction mixture of 2-methoxyaniline (1.3 g, 10 mmol), ethyl 2-oxoacetate (50% in water, 2.04 g, 10 mmol) and Na₂SO₄ (7.1 g, 50 mmol) in toluene (30 mL) was stirred at 120° C. for 2 h. After cooled to room temperature, the mixture was filtered and the filtrate was evaporated. The residue was dissolved with ethanol, then K₂CO₃ (916 mg, 6.6 mmol) and tosylmethyl isocyanide (975 mg, 5 mmol) were added.

The mixture was stirred at 50° C. for 14 h. The solvent was evaporated and the residue was purified over silica gel by combi-flash, eluting with ethyl acetate in petroleum ether (50%, v/v) to give the title compound (620 mg, 25%). LC-MS (M+H)⁺=247.1.

### Step 2: 1-(2-methoxyphenyl)-1H-imidazole-5-carboxylic acid

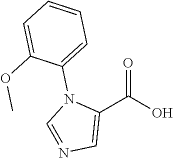 [SMILES: COc1ccccc1-n1cncc1C(=O)O]

The title compound (280 mg, 45%) was prepared in a manner similar to Example 1 step 3 from ethyl 1-(2-methoxyphenyl)-1H-imidazole-5-carboxylate. ¹H NMR (400 MHz, d6-DMSO) δ 12.82 (s, 1H), 8.02 (s, 1H), 7.75 (s, 1H), 7.66 (dd, J=7.8, 1.3 Hz, 1H), 7.61-7.47 (m, 3H). LC-MS (M+H)⁺=219.0.

### Step 3: N-(5-(4-chlorophenyl)thiazolo[5,4-b]pyridin-2-yl)-1-(2-methoxyphenyl)-1H-imidazole-5-carboxamide

The title compound (21 mg, 13%) was prepared in a manner similar to Example 11 step 3 from 5-(4-chlorophenyl)thiazolo[5,4-b]pyridin-2-amine and 1-(2-methoxyphenyl)-1H-imidazole-5-carboxylic acid. 1H NMR (400 MHz, d6-DMSO) δ 13.11 (s, 1H), 8.30-8.10 (m, 5H), 8.04 (s, 1H), 7.57 (d, J=8.0 Hz, 2H), 7.47 (t, J=7.0 Hz, 1H), 7.43 (d, J=6.8 Hz, 1H), 7.21 (d, J=8.0 Hz, 1H) 7.10 (t, J=7.0 Hz, 1H), 3.70 (s, 3H). LC-MS (M+H)⁺=462.1.

### Example 13: N-(5-(4-chlorophenyl)thiazolo[5,4-b]pyridin-2-yl)-6-cyano-2-(3-oxopiperazin-1-yl)nicotinamide

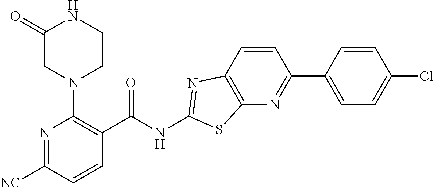 [SMILES: N#Cc1ccc(C(=O)Nc2nc3ccc(-c4ccc(Cl)cc4)nc3s2)c(N2CCNC(=O)C2)n1]

### Step 1: 2-chloro-N-(5-(4-chlorophenyl)thiazolo[5,4-b]pyridin-2-yl)-6-cyanonicotinamide

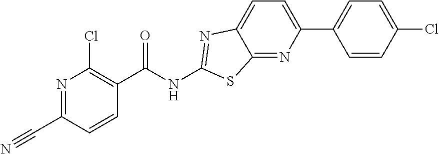 [SMILES: N#Cc1ccc(C(=O)Nc2nc3ccc(-c4ccc(Cl)cc4)nc3s2)c(Cl)n1]

The title compound (274 mg, 79%) was prepared in a manner similar to that in Example 4 step 2 from 2-chloro-6-cyanonicotinic acid and 5-(4-chlorophenyl)thiazolo[5,4-b]pyridin-2-amine. LC-MS (M+H)⁺=426.0.

### Step 2: N-(5-(4-chlorophenyl)thiazolo[5,4-b]pyridin-2-yl)-6-cyano-2-(3-oxopiperazin-1-yl)nicotinamide

To a stirred solution of 2-chloro-N-(5-(4-chlorophenyl)thiazolo[5,4-b]pyridin-2-yl)-6-cyanonicotinamide (215 mg, 0.506 mmol) and piperazin-2-one (102 mg, 1.019 mmol) in dioxane (10 mL) were added N,N-diisopropylethylamine (262 mg, 2.027 mmol) at room temperature. The resulting mixture was stirred for 16 h at 80° C. under nitrogen atmosphere. After consumption of starting material, the resulting mixture was filtered and the filter cake was washed with dichloromethane (2×6 mL). The filtrate was concentrated under reduced pressure and the residue was purified by prep-HPLC under the following conditions: column, XBridge Shield RP18 OBD Column, 30×150 mm, 5 um; mobile phase, acetonitrile in water (with 10 mmol/L NH₄HCO₃ and 0.1% NH₄OH), 20% to 50% gradient in 8 min; detector, UV 254 nm. The title compound (9 mg, 4%) was obtained. 1H NMR (300 MHz, d6-DMSO) δ 13.17 (brs, 1H), 8.30 (d, J=7.8 Hz, 1H), 8.25-8.16 (m, 3H), 8.16-8.07 (m, 2H), 7.64-7.50 (m, 3H), 3.87 (s, 2H), 3.64 (t, J=4.0 Hz, 2H), 3.30-3.20 (m, 2H). LC-MS (M+H)⁺=490.1.

### Example 14: N-(5-(4-chlorophenyl)thiazolo[5,4-b]pyridin-2-yl)-4-(2-methoxyphenyl)-6-methylnicotinamide

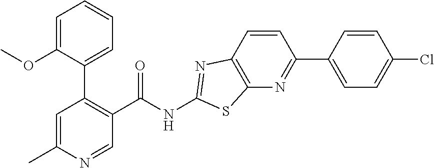 [SMILES: COc1ccccc1-c1cc(C)ncc1C(=O)Nc1nc2ccc(-c3ccc(Cl)cc3)nc2s1]

### Step 1: benzyl 4-chloro-6-methylnicotinate

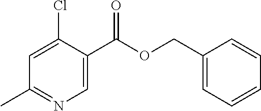 [SMILES: Cc1cc(Cl)c(C(=O)OCc2ccccc2)cn1]

The title compound (4.0 g, 94%) was prepared in a manner similar to that in Example 2 step 1 from 4-chloro-6-methylnicotinic acid and benzyl bromide. LC-MS (M+H)⁺=262.1.

### Step 2: 4-(2-methoxyphenyl)-6-methylnicotinic acid

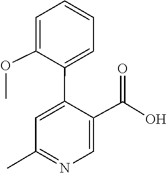 [SMILES: COc1ccccc1-c1cc(C)ncc1C(=O)O]

To a mixture of benzyl 4-chloro-6-methylnicotinate (4.0 g, 15.3 mmol) and (2-methoxyphenyl)boronic acid (2.8 g, 18.4 mmol) in dioxane (50 mL) and water (10 mL) was added K₂CO₃ (6.3 g, 18.4 mmol) and [1,1′-bis(diphenylphosphino)ferrocene]dichloro-palladium(II) (600 mg, 0.8 mmol). The mixture was stirred at 80° C. for 14 h under nitrogen atmosphere. The reaction was cooled to room temperature and diluted with water (30 mL). The mixture was then extracted with ethyl acetate (2×50 mL). The aqueous layer was acidified with 2N HCl solution to pH=2 and then was filtered. The filter cake was dried under vacuum to give title compound (300 mg, 8%). LC-MS (M+H)⁺=244.1.

### Step 3: N-(5-(4-chlorophenyl)thiazolo[5,4-b]pyridin-2-yl)-4-(2-methoxyphenyl)-6-methylnicotinamide

The title compound (15 mg, 15%) was prepared in a manner similar to that in Example 11 step 3 from 4-(2-methoxyphenyl)-6-methylnicotinic acid and 5-(4-chlorophenyl)thiazolo[5,4-b]pyridin-2-amine. ¹H-NMR (400 MHz, d6-DMSO) δ 12.98 (s, 1H), 8.74 (s, 1H), 8.25-8.06 (m, 4H), 7.57 (d, J=7.2 Hz, 2H), 7.45-7.30 (m, 3H), 7.14-7.07 (m, 1H), 6.99 (d, J=8.8 Hz, 1H), 3.51 (s, 3H), 2.59 (s, 3H). LC-MS (M+H)⁺=487.1, 489.0.

### Example 15: 6-cyano-N-(5-(1-methyl-1H-pyrazol-5-yl)thiazolo[5,4-b]pyridin-2-yl)-2-morpholinonicotinamide

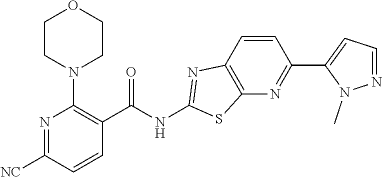 [SMILES: Cn1nccc1-c1ccc2nc(NC(=O)c3ccc(C#N)nc3N3CCOCC3)sc2n1]

### Step 1: 5-(1-methyl-1H-pyrazol-5-yl)thiazolo[5,4-b]pyridin-2-amine

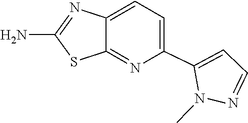 [SMILES: Cn1nccc1-c1ccc2nc(N)sc2n1]

The title compound (110 mg, 23%) was prepared in a manner similar to that in Example 1 step 5 from 5-bromothiazolo[5,4-b]pyridin-2-amine and 1-methyl-5-(4,4,5,5-tetramethyl-1,3,2-dioxaborolan-2-yl)-1H-pyrazole. LC-MS (M+H)⁺=232.0.

### Step 2: 6-cyano-N-(5-(1-methyl-1H-pyrazol-5-yl)thiazolo[5,4-b]pyridin-2-yl)-2-morpholinonicotinamide

The title compound (14 mg, 6%) was prepared in a manner similar to that in Example 4 step 2 from 5-(1-methyl-1H-pyrazol-5-yl)thiazolo[5,4-b]pyridin-2-amine and 6-cyano-2-morpholinonicotinic acid.

¹H-NMR (300 MHz, d6-DMSO) δ 13.23 (brs, 1H), 8.24 (d, J=8.5 Hz, 1H), 8.09 (d, J=7.6 Hz, 1H), 7.91 (d, J=8.5 Hz, 1H), 7.55-7.47 (m, 2H), 6.86 (s, 1H), 4.21-4.15 (m, 3H), 3.68-3.59 (m, 4H), 3.44-3.32 (m, 4H). LC-MS (M+H)⁺=447.1.

### Example 16: N-(5-(4-acetamidophenyl)thiazolo[5,4-b]pyridin-2-yl)-5-(2-methoxyphenyl)pyridazine-4-carboxamide

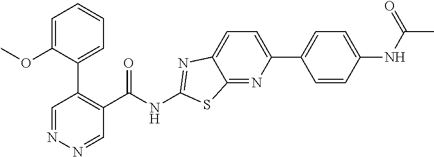 [SMILES: COc1ccccc1-c1cnncc1C(=O)Nc1nc2ccc(-c3ccc(NC(C)=O)cc3)nc2s1]

The title compound (15 mg, 30%) was prepared in a manner similar to that in Example 11 step 3 from 5-(2-methoxyphenyl)pyridazine-4-carboxylic acid and N-(4-(2-aminothiazolo[5,4-b]pyridin-5-yl)phenyl)acetamide. ¹H NMR (400 MHz, d6-DMSO) δ 13.24 (brs, 1H), 10.11 (s, 1H), 9.49 (s, 1H), 9.39 (s, 1H), 8.20-7.96 (m, 4H), 7.72 (d, J=8.7 Hz, 2H), 7.57-7.44 (m, 2H), 7.17-7.11 (m, 1H), 7.07 (d, J=8.3 Hz, 1H), 3.56 (s, 3H), 2.08 (s, 3H). LC-MS (M+H)⁺=497.2.

### Example 17: N-(5-(4-acetamidophenyl)thiazolo[5,4-b]pyridin-2-yl)-4-(5-cyano-2-methoxyphenyl)-6-methylnicotinamide

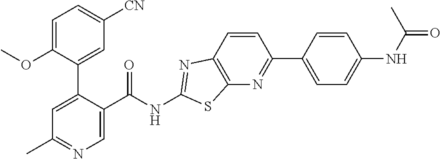 [SMILES: COc1ccc(C#N)cc1-c1cc(C)ncc1C(=O)Nc1nc2ccc(-c3ccc(NC(C)=O)cc3)nc2s1]

The title compound (30 mg, 31%) was prepared in a manner similar to that in Example 11 step 3 from 4-(5-cyano-2-methoxyphenyl)-6-methylnicotinic acid and N-(4-(2-aminothiazolo[5,4-b]pyridin-5-yl)phenyl)acetamide. ¹H NMR (400 MHz, d6-DMSO) δ 12.96 (brs, 1H), 10.11 (s, 1H), 8.80 (s, 1H), 8.14 (d, J=7.8 Hz, 1H), 8.08 (d, J=8.8 Hz, 2H), 8.02 (d, J=8.6 Hz, 1H), 7.95-7.88 (m, 2H), 7.72 (d, J=8.7 Hz, 2H), 7.43 (s, 1H), 7.18 (d, J=8.5 Hz, 1H), 3.60 (s, 3H), 2.60 (s, 3H), 2.08 (s, 3H). LC-MS (M+H)⁺=535.2.

### Example 18: 4-(5-cyano-2-methoxyphenyl)-6-methyl-N-(5-(4-(methylsulfonamido)phenyl)thiazolo[5,4-b]pyridin-2-yl)nicotinamide

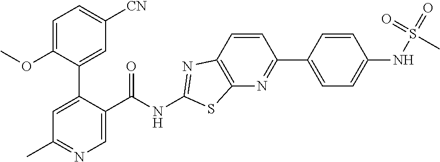

### Step 1: tert-butyl (5-(4-(methylsulfonamido)phenyl)thiazolo[5,4-b]pyridin-2-yl)carbamate

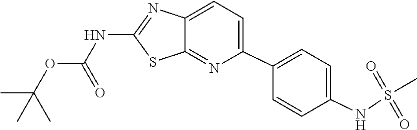 [SMILES: CC(C)(C)OC(=O)Nc1nc2ccc(-c3ccc(NS(C)(=O)=O)cc3)nc2s1]

To a mixture of tert-butyl (5-(4-aminophenyl)thiazolo[5,4-b]pyridin-2-yl)carbamate (50 mg, 0.15 mmol) in dichloromethane (5 mL) were added N-ethyl-N-isopropylpropan-2-amine (100 mg, 0.77 mmol) and methanesulfonic anhydride (50 mg, 0.3 mmol). The mixture was stirred at room temperature for 14 h. The mixture was diluted with water and extracted with dichloromethane (3×20 mL). The combined organic layers were dried over Na₂SO₄, filtered and concentrated under vacuum. The residue was purified over silica gel by combi-flash, eluting with ethyl acetate in petroleum ether (50%, v/v) to give the title compound (50 mg, 79%). LC-MS (M+H)⁺=421.1.

### Step 2: N-(4-(2-aminothiazolo[5,4-b]pyridin-5-Yl)phenyl)methanesulfonamide

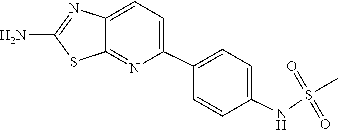 [SMILES: CS(=O)(=O)Nc1ccc(-c2ccc3nc(N)sc3n2)cc1]

The title compound (40 mg, crude) was prepared in a manner similar to that in Example 1 step 6 from tert-butyl (5-(4-(methylsulfonamido)phenyl)thiazolo[5,4-b]pyridin-2-yl)carbamate. LC-MS (M+H)⁺=321.1.

### Step 3: 4-(5-cyano-2-methoxyphenyl)-6-methyl-N-(5-(4-(methylsulfonamido)phenyl)thiazolo[5,4-b]pyridin-2-yl)nicotinamide

The title compound (10 mg, 10%) was prepared in a manner similar to that in Example 11 step 3 from 4-(5-cyano-2-methoxyphenyl)-6-methylnicotinic acid and N-(4-(2-aminothiazolo[5,4-b]pyridin-5-yl)phenyl)methanesulfonamide. ¹H NMR (400 MHz, d6-DMSO) δ 12.94 (brs, 1H), 9.98 (s, 1H), 8.80 (s, 1H), 8.15 (d, J=6.8 Hz, 1H), 8.10 (d, J=8.6 Hz, 2H), 8.02 (d, J=8.5 Hz, 1H), 7.93-7.88 (m, 2H), 7.43 (s, 1H), 7.32 (d, J=8.6 Hz, 2H), 7.18 (d, J=8.4 Hz, 1H), 3.60 (s, 3H), 3.06 (s, 3H), 2.60 (s, 3H). LC-MS (M+H)⁺=571.3.

### Example 19: 4-(5-cyano-2-methoxyphenyl)-N-(5-(4-cyanophenyl)thiazolo[5,4-b]pyridin-2-yl)-6-methylnicotinamide

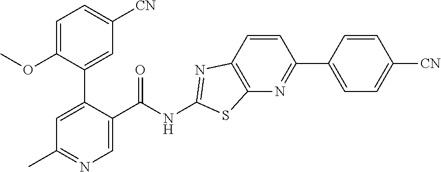 [SMILES: COc1ccc(C#N)cc1-c1cc(C)ncc1C(=O)Nc1nc2ccc(-c3ccc(C#N)cc3)nc2s1]

### Step 1: tert-butyl (5-(4-cyanophenyl)thiazolo[5,4-b]pyridin-2-yl)carbamate

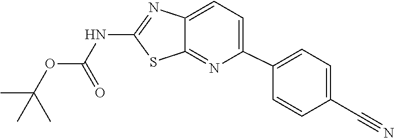 [SMILES: CC(C)(C)OC(=O)Nc1nc2ccc(-c3ccc(C#N)cc3)nc2s1]

The title compound (270 mg, 77%) was prepared in a manner similar to that in Example 1 step 5 from tert-butyl (5-bromothiazolo[5,4-b]pyridin-2-yl)carbamate and (4-cyanophenyl)boronic acid. LC-MS (M+H)⁺=353.1.

### Step 2: 4-(2-aminothiazolo[5,4-b]pyridin-5-yl)benzonitrile

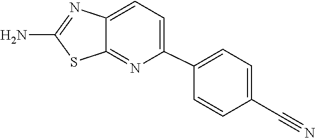 [SMILES: N#Cc1ccc(-c2ccc3nc(N)sc3n2)cc1]

The title compound (150 mg, 77%) was prepared in a manner similar to that in Example 1 step 6 from tert-butyl (5-(4-cyanophenyl)thiazolo[5,4-b]pyridin-2-yl)carbamate. LC-MS (M+H)⁺=253.1.

### Step 3: 4-(5-cyano-2-methoxyphenyl)-N-(5-(4-cyanophenyl)thiazolo[5,4-b]pyridin-2-yl)-6-methylnicotinamide

The title compound (55 mg, 54%) was prepared in a manner similar to that in Example 11 step 3 from 4-(5-cyano-2-methoxyphenyl)-6-methylnicotinic acid and 4-(2-aminothiazolo[5,4-b]pyridin-5-yl)benzonitrile. ¹H-NMR (400 MHz, d6-DMSO) δ 13.08 (s, 1H), 8.82 (s, 1H), 8.34 (d, J=8.4 Hz, 2H), 8.22 (s, 2H), 7.97 (d, J=8.4 Hz, 2H), 7.94-7.85 (m, 2H), 7.43 (s, 1H), 7.18 (d, J=8.4 Hz, 1H), 3.60 (s, 3H), 2.60 (s, 3H). LC-MS (M+H)⁺=503.1.

### Example 20: 4-(5-cyano-2-methoxyphenyl)-N-(5-(4-cyclopropylphenyl)thiazolo[5,4-b]pyridin-2-yl)-6-methylnicotinamide

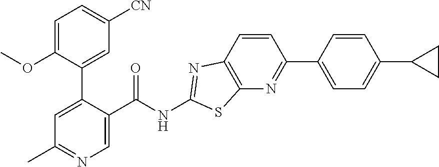 [SMILES: COc1ccc(C#N)cc1-c1cc(C)ncc1C(=O)Nc1nc2ccc(-c3ccc(C4CC4)cc3)nc2s1]

### Step 1: tert-butyl (5-(4-cyclopropylphenyl thiazolo[5,4-b]pyridin-2-yl)carbamate

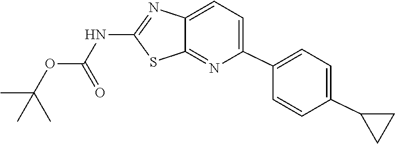 [SMILES: CC(C)(C)OC(=O)Nc1nc2ccc(-c3ccc(C4CC4)cc3)nc2s1]

The title compound (140 mg, 52%) was prepared in a manner similar to that in Example 1 step 5 from tert-butyl (5-bromothiazolo[5,4-b]pyridin-2-yl)carbamate and (4-cyclopropylphenyl)boronic acid. LC-MS (M+H)⁺=368.0.

### Step 2: 5-(4-cyclopropylphenyl)thiazolo[5,4-b]pyridin-2-amine

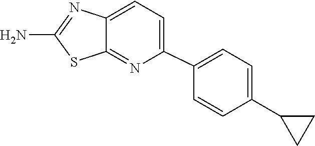 [SMILES: Nc1nc2ccc(-c3ccc(C4CC4)cc3)nc2s1]

The title compound (100 mg, 72%) was prepared in a manner similar to that in Example 1 step 6 from tert-butyl (5-(4-cyclopropylphenyl)thiazolo[5,4-b]pyridin-2-yl)carbamate. LC-MS (M+H)⁺=268.1.

### Step 3: 4-(5-cyano-2-methoxyphenyl)-N-(5-(4-cyclopropylphenyl)thiazolo[5,4-b]pyridin-2-yl)-6-methylnicotinamide

The title compound (15 mg, 16%) was prepared in a manner similar to that in Example 11 step 3 from 5-(4-cyclopropylphenyl)thiazolo[5,4-b]pyridin-2-amine and 4-(5-cyano-2-methoxyphenyl)-6-methylnicotinic acid. ¹H-NMR (400 MHz, d6-DMSO) δ 12.96 (s, 1H), 8.81 (s, 1H), 8.14 (d, J=9.1 Hz, 1H), 8.08-7.97 (m, 3H), 7.96-7.84 (m, 2H), 7.43 (s, 1H), 7.19 (t, J=8.4 Hz, 3H), 3.60 (s, 3H), 2.60 (s, 3H), 2.07-1.85 (m, 1H), 1.01 (dd, J=8.3, 2.0 Hz, 2H), 0.81-0.66 (m, 2H). LC-MS (M+H)⁺=518.2.

### Example 21: N-(5-(4-carbamoylphenyl)thiazolo[5,4-b]pyridin-2-yl)-4-(5-cyano-2-methoxyphenyl)-6-methylnicotinamide

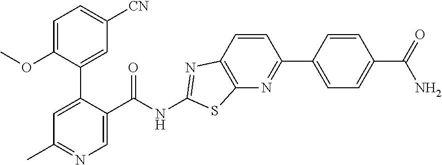 [SMILES: COc1ccc(C#N)cc1-c1cc(C)ncc1C(=O)Nc1nc2ccc(-c3ccc(C(N)=O)cc3)nc2s1]

### Step 1: N-(5-bromothiazolo[5,4-b]pyridin-2-yl)-4-(5-cyano-2-methoxyphenyl)-6-methylnicotinamide

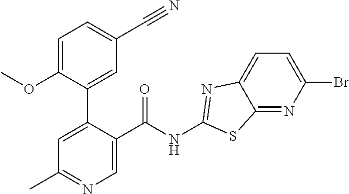 [SMILES: COc1ccc(C#N)cc1-c1cc(C)ncc1C(=O)Nc1nc2ccc(Br)nc2s1]

The title compound (120 mg, 48%) was prepared in a manner similar to that in Example 11 step 3 from 5-bromothiazolo[5,4-b]pyridin-2-amine and 4-(5-cyano-2-methoxyphenyl)-6-methylnicotinic acid. LC-MS (M+H)⁺=480.0, 482.0.

### Step 2: N-(5-(4-carbamoylphenyl)thiazolo[5,4-b]pyridin-2-yl)-4-(5-cyano-2-methoxyphenyl)-6-methylnicotinamide

The title compound (2 mg, 2%) was prepared in a manner similar to that in Example 1 step 5 from N-(5-bromothiazolo[5,4-b]pyridin-2-yl)-4-(5-cyano-2-methoxyphenyl)-6-methylnicotinamide and (4-carbamoylphenyl)boronic acid. ¹H-NMR (400 MHz, d6-DMSO) δ 8.87 (s, 1H), 8.32-8.15 (m, 2H), 8.06 (s, 2H), 7.99 (d, J=8.4 Hz, 2H), 7.89 (d, J=8.5 Hz, 1H), 7.83 (s, 1H), 7.45-7.25 (m, 2H), 7.17 (d, J=8.7 Hz, 1H), 3.60 (s, 3H), 2.56 (s, 3H). LC-MS (M+H)⁺=521.2.

### Example 22: N-(5-(4-acetamidophenyl)thiazolo[5,4-b]pyridin-2-yl)-3-(5-cyano-2-methoxyphenyl)isonicotinamide

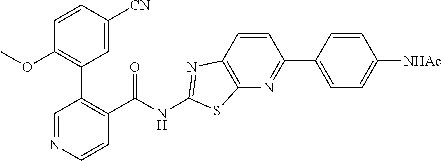 [SMILES: COc1ccc(C#N)cc1-c1cnccc1C(=O)Nc1nc2ccc(-c3ccc(NC(C)=O)cc3)nc2s1]

The title compound (17 mg, 18%) was prepared in a manner similar to that in Example 11 step 3 from 3-(5-cyano-2-methoxyphenyl)isonicotinic acid and N-(4-(2-aminothiazolo[5,4-b]pyridin-5-yl)phenyl)acetamide. ¹H-NMR (400 MHz, d6-DMSO) δ 13.07 (s, 1H), 10.12 (s, 1H), 8.82 (d, J=4.9 Hz, 1H), 8.71 (s, 1H), 8.24-8.13 (m, 1H), 8.05 (dd, J=20.8, 8.7 Hz, 3H), 7.97-7.86 (m, 2H), 7.74 (dd, J=19.5, 6.8 Hz, 3H), 7.18 (d, J=8.7 Hz, 1H), 3.60 (s, 3H), 2.08 (s, 3H). LC-MS (M+H)⁺=521.2.

### Example 23: N-(5-(4-acetamidophenyl)thiazolo[5,4-b]pyridin-2-yl)-5-(5-cyano-2-methoxyphenyl)pyridazine-4-carboxamide

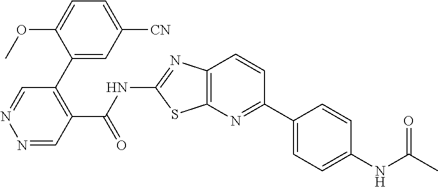 [SMILES: COc1ccc(C#N)cc1-c1cnncc1C(=O)Nc1nc2ccc(-c3ccc(NC(C)=O)cc3)nc2s1]

### Step 1: 5-(5-cyano-2-methoxyphenyl)pyridazine-4-carboxylic acid

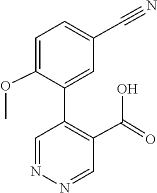 [SMILES: COc1ccc(C#N)cc1-c1cnncc1C(=O)O]

To a mixture of 3-bromo-4-methoxybenzonitrile (200 mg, 1 mmol) and pyridazine-4-carboxylic acid (100 mg, 1 mmol) in N,N-dimethylformamide (5 mL) was added Cs₂CO₃ (900 mg, 3 mmol), Pd(OAc)₂ (20 mg, 0.1 mmol) and di(1-adamantyl)-n-butylphosphine hydriodide (50 mg, 0.1 mmol). The mixture was stirred at 150° C. for 15 h under nitrogen atmosphere. The mixture was cooled and diluted with water (20 mL). The resulting solution was washed with ethyl acetate (2×20 mL). The aqueous layer was acidified with 2N HCl to pH=2, then extracted with ethyl acetate (3×30 mL). The combined organic layers were washed with brine, dried over Na₂SO₄, filtered and concentrated under vacuum. The residue was purified over silica gel by combi-flash, eluting with methanol in dichloromethane (7%, v/v) to give the title compound (20 mg, 8%). LC-MS (M+H)⁺=256.1.

### Step 2: N-(5-(4-acetamidophenyl)thiazolo[5,4-b]pyridin-2-yl)-5-(5-cyano-2-methoxyphenyl)pyridazine-4-carboxamide

The title compound (2 mg, 5%) was prepared in a manner similar to that in Example 11 step 3 from 5-(5-cyano-2-methoxyphenyl)pyridazine-4-carboxylic acid and N-(4-(2-aminothiazolo[5,4-b]pyridin-5-yl)phenyl)acetamide. ¹H NMR (400 MHz, d6-DMSO) δ 12.99 (brs, 1H), 10.06 (s, 1H), 9.57 (s, 1H), 9.27 (brs, 1H), 8.16 (s, 1H), 8.03 (d, J=8.6 Hz, 2H), 7.96-7.80 (m, 3H), 7.68 (d, J=8.8 Hz, 2H), 7.23 (d, J=9.0 Hz, 1H), 3.64 (s, 3H), 2.07 (s, 3H). LCMS (M+H)⁺=522.3.

### Example 24: N-(5-(4-acetamidophenyl)thiazolo[5,4-b]pyridin-2-yl)-4-(2-methoxy-5-(trifluoromethyl)phenyl)-6-methylnicotinamide

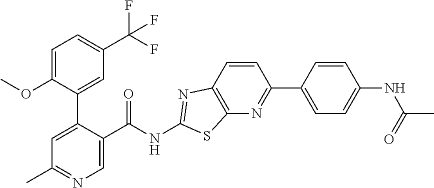 [SMILES: COc1ccc(C(F)(F)F)cc1-c1cc(C)ncc1C(=O)Nc1nc2ccc(-c3ccc(NC(C)=O)cc3)nc2s1]

### Step 1: methyl 4-(2-methoxy-5-(trifluoromethyl)phenyl)-6-methylnicotinate

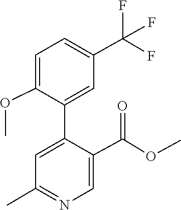 [SMILES: COC(=O)c1cnc(C)cc1-c1cc(C(F)(F)F)ccc1OC]

The title compound (0.6 g, 34%) was prepared in a manner similar to that in Example 2 step 2 from methyl 4-chloro-6-methylnicotinate and (2-methoxy-5-(trifluoromethyl)phenyl)boronic acid. LC-MS (M+H)⁺=326.1.

### Step 2: 4-(2-methoxy-5-(trifluoromethyl)phenyl)-6-methylnicotinic acid

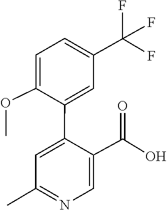 [SMILES: COc1ccc(C(F)(F)F)cc1-c1cc(C)ncc1C(=O)O]

The title compound (0.45 g, 78%) was prepared in a manner similar to that in Example 1 step 3 from methyl 4-(2-methoxy-5-(trifluoromethyl)phenyl)-6-methylnicotinate. LC-MS (M+H)⁺=312.1.

### Step 3: N-(5-(4-acetamidophenyl)thiazolo[5,4-b]pyridin-2-yl)-4-(2-methoxy-5-(trifluoromethyl)phenyl)-6-methylnicotinamide

The title compound (12 mg, 12%) was prepared in a manner similar to that in Example 25 step 3 from 4-(2-methoxy-5-(trifluoromethyl)phenyl)-6-methylnicotinic acid and N-(4-(2-aminothiazolo[5,4-b]pyridin-5-yl)phenyl)acetamide. ¹H-NMR (400 MHz, d6-DMSO) δ 12.97 (s, 1H), 10.11 (s, 1H), 8.80 (s, 1H), 8.15 (d, J=8.4 Hz, 1H), 8.08 (d, J=8.7 Hz, 2H), 8.02 (d, J=8.6 Hz, 1H), 7.79 (d, J=8.8 Hz, 1H), 7.74-7.71 (m, 3H), 7.43 (s, 1H), 7.19 (d, J=8.7 Hz, 1H), 3.60 (s, 3H), 2.61 (s, 3H), 2.08 (s, 3H). LC-MS (M+H)⁺=578.2.

### Example 25: N-(5-(4-acetamidophenyl)thiazolo[5,4-b]pyridin-2-yl)-4-(5-fluoro-2-methoxyphenyl)-6-methylnicotinamide

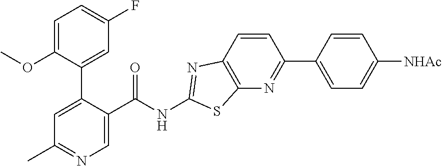 [SMILES: COc1ccc(F)cc1-c1cc(C)ncc1C(=O)Nc1nc2ccc(-c3ccc(NC(C)=O)cc3)nc2s1]

### Step 1: methyl 4-(5-fluoro-2-methoxyphenyl)-6-methylnicotinate

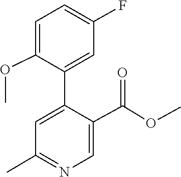 [SMILES: COC(=O)c1cnc(C)cc1-c1cc(F)ccc1OC]

The title compound (550 mg, 100%) was prepared in a manner similar to that in Example 2 step 2 from methyl 4-chloro-6-methylnicotinate and (5-fluoro-2-methoxyphenyl)boronic acid. ¹H-NMR (400 MHz, d6-DMSO) δ 8.77 (s, 1H), 7.32-7.25 (m, 1H), 7.21 (dt, J=9.2, 3.1 Hz, 2H), 7.04 (dd, J=8.9, 4.5 Hz, 1H), 3.64 (s, 3H), 2.55 (s, 3H). LC-MS (M+H)⁺=276.1.

### Step 2: 4-(5-fluoro-2-methoxyphenyl)-6-methylnicotinic acid

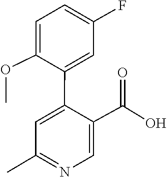 [SMILES: COc1ccc(F)cc1-c1cc(C)ncc1C(=O)O]

The title compound (320 mg, 61%) was prepared in a manner similar to that in Example 1 step 3 from methyl 4-(5-fluoro-2-methoxyphenyl)-6-methylnicotinate. LC-MS (M+H)⁺=262.2.

### Step 3: N-(5-(4-acetamidophenyl)thiazolo[5,4-b]pyridin-2-yl)-4-(5-fluoro-2-methoxyphenyl)-6-methylnicotinamide

The reaction mixture of 4-(5-fluoro-2-methoxyphenyl)-6-methylnicotinic acid (55 mg, 0.21 mmol) and N-(4-(2-aminothiazolo[5,4-b]pyridin-5-yl)phenyl)acetamide (50 mg, 0.18 mmol), EDCI (172 mg, 0.9 mmol), HOBt (122 mg, 0.9 mmol) and pyridine (5 mL) was stirred at 80° C. for 14 h. The solvent was evaporated and the residue was treated with water. The precipitate was filtered and dried. The crude product was purified by prep-HPLC (Elution: acetonitrile (0.1% formic acid)/water (0.1% formic acid): 50%-80%, v/v) to give the title compound (41 mg, 43%). ¹H-NMR (400 MHz, d6-DMSO) δ 12.91 (s, 1H), 10.11 (s, 1H), 8.76 (s, 1H), 8.26-7.93 (m, 4H), 7.72 (d, J=8.7 Hz, 2H), 7.38 (s, 1H), 7.33-7.20 (m, 2H), 6.99 (dd, J=9.1, 4.4 Hz, 1H), 3.50 (s, 3H), 2.59 (s, 3H), 2.08 (s, 3H). LC-MS (M+H)⁺=528.2.

### Example 26: N-(5-(4-acetamidophenyl)thiazolo[5,4-b]pyridin-2-yl)-4-(5-carbamoyl-2-methoxyphenyl)-6-methylnicotinamide

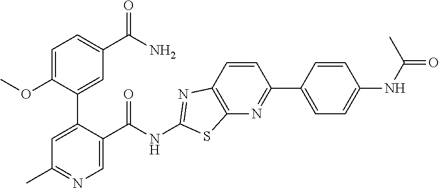 [SMILES: COc1ccc(C(N)=O)cc1-c1cc(C)ncc1C(=O)Nc1nc2ccc(-c3ccc(NC(C)=O)cc3)nc2s1]

### Step 1: 4-(5-carbamoyl-2-methoxyphenyl)-6-methylnicotinic acid

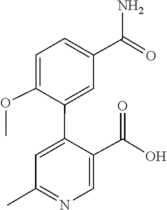 [SMILES: COc1ccc(C(N)=O)cc1-c1cc(C)ncc1C(=O)O]

4-(5-cyano-2-methoxyphenyl)-6-methylnicotinic acid (150 mg, 0.6 mmol) was added to H₂SO₄ (80%, 1 mL). The reaction mixture was stirred at 40° C. for 15 h before cooled to room temperature and diluted with water (10 mL). The mixture was filtered and the filter cake was dried under vacuum to give crude title compound (150 mg). LC-MS (M+H)⁺=287.1.

### Step 2: N-(5-(4-acetamidophenyl)thiazolo[5,4-b]pyridin-2-yl)-4-(5-carbamoyl-2-methoxyphenyl)-6-methylnicotinamide

The title compound (30 mg, 31%) was prepared in a manner similar to that in Example 25 step 3 from 4-(5-carbamoyl-2-methoxyphenyl)-6-methylnicotinic acid and N-(4-(2-aminothiazolo[5,4-b]pyridin-5-yl)phenyl)acetamide. ¹H NMR (400 MHz, d6-DMSO) δ 12.93 (brs, 1H), 10.11 (s, 1H), 8.77 (s, 1H), 8.12 (d, J=8.6 Hz, 1H), 8.07 (d, J=8.7 Hz, 2H), 8.04-7.91 (m, 4H), 7.71 (d, J=8.7 Hz, 2H), 7.40 (s, 1H), 7.31 (s, 1H), 7.04 (d, J=8.4 Hz, 1H), 3.56 (s, 3H), 2.61 (s, 3H), 2.08 (s, 3H). LC-MS (M+H)⁺=553.3.

### Example 27: N-(5-(4-acetamidophenyl)thiazolo[5,4-b]pyridin-2-yl)-6-cyano-4-(2-methoxyphenyl)nicotinamide

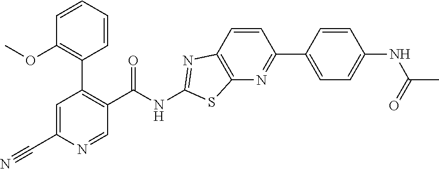 [SMILES: COc1ccccc1-c1cc(C#N)ncc1C(=O)Nc1nc2ccc(-c3ccc(NC(C)=O)cc3)nc2s1]

### Step 1: benzyl 6-bromo-4-chloronicotinate

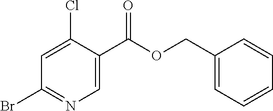 [SMILES: O=C(OCc1ccccc1)c1cnc(Br)cc1Cl]

The title compound (4.023 g, 97%) was prepared in a manner similar to that in Example 2 step 1 from 6-bromo-4-chloronicotinic acid and benzyl bromide. LC-MS (M+H)⁺=326.0, 327.9.

### Step 2: benzyl 4-chloro-6-cyanonicotinate

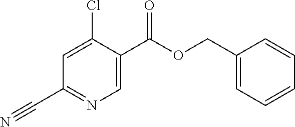 [SMILES: N#Cc1cc(Cl)c(C(=O)OCc2ccccc2)cn1]

The title compound (4.023 g, 97%) was prepared in a manner similar to that in Example 1 Step 2 from benzyl 6-bromo-4-chloronicotinate. LC-MS (M+H)⁺=273.1.

### Step 3: benzyl 6-cyano-4-(2-methoxyphenyl)nicotinate

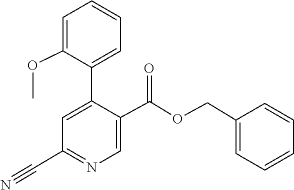 [SMILES: COc1ccccc1-c1cc(C#N)ncc1C(=O)OCc1ccccc1]

The title compound (456 mg, 90%) was prepared in a manner similar to that in Example 2 Step 2 from benzyl 4-chloro-6-cyanonicotinate and (2-methoxyphenyl)boronic acid. LC-MS (M+H)⁺=345.1.

### Step 4: 6-cyano-4-(2-methoxyphenyl)nicotinic acid

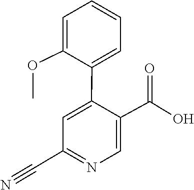 [SMILES: COc1ccccc1-c1cc(C#N)ncc1C(=O)O]

The title compound (98 mg, 29%) was prepared in a manner similar to that in Example 2 Step 3 from benzyl 6-cyano-4-(2-methoxyphenyl)nicotinate. LC-MS (M+H)⁺=255.0.

### Step 5: N-(5-(4-acetamidophenyl)thiazolo[5,4-b]pyridin-2-yl)-6-cyano-4-(2-methoxyphenyl)nicotinamide

The title compound (34 mg, 47%) was prepared in a manner similar to that in Example 25 step 3 from 6-cyano-4-(2-methoxyphenyl)nicotinic acid and N-(4-(2-aminothiazolo[5,4-b]pyridin-5-yl)phenyl)acetamide. ¹H-NMR (400 MHz, d6-DMSO) δ:13.20 (s, 1H), 10.11 (s, 1H), 9.04 (s, 1H), 8.21-8.13 (m, 2H), 8.08 (d, J=8.7 Hz, 2H), 8.03 (d, J=8.6 Hz, 1H), 7.72 (d, J=8.7 Hz, 2H), 7.47 (dd, J=15.5, 7.5 Hz, 2H), 7.13 (t, J=7.4 Hz, 1H), 7.03 (d, J=8.2 Hz, 1H), 3.54 (s, 3H), 2.08 (s, 3H). LC-MS (M+H)⁺=521.2.

### Example 28: N-(5-(4-acetamidophenyl)thiazolo[5,4-b]pyridin-2-yl)-4-(2-methoxy-5-(methoxymethyl)phenyl)-6-methylnicotinamide

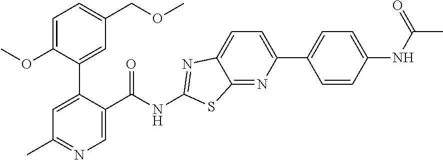 [SMILES: COCc1ccc(OC)c(-c2cc(C)ncc2C(=O)Nc2nc3ccc(-c4ccc(NC(C)=O)cc4)nc3s2)c1]

### Step 1: 2-bromo-1-methoxy-4-(methoxymethylbenzene

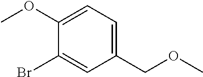

To a solution of (3-bromo-4-methoxyphenyl)methanol (2.17 g, 10 mmol) in anhydrous THF (30 mL) was added NaH (0.48 g, 60%, 12 mmol) slowly at 0° C. followed by addition of iodomethane (1.7 g, 12 mmol). The resulting mixture was stirred at room temperature for 1 h before quenched with water (20 mL). The mixture was extracted with ethyl acetate (2×50 mL). The combined organic layers were dried over Na₂SO₄, filtered and evaporated in vacuo. The residue was purified over silica gel by combi-flash, eluting with ethyl acetate in petroleum ether (10%, v/v) to give the title compound (1.8 g, 78%). ¹H-NMR (400 MHz, d6-DMSO) δ 7.51 (d, J=1.8 Hz, 1H), 7.29 (dd, J=8.4, 1.9 Hz, 1H), 7.08 (d, J=8.4 Hz, 1H), 4.33 (s, 2H), 3.84 (s, 3H), 3.25 (s, 3H). LC-MS (M+H)⁺=230.9, 232.9.

### Step 2: 2-(2-methoxy-5-(methoxymethyl)phenyl)-4,4,5,5-tetramethyl-1,3,2-dioxaborolane

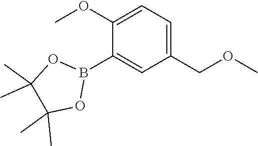

To a mixture of 2-bromo-1-methoxy-4-(methoxymethyl)benzene (1.8 g, 7.8 mmol) and 4,4,4′,4′,5,5,5′,5′-octamethyl-2,2′-bi(1,3,2-dioxaborolane (2.37 g, 9.35 mmol) in dioxane (50 mL) were added [1,1′-bis(diphenylphosphino)ferrocene]dichloropalladium(II) (570 mg, 0.78 mmol) and KOAc (1.53 g, 15.6 mmol). The reaction mixture was stirred at 100° C. for 15 h under nitrogen before cooled to room temperature and diluted with ethyl acetate (50 mL). The organic layer was separated from aqueous layer, washed with brine (50 mL), dried over sodium sulfate, filtered and concentrated under reduced pressure. The residue was purified over silica gel by combi-flash, eluting with ethyl acetate in petroleum ether (16%, v/v) to give the title compound (1.6 g, 74%). LC-MS (M+H)⁺=279.0.

### Step 3: methyl 4-(2-methoxy-5-(methoxymethyl)phenyl)-6-methylnicotinate

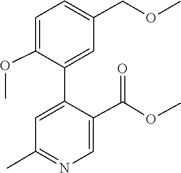 [SMILES: COCc1ccc(OC)c(-c2cc(C)ncc2C(=O)OC)c1]

The title compound (1.2 g, 77%) was prepared in a manner similar to that in Example 2 step 2 from methyl 4-chloro-6-methylnicotinate and 2-(2-methoxy-5-(methoxymethyl)phenyl)-4,4,5,5-tetramethyl-1,3,2-dioxaborolane. LC-MS (M+H)⁺=302.1.

### Step 4: 4-(2-methoxy-5-(methoxymethyl)phenyl)-6-methylnicotinic acid

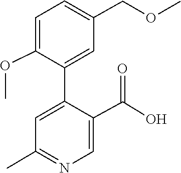 [SMILES: COCc1ccc(OC)c(-c2cc(C)ncc2C(=O)O)c1]

The title compound (0.92 g, 80%) was prepared in a manner similar to that in Example 1 step 3 from methyl 4-(2-methoxy-5-(methoxymethyl)phenyl)-6-methylnicotinate. LC-MS (M+H)⁺=288.1.

### Step 5: N-(5-(4-acetamidophenyl)thiazolo[5,4-b]pyridin-2-yl)-4-(2-methoxy-5-(methoxymethyl)phenyl)-6-methylnicotinamide

The title compound (6 mg, 6%) was prepared in a manner similar to Example 25 step 3 from 4-(2-methoxy-5-(methoxymethyl)phenyl)-6-methylnicotinic acid and N-(4-(2-aminothiazolo[5,4-b]pyridin-5-yl)phenyl)acetamide. ¹H-NMR (400 MHz, d6-DMSO) δ 12.90 (s, 1H), 10.10 (s, 1H), 8.74 (s, 1H), 8.11 (d, J=7.9 Hz, 1H), 8.07 (d, J=8.8 Hz, 2H), 8.00 (d, J=8.5 Hz, 1H), 7.71 (d, J=8.8 Hz, 2H), 7.39-7.29 (m, 3H), 6.96 (d, J=9.0 Hz, 1H), 4.41 (s, 2H), 3.51 (s, 3H), 3.30 (s, 3H), 2.59 (s, 3H), 2.08 (s, 3H). LC-MS (M+H)⁺=554.1.

### Example 29: 4-(5-cyano-2-methoxyphenyl)-N-(5-(4-(1-cyanocyclopropyl)phenyl)thiazolo[5,4-b]pyridin-2-yl)-6-methylnicotinamide

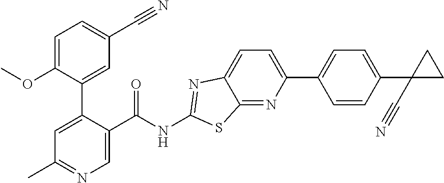 [SMILES: COc1ccc(C#N)cc1-c1cc(C)ncc1C(=O)Nc1nc2ccc(-c3ccc(C4(C#N)CC4)cc3)nc2s1]

### Step 1: tert-butyl (5-(4-(1-cyanocyclopropyl)phenyl)thiazolo[5,4-b]pyridin-2-yl)carbamate

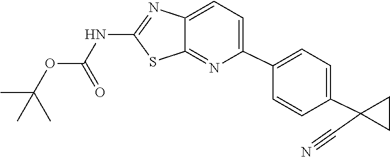 [SMILES: CC(C)(C)OC(=O)Nc1nc2ccc(-c3ccc(C4(C#N)CC4)cc3)nc2s1]

The title compound (crude) was prepared in a manner similar to Example 1 step 5 from tert-butyl (5-bromothiazolo[5,4-b]pyridin-2-yl)carbamate and (4-(1-cyanocyclopropyl)phenyl)boronic acid. LC-MS (M+H)⁺=393.1.

### Step 2: 1-(4-(2-aminothiazolo[5,4-b]pyridin-5-yl)phenyl)cyclopropane-1-carbonitrile

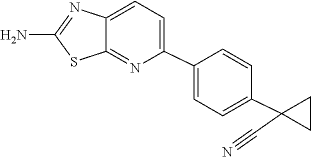 [SMILES: N#CC1(c2ccc(-c3ccc4nc(N)sc4n3)cc2)CC1]

The title compound (220 mg, 75% for 2 steps) was prepared in a manner similar to Example 1 step 6 from tert-butyl (5-(4-(1-cyanocyclopropyl)phenyl)thiazolo[5,4-b]pyridin-2-yl)carbamate. LC-MS (M+H)⁺=293.1.

### Step 3: 4-(5-cyano-2-methoxyphenyl)-N-(5-(4-(1-cyanocyclopropyl)phenyl)thiazolo[5,4-b]pyridin-2-yl)-6-methylnicotinamide

The title compound (15 mg, 14%) was prepared in a manner similar to that in Example 11 step 3 from 4-(5-cyano-2-methoxyphenyl)-6-methylnicotinic acid and 1-(4-(2-aminothiazolo[5,4-b]pyridin-5-yl)phenyl)cyclopropane-1-carbonitrile. ¹H NMR (400 MHz, d6-DMSO) δ 12.99 (s, 1H), 8.80 (s, 1H), 8.24-8.05 (m, 4H), 7.98-7.76 (m, 2H), 7.46 (d, J=8.8 Hz, 3H), 7.18 (d, J=9.3 Hz, 1H), 3.60 (s, 3H), 2.60 (s, 3H), 1.82 (q, J=4.9 Hz, 2H), 1.59 (q, J=5.1 Hz, 2H). LC-MS (M+H)⁺=543.3.

### Example 30: 4-(5-cyano-2-methoxyphenyl)-N-(5-(4-fluorophenyl)thiazolo[5,4-b]pyridin-2-yl)-6-methylnicotinamide

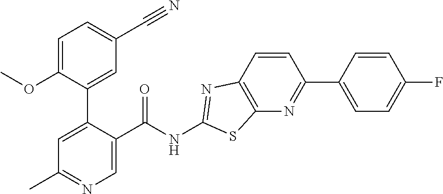 [SMILES: COc1ccc(C#N)cc1-c1cc(C)ncc1C(=O)Nc1nc2ccc(-c3ccc(F)cc3)nc2s1]

### Step 1: tert-butyl (5-(4-fluorophenyl)thiazolo[5,4-b]pyridin-2-yl)carbamate

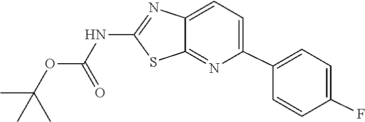 [SMILES: CC(C)(C)OC(=O)Nc1nc2ccc(-c3ccc(F)cc3)nc2s1]

The title compound (crude) was prepared in a manner similar to Example 1 step 5 from tert-butyl (5-bromothiazolo[5,4-b]pyridin-2-yl)carbamate and (4-fluorophenyl)boronic acid. LC-MS (M+H)⁺=346.1.

### Step 2: 5-(4-fluorophenyl)thiazolo[5,4-b]pyridin-2-amine

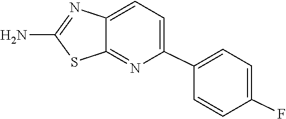 [SMILES: Nc1nc2ccc(-c3ccc(F)cc3)nc2s1]

The title compound (200 mg, 81% for two steps) was prepared in a manner similar to Example 1 step 6 from tert-butyl (5-(4-fluorophenyl)thiazolo[5,4-b]pyridin-2-yl)carbamate. LC-MS (M+H)⁺=246.0.

### Step 3: 4-(5-cyano-2-methoxyphenyl)-N-(5-(4-fluorophenyl)thiazolo[5,4-b]pyridin-2-yl)-6-methylnicotinamide

The title compound (10 mg, 8%) was prepared in a manner similar to Example 11 step 3 from 4-(5-cyano-2-methoxyphenyl)-6-methylnicotinic acid and 5-(4-fluorophenyl)thiazolo[5,4-b]pyridin-2-amine.

¹H NMR (400 MHz, d6-DMSO) δ 12.97 (s, 1H), 8.82 (s, 1H), 8.17 (dt, J=14.7, 7.4 Hz, 3H), 8.05 (d, J=8.6 Hz, 1H), 7.91 (dd, J=11.2, 2.6 Hz, 2H), 7.42 (s, 1H), 7.33 (t, J=8.8 Hz, 2H), 7.18 (d, J=8.6 Hz, 1H), 3.60 (s, 3H), 2.60 (s, 3H). LC-MS (M+H)⁺=496.2.

### Example 31: N-(5-(4-acetamidophenyl)thiazolo[5,4-b]pyridin-2-yl)-3-(2-fluoro-6-methoxyphenyl)isonicotinamide

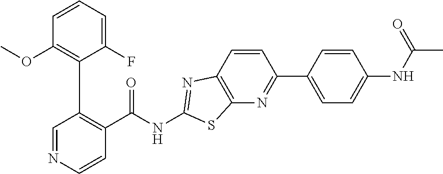 [SMILES: COc1cccc(F)c1-c1cnccc1C(=O)Nc1nc2ccc(-c3ccc(NC(C)=O)cc3)nc2s1]

### Step 1: 2-(2-fluoro-6-methoxyphenyl)-4,4,5,5-tetramethyl-1,3,2-dioxaborolane

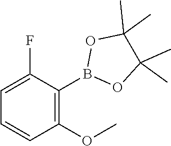 [SMILES: COc1cccc(F)c1B1OC(C)(C)C(C)(C)O1]

To a solution of 2-bromo-1-fluoro-3-methoxybenzene (1 g, 4.9 mmol) in THF (20 mL) under nitrogen was added nBuLi (3 mL, 2.5 M, 7.5 mmol) at −78° C. and stirred for 1 h. Then 2-isopropoxy-4,4,5,5-tetramethyl-1,3,2-dioxaborolane (2.3 g, 12.25 mmol) was added and the reaction mixture was stirred at room temperature for 14 h. The reaction was quenched with water and extracted with ethyl acetate (3×30 mL). The combined organic layers were washed with brine, dried over Na₂SO₄, then filtered and evaporated. The residue was purified over silica gel by combi-flash, eluting with ethyl acetate in petroleum ether (50%, v/v) to give the title compound (740 mg, 60%). LC-MS (M+H)⁺=253.2.

### Step 2: methyl 3-(2-fluoro-6-methoxyphenyl)isonicotinate

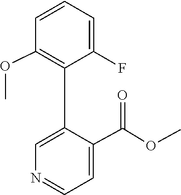 [SMILES: COC(=O)c1ccncc1-c1c(F)cccc1OC]

The title compound (365 mg, 47%) was prepared in a manner similar to Example 2 step 2 from 2-(2-fluoro-6-methoxyphenyl)-4,4,5,5-tetramethyl-1,3,2-dioxaborolane and methyl 3-bromoisonicotinate. LC-MS (M+H)⁺=262.1.

### Step 3: 3-(2-fluoro-6-methoxyphenyl)isonicotinic acid

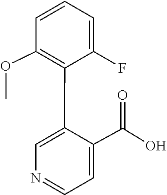 [SMILES: COc1cccc(F)c1-c1cnccc1C(=O)O]

The title compound (190 mg, 77%) was prepared in a manner similar to that in Example 1 step 3 from methyl 3-(2-fluoro-6-methoxyphenyl)isonicotinate. ¹H-NMR (400 MHz, d6-DMSO) δ 13.26 (s, 1H), 8.71 (d, J=5.0 Hz, 1H), 8.55 (s, 1H), 7.77 (d, J=5.0 Hz, 1H), 7.41 (dd, J=15.4, 8.3 Hz, 1H), 7.04-6.79 (m, 2H), 3.70 (s, 3H). LC-MS (M+H)⁺=248.0.

### Step 4: N-(5-(4-acetamidophenyl)thiazolo[5,4-b]pyridin-2-yl)-3-(2-fluoro-6-methoxyphenyl)isonicotinamide

The title compound (32 mg, 24%) was prepared in a manner similar to Example 25 step 3 from 3-(2-fluoro-6-methoxyphenyl)isonicotinic acid and N-(4-(2-aminothiazolo[5,4-b]pyridin-5-yl)phenyl)acetamide. ¹H-NMR (400 MHz, d6-DMSO) δ 13.15 (s, 1H), 10.11 (s, 1H), 8.79 (d, J=5.0 Hz, 1H), 8.66 (d, J=1.5 Hz, 1H), 8.16 (t, J=8.4 Hz, 1H), 8.05 (dd, J=19.5, 8.6 Hz, 3H), 7.81 (d, J=5.0 Hz, 1H), 7.72 (d, J=8.7 Hz, 2H), 7.42 (dd, J=15.3, 8.2 Hz, 1H), 7.10-6.79 (m, 2H), 3.59 (s, 3H), 2.08 (s, 3H). LC-MS (M+H)⁺=514.3.

### Example 32: 4-(5-cyano-2-methoxyphenyl)-6-methyl-N-(5-(4-(N-methylmethylsulfonamido)phenyl)thiazolo[5,4-b]pyridin-2-yl)nicotinamide

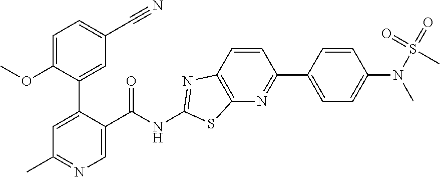

### Step 1: N-(4-(2-aminothiazolo[5,4-b]pyridin-5-yl)phenyl)-N-methylmethanesulfonamide

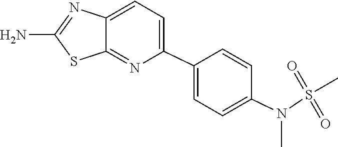 [SMILES: CN(c1ccc(-c2ccc3nc(N)sc3n2)cc1)S(C)(=O)=O]

To a mixture of N-(4-(2-aminothiazolo[5,4-b]pyridin-5-yl)phenyl)methanesulfonamide (80 mg, 0.25 mmol) and K₂CO₃ (70 mg, 0.5 mmol) in N,N-dimethylformamide (2 mL) was added iodomethane (50 mg, 0.35 mmol). The mixture was stirred at room temperature for 3 h before diluted with water. The mixture was extracted with ethyl acetate (3×20 mL). The combined organic layers were washed with brine, dried over Na₂SO₄, filtered and concentrated under vacuum to give title compound (80 mg, crude). LC-MS (M+H)⁺=335.1.

### Step 2: 4-(5-cyano-2-methoxyphenyl)-6-methyl-N-(5-(4-(N-methylmethylsulfonamido)phenyl)thiazolo[5,4-b]pyridin-2-yl)nicotinamide

The title compound (25 mg, 19%) was prepared in a manner similar to that in Example 25 step 3 from 4-(5-cyano-2-methoxyphenyl)-6-methylnicotinic acid and N-(4-(2-aminothiazolo[5,4-b]pyridin-5-yl)phenyl)-N-methylmethanesulfonamide. ¹H NMR (400 MHz, d6-DMSO) δ 12.99 (brs, 1H), 8.81 (s, 1H), 8.22-8.13 (m, 3H), 8.09 (d, J=8.2 Hz, 1H), 7.94-7.88 (m, 2H), 7.54 (d, J=8.6 Hz, 2H), 7.44 (s, 1H), 7.18 (d, J=8.6 Hz, 1H), 3.60 (s, 3H), 3.30 (s, 3H), 2.99 (s, 3H), 2.60 (s, 3H). LC-MS (M+H)⁺=585.3.

### Example 33: 6-chloro-4-(5-cyano-2-methoxyphenyl)-N-(5-(4-cyanophenyl)thiazolo[5,4-b]pyridin-2-yl)nicotinamide

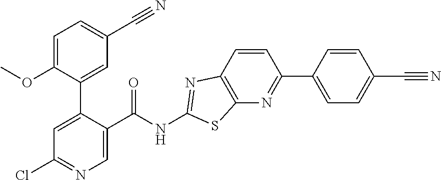 [SMILES: COc1ccc(C#N)cc1-c1cc(Cl)ncc1C(=O)Nc1nc2ccc(-c3ccc(C#N)cc3)nc2s1]

### Step 1: ethyl 6-chloro-4-(5-cyano-2-methoxyphenyl)nicotinate

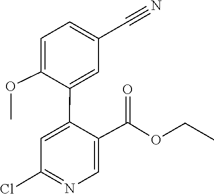 [SMILES: CCOC(=O)c1cnc(Cl)cc1-c1cc(C#N)ccc1OC]

The reaction mixture of ethyl 4,6-dichloronicotinate (5 g, 22.83 mmol), (5-cyano-2-methoxyphenyl)boronic acid (4.04 g, 22.83 mmol), [1,1′-Bis(diphenylphosphino)ferrocene]dichloropalladium(II) (1.67 g, 0.228 mmol) and K₂CO₃ (6.3 g, 45.66 mmol) in 1,4-dioxane/water (100 mL/14 mL) under nitrogen was stirred at 90° C. for 14 h before cooled to room temperature. The mixture was filtered and the filtrate was concentrated. The residue was purified over silica gel by combi-flash, eluting with ethyl acetate in petroleum ether (50%, v/v) to give the title compound (3.2 g, 44.4%). ¹H-NMR (400 MHz, d6-DMSO) δ 8.78 (s, 1H), 7.96 (dd, J=8.6, 1.5 Hz, 1H), 7.89 (s, 1H), 7.67 (s, 1H), 7.27 (d, J=8.7 Hz, 1H), 4.11 (dd, J=14.1, 7.0 Hz, 2H), 3.77 (s, 3H), 1.04 (t, J=7.1 Hz, 3H). LC-MS (M+H)⁺=317.2.

### Step 2: 6-chloro-4-(5-cyano-2-methoxyphenyl)nicotinic acid

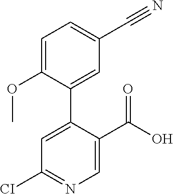

The title compound (100 mg, 74%) was prepared in a manner similar to that in Example 1 of Step 3 from ethyl 6-chloro-4-(5-cyano-2-methoxyphenyl)nicotinate. LC-MS (M+H)⁺=289.2.

### Step 3: 6-chloro-4-(5-cyano-2-methoxyphenyl)-N-(5-(4-cyanophenyl)thiazolo[5,4-b]pyridin-2-yl)nicotinamide

The title compound (15 mg, 14%) was prepared in a manner similar to Example 11 step 3 from 6-chloro-4-(5-cyano-2-methoxyphenyl)nicotinic acid and 4-(2-aminothiazolo[5,4-b]pyridin-5-yl)benzonitrile. ¹H-NMR (400 MHz, d6-DMSO) δ 13.23 (s, 1H), 8.81 (s, 1H), 8.35 (d, J=8.4 Hz, 2H), 8.28-8.22 (m, 2H), 8.01-7.94 (m, 4H), 7.77 (s, 1H), 7.20 (d, J=8.7 Hz, 1H), 3.61 (s, 3H). LC-MS (M+H)⁺=523.2.

### Example 34: 4-(5-cyano-2-methoxyphenyl)-6-(cyanomethyl)-N-(5-(4-cyanophenyl)thiazolo[5,4-b]pyridin-2-yl)nicotinamide

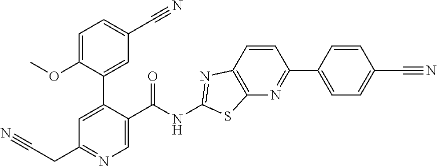 [SMILES: COc1ccc(C#N)cc1-c1cc(CC#N)ncc1C(=O)Nc1nc2ccc(-c3ccc(C#N)cc3)nc2s1]

### Step 1: ethyl 6-(2-(tert-butoxy)-1-cyano-2-oxoethyl)-4-(5-cyano-2-methoxyphenyl)nicotinate

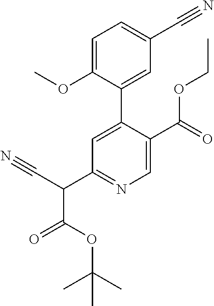 [SMILES: CCOC(=O)c1cnc(C(C#N)C(=O)OC(C)(C)C)cc1-c1cc(C#N)ccc1OC]

The reaction mixture of ethyl 6-chloro-4-(5-cyano-2-methoxyphenyl)nicotinate (0.65 g, 2 mmol), tert-butyl 2-cyanoacetate (0.7 g, 5 mmol), tetrabutylammonium bromide (64 mg, 0.2 mmol) and K₂CO₃ (1.38 g, 10 mmol) in DMSO (10 mL) under nitrogen was stirred at 90° C. for 3 h before cooled to room temperature. The mixture was filtered and the filtrate was concentrated. The residue was purified over silica gel by combi-flash, eluting with ethyl acetate in petroleum ether (0-50%, v/v) to give the title compound (0.88 g, 100%). LC-MS (M+H)⁺=422.5.

### Step 2: ethyl 4-(5-cyano-2-methoxyphenyl)-6-(cyanomethyl)nicotinate

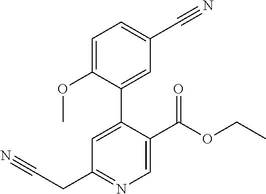 [SMILES: CCOC(=O)c1cnc(CC#N)cc1-c1cc(C#N)ccc1OC]

To a solution of ethyl 6-(2-(tert-butoxy)-1-cyano-2-oxoethyl)-4-(5-cyano-2-methoxyphenyl)nicotinate (0.88 g, 2 mmol) in toluene (10 mL) was added p-toluenesulfonic acid (34 mg, 0.2 mmol). The reaction mixture was stirred at 110° C. for 14 h before cooled to room temperature and concentration. The residue was purified over silica gel by combi-flash, eluting with ethyl acetate in petroleum ether (0-50%, v/v) to give the title compound (0.61 g, 95%). LC-MS (M+H)⁺=322.3.

### Step 3: 4-(5-cyano-2-methoxyphenyl)-6-(cyanomethyl)nicotinic acid

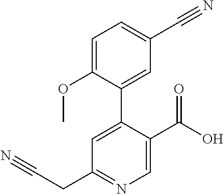 [SMILES: COc1ccc(C#N)cc1-c1cc(CC#N)ncc1C(=O)O]

The title compound (190 mg, 65%) was prepared in a manner similar to that in Example 1 of Step 3 from ethyl 4-(5-cyano-2-methoxyphenyl)-6-(cyanomethyl)nicotinate. LC-MS (M+H)⁺=294.1.

### Step 4: 4-(5-cyano-2-methoxyphenyl)-6-(cyanomethyl)-N-(5-(4-cyanophenyl)thiazolo[5,4-b]pyridin-2-yl)nicotinamide

The title compound (1.5 mg, 1.4%) was prepared in a manner similar to Example 11 step 3 from 4-(5-cyano-2-methoxyphenyl)-6-(cyanomethyl)nicotinic acid and 4-(2-aminothiazolo[5,4-b]pyridin-5-yl)benzonitrile. ¹H-NMR (400 MHz, d6-DMSO) δ 13.18 (s, 1H), 8.94 (s, 1H), 8.35 (d, J=8.5 Hz, 2H), 8.25 (q, J=8.7 Hz, 2H), 7.96 (dd, J=13.5, 8.4 Hz, 4H), 7.59 (s, 1H), 7.21 (d, J=8.4 Hz, 1H), 4.37 (s, 2H), 3.61 (s, 3H). LC-MS (M+H)⁺=528.3.

### Example 35: 4-(5-cyano-2-methoxyphenyl)-N-(5-(4-(ethylsulfonamido)phenyl)thiazolo[5,4-b]pyridin-2-yl)-6-methylnicotinamide

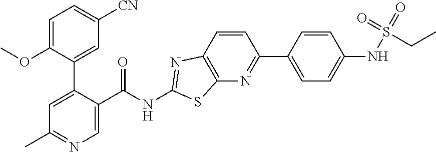 [SMILES: CCS(=O)(=O)Nc1ccc(-c2ccc3nc(NC(=O)c4cnc(C)cc4-c4cc(C#N)ccc4OC)sc3n2)cc1]

### Step 1: tert-butyl (5-(4-(ethylsulfonamido)phenyl)thiazolo[5,4-b]pyridin-2-yl)carbamate

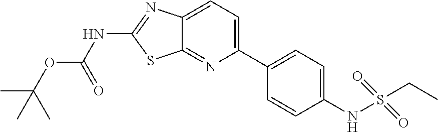 [SMILES: CCS(=O)(=O)Nc1ccc(-c2ccc3nc(NC(=O)OC(C)(C)C)sc3n2)cc1]

To a mixture of tert-butyl (5-(4-aminophenyl)thiazolo[5,4-b]pyridin-2-yl)carbamate (300 mg, 0.88 mmol) in pyridine (10 mL) was added ethanesulfonyl chloride (150 mg, 1.2 mmol) at 0° C. dropwise. The mixture was stirred at 0° C. for 1 h before quenched with water. The mixture was extracted with ethyl acetate (3×30 mL). The combined organic layers were washed with brine, dried over Na₂SO₄, filtered, concentrated under vacuum to give title compound (280 mg, crude). LC-MS (M+H)⁺=435.1.

### Step 2: N-(4-(2-aminothiazolo[5,4-b]pyridin-5-yl)phenyl)ethanesulfonamide

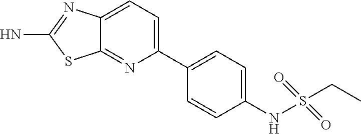 [SMILES: CCS(=O)(=O)Nc1ccc(-c2ccc3nc(N)sc3n2)cc1]

The title compound (180 mg, crude) was prepared in a manner similar to that described in Example 1 step 6 from tert-butyl (5-(4-(ethylsulfonamido)phenyl)thiazolo[5,4-b]pyridin-2-yl)carbamate. LC-MS (M+H)⁺=335.1.

### Step 3: 4-(5-cyano-2-methoxyphenyl)-N-(5-(4-(ethylsulfonamido)phenyl)thiazolo[5,4-b]pyridin-2-yl)-6-methylnicotinamide

The title compound (50 mg, 28%) was prepared in a manner similar to that described in Example 25 step 3 from 4-(5-cyano-2-methoxyphenyl)-6-methylnicotinic acid and N-(4-(2-aminothiazolo[5,4-b]pyridin-5-yl)phenyl)ethanesulfonamide. ¹H NMR (400 MHz, d6-DMSO) δ 12.96 (s, 1H), 10.03 (s, 1H), 8.80 (s, 1H), 8.17 (d, J=8.6 Hz, 1H), 8.09 (d, J=8.7 Hz, 2H), 8.02 (d, J=8.7 Hz, 1H), 7.95-7.87 (m, 2H), 7.44 (s, 1H), 7.34 (d, J=8.7 Hz, 2H), 7.18 (d, J=9.3 Hz, 1H), 3.60 (s, 3H), 3.16 (q, J=7.3 Hz, 2H), 2.60 (s, 3H), 1.21 (t, J=7.3 Hz, 3H). LC-MS (M+H)⁺=585.3.

### Example 36: N-(5-(4-acetamidophenyl)thiazolo[5,4-b]pyridin-2-yl)-6-cyano-4-(5-cyano-2-methoxyphenyl)nicotinamide

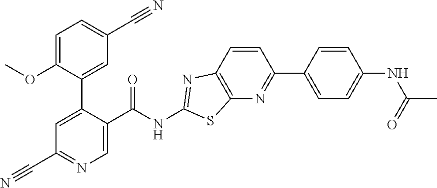 [SMILES: COc1ccc(C#N)cc1-c1cc(C#N)ncc1C(=O)Nc1nc2ccc(-c3ccc(NC(C)=O)cc3)nc2s1]

### Step 1: benzyl 6-cyano-4-(5-cyano-2-methoxyphenyl)nicotinate

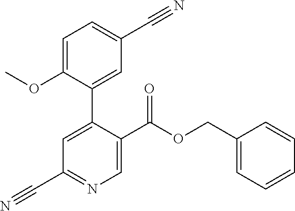 [SMILES: COc1ccc(C#N)cc1-c1cc(C#N)ncc1C(=O)OCc1ccccc1]

The title compound (530 mg, 90%) was prepared in a manner similar to that described in Example 2 step 2 from benzyl 4-chloro-6-cyanonicotinate and (5-cyano-2-methoxyphenyl)boronic acid. LC-MS (M+H)⁺=370.1.

### Step 2: 6-cyano-4-(5-cyano-2-methoxyphenyl)nicotinic acid

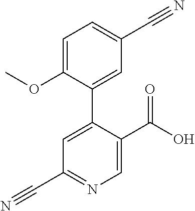 [SMILES: COc1ccc(C#N)cc1-c1cc(C#N)ncc1C(=O)O]

The title compound (143 mg, 36%) was prepared in a manner similar to that described in Example 2 step 3 from benzyl 6-cyano-4-(5-cyano-2-methoxyphenyl)nicotinate. LC-MS (M+H)⁺=280.2.

### Step 3: N-(5-(4-acetamidophenyl)thiazolo[5,4-b]pyridin-2-yl)-6-cyano-4-(5-cyano-2-methoxyphenyl)nicotinamide

The title compound (31 mg, 41%) was prepared in a manner similar to that described in Example 25 step 3 from 6-cyano-4-(5-cyano-2-methoxyphenyl)nicotinic acid and N-(4-(2-aminothiazolo[5,4-b]pyridin-5-yl)phenyl)acetamide. ¹H-NMR (400 MHz, d6-DMSO) δ: 13.26 (s, 1H), 10.11 (s, 1H), 9.11 (s, 1H), 8.29 (s, 1H), 8.21-8.00 (m, 5H), 7.97 (dd, J=8.6, 1.9 Hz, 1H), 7.72 (d, J=8.7 Hz, 2H), 7.22 (d, J=8.7 Hz, 1H), 3.61 (s, 3H), 2.08 (s, 3H). LC-MS (M+H)⁺=546.3.

### Example 37: 4-(5-cyano-2-methoxyphenyl)-N-(5-(4-cyanophenyl)thiazolo[5,4-b]pyridin-2-yl)-6-(trifluoromethyl)nicotinamide

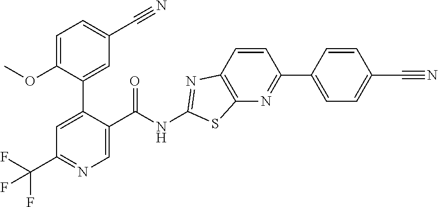 [SMILES: COc1ccc(C#N)cc1-c1cc(C(F)(F)F)ncc1C(=O)Nc1nc2ccc(-c3ccc(C#N)cc3)nc2s1]

### Step 1: methyl 4-(5-cyano-2-methoxyphenyl)-6-(trifluoromethyl)nicotinate

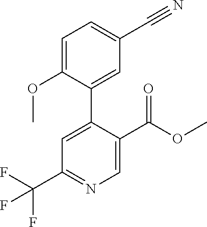 [SMILES: COC(=O)c1cnc(C(F)(F)F)cc1-c1cc(C#N)ccc1OC]

The title compound (0.53 g, 79%) was prepared in a manner similar to that in Example 2 step 2 from methyl 4-chloro-6-(trifluoromethyl)nicotinate and (5-cyano-2-methoxyphenyl)boronic acid. LC-MS (M+H)⁺=337.1.

### Step 2: 4-(5-cyano-2-methoxyphenyl)-6-(trifluoromethyl)nicotinic acid

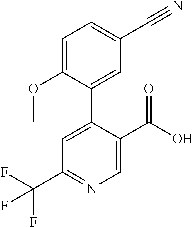 [SMILES: COc1ccc(C#N)cc1-c1cc(C(F)(F)F)ncc1C(=O)O]

The title compound (0.35 g, 66%) was prepared in a manner similar to that in Example 1 step 3 from methyl 4-(5-cyano-2-methoxyphenyl)-6-(trifluoromethyl)nicotinate. LC-MS (M+H)⁺=323.1.

### Step 3: 4-(5-cyano-2-methoxyphenyl)-N-(5-(4-cyanophenyl)thiazolo[5,4-b]pyridin-2-yl)-6-(trifluoromethyl)nicotinamide

The title compound (25 mg, 27%) was prepared in a manner similar to that in Example 25 step 3 from 4-(5-cyano-2-methoxyphenyl)-6-(trifluoromethyl)nicotinic acid and 4-(2-aminothiazolo[5,4-b]pyridin-5-yl)benzonitrile. ¹H-NMR (400 MHz, d6-DMSO) δ 13.34 (s, 1H), 9.14 (s, 1H), 8.35 (d, J=8.4 Hz, 2H), 8.27-8.23 (m, 2H), 8.15-8.08 (m, 2H), 8.02-7.92 (m, 3H), 7.22 (d, J=8.8 Hz, 1H), 3.62 (s, 3H). LC-MS (M+H)⁺=557.2.

### Example 38: 4-(5-cyano-2-methoxyphenyl)-N-(5-(4-cyanophenyl)thiazolo[5,4-b]pyridin-2-yl)nicotinamide

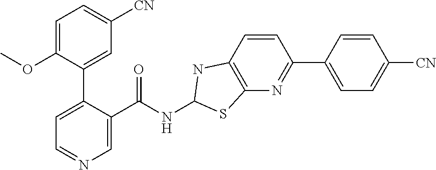 [SMILES: COc1ccc(C#N)cc1-c1ccncc1C(=O)NC1Nc2ccc(-c3ccc(C#N)cc3)nc2S1]

### Step 1: ethyl 4-(5-cyano-2-methoxyphenyl)nicotinate

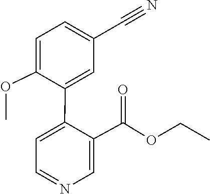 [SMILES: CCOC(=O)c1cnccc1-c1cc(C#N)ccc1OC]

To the solution of ethyl 6-chloro-4-(5-cyano-2-methoxyphenyl)nicotinate (0.15 g, 0.5 mmol) in methanol (5 mL) was added NaHCO₃ (42 mg, 0.5 mmol) and 10 wt. % Pd/C (15 mg), The reaction mixture was stirred under the atmosphere of hydrogen (1 atm) at room temperature for 2 h. The mixture was filtered through a short pad of diatomite. The filtrate was concentrated to give the title compound (0.13 g, crude) that was used for next step without further purification. LC-MS (M+H)⁺=283.2.

### Step 2: 4-(5-cyano-2-methoxyphenyl)nicotinic acid

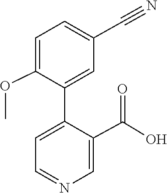 [SMILES: COc1ccc(C#N)cc1-c1ccncc1C(=O)O]

The title compound (90 mg, 71% for 2 steps) was prepared in a manner similar to that in Example 1 of Step 3 from ethyl 4-(5-cyano-2-methoxyphenyl)nicotinate. LC-MS (M+H)⁺=255.3.

### Step 3: 4-(5-cyano-2-methoxyphenyl)-N-(5-(4-cyanophenyl)thiazolo[5,4-b]pyridin-2-yl)nicotinamide

The title compound (20 mg, 20%) was prepared in a manner similar to Example 11 of Step 3 from 4-(5-cyano-2-methoxyphenyl)nicotinic acid and 4-(2-aminothiazolo[5,4-b]pyridin-5-yl)benzonitrile. ¹H-NMR (400 MHz, d6-DMSO) δ 13.12 (s, 1H), 8.96 (s, 1H), 8.82 (d, J=5.4 Hz, 1H), 8.34 (d, J=8.3 Hz, 2H), 8.23-8.15 (m, 2H), 8.01-7.83 (m, 4H), 7.56-7.51 (m, 1H), 7.19 (d, J=8.6 Hz, 1H), 3.61 (s, 3H). LC-MS (M+H)⁺=489.2.

### Example 39: 4-(5-cyano-2-methoxyphenyl)-N-(5-(4-cyanophenyl)thiazolo[5,4-b]pyridin-2-yl)-6-fluoronicotinamide

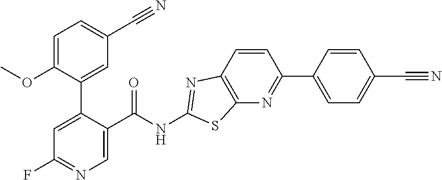 [SMILES: COc1ccc(C#N)cc1-c1cc(F)ncc1C(=O)Nc1nc2ccc(-c3ccc(C#N)cc3)nc2s1]

### Step 1: ethyl 4-(5-cyano-2-methoxyphenyl)-6-fluoronicotinate

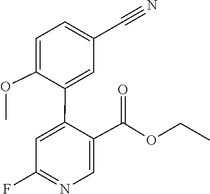 [SMILES: CCOC(=O)c1cnc(F)cc1-c1cc(C#N)ccc1OC]

To a solution of ethyl 6-chloro-4-(5-cyano-2-methoxyphenyl)nicotinate (0.32 g, 1 mmol) in DMSO (5 mL) was added CsF (0.38 g, 2.5 mmol) and the reaction mixture was stirred at 80° C. for 14 h before cooled to room temperature. The mixture was purified over silica gel by combi-flash, eluting with ethyl acetate in petroleum ether (0% to 30%, v/v) to give the title compound (0.25 g, 83%). LC-MS (M+H)⁺=301.1.

### Step 2: 4-(5-cyano-2-methoxyphenyl)-6-fluoronicotinic acid

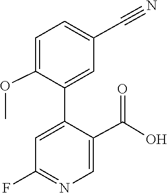 [SMILES: COc1ccc(C#N)cc1-c1cc(F)ncc1C(=O)O]

The title compound (220 mg, 97%) was prepared in a manner similar to that in Example 1 of Step 3 from ethyl 4-(5-cyano-2-methoxyphenyl)-6-fluoronicotinate. LC-MS (M+H)⁺=273.2.

### Step 3: 4-(5-cyano-2-methoxyphenyl)-N-(5-(4-cyanophenyl)thiazolo[5,4-b]pyridin-2-yl)-6-fluoronicotinamide

The title compound (20 mg, 19.8%) was prepared in a manner similar to Example 11 of Step 3 from 4-(5-cyano-2-methoxyphenyl)-6-fluoronicotinic acid and 4-(2-aminothiazolo[5,4-b]pyridin-5-yl)benzonitrile. ¹H-NMR (400 MHz, d6-DMSO) δ 13.20 (s, 1H), 8.68 (s, 1H), 8.35 (d, J=8.5 Hz, 2H), 8.24 (q, J=8.5 Hz, 2H), 7.99-7.95 (m, 4H), 7.46 (s, 1H), 7.21 (d, J=8.7 Hz, 1H), 3.61 (s, 3H). LC-MS (M+H)⁺=507.2.

### Example 40: 4-(5-cyano-2-methoxyphenyl)-6-methyl-N-(5-(4-(trifluoromethyl)phenyl)thiazolo[5,4-b]pyridin-2-yl)nicotinamide

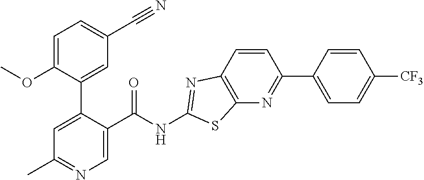 [SMILES: COc1ccc(C#N)cc1-c1cc(C)ncc1C(=O)Nc1nc2ccc(-c3ccc(C(F)(F)F)cc3)nc2s1]

### Step 1: tert-butyl (5-(4-(trifluoromethyl)phenyl)thiazolo[5,4-b]pyridin-2-yl)carbamate

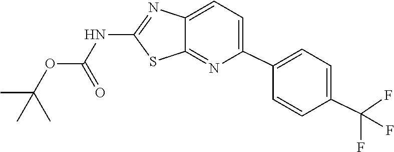 [SMILES: CC(C)(C)OC(=O)Nc1nc2ccc(-c3ccc(C(F)(F)F)cc3)nc2s1]

The title compound (600 mg, 100%) was prepared in a manner similar to that in Example 2 step 2 from tert-butyl (5-bromothiazolo[5,4-b]pyridin-2-yl)carbamate and (4-(trifluoromethyl)phenyl)boronic acid. LC-MS (M+H)⁺=396.1.

### Step 2: 5-(4-(trifluoromethyl)phenyl)thiazolo[5,4-b]pyridin-2-amine

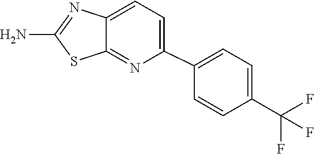 [SMILES: Nc1nc2ccc(-c3ccc(C(F)(F)F)cc3)nc2s1]

The title compound (220 mg, 50%) was prepared in a manner similar to that in Example 1 step 6 from tert-butyl (5-(4-(trifluoromethyl)phenyl)thiazolo[5,4-b]pyridin-2-yl)carbamate. LC-MS (M+H)⁺=296.1.

### Step 3: 4-(5-cyano-2-methoxyphenyl)-6-methyl-N-(5-(4-(trifluoromethyl)phenyl)thiazolo[5,4-b]pyridin-2-yl)nicotinamide

The title compound (50 mg, 31%) was prepared in a manner similar to that in Example 25 step 3 from 4-(5-cyano-2-methoxyphenyl)-6-methylnicotinic acid and 5-(4-(trifluoromethyl)phenyl)thiazolo[5,4-b]pyridin-2-amine. ¹H-NMR (400 MHz, d6-DMSO) δ 13.07 (s, 1H), 8.81 (s, 1H), 8.36 (d, J=8.2 Hz, 2H), 8.25 (d, J=8.5 Hz, 1H), 8.20 (d, J=8.6 Hz, 1H), 7.95-7.90 (m, 2H), 7.87 (d, J=8.4 Hz, 2H), 7.45 (s, 1H), 7.18 (d, J=9.2 Hz, 1H), 3.60 (s, 3H), 2.61 (s, 3H). LC-MS (M+H)⁺=546.2.

### Example 41: 4-(5-cyano-2-methoxyphenyl)-6-methyl-N-(5-(4-propionamidophenyl)thiazolo[5,4-b]pyridin-2-yl)nicotinamide

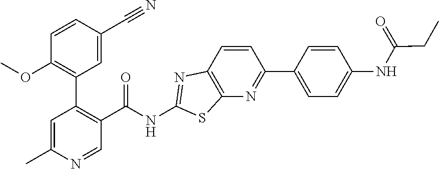 [SMILES: CCC(=O)Nc1ccc(-c2ccc3nc(NC(=O)c4cnc(C)cc4-c4cc(C#N)ccc4OC)sc3n2)cc1]

### Step 1: tert-butyl (5-(4-propionamidophenyl)thiazolo[5,4-b]pyridin-2-yl)carbamate

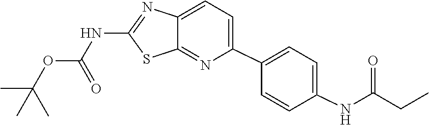 [SMILES: CCC(=O)Nc1ccc(-c2ccc3nc(NC(=O)OC(C)(C)C)sc3n2)cc1]

To a solution of tert-butyl (5-(4-aminophenyl)thiazolo[5,4-b]pyridin-2-yl)carbamate (342 mg, 1 mmol) in THF (20 mL) at 0° C. were added N,N-diisopropylethylamine (258 mg, 2 mmol) and propionyl chloride (140 mg, 1.5 mmol). The reaction solution was stirred at room temperature for 1 h before diluted with ethyl acetate (30 mL). The solution was washed with water, brine, then dried over Na₂SO₄, filtered and evaporated to give the crude product (390 mg). LC-MS (M+H)⁺=399.1.

### Step 2: N-(4-(2-aminothiazolo[5,4-b]pyridin-5-yl)phenyl)propionamide

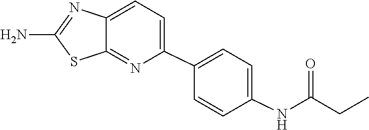 [SMILES: CCC(=O)Nc1ccc(-c2ccc3nc(N)sc3n2)cc1]

The crude tert-butyl (5-(4-propionamidophenyl)thiazolo[5,4-b]pyridin-2-yl)carbamate (390 mg) was dissolved in dichloromethane (3 mL) and cooled to 0° C. Trifluoroacetic acid (3 mL) was added and the reactions solution was stirred at room temperature for 2 h. The reaction solution was concentrated and the residue was dissolved in dichloromethane. The solution was washed with water, saturated aqueous NaHCO₃ solution, then dried over Na₂SO₄, filtered and evaporated. The crude product (250 mg, 83% for two steps) was used for next step without further purification. LC-MS (M+H)⁺=299.1.

### Step 3: 4-(5-cyano-2-methoxyphenyl)-6-methyl-N-(5-(4-propionamidophenyl)thiazolo[5,4-b]pyridin-2-yl)nicotinamide

The title compound (61 mg, 50%) was prepared in a manner similar to Example 25 step 3 from N-(4-(2-aminothiazolo[5,4-b]pyridin-5-yl)phenyl)propionamide and 4-(5-cyano-2-methoxyphenyl)-6-methylnicotinic acid. ¹H-NMR (400 MHz, d6-DMSO) δ 12.95 (s, 1H), 10.04 (s, 1H), 8.80 (s, 1H), 8.08 (td, J=23.4, 8.5 Hz, 4H), 7.96-7.88 (m, 2H), 7.73 (d, J=8.7 Hz, 2H), 7.43 (s, 1H), 7.18 (d, J=8.4 Hz, 1H), 3.60 (s, 3H), 2.60 (s, 3H), 2.36 (q, J=7.5 Hz, 2H), 1.10 (t, J=7.5 Hz, 3H). LC-MS (M+H)⁺=549.3.

### Example 42: 4-(5-cyano-2-methoxyphenyl)-N-(5-(4-(2-hydroxyacetamido)phenyl)thiazolo[5,4-b]pyridin-2-yl)-6-methylnicotinamide

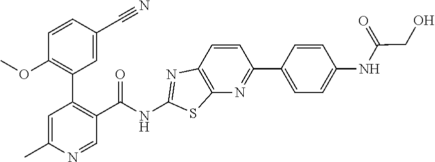 [SMILES: COc1ccc(C#N)cc1-c1cc(C)ncc1C(=O)Nc1nc2ccc(-c3ccc(NC(=O)CO)cc3)nc2s1]

### Step 1: 2-((4-(2-((tert-butoxycarbonyl)amino)thiazolo[5,4-b]pyridin-5-yl)phenyl)amino)-2-oxoethyl acetate

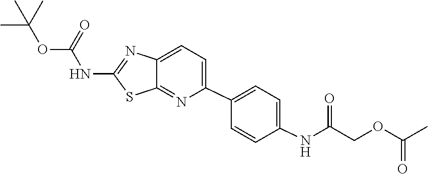 [SMILES: CC(=O)OCC(=O)Nc1ccc(-c2ccc3nc(NC(=O)OC(C)(C)C)sc3n2)cc1]

To the solution of tert-butyl (5-(4-aminophenyl)thiazolo[5,4-b]pyridin-2-yl)carbamate (342 mg, 1 mmol) in THF (20 mL) at 0° C. were added N,N-diisopropylethylamine (258 mg, 2 mmol) and 2-chloro-2-oxoethyl acetate (204 mg, 1.5 mmol). The reaction solution was stirred at room temperature for 1 h before diluted with ethyl acetate (30 mL). The mixture was washed with water, brine, then dried over Na₂SO₄, filtered and evaporated to give the crude product without further purification. LC-MS (M+H)⁺=443.1.

### Step 2: 2-((4-(2-aminothiazolo[5,4-b]pyridin-5-yl)phenyl)amino)-2-oxoethyl acetate

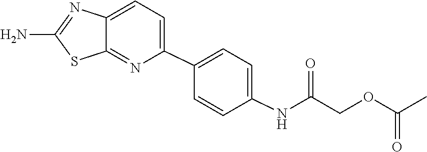 [SMILES: CC(=O)OCC(=O)Nc1ccc(-c2ccc3nc(N)sc3n2)cc1]

The crude 2-((4-(2-((tert-butoxycarbonyl)amino)thiazolo[5,4-b]pyridin-5-yl)phenyl)amino)-2-oxoethyl acetate was dissolved in dichloromethane (3 mL). The solution was cooled to 0° C. before addition of trifluoroacetic acid (3 mL). The mixture was stirred at room temperature for 2 h before concentration. The residue was dissolved in dichloromethane, washed with water, saturated aqueous NaHCO₃ solution, then dried over sodium sulfate, filtered and concentrated. The crude product (200 mg, 58% for two steps) was used for next step without further purification. LC-MS (M+H)⁺=343.1.

### Step 3: 2-((4-(2-(4-(5-cyano-2-methoxyphenyl)-6-methylnicotinamido)thiazolo[5,4-b]pyridin-5-yl)phenyl)amino)-2-oxoethyl acetate

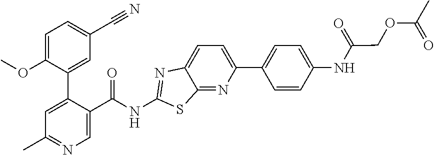 [SMILES: COc1ccc(C#N)cc1-c1cc(C)ncc1C(=O)Nc1nc2ccc(-c3ccc(NC(=O)COC(C)=O)cc3)nc2s1]

The title compound was prepared in a manner similar to that in Example 25 step 3 from 2-((4-(2-aminothiazolo[5,4-b]pyridin-5-yl)phenyl)amino)-2-oxoethyl acetate and 4-(5-cyano-2-methoxyphenyl)-6-methylnicotinic acid. crude product was used for next step without further purification. LC-MS (M+H)⁺=593.2.

### Step 4: 4-(5-cyano-2-methoxyphenyl)-N-(5-(4-(2-hydroxyacetamido)phenyl)thiazolo[5,4-b]pyridin-2-yl)-6-methylnicotinamide

To the solution of crude 2-((4-(2-(4-(5-cyano-2-methoxyphenyl)-6-methylnicotinamido)thiazolo[5,4-b]pyridin-5-yl)phenyl)amino)-2-oxoethyl acetate in THF (20 mL) was added an aqueous solution of LiOH (49 mg, 1.16 mmol). The reaction mixture was stirred at room temperature for 14 h before cooled to 0° C. and acidified by 1M HCl to pH=2. The mixture was concentrated to remove THF and solid precipitate was observed. The solid was filtered and purified by prep-HPLC (Elution: acetonitrile (0.1% formic acid)/water (0.1% formic acid): 50%-80%, v/v) to give the title compound (115 mg, 36%). ¹H-NMR (400 MHz, d6-DMSO) δ 12.95 (s, 1H), 9.84 (s, 1H), 8.80 (s, 1H), 8.26-8.00 (m, 4H), 7.97-7.69 (m, 4H), 7.44 (s, 1H), 7.18 (d, J=8.9 Hz, 1H), 5.68 (t, J=6.0 Hz, 1H), 4.03 (d, J=6.0 Hz, 2H), 3.60 (s, 3H), 2.60 (s, 3H). LC-MS (M+H)⁺=551.3.

### Example 43: 4-(5-cyano-2-methoxyphenyl)-6-methyl-N-(5-(4-(2-oxooxazolidin-3-yl)phenyl)thiazolo[5,4-b]pyridin-2-yl)nicotinamide

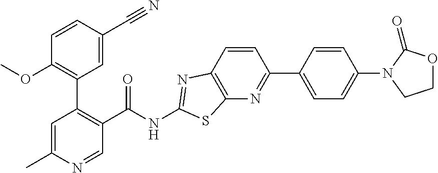 [SMILES: COc1ccc(C#N)cc1-c1cc(C)ncc1C(=O)Nc1nc2ccc(-c3ccc(N4CCOC4=O)cc3)nc2s1]

### Step 1: tert-butyl (5-(4-(((2-chloroethoxy)carbonyl)amino)phenyl)thiazolo[5,4-b]pyridin-2-yl)carbamate

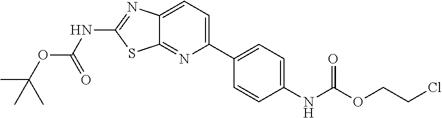 [SMILES: CC(C)(C)OC(=O)Nc1nc2ccc(-c3ccc(NC(=O)OCCCl)cc3)nc2s1]

To a solution of tert-butyl (5-(4-aminophenyl)thiazolo[5,4-b]pyridin-2-yl)carbamate (342 mg, 1 mmol) in MeCN (20 mL) at 0° C. were added K₂CO₃ (276 mg, 2 mmol) and 2-chloroethyl carbonochloridate (286 mg, 2 mmol). The reaction mixture was stirred at room temperature for 14 h before diluted with ethyl acetate (30 mL). The mixture was washed with water, brine, then dried over Na₂SO₄, filtered and evaporated. The residue was purified over silica gel by combi-flash, eluting with ethyl acetate in petroleum ether (50%, v/v) to give the title compound (300 mg, 67%). LC-MS (M+H)⁺=449.1.

### Step 2: tert-butyl (5-(4-(2-oxooxazolidin-3-yl)phenyl)thiazolo[5,4-b]pyridin-2-yl)carbamate

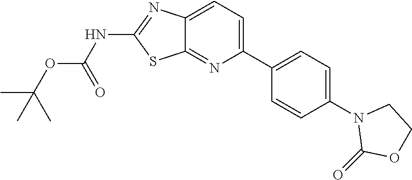 [SMILES: CC(C)(C)OC(=O)Nc1nc2ccc(-c3ccc(N4CCOC4=O)cc3)nc2s1]

To a solution of tert-butyl (5-(4-(((2-chloroethoxy)carbonyl)amino)phenyl)thiazolo[5,4-b]pyridin-2-yl)carbamate (300 mg, 0.67 mmol) in N,N-dimethylformamide (6 mL) under nitrogen was added NaH (80 mg, 2.01 mmol) at 0° C. and the resulting mixture was stirred at room temperature for 14 h before cooled to 0° C. Water was added to quench the reaction and the mixture was extracted with ethyl acetate (3×30 mL). The combined organic layers were washed with brine, dried over Na₂SO₄, filtered and evaporated. The residue was purified over silica gel by combi-flash, eluting with ethyl acetate to give the title compound (140 mg, 51%). LC-MS (M+H)⁺=413.0.

### Step 3: 3-(4-(2-aminothiazolo[5,4-b]pyridin-5-yl)phenyl)oxazolidin-2-one

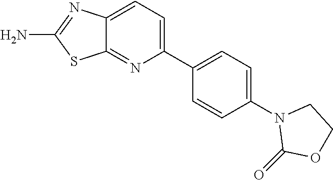 [SMILES: Nc1nc2ccc(-c3ccc(N4CCOC4=O)cc3)nc2s1]

The title compound (90 mg, 84%) was prepared in a manner similar to that in Example 1 step 6 from tert-butyl (5-(4-(2-oxooxazolidin-3-yl)phenyl)thiazolo[5,4-b]pyridin-2-yl)carbamate. LC-MS (M+H)⁺=313.1.

### Step 4: 4-(5-cyano-2-methoxyphenyl)-6-methyl-N-(5-(4-(2-oxooxazolidin-3-yl)phenyl)thiazolo[5,4-b]pyridin-2-yl)nicotinamide

The title compound (65 mg, 40%) was prepared in a manner similar to that in Example 25 step 3 from 3-(4-(2-aminothiazolo[5,4-b]pyridin-5-yl)phenyl)oxazolidin-2-one and 4-(5-cyano-2-methoxyphenyl)-6-methylnicotinic acid. ¹H-NMR (400 MHz, d6-DMSO) δ 12.95 (s, 1H), 8.82 (s, 1H), 8.15 (t, J=10.8 Hz, 3H), 8.05 (d, J=8.5 Hz, 1H), 7.91 (dd, J=11.1, 2.5 Hz, 2H), 7.71 (d, J=8.8 Hz, 2H), 7.42 (s, 1H), 7.18 (d, J=8.6 Hz, 1H), 4.57-4.38 (m, 2H), 4.13 (t, J=8.0 Hz, 2H), 3.60 (s, 3H), 2.60 (s, 3H). LC-MS (M+H)⁺=563.3.

### Example 44: 4-(5-cyano-2-methoxyphenyl)-N-(5-(4-(hydroxymethyl)phenyl)thiazolo[5,4-b]pyridin-2-yl)-6-methylnicotinamide

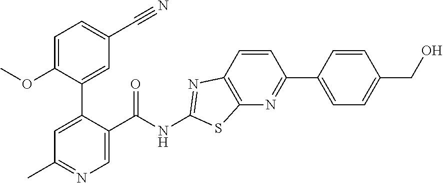 [SMILES: COc1ccc(C#N)cc1-c1cc(C)ncc1C(=O)Nc1nc2ccc(-c3ccc(CO)cc3)nc2s1]

### Step 1: tert-butyl (5-(4-(hydroxymethyl)phenyl)thiazolo[5,4-b]pyridin-2-yl)carbamate

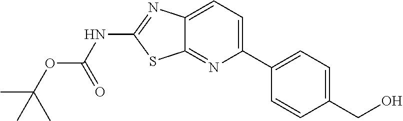 [SMILES: CC(C)(C)OC(=O)Nc1nc2ccc(-c3ccc(CO)cc3)nc2s1]

The title compound (840 mg, 52%) was prepared in a manner similar to Example 1 step 5 from tert-butyl (5-bromothiazolo[5,4-b]pyridin-2-yl)carbamate and (4-(hydroxymethyl)phenyl)boronic acid. LC-MS (M+H)⁺=358.1.

### Step 2: tert-butyl (5-(4-(((tert-butyldimethylsilyl)oxy)methyl)phenyl)thiazolo[5,4-b]pyridin-2-yl)carbamate

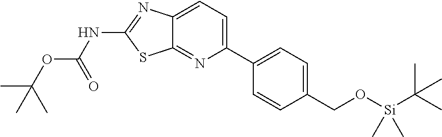 [SMILES: CC(C)(C)OC(=O)Nc1nc2ccc(-c3ccc(CO[Si](C)(C)C(C)(C)C)cc3)nc2s1]

To a solution of tert-butyl (5-(4-(hydroxymethyl)phenyl)thiazolo[5,4-b]pyridin-2-yl)carbamate (840 mg, 2.35 mmol) and Et₃N (475 mg, 4.7 mmol) in THF (50 mL) at 0° C. was added tert-butyldimethylsilyl chloride (711 mg, 4.7 mmol). The reaction solution was stirred at room temperature for 14 h before diluted with ethyl acetate (90 mL). The solution was washed with water, brine, then dried over Na₂SO₄, filtered and evaporated. The residue was purified over silica gel by combi-flash, eluting with ethyl acetate in petroleum ether (50%, v/v) to give the title compound (300 mg, 27%). ¹H-NMR (400 MHz, d6-DMSO) δ 11.97 (s, 1H), 8.09 (dd, J=8.4, 2.1 Hz, 3H), 8.01 (d, J=8.5 Hz, 1H), 7.43 (d, J=8.1 Hz, 2H), 4.78 (s, 2H), 1.53 (s, 10H), 0.92 (s, 10H), 0.10 (s, 6H). LC-MS (M+H)⁺=472.2.

### Step 3: 5-(4-(((tert-butyldimethylsilyl)oxy)methyl)phenyl)thiazolo[5,4-b]pyridin-2-amine

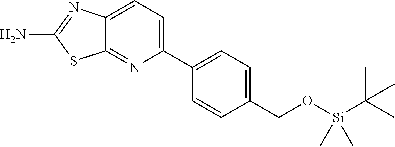 [SMILES: CC(C)(C)[Si](C)(C)OCc1ccc(-c2ccc3nc(N)sc3n2)cc1]

To a solution of tert-butyl (5-(4-(((tert-butyldimethylsilyl)oxy)methyl)phenyl)thiazolo[5,4-b]pyridin-2-yl)carbamate (300 mg, 0.64 mmol) in dichloromethane (30 mL) was added zinc chloride (1.43 g, 6.4 mmol) and the resulting mixture was stirred at room temperature for 14 h before diluted with dichloromethane. The reaction solution was washed with water and brine, then dried over sodium sulfate, filtered and evaporated to give the crude product (150 mg, 63%). LC-MS (M+H)⁺=372.2.

### Step 4: 4-(5-cyano-2-methoxyphenyl)-N-(5-(4-(hydroxymethyl)phenyl)thiazolo[5,4-b]pyridin-2-yl)-6-methylnicotinamide

The title compound (10 mg, 10%) was prepared in a manner similar to Example 25 step 3 from 5-(4-(((tert-butyldimethylsilyl)oxy)methyl)phenyl)thiazolo[5,4-b]pyridin-2-amine and 4-(5-cyano-2-methoxyphenyl)-6-methylnicotinic acid. ¹H-NMR (400 MHz, d6-DMSO) δ 12.94 (s, 1H), 8.82 (s, 1H), 8.17-7.99 (m, 4H), 7.98-7.78 (m, 2H), 7.61-7.29 (m, 3H), 7.18 (d, J=8.6 Hz, 1H), 5.26 (s, 1H), 4.57 (s, 2H), 3.60 (s, 3H), 2.59 (s, 3H). LC-MS (M+H)⁺=508.3.

### Example 45: 4-(5-cyano-2-methoxyphenyl)-6-methyl-N-(5-(4-(2-oxopyrrolidin-1-yl)phenyl)thiazolo[5,4-b]pyridin-2-yl)nicotinamide

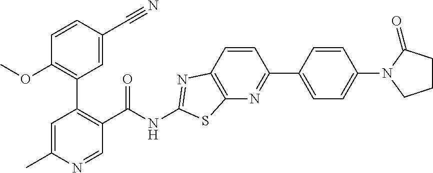 [SMILES: COc1ccc(C#N)cc1-c1cc(C)ncc1C(=O)Nc1nc2ccc(-c3ccc(N4CCCC4=O)cc3)nc2s1]

### Step 1: tert-butyl (5-(4-(4-chlorobutanamido)phenyl)thiazolo[5,4-b]pyridin-2-yl)carbamate

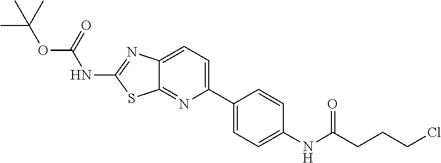 [SMILES: CC(C)(C)OC(=O)Nc1nc2ccc(-c3ccc(NC(=O)CCCCl)cc3)nc2s1]

The title compound (370 mg, 83%) was prepared in a manner similar to Example 43 step 1 from tert-butyl (5-(4-aminophenyl)thiazolo[5,4-b]pyridin-2-yl)carbamate and 4-chlorobutanoyl chloride. LC-MS (M+H)⁺=447.1

### Step 2: tert-butyl (5-(4-(2-oxopyrrolidin-1-yl)phenyl)thiazolo[5,4-b]pyridin-2-yl)carbamate

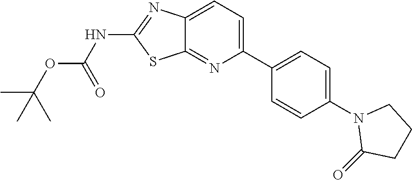 [SMILES: CC(C)(C)OC(=O)Nc1nc2ccc(-c3ccc(N4CCCC4=O)cc3)nc2s1]

The title compound (crude) was prepared in a manner similar to that in Example 43 step 2 from tert-butyl (5-(4-(4-chlorobutanamido)phenyl)thiazolo[5,4-b]pyridin-2-yl)carbamate. LC-MS (M+H)⁺=411.1.

### Step 3: 1-(4-(2-aminothiazolo[5,4-b]pyridin-5-yl)phenyl)pyrrolidin-2-one

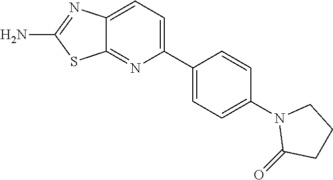 [SMILES: Nc1nc2ccc(-c3ccc(N4CCCC4=O)cc3)nc2s1]

The title compound (210 mg, 81% for two steps) was prepared in a manner similar to Example 1 step 6 from tert-butyl (5-(4-(2-oxopyrrolidin-1-yl)phenyl)thiazolo[5,4-b]pyridin-2-yl)carbamate. LC-MS (M+H)⁺=311.1.

### Step 4: 4-(5-cyano-2-methoxyphenyl)-6-methyl-N-(5-(4-(2-oxopyrrolidin-1-yl)phenyl)thiazolo[5,4-b]pyridin-2-yl)nicotinamide

The title compound (36 mg, 20%) was prepared in a manner similar to that in Example 25 step 3 from 1-(4-(2-aminothiazolo[5,4-b]pyridin-5-yl)phenyl)pyrrolidin-2-one and 4-(5-cyano-2-methoxyphenyl)-6-methylnicotinic acid. ¹H-NMR (400 MHz, d6-DMSO) δ 12.94 (s, 1H), 8.81 (s, 1H), 8.21-8.09 (m, 3H), 8.05 (d, J=8.6 Hz, 1H), 7.91 (dd, J=10.7, 2.0 Hz, 2H), 7.81 (d, J=8.8 Hz, 2H), 7.42 (s, 1H), 7.18 (d, J=8.5 Hz, 1H), 3.90 (t, J=7.0 Hz, 2H), 3.60 (s, 3H), 2.60 (s, 3H), 2.54 (t, J=7.0 Hz, 2H), 2.18-1.95 (m, 2H). LC-MS (M+H)⁺=561.3.

### Example 46: 5-((5-(4-acetamidophenyl)thiazolo[5,4-b]pyridin-2-yl)carbamoyl)-4-(2-methoxyphenyl)picolinic acid

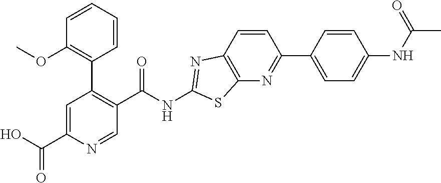 [SMILES: COc1ccccc1-c1cc(C(=O)O)ncc1C(=O)Nc1nc2ccc(-c3ccc(NC(C)=O)cc3)nc2s1]

To a solution of N-(5-(4-acetamidophenyl)thiazolo[5,4-b]pyridin-2-yl)-6-cyano-4-(2-methoxyphenyl)nicotinamide (24 mg, 0.046 mmol) in EtOH (2 mL) was added aqueous NaOH solution (10%, 2 mL). The mixture was stirred at 90° C. for 2 h under nitrogen. The reaction mixture was concentrated in vacuo. THF (2 mL) was added, followed by addition of acetyl chloride (36 mg, 0.46 mmol). The resulting mixture was stirred at room temperature for 18 h before addition of water. Then 2 N HCl solution was added until pH=3. The aqueous layer was separated from organic layer and extracted with ethyl acetate (5 mL). The combined organic layers washed with brine, dried with over sodium sulfate, filtered and concentrated under reduced pressure. The residue was purified over silica gel by combi-flash, eluting with methanol in dichloromethane (10%, v/v) to give the title compound (9.7 mg, 39%). ¹H-NMR (400 MHz, d6-DMSO) δ: 13.12 (s, 1H), 10.12 (s, 1H), 8.98 (s, 1H), 8.17 (d, J=8.6 Hz, 1H), 8.12-7.99 (m, 4H), 7.72 (d, J=8.7 Hz, 2H), 7.46 (dd, J=13.1, 7.0 Hz, 2H), 7.13 (t, J=7.3 Hz, 1H), 7.03 (d, J=8.1 Hz, 1H), 3.55 (s, 3H), 2.08 (s, 3H). LC-MS (M+H)⁺=540.3.

### Example 47: N-(5-(1H-pyrazol-5-yl)thiazolo[5,4-b]pyridin-2-yl)-5-(2-methoxyphenyl)pyridazine-4-carboxamide

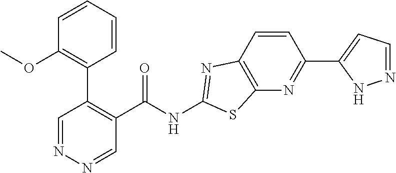 [SMILES: COc1ccccc1-c1cnncc1C(=O)Nc1nc2ccc(-c3ccn[nH]3)nc2s1]

### Step 1: 5-(1-(tetrahydro-2H-pyran-2-yl)-1H-pyrazol-5-yl)thiazolo[5,4-b]pyridin-2-amine

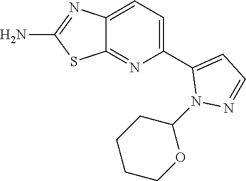 [SMILES: Nc1nc2ccc(-c3ccnn3C3CCCCO3)nc2s1]

To a stirred mixture of 5-bromothiazolo[5,4-b]pyridin-2-amine (475 mg, 2.064 mmol) and 1-(oxan-2-yl)-5-(4,4,5,5-tetramethyl-1,3,2-dioxaborolan-2-yl)pyrazole (1149 mg, 4.128 mmol) in toluene (10 mL) were added [1,1′-Bis(diphenylphosphino)ferrocene]dichloropalladium(II) (404 mg, 0.619 mmol) and K₃PO₄ (2191 mg, 10.320 mmol) at room temperature. The reaction mixture was irradiated under microwave for 2 h at 100° C. before cooled to room temperature and concentrated. The residue was purified over silica gel by combi-flash, eluting with methanol in dichloromethane (7%, v/v) to give the title compound (194 mg, 31%). LC-MS (M+H)⁺=302.2.

### Step 2: 5-(2-methoxyphenyl)-N-(5-(1-(tetrahydro-2H-pyran-2-yl)-1H-pyrazol-5-yl)thiazolo[5,4-b]pyridin-2-yl)pyridazine-4-carboxamide

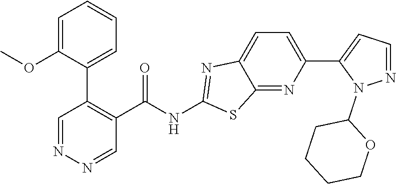 [SMILES: COc1ccccc1-c1cnncc1C(=O)Nc1nc2ccc(-c3ccnn3C3CCCCO3)nc2s1]

The title compound (28 mg, 17%) was prepared in a manner similar to that in Example 4 step 2 from 5-(1-(tetrahydro-2H-pyran-2-yl)-1H-pyrazol-5-yl)thiazolo[5,4-b]pyridin-2-amine and 5-(2-methoxyphenyl)pyridazine-4-carboxylic acid. LC-MS (M+H)⁺=514.3.

### Step 3: N-(5-(1H-pyrazol-5-yl)thiazolo[5,4-b]pyridin-2-yl)-5-(2-methoxyphenyl)pyridazine-4-carboxamide

To 5-(2-methoxyphenyl)-N-(5-(1-(tetrahydro-2H-pyran-2-yl)-1H-pyrazol-5-yl)thiazolo[5,4-b]pyridin-2-yl)pyridazine-4-carboxamide (28 mg, 0.055 mmol) in dichloromethane (2 mL) were added trifluoroacetic acid (1 mL) at 0° C. under nitrogen atmosphere. The resulting mixture was stirred for 2 h at room temperature under nitrogen atmosphere before basified to pH=9 with saturated NaHCO₃ solution. The organic layer was separated and the aqueous layer was extracted with dichloromethane (3×10 mL). The combined organic layers were washed with brine, dried over sodium sulfate, filtered and concentrated. The residue was purified by prep-HPLC under the following conditions: column, XBridge Shield RP18 OBD Column, 30×150 mm, 5 um; mobile phase, acetonitrile in water (with 10 mmol/L NH₄HCO₃ and 0.1% NH₄OH), 8% to 36% gradient in 9 min; detector, UV 254 nm. The title compound (2 mg, 10%) obtained. 1H NMR (300 MHz, d6-DMSO) δ 13.05 (brs, 1H), 9.47 (s, 1H), 9.32 (s, 1H), 8.05-7.99 (m, 2H), 7.88-7.62 (brs, 1H), 7.52-7.40 (m, 2H), 7.17-7.02 (m, 2H), 6.90-6.83 (m, 1H), 3.58 (s, 3H). LC-MS (M+H)⁺=429.9.

### Example 48: 5-(2-methoxyphenyl)-N-(5-(4-(methylsulfonamido)phenyl)thiazolo[5,4-b]pyridin-2-yl)pyridazine-4-carboxamide

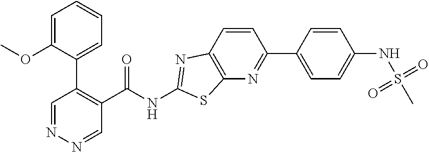 [SMILES: COc1ccccc1-c1cnncc1C(=O)Nc1nc2ccc(-c3ccc(NS(C)(=O)=O)cc3)nc2s1]

### Step 1: tert-butyl (5-(4-(methylsulfonamido)phenyl)thiazolo[5,4-b]pyridin-2-yl)carbamate

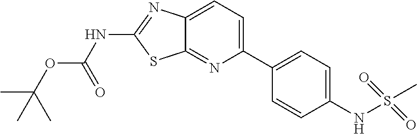 [SMILES: CC(C)(C)OC(=O)Nc1nc2ccc(-c3ccc(NS(C)(=O)=O)cc3)nc2s1]

To a mixture of tert-butyl (5-(4-aminophenyl)thiazolo[5,4-b]pyridin-2-yl)carbamate (50 mg, 0.15 mmol) in dichloromethane (5 mL) was added N,N-diisopropylethylamine (100 mg, 0.77 mmol) and methanesulfonic anhydride (50 mg, 0.3 mmol). The mixture was stirred at room temperature for 14 h before diluted with water. The mixture was extracted with dichloromethane (3×20 mL). The combined organic layers were dried over Na₂SO₄, filtered, concentrated under vacuum. The residue was purified over silica gel by prep-TLC using ethyl acetate in petroleum ether (50%, v/v) as eluent to give the title compound (50 mg, 79%). LC-MS (M+H)⁺=421.1.

### Step 2: N-(4-(2-aminothiazolo[5,4-b]pyridin-5-yl)phenyl)methanesulfonamide

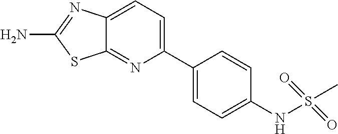 [SMILES: CS(=O)(=O)Nc1ccc(-c2ccc3nc(N)sc3n2)cc1]

The title compound (40 mg, crude) was prepared in a manner similar to that described in Example 1 step 6 from tert-butyl (5-(4-(methylsulfonamido)phenyl)thiazolo[5,4-b]pyridin-2-yl)carbamate. LC-MS (M+H)⁺=321.1.

### Step 3: 5-(2-methoxyphenyl)-N-(5-(4-(methylsulfonamido)phenyl)thiazolo[5,4-b]pyridin-2-yl)pyridazine-4-carboxamide

The title compound (10 mg, 9%) was prepared in a manner similar to that described in Example 25 step 3 from 5-(2-methoxyphenyl)pyridazine-4-carboxylic acid and N-(4-(2-aminothiazolo[5,4-b]pyridin-5-yl)phenyl)methanesulfonamide. ¹H NMR (400 MHz, d6-DMSO) δ 13.25 (brs, 1H), 9.99 (s, 1H), 9.49 (s, 1H), 9.42 (s, 1H), 8.19 (d, J=8.8 Hz, 1H), 8.11 (d, J=8.6 Hz, 2H), 8.04 (d, J=8.8 Hz, 1H), 7.58-7.44 (m, 2H), 7.33 (d, J=8.7 Hz, 2H), 7.18-7.12 (m, 1H), 7.07 (d, J=8.4 Hz, 1H), 3.56 (s, 3H), 3.06 (s, 3H). LC-MS (M+H)⁺=533.2.

### Example 49: N-(5-(4-cyanophenyl)thiazolo[5,4-b]pyridin-2-yl)-4-(2,5-dimethoxyphenyl)-6-methylnicotinamide

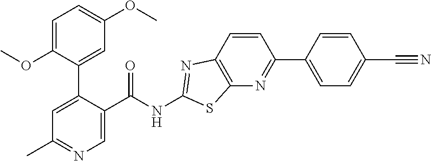 [SMILES: COc1ccc(OC)c(-c2cc(C)ncc2C(=O)Nc2nc3ccc(-c4ccc(C#N)cc4)nc3s2)c1]

### Step 1: methyl 4-(2,5-dimethoxyphenyl)-6-methylnicotinate

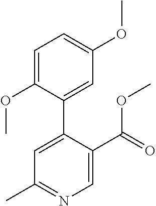 [SMILES: COC(=O)c1cnc(C)cc1-c1cc(OC)ccc1OC]

The title compound (50 mg, 31%) was prepared in a manner similar to that in Example 2 step 2 from methyl 4-chloro-6-methylnicotinate and (2,5-dimethoxyphenyl)boronic acid. LC-MS (M+H)⁺=288.2.

### Step 2: 4-(2,5-dimethoxyphenyl)-6-methylnicotinic acid

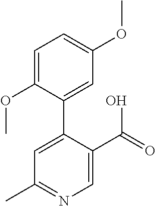 [SMILES: COc1ccc(OC)c(-c2cc(C)ncc2C(=O)O)c1]

The title compound (50 mg, 31%) was prepared in a manner similar to Example 1 step 3 from methyl 4-(2,5-dimethoxyphenyl)-6-methylnicotinate. LC-MS (M+H)⁺=274.2.

### Step 3: N-(5-(4-cyanophenyl)thiazolo[5,4-b]pyridin-2-yl)-4-(2,5-dimethoxyphenyl)-6-methylnicotinamide

The title compound (35 mg, 19%) was prepared in a manner similar to Example 25 step 3 from 4-(2,5-dimethoxyphenyl)-6-methylnicotinic acid and 4-(2-aminothiazolo[5,4-b]pyridin-5-yl)benzonitrile. ¹H-NMR (400 MHz, d6-DMSO) δ 12.99 (s, 1H), 8.74 (s, 1H), 8.34 (d, J=8.4 Hz, 2H), 8.26-8.17 (m, 2H), 7.98 (s, 1H), 7.96 (s, 1H), 7.38 (s, 1H), 7.02-6.94 (m, 2H), 6.90 (d, J=8.8 Hz, 1H), 3.78 (s, 3H), 3.46 (s, 3H), 2.59 (s, 3H). LC-MS (M+H)⁺=508.2.

### Example 50: 4-(5-cyano-2-methoxyphenyl)-N-(5-(4-isobutyramidophenyl)thiazolo[5,4-b]pyridin-2-yl)-6-methylnicotinamide

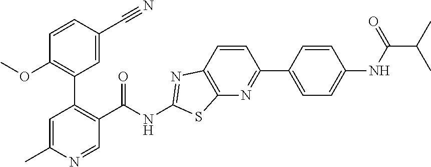 [SMILES: COc1ccc(C#N)cc1-c1cc(C)ncc1C(=O)Nc1nc2ccc(-c3ccc(NC(=O)C(C)C)cc3)nc2s1]

### Step 1: tert-butyl (5-(4-isobutyramidophenyl)thiazolo[5,4-b]pyridin-2-yl)carbamate

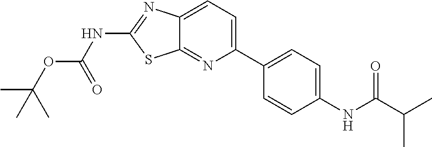 [SMILES: CC(C)C(=O)Nc1ccc(-c2ccc3nc(NC(=O)OC(C)(C)C)sc3n2)cc1]

The title compound (400 mg, 96%) was prepared in a manner similar to Example 5 step 2 from tert-butyl (5-(4-aminophenyl)thiazolo[5,4-b]pyridin-2-yl)carbamate. LC-MS (M+H)⁺=413.1.

### Step 2: N-(4-(2-aminothiazolo[5,4-b]pyridin-5-Yl)phenyl)isobutyramide

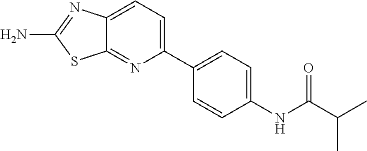 [SMILES: CC(C)C(=O)Nc1ccc(-c2ccc3nc(N)sc3n2)cc1]

The title compound (250 mg, 82.6%) was prepared in a manner similar to Example 1 step 6 from tert-butyl (5-(4-isobutyramidophenyl)thiazolo[5,4-b]pyridin-2-yl)carbamate. LC-MS (M+H)⁺=313.1.

### Step 3: 4-(5-cyano-2-methoxyphenyl)-N-(5-(4-isobutyramidophenyl)thiazolo[5,4-b]pyridin-2-yl)-6-methylnicotinamide

The title compound (62 mg, 34%) was prepared in a manner similar to that in Example 25 step 3 from 4-(5-cyano-2-methoxyphenyl)-6-methylnicotinic acid and N-(4-(2-aminothiazolo[5,4-b]pyridin-5-yl)phenyl)isobutyramide. ¹H-NMR (400 MHz, d6-DMSO) δ 12.94 (s, 1H), 9.99 (s, 1H), 8.81 (s, 1H), 8.18-7.98 (m, 4H), 7.91 (dd, J=10.6, 2.0 Hz, 2H), 7.75 (d, J=8.7 Hz, 2H), 7.42 (s, 1H), 7.18 (d, J=8.6 Hz, 1H), 3.60 (s, 3H), 2.69-2.56 (m, 4H), 1.12 (d, J=6.8 Hz, 6H). LC-MS (M+H)⁺=563.3.

### Example 51: 4-(5-cyano-2-methoxyphenyl)-N-(5-(4-isobutyramido-3-methylphenyl)thiazolo[5,4-b]pyridin-2-yl)-6-methylnicotinamide

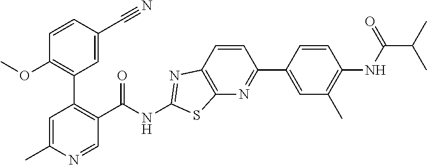 [SMILES: COc1ccc(C#N)cc1-c1cc(C)ncc1C(=O)Nc1nc2ccc(-c3ccc(NC(=O)C(C)C)c(C)c3)nc2s1]

### Step 1: tert-butyl (5-(4-amino-3-methylphenyl)thiazolo[5,4-b]pyridin-2-yl)carbamate

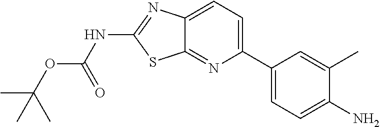 [SMILES: Cc1cc(-c2ccc3nc(NC(=O)OC(C)(C)C)sc3n2)ccc1N]

The title compound (105 mg, 70%) was prepared in a manner similar to Example 1 step 5 from tert-butyl (5-bromothiazolo[5,4-b]pyridin-2-yl)carbamate and (4-amino-3-methylphenyl)boronic acid. LC-MS (M+H)⁺=357.1.

### Step 2: tert-butyl (5-(4-isobutyramido-3-methylphenyl)thiazolo[5,4-b]pyridin-2-yl)carbamate

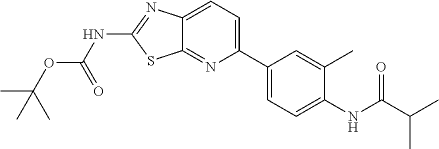 [SMILES: Cc1cc(-c2ccc3nc(NC(=O)OC(C)(C)C)sc3n2)ccc1NC(=O)C(C)C]

The title compound (200 mg, 41%) was prepared in a manner similar to Example 5 step 2 from tert-butyl (5-(4-amino-3-methylphenyl)thiazolo[5,4-b]pyridin-2-yl)carbamate. LC-MS (M+H)⁺=427.2.

### Step 3: N-(4-(2-aminothiazolo[5,4-b]pyridin-5-yl)-2-methylphenyl)isobutyramide

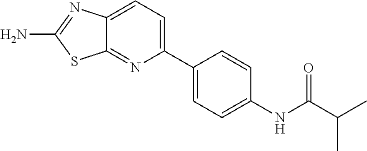 [SMILES: CC(C)C(=O)Nc1ccc(-c2ccc3nc(N)sc3n2)cc1]

The title compound (110 mg, 71%) was prepared in a manner similar to Example 1 step 6 from tert-butyl (5-(4-isobutyramido-3-methylphenyl)thiazolo[5,4-b]pyridin-2-yl)carbamate. LC-MS (M+H)⁺=327.1.

### Step 4: 4-(5-cyano-2-methoxyphenyl)-N-(5-(4-isobutyramido-3-methylphenyl)thiazolo[5,4-b]pyridin-2-yl)-6-methylnicotinamide

The title compound (30 mg, 15%) was prepared in a manner similar to that in Example 25 step 3 from 4-(5-cyano-2-methoxyphenyl)-6-methylnicotinic acid and N-(4-(2-aminothiazolo[5,4-b]pyridin-5-yl)-2-methylphenyl)isobutyramide. ¹H-NMR (400 MHz, d6-DMSO) δ 12.94 (s, 1H), 9.25 (s, 1H), 8.81 (s, 1H), 8.13 (d, J=8.5 Hz, 1H), 8.03 (d, J=8.6 Hz, 1H), 7.99 (s, 1H), 7.96-7.85 (m, 3H), 7.55 (d, J=8.4 Hz, 1H), 7.42 (s, 1H), 7.18 (d, J=8.5 Hz, 1H), 3.60 (s, 3H), 2.71 (dt, J=13.6, 6.8 Hz, 1H), 2.60 (s, 3H), 2.30 (s, 3H), 1.15 (d, J=6.9 Hz, 6H). LC-MS (M+H)⁺=577.4.

### Example 52: N-(5-(4-cyanophenyl)thiazolo[5,4-b]pyridin-2-yl)-4-(2-methoxy-5-(methylsulfonyl)phenyl)-6-methylnicotinamide

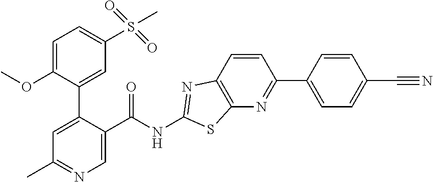 [SMILES: COc1ccc(S(C)(=O)=O)cc1-c1cc(C)ncc1C(=O)Nc1nc2ccc(-c3ccc(C#N)cc3)nc2s1]

### Step 1: 2-bromo-4-(methylsulfonyl)phenol

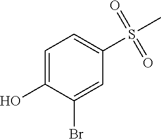

To a solution of 4-(methylsulfonyl)phenol (2.4 g, 13.94 mmol) in dichloromethane (14 mL) and methanol (14 mL) was added tetrabutylammonium tribromide (4.94 g, 15.33 mmol) and the solution was stirred at room temperature for 18 hours. Most solvent was removed under reduced pressure. ethyl acetate was added and the resulting mixture was washed with water, saturated Na₂SO₃ solution and brine. The organic layer was dried over sodium sulfate, filtered and concentrated in vacuo to afford the title compound (3.85 g, crude). LC-MS (M+H)⁺=250.9, 253.0.

### Step 2: 2-bromo-1-methoxy-4-(methylsulfonyl)benzene

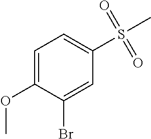

To a mixture of 2-bromo-4-(methylsulfonyl)phenol (3.85 g, 15.33 mmol) and Cs₂CO₃ (7.5 g, 23 mmol) in N,N-dimethylformamide (40 mL) was added iodomethane (2.4 g, 16.9 mmol). The reaction mixture was stirred at 25° C. for 18 h under nitrogen before diluted with ethyl acetate (50 mL). The organic solution was washed with water and brine (50 mL), dried with over sodium sulfate, filtered and concentrated under reduced pressure. The residue was purified over silica gel by combi-flash, eluting with ethyl acetate in petroleum ether (33%, v/v) to give the title compound (2 g, 49%). LC-MS (M+H)⁺=265.1, 267.1.

### Step 3: 2-(2-methoxy-5-(methylsulfonyl)phenyl)-4,4,5,5-tetramethyl-1,3,2-dioxaborolane

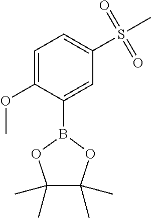 [SMILES: COc1ccc(S(C)(=O)=O)cc1B1OC(C)(C)C(C)(C)O1]

The title compound (800 mg, 68%) was prepared in a manner similar to that in Example 28 step 2 from 2-bromo-1-methoxy-4-(methylsulfonyl)benzene. LC-MS (M+H)⁺=313.1.

### Step 4: methyl 4-(2-methoxy-5-(methylsulfonyl)phenyl)-6-methylnicotinate

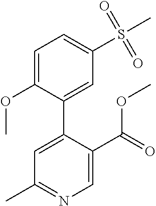 [SMILES: COC(=O)c1cnc(C)cc1-c1cc(S(C)(=O)=O)ccc1OC]

The title compound (380 mg, 99%) was prepared in a manner similar to that in Example 2 step 2 from methyl 4-chloro-6-methylnicotinate and 2-(2-methoxy-5-(methylsulfonyl)phenyl)-4,4,5,5-tetramethyl-1,3,2-dioxaborolane. LC-MS (M+H)⁺=336.1.

### Step 5: 4-(2-methoxy-5-(methylsulfonyl)phenyl)-6-methylnicotinic acid

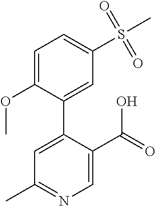 [SMILES: COc1ccc(S(C)(=O)=O)cc1-c1cc(C)ncc1C(=O)O]

The title compound (190 mg, 52%) was prepared in a manner similar to that in Example 1 step 3 from methyl 4-(2-methoxy-5-(methylsulfonyl)phenyl)-6-methylnicotinate. LC-MS (M+H)⁺=322.0.

### Step 6: N-(5-(4-cyanophenyl)thiazolo[5,4-b]pyridin-2-yl)-4-(2-methoxy-5-(methylsulfonyl)phenyl)-6-methylnicotinamide

The title compound (4.7 mg, 5%) was prepared in a manner similar to that in Example 11 step 3 from 4-(2-methoxy-5-(methylsulfonyl)phenyl)-6-methylnicotinic acid and 4-(2-aminothiazolo[5,4-b]pyridin-5-yl)benzonitrile. ¹H-NMR (400 MHz, d6-DMSO) δ:13.13 (s, 1H), 8.81 (s, 1H), 8.34 (d, J=8.4 Hz, 2H), 8.24 (q, J=8.6 Hz, 2H), 8.01-7.91 (m, 4H), 7.49 (s, 1H), 7.24 (d, J=8.7 Hz, 1H), 3.62 (s, 3H), 3.27 (s, 3H), 2.63 (s, 3H). LC-MS (M+H)⁺=556.3.

### Example 53: 4-(2-cyanophenyl)-N-(5-(4-cyanophenyl)thiazolo[5,4-b]pyridin-2-yl)-6-methylnicotinamide

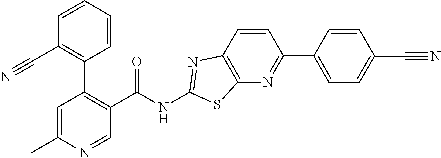 [SMILES: Cc1cc(-c2ccccc2C#N)c(C(=O)Nc2nc3ccc(-c4ccc(C#N)cc4)nc3s2)cn1]

### Step 1: methyl 4-(2-cyanophenyl)-6-methylnicotinate

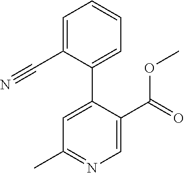 [SMILES: COC(=O)c1cnc(C)cc1-c1ccccc1C#N]

The title compound (250 mg, 37%) was prepared in a manner similar to that in Example 2 step 2 from methyl 4-chloro-6-methylnicotinate and (2-cyanophenyl)boronic acid. LC-MS (M+H)⁺=253.1.

### Step 2: 4-(2-cyanophenyl)-6-methylnicotinic acid

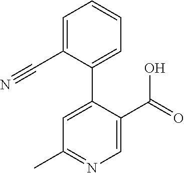 [SMILES: Cc1cc(-c2ccccc2C#N)c(C(=O)O)cn1]

The title compound (200 mg, 85%) was prepared in a manner similar to that in Example 1 step 3 from methyl 4-(2-cyanophenyl)-6-methylnicotinate. LC-MS (M+H)⁺=239.1.

### Step 3: 4-(2-cyanophenyl)-N-(5-(4-cyanophenyl)thiazolo[5,4-b]pyridin-2-yl)-6-methylnicotinamide

The title compound (20 mg, 18%) was prepared in a manner similar to that in Example 11 step 3 from 4-(2-cyanophenyl)-6-methylnicotinic acid and 4-(2-aminothiazolo[5,4-b]pyridin-5-yl)benzonitrile. ¹H-NMR (400 MHz, d6-DMSO) δ 13.39 (s, 1H), 9.03 (s, 1H), 8.34 (d, J=8.4 Hz, 2H), 8.28-8.17 (m, 2H), 8.02-7.91 (m, 3H), 7.79 (t, J=7.1 Hz, 1H), 7.63 (t, J=7.6 Hz, 1H), 7.57-7.44 (m, 2H), 2.64 (s, 3H). LC-MS (M+H)⁺=473.2.

### Example 54: N-(5-(4-cyanophenyl)thiazolo[5,4-b]pyridin-2-yl)-1,6′-dimethyl-6-oxo-1,6-dihydro-[3,4′-bipyridine]-3′-carboxamide

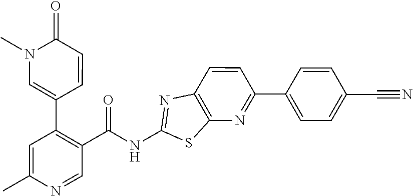 [SMILES: Cc1cc(-c2ccc(=O)n(C)c2)c(C(=O)Nc2nc3ccc(-c4ccc(C#N)cc4)nc3s2)cn1]

### Step 1: methyl 1,6′-dimethyl-6-oxo-1,6-dihydro-[3,4′-bipyridine]-3′-carboxylate

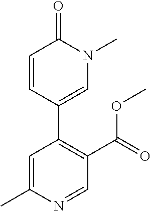

To a solution of methyl 4-chloro-6-methylnicotinate (195 mg, 1.06 mmol) and 1-methyl-5-(4,4,5,5-tetramethyl-1,3,2-dioxaborolan-2-yl)pyridin-2(1H)-one (299 mg, 1.27 mmol) in dioxane (10 mL) and water (2 mL) was added Pd(PPh₃)₄ (122 mg, 0.106 mmol) and K₂CO₃ (294 mg, 2.12 mmol). The mixture was stirred at 80° C. for 2 h under nitrogen. The reaction mixture was cooled to room temperature and diluted with ethyl acetate (50 mL), washed with brine (50 mL), dried with over sodium sulfate, filtered and concentrated under reduced pressure. The residue was purified over silica gel by combi-flash, eluting with ethyl acetate in petroleum ether (20%, v/v) to give the title compound (230 mg, 84%). LC-MS (M+H)⁺=259.0.

### Step 2: 1,6′-dimethyl-6-oxo-1,6-dihydro-[3,4′-bipyridine]-3′-carboxylic acid

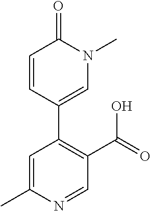 [SMILES: Cc1cc(-c2ccc(=O)n(C)c2)c(C(=O)O)cn1]

The title compound (134 mg, 61%) was prepared in a manner similar to that in Example 1 step 3 from methyl 1,6′-dimethyl-6-oxo-1,6-dihydro-[3,4′-bipyridine]-3′-carboxylate. LC-MS (M+H)⁺=245.0.

### Step 3: N-(5-(4-cyanophenyl)thiazolo[5,4-b]pyridin-2-yl)-1,6′-dimethyl-6-oxo-1,6-dihydro-[3,4′-bipyridine]-3′-carboxamide

The title compound (4.5 mg, 6%) was prepared in a manner similar to that in Example 11 step 3 from 1,6′-dimethyl-6-oxo-1,6-dihydro-[3,4′-bipyridine]-3′-carboxylic acid and 4-(2-aminothiazolo[5,4-b]pyridin-5-yl)benzonitrile. ¹H-NMR (400 MHz, d6-DMSO) δ: 13.15 (s, 1H), 8.76 (s, 1H), 8.36 (d, J=8.5 Hz, 2H), 8.28-8.18 (m, 2H), 8.03 (d, J=2.6 Hz, 1H), 7.98 (d, J=8.4 Hz, 2H), 7.45 (s, 1H), 7.37 (dd, J=9.4, 2.7 Hz, 1H), 6.39 (d, J=9.4 Hz, 1H), 3.48 (s, 3H), 2.58 (s, 3H). LC-MS (M+H)⁺=479.2.

### Example 55: N-(5-(5-chloropyridin-2-yl)thiazolo[5,4-b]pyridin-2-yl)-5-(2-methoxyphenyl)pyridazine-4-carboxamide

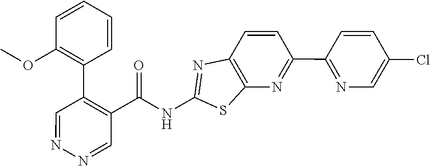 [SMILES: COc1ccccc1-c1cnncc1C(=O)Nc1nc2ccc(-c3ccc(Cl)cn3)nc2s1]

### Step 1: 5-(5-chloropyridin-2-yl)thiazolo[5,4-b]pyridin-2-amine

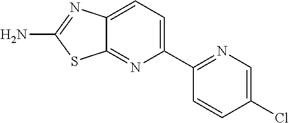 [SMILES: Nc1nc2ccc(-c3ccc(Cl)cn3)nc2s1]

To a stirred solution of 5-bromothiazolo[5,4-b]pyridin-2-amine (196 mg, 0.597 mmol) in N,N-dimethylformamide (6 mL) were added 5-chloro-2-(tributylstannyl)pyridine (240 mg, 0.597 mmol) and bis(triphenylphosphine)palladium(II) dichloride (42 mg, 0.060 mmol) at room temperature under nitrogen atmosphere. The resulting mixture was stirred for 18 h at 110° C. under nitrogen atmosphere before cooled to room temperature and quenched by addition of water (40 mL). The resulting mixture was extracted with ethyl acetate (3×30 mL). The organic phases were combined, washed with brine and dried over Na₂SO₄, then filtered and concentrated. The residue was purified over silica gel by combi-flash, eluting with ethyl acetate in petroleum ether (0% to 25%, v/v) to give the title compound (92 mg, 58%). LC-MS (M+H)⁺=263.0.

### Step 2: N-(5-(5-chloropyridin-2-yl)thiazolo[5,4-b]pyridin-2-yl)-5-(2-methoxyphenyl)pyridazine-4-carboxamide

The title compound (3 mg, 2%) was prepared in a manner similar to that in Example 1 Step 7 from 5-(5-chloropyridin-2-yl)thiazolo[5,4-b]pyridin-2-amine and 5-(2-methoxyphenyl)pyridazine-4-carboxylic acid. 1H NMR (300 MHz, d6-DMSO) δ 13.36 (brs, 1H), 9.50 (d, J=1.1 Hz, 1H), 9.37 (s, 1H), 8.77-8.70 (m, 1H), 8.49-8.39 (m, 2H), 8.22-8.01 (m, 2H), 7.56-7.42 (m, 2H), 7.19-7.02 (m, 2H), 3.57 (s, 3H). LC-MS (M+H)⁺=475.0.

### Example 56: 5-(2-methoxyphenyl)-N-(5-(4-(N-methylacetamido)phenyl)thiazolo[5,4-b]pyridin-2-yl)pyridazine-4-carboxamide

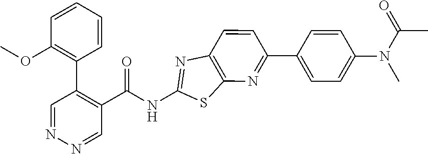 [SMILES: COc1ccccc1-c1cnncc1C(=O)Nc1nc2ccc(-c3ccc(N(C)C(C)=O)cc3)nc2s1]

### Step 1: N-methyl-N-(4-(4,4,5,5-tetramethyl-1,3,2-dioxaborolan-2-yl)phenyl)acetamide

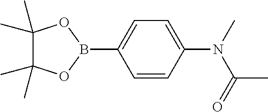 [SMILES: CC(=O)N(C)c1ccc(B2OC(C)(C)C(C)(C)O2)cc1]

To a solution of N-[4-(4,4,5,5-tetramethyl-1,3,2-dioxaborolan-2-yl)phenyl]acetamide (500 mg, 1.915 mmol) in THF (7 mL) was added NaH (114 mg, 2.873 mmol, 60%) at 0° C. under nitrogen atmosphere. The resulting mixture was stirred for 15 min at 0° C. under nitrogen atmosphere before addition of iodomethane (2175 mg, 15.320 mmol). The resulting mixture was stirred for additional 10 h at room temperature before quenched by the addition of water (20 mL). The resulting mixture was extracted with dichloromethane (2×20 mL). The combined organic layers were washed with brine, dried over Na₂SO₄, filtered and concentrated. The residue was purified over silica gel by combi-flash, eluting with ethyl acetate in petroleum ether (0% to 50%, v/v) to give the title compound (284 mg, 53%). LC-MS (M+H)⁺=276.1.

### Step 2: N-(4-(2-aminothiazolo[5,4-b]pyridin-5-yl)phenyl)-N-methylacetamide

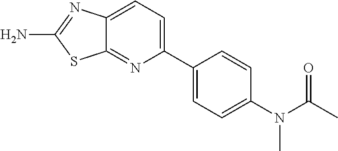 [SMILES: CC(=O)N(C)c1ccc(-c2ccc3nc(N)sc3n2)cc1]

The title compound (166 mg, 93%) was prepared in a manner similar to that in Example 1 step 5 from N-methyl-N-(4-(4,4,5,5-tetramethyl-1,3,2-dioxaborolan-2-yl)phenyl)acetamide and 5-bromothiazolo[5,4-b]pyridin-2-amine. LC-MS (M+H)⁺=299.2.

### Step 3: 5-(2-methoxyphenyl)-N-(5-(4-(N-methylacetamido)phenyl)thiazolo[5,4-b]pyridin-2-yl)pyridazine-4-carboxamide

The title compound (15 mg, 13%) was prepared in a manner similar to that in Example 4 step 2 from N-(4-(2-aminothiazolo[5,4-b]pyridin-5-yl)phenyl)-N-methylacetamide and 5-(2-methoxyphenyl)pyridazine-4-carboxylic acid. 1H NMR (300 MHz, d6-DMSO) δ 13.32 (brs, 1H), 9.51 (s, 1H), 9.42 (s, 1H), 8.26-8.06 (m, 4H), 7.63-7.39 (m, 4H), 7.21-7.04 (m, 2H), 3.57 (s, 3H), 3.22 (s, 3H), 1.88 (s, 3H). LC-MS (M+H)⁺=511.0.

### Example 57: N-(5-(4-cyanophenyl)thiazolo[5,4-b]pyridin-2-yl)-4-(5-cyclopropyl-2-methoxyphenyl)-6-methylnicotinamide

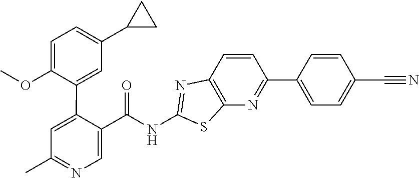 [SMILES: COc1ccc(C2CC2)cc1-c1cc(C)ncc1C(=O)Nc1nc2ccc(-c3ccc(C#N)cc3)nc2s1]

### Step 1: 4-bromo-2-chloro-1-methoxybenzene

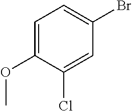

NaH (500 mg, 12.7 mmol) was added to a solution of 4-bromo-2-chlorophenol (1.8 g, 8.7 mmol) in N,N-dimethylformamide (20 mL) at 0° C. The mixture was stirred at room temperature for 30 min before addition of iodomethane (1.8 g, 12.7 mmol). The reaction mixture was stirred at room temperature for 14 h before quenched with water (50 mL). The mixture was extracted with ethyl acetate (2×30 mL). The combined organic layers were washed with water (50 mL), brine (50 mL), dried over Na₂SO₄, filtered and concentrated under vacuum. The residue was purified over silica gel by combi-flash, eluting with petroleum ether to give the title compound (1.6 g, 83%). ¹H-NMR (400 MHz, d6-DMSO) δ 7.66 (d, J=2.5 Hz, 1H), 7.50 (dd, J=8.8, 2.4 Hz, 1H), 7.12 (d, J=8.8 Hz, 1H), 3.85 (s, 3H).

### Step 2: 2-chloro-4-cyclopropyl-1-methoxybenzene

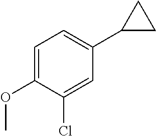

To a mixture of 4-bromo-2-chloro-1-methoxybenzene (3.1 g, 14 mmol) and cyclopropylboronic acid (1.8 g, 21 mmol) in toluene (80 mL) and water (15 mL) was added K₃PO₄ (8.9 g, 42 mmol) and (2-dicyclohexylphosphino-2′,4′,6′-triisopropyl-1,1′-biphenyl)[2-(2′-amino-1,1′-biphenyl)]palladium(II) methanesulfonate (600 mg, 0.7 mmol). The mixture was stirred at 100° C. for 14 h under nitrogen atmosphere before cooled to room temperature and diluted with water (20 mL). The mixture was extracted with ethyl acetate (2×50 mL). The combined organic layers were washed with brine, dried over Na₂SO₄, filtered and concentrated under vacuum. The residue was purified over silica gel by combi-flash, eluting with petroleum ether to give the title compound (1.8 g, 70%). ¹H-NMR (400 MHz, CDCl₃) δ 7.08 (d, J=2.1 Hz, 1H), 6.95 (dd, J=8.5, 2.2 Hz, 1H), 6.82 (d, J=8.4 Hz, 1H), 3.87 (s, 3H), 1.87-1.78 (m, 1H), 0.95-0.88 (m, 2H), 0.65-0.58 (m, 2H).

### Step 3: 2-(5-cyclopropyl-2-methoxyphenyl)-4,4,5,5-tetramethyl-1,3,2-dioxaborolane

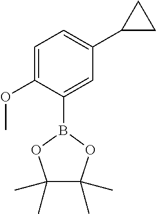 [SMILES: COc1ccc(C2CC2)cc1B1OC(C)(C)C(C)(C)O1]

To a solution of 2-chloro-4-cyclopropyl-1-methoxybenzene (1.8 g, 10 mmol) and 4,4,4′,4′,5,5,5′,5′-octamethyl-2,2′-bi(1,3,2-dioxaborolane) (3.8 g, 15 mmol) in dioxane (30 mL) was added KOAc (2.9 g, 30 mmol) and (2-dicyclohexylphosphino-2′,4′,6′-triisopropyl-1,1′-biphenyl)[2-(2′-amino-1,1′-biphenyl)]palladium(II) methanesulfonate (390 mg, 0.5 mmol). The mixture was stirred at 80° C. for 14 h under nitrogen atmosphere before cooled to room temperature and diluted with water (20 mL). The mixture was extracted with ethyl acetate (2×50 mL). The combined organic layers were washed with brine, dried over Na₂SO₄, filtered and concentrated under vacuum. The residue was purified over silica gel by combi-flash, eluting with petroleum ether to give the title compound (0.5 g, 18%). LC-MS (M+H)⁺=275.2.

### Step 4: methyl 4-(5-cyclopropyl-2-methoxyphenyl)-6-methylnicotinate

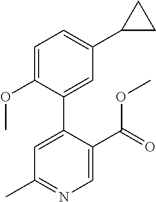 [SMILES: COC(=O)c1cnc(C)cc1-c1cc(C2CC2)ccc1OC]

The title compound (370 mg, 95%) was prepared in a manner similar to that in Example 2 step 2 from 2-(5-cyclopropyl-2-methoxyphenyl)-4,4,5,5-tetramethyl-1,3,2-dioxaborolane and methyl 4-chloro-6-methylnicotinate. LC-MS (M+H)⁺=298.2.

### Step 5: 4-(5-cyclopropyl-2-methoxyphenyl)-6-methylnicotinic acid

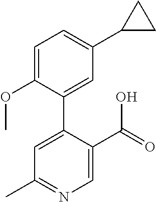 [SMILES: COc1ccc(C2CC2)cc1-c1cc(C)ncc1C(=O)O]

The title compound (200 mg, 70%) was prepared in a manner similar to that in Example 1 step 3 from methyl 4-(5-cyclopropyl-2-methoxyphenyl)-6-methylnicotinate. LC-MS (M+H)⁺=284.2.

### Step 6: N-(5-(4-cyanophenyl)thiazolo[5,4-b]pyridin-2-yl)-4-(5-cyclopropyl-2-methoxyphenyl)-6-methylnicotinamide

The title compound (15 mg, 14%) was prepared in a manner similar to that in Example 11 step 3 from 4-(5-cyclopropyl-2-methoxyphenyl)-6-methylnicotinic acid and 4-(2-aminothiazolo[5,4-b]pyridin-5-yl)benzonitrile. ¹H-NMR (400 MHz, d6-DMSO) δ 12.98 (s, 1H), 8.72 (s, 1H), 8.34 (d, J=8.4 Hz, 2H), 8.26-8.18 (m, 2H), 7.97 (d, J=8.3 Hz, 2H), 7.35 (s, 1H), 7.12-7.07 (m, 2H), 6.86 (d, J=9.1 Hz, 1H), 3.47 (s, 3H), 2.59 (s, 3H), 1.99-1.90 (m, 1H), 0.98-0.88 (m, 2H), 0.74-0.64 (m, 2H). LC-MS (M+H)⁺=518.3.

### Example 58: 6-chloro-4-(5-cyano-2-methoxyphenyl)-N-(5-(4-morpholinophenyl)thiazolo[5,4-b]pyridin-2-yl)nicotinamide

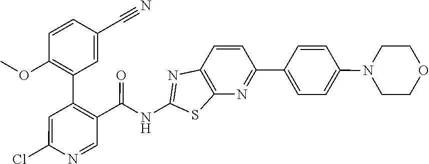 [SMILES: COc1ccc(C#N)cc1-c1cc(Cl)ncc1C(=O)Nc1nc2ccc(-c3ccc(N4CCOCC4)cc3)nc2s1]

### Step 1: tert-butyl (5-(4-morpholinophenyl)thiazolo[5,4-b]pyridin-2-yl)carbamate

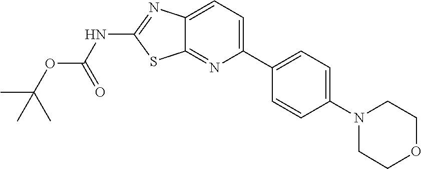 [SMILES: CC(C)(C)OC(=O)Nc1nc2ccc(-c3ccc(N4CCOCC4)cc3)nc2s1]

The reaction mixture of tert-butyl (5-bromothiazolo[5,4-b]pyridin-2-yl)carbamate (0.66 g, 2 mmol), (4-morpholinophenyl)boronic acid (0.5 g, 2.4 mmol), [1,1′-Bis(diphenylphosphino)ferrocene]dichloropalladium(II) (0.16 g, 0.2 mmol) and K₂CO₃ (0.69 g, 5 mmol) in dioxane (7 mL) under nitrogen was stirred at 100° C. for 14 h before cooled to room temperature. The mixture was filtered and the filtrate was evaporated. The residue was purified over silica gel by combi-flash, eluting with ethyl acetate in petroleum ether (50%, v/v) to give the title compound (0.5 g, 61%). LC-MS (M+H)⁺=413.2.

### Step 2: 5-(4-morpholinophenyl)thiazolo[5,4-b]pyridin-2-amine

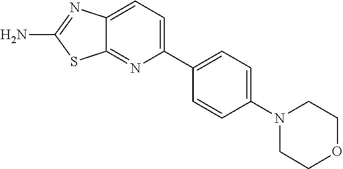 [SMILES: Nc1nc2ccc(-c3ccc(N4CCOCC4)cc3)nc2s1]

The title compound (260 mg, 63%) was prepared in a manner similar to that in Example 1 step 6 from tert-butyl (5-(4-morpholinophenyl)thiazolo[5,4-b]pyridin-2-yl)carbamate.

### Step 3: 6-chloro-4-(5-cyano-2-methoxyphenyl)-N-(5-(4-morpholinophenyl)thiazolo[5,4-b]pyridin-2-yl)nicotinamide

The title compound (20 mg, 15.6%) was prepared in a manner similar to Example 11 of Step 3 from 6-chloro-4-(5-cyano-2-methoxyphenyl)nicotinic acid and 5-(4-morpholinophenyl)thiazolo[5,4-b]pyridin-2-amine. ¹H-NMR (400 MHz, d6-DMSO) δ 13.06 (s, 1H), 8.79 (s, 1H), 8.12 (d, J=8.4 Hz, 1H), 8.03-7.93 (m, 5H), 7.76 (s, 1H), 7.20 (d, J=8.7 Hz, 1H), 7.05 (d, J=9.0 Hz, 2H), 3.76 (t, J=4.6 Hz, 4H), 3.61 (s, 3H), 3.21 (t, J=4.8 Hz, 4H). LC-MS (M+H)⁺=583.3.

### Example 59: 4-(5-cyano-2-methoxyphenyl)-6-methyl-N-(5-(4-morpholinophenyl)thiazolo[5,4-b]pyridin-2-yl)nicotinamide

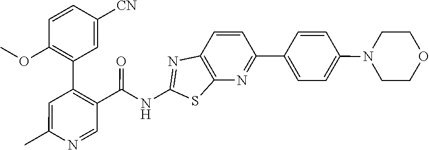 [SMILES: COc1ccc(C#N)cc1-c1cc(C)ncc1C(=O)Nc1nc2ccc(-c3ccc(N4CCOCC4)cc3)nc2s1]

The title compound (25 mg, 17%) was prepared in a manner similar to that in Example 11 of Step 3 from 4-(5-cyano-2-methoxyphenyl)-6-methylnicotinic acid and 5-(4-morpholinophenyl)thiazolo[5,4-b]pyridin-2-amine. ¹H-NMR (400 MHz, d6-DMSO) δ 12.89 (s, 1H), 8.80 (s, 1H), 8.07-7.90 (m, 6H), 7.42 (s, 1H), 7.18 (d, J=8.5 Hz, 1H), 7.05 (d, J=8.8 Hz, 2H), 3.76 (t, J=4.6 Hz, 4H), 3.60 (s, 3H), 3.21 (t, J=4.6 Hz, 4H), 2.60 (s, 3H). LC-MS (M+H)⁺=563.3.

### Example 60: 4-(5-cyano-2-methoxyphenyl)-6-methyl-N-(5-morpholinothiazolo[5,4-b]pyridin-2-yl)nicotinamide

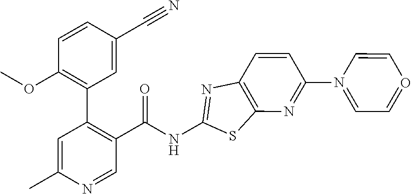

### Step 1: tert-butyl (5-morpholinothiazolo[5,4-b]pyridin-2-yl)carbamate

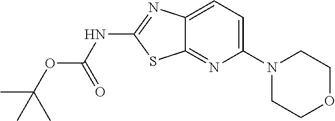 [SMILES: CC(C)(C)OC(=O)Nc1nc2ccc(N3CCOCC3)nc2s1]

The reaction mixture of tert-butyl (5-bromothiazolo[5,4-b]pyridin-2-yl)carbamate (0.33 g, 1 mmol), morpholine (0.13 g, 1.5 mmol), tris(dibenzylideneacetone)dipalladium(0) (92 mg, 0.1 mmol), 4,5-bis(diphenylphosphino)-9,9-dimethylxanthene (116 mg, 0.2 mmol) and sodium tert-butoxide (190 mg, 2 mmol) in toluene/t-BuOH (3 mL, v/v 2/1) was stirred under nitrogen atmosphere at 100° C. for 14 h before cooled to room temperature. The mixture was filtered and the filtrate was concentrated. The residue was purified over silica gel by combi-flash, eluting with ethyl acetate in petroleum ether (50%, v/v) to give the title compound (0.13 g, 39%). LC-MS (M+H)⁺=337.2.

### Step 2: 5-morpholinothiazolo[5,4-b]pyridin-2-amine

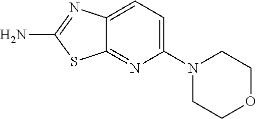 [SMILES: Nc1nc2ccc(N3CCOCC3)nc2s1]

The title compound (130 mg, crude) was prepared in a manner similar to that in Example 1 step 6 from tert-butyl (5-morpholinothiazolo[5,4-b]pyridin-2-yl)carbamate. LC-MS (M+H)⁺=237.2.

### Step 3: 4-(5-cyano-2-methoxyphenyl)-6-methyl-N-(5-morpholinothiazolo[5,4-b]pyridin-2-yl)nicotinamide

The title compound (3 mg, 3.1%) was prepared in a manner similar to Example 11 of Step 3 from 4-(5-cyano-2-methoxyphenyl)-6-methylnicotinic acid and 5-morpholinothiazolo[5,4-b]pyridin-2-amine. ¹H-NMR (400 MHz, d6-DMSO) δ 12.63 (s, 1H), 8.75 (s, 1H), 7.92-7.85 (m, 3H), 7.40 (s, 1H), 7.17 (d, J=8.5 Hz, 1H), 6.99 (d, J=9.1 Hz, 1H), 3.72 (t, J=4.6 Hz, 4H), 3.58 (s, 3H), 3.47 (t, J=4.6 Hz, 4H), 2.58 (s, 3H). LC-MS (M+H)⁺=487.3.

### Example 61: N-(5-(4-cyanophenyl)thiazolo[5,4-b]pyridin-2-yl)-4-(1H-indazol-4-yl)-6-methylnicotinamide

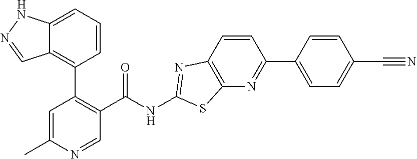 [SMILES: Cc1cc(-c2cccc3[nH]ncc23)c(C(=O)Nc2nc3ccc(-c4ccc(C#N)cc4)nc3s2)cn1]

### Step 1: 4-chloro-N-(5-(4-cyanophenyl)thiazolo[5,4-b]pyridin-2-yl)-6-methylnicotinamide

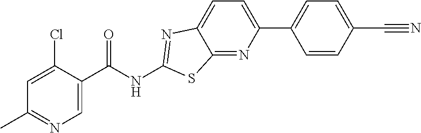 [SMILES: Cc1cc(Cl)c(C(=O)Nc2nc3ccc(-c4ccc(C#N)cc4)nc3s2)cn1]

The title compound (210 mg, 65%) was prepared in a manner similar to that in Example 2 step 4 from 4-chloro-6-methylnicotinic acid and 4-(2-aminothiazolo[5,4-b]pyridin-5-yl)benzonitrile. LC-MS (M+H)⁺=406.0.

### Step 2: N-(5-(4-cyanophenyl)thiazolo[5,4-b]pyridin-2-yl)-4-(1H-indazol-4-yl)-6-methylnicotinamide

The title compound (16 mg, 22%) was prepared in a manner similar to that in Example 2 step 2 from 4-chloro-N-(5-(4-cyanophenyl)thiazolo[5,4-b]pyridin-2-yl)-6-methylnicotinamide and (1H-indazol-4-yl)boronic acid. ¹H-NMR (400 MHz, d6-DMSO) δ: 13.23 (s, 1H), 13.17 (s, 1H), 8.87 (s, 1H), 8.32 (d, J=8.4 Hz, 2H), 8.19 (s, 2H), 7.96 (d, J=8.4 Hz, 3H), 7.61-7.53 (m, 2H), 7.44-7.36 (m, 1H), 7.11 (d, J=7.0 Hz, 1H), 2.64 (s, 3H). LC-MS (M+H)⁺=488.2.

### Example 62: N-(5-(4-chlorophenethyl)thiazolo[5,4-b]pyridin-2-yl)-5-(2-methoxyphenyl)pyridazine-4-carboxamide

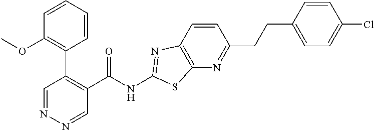 [SMILES: COc1ccccc1-c1cnncc1C(=O)Nc1nc2ccc(CCc3ccc(Cl)cc3)nc2s1]

### Step 1: tert-butyl (5-((4-chlorophenyl)ethynyl)thiazolo[5,4-b]pyridin-2-yl)carbamate

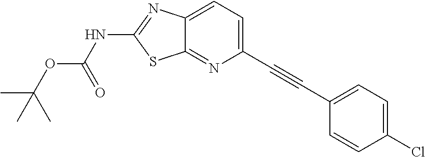 [SMILES: CC(C)(C)OC(=O)Nc1nc2ccc(C#Cc3ccc(Cl)cc3)nc2s1]

To a solution of tert-butyl (5-bromothiazolo[5,4-b]pyridin-2-yl)carbamate (605 mg, 1.830 mmol) and 1-chloro-4-ethynylbenzene (250 mg, 1.830 mmol) in acetonitrile (16 mL) and methanol (8 mL) was added N,N-diisopropylethylamine (525 mg, 4.063 mmol,), triphenylphosphine (105 mg, 0.403 mmol), CuI (38 mg, 0.201 mmol), and Pd(OAc)₂ (45 mg, 0.201 mmol) at room temperature. The resulting mixture was stirred at 80° C. for 16 h under nitrogen atmosphere before cooled to room temperature. The resulting mixture was filtered and the filter cake was washed with dichloromethane (3×10 mL). The filtrate was concentrated under reduced pressure and the residue was purified over silica gel by combi-flash, eluting with ethyl acetate in petroleum ether (0% to 50%, v/v) to give the title compound (268 mg, 38%). LC-MS (M+H)⁺=386.0.

### Step 2: tert-butyl (5-(4-chlorophenethyl)thiazolo[5,4-b]pyridin-2-yl)carbamate

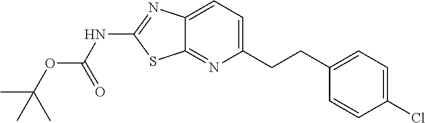 [SMILES: CC(C)(C)OC(=O)Nc1nc2ccc(CCc3ccc(Cl)cc3)nc2s1]

To a solution of tert-butyl (5-((4-chlorophenyl)ethynyl)thiazolo[5,4-b]pyridin-2-yl)carbamate (270 mg, 0.7 mmol) in methanol (2 mL) was added 10 wt. % Pd/C (0.15 g, 0.14 mmol) under nitrogen atmosphere. The mixture was hydrogenated at room temperature for 2 h under hydrogen atmosphere using a hydrogen balloon. The reaction mixture was filtered through a Celite pad and the filtrate was concentrated under reduced pressure. The residue was purified over silica gel by combi-flash, eluting with ethyl acetate in petroleum ether (0% to 50%, v/v) to give the title compound (254 mg, 93%). LC-MS (M+H)⁺=390.0.

### Step 3: 5-(4-chlorophenethyl)thiazolo[5,4-b]pyridin-2-amine

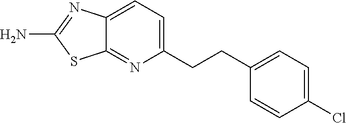 [SMILES: Nc1nc2ccc(CCc3ccc(Cl)cc3)nc2s1]

The title compound (105 mg, 55%) was prepared in a manner similar to that in Example 1 step 6 from tert-butyl (5-(4-chlorophenethyl)thiazolo[5,4-b]pyridin-2-yl)carbamate. LC-MS (M+H)⁺=290.0.

### Step 4: N-(5-(4-chlorophenethyl)thiazolo[5,4-b]pyridin-2-yl)-5-(2-methoxyphenyl)pyridazine-4-carboxamide

The title compound (8 mg, 19%) was prepared in a manner similar to that in Example 4 step 2 from 5-(4-chlorophenethyl)thiazolo[5,4-b]pyridin-2-amine and 5-(2-methoxyphenyl)pyridazine-4-carboxylic acid. 1H NMR (400 MHz, d6-DMSO) δ 13.18 (brs, 1H), 9.47 (s, 1H), 9.40 (s, 1H), 8.03 (d, J=8.3 Hz, 1H), 7.55-7.44 (m, 2H), 7.37 (d, J=8.3 Hz, 1H), 7.34-7.24 (m, 4H), 7.18-7.01 (m, 2H), 3.55 (s, 3H), 3.18-3.09 (m, 2H), 3.09-2.99 (m, 2H). LC-MS (M+H)⁺=502.0.

### Example 63: N-(5-((4-chlorophenyl)ethynyl)thiazolo[5,4-b]pyridin-2-yl)-5-(2-methoxyphenyl)pyridazine-4-carboxamide

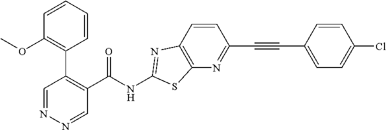 [SMILES: COc1ccccc1-c1cnncc1C(=O)Nc1nc2ccc(C#Cc3ccc(Cl)cc3)nc2s1]

### Step 1: 5-((4-chlorophenyl)ethynyl)thiazolo[5,4-b]pyridin-2-amine

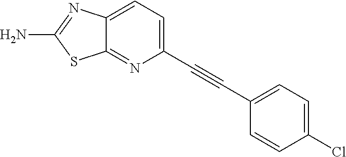 [SMILES: Nc1nc2ccc(C#Cc3ccc(Cl)cc3)nc2s1]

The title compound (166 mg, 92%) was prepared in a manner similar to that in Example 1 step 6 from tert-butyl (5-((4-chlorophenyl)ethynyl)thiazolo[5,4-b]pyridin-2-yl)carbamate. LC-MS (M+H)⁺=285.9.

### Step 2: N-(5-((4-chlorophenyl)ethynyl)thiazolo[5,4-b]pyridin-2-yl)-5-(2-methoxyphenyl)pyridazine-4-carboxamide

The title compound (21 mg, 16%) was prepared in a manner similar to that in Example 1 step 7 from 5-((4-chlorophenyl)ethynyl)thiazolo[5,4-b]pyridin-2-amine and 5-(2-methoxyphenyl)pyridazine-4-carboxylic acid. 1H NMR (300 MHz, d6-DMSO) δ 13.42 (brs, 1H), 9.50 (s, 1H), 9.43 (s, 1H), 8.19 (d, J=8.4 Hz, 1H), 7.77 (d, J=8.4 Hz, 1H), 7.73-7.62 (m, 2H), 7.60-7.44 (m, 4H), 7.21-7.02 (m, 2H), 3.56 (s, 3H). LC-MS (M+H)⁺=498.0.

### Example 64: N-(5-(4-cyanophenyl)thiazolo[5,4-b]pyridin-2-yl)-4-(2-(hydroxymethyl)phenyl)-6-methylnicotinamide

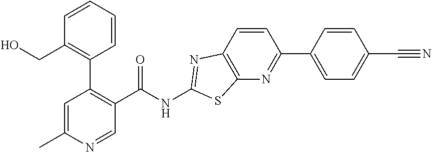 [SMILES: Cc1cc(-c2ccccc2CO)c(C(=O)Nc2nc3ccc(-c4ccc(C#N)cc4)nc3s2)cn1]

### Step 1: 2-((2-bromobenzyl)oxy)tetrahydro-2H-pyran

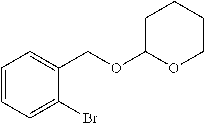 [SMILES: Brc1ccccc1COC1CCCCO1]

4-methylbenzenesulfonic acid (78 mg, 0.41 mmol) was added to a solution of (2-bromophenyl)methanol (1.54 g, 8.2 mmol) and 3,4-dihydro-2H-pyran (827 mg, 9.85 mmol) in dichloromethane (50 mL). The resulting solution was stirred at room temperature for 14 h. The reaction solution was washed with water (10 mL). The organic layer was dried over Na₂SO₄, filtered and concentrated. The residue was purified over prep-TLC using ethyl acetate in petroleum ether (17%) as eluent to give the title compound (2.22 g, 99%).

### Step 2: 4,4,5,5-tetramethyl-2-(2-(((tetrahydro-2H-pyran-2-yl)oxy)methyl)phenyl)-1,3,2-dioxaborolane

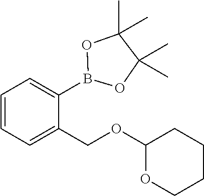 [SMILES: CC1(C)OB(c2ccccc2COC2CCCCO2)OC1(C)C]

The title compound (2.06 g, 79%) was prepared in a manner similar to that in Example 28 step 2 from 2-((2-bromobenzyl)oxy)tetrahydro-2H-pyran.

### Step 3: methyl 6-methyl-4-(2-(((tetrahydro-2H-pyran-2-yl)oxy)methyl)phenyl)nicotinate

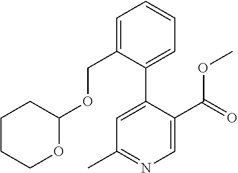 [SMILES: COC(=O)c1cnc(C)cc1-c1ccccc1COC1CCCCO1]

The title compound (123 mg, 27%) was prepared in a manner similar to that in Example 2 step 2 from methyl 4-chloro-6-methylnicotinate and 4,4,5,5-tetramethyl-2-(2-(((tetrahydro-2H-pyran-2-yl)oxy)methyl)phenyl)-1,3,2-dioxaborolane. LC-MS (M+H)⁺=342.3.

### Step 4: 6-methyl-4-(2-(((tetrahydro-2H-pyran-2-yl)oxy)methyl)phenyl)nicotinic acid

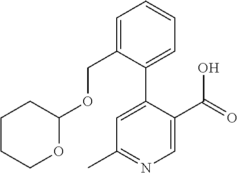 [SMILES: Cc1cc(-c2ccccc2COC2CCCCO2)c(C(=O)O)cn1]

The title compound (100 mg, 85%) was prepared in a manner similar to that in Example 1 step 3 from methyl 6-methyl-4-(2-(((tetrahydro-2H-pyran-2-yl)oxy)methyl)phenyl)nicotinate. LC-MS (M+H)⁺=328.1.

### Step 5: N-(5-(4-cyanophenyl)thiazolo[5,4-b]pyridin-2-yl)-6-methyl-4-(2-(((tetrahydro-2H-pyran-2-yl)oxy)methyl)phenyl)nicotinamide

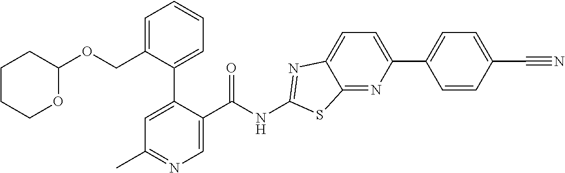 [SMILES: Cc1cc(-c2ccccc2COC2CCCCO2)c(C(=O)Nc2nc3ccc(-c4ccc(C#N)cc4)nc3s2)cn1]

The title compound (62 mg, 36%) was prepared in a manner similar to that described in Example 11 step 3 from 6-methyl-4-(2-(((tetrahydro-2H-pyran-2-yl)oxy)methyl)phenyl)nicotinic acid and 4-(2-aminothiazolo[5,4-b]pyridin-5-yl)benzonitrile. LC-MS (M+H)⁺=562.2.

### Step 6: N-(5-(4-cyanophenyl)thiazolo[5,4-b]pyridin-2-yl)-4-(2-(hydroxymethyl)phenyl)-6-methylnicotinamide

To a solution of N-(5-(4-cyanophenyl)thiazolo[5,4-b]pyridin-2-yl)-6-methyl-4-(2-(((tetrahydro-2H-pyran-2-yl)oxy)methyl)phenyl)nicotinamide (62 mg, 0.11 mmol) in methanol (5 mL) and dichloromethane (5 mL) was added 4-methylbenzenesulfonic acid (2.1 mg, 0.01 mmol). The resulting solution was stirred at room temperature for 14 h before washed with water (5 mL). The aqueous layer was extracted with dichloromethane (3×5 mL). The combined dichloromethane layers were dried, filtered and concentrated. The residue was purified by Prep-TLC using methanol in dichloromethane (5%, v/v) to give the title compound (40 mg, 75%). ¹H-NMR (400 MHz, d6-DMSO) δ 12.90 (s, 1H), 8.84 (s, 1H), 8.33 (d, J=8.3 Hz, 2H), 8.27-8.15 (m, 2H), 7.97 (d, J=8.3 Hz, 2H), 7.56 (d, J=7.5 Hz, 1H), 7.38 (t, J=7.4 Hz, 1H), 7.33 (s, 1H), 7.28 (t, J=7.3 Hz, 1H), 7.10 (d, J=7.6 Hz, 1H), 5.43 (s, 1H), 4.39 (s, 2H), 2.59 (s, 3H). LC-MS (M+H)⁺=478.4.

### Example 65: 4-(5-cyano-2-methoxyphenyl)-6-methyl-N-(5-(2-morpholinoethoxy)thiazolo[5,4-b]pyridin-2-yl)nicotinamide

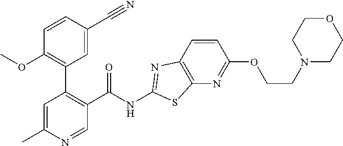 [SMILES: COc1ccc(C#N)cc1-c1cc(C)ncc1C(=O)Nc1nc2ccc(OCCN3CCOCC3)nc2s1]

### Step 1: tert-butyl (5-(2-morpholinoethoxy)thiazolo[5,4-b]pyridin-2-yl)carbamate

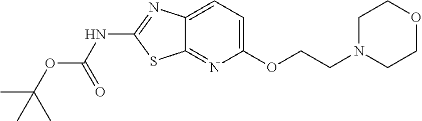 [SMILES: CC(C)(C)OC(=O)Nc1nc2ccc(OCCN3CCOCC3)nc2s1]

To a solution of 2-morpholinoethan-1-ol (330 mg, 1 mmol) in anhydrous N,N-dimethylformamide (50 mL) was added NaH (88 mg, 60%, 2.2 mmol) slowly at 0° C. followed by addition of tert-butyl (5-bromothiazolo[5,4-b]pyridin-2-yl)carbamate. The resulting mixture was stirred at 100° C. for 16 h before cooled to room temperature and addition of water (20 mL). The mixture was extracted with ethyl acetate (3×20 mL). The combined organic layers were dried over Na₂SO₄, filtered and concentrated. The residue was purified over silica gel by combi-flash, eluting with ethyl acetate in petroleum ether (50%, v/v) to give the title compound (50 mg, 13%). LC-MS (M+H)⁺=381.1.

### Step 2: 5-(2-morpholinoethoxy)thiazolo[5,4-b]pyridin-2-amine

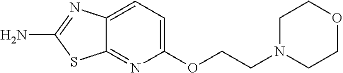 [SMILES: Nc1nc2ccc(OCCN3CCOCC3)nc2s1]

To a reaction solution of tert-butyl (5-(2-morpholinoethoxy)thiazolo[5,4-b]pyridin-2-yl)carbamate (50 mg, 0.13 mmol) in dichloromethane (3 mL) was added trifluoroacetic acid (3 mL) and the reaction mixture was stirred at room temperature for 2 h before concentration. The residue was treated with saturated aqueous NaHCO₃ solution. The precipitate was filtered, washed with water and dried to give the title compound (36 mg, crude) which was used straightly in the next step without further purification. LC-MS (M+H)⁺=281.1.

### Step 3: 4-(5-cyano-2-methoxyphenyl)-6-methyl-N-(5-(2-morpholinoethoxy)thiazolo[5,4-b]pyridin-2-yl)nicotinamide

The title compound (1.5 mg, 2%) was prepared in a manner similar to Example 25 step 3 from 4-(5-cyano-2-methoxyphenyl)-6-methylnicotinic acid and 5-(2-morpholinoethoxy)thiazolo[5,4-b]pyridin-2-amine. ¹H-NMR (400 MHz, d6-DMSO) δ 8.74 (s, 1H), 7.94 (d, J=8.8 Hz, 1H), 7.81-7.78 (m, 2H), 7.42 (s, 1H), 7.12 (d, J=9.3 Hz, 1H), 6.88 (d, J=8.9 Hz, 1H), 4.52 (t, J=5.6 Hz, 2H), 3.75-3.69 (m, 4H), 3.67 (s, 3H), 2.84 (t, J=5.6 Hz, 2H), 2.67 (s, 3H), 2.65-2.57 (m, 4H). LC-MS (M+H)⁺=531.1.

### Example 66: N-(5-(4-cyanophenyl)thiazolo[5,4-b]pyridin-2-yl)-4-(2-methoxy-5-(methoxymethyl)phenyl)-6-methylnicotinamide

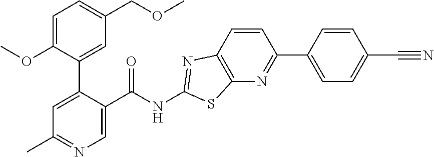 [SMILES: COCc1ccc(OC)c(-c2cc(C)ncc2C(=O)Nc2nc3ccc(-c4ccc(C#N)cc4)nc3s2)c1]

The title compound (11 mg, 7%) was prepared in a manner similar to that in Example 25 step 3 from 4-(2-methoxy-5-(methoxymethyl)phenyl)-6-methylnicotinic acid and 4-(2-aminothiazolo[5,4-b]pyridin-5-yl)benzonitrile. ¹H-NMR (400 MHz, d6-DMSO) δ 13.03 (s, 1H), 8.74 (s, 1H), 8.34 (d, J=8.4 Hz, 2H), 8.27-8.18 (m, 2H), 7.97 (d, J=8.4 Hz, 2H), 7.35-7.34 (m, 3H), 6.96 (d, J=9.0 Hz, 1H), 4.42 (s, 2H), 3.51 (s, 3H), 3.30 (s, 3H), 2.59 (s, 3H). LC-MS (M+H)⁺=522.2.

### Example 67: 4-(5-cyano-2-methoxyphenyl)-6-methyl-N-(5-(tetrahydro-2H-pyran-4-yl)thiazolo[5,4-b]pyridin-2-yl)nicotinamide

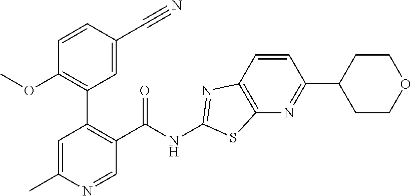 [SMILES: COc1ccc(C#N)cc1-c1cc(C)ncc1C(=O)Nc1nc2ccc(C3CCOCC3)nc2s1]

### Step 1: tert-butyl (5-(3,6-dihydro-2H-pyran-4-yl)thiazolo[5,4-b]pyridin-2-yl)carbamate

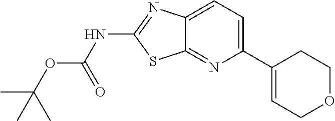 [SMILES: CC(C)(C)OC(=O)Nc1nc2ccc(C3=CCOCC3)nc2s1]

The title compound (200 mg, 79%) was prepared in a manner similar to that in Example 2 step 2 from tert-butyl (5-bromothiazolo[5,4-b]pyridin-2-yl)carbamate and 2-(3,6-dihydro-2H-pyran-4-yl)-4,4,5,5-tetramethyl-1,3,2-dioxaborolane. LC-MS (M+H)⁺=334.1.

### Step 2: tert-butyl (5-(tetrahydro-2H-pyran-4-yl)thiazolo[5,4-b]pyridin-2-yl)carbamate

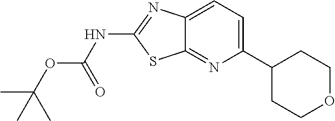 [SMILES: CC(C)(C)OC(=O)Nc1nc2ccc(C3CCOCC3)nc2s1]

To a mixture of tert-butyl (5-(3,6-dihydro-2H-pyran-4-yl)thiazolo[5,4-b]pyridin-2-yl)carbamate (200 mg, 0.6 mmol) in EtOH (60 mL) was added 10 wt. % Pd/C (360 mg). The mixture was stirred under 1 atm hydrogen at room temperature for 18 h. The mixture was filtered over celite and the filtrate was concentrated under reduced pressure. The residue was purified over silica gel by combi-flash, eluting with methanol in dichloromethane (5%, v/v) to give the title compound (125 mg, 62%). LC-MS (M+H)⁺=336.2.

### Step 3: 5-(tetrahydro-2H-pyran-4-yl)thiazolo[5,4-b]pyridin-2-amine

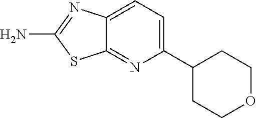 [SMILES: Nc1nc2ccc(C3CCOCC3)nc2s1]

The title compound (110 mg, crude) was prepared in a manner similar to that in Example 1 step 6 from tert-butyl (5-(tetrahydro-2H-pyran-4-yl)thiazolo[5,4-b]pyridin-2-yl)carbamate. LC-MS (M+H)⁺=236.1.

### Step 4: 4-(5-cyano-2-methoxyphenyl)-6-methyl-N-(5-(tetrahydro-2H-pyran-4-yl)thiazolo[5,4-b]pyridin-2-yl)nicotinamide

The title compound (32 mg, 38%) was prepared in a manner similar to that in Example 25 step 3 from 4-(5-cyano-2-methoxyphenyl)-6-methylnicotinic acid (55 mg, 0.2 mmol) and 5-(tetrahydro-2H-pyran-4-yl)thiazolo[5,4-b]pyridin-2-amine. ¹H-NMR (400 MHz, d6-DMSO) δ:12.86 (s, 1H), 8.78 (s, 1H), 8.04 (d, J=8.2 Hz, 1H), 7.93-7.85 (m, 2H), 7.41 (d, J=8.9 Hz, 2H), 7.17 (d, J=8.5 Hz, 1H), 3.99-3.55 (m, 2H), 3.58 (s, 3H), 3.52-3.41 (m, 2H), 3.09-3.01 (m, 1H), 2.59 (s, 3H), 1.83-1.77 (m, 4H). LC-MS (M+H)⁺=486.1.

### Example 68: 4-(benzo[d][1,3]dioxol-4-yl)-N-(5-(4-cyanophenyl)thiazolo[5,4-b]pyridin-2-yl)-6-methylnicotinamide

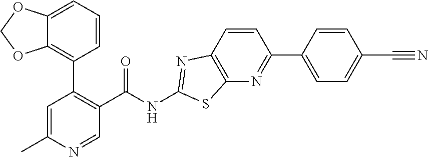 [SMILES: Cc1cc(-c2cccc3c2OCO3)c(C(=O)Nc2nc3ccc(-c4ccc(C#N)cc4)nc3s2)cn1]

### Step 1: 2-(benzo[d][1,3]dioxol-4-yl)-4,4,5,5-tetramethyl-1,3,2-dioxaborolane

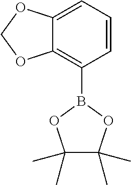 [SMILES: CC1(C)OB(c2cccc3c2OCO3)OC1(C)C]

The title compound (410 mg, 83%) was prepared in a manner similar to that in Example 28 step 2 from 4-bromobenzo[d][1,3]dioxole. LC-MS (M+H)⁺=249.1.

### Step 2: methyl 4-(benzo[d][1,3]dioxol-4-yl)-6-methylnicotinate

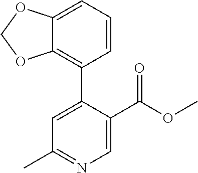 [SMILES: COC(=O)c1cnc(C)cc1-c1cccc2c1OCO2]

The title compound (352 mg, 81%) was prepared in a manner similar to that in Example 2 step 2 from 2-(benzo[d][1,3]dioxol-4-yl)-4,4,5,5-tetramethyl-1,3,2-dioxaborolane and methyl 4-chloro-6-methylnicotinate. LC-MS (M+H)⁺=272.1.

### Step 3: 4-(benzo[d][1,3]dioxol-4-yl)-6-methylnicotinic acid

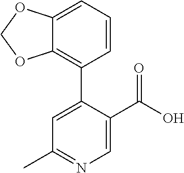 [SMILES: Cc1cc(-c2cccc3c2OCO3)c(C(=O)O)cn1]

The title compound (206 mg, 90%) was prepared in a manner similar to that in Example 1 step 3 from methyl 4-(benzo[d][1,3]dioxol-4-yl)-6-methylnicotinate. LC-MS (M+H)⁺=258.1.

### Step 4: 4-(benzo[d][1,3]dioxol-4-yl)-N-(5-(4-cyanophenyl)thiazolo[5,4-b]pyridin-2-yl)-6-methylnicotinamide

The title compound (2.5 mg, 2.5%) was prepared in a manner similar to that in Example 11 step 3 from 4-(2-aminothiazolo[5,4-b]pyridin-5-yl)benzonitrile and 4-(benzo[d][1,3]dioxol-4-yl)-6-methylnicotinic acid. ¹H-NMR (400 MHz, d6-DMSO) δ: 13.16 (s, 1H), 8.82 (s, 1H), 8.40-8.32 (m, 2H), 8.26-8.21 (m, 2H), 8.02-7.95 (m, 2H), 7.50 (s, 1H), 7.03-6.94 (m, 3H), 5.84 (s, 2H), 2.61 (s, 3H). LC-MS (M+H)⁺=492.1.

### Example 69: N-(5-(4-chlorobenzyl)thiazolo[5,4-b]pyridin-2-yl)-5-(2-methoxyphenyl)pyridazine-4-carboxamide

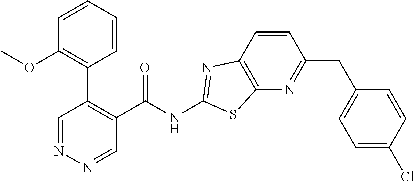 [SMILES: COc1ccccc1-c1cnncc1C(=O)Nc1nc2ccc(Cc3ccc(Cl)cc3)nc2s1]

### Step 1: 5-(4-chlorobenzyl)thiazolo[5,4-b]pyridin-2-amine

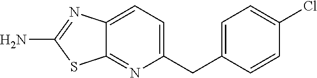 [SMILES: Nc1nc2ccc(Cc3ccc(Cl)cc3)nc2s1]

To a mixture of Zn (825 mg, 12.627 mmol) in THF (5 mL) was added Me₃SiCl (7 uL, 0.066 mmol) dropwise at room temperature under nitrogen atmosphere. To the above mixture was added 1-(bromomethyl)-4-chlorobenzene (1.22 g, 5.905 mmol) in THF (5 mL) dropwise over 5 min at room temperature under nitrogen atmosphere. The resulting mixture was stirred for 10 min at room temperature under nitrogen atmosphere. The mixture was added to a solution of tert-butyl (5-bromothiazolo[5,4-b]pyridin-2-yl)carbamate (500 mg, 1.514 mmol) in THF (5 mL) at room temperature. The resulting mixture was stirred at room temperature for 1 h under nitrogen atmosphere before concentrated. The residue was purified over silica gel by combi-flash, eluting with ethyl acetate in petroleum ether (0% to 25%, v/v) to give the title compound (100 mg, 23%). LC-MS (M+H)⁺=276.0.

### Step 2: 2-bromo-5-(4-chlorobenzyl)thiazolo[5,4-b]pyridine

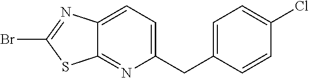 [SMILES: Clc1ccc(Cc2ccc3nc(Br)sc3n2)cc1]

To a solution of 5-(4-chlorobenzyl)thiazolo[5,4-b]pyridin-2-amine (100 mg, 0.362 mmol) and t-BuNO₂ (50 mg, 0.435 mmol) in MeCN (6 ml) were added TsOH (75 mg, 0.435 mmol) and CuBr₂ (1 mg, 0.003 mmol) at room temperature under nitrogen atmosphere. The resulting mixture was stirred at room temperature for 3 h under nitrogen atmosphere before concentration. The residue was purified over silica gel by combi-flash, eluting with ethyl acetate in petroleum ether (0% to 10%, v/v) to give the title compound (60 mg, 48%). LC-MS (M+H)⁺=388.8.

### Step 3: 5-(2-methoxyphenyl)pyridazine-4-carboxamide

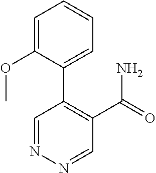 [SMILES: COc1ccccc1-c1cnncc1C(N)=O]

To a mixture of 5-(2-methoxyphenyl)pyridazine-4-carboxylic acid (291 mg, 1.269 mmol) and HATU (724 mg, 1.903 mmol) in N,N-dimethylformamide (5 mL) was added N,N-diisopropylethylamine (492 mg, 3.807 mmol) at room temperature. The resulting mixture was stirred at room temperature for 30 min under nitrogen atmosphere. To the above mixture was added NH₄Cl (102 mg, 1.903 mmol) at room temperature. The resulting mixture was stirred for additional 16 h at room temperature before concentration. The residue was purified over silica gel by combi-flash, eluting with methanol in dichloromethane (0% to 5%, v/v) to give the title compound (190 mg, 65%). LC-MS (M+H)⁺=230.2.

### Step 4: N-(5-(4-chlorobenzyl)thiazolo[5,4-b]pyridin-2-yl)-5-(2-methoxyphenyl)pyridazine-4-carboxamide

To a solution of 2-bromo-5-(4-chlorobenzyl)thiazolo[5,4-b]pyridine (30 mg, 0.088 mmol) and 5-(2-methoxyphenyl)pyridazine-4-carboxamide (20 mg, 0.087 mmol) in dioxane (4 mL) were added Pd₂(dba)₃ (8 mg, 0.009 mmol), XantPhos (16 mg, 0.026 mmol), and Cs₂CO₃ (60 mg, 0.174 mmol) at room temperature under nitrogen atmosphere. The resulting mixture was stirred at 100° C. for 16 h under nitrogen atmosphere before cooled to room temperature and concentrated. The residue was purified by prep-HPLC under the following conditions: column, XBridge Shield RP18 OBD Column, 30×150 mm, 5 um; mobile phase, acetonitrile in water (with 10 mmol/L NH₄HCO₃ and 0.1% NH₄OH), 20% to 50% gradient in 8 min; detector, UV 254 nm. The title compound (8 mg, 19%) was obtained. 1H NMR (400 MHz, d6-DMSO) δ 13.19 (brs, 1H), 9.47 (s, 1H), 9.41 (s, 1H), 8.07 (d, J=8.3 Hz, 1H), 7.56-7.30 (m, 7H), 7.18-7.10 (m, 1H), 7.08-7.01 (m, 1H), 4.20 (s, 2H), 3.34 (s, 3H). LC-MS (M+H)⁺=488.0.

### Example 70: N-(5-(4-cyanophenyl)thiazolo[5,4-b]pyridin-2-yl)-6-methyl-4-(3-oxo-3,4-dihydro-2H-benzo[b][1,4]oxazin-8-yl)nicotinamide

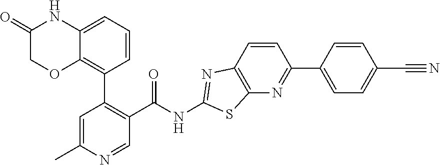 [SMILES: Cc1cc(-c2cccc3c2OCC(=O)N3)c(C(=O)Nc2nc3ccc(-c4ccc(C#N)cc4)nc3s2)cn1]

### Step 1: 8-bromo-2H-benzo[b][1,4]oxazin-3(4H)-one

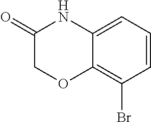 [SMILES: O=C1COc2c(Br)cccc2N1]

To a solution of 2-amino-6-bromophenol (2.0 g, 10.7 mmol) in acetonitrile (25 mL) were added K₂CO₃ (3.0 g, 21.4 mmol) and 3-chloropropanoyl chloride (2.0 g, 16.1 mmol). The reaction mixture was stirred at 80° C. for 14 h before cooled to room temperature and diluted with ethyl acetate (50 mL). The mixture was washed with brine (30 mL), dried over sodium sulfate, filtered and concentrated under reduce pressure to give title compound (1.5 g, 62%) which was used to the next step without further purification. LC-MS (M+H)⁺=227.8.

### Step 2: 8-(4,4,5,5-tetramethyl-1,3,2-dioxaborolan-2-yl)-2H-benzo[b][1,4]oxazin-3(4H)-one

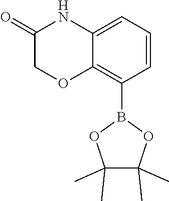 [SMILES: CC1(C)OB(c2cccc3c2OCC(=O)N3)OC1(C)C]

The title compound (820 mg, 64%) was prepared in a manner similar to that in Example 28 step 2 from 8-bromo-2H-benzo[b][1,4]oxazin-3(4H)-one. LC-MS (M+H)⁺=276.1.

### Step 3: methyl 6-methyl-4-(3-oxo-3,4-dihydro-2H-benzo[b][1,4]oxazin-8-yl)nicotinate

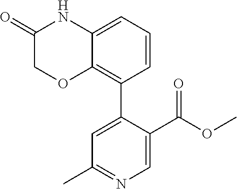 [SMILES: COC(=O)c1cnc(C)cc1-c1cccc2c1OCC(=O)N2]

The title compound (360 mg, 66%) was prepared in a manner similar to that described in Example 2 step 2 from 8-(4,4,5,5-tetramethyl-1,3,2-dioxaborolan-2-yl)-2H-benzo[b][1,4]oxazin-3(4H)-one and methyl 4-chloro-6-methylnicotinate. LC-MS (M+H)⁺=299.1.

### Step 4: 6-methyl-4-(3-oxo-3,4-dihydro-2H-benzo[b][1,4]oxazin-8-yl)nicotinic acid

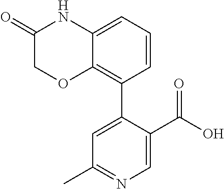 [SMILES: Cc1cc(-c2cccc3c2OCC(=O)N3)c(C(=O)O)cn1]

The title compound (160 mg, 83%) was prepared in a manner similar to that described in Example 1 step 3 from methyl 6-methyl-4-(3-oxo-3,4-dihydro-2H-benzo[b][1,4]oxazin-8-yl)nicotinate. LC-MS (M+H)⁺=285.1.

### Step 5: N-(5-(4-cyanophenyl)thiazolo[5,4-b]pyridin-2-yl)-6-methyl-4-(3-oxo-3,4-dihydro-2H-benzo[b][1,4]oxazin-8-yl)nicotinamide

The title compound (15 mg, 14%) was prepared in a manner similar to that described in Example 25 step 3 from 4-(2-aminothiazolo[5,4-b]pyridin-5-yl)benzonitrile and 6-methyl-4-(3-oxo-3,4-dihydro-2H-benzo[b][1,4]oxazin-8-yl)nicotinic acid. ¹H-NMR (400 MHz, d6-DMSO) δ:13.18 (s, 1H), 10.80 (s, 1H), 8.81 (s, 1H), 8.38-8.30 (m, 2H), 8.29-8.19 (m, 2H), 8.01-7.92 (m, 2H), 7.38 (s, 1H), 7.15-7.01 (m, 2H), 7.00-6.95 (m, 1H), 4.25 (s, 2H), 2.60 (s, 3H). LC-MS (M+H)⁺=519.1.

### Example 71: N-(5-(4-(cyclopropanecarboxamido)phenyl)thiazolo[5,4-b]pyridin-2-yl)-5-(2-methoxyphenyl)pyridazine-4-carboxamide

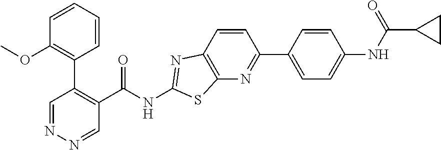 [SMILES: COc1ccccc1-c1cnncc1C(=O)Nc1nc2ccc(-c3ccc(NC(=O)C4CC4)cc3)nc2s1]

### Step 1: N-(4-(4,4,5,5-tetramethyl-1,3,2-dioxaborolan-2-yl)phenyl)cyclopropanecarboxamide

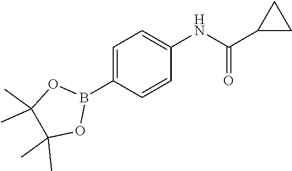 [SMILES: CC1(C)OB(c2ccc(NC(=O)C3CC3)cc2)OC1(C)C]

To a solution of 4-(4,4,5,5-tetramethyl-1,3,2-dioxaborolan-2-yl)aniline (910 mg, 4.150 mmol) in dichloromethane (15 mL) were added triethylamine (630 mg, 6.225 mmol) and cyclopropanecarbonyl chloride (475 mg, 4.565 mmol) at 0° C. under nitrogen atmosphere. The resulting mixture was stirred at room temperature for 4 h under nitrogen atmosphere before concentration. The residue was treated with water (50 ml) and the resulting mixture was extracted with ethyl acetate (2×50 mL). The combined organic layers were washed with brine, dried over Na₂SO₄, filtered and concentrated. The residue was purified over silica gel by combi-flash, eluting with ethyl acetate in petroleum ether (0% to 25%, v/v) to give the title compound (768 mg, 64%). LC-MS (M+H)⁺=287.9.

### Step 2: N-(4-(2-aminothiazolo[5,4-b]pyridin-5-yl)phenyl)cyclopropanecarboxamide

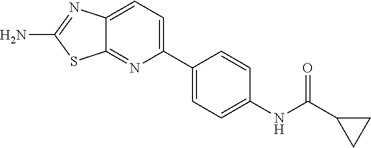 [SMILES: Nc1nc2ccc(-c3ccc(NC(=O)C4CC4)cc3)nc2s1]

The title compound (142 mg, 51%) was prepared in a manner similar to that in Example 1 step 5 from N-(4-(4,4,5,5-tetramethyl-1,3,2-dioxaborolan-2-yl)phenyl)cyclopropanecarboxamide and 5-bromothiazolo[5,4-b]pyridin-2-amine. LC-MS (M+H)⁺=311.0.

### Step 3: N-(5-(4-(cyclopropanecarboxamido)phenyl)thiazolo[5,4-b]pyridin-2-yl)-5-(2-methoxyphenyl)pyridazine-4-carboxamide

The title compound (12 mg, 21%) was prepared in a manner similar to that in Example 4 step 2 from N-(4-(2-aminothiazolo[5,4-b]pyridin-5-yl)phenyl)cyclopropanecarboxamide and 5-(2-methoxyphenyl)pyridazine-4-carboxylic acid. 1H NMR (300 MHz, Methanol-d4) δ 13.27 (brs, 1H), 10.66 (s, 1H), 9.50 (s, 1H), 9.43 (s, 1H), 8.12-7.97 (m, 2H), 7.93 (d, J=8.6 Hz, 1H), 7.71 (d, J=8.6 Hz, 2H), 7.58-7.44 (m, 2H), 7.23-7.02 (m, 2H), 3.65 (s, 3H), 1.93-1.65 (m, 1H), 1.03-0.83 (m, 4H). LC-MS (M+H)⁺=523.0.

### Example 72: N-(5-(4-cyanophenyl)thiazolo[5,4-b]pyridin-2-yl)-4-(2-methoxyphenyl)-6-methylnicotinamide

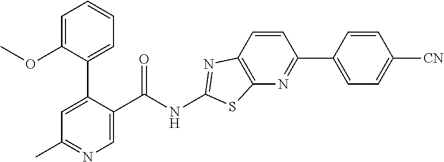 [SMILES: COc1ccccc1-c1cc(C)ncc1C(=O)Nc1nc2ccc(-c3ccc(C#N)cc3)nc2s1]

The title compound (25 mg, 8%) was prepared in a manner similar to that described in Example 25 step 3 from 4-(2-methoxyphenyl)-6-methylnicotinic acid and 4-(2-aminothiazolo[5,4-b]pyridin-5-yl)benzonitrile. ¹H-NMR (400 MHz, d6-DMSO) δ 12.98 (s, 1H), 8.75 (s, 1H), 8.34 (d, J=8.4 Hz, 2H), 8.26-8.14 (m, 2H), 7.97 (d, J=8.4 Hz, 2H), 7.45-7.36 (m, 2H), 7.33 (s, 1H), 7.09 (t, J=7.4 Hz, 1H), 6.98 (d, J=8.6 Hz, 1H), 3.52 (s, 3H), 2.59 (s, 3H). LC-MS (M+H)⁺=478.6.

### Example 73: 4-(5-cyano-2-methoxyphenyl)-N-(5-(4-(ethylcarbamoyl)phenyl)thiazolo[5,4-b]pyridin-2-yl)-6-methylnicotinamide

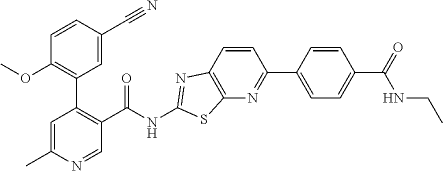 [SMILES: CCNC(=O)c1ccc(-c2ccc3nc(NC(=O)c4cnc(C)cc4-c4cc(C#N)ccc4OC)sc3n2)cc1]

### Step 1: methyl 4-(2-((tert-butoxycarbonyl)amino)thiazolo[5,4-b]pyridin-5-yl)benzoate

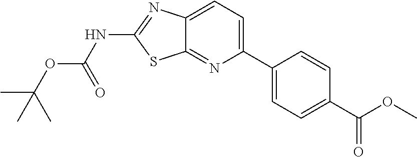 [SMILES: COC(=O)c1ccc(-c2ccc3nc(NC(=O)OC(C)(C)C)sc3n2)cc1]

The title compound (4.2 g, 74%) was prepared in a manner similar to that in Example 1 step 5 from tert-butyl (5-bromothiazolo[5,4-b]pyridin-2-yl)carbamate and (4-(methoxycarbonyl)phenyl)boronic acid. LC-MS (M+H)⁺=386.4.

### Step 2: methyl 4-(2-aminothiazolo[5,4-b]pyridin-5-yl)benzoate

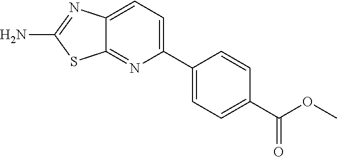 [SMILES: COC(=O)c1ccc(-c2ccc3nc(N)sc3n2)cc1]

The title compound (2.1 g, 68%) was prepared in a manner similar to that in Example 1 step 6 from methyl 4-(2-((tert-butoxycarbonyl)amino)thiazolo[5,4-b]pyridin-5-yl)benzoate. LC-MS (M+H)⁺=286.6.

### Step 3: methyl 4-(2-(4-(5-cyano-2-methoxyphenyl)-6-methylnicotinamido)thiazolo[5,4-b]pyridin-5-yl)benzoate

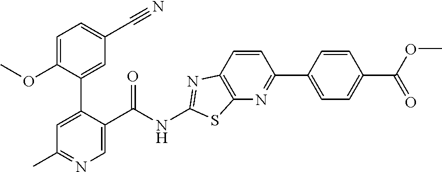

The title compound (1.7 g, 92%) was prepared in a manner similar to that in Example 11 step 3 from 4-(5-cyano-2-methoxyphenyl)-6-methylnicotinic acid and methyl 4-(2-aminothiazolo[5,4-b]pyridin-5-yl)benzoate. LC-MS (M+H)⁺=536.2.

### Step 4: 4-(2-(4-(5-cyano-2-methoxyphenyl)-6-methylnicotinamido)thiazolo[5,4-b]pyridin-5-yl)benzoic acid

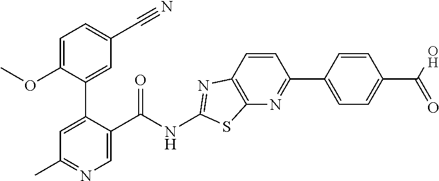 [SMILES: COc1ccc(C#N)cc1-c1cc(C)ncc1C(=O)Nc1nc2ccc(-c3ccc(C(=O)O)cc3)nc2s1]

The title compound (1.6 g, 97%) was prepared in a manner similar to that in Example 1 step 3 from methyl 4-(2-(4-(5-cyano-2-methoxyphenyl)-6-methylnicotinamido)thiazolo[5,4-b]pyridin-5-yl)benzoate. LC-MS (M+H)⁺=522.3.

### Step 5: 4-(5-cyano-2-methoxyphenyl)-N-(5-(4-(ethylcarbamoyl)phenyl)thiazolo[5,4-b]pyridin-2-yl)-6-methylnicotinamide

To a solution of 4-(2-(4-(5-cyano-2-methoxyphenyl)-6-methylnicotinamido)thiazolo[5,4-b]pyridin-5-yl)benzoic acid (100 mg, 0.2 mmol) in N,N-dimethylformamide (5 mL) were added 1-[bis(dimethylamino)methylene]-1H-1,2,3-triazolo[4,5-b]pyridinium 3-oxid hexafluorophosphate (0.11 g, 0.3 mmol) and N,N-diisopropylethylamine (52 mg, 0.4 mmol). The reaction mixture was stirred for 10 min before addition of 30% EtNH₂ in EtOH (18 mg, 0.4 mmol). The resulting mixture was stirred at room temperature for 14 h. The mixture was purified by prep-HPLC (Elution: acetonitrile (0.1% formic acid)/water (0.1% formic acid): 50%-80%, v/v) to give the title compound (50 mg, 46%). ¹H-NMR (400 MHz, d6-DMSO) δ 13.01 (s, 1H), 8.81 (s, 1H), 8.56 (t, J=5.5 Hz, 1H), 8.23-8.13 (m, 4H), 8.03-7.86 (m, 4H), 7.44 (s, 1H), 7.18 (d, J=9.3 Hz, 1H), 3.60 (s, 3H), 3.35-3.28 (m, 2H), 2.61 (s, 3H), 1.15 (t, J=7.2 Hz, 3H). LC-MS (M+H)⁺=549.4.

### Example 74: 4-(5-cyano-2-methoxyphenyl)-N-(5-(4-(isopropylcarbamoyl)phenyl)thiazolo[5,4-b]pyridin-2-yl)-6-methylnicotinamide

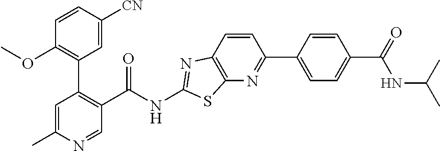 [SMILES: COc1ccc(C#N)cc1-c1cc(C)ncc1C(=O)Nc1nc2ccc(-c3ccc(C(=O)NC(C)C)cc3)nc2s1]

The title compound (1 mg, 9%) was prepared in a manner similar to Example 73 of Step 5 from 4-(2-(4-(5-cyano-2-methoxyphenyl)-6-methylnicotinamido)thiazolo[5,4-b]pyridin-5-yl)benzoic acid and isopropylamine. ¹H-NMR (400 MHz, d6-DMSO) δ 12.99 (s, 1H), 8.85 (s, 1H), 8.30 (d, J=8.3 Hz, 1H), 8.21-8.12 (m, 4H), 7.97 (d, J=8.4 Hz, 2H), 7.93-7.82 (m, 2H), 7.39 (s, 1H), 7.18 (d, J=8.7 Hz, 1H), 4.17-4.09 (m, 1H), 3.60 (s, 3H), 2.59 (s, 3H), 1.19 (d, J=6.6 Hz, 6H). LC-MS (M+H)⁺=563.3.

### Example 75: N-(5-(4-cyanophenyl)thiazolo[5,4-b]pyridin-2-yl)-4-(3-fluoro-2-methoxyphenyl)-6-methylnicotinamide

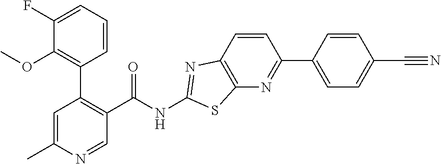 [SMILES: COc1c(F)cccc1-c1cc(C)ncc1C(=O)Nc1nc2ccc(-c3ccc(C#N)cc3)nc2s1]

### Step 1: methyl 4-(3-fluoro-2-methoxyphenyl)-6-methylnicotinate

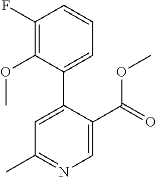 [SMILES: COC(=O)c1cnc(C)cc1-c1cccc(F)c1OC]

The title compound (400 mg, 90%) was prepared in a manner similar to that in Example 2 step 2 from methyl 4-chloro-6-methylnicotinate and (3-fluoro-2-methoxyphenyl)boronic acid. LC-MS (M+H)⁺=276.1.

### Step 2: 4-(3-fluoro-2-methoxyphenyl)-6-methylnicotinic acid

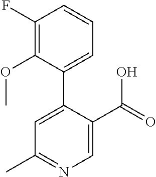 [SMILES: COc1c(F)cccc1-c1cc(C)ncc1C(=O)O]

The title compound (300 mg, 90%) was prepared in a manner similar to that in Example 1 step 3 from methyl 4-(3-fluoro-2-methoxyphenyl)-6-methylnicotinate. LC-MS (M+H)⁺=262.0.

### Step 3: 4-(3-fluoro-2-methoxyphenyl)-6-methylnicotinamide

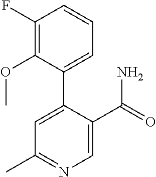 [SMILES: COc1c(F)cccc1-c1cc(C)ncc1C(N)=O]

The title compound (87 mg, 88%) was prepared in a manner similar to that in Example 69 step 3 from 4-(3-fluoro-2-methoxyphenyl)-6-methylnicotinic acid and NH₄Cl. LC-MS (M+H)⁺=261.0.

### Step 4: 4-(2-bromothiazolo[5,4-b]pyridin-5-yl)benzonitrile

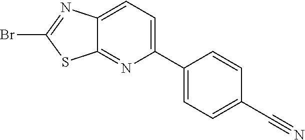 [SMILES: N#Cc1ccc(-c2ccc3nc(Br)sc3n2)cc1]

The title compound (65 mg, 59%) was prepared in a manner similar to that in Example 69 step 2 from 4-(2-aminothiazolo[5,4-b]pyridin-5-yl)benzonitrile. LC-MS (M+H)⁺=316.1.

### Step 5: N-(5-(4-cyanophenyl)thiazolo[5,4-b]pyridin-2-yl)-4-(3-fluoro-2-methoxyphenyl)-6-methylnicotinamide

The title compound (10 mg, 27%) was prepared in a manner similar to that in Example 69 step 4 from 4-(2-bromothiazolo[5,4-b]pyridin-5-yl)benzonitrile and 4-(3-fluoro-2-methoxyphenyl)-6-methylnicotinamide. ¹H NMR (400 MHz, d6-DMSO) δ 13.19 (brs, 1H), 8.86 (s, 1H), 8.37-8.18 (m, 4H), 8.00-7.93 (m, 2H), 7.46-7.29 (m, 2H), 7.27-7.17 (m, 2H), 3.63-3.58 (m, 3H), 2.61 (s, 3H). LC-MS (M+H)⁺=496.1.

### Example 76: N-(5-(4-cyanophenyl)thiazolo[5,4-b]pyridin-2-yl)-4-(2-methoxy-5-nitrophenyl)-6-methylnicotinamide

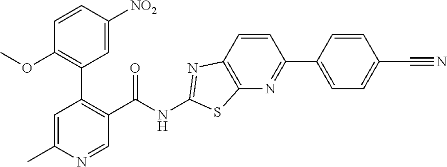 [SMILES: COc1ccc([N+](=O)[O-])cc1-c1cc(C)ncc1C(=O)Nc1nc2ccc(-c3ccc(C#N)cc3)nc2s1]

### Step 1: methyl 4-(2-methoxy-5-nitrophenyl)-6-methylnicotinate

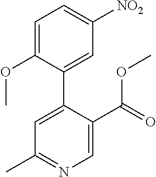 [SMILES: COC(=O)c1cnc(C)cc1-c1cc([N+](=O)[O-])ccc1OC]

The title compound (260 mg, 66%) was prepared in a manner similar to that described in Example 2 step 2 from (2-methoxy-5-nitrophenyl)boronic acid and methyl 4-chloro-6-methylnicotinate. LC-MS (M+H)⁺=303.1.

### Step 2: 4-(2-methoxy-5-nitrophenyl)-6-methylnicotinic acid

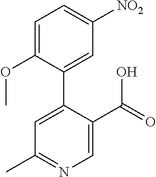 [SMILES: COc1ccc([N+](=O)[O-])cc1-c1cc(C)ncc1C(=O)O]

The title compound (200 mg, 83%) was prepared in a manner similar to that described in Example 1 step 3 from methyl 4-(2-methoxy-5-nitrophenyl)-6-methylnicotinate. LC-MS (M+H)⁺=289.1.

### Step 3: N-(5-(4-cyanophenyl)thiazolo[5,4-b]pyridin-2-yl)-4-(2-methoxy-5-nitrophenyl)-6-methylnicotinamide

The title compound (50 mg, 39%) was prepared in a manner similar to that described in Example 25 step 3 from 4-(2-methoxy-5-nitrophenyl)-6-methylnicotinic acid and 4-(2-aminothiazolo[5,4-b]pyridin-5-yl)benzonitrile. ¹H-NMR (400 MHz, d6-DMSO) δ 13.09 (s, 1H), 8.84 (s, 1H), 8.38-8.31 (m, 3H), 8.29 (d, J=2.8 Hz, 1H), 8.27-8.20 (m, 2H), 7.97 (d, J=8.3 Hz, 2H), 7.50 (s, 1H), 7.24 (d, J=9.1 Hz, 1H), 3.67 (s, 3H), 2.63 (s, 3H). LC-MS (M+H)⁺=523.5.

### Example 77: 4-(5-cyano-2-methoxyphenyl)-N-(5-(2-methoxyethoxy)thiazolo[5,4-b]pyridin-2-yl)-6-methylnicotinamide

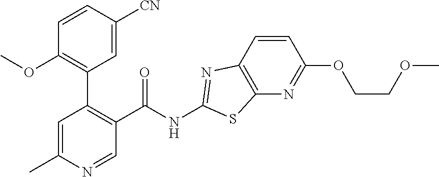

### Step 1: tert-butyl (5-(2-methoxyethoxy)thiazolo[5,4-b]pyridin-2-yl)carbamate

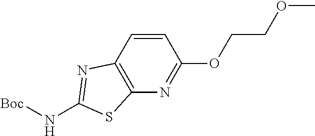 [SMILES: COCCOc1ccc2nc(NC(=O)OC(C)(C)C)sc2n1]

The title compound (150 mg, 46%) was prepared in a manner similar to that in Example 65 step 1 from 2-methoxyethan-1-ol and tert-butyl (5-bromothiazolo[5,4-b]pyridin-2-yl)carbamate. LC-MS (M+H)⁺=326.1.

### Step 2: 5-(2-methoxyethoxy)thiazolo[5,4-b]pyridin-2-amine

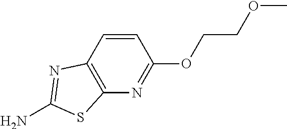

The title compound (48 mg, 46%) was prepared in a manner similar to that in Example 1 step 6 from tert-butyl (5-(2-methoxyethoxy)thiazolo[5,4-b]pyridin-2-yl)carbamate. LC-MS (M+H)⁺=226.1.

### Step 3: 4-(5-cyano-2-methoxyphenyl)-N-(5-(2-methoxyethoxy)thiazolo[5,4-b]pyridin-2-yl)-6-methylnicotinamide

The title compound (6 mg, 6%) was prepared in a manner similar to that in Example 25 step 3 from 4-(5-cyano-2-methoxyphenyl)-6-methylnicotinic acid and 5-(2-methoxyethoxy)thiazolo[5,4-b]pyridin-2-amine. ¹H-NMR (400 MHz, d6-DMSO) δ 12.77 (s, 1H), 8.78 (s, 1H), 8.03 (d, J=8.7 Hz, 1H), 7.91-7.86 (m, 2H), 7.40 (s, 1H), 7.17 (d, J=8.7 Hz, 1H), 6.91 (d, J=8.8 Hz, 1H), 4.51-4.30 (m, 2H), 3.74-3.63 (m, 2H), 3.58 (s, 3H), 3.31 (s, 3H), 2.59 (s, 3H). LC-MS (M+H)⁺=476.5.

### Example 78: 4-(5-cyano-2-methoxyphenyl)-6-methyl-N-(5-(3-methyl-4-(2-oxopyrrolidin-1-yl)phenyl)thiazolo[5,4-b]pyridin-2-yl)nicotinamide

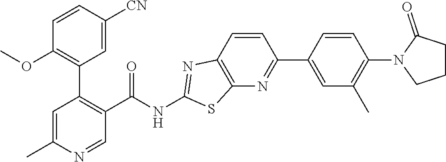 [SMILES: COc1ccc(C#N)cc1-c1cc(C)ncc1C(=O)Nc1nc2ccc(-c3ccc(N4CCCC4=O)c(C)c3)nc2s1]

### Step 1: 1-(4-bromo-2-methylphenyl)pyrrolidin-2-one

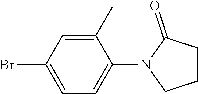

The solution of 4-bromo-2-methylaniline (0.37 g, 2 mmol), Et₃N (0.4 g, 4 mmol) in dichloromethane (10 mL) was added 4-chlorobutanoyl chloride (0.42 g, 3 mmol) carefully. It was stirred at room temperature for 14 h. The mixture was diluted with dichloromethane and washed by brine, the organic phase was dried and evaporated. The residue was used directly without purification. It was dissolved in N,N-dimethylformamide (3 mL) and NaH (0.16 g, 4 mmol) was added. The mixture was stirred for 2 hours. Some trash ice added and extracted with ethyl acetate, washed by brine, dried and evaporated. The crude product was purified by column chromatography, eluting with ethyl acetate in petroleum ether (50%, v/v) to give the title compound (0.48 g, 96%). LC-MS (M+H)⁺=254.2.

### Step 2: 1-(2-methyl-4-(4,4,5,5-tetramethyl-1,3,2-dioxaborolan-2-yl)phenyl)pyrrolidin-2-one

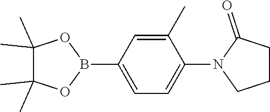 [SMILES: Cc1cc(B2OC(C)(C)C(C)(C)O2)ccc1N1CCCC1=O]

The title compound (0.34 g, 59%) was prepared in a manner similar to that in Example 28 step 2 from 1-(4-bromo-2-methylphenyl)pyrrolidin-2-one and bis(pinacolato)diboron. LC-MS (M+H)⁺=302.4.

### Step 3: tert-butyl (5-(3-methyl-4-(2-oxopyrrolidin-1-yl)phenyl)thiazolo[5,4-b]pyridin-2-yl)carbamate

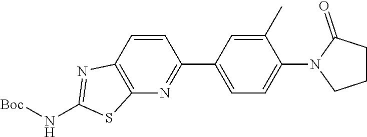 [SMILES: Cc1cc(-c2ccc3nc(NC(=O)OC(C)(C)C)sc3n2)ccc1N1CCCC1=O]

The reaction mixture of tert-butyl (5-bromothiazolo[5,4-b]pyridin-2-yl)carbamate (0.16 g, 0.5 mmol), 1-(2-methyl-4-(4,4,5,5-tetramethyl-1,3,2-dioxaborolan-2-yl)phenyl)pyrrolidin-2-one (0.18 g, 0.6 mmol), 1,1′-bis(diphenylphosphino)ferrocene]dichloropalladium(II) (41 mg, 0.05 mmol) and K₂CO₃ (0.14 g, 1 mmol) in dioxane/water (10 mL, 10:1 v/v) under nitrogen was stirred at 100° C. for 14 h. The mixture was filtered and the filtrate was evaporated. The residue was purified by column chromatography, eluting with ethyl acetate in petroleum ether (50%, v/v) to give the title compound (85 mg, 40%). LC-MS (M+H)⁺=425.3.

### Step 4: 1-(4-(2-aminothiazolo[5,4-b]pyridin-5-yl)-2-methylphenyl)pyrrolidin-2-one

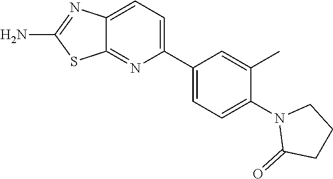 [SMILES: Cc1cc(-c2ccc3nc(N)sc3n2)ccc1N1CCCC1=O]

The title compound (45 mg, 69%) was prepared in a manner similar to that in Example 1 step 6 from tert-butyl (5-(3-methyl-4-(2-oxopyrrolidin-1-yl)phenyl)thiazolo[5,4-b]pyridin-2-yl)carbamate. LC-MS (M+H)⁺=325.3.

### Step 5: 4-(5-cyano-2-methoxyphenyl)-6-methyl-N-(5-(3-methyl-4-(2-oxopyrrolidin-1-yl)phenyl)thiazolo[5,4-b]pyridin-2-yl)nicotinamide

The title compound (8 mg, 10%) was prepared in a manner similar to that in Example 25 step 3 from 4-(5-cyano-2-methoxyphenyl)-6-methylnicotinic acid and 1-(4-(2-aminothiazolo[5,4-b]pyridin-5-yl)-2-methylphenyl)pyrrolidin-2-one. ¹H NMR (400 MHz, d6-DMSO) δ 12.98 (s, 1H), 8.80 (s, 1H), 8.19 (d, J=8.4 Hz, 1H), 8.12-8.03 (m, 2H), 7.98 (d, J=8.2 Hz, 1H), 7.92 (d, J=7.0 Hz, 2H), 7.44 (s, 1H), 7.34 (d, J=8.3 Hz, 1H), 7.18 (d, J=9.4 Hz, 1H), 3.73 (t, J=6.9 Hz, 2H), 3.60 (s, 3H), 2.60 (s, 3H), 2.44 (t, J=7.9 Hz, 2H), 2.25 (s, 3H), 2.19-2.12 (m, 2H). LC-MS (M+H)⁺=575.4.

### Example 79: 4-(5-cyano-2-methoxyphenyl)-N-(5-(4-(methoxymethyl)phenyl)thiazolo[5,4-b]pyridin-2-yl)-6-methylnicotinamide

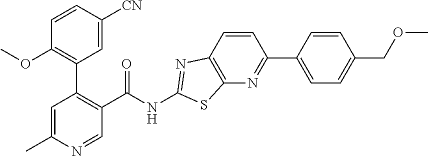

The title compound (18 mg, 28%) was prepared in a manner similar to that in Example 78 step 3 from N-(5-bromothiazolo[5,4-b]pyridin-2-yl)-4-(5-cyano-2-methoxyphenyl)-6-methylnicotinamide and (4-(methoxymethyl)phenyl)boronic acid. ¹H NMR (400 MHz, d6-DMSO) δ:12.97 (s, 1H), 8.81 (s, 1H), 8.21-8.04 (m, 4H), 7.94-7.87 (m, 2H), 7.48-7.42 (m, 3H), 7.18 (d, J=8.5 Hz, 1H), 4.48 (s, 2H), 3.60 (s, 3H), 3.33 (s, 3H), 2.60 (s, 3H). LC-MS (M+H)⁺=522.5.

### Example 80: 4-(5-cyano-2-methoxyphenyl)-N-(5-(4-(2-hydroxypropan-2-yl)phenyl)thiazolo[5,4-b]pyridin-2-yl)-6-methylnicotinamide

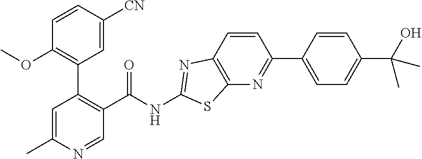 [SMILES: COc1ccc(C#N)cc1-c1cc(C)ncc1C(=O)Nc1nc2ccc(-c3ccc(C(C)(C)O)cc3)nc2s1]

The title compound (19 mg, 28%) was prepared in a manner similar to that in Example 78 step 3 from N-(5-bromothiazolo[5,4-b]pyridin-2-yl)-4-(5-cyano-2-methoxyphenyl)-6-methylnicotinamide and (4-(2-hydroxypropan-2-yl)phenyl)boronic acid. ¹H NMR (400 MHz, d6-DMSO) δ: 12.96 (s, 1H), 8.80 (s, 1H), 8.17 (d, J=8.5 Hz, 1H), 8.05 (d, J=8.4 Hz, 3H), 7.96-7.86 (m, 2H), 7.59 (d, J=8.4 Hz, 2H), 7.44 (s, 1H), 7.18 (d, J=9.2 Hz, 1H), 5.08 (s, 1H), 3.60 (s, 3H), 2.60 (s, 3H), 1.47 (s, 6H). LC-MS (M+H)⁺=536.3.

### Example 81: 5-(2-methoxyphenyl)-N-(5-(4-(methylsulfonyl)phenyl)thiazolo[5,4-b]pyridin-2-yl)pyridazine-4-carboxamide

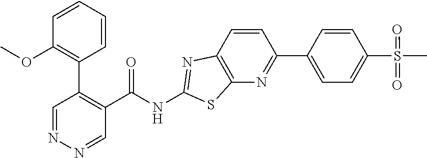

### Step 1: 5-(4-(methylsulfonyl)phenyl)thiazolo[5,4-b]pyridin-2-amine

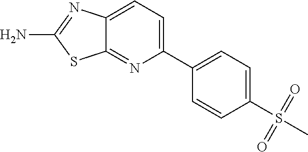 [SMILES: CS(=O)(=O)c1ccc(-c2ccc3nc(N)sc3n2)cc1]

The title compound (100 mg, 36%) was prepared in a manner similar to that in Example 78 step 3 from 5-bromothiazolo[5,4-b]pyridin-2-amine and (4-(methylsulfonyl)phenyl)boronic acid. LC-MS (M+H)⁺=306.0.

### Step 2: 5-(2-methoxyphenyl)-N-(5-(4-(methylsulfonyl)phenyl)thiazolo[5,4-b]pyridin-2-yl)pyridazine-4-carboxamide

To a solution of 5-(2-methoxyphenyl)pyridazine-4-carboxylic acid (39 mg, 0.170 mmol) and N,N-diisopropylethylamine (76 mg, 0.590 mmol) in N,N-dimethylformamide (1 mL) was added BTFFH (75 mg, 0.236 mmol) at room temperature under nitrogen atmosphere. The resulting mixture was stirred for 0.5 h at room temperature under nitrogen atmosphere. To the above mixture was added 5-(4-(methylsulfonyl)phenyl)thiazolo[5,4-b]pyridin-2-amine (40 mg, 0.131 mmol) at room temperature. The resulting mixture was stirred for additional 16 h at 80° C. After consumption of starting material, the resulting mixture concentrated under reduced pressure and the residue was purified by prep-HPLC under the following conditions: column, XBridge Shield RP18 OBD Column, 30×150 mm, 5 um; mobile phase, acetonitrile in water (with 10 mmol/L NH₄HCO₃ and 0.1% NH₄OH), 16% to 46% gradient in 8 min; detector, UV 254 nm. The title compound (20 mg, 29%) obtained. 1H NMR (400 MHz, d6-DMSO) δ 13.39 (brs, 1H), 9.52-9.47 (m, 1H), 9.38 (s, 1H), 8.44-8.36 (m, 2H), 8.19 (s, 2H), 8.07-8.02 (m, 2H), 7.55-7.43 (m, 2H), 7.17-7.03 (m, 2H), 3.57 (s, 3H), 3.28 (s, 3H). LC-MS (M+H)⁺=518.0.

### Example 82: 4-(3-cyano-2-methoxyphenyl)-N-(5-(4-cyanophenyl)thiazolo[5,4-b]pyridin-2-yl)-6-methylnicotinamide

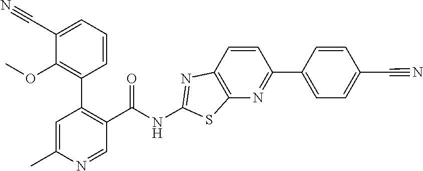 [SMILES: COc1c(C#N)cccc1-c1cc(C)ncc1C(=O)Nc1nc2ccc(-c3ccc(C#N)cc3)nc2s1]

### Step 1: methyl 4-(3-cyano-2-methoxyphenyl)-6-methylnicotinate

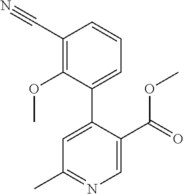 [SMILES: COC(=O)c1cnc(C)cc1-c1cccc(C#N)c1OC]

The title compound (77 mg, 54%) was prepared in a manner similar to that in Example 2 step 2 from 2-methoxy-3-(4,4,5,5-tetramethyl-1,3,2-dioxaborolan-2-yl)benzonitrile and methyl 4-chloro-6-methylnicotinate. LC-MS (M+H)⁺=283.1.

### Step 2: 4-(3-cyano-2-methoxyphenyl)-6-methylnicotinic acid

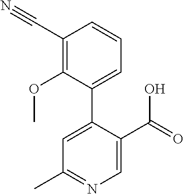 [SMILES: COc1c(C#N)cccc1-c1cc(C)ncc1C(=O)O]

The title compound (45 mg, 61%) was prepared in a manner similar to that in Example 1 step 3 from methyl 4-(3-cyano-2-methoxyphenyl)-6-methylnicotinate. LC-MS (M+H)⁺=269.2.

### Step 3: 4-(3-cyano-2-methoxyphenyl)-N-(5-(4-cyanophenyl)thiazolo[5,4-b]pyridin-2-yl)-6-methylnicotinamide

The title compound (11 mg, 16%) was prepared in a manner similar to that in Example 81 step 2 from 4-(3-cyano-2-methoxyphenyl)-6-methylnicotinic acid and 4-(2-aminothiazolo[5,4-b]pyridin-5-yl)benzonitrile. 1H NMR (300 MHz, d6-DMSO) δ 13.24 (brs, 1H), 8.92 (s, 1H), 8.39-8.17 (m, 4H), 8.01-7.93 (m, 2H), 7.92-7.82 (m, 1H), 7.77-7.68 (m, 1H), 7.47-7.36 (m, 2H), 3.61 (s, 3H), 2.62 (s, 3H). LC-MS (M+H)⁺=503.2.

### Example 83: N-(5-(4-cyanophenyl)thiazolo[5,4-b]pyridin-2-yl)-5-(2-methoxyphenyl)pyridazine-4-carboxamide

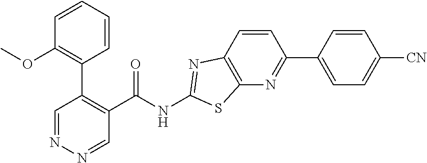 [SMILES: COc1ccccc1-c1cnncc1C(=O)Nc1nc2ccc(-c3ccc(C#N)cc3)nc2s1]

The title compound (15 mg, 7%) was prepared in a manner similar to that in Example 11 step 3 from 5-(2-methoxyphenyl)pyridazine-4-carboxylic acid and 4-(2-aminothiazolo[5,4-b]pyridin-5-yl)benzonitrile. 1H NMR (400 MHz, d6-DMSO) δ 13.35 (s, 1H), 9.50 (s, 1H), 9.43 (s, 1H), 8.35 (d, J=8.5 Hz, 2H), 8.31-8.21 (m, 2H), 7.98 (d, J=8.5 Hz, 2H), 7.54 (d, J=7.7 Hz, 1H), 7.52-7.46 (m, 1H), 7.19-7.12 (m, 1H), 7.07 (d, J=8.3 Hz, 1H), 3.56 (s, 3H). LC-MS (M+H)⁺=465.4.

### Example 84: 4-(5-cyano-2-methoxyphenyl)-6-methyl-N-(5-(4-(morpholine-4-carbonyl)phenyl)thiazolo[5,4-b]pyridin-2-yl)nicotinamide

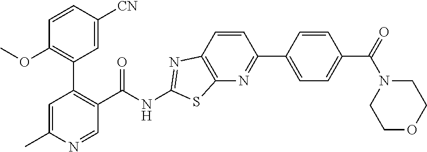 [SMILES: COc1ccc(C#N)cc1-c1cc(C)ncc1C(=O)Nc1nc2ccc(-c3ccc(C(=O)N4CCOCC4)cc3)nc2s1]

The title compound (60 mg, 50%) was prepared in a manner similar to Example 73 step 5 from 4-(2-(4-(5-cyano-2-methoxyphenyl)-6-methylnicotinamido)thiazolo[5,4-b]pyridin-5-yl)benzoic acid and morpholine. ¹H NMR (400 MHz, d6-DMSO) δ 13.02 (s, 1H), 8.81 (s, 1H), 8.23-8.12 (m, 4H), 7.98-7.88 (m, 2H), 7.55 (d, J=8.3 Hz, 2H), 7.45 (s, 1H), 7.19 (d, J=9.4 Hz, 1H), 3.69-3.55 (m, 8H), 3.37 (s, 3H), 2.61 (s, 3H). LC-MS (M+H)⁺=591.4.

### Example 85: 4-(5-cyano-2-methoxyphenyl)-6-methyl-N-(5-(4-(pyrrolidine-1-carbonyl)phenyl)thiazolo[5,4-b]pyridin-2-yl)nicotinamide

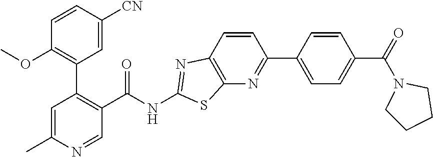 [SMILES: COc1ccc(C#N)cc1-c1cc(C)ncc1C(=O)Nc1nc2ccc(-c3ccc(C(=O)N4CCCC4)cc3)nc2s1]

The title compound (23.9 mg, 22%) was prepared in a manner similar to Example 73 step 5 from 4-(2-(4-(5-cyano-2-methoxyphenyl)-6-methylnicotinamido)thiazolo[5,4-b]pyridin-5-yl)benzoic acid and pyrrolidine. ¹H NMR (400 MHz, d6-DMSO) δ: 13.02 (s, 1H), 8.81 (s, 1H), 8.24-8.08 (s, 4H), 7.93-7.87 (m, 2H), 7.70-7.62 (m, 2H), 7.46 (s, 1H), 7.24-7.15 (m, 1H), 3.61 (s, 3H), 3.53-3.34 (m, 4H), 2.49 (s, 3H), 1.90-1.80 (s, 4H). LC-MS (M+H)⁺=575.1.

### Example 86: 4-(5-cyano-2-methoxyphenyl)-N-(5-(4-(dimethylcarbamoyl)phenyl)thiazolo[5,4-b]pyridin-2-yl)-6-methylnicotinamide

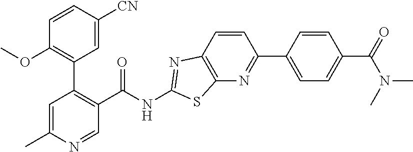

The title compound (18.7 mg, 18%) was prepared in a manner similar to that described in Example 73 step 5 from 4-(2-(4-(5-cyano-2-methoxyphenyl)-6-methylnicotinamido)thiazolo[5,4-b]pyridin-5-yl)benzoic acid and dimethylamine. 1H NMR (400 MHz, d6-DMSO) δ: 13.01 (s, 1H), 8.81 (s, 1H), 8.24-8.08 (s, 4H), 7.93-7.87 (m, 2H), 7.70-7.62 (m, 2H), 7.46 (s, 1H), 7.24-7.15 (m, 1H), 3.61 (s, 3H), 3.05-2.90 (m, 6H), 2.61 (s, 3H). LC-MS (M+H)⁺=549.1.

### Example 87: N-(5-(4-cyanophenyl)thiazolo[5,4-b]pyridin-2-yl)-4-(2-methoxy-6-methylphenyl)-6-methylnicotinamide

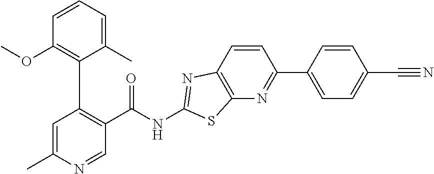 [SMILES: COc1cccc(C)c1-c1cc(C)ncc1C(=O)Nc1nc2ccc(-c3ccc(C#N)cc3)nc2s1]

### Step 1: 2-bromo-1-methoxy-3-methylbenzene

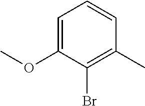

To the solution of 2-bromo-3-methylphenol (2.0 g, 10.6 mmol) in acetonitrile (25 mL) were added KOH (898 mg, 16.0 mmol), CH₃I (7.6 g, 53.5 mmol). The reaction mixture was stirred at 25° C. for 14 h. The mixture was diluted with ethyl acetate (50 mL), washed with brine (30 mL), dried over sodium sulfate, filtered and concentrated under reduce pressure. The residue was purified by silica gel column chromatography, eluting with ethyl acetate in petroleum ether (50%, v/v) to give title compound (1.8 g, 83%) which was used for the next step without further purification.

### Step 2: 2-(2-methoxy-6-methylphenyl)-4,4,5,5-tetramethyl-1,3,2-dioxaborolane

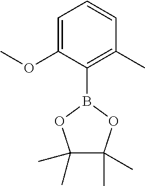

To a solution of 2-bromo-1-methoxy-3-methylbenzene (1.0 g, 5.0 mmol) in THF (25 mL) was added n-BuLi (2.8 mL, 7.5 mmol, 2.5 M). The reaction mixture was stirred at −75° C. for 0.5 h under nitrogen before addition of 2-isopropoxy-4,4,5,5-tetramethyl-1,3,2-dioxaborolane (2.3 g, 12.5 mmol). The reaction mixture was stirred at room temperature for 14 h before diluted with ethyl acetate (50 mL). The solution was washed with brine (30 mL), dried over sodium sulfate, filtered and concentrated under reduce pressure. The residue was purified by silica gel column chromatography, eluting with ethyl acetate in petroleum ether (50%, v/v) to give the title compound (1.0 g, 82%).

### Step 3: methyl 4-(2-methoxy-6-methylphenyl)-6-methylnicotinate

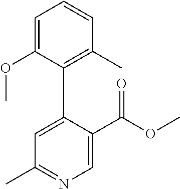 [SMILES: COC(=O)c1cnc(C)cc1-c1c(C)cccc1OC]

To a solution of 2-(2-methoxy-6-methylphenyl)-4,4,5,5-tetramethyl-1,3,2-dioxaborolane (400 mg, 1.6 mmol) and methyl 4-chloro-6-methylnicotinate (249 mg, 1.3 mmol) in dioxane (8 mL) and water (2 mL) was added 1,1′-bis(diphenylphosphino)ferrocene]dichloropalladium(II) (98 mg, 0.13 mmol) and K₂CO₃ (370 mg, 2.7 mmol). The mixture was stirred at 90° C. for 14 h under nitrogen. The reaction mixture was cooled to room temperature and diluted with ethyl acetate (30 mL), washed with brine (30 mL), dried with over sodium sulfate, and concentrated under reduced pressure. The residue was purified by silica gel column chromatography, eluting with ethyl acetate in petroleum ether (50%, v/v) to give the title compound (285 mg, 81%). LC-MS (M+H)⁺=272.1.

### Step 4: 4-(2-methoxy-6-methylphenyl)-6-methylnicotinic acid

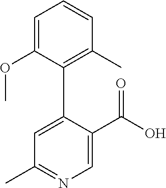 [SMILES: COc1cccc(C)c1-c1cc(C)ncc1C(=O)O]

To a solution of methyl 4-(2-methoxy-6-methylphenyl)-6-methylnicotinate (240 mg, 0.89 mmol) in THF (2 mL), methanol (2 mL) and water (2 mL) was added LiOH (84 mg, 3.5 mmol). The reaction mixture was stirred at room temperature for 4 h. The mixture was diluted with chloroform and isopropyl alcohol (40 mL), washed with brine (20 mL), dried over sodium sulfate, concentrated under reduce pressure to give compound 1-4 (206 mg, 90%). LC-MS (M+H)⁺=258.1.

### Step 5: N-(5-(4-cyanophenyl)thiazolo[5,4-b]pyridin-2-yl)-4-(2-methoxy-6-methylphenyl)-6-methylnicotinamide

The title compound (10.5 mg, 11%) was prepared in a manner similar to that in Example 11 step 3 from 4-(2-aminothiazolo[5,4-b]pyridin-5-yl)benzonitrile and 4-(2-methoxy-6-methylphenyl)-6-methylnicotinic acid. 1H NMR (400 MHz, d6-DMSO) δ: 13.00 (s, 1H), 8.85 (s, 1H), 8.35-8.28 (m, 2H), 8.26-8.21 (m, 2H), 8.02-7.89 (m, 2H), 7.15-7.22 (s, 2H), 6.97-6.85 (m, 2H), 3.53 (s, 3H), 2.49 (s, 3H), 2.09 (s, 3H). LC-MS (M+H)⁺=492.1.

### Example 88: 5-(2-methoxyphenyl)-N-(5-(4-(pyrrolidine-1-carbonyl)phenyl)thiazolo[5,4-b]pyridin-2-yl)pyridazine-4-carboxamide

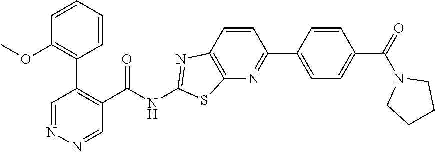 [SMILES: COc1ccccc1-c1cnncc1C(=O)Nc1nc2ccc(-c3ccc(C(=O)N4CCCC4)cc3)nc2s1]

### Step 1: methyl 4-(2-(5-(2-methoxyphenyl)pyridazine-4-carboxamido)thiazolo[5,4-b]pyridin-5-yl)benzoate

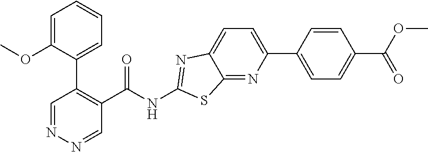

The title compound (1.54 g, 66%) was prepared in a manner similar to that in Example 25 step 3 from 5-(2-methoxyphenyl)pyridazine-4-carboxylic acid and methyl 4-(2-aminothiazolo[5,4-b]pyridin-5-yl)benzoate. LC-MS (M+H)⁺=498.1.

### Step 2: 4-(2-(5-(2-methoxyphenyl)pyridazine-4-carboxamido)thiazolo[5,4-b]pyridin-5-yl)benzoic acid

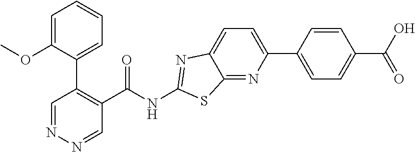 [SMILES: COc1ccccc1-c1cnncc1C(=O)Nc1nc2ccc(-c3ccc(C(=O)O)cc3)nc2s1]

The title compound (0.4 g, 95%) was prepared in a manner similar to that in Example 1 step 3 from methyl 4-(2-(5-(2-methoxyphenyl)pyridazine-4-carboxamido)thiazolo[5,4-b]pyridin-5-yl)benzoate. LC-MS (M+H)⁺=484.1.

### Step 3: 5-(2-methoxyphenyl)-N-(5-(4-(pyrrolidine-1-carbonyl)phenyl)thiazolo[5,4-b]pyridin-2-yl)pyridazine-4-carboxamide

The title compound (1.1 g, 97%) was prepared in a manner similar to that in Example 73 step 5 from 4-(2-(5-(2-methoxyphenyl)pyridazine-4-carboxamido)thiazolo[5,4-b]pyridin-5-yl)benzoic acid and pyrrolidine. 1H NMR (400 MHz, DMSO-d6) δ 13.30 (s, 1H), 9.50 (d, J=0.6 Hz, 1H), 9.41 (s, 1H), 8.28-8.07 (m, 4H), 7.65 (d, J=8.3 Hz, 2H), 7.57-7.44 (m, 2H), 7.15 (t, J=7.4 Hz, 1H), 7.07 (d, J=8.3 Hz, 1H), 3.57 (s, 3H), 3.49 (t, J=6.8 Hz, 2H), 3.44 (t, J=6.2 Hz, 2H), 1.95-1.78 (m, 4H). LC-MS (M+H)⁺=537.4.

### Example 89: 4-(5-cyano-2-methoxyphenyl)-6-methyl-N-(5-(3-methyl-4-(N-methylmethylsulfonamido)phenyl)thiazolo[5,4-b]pyridin-2-yl)nicotinamide

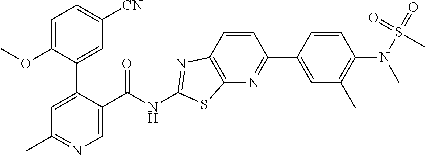

### Step 1: N-methyl-N-(2-methyl-4-(4,4,5,5-tetramethyl-1,3,2-dioxaborolan-2-yl)phenyl)methanesulfonamide

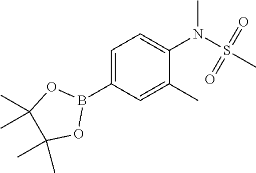

The title compound (560 mg, 99%) was prepared in a manner similar to that in Example 28 step 2 from N-(4-bromo-2-methylphenyl)-N-methylmethanesulfonamide and bis(pinacolato)diboron. LC-MS (M+H)⁺=326.2.

### Step 2: 4-(5-cyano-2-methoxyphenyl)-6-methyl-N-(5-(3-methyl-4-(N-methylmethylsulfonamido)phenyl)thiazolo[5,4-b]pyridin-2-yl)nicotinamide

The title compound (26 mg, 42%) was prepared in a manner similar to that in Example 78 step 3 from N-(5-bromothiazolo[5,4-b]pyridin-2-yl)-4-(5-cyano-2-methoxyphenyl)-6-methylnicotinamide and N-methyl-N-(2-methyl-4-(4,4,5,5-tetramethyl-1,3,2-dioxaborolan-2-yl)phenyl)methanesulfonamide. 1H NMR (400 MHz, d6-DMSO) δ: 12.99 (s, 1H), 8.80 (s, 1H), 8.19 (d, J=8.7 Hz, 1H), 8.12-8.05 (m, 2H), 8.00 (d, J=8.5 Hz, 1H), 7.95-7.87 (m, 2H), 7.56 (d, J=8.4 Hz, 1H), 7.44 (s, 1H), 7.18 (d, J=9.1 Hz, 1H), 3.60 (s, 3H), 3.19 (s, 3H), 3.10 (s, 3H), 2.60 (s, 3H), 2.41 (s, 3H). LC-MS (M+H)⁺=599.5.

### Example 90: 4-(5-cyano-2-methoxyphenyl)-N-(5-(3-fluoro-4-(methylsulfonamido)phenyl)thiazolo[5,4-b]pyridin-2-yl)-6-methylnicotinamide

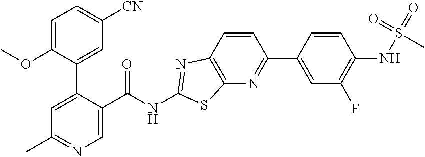

### Step 1: N-(2-fluoro-4-(4,4,5,5-tetramethyl-1,3,2-dioxaborolan-2-yl)phenyl)methanesulfonamide

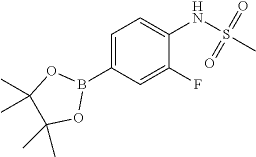

The title compound (550 mg, 99%) was prepared in a manner similar to that in Example 28 step 2 from N-(4-bromo-2-fluorophenyl)methanesulfonamide and bis(pinacolato)diboron. LC-MS (M+H)⁺=316.1.

### Step 2: 4-(5-cyano-2-methoxyphenyl)-N-(5-(3-fluoro-4-(methylsulfonamido)phenyl)thiazolo[5,4-b]pyridin-2-yl)-6-methylnicotinamide

The title compound (24 mg, 38%) was prepared in a manner similar to that in Example 78 step 3 from N-(5-bromothiazolo[5,4-b]pyridin-2-yl)-4-(5-cyano-2-methoxyphenyl)-6-methylnicotinamide and N-(2-fluoro-4-(4,4,5,5-tetramethyl-1,3,2-dioxaborolan-2-yl)phenyl)methanesulfonamide. 1H NMR (400 MHz, d6-DMSO) δ:13.01 (s, 1H), 9.79 (s, 1H), 8.80 (s, 1H), 8.20 (d, J=8.5 Hz, 1H), 8.12 (d, J=8.6 Hz, 1H), 8.06-7.96 (m, 2H), 7.94-7.87 (m, 2H), 7.53 (t, J=8.4 Hz, 1H), 7.44 (s, 1H), 7.18 (d, J=9.3 Hz, 1H), 3.60 (s, 3H), 3.10 (s, 3H), 2.60 (s, 3H). LC-MS (M+H)⁺=589.2.

### Example 91: N-(5-(4-cyanophenyl)thiazolo[5,4-b]pyridin-2-yl)-4-(2,6-dimethoxyphenyl)-6-methylnicotinamide

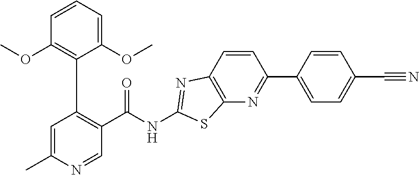 [SMILES: COc1cccc(OC)c1-c1cc(C)ncc1C(=O)Nc1nc2ccc(-c3ccc(C#N)cc3)nc2s1]

### Step 1: methyl 4-(2,6-dimethoxyphenyl)-6-methylnicotinate

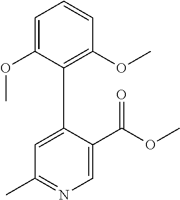 [SMILES: COC(=O)c1cnc(C)cc1-c1c(OC)cccc1OC]

The title compound (289 mg, 80%) was prepared in a manner similar to that described in Example 2 step 2 from methyl 4-chloro-6-methylnicotinate and 2-(2,6-dimethoxyphenyl)-4,4,5,5-tetramethyl-1,3,2-dioxaborolane. LC-MS (M+H)⁺=288.1

### Step 2: 4-(2,6-dimethoxyphenyl)-6-methylnicotinic acid

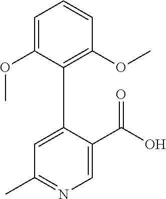 [SMILES: COc1cccc(OC)c1-c1cc(C)ncc1C(=O)O]

The title compound (253 mg, 89%) was prepared in a manner similar to that described in Example 1 step 3 from methyl 4-(2,6-dimethoxyphenyl)-6-methylnicotinate. LC-MS (M+H)⁺=274.1

### Step 3: N-(5-(4-cyanophenyl)thiazolo[5,4-b]pyridin-2-yl)-4-(2,6-dimethoxyphenyl)-6-methylnicotinamide

The title compound (9.0 mg, 3.8%) was prepared in a manner similar to that described in Example 11 step 3 from 4-(2,6-dimethoxyphenyl)-6-methylnicotinic acid and 4-(2-aminothiazolo[5,4-b]pyridin-5-yl)benzonitrile. 1H NMR (400 MHz, d6-DMSO) δ: 12.98 (s, 1H), 8.85 (s, 1H), 8.35-8.28 (m, 2H), 8.26-8.21 (m, 2H), 8.02-7.89 (m, 2H), 7.36-7.20 (s, 2H), 6.75-6.65 (m, 2H), 3.61 (s, 6H), 2.49 (s, 3H). LC-MS (M+H)⁺=508.1.

### Example 92: N-(5-(4-(dimethylcarbamoyl)phenyl)thiazolo[5,4-b]pyridin-2-yl)-5-(2-methoxyphenyl)pyridazine-4-carboxamide

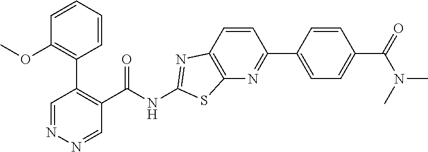

The title compound (50 mg, 47%) was prepared in a manner similar to that in Example 73 step 5 from 4-(2-(5-(2-methoxyphenyl)pyridazine-4-carboxamido)thiazolo[5,4-b]pyridin-5-yl)benzoic acid and dimethylamine. 1H NMR (400 MHz, DMSO-d6) δ 13.30 (s, 1H), 9.50 (d, J=0.7 Hz, 1H), 9.43 (s, 1H), 8.30-8.09 (m, 4H), 7.60-7.44 (m, 4H), 7.16 (t, J=7.5 Hz, 1H), 7.07 (d, J=8.3 Hz, 1H), 3.57 (s, 3H), 2.99 (d, J=17.9 Hz, 6H). LC-MS (M+H)⁺=511.5.

### Example 93: 5-(2-methoxyphenyl)-N-(5-(4-(2-oxopyrrolidin-1-yl)phenyl)thiazolo[5,4-b]pyridin-2-yl)pyridazine-4-carboxamide

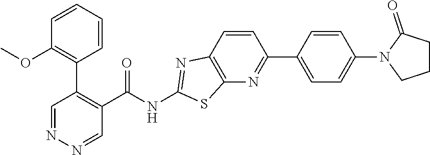 [SMILES: COc1ccccc1-c1cnncc1C(=O)Nc1nc2ccc(-c3ccc(N4CCCC4=O)cc3)nc2s1]

The title compound (43 mg, 36%) was prepared in a manner similar to that in Example 78 step 3 from N-(5-bromothiazolo[5,4-b]pyridin-2-yl)-5-(2-methoxyphenyl)pyridazine-4-carboxamide and 1-(4-(4,4,5,5-tetramethyl-1,3,2-dioxaborolan-2-yl)phenyl)pyrrolidin-2-one. 1H NMR (300 MHz, d6-DMSO) δ 9.9.49 (s, 1H), 9.43 (s, 1H), 8.25-8.02 (m, 4H), 7.86-7.77 (m, 2H), 7.59-7.44 (m, 2H), 7.21-7.02 (m, 2H), 3.90 (t, J=7.0 Hz, 2H), 3.55 (s, 3H), 2.59-2.51 (m, 2H), 2.17-2.04 (m, 2H). LC-MS (M+H)⁺=523.1.

### Example 94: N-ethyl-2-(5-(2-methoxyphenyl)pyridazine-4-carboxamido)thiazolo[5,4-b]pyridine-5-carboxamide

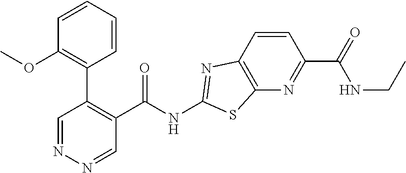 [SMILES: CCNC(=O)c1ccc2nc(NC(=O)c3cnncc3-c3ccccc3OC)sc2n1]

### Step 1: methyl 2-bromothiazolo[5,4-b]pyridine-5-carboxylate

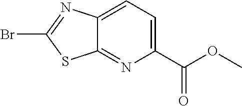

The title compound (252 mg, 74%) was prepared in a manner similar to that in Example 69 step 2 from methyl 2-aminothiazolo[5,4-b]pyridine-5-carboxylate. LC-MS (M+H)⁺=272.8.

### Step 2: methyl 2-(5-(2-methoxyphenyl)pyridazine-4-carboxamido)thiazolo[5,4-b]pyridine-5-carboxylate

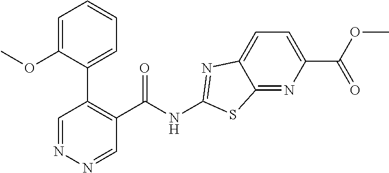

The title compound (274 mg, 73%) was prepared in a manner similar to that in Example 69 step 4 from methyl 2-bromothiazolo[5,4-b]pyridine-5-carboxylate and 5-(2-methoxyphenyl)pyridazine-4-carboxamide. LC-MS (M+H)⁺=421.9.

### Step 3: 2-(5-(2-methoxyphenyl)pyridazine-4-carboxamido)thiazolo[5,4-b]pyridine-5-carboxylic acid

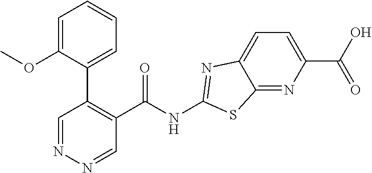 [SMILES: COc1ccccc1-c1cnncc1C(=O)Nc1nc2ccc(C(=O)O)nc2s1]

The title compound (163 mg, 61%) was prepared in a manner similar to that in Example 1 step 3 from methyl 2-(5-(2-methoxyphenyl)pyridazine-4-carboxamido)thiazolo[5,4-b]pyridine-5-carboxylate. LC-MS (M+H)⁺=408.0.

### Step 5: N-ethyl-2-(5-(2-methoxyphenyl)pyridazine-4-carboxamido)thiazolo[5,4-b]pyridine-5-carboxamide

The title compound (25 mg, 79%) was prepared in a manner similar to that in Example 73 step 5 from 2-(5-(2-methoxyphenyl)pyridazine-4-carboxamido)thiazolo[5,4-b]pyridine-5-carboxylic acid and ethylamine hydrochloride. 1H NMR (400 MHz, d6-DMSO) δ 13.46 (brs, 1H), 9.50 (s, 1H), 9.42 (s, 1H), 8.91 (t, J=6.0 Hz, 1H), 8.28-8.11 (m, 2H), 7.58-7.40 (m, 2H), 7.20-7.03 (m, 2H), 3.55 (s, 3H), 3.38-3.29 (m, 2H), 1.14 (t, J=7.2 Hz, 3H). LC-MS (M+H)⁺=435.0.

### Example 95: N-(5-ethoxythiazolo[5,4-b]pyridin-2-yl)-5-(2-methoxyphenyl)pyridazine-4-carboxamide

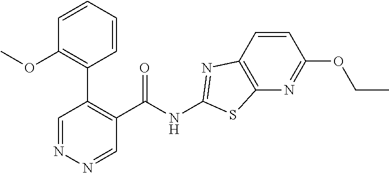 [SMILES: CCOc1ccc2nc(NC(=O)c3cnncc3-c3ccccc3OC)sc2n1]

### Step 1: 2-aminothiazolo[5,4-b]pyridin-5-ol

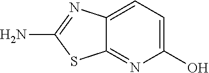 [SMILES: Nc1nc2ccc(O)nc2s1]

A mixture of 5-methoxythiazolo[5,4-b]pyridin-2-amine (285 mg, 1.573 mmol) in HBr in water (30.00 mL, 48%) was stirred for 16 h at 100° C. under nitrogen atmosphere. After consumption of starting material, the mixture was basified to pH 8 with saturated aqueous NaHCO₃. The precipitated solids were collected by filtration and washed with water (2×5 mL) to yield the title compound (198 mg, 75%). LC-MS (M+H)⁺=168.2.

### Step 2: 5-ethoxythiazolo[5,4-b]pyridin-2-amine

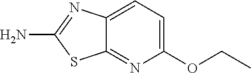

To a mixture of 2-aminothiazolo[5,4-b]pyridin-5-ol (148 mg, 0.886 mmol) and ethyl iodide (165 mg, 1.063 mmol) in N,N-dimethylformamide (3 mL) was added CsF (538 mg, 3.544 mmol) at room temperature under nitrogen atmosphere. The resulting mixture was stirred for 16 h at room temperature under nitrogen atmosphere. After consumption of starting material, the reaction was then quenched by the addition of water (10 mL) at room temperature. The resulting solution was extracted with dichloromethane (3×10 mL). The organic phases were combined, washed with brine and dried over Na₂SO₄. The solvent was concentrated under reduced pressure and the residue was purified by flash chromatography eluting with ethyl acetate in petroleum ether (0% to 40% gradient) to yield the title compound (37 mg, 21%). LC-MS (M+H)⁺=196.2.

### Step 3: N-(5-ethoxythiazolo[5,4-b]pyridin-2-yl)-5-(2-methoxyphenyl)pyridazine-4-carboxamide

The title compound (21 mg, 36%) was prepared in a manner similar to that in Example 81 step 2 from 5-ethoxythiazolo[5,4-b]pyridin-2-amine and 5-(2-methoxyphenyl)pyridazine-4-carboxylic acid. 1H NMR (300 MHz, d6-DMSO) δ 13.06 (brs, 1H), 9.47 (d, J=1.2 Hz, 1H), 9.40 (s, 1H), 8.06 (m, 12.0 Hz, 1H), 7.55-7.41 (m, 2H), 7.18-7.00 (m, 2H), 6.94-6.85 (m, 1H), 4.33 (q, J=7.0 Hz, 2H), 3.53 (s, 3H), 1.33 (t, J=7.0 Hz, 3H). LC-MS (M+H)⁺=408.0.

### Example 96: N-(5-(4-chloro-2-methoxyphenyl)thiazolo[5,4-b]pyridin-2-yl)-5-(2-methoxyphenyl)pyridazine-4-carboxamide

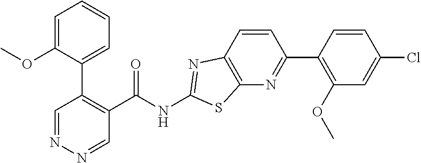 [SMILES: COc1cc(Cl)ccc1-c1ccc2nc(NC(=O)c3cnncc3-c3ccccc3OC)sc2n1]

### Step 1: N-(5-bromothiazolo[5,4-b]pyridin-2-yl)-5-(2-methoxyphenyl)pyridazine-4-carboxamide

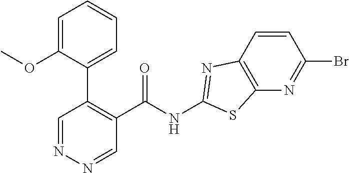 [SMILES: COc1ccccc1-c1cnncc1C(=O)Nc1nc2ccc(Br)nc2s1]

The reaction mixture of 5-(2-methoxyphenyl)pyridazine-4-carboxylic acid (1 g, 4.35 mmol), 5-bromothiazolo[5,4-b]pyridin-2-amine (1 g, 4.35 mmol), T₃P (10 mL) in pyridine (10 mL) under nitrogen was stirred at 80° C. for 14 h. The solvent was evaporated under vacuum. Water (30 mL) was added. The residue was extracted with ethyl acetate (20 mL×3). The combined organic layer was washed with brine, dried over Na₂SO₄, filtered, and concentrated in vacuo afford crude product (1.6 g, 83%). LC-MS (M+H)⁺=441.9, 443.9.

### Step 2: N-(5-(4-chloro-2-methoxyphenyl)thiazolo[5,4-b]pyridin-2-yl)-5-(2-methoxyphenyl)pyridazine-4-carboxamide

The reaction mixture of N-(5-bromothiazolo[5,4-b]pyridin-2-yl)-5-(2-methoxyphenyl)pyridazine-4-carboxamide (50 mg, 0.113 mmol), (4-chloro-2-methoxyphenyl)boronic acid (21 mg, 0.17 mmol), 1,1′-bis(diphenylphosphino)ferrocene]dichloropalladium(II) (10 mg, 0.01 mmol) and K₂CO₃ (31 mg, 0.226 mmol) in 1,4-dioxane/water (10 mL/1 mL) under nitrogen was stirred at 90° C. for 14 h. The mixture was filtered and the filtrate was evaporated. The residue was purified using prep-HPLC to give the title compound (18 mg, 34%). ¹H NMR (400 MHz, d6-DMSO) δ 13.26 (s, 1H), 9.50 (s, 1H), 9.43 (s, 1H), 8.17 (d, J=8.5 Hz, 1H), 7.97 (d, J=8.6 Hz, 1H), 7.81 (d, J=8.3 Hz, 1H), 7.57-7.51 (m, 1H), 7.48 (d, J=7.4 Hz, 1H), 7.27 (d, J=1.9 Hz, 1H), 7.19-7.12 (m, 2H), 7.07 (d, J=8.2 Hz, 1H), 3.89 (s, 3H), 3.56 (s, 3H). LC-MS (M+H)⁺=504.1.

### Example 97: N-(5-(4-chloro-2-methylphenyl)thiazolo[5,4-b]pyridin-2-yl)-5-(2-methoxyphenyl)pyridazine-4-carboxamide

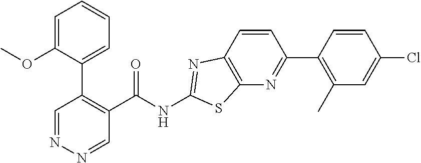 [SMILES: COc1ccccc1-c1cnncc1C(=O)Nc1nc2ccc(-c3ccc(Cl)cc3C)nc2s1]

The title compound (32 mg, 14%) was prepared in a manner similar to Step 1-2 from N-(5-bromothiazolo[5,4-b]pyridin-2-yl)-5-(2-methoxyphenyl)pyridazine-4-carboxamide and (4-chloro-2-methylphenyl)boronic acid. 1H NMR (400 MHz, d6-DMSO) δ 13.28 (s, 1H), 9.53-9.47 (m, 1H), 9.43 (s, 1H), 8.23 (d, J=8.4 Hz, 1H), 7.67 (d, J=8.4 Hz, 1H), 7.54 (dd, J=7.6, 1.5 Hz, 1H), 7.52-7.46 (m, 2H), 7.44 (d, J=1.9 Hz, 1H), 7.38 (dd, J=8.3, 2.2 Hz, 1H), 7.15 (t, J=7.4 Hz, 1H), 7.07 (d, J=8.3 Hz, 1H), 3.57 (s, 3H), 2.37 (s, 3H). LC-MS (M+H)⁺=488.1.

### Example 98: N-(5-(4-chlorophenoxy)thiazolo[5,4-b]pyridin-2-yl)-5-(2-methoxyphenyl)pyridazine-4-carboxamide

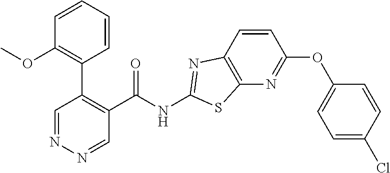 [SMILES: COc1ccccc1-c1cnncc1C(=O)Nc1nc2ccc(Oc3ccc(Cl)cc3)nc2s1]

### Step 1: 2-chloro-6-(4-chlorophenoxy)-3-nitropyridine & 6-chloro-2-(4-chlorophenoxy)-3-nitropyridine

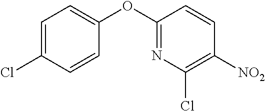 [SMILES: O=[N+]([O-])c1ccc(Oc2ccc(Cl)cc2)nc1Cl]

To a mixture of 2,6-dichloro-3-nitropyridine (1 g, 5.182 mmol) and 4-chlorophenol (0.67 g, 5.182 mmol) in DMSO (30 mL) was added Cs₂CO₃ (1.69 g, 5.182 mmol) at room temperature under nitrogen atmosphere. The resulting mixture was stirred for 16 h at room temperature under nitrogen atmosphere. After consumption of starting material, the reaction was then quenched by the addition of water (35 mL) at room temperature. The resulting solution was extracted with ethyl acetate (3×30 mL). The organic phases were combined, washed with brine and dried over Na₂SO₄. The solvent was concentrated under reduced pressure and the residue was purified by flash chromatography eluting with dichloromethane in petroleum ether (0% to 60% gradient) to yield a mixture of 2-chloro-6-(4-chlorophenoxy)-3-nitropyridine & 6-chloro-2-(4-chlorophenoxy)-3-nitropyridine (748 mg, 50%). LC-MS (M+H)⁺=284.9.

### Step 2: 2-chloro-6-(4-chlorophenoxy)pyridin-3-amine

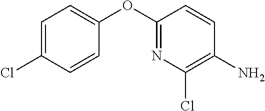 [SMILES: Nc1ccc(Oc2ccc(Cl)cc2)nc1Cl]

To the above mixture of 2-chloro-6-(4-chlorophenoxy)-3-nitropyridine and 6-chloro-2-(4-chlorophenoxy)-3-nitropyridine (740 mg, 2.596 mmol) in methanol (24 mL)/water (8 mL) were added Fe (49 mg, 0.875 mmol) and NH₄Cl (694 mg, 12.980 mmol) at room temperature. The resulting mixture was stirred for 3 h at 80° C. After consumption of starting material, the resulting mixture was concentrated under reduced pressure and the residue was purified by flash chromatography eluting with dichloromethane in petroleum ether (0% to 50% gradient) to yield the title compound (247 mg, 37%). 1H NMR (300 MHz, d6-DMSO) δ 7.35-7.24 (m, 2H), 7.17 (d, J=8.4 Hz, 1H), 6.98-6.87 (m, 2H), 6.76 (d, J=8.4 Hz, 1H), 5.25 (s, 2H).

### Step 3: N-((2-chloro-6-(4-chlorophenoxy)pyridin-3-yl)carbamothioyl)benzamide

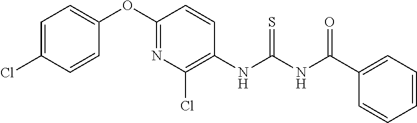 [SMILES: O=C(NC(=S)Nc1ccc(Oc2ccc(Cl)cc2)nc1Cl)c1ccccc1]

A mixture of 2-chloro-6-(4-chlorophenoxy)pyridin-3-amine (178 mg, 0.713 mmol) and benzoyl isothiocyanate (114 mg, 0.713 mmol) in acetone (5.0 mL) was stirred for 16 h at room temperature under nitrogen atmosphere. After consumption of starting material, the resulting mixture was concentrated under reduced pressure. The residue was purified by trituration with petroleum ether (3×5 mL) to yield the title compound (208 mg, 71%). LC-MS (M+H)⁺=417.9.

### Step 4: 5-(4-chlorophenoxy)thiazolo[5,4-b]pyridin-2-amine

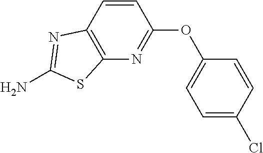 [SMILES: Nc1nc2ccc(Oc3ccc(Cl)cc3)nc2s1]

To a solution of N-((2-chloro-6-(4-chlorophenoxy)pyridin-3-yl)carbamothioyl)benzamide (196 mg, 0.469 mmol) in methanol (2.0 mL) treated with NaOH (10M) (250 uL, 0.070 mmol) dropwise at 0° C. The resulting mixture was stirred for 16 h at 75° C. under nitrogen atmosphere. After consumption of starting material, the resulting mixture was concentrated under reduced pressure and the residue was purified by flash chromatography eluting with ethyl acetate in dichloromethane (0% to 25% gradient) to yield the title compound (78 mg, 60%). LC-MS (M+H)⁺=277.9.

### Step 5: N-(5-(4-chlorophenoxy)thiazolo[5,4-b]pyridin-2-yl)-5-(2-methoxyphenyl)pyridazine-4-carboxamide

The title compound (46 mg, 37%) was prepared in a manner similar to that in Example 81 step 2 from 5-(4-chlorophenoxy)thiazolo[5,4-b]pyridin-2-amine and 5-(2-methoxyphenyl)pyridazine-4-carboxylic acid. 1H NMR (300 MHz, d6-DMSO) δ 13.16 (brs, 1H), 9.46 (s, 1H), 9.41 (s, 1H), 8.28-8.19 (m, 1H), 7.56-7.41 (m, 4H), 7.29-7.10 (m, 4H), 7.09-7.00 (m, 1H), 3.53 (s, 3H). LC-MS (M+H)⁺=490.0.

### Example 99: N-(5-(4-chloro-2-(trifluoromethyl)phenyl)thiazolo[5,4-b]pyridin-2-yl)-5-(2-methoxyphenyl)pyridazine-4-carboxamide

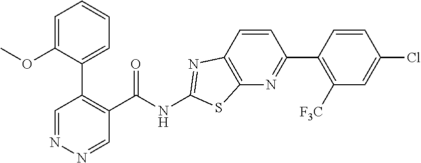 [SMILES: COc1ccccc1-c1cnncc1C(=O)Nc1nc2ccc(-c3ccc(Cl)cc3C(F)(F)F)nc2s1]

The title compound (117 mg, 52%) was prepared in a manner similar to Example 96 step 2 from N-(5-bromothiazolo[5,4-b]pyridin-2-yl)-5-(2-methoxyphenyl)pyridazine-4-carboxamide and (4-chloro-2-(trifluoromethyl)phenyl)boronic acid. 1H NMR (400 MHz, d6-DMSO) δ 13.35 (s, 1H), 9.50 (s, 1H), 9.43 (s, 1H), 8.26 (d, J=8.4 Hz, 1H), 7.97 (d, J=2.0 Hz, 1H), 7.88 (dd, J=8.3, 2.0 Hz, 1H), 7.68 (d, J=8.3 Hz, 1H), 7.64 (d, J=8.4 Hz, 1H), 7.53 (d, J=7.6 Hz, 1H), 7.52-7.45 (m, 1H), 7.15 (t, J=7.5 Hz, 1H), 7.08 (d, J=8.3 Hz, 1H), 3.57 (s, 3H). LC-MS (M+H)⁺=542.1.

### Example 100: N-(5-(4-chloro-2-fluorophenyl)thiazolo[5,4-b]pyridin-2-yl)-5-(2-methoxyphenyl)pyridazine-4-carboxamide

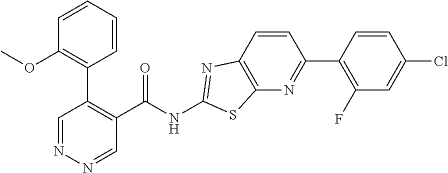 [SMILES: COc1ccccc1-c1cnncc1C(=O)Nc1nc2ccc(-c3ccc(Cl)cc3F)nc2s1]

The title compound (135 mg, 30%) was prepared in a manner similar to Example 96 step 2 from N-(5-bromothiazolo[5,4-b]pyridin-2-yl)-5-(2-methoxyphenyl)pyridazine-4-carboxamide and (4-chloro-2-fluorophenyl)boronic acid. 1H NMR (400 MHz, d6-DMSO) δ 13.35 (s, 1H), 9.51 (s, 1H), 9.43 (s, 1H), 8.26 (d, J=8.5 Hz, 1H), 8.04 (t, J=8.6 Hz, 1H), 7.92 (dd, J=8.5, 1.8 Hz, 1H), 7.62 (dd, J=11.2, 2.0 Hz, 1H), 7.55 (dd, J=7.6, 1.5 Hz, 1H), 7.49-7.44 (m, 2H), 7.16 (t, J=7.5 Hz, 1H), 7.07 (d, J=8.2 Hz, 1H), 3.56 (s, 3H). LC-MS (M+H)⁺=492.1.

### Example 101: N-(5-(4-chloro-2,6-dimethylphenyl)thiazolo[5,4-b]pyridin-2-yl)-5-(2-methoxyphenyl)pyridazine-4-carboxamide

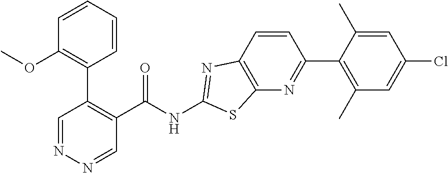 [SMILES: COc1ccccc1-c1cnncc1C(=O)Nc1nc2ccc(-c3c(C)cc(Cl)cc3C)nc2s1]

The title compound (22 mg, 10%) was prepared in a manner similar to Example 96 step 2 from N-(5-bromothiazolo[5,4-b]pyridin-2-yl)-5-(2-methoxyphenyl)pyridazine-4-carboxamide and (4-chloro-2,6-dimethylphenyl)boronic acid. ¹H NMR (400 MHz, d6-DMSO) δ 13.28 (s, 1H), 9.49 (s, 1H), 9.41 (s, 1H), 8.22 (d, J=8.1 Hz, 1H), 7.50 (dd, J=17.7, 7.8 Hz, 2H), 7.43 (d, J=8.4 Hz, 1H), 7.25 (s, 2H), 7.14 (t, J=7.5 Hz, 1H), 7.08 (d, J=8.3 Hz, 1H), 3.59 (s, 3H), 1.99 (s, 6H). LC-MS (M+H)⁺=502.1.

### Example 102: N-(5-(4-(isopropylcarbamoyl)phenyl)thiazolo[5,4-b]pyridin-2-yl)-5-(2-methoxyphenyl)pyridazine-4-carboxamide

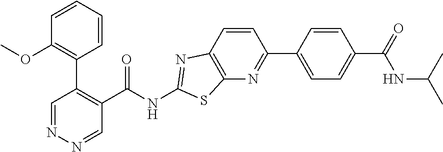 [SMILES: COc1ccccc1-c1cnncc1C(=O)Nc1nc2ccc(-c3ccc(C(=O)NC(C)C)cc3)nc2s1]

The title compound (50 mg, 45%) was prepared in a manner similar to that in Example 73 step 5 from 4-(2-(5-(2-methoxyphenyl)pyridazine-4-carboxamido)thiazolo[5,4-b]pyridin-5-yl)benzoic acid and propan-2-amine. 1H NMR (400 MHz, d6-DMSO) δ 13.31 (s, 1H), 9.50 (s, 1H), 9.43 (s, 1H), 8.31 (d, J=7.7 Hz, 1H), 8.28-8.16 (m, 4H), 7.98 (d, J=8.4 Hz, 2H), 7.55 (d, J=7.6 Hz, 1H), 7.52-7.47 (m, 1H), 7.19-7.13 (m, 1H), 7.07 (d, J=8.3 Hz, 1H), 4.18-4.08 (m, 1H), 3.56 (s, 3H), 1.19 (d, J=6.6 Hz, 6H). LC-MS (M+H)⁺=525.3.

### Example 103: 5-(2-methoxyphenyl)-N-(5-(4-(morpholine-4-carbonyl)phenyl)thiazolo[5,4-b]pyridin-2-yl)pyridazine-4-carboxamide

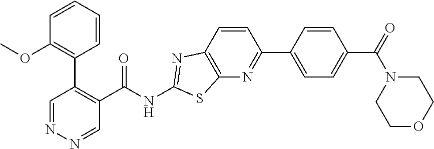 [SMILES: COc1ccccc1-c1cnncc1C(=O)Nc1nc2ccc(-c3ccc(C(=O)N4CCOCC4)cc3)nc2s1]

The title compound (50 mg, 43%) was prepared in a manner similar to that in Example 73 step 5 from 4-(2-(5-(2-methoxyphenyl)pyridazine-4-carboxamido)thiazolo[5,4-b]pyridin-5-yl)benzoic acid and morpholine. 1H NMR (400 MHz, dmso) δ 13.30 (s, 1H), 9.50 (s, 1H), 9.43 (s, 1H), 8.28-8.19 (m, 3H), 8.16 (d, J=8.6 Hz, 1H), 7.57-7.52 (m, 3H), 7.52-7.46 (m, 1H), 7.18-7.13 (m, 1H), 7.07 (d, J=8.2 Hz, 1H), 3.75-3.35 (m, 11H). LC-MS (M+H)⁺=553.3.

### Example 104: N-(5-(4-cyanophenyl)thiazolo[5,4-b]pyridin-2-yl)-4-(2-fluoro-6-methoxyphenyl)-6-methylnicotinamide

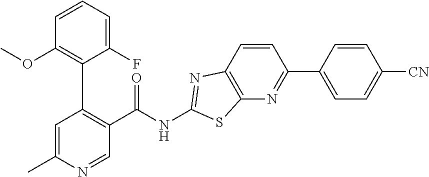 [SMILES: COc1cccc(F)c1-c1cc(C)ncc1C(=O)Nc1nc2ccc(-c3ccc(C#N)cc3)nc2s1]

### Step 1: 2-(2-fluoro-6-methoxyphenyl)-4,4,5,5-tetramethyl-1,3,2-dioxaborolane

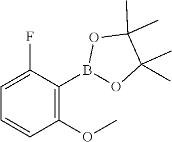 [SMILES: COc1cccc(F)c1B1OC(C)(C)C(C)(C)O1]

The title compound (876 mg, 71%) was prepared in a manner similar to that described in Example 87 step 2 from 2-bromo-1-fluoro-3-methoxybenzene.

### Step 2: methyl 4-(2-fluoro-6-methoxyphenyl)-6-methylnicotinate

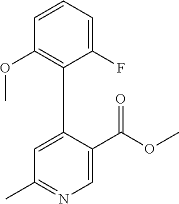

The title compound (341 mg, 78%) was prepared in a manner similar to that described in Example 2 step 2 from methyl 4-chloro-6-methylnicotinate and 2-(2-fluoro-6-methoxyphenyl)-4,4,5,5-tetramethyl-1,3,2-dioxaborolane. LC-MS (M+H)⁺=276.1.

### Step 3: 4-(2-fluoro-6-methoxyphenyl)-6-methylnicotinic acid

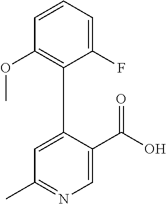 [SMILES: COc1cccc(F)c1-c1cc(C)ncc1C(=O)O]

The title compound (250 mg, 88%) was prepared in a manner similar to that described in Example 1 step 3 from methyl 4-(2-fluoro-6-methoxyphenyl)-6-methylnicotinate. LC-MS (M+H)⁺=262.1.

### Step 4: N-(5-(4-cyanophenyl)thiazolo[5,4-b]pyridin-2-yl)-4-(2-fluoro-6-methoxyphenyl)-6-methylnicotinamide

The title compound (24.5 mg, 11%) was prepared in a manner similar to that described in Example 11 step 3 from 4-(2-fluoro-6-methoxyphenyl)-6-methylnicotinic acid and 4-(2-aminothiazolo[5,4-b]pyridin-5-yl)benzonitrile. ¹H NMR (400 MHz, d6-DMSO) δ: 13.14 (s, 1H), 8.88 (s, 1H), 8.35-8.28 (m, 2H), 8.26-8.21 (m, 2H), 8.02-7.89 (m, 2H), 7.50-7.35 (m, 2H), 6.97-6.85 (m, 2H), 3.53 (s, 3H), 2.49 (s, 3H). LC-MS (M+H)⁺=496.1.

### Example 105: 4-(5-cyano-2-ethoxyphenyl)-N-(5-(4-cyanophenyl)thiazolo[5,4-b]pyridin-2-yl)-6-methylnicotinamide

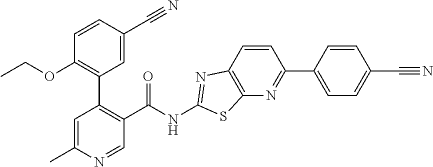 [SMILES: CCOc1ccc(C#N)cc1-c1cc(C)ncc1C(=O)Nc1nc2ccc(-c3ccc(C#N)cc3)nc2s1]

### Step 1: 3-bromo-4-ethoxybenzonitrile

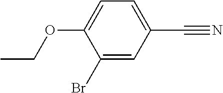

The mixture of 3-bromo-4-hydroxybenzonitrile (1.98 g, 10 mmol), EtI (1.87 g, 12 mmol) and K₂CO₃ (2.07 g, 15 mmol) in N,N-dimethylformamide (20 mL) were stirred at 80° C. for 3 hours. The mixture was diluted with ethyl acetate and washed by brine, the organic phase was dried and evaporated. The residue was purified by column chromatography, eluting with ethyl acetate in petroleum ether (17%, v/v) to give the title compound (2.3 g, 96%). LC-MS (M+H)⁺=226.3.

### Step 2: 4-ethoxy-3-(4,4,5,5-tetramethyl-1,3,2-dioxaborolan-2-yl)benzonitrile

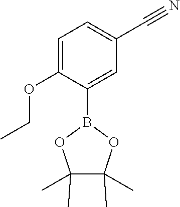 [SMILES: CCOc1ccc(C#N)cc1B1OC(C)(C)C(C)(C)O1]

The title compound (0.9 g, 33%) was prepared in a manner similar to that in Example 28 step 2 from 3-bromo-4-ethoxybenzonitrile and bis(pinacolato)diboron.

### Step 3: methyl 4-(5-cyano-2-ethoxyphenyl)-6-methylnicotinate

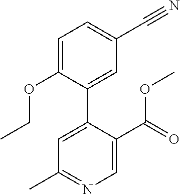 [SMILES: CCOc1ccc(C#N)cc1-c1cc(C)ncc1C(=O)OC]

The title compound (0.17 g, 60%) was prepared in a manner similar to that in Example 2 step 2 from 4-ethoxy-3-(4,4,5,5-tetramethyl-1,3,2-dioxaborolan-2-yl)benzonitrile and methyl 4-chloro-6-methylnicotinate. LC-MS (M+H)⁺=297.4.

### Step 4: 4-(5-cyano-2-ethoxyphenyl)-6-methylnicotinic acid

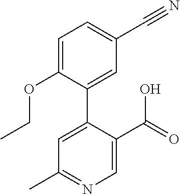 [SMILES: CCOc1ccc(C#N)cc1-c1cc(C)ncc1C(=O)O]

The title compound (150 mg, 93%) was prepared in a manner similar to Example 1 of Step 3 from methyl 4-(5-cyano-2-ethoxyphenyl)-6-methylnicotinate. LC-MS (M+H)⁺=283.4.

### Step 5: 4-(5-cyano-2-ethoxyphenyl)-N-(5-(4-cyanophenyl)thiazolo[5,4-b]pyridin-2-yl)-6-methylnicotinamide

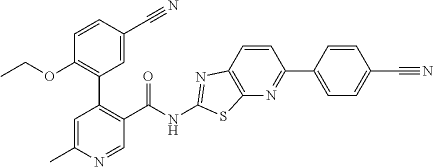 [SMILES: CCOc1ccc(C#N)cc1-c1cc(C)ncc1C(=O)Nc1nc2ccc(-c3ccc(C#N)cc3)nc2s1]

The title compound (10 mg, 3.7%) was prepared in a manner similar to Example 11 of Step 3 from 4-(5-cyano-2-ethoxyphenyl)-6-methylnicotinic acid and 4-(2-aminothiazolo[5,4-b]pyridin-5-yl)benzonitrile. 1H NMR (400 MHz, d6-DMSO) δ 13.08 (s, 1H), 8.84 (s, 1H), 8.34 (d, J=8.4 Hz, 2H), 8.24 (q, J=8.6 Hz, 2H), 7.98 (d, J=8.3 Hz, 2H), 7.89 (d, J=7.5 Hz, 2H), 7.42 (s, 1H), 7.16 (d, J=9.2 Hz, 1H), 4.00-3.80 (m, 2H), 2.61 (s, 3H), 1.09 (t, J=7.0 Hz, 3H). LC-MS (M+H)⁺=517.4.

### Example 106: 4-(5-cyano-2-methylphenyl)-N-(5-(4-cyanophenyl)thiazolo[5,4-b]pyridin-2-yl)-6-methylnicotinamide

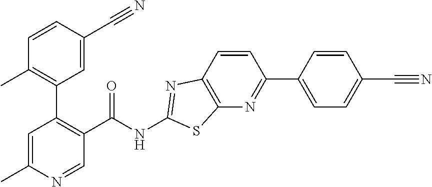 [SMILES: Cc1cc(-c2cc(C#N)ccc2C)c(C(=O)Nc2nc3ccc(-c4ccc(C#N)cc4)nc3s2)cn1]

### Step 1: methyl 4-(5-cyano-2-methylphenyl)-6-methylnicotinate

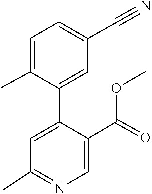 [SMILES: COC(=O)c1cnc(C)cc1-c1cc(C#N)ccc1C]

The title compound (290 mg, 40%) was prepared in a manner similar to Example 2 of Step 2 from (5-cyano-2-methylphenyl)boronic acid and methyl 4-chloro-6-methylnicotinate. LC-MS (M+H)⁺=267.4.

### Step 2: 4-(5-cyano-2-methylphenyl)-6-methylnicotinic acid

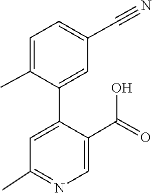 [SMILES: Cc1cc(-c2cc(C#N)ccc2C)c(C(=O)O)cn1]

The title compound (150 mg, 93%) was prepared in a manner similar to Example 1 of Step 3 from methyl 4-(5-cyano-2-methylphenyl)-6-methylnicotinate (290 mg, 1.09 mmol). LC-MS (M+H)⁺=253.6.

### Step 3: 4-(5-cyano-2-methylphenyl)-N-(5-(4-cyanophenyl)thiazolo[5,4-b]pyridin-2-yl)-6-methylnicotinamide

The title compound (50 mg, 21.4%) was prepared in a manner similar to Example 11 of Step 3 from 4-(5-cyano-2-methylphenyl)-6-methylnicotinic acid and 4-(2-aminothiazolo[5,4-b]pyridin-5-yl)benzonitrile. 1H NMR (400 MHz, d6-DMSO) δ 13.17 (s, 1H), 8.96 (s, 1H), 8.32 (d, J=8.4 Hz, 2H), 8.22 (q, J=8.6 Hz, 2H), 7.96 (d, J=8.4 Hz, 2H), 7.78 (dd, J=7.9, 1.5 Hz, 1H), 7.65 (d, J=1.3 Hz, 1H), 7.50 (d, J=8.0 Hz, 1H), 7.35 (s, 1H), 2.62 (s, 3H), 2.16 (s, 3H). LC-MS (M+H)⁺=487.4.

### Example 107: N-(5-(4-chlorophenethyl)thiazolo[5,4-b]pyridin-2-yl)-4-(5-cyano-2-methoxyphenyl)-6-methylnicotinamide

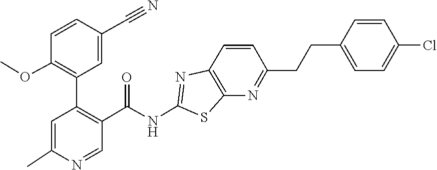 [SMILES: COc1ccc(C#N)cc1-c1cc(C)ncc1C(=O)Nc1nc2ccc(CCc3ccc(Cl)cc3)nc2s1]

### Step 1: N-(5-bromothiazolo[5,4-b]pyridin-2-yl)-4-(5-cyano-2-methoxyphenyl)-6-methylnicotinamide

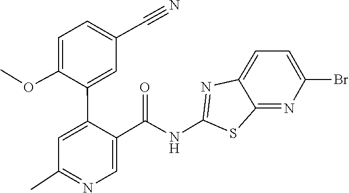 [SMILES: COc1ccc(C#N)cc1-c1cc(C)ncc1C(=O)Nc1nc2ccc(Br)nc2s1]

The title compound (0.5 g, 89%) was prepared in a manner similar to Example 11 of Step 3 from 4-(5-cyano-2-methoxyphenyl)-6-methylnicotinic acid and 5-bromothiazolo[5,4-b]pyridin-2-amine. LC-MS (M+H)⁺=480.4.

### Step 2: N-(5-((4-chlorophenyl)ethynyl)thiazolo[5,4-b]pyridin-2-yl)-4-(5-cyano-2-methoxyphenyl)-6-methylnicotinamide

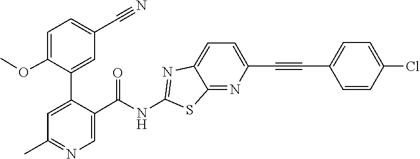 [SMILES: COc1ccc(C#N)cc1-c1cc(C)ncc1C(=O)Nc1nc2ccc(C#Cc3ccc(Cl)cc3)nc2s1]

The reaction mixture of N-(5-bromothiazolo[5,4-b]pyridin-2-yl)-4-(5-cyano-2-methoxyphenyl)-6-methylnicotinamide (0.2 g, 0.42 mmol), 1-chloro-4-ethynylbenzene (0.138 g, 1 mmol), Et₃N (0.13 g, 1.26 mmol), Pd(PPh₃)₂Cl₂ (15 mg, 0.021 mmol) and CuI (12 mg, 0.063 mmol) in acetonitrile (5 mL) under nitrogen was stirred at 80° C. for 14 h. The mixture was filtered and the filtrate was evaporated. The residue was purified by column chromatography, eluting with ethyl acetate in petroleum ether (0-100%, v/v) to give the title compound (0.13 g, 58%). LC-MS (M+H)⁺=536.4.

### Step 3: N-(5-(4-chlorophenethyl)thiazolo[5,4-b]pyridin-2-yl)-4-(5-cyano-2-methoxyphenyl)-6-methylnicotinamide

The reaction mixture of N-(5-((4-chlorophenyl)ethynyl)thiazolo[5,4-b]pyridin-2-yl)-4-(5-cyano-2-methoxyphenyl)-6-methylnicotinamide (0.13 g, 0.24 mmol), 10 wt. % Pd/C (26 mg, 20%) in THF (5 mL) under H₂ was stirred at room temperature for 14 h. The mixture was filtered and the filtrate was evaporated. The residue was purified by Prep-HPLC to give the title compound (40 mg, 31%). 1H NMR (400 MHz, d6-DMSO) δ 12.86 (s, 1H), 8.78 (s, 1H), 8.00 (d, J=8.3 Hz, 1H), 7.96-7.81 (m, 2H), 7.42 (s, 1H), 7.31 (m, 5H), 7.17 (d, J=8.6 Hz, 1H), 3.59 (s, 3H), 3.13 (dd, J=9.0, 5.9 Hz, 2H), 3.03 (dd, J=9.2, 6.1 Hz, 2H), 2.59 (s, 3H). LC-MS (M+H)⁺=540.3.

### Example 108: N-(5-(4-(ethylcarbamoyl)phenyl)thiazolo[5,4-b]pyridin-2-yl)-5-(2-methoxyphenyl)pyridazine-4-carboxamide

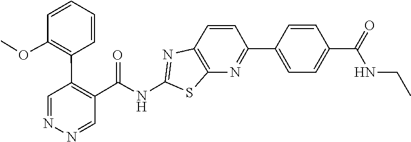 [SMILES: CCNC(=O)c1ccc(-c2ccc3nc(NC(=O)c4cnncc4-c4ccccc4OC)sc3n2)cc1]

The title compound (60 mg, 56%) was prepared in a manner similar to that in Example 73 step 5 from 4-(2-(5-(2-methoxyphenyl)pyridazine-4-carboxamido)thiazolo[5,4-b]pyridin-5-yl)benzoic acid and ethylamine. 1H NMR (400 MHz, d6-DMSO) δ 13.30 (s, 1H), 9.50 (s, 1H), 9.44 (s, 1H), 8.56 (t, J=5.5 Hz, 1H), 8.31-8.15 (m, 4H), 7.98 (d, J=8.4 Hz, 2H), 7.55 (d, J=7.5 Hz, 1H), 7.53-7.46 (m, 1H), 7.19-7.13 (m, 1H), 7.07 (d, J=8.3 Hz, 1H), 3.56 (s, 3H), 3.39-3.26 (m, 2H), 1.15 (t, J=7.2 Hz, 3H). LC-MS (M+H)⁺=511.4.

### Example 109: 4-(5-cyano-2-methoxyphenyl)-N-(5-(4-cyanophenyl)thiazolo[5,4-b]pyridin-2-yl)-6-(hydroxymethyl)nicotinamide

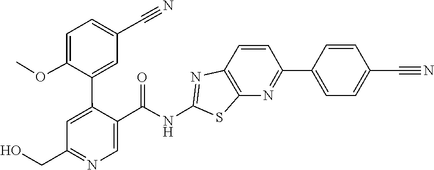 [SMILES: COc1ccc(C#N)cc1-c1cc(CO)ncc1C(=O)Nc1nc2ccc(-c3ccc(C#N)cc3)nc2s1]

### Step 1: benzyl 4-(5-cyano-2-methoxyphenyl)-6-methylnicotinate

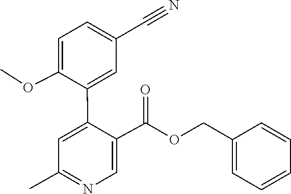 [SMILES: COc1ccc(C#N)cc1-c1cc(C)ncc1C(=O)OCc1ccccc1]

The mixture of 4-(5-cyano-2-methoxyphenyl)-6-methylnicotinic acid (0.59 g, 2.2 mmol), BnBr (0.45 g, 2.64 mmol) and K₂CO₃ (0.76 g, 5.5 mmol) in N,N-dimethylformamide (15 mL) were stirred at room temperature for 2 hours. The mixture was diluted with ethyl acetate and washed by brine, the organic phase was dried and evaporated. The residue was purified by column chromatography, eluting with ethyl acetate in petroleum ether (25%, v/v) to give the title compound (0.78 g, 99%). LC-MS (M+H)⁺=359.3.

### Step 2: 5-((benzyloxy)carbonyl)-4-(5-cyano-2-methoxyphenyl)-2-methylpyridine 1-oxide

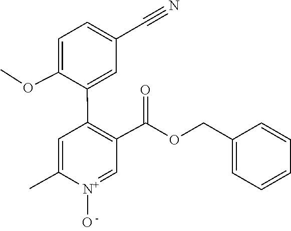 [SMILES: COc1ccc(C#N)cc1-c1cc(C)[n+](O)cc1C(=O)OCc1ccccc1]

3-Chloroperbenzoic acid (0.56 g, 3.27 mmol) was added to a solution of benzyl 4-(5-cyano-2-methoxyphenyl)-6-methylnicotinate (0.78 g, 2.18 mmol) in dichloromethane and stirred at room temperature for 14 h. The mixture was diluted with ethyl acetate and washed by brine, the organic phase was dried and evaporated. The residue was purified by column chromatography (EA) to give the title compound (0.48 g, 59%). L LC-MS (M+H)⁺=375.4.

### Step 3: benzyl 6-(acetoxymethyl)-4-(5-cyano-2-methoxyphenyl)nicotinate

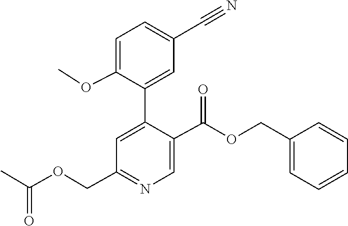 [SMILES: COc1ccc(C#N)cc1-c1cc(COC(C)=O)ncc1C(=O)OCc1ccccc1]

Acetic anhydride (0.26 g, 2.57 mmol) was added to a solution of 5-((benzyloxy)carbonyl)-4-(5-cyano-2-methoxyphenyl)-2-methylpyridine 1-oxide (0.48 g, 1.28 mmol) in acetonitrile (8 mL) under nitrogen was stirred at 80° C. for 14 h. The mixture was evaporated and the residue was purified by column chromatography eluting with ethyl acetate in petroleum ether (0-100%, v/v) to give the title compound (0.29 g, 55%). LC-MS (M+H)⁺=417.3.

### Step 4: 6-(acetoxymethyl)-4-(5-cyano-2-methoxyphenyl)nicotinic acid

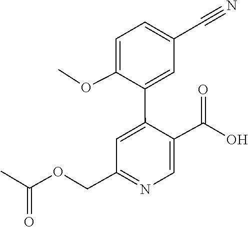 [SMILES: COc1ccc(C#N)cc1-c1cc(COC(C)=O)ncc1C(=O)O]

The title compound (0.2 g, 87.6%) was prepared in a manner similar to Example 2 of Step 3 from benzyl 6-(acetoxymethyl)-4-(5-cyano-2-methoxyphenyl)nicotinate and 10 wt. % Pd/C. LC-MS (M+H)⁺=327.2.

### Step 5: (4-(5-cyano-2-methoxyphenyl)-5-((5-(4-cyanophenyl)thiazolo[5,4-b]pyridin-2-yl)carbamoyl)pyridin-2-yl)methyl acetate

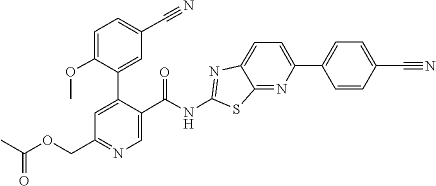 [SMILES: COc1ccc(C#N)cc1-c1cc(COC(C)=O)ncc1C(=O)Nc1nc2ccc(-c3ccc(C#N)cc3)nc2s1]

The title compound (60 mg, 35%) was prepared in a manner similar to Example 11 of Step 3 from 6-(acetoxymethyl)-4-(5-cyano-2-methoxyphenyl)nicotinic acid and 4-(2-aminothiazolo[5,4-b]pyridin-5-yl)benzonitrile. LC-MS (M+H)⁺=561.3.

### Step 6: 4-(5-cyano-2-methoxyphenyl)-N-(5-(4-cyanophenyl)thiazolo[5,4-b]pyridin-2-yl)-6-(hydroxymethyl)nicotinamide

The title compound (20 mg, 33%) was prepared in a manner similar to Example 1 of Step 3 from (4-(5-cyano-2-methoxyphenyl)-5-((5-(4-cyanophenyl)thiazolo[5,4-b]pyridin-2-yl)carbamoyl)pyridin-2-yl)methyl acetate and LiOH. 1H NMR (400 MHz, d6-DMSO) δ 13.10 (s, 1H), 8.86 (s, 1H), 8.34 (d, J=8.4 Hz, 2H), 8.24 (q, J=8.6 Hz, 2H), 8.01-7.86 (m, 4H), 7.55 (s, 1H), 7.20 (d, J=8.7 Hz, 1H), 5.60 (t, J=5.9 Hz, 1H), 4.69 (d, J=5.7 Hz, 2H), 3.61 (s, 3H). LC-MS (M+H)⁺=519.3.

### Example 110: N-(5-(4-carbamoyl-3-methylphenyl)thiazolo[5,4-b]pyridin-2-yl)-4-(5-cyano-2-methoxyphenyl)-6-methylnicotinamide

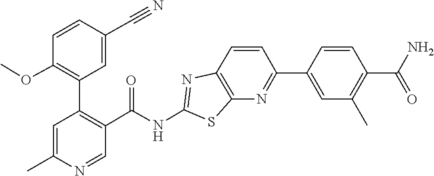 [SMILES: COc1ccc(C#N)cc1-c1cc(C)ncc1C(=O)Nc1nc2ccc(-c3ccc(C(N)=O)c(C)c3)nc2s1]

### Step 1: 4-bromo-2-methylbenzamide

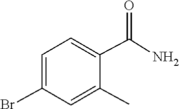

Oxalyl chloride (1.4 g, 11 mmol) was added dropwise to a solution of 4-bromo-2-methylbenzoic acid (2.15 g, 10 mmol) in dichloromethane at 0° C., it was stirred at 0° C. for 0.5 hour, then 7 N ammonia solution in methanol (2 mL) was added dropwise and stirred at room temperature for another 1 hour. The reaction was evaporated and the residue was purified by column chromatography eluting with ethyl acetate in petroleum ether (0-100%, v/v) to give the title compound (1.78 g, 84%). LC-MS (M+H)⁺=214.3.

### Step 2: 2-methyl-4-(4,4,5,5-tetramethyl-1,3,2-dioxaborolan-2-yl)benzamide

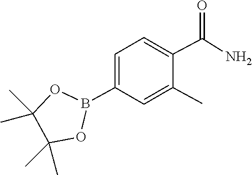 [SMILES: Cc1cc(B2OC(C)(C)C(C)(C)O2)ccc1C(N)=O]

The reaction mixture of 4-bromo-2-methylbenzamide (1.78 g, 8.36 mmol), bis(pinacolato)diboron (2.33 g, 9.16 mmol), 1,1′-bis(diphenylphosphino)ferrocene]dichloropalladium(II) (0.68 g, 0.836 mmol) and KOAc (2.46 g, 25 mmol) in 1,2-dimethoxyethane (20 mL) under nitrogen was stirred at 80° C. for 14 h. The mixture was filtered and the filtrate was evaporated. The residue was purified by column chromatography eluting with ethyl acetate in petroleum ether (0-100%, v/v) to give the title compound (2.1 g, 96%). LC-MS (M+H)⁺=262.3.

### Step 3: tert-butyl (5-(4-carbamoyl-3-methylphenyl)thiazolo[5,4-b]pyridin-2-yl)carbamate

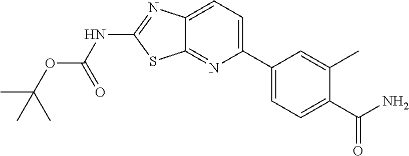 [SMILES: Cc1cc(-c2ccc3nc(NC(=O)OC(C)(C)C)sc3n2)ccc1C(N)=O]

The reaction mixture of 2-methyl-4-(4,4,5,5-tetramethyl-1,3,2-dioxaborolan-2-yl)benzamide (0.31 g, 1.2 mmol), tert-butyl (5-bromothiazolo[5,4-b]pyridin-2-yl)carbamate (0.21 g, 1 mmol), 1,1′-bis(diphenylphosphino)ferrocene]dichloropalladium(II) (82 mg, 0.1 mmol) and K₂CO₃ (0.35 g, 2.5 mmol) in dioxane (5 mL) under nitrogen was stirred at 100° C. for 14 h. The mixture was filtered and the filtrate was evaporated. The residue was purified by column chromatography eluting with ethyl acetate in petroleum ether (0-100%, v/v) to give the title compound (0.23 g, 60%). LC-MS (M+H)⁺=385.4.

### Step 4: 4-(2-aminothiazolo[5,4-b]pyridin-5-yl)-2-methylbenzamide

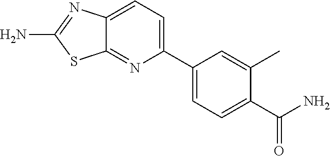 [SMILES: Cc1cc(-c2ccc3nc(N)sc3n2)ccc1C(N)=O]

Trifluoroacetic acid (2 mL) was added to a solution of tert-butyl (5-(4-carbamoyl-3-methylphenyl)thiazolo[5,4-b]pyridin-2-yl)carbamate (0.23 g, 0.6 mmol) in dichloromethane (6 mL). It was stirred at room temperature for 14 h. The solvent was evaporated and the residue was treated with a saturated aqueous solution of NaHCO₃. The crude product was purified by column chromatography eluting with ethyl acetate in petroleum ether (0-100%, v/v) to give the title compound (0.15 g, 65%). LC-MS (M+H)⁺=285.2.

### Step 5: N-(5-(4-carbamoyl-3-methylphenyl)thiazolo[5,4-b]pyridin-2-yl)-4-(5-cyano-2-methoxyphenyl)-6-methylnicotinamide

The title compound (30 mg, 50%) was prepared in a manner similar to Example 25 step 3 from 4-(2-aminothiazolo[5,4-b]pyridin-5-yl)-2-methylbenzamide and 4-(5-cyano-2-methoxyphenyl)-6-methylnicotinic acid. 1H NMR (400 MHz, d6-DMSO) δ 12.98 (s, 1H), 8.81 (s, 1H), 8.18 (d, J=8.5 Hz, 1H), 8.10 (d, J=8.6 Hz, 1H), 8.04-7.87 (m, 4H), 7.77 (s, 1H), 7.49 (d, J=8.0 Hz, 1H), 7.41 (d, J=16.1 Hz, 2H), 7.18 (d, J=8.5 Hz, 1H), 3.60 (s, 3H), 2.60 (s, 3H), 2.47 (s, 3H). LC-MS (M+H)⁺=535.3.

### Example 111: N-(5-(4-(2-hydroxyacetamido)phenyl)thiazolo[5,4-b]pyridin-2-yl)-5-(2-methoxyphenyl)pyridazine-4-carboxamide

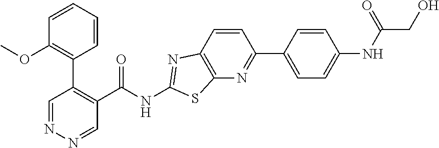 [SMILES: COc1ccccc1-c1cnncc1C(=O)Nc1nc2ccc(-c3ccc(NC(=O)CO)cc3)nc2s1]

### Step 1: 2-oxo-2-((4-(4,4,5,5-tetramethyl-1,3,2-dioxaborolan-2-yl)phenyl)amino)ethyl acetate

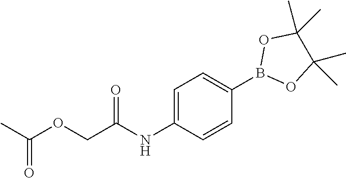 [SMILES: CC(=O)OCC(=O)Nc1ccc(B2OC(C)(C)C(C)(C)O2)cc1]

The title compound (634 mg, 71%) was prepared in a manner similar to that in Example 28 step 2 from 2-((4-bromophenyl)amino)-2-oxoethyl acetate. LC-MS (M+H)⁺=320.1.

### Step 2: N-(5-(4-(2-hydroxyacetamido)phenyl)thiazolo[5,4-b]pyridin-2-yl)-5-(2-methoxyphenyl)pyridazine-4-carboxamide

The title compound (21 mg, 31%) was prepared in a manner similar to that in Example 78 step 3 from N-(5-bromothiazolo[5,4-b]pyridin-2-yl)-5-(2-methoxyphenyl)pyridazine-4-carboxamide and 2-oxo-2-((4-(4,4,5,5-tetramethyl-1,3,2-dioxaborolan-2-yl)phenyl)amino)ethyl acetate. 1H NMR (400 MHz, d6-DMSO) δ 13.24 (brs, 1H), 9.83 (s, 1H), 9.49 (s, 1H), 9.40 (s, 1H), 8.19-8.01 (m, 4H), 7.85 (d, J=8.6 Hz, 2H), 7.56-7.40 (m, 2H), 7.18-7.10 (m, 1H), 7.07 (d, J=8.3 Hz, 1H), 5.67 (t, J=6.0 Hz, 1H), 4.03 (d, J=5.8 Hz, 2H), 3.57 (s, 3H). LC-MS (M+H)⁺=513.0.

### Example 112: N-(2-hydroxyethyl)-2-(5-(2-methoxyphenyl)pyridazine-4-carboxamido)thiazolo[5,4-b]pyridine-5-carboxamide

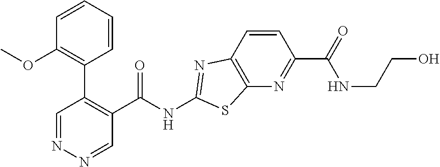 [SMILES: COc1ccccc1-c1cnncc1C(=O)Nc1nc2ccc(C(=O)NCCO)nc2s1]

The title compound (15 mg, 34%) was prepared in a manner similar to that in Example 73 step 5 from 2-(5-(2-methoxyphenyl)pyridazine-4-carboxamido)thiazolo[5,4-b]pyridine-5-carboxylic acid and 2-aminoethan-1-ol. 1H NMR (400 MHz, d6-DMSO) δ 13.47 (brs, 1H), 9.48 (s, 1H), 9.34 (s, 1H), 8.70 (t, J=5.9 Hz, 1H), 8.17-7.98 (m, 2H), 7.52-7.42 (m, 2H), 7.16-6.95 (m, 2H), 4.80 (s, 1H), 3.57-3.52 (m, 4H), 3.44-3.35 (m, 3H). LC-MS (M+H)⁺=451.0.

### Example 113: 2-(5-(2-methoxyphenyl)pyridazine-4-carboxamido)-N-(tetrahydro-2H-pyran-4-yl)thiazolo[5,4-b]pyridine-5-carboxamide

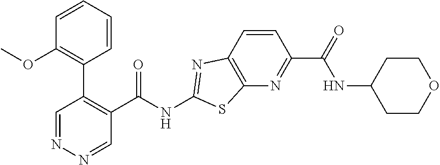 [SMILES: COc1ccccc1-c1cnncc1C(=O)Nc1nc2ccc(C(=O)NC3CCOCC3)nc2s1]

The title compound (47 mg, 77%) was prepared in a manner similar to that in Example 73 step 5 from 2-(5-(2-methoxyphenyl)pyridazine-4-carboxamido)thiazolo[5,4-b]pyridine-5-carboxylic acid and tetrahydro-2H-pyran-4-amine. 1H NMR (300 MHz, d6-DMSO) δ 13.46 (brs, 1H), 9.50 (s, 1H), 9.42 (s, 1H), 8.78 (d, J=8.4 Hz, 1H), 8.28-8.10 (m, 2H), 7.59-7.43 (m, 2H), 7.20-7.02 (m, 2H), 4.12-3.99 (m, 1H), 3.95-3.83 (m, 2H), 3.55 (s, 3H), 3.47-3.35 (m, 2H), 1.84-1.67 (m, 4H). LC-MS (M+H)⁺=491.0.

### Example 114: N-(5-(cyclopentyloxy)thiazolo[5,4-b]pyridin-2-yl)-5-(2-methoxyphenyl)pyridazine-4-carboxamide

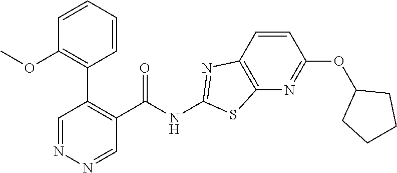 [SMILES: COc1ccccc1-c1cnncc1C(=O)Nc1nc2ccc(OC3CCCC3)nc2s1]

### Step 1: 5-(cyclopentyloxy)thiazolo[5,4-b]pyridin-2-amine

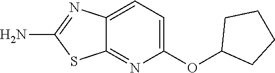 [SMILES: Nc1nc2ccc(OC3CCCC3)nc2s1]

The title compound (90 mg, 43%) was prepared in a manner similar to that in Example 95 step 2 from 2-aminothiazolo[5,4-b]pyridin-5-ol and iodocyclopentane. LC-MS (M+H)⁺=236.0.

### Step 2: N-(5-(cyclopentyloxy)thiazolo[5,4-b]pyridin-2-yl)-5-(2-methoxyphenyl)pyridazine-4-carboxamide

The title compound (9 mg, 7%) was prepared in a manner similar to that in Example 81 step 2 from 5-(cyclopentyloxy)thiazolo[5,4-b]pyridin-2-amine and 5-(2-methoxyphenyl)pyridazine-4-carboxylic acid. 1H NMR (300 MHz, d6-DMSO) δ 9.42 (s, 1H), 9.12 (s, 1H), 7.74-7.65 (m, 1H), 7.45-7.30 (m, 2H), 7.10-6.96 (m, 2H), 6.67-6.58 (m, 1H), 5.40-5.28 (m, 1H), 3.60 (s, 3H), 1.98-1.86 (m, 2H), 1.76-1.53 (m, 6H). LC-MS (M+H)⁺=448.0.

### Example 115: N-(5-(4-(acetamidomethyl)phenyl)thiazolo[5,4-b]pyridin-2-yl)-4-(5-cyano-2-methoxyphenyl)-6-methylnicotinamide

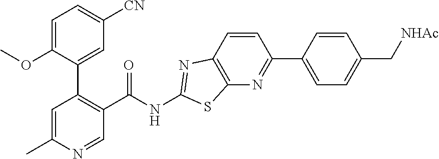 [SMILES: COc1ccc(C#N)cc1-c1cc(C)ncc1C(=O)Nc1nc2ccc(-c3ccc(CNC(C)=O)cc3)nc2s1]

The title compound (23 mg, 22%) was prepared in a manner similar to Example 96 step 2 from N-(5-bromothiazolo[5,4-b]pyridin-2-yl)-4-(5-cyano-2-methoxyphenyl)-6-methylnicotinamide and (4-(acetamidomethyl)phenyl)boronic acid. ¹H NMR (400 MHz, d6-DMSO) δ 12.96 (s, 1H), 8.81 (s, 1H), 8.39 (t, J=6.0 Hz, 1H), 8.16 (d, J=8.7 Hz, 1H), 8.06 (dd, J=11.9, 8.5 Hz, 3H), 7.94-7.85 (m, 2H), 7.43 (s, 1H), 7.38 (d, J=8.3 Hz, 2H), 7.18 (d, J=8.6 Hz, 1H), 4.31 (d, J=5.9 Hz, 2H), 3.60 (s, 3H), 2.60 (s, 3H), 1.90 (s, 3H). LC-MS (M+H)⁺=549.1.

### Example 116: 4-(5-cyano-2-methoxyphenyl)-N-(5-(4-(cyanomethyl)phenyl)thiazolo[5,4-b]pyridin-2-yl)-6-methylnicotinamide

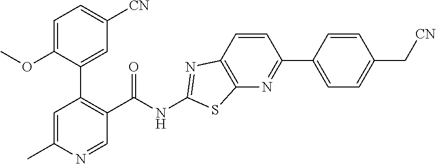 [SMILES: COc1ccc(C#N)cc1-c1cc(C)ncc1C(=O)Nc1nc2ccc(-c3ccc(CC#N)cc3)nc2s1]

The title compound (23 mg, 22%) was prepared in a manner similar to Example 96 step 2 from N-(5-bromothiazolo[5,4-b]pyridin-2-yl)-4-(5-cyano-2-methoxyphenyl)-6-methylnicotinamide and (4-(cyanomethyl)phenyl)boronic acid. ¹H NMR (400 MHz, d6-DMSO) δ 12.98 (s, 1H), 8.80 (s, 1H), 8.18 (dd, J=11.3, 8.5 Hz, 3H), 8.09 (d, J=8.6 Hz, 1H), 7.95-7.89 (m, 2H), 7.49 (d, J=8.3 Hz, 2H), 7.44 (s, 1H), 7.18 (d, J=9.2 Hz, 1H), 4.12 (s, 2H), 3.60 (s, 3H), 2.60 (s, 3H). LC-MS (M+H)⁺=517.1.

### Example 117: N-(5-(2,4-dichlorophenyl)thiazolo[5,4-b]pyridin-2-yl)-5-(2-methoxyphenyl)pyridazine-4-carboxamide

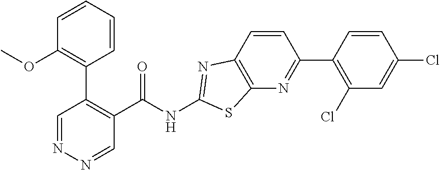 [SMILES: COc1ccccc1-c1cnncc1C(=O)Nc1nc2ccc(-c3ccc(Cl)cc3Cl)nc2s1]

### Step 1: tert-butyl (5-(2,4-dichlorophenyl)thiazolo[5,4-b]pyridin-2-yl)carbamate

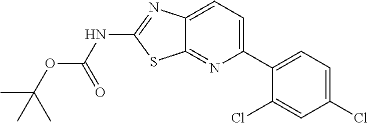 [SMILES: CC(C)(C)OC(=O)Nc1nc2ccc(-c3ccc(Cl)cc3Cl)nc2s1]

The title compound (150 mg, 81%) was prepared in a manner similar to that in Example 78 step 3 from tert-butyl (5-bromothiazolo[5,4-b]pyridin-2-yl)carbamate and (2,4-dichlorophenyl)boronic acid. LC-MS (M+H)⁺=396.1.

### Step 2: 5-(2,4-dichlorophenyl)thiazolo[5,4-b]pyridin-2-amine

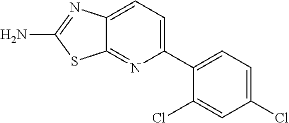 [SMILES: Nc1nc2ccc(-c3ccc(Cl)cc3Cl)nc2s1]

The title compound (139 mg, 86%) was prepared in a manner similar to that in Example 1 step 6 from tert-butyl (5-(2,4-dichlorophenyl)thiazolo[5,4-b]pyridin-2-yl)carbamate. LC-MS (M+H)⁺=296.1.

### Step 3: N-(5-(2,4-dichlorophenyl)thiazolo[5,4-b]pyridin-2-yl)-5-(2-methoxyphenyl)pyridazine-4-carboxamide

The title compound (28 mg, 12%) was prepared in a manner similar to that in Example 11 step 3 from 5-(2-methoxyphenyl)pyridazine-4-carboxylic acid and 5-(2,4-dichlorophenyl)thiazolo[5,4-b]pyridin-2-amine. 1H NMR (400 MHz, d6-DMSO) δ 13.34 (s, 1H), 9.50 (s, 1H), 9.40 (s, 1H), 8.21 (d, J=7.7 Hz, 1H), 7.82-7.74 (m, 2H), 7.73-7.66 (m, 1H), 7.61-7.43 (m, 3H), 7.14 (t, J=7.6 Hz, 1H), 7.07 (d, J=8.3 Hz, 1H), 3.56 (s, 3H). LC-MS (M+H)⁺=508.3.

### Example 118: 5-(2-methoxyphenyl)-N-(5-((tetrahydrofuran-2-yl)methoxy)thiazolo[5,4-b]pyridin-2-yl)pyridazine-4-carboxamide

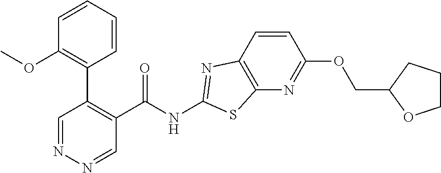 [SMILES: COc1ccccc1-c1cnncc1C(=O)Nc1nc2ccc(OCC3CCCO3)nc2s1]

N-(5-bromothiazolo[5,4-b]pyridin-2-yl)-5-(2-methoxyphenyl)pyridazine-4-carboxamide (69.3 mg, 0.678 mmole) was dissolved in dry THF (5 mL). NaH (22.6 mg, 0.6565 mmole) was added at 0° C. under nitrogen atmosphere. (Tetrahydrofuran-2-yl)methanol (100 mg, 0.226 mmole) and CuI (200 mg, 1.05 mmole) were added and heated to 80° C. for 14 h under nitrogen atmosphere. Water (10 mL) was added and extracted with ethyl acetate. The combine organic layers were washed with brine and dried over Na₂SO₄. The title compound (4 mg, 3.8%) was obtained by purification over silica gel chromatography and pre-HPLC. 1H NMR (400 MHz, d6-DMSO) δ 13.06 (s, 1H), 9.46 (s, 1H), 9.41 (s, 1H), 8.09 (d, J=8.8 Hz, 1H), 7.61-7.40 (m, 2H), 7.14 (t, J=7.4 Hz, 1H), 7.06 (d, J=8.2 Hz, 1H), 6.95 (d, J=8.8 Hz, 1H), 4.24 (tdd, J=10.6, 8.8, 3.9 Hz, 3H), 3.79 (dd, J=14.7, 6.8 Hz, 1H), 3.67 (dd, J=13.9, 7.6 Hz, 1H), 3.54 (s, 3H), 2.00 (d, J=8.3 Hz, 1H), 1.94-1.77 (m, 2H), 1.74-1.61 (m, 1H). LC-MS (M+H)⁺=464.0.

### Example 119: N-(5-(4-cyano-2-methoxyphenyl)thiazolo[5,4-b]pyridin-2-yl)-5-(2-methoxyphenyl)pyridazine-4-carboxamide

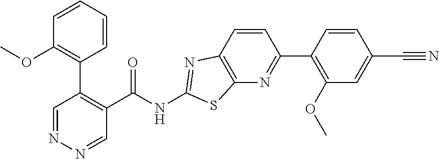 [SMILES: COc1cc(C#N)ccc1-c1ccc2nc(NC(=O)c3cnncc3-c3ccccc3OC)sc2n1]

### Step 1: tert-butyl (5-(4-cyano-2-methoxyphenyl)thiazolo[5,4-b]pyridin-2-yl)carbamate

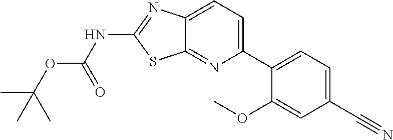 [SMILES: COc1cc(C#N)ccc1-c1ccc2nc(NC(=O)OC(C)(C)C)sc2n1]

The title compound (348 mg, 99%) was prepared in a manner similar to that in Example 78 step 3 from tert-butyl (5-bromothiazolo[5,4-b]pyridin-2-yl)carbamate and 3-methoxy-4-(4,4,5,5-tetramethyl-1,3,2-dioxaborolan-2-yl)benzonitrile. LC-MS (M+H)⁺=383.0.

### Step 2: 4-(2-aminothiazolo[5,4-b]pyridin-5-yl)-3-methoxybenzonitrile

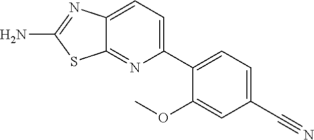 [SMILES: COc1cc(C#N)ccc1-c1ccc2nc(N)sc2n1]

The title compound (101 mg, 39%) was prepared in a manner similar to that in Example 1 step 6 from tert-butyl (5-(4-cyano-2-methoxyphenyl)thiazolo[5,4-b]pyridin-2-yl)carbamate. LC-MS (M+H)⁺=283.0.

### Step 3: N-(5-(4-cyano-2-methoxyphenyl)thiazolo[5,4-b]pyridin-2-yl)-5-(2-methoxyphenyl)pyridazine-4-carboxamide

The title compound (60 mg, 22%) was prepared in a manner similar to that described in Example 11 step 3 from 4-(2-aminothiazolo[5,4-b]pyridin-5-yl)-3-methoxybenzonitrile and 5-(2-methoxyphenyl)pyridazine-4-carboxylic acid. 1H NMR (400 MHz, d6-DMSO)δ 13.32 (s, 1H), 9.50 (s, 1H), 9.43 (s, 1H), 8.21 (d, J=8.6 Hz, 1H), 8.03 (d, J=8.5 Hz, 1H), 7.96 (d, J=7.9 Hz, 1H), 7.69 (d, J=1.1 Hz, 1H), 7.59-7.53 (m, 2H), 7.52-7.45 (m, 1H), 7.15 (t, J=7.5 Hz, 1H), 7.07 (d, J=8.3 Hz, 1H), 3.93 (s, 3H), 3.56 (s, 3H). LC-MS (M+H)⁺=495.0.

### Example 120: N-(5-(4-cyanophenyl)thiazolo[5,4-b]pyridin-2-yl)-1,2,6′-trimethyl-6-oxo-1,6-dihydro-[3,4′-bipyridine]-3′-carboxamide

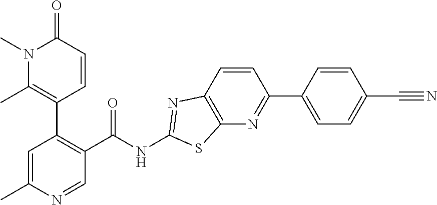 [SMILES: Cc1cc(-c2ccc(=O)n(C)c2C)c(C(=O)Nc2nc3ccc(-c4ccc(C#N)cc4)nc3s2)cn1]

### Step 1: 5-bromo-1,6-dimethylpyridin-2(1H)-one

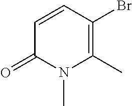

The title compound (651 mg, 59%) was prepared in a manner similar to that described in Example 87 step 1 from 5-bromo-6-methylpyridin-2(1H)-one and iodomethane. LC-MS (M+H)⁺=202.0.

### Step 2: 1,6-dimethyl-5-(4,4,5,5-tetramethyl-1,3,2-dioxaborolan-2-yl)pyridin-2(1H)-one

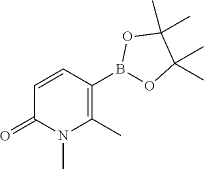 [SMILES: Cc1c(B2OC(C)(C)C(C)(C)O2)ccc(=O)n1C]

The title compound (398 mg, 64%) was prepared in a manner similar to that described in Example 28 step 2 from 5-bromo-1,6-dimethylpyridin-2(1H)-one. LC-MS (M+H)⁺=250.1.

### Step 3: methyl 1,2,6′-trimethyl-6-oxo-1,6-dihydro-[3,4′-bipyridine]-3′-carboxylate

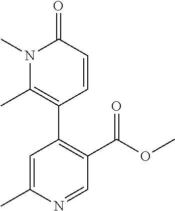

The title compound (243 mg, 92%) was prepared in a manner similar to that described in Example 2 step 2 from methyl 4-chloro-6-methylnicotinate and 1,6-dimethyl-5-(4,4,5,5-tetramethyl-1,3,2-dioxaborolan-2-yl)pyridin-2(1H)-one. LC-MS (M+H)⁺=273.1.

### Step 4: 1,2,6′-trimethyl-6-oxo-1,6-dihydro-[3,4′-bipyridine]-3′-carboxylic acid

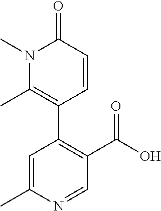 [SMILES: Cc1cc(-c2ccc(=O)n(C)c2C)c(C(=O)O)cn1]

The title compound (110 mg, 88%) was prepared in a manner similar to that described in Example 1 step 3 from methyl 1,2,6′-trimethyl-6-oxo-1,6-dihydro-[3,4′-bipyridine]-3′-carboxylate. LC-MS (M+H)⁺=259.1.

### Step 5: N-(5-(4-cyanophenyl)thiazolo[5,4-b]pyridin-2-yl)-1,2,6′-trimethyl-6-oxo-1,6-dihydro-[3,4′-bipyridine]-3′-carboxamide

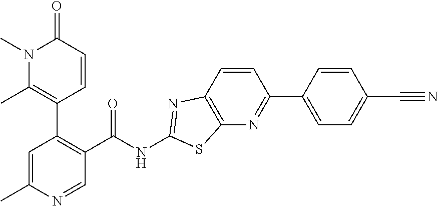 [SMILES: Cc1cc(-c2ccc(=O)n(C)c2C)c(C(=O)Nc2nc3ccc(-c4ccc(C#N)cc4)nc3s2)cn1]

The title compound (21.5 mg, 12.5%) was prepared in a manner similar to that described in Example 11 step 3 from 1,2,6′-trimethyl-6-oxo-1,6-dihydro-[3,4′-bipyridine]-3′-carboxylic acid and 4-(2-aminothiazolo[5,4-b]pyridin-5-yl)benzonitrile. ¹H NMR (400 MHz, d6-DMSO) δ: 13.15 (s, 1H), 8.85 (s, 1H), 8.38-8.30 (m, 2H), 8.26-8.18 (m, 2H), 8.00-7.90 (m, 2H), 7.29 (s, 1H), 7.26-7.18 (s, 1H), 6.38-6.30 (m, 1H), 3.47 (s, 3H), 2.51 (s, 3H), 2.20 (s, 3H). LC-MS (M+H)⁺=493.1.

### Example 121: N-(5-(4-cyano-2-methylphenyl)thiazolo[5,4-b]pyridin-2-yl)-5-(2-methoxyphenyl)pyridazine-4-carboxamide

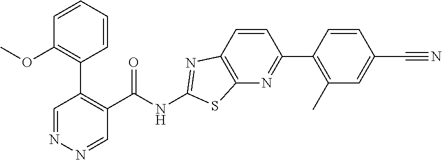 [SMILES: COc1ccccc1-c1cnncc1C(=O)Nc1nc2ccc(-c3ccc(C#N)cc3C)nc2s1]

The title compound (17 mg, 16%) was prepared in a manner similar to that described in Example 78 step 3 from N-(5-bromothiazolo[5,4-b]pyridin-2-yl)-5-(2-methoxyphenyl)pyridazine-4-carboxamide and (4-cyano-2-methylphenyl)boronic acid. ¹H NMR (400 MHz, d6-DMSO)δ 13.34 (s, 1H), 9.50 (s, 1H), 9.44 (s, 1H), 8.27 (d, J=8.0 Hz, 1H), 7.86 (s, 1H), 7.79 (d, J=7.8 Hz, 1H), 7.74 (d, J=8.3 Hz, 1H), 7.67 (d, J=8.0 Hz, 1H), 7.55 (d, J=8.4 Hz, 1H), 7.49 (t, J=7.6 Hz, 1H), 7.16 (t, J=7.5 Hz, 1H), 7.07 (d, J=8.4 Hz, 1H), 3.56 (s, 3H), 2.41 (s, 3H). LC-MS (M+H)⁺=479.0.

### Example 122: 4-(5-cyano-2-methoxyphenyl)-N-(5-(4-(2-hydroxyethoxy)phenyl)thiazolo[5,4-b]pyridin-2-yl)-6-methylnicotinamide

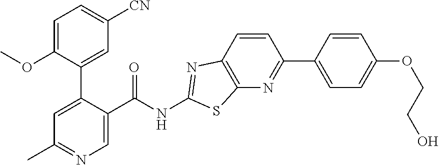 [SMILES: COc1ccc(C#N)cc1-c1cc(C)ncc1C(=O)Nc1nc2ccc(-c3ccc(OCCO)cc3)nc2s1]

### Step 1: tert-butyldimethyl(2-(4-(4,4,5,5-tetramethyl-1,3,2-dioxaborolan-2-yl)phenoxy)ethoxy)silane

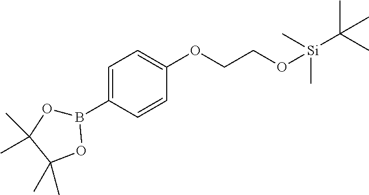 [SMILES: CC1(C)OB(c2ccc(OCCO[Si](C)(C)C(C)(C)C)cc2)OC1(C)C]

The title compound (519 mg, 50%) was prepared in a manner similar to that in Example 87 step 1 from 4-(4,4,5,5-tetramethyl-1,3,2-dioxaborolan-2-yl)phenol and (2-bromoethoxy)(tert-butyl)dimethylsilane. LC-MS (M+H)⁺=379.1.

### Step 2: N-(5-(4-(2-((tert-butyldimethylsilyl)oxy)ethoxy)phenyl)thiazolo[5,4-b]pyridin-2-yl)-4-(5-cyano-2-methoxyphenyl)-6-methylnicotinamide

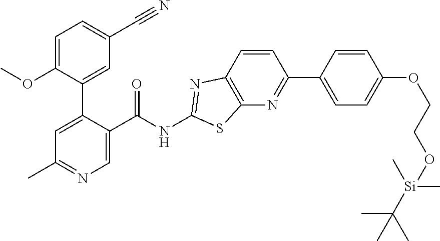

The title compound (22 mg, 79%) was prepared in a manner similar to that in Example 78 step 3 from N-(5-bromothiazolo[5,4-b]pyridin-2-yl)-4-(5-cyano-2-methoxyphenyl)-6-methylnicotinamide and tert-butyldimethyl(2-(4-(4,4,5,5-tetramethyl-1,3,2-dioxaborolan-2-yl)phenoxy)ethoxy)silane. LC-MS (M+H)⁺=652.1.

### Step 3: 4-(5-cyano-2-methoxyphenyl)-N-(5-(4-(2-hydroxyethoxy)phenyl)thiazolo[5,4-b]pyridin-2-yl)-6-methylnicotinamide

To a solution of N-(5-(4-(2-((tert-butyldimethylsilyl)oxy)ethoxy)phenyl)thiazolo[5,4-b]pyridin-2-yl)-4-(5-cyano-2-methoxyphenyl)-6-methylnicotinamide (24 mg, 0.037 mmol) in THF (1.0 mL) were added TBAF (149 uL, 0.149 mmol, 1M in THF) dropwise at room temperature. The resulting mixture was stirred for 1 h at room temperature under nitrogen atmosphere. When all starting material was consumed, the resulting mixture was concentrated under reduced pressure. The residue was purified by prep-HPLC under the following conditions: column, XBridge Shield RP18 OBD Column, 30×150 mm, 5 um; mobile phase, acetonitrile in water (with 10 mmol/L NH₄HCO₃ and 0.1% NH₄OH), 50% to 70% gradient in 10 min; detector, UV 254 nm. The title compound (6 mg, 31%) obtained. ¹H NMR (400 MHz, d6-DMSO) δ 8.80 (s, 1H), 8.16-7.96 (m, 4H), 7.94-7.87 (m, 2H), 7.43 (s, 1H), 7.22-7.15 (m, 1H), 7.11-7.04 (m, 2H), 4.07 (t, J=4.9 Hz, 2H), 3.75 (t, J=4.9 Hz, 2H), 3.62 (s, 3H), 2.61 (s, 3H). LC-MS (M+H)⁺=538.0.

### Example 123: 4-(5-cyano-2-methoxyphenyl)-N-(5-(4-(2-methoxyethoxy)phenyl)thiazolo[5,4-b]pyridin-2-yl)-6-methylnicotinamide

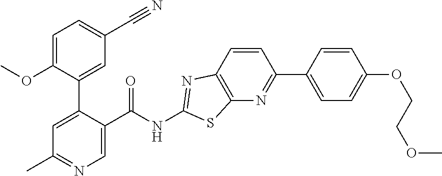

### Step 1: tert-butyl (5-(4-(2-methoxyethoxy)phenyl)thiazolo[5,4-b]pyridin-2-yl)carbamate

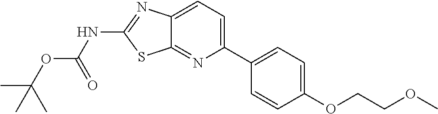 [SMILES: COCCOc1ccc(-c2ccc3nc(NC(=O)OC(C)(C)C)sc3n2)cc1]

The title compound (86 mg, 29%) was prepared in a manner similar to that in Example 78 step 3 from (4-(2-methoxyethoxy)phenyl)boronic acid and tert-butyl (5-bromothiazolo[5,4-b]pyridin-2-yl)carbamate. LC-MS (M+H)⁺=402.2

### Step 2: 5-(4-(2-methoxyethoxy)phenyl)thiazolo[5,4-b]pyridin-2-amine

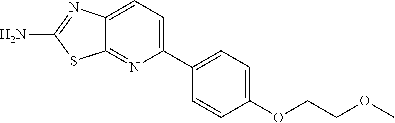 [SMILES: COCCOc1ccc(-c2ccc3nc(N)sc3n2)cc1]

The title compound (45 mg, 74%) was prepared in a manner similar to that in Example 1 step 6 from tert-butyl (5-(4-(2-methoxyethoxy)phenyl)thiazolo[5,4-b]pyridin-2-yl)carbamate. LC-MS (M+H)⁺=302.2

### Step 3: 2-bromo-5-(4-(2-methoxyethoxy)phenyl)thiazolo[5,4-b]pyridine

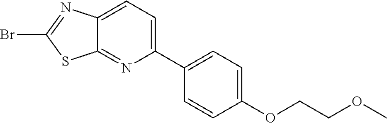 [SMILES: COCCOc1ccc(-c2ccc3nc(Br)sc3n2)cc1]

The title compound (27 mg, 44%) was prepared in a manner similar to that in Example 69 step 2 from 5-(4-(2-methoxyethoxy)phenyl)thiazolo[5,4-b]pyridin-2-amine. LC-MS (M+H)⁺=364.9

### Step 4: 4-(5-cyano-2-methoxyphenyl)-N-(5-(4-(2-methoxyethoxy)phenyl)thiazolo[5,4-b]pyridin-2-yl)-6-methylnicotinamide

The title compound (6 mg, 16%) was prepared in a manner similar to that in Example 69 step 4 from 4-(5-cyano-2-methoxyphenyl)-6-methylnicotinamide and 2-bromo-5-(4-(2-methoxyethoxy)phenyl)thiazolo[5,4-b]pyridine. 1H NMR (400 MHz, d6-DMSO) δ 12.95 (brs, 1H), 8.80 (s, 1H), 8.19-7.97 (m, 4H), 7.95-7.88 (m, 2H), 7.43 (s, 1H), 7.18 (d, J=8.4 Hz, 1H), 7.10-7.03 (m, 2H), 4.20-4.13 (m, 2H), 3.72-3.66 (m, 2H), 3.60 (s, 3H), 3.33 (s, 3H), 2.60 (s, 3H). LC-MS (M+H)⁺=552.2.

### Example 124: 5-(2-methoxyphenyl)-N-(5-(morpholine-4-carbonyl)thiazolo[5,4-b]pyridin-2-yl)pyridazine-4-carboxamide

The title compound (41 mg, 70%) was prepared in a manner similar to that in Example 73 step 5 from 2-(5-(2-methoxyphenyl)pyridazine-4-carboxamido)thiazolo[5,4-b]pyridine-5-carboxylic acid and morpholine. 1H NMR (300 MHz, d6-DMSO) δ 13.40 (brs, 1H), 9.50 (s, 1H), 9.41 (s, 1H), 8.26-8.17 (m, 1H), 7.71 (d, J=8.4 Hz, 1H), 7.57-7.43 (m, 2H), 7.19-7.02 (m, 2H), 3.71-3.65 (m, 4H), 3.37-3.31 (m, 7H). LC-MS (M+H)⁺=477.0.

### Example 125: N-(5-(7-chloro-1-methyl-1H-indazol-4-yl)thiazolo[5,4-b]pyridin-2-yl)-5-(2-methoxyphenyl)pyridazine-4-carboxamide

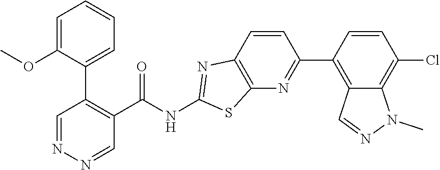 [SMILES: COc1ccccc1-c1cnncc1C(=O)Nc1nc2ccc(-c3ccc(Cl)c4c3cnn4C)nc2s1]

### Step 1: 4-bromo-7-chloro-1-methyl-1H-indazole

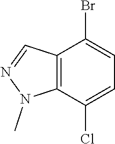

NaH (260 mg, 6.5 mmol) was added to a solution of 4-bromo-7-chloro-1H-indazole (1.0 g, 4.3 mmol) in N,N-dimethylformamide (10 mL) at 0° C. The mixture was stirred at 0° C. for 10 min. To the mixture was added Mel (1.2 g, 8.4 mmol). The mixture was stirred at r. t. for 1 h. The mixture was diluted with water (30 mL), extracted with ethyl acetate (3×30 mL). The combined organic layers were washed with water (50 mL), brine (50 mL). dried over Na₂SO₄, filtered and concentrated to give the crude title compound (1.0 g). LC-MS (M+H)⁺=245.0, 247.0.

### Step 2: 7-chloro-1-methyl-4-(4,4,5,5-tetramethyl-1,3,2-dioxaborolan-2-yl)-1H-indazole

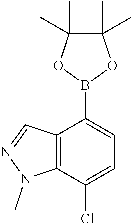 [SMILES: Cn1ncc2c(B3OC(C)(C)C(C)(C)O3)ccc(Cl)c21]

The title compound (0.5 g) was prepared in a manner similar to that in Example 28 step 2 from 4-bromo-7-chloro-1-methyl-1H-indazole and 4,4,4′,4′,5,5,5′,5′-octamethyl-2,2′-bi(1,3,2-dioxaborolane). LC-MS (M+H)⁺=293.2, 295.2.

### Step 3: N-(5-(7-chloro-1-methyl-1H-indazol-4-yl)thiazolo[5,4-b]pyridin-2-yl)-5-(2-methoxyphenyl)pyridazine-4-carboxamide

The title compound (15 mg, 2%) was prepared in a manner similar to that in Example 78 step 3 from 7-chloro-1-methyl-4-(4,4,5,5-tetramethyl-1,3,2-dioxaborolan-2-yl)-1H-indazole and N-(5-bromothiazolo[5,4-b]pyridin-2-yl)-5-(2-methoxyphenyl)pyridazine-4-carboxamide. 1H NMR (400 MHz, d6-DMSO) δ 13.34 (s, 1H), 9.51 (s, 1H), 9.43 (s, 1H), 8.74 (s, 1H), 8.27 (d, J=8.2 Hz, 1H), 8.18 (d, J=8.5 Hz, 1H), 7.75 (d, J=7.9 Hz, 1H), 7.59 (d, J=7.9 Hz, 1H), 7.55 (d, J=7.2 Hz, 1H), 7.52-7.46 (m, 1H), 7.19-7.13 (m, 1H), 7.07 (d, J=8.4 Hz, 1H), 4.39 (s, 3H), 3.57 (s, 3H). LC-MS (M+H)⁺=528.3, 530.2.

### Example 126: N-(5-(7-chloro-2-methyl-2H-indazol-4-yl)thiazolo[5,4-b]pyridin-2-yl)-5-(2-methoxyphenyl)pyridazine-4-carboxamide

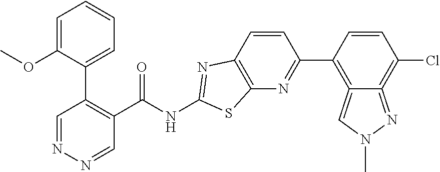

### Step 1: 4-bromo-7-chloro-2-methyl-2H-indazole

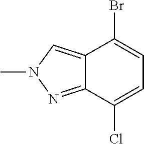

The title compound was prepared in a manner similar to that described in Example 125 step 1 from 4-bromo-7-chloro-1H-indazole. LC-MS (M+H)⁺=245.0, 247.0.

### Step 2: 7-chloro-2-methyl-4-(4,4,5,5-tetramethyl-1,3,2-dioxaborolan-2-yl)-2H-indazole

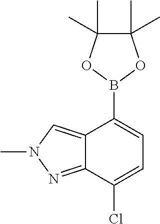

The title compound was prepared in a manner similar to that described in Example 28 step 2 from 4-bromo-7-chloro-2-methyl-2H-indazole. LC-MS (M+H)⁺=293.2, 295.2.

### Step 3: N-(5-(7-chloro-2-methyl-2H-indazol-4-yl)thiazolo[5,4-b]pyridin-2-yl)-5-(2-methoxyphenyl)pyridazine-4-carboxamide

The title compound (15 mg, 2%) was prepared in a manner similar to that described in Example 78 step 3 from 7-chloro-2-methyl-4-(4,4,5,5-tetramethyl-1,3,2-dioxaborolan-2-yl)-2H-indazole and N-(5-bromothiazolo[5,4-b]pyridin-2-yl)-5-(2-methoxyphenyl)pyridazine-4-carboxamide. ¹H NMR (400 MHz, d6-DMSO) δ 13.30 (s, 1H), 9.51 (s, 1H), 9.42 (s, 1H), 9.18 (s, 1H), 8.24-8.18 (m, 2H), 7.77 (d, J=7.6 Hz, 1H), 7.54 (d, J=7.6 Hz, 1H), 7.52-7.45 (m, 2H), 7.18-7.12 (m, 1H), 7.07 (d, J=8.2 Hz, 1H), 4.28 (s, 3H), 3.57 (s, 3H). LC-MS (M+H)⁺=528.3, 530.3.

### Example 127: 4-(5-cyano-2-methoxyphenyl)-6-methyl-N-(5-(tetrahydrofuran-3-yl)thiazolo[5,4-b]pyridin-2-yl)nicotinamide

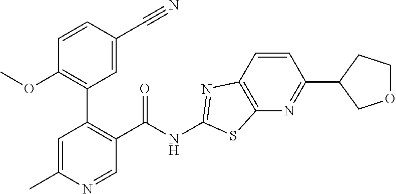 [SMILES: COc1ccc(C#N)cc1-c1cc(C)ncc1C(=O)Nc1nc2ccc(C3CCOC3)nc2s1]

### Step 1: tert-butyl (5-(2,5-dihydrofuran-3-yl)thiazolo[5,4-b]pyridin-2-yl)carbamate

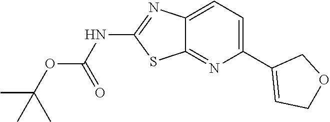 [SMILES: CC(C)(C)OC(=O)Nc1nc2ccc(C3=CCOC3)nc2s1]

The title compound (400 mg, 83%) was prepared in a manner similar to that in Example 2 step 2 from tert-butyl (5-bromothiazolo[5,4-b]pyridin-2-yl)carbamate and 2-(2,5-dihydrofuran-3-yl)-4,4,5,5-tetramethyl-1,3,2-dioxaborolane. LC-MS (M+H)⁺=320.0.

### Step 2: tert-butyl (5-(tetrahydrofuran-3-yl)thiazolo[5,4-b]pyridin-2-yl)carbamate

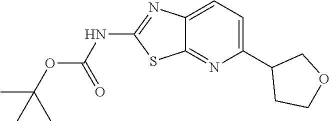 [SMILES: CC(C)(C)OC(=O)Nc1nc2ccc(C3CCOC3)nc2s1]

The title compound (179 mg, 90%) was prepared in a manner similar to that in Example 67 step 2 from tert-butyl (5-(2,5-dihydrofuran-3-yl)thiazolo[5,4-b]pyridin-2-yl)carbamate. LC-MS (M+H)⁺=322.1.

### Step 3: 5-(tetrahydrofuran-3-yl)thiazolo[54-b]pyridin-2-amine

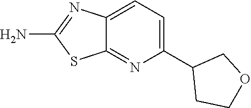 [SMILES: Nc1nc2ccc(C3CCOC3)nc2s1]

The title compound (100 mg, 90%) was prepared in a manner similar to that in Example 1 step 6 from tert-butyl (5-(tetrahydrofuran-3-yl)thiazolo[5,4-b]pyridin-2-yl)carbamate. LC-MS (M+H)⁺=222.1.

### Step 4: 4-(5-cyano-2-methoxyphenyl)-6-methyl-N-(5-(tetrahydrofuran-3-yl)thiazolo[5,4-b]pyridin-2-yl)nicotinamide

The title compound (27 mg, 34%) was prepared in a manner similar to that in Example 25 step 3 from 4-(5-cyano-2-methoxyphenyl)-6-methylnicotinic acid and 5-(tetrahydrofuran-3-yl)thiazolo[5,4-b]pyridin-2-amine. 1H NMR (400 MHz, d6-DMSO) δ: 12.88 (s, 1H), 8.78 (s, 1H), 8.04 (d, J=8.3 Hz, 1H), 7.94-7.83 (m, 2H), 7.43 (d, J=7.5 Hz, 2H), 7.16 (d, J=8.5 Hz, 1H), 4.09 (t, J=7.8 Hz, 1H), 3.95 (td, J=8.2, 4.9 Hz, 1H), 3.82 (dd, J=15.5, 7.6 Hz, 1H), 3.75 (t, J=7.7 Hz, 1H), 3.72-3.61 (m, 1H), 3.57 (s, 3H), 2.59 (s, 3H), 2.37-2.27 (m, 1H), 2.16 (dq, J=12.2, 7.8 Hz, 1H). LC-MS (M+H)⁺=472.4.

### Example 128: N-(5-(4-cyano-2-fluorophenyl)thiazolo[5,4-b]pyridin-2-yl)-5-(2-methoxyphenyl)pyridazine-4-carboxamide

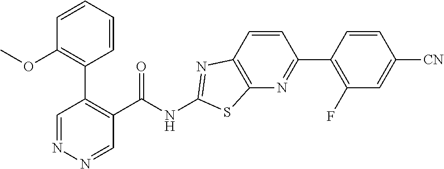 [SMILES: COc1ccccc1-c1cnncc1C(=O)Nc1nc2ccc(-c3ccc(C#N)cc3F)nc2s1]

The title compound (30 mg, 6%) was prepared in a manner similar to that in Example 78 step 3 from N-(5-bromothiazolo[5,4-b]pyridin-2-yl)-5-(2-methoxyphenyl)pyridazine-4-carboxamide and 3-fluoro-4-(4,4,5,5-tetramethyl-1,3,2-dioxaborolan-2-yl)benzonitrile. 1H NMR (400 MHz, d6-DMSO) δ 13.41 (s, 1H), 9.51 (s, 1H), 9.44 (s, 1H), 8.31 (d, J=8.7 Hz, 1H), 8.22-8.17 (m, 1H), 8.08-7.98 (m, 2H), 7.85 (d, J=8.1 Hz, 1H), 7.55 (d, J=7.6 Hz, 1H), 7.52-7.46 (m, 1H), 7.19-7.13 (m, 1H), 7.07 (d, J=8.4 Hz, 1H), 3.55 (s, 3H). LC-MS (M+H)⁺=483.4.

### Example 129: N-(5-(4-chloro-3-methylphenoxy)thiazolo[5,4-b]pyridin-2-yl)-5-(2-methoxyphenyl)pyridazine-4-carboxamide

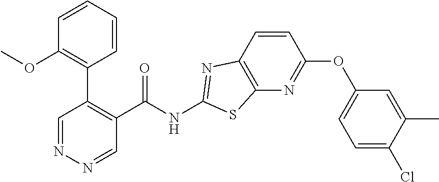

The reaction mixture of N-(5-bromothiazolo[5,4-b]pyridin-2-yl)-5-(2-methoxyphenyl)pyridazine-4-carboxamide (0.1 g, 0.23 mmol), 4-chloro-3-methylphenol (0.036 g, 0.25 mmol), K₃PO₄ (98 mg, 0.46 mmol), picolinic acid (5 mg, 0.046 mmol) and CuI (4.4 mg, 0.023 mmol) in DMSO (2 mL) under nitrogen was stirred at 100° C. for 14 h. The mixture was diluted with ethyl acetate and washed by brine.

The organic phase was dried over anhydrous sodium sulfate, filtered and evaporated. The residue was purified by Prep-TLC (dichloromethane/methanol=15/1, v/v) then Prep-HPLC to give the title compound (7 mg, 6%). 1H NMR (400 MHz, d6-DMSO) δ 13.16 (s, 1H), 9.46 (s, 1H), 9.41 (s, 1H), 8.23 (d, J=8.6 Hz, 1H), 7.49 (dd, J=21.4, 8.3 Hz, 3H), 7.28-7.00 (m, 5H), 3.53 (s, 3H), 2.34 (s, 3H). LC-MS (M+H)⁺=504.3.

### Example 130: N-(5-(4-chloro-3-fluorophenoxy)thiazolo[5,4-b]pyridin-2-yl)-5-(2-methoxyphenyl)pyridazine-4-carboxamide

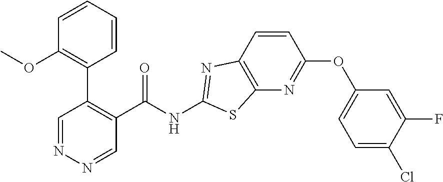 [SMILES: COc1ccccc1-c1cnncc1C(=O)Nc1nc2ccc(Oc3ccc(Cl)c(F)c3)nc2s1]

The title compound (3 mg, 2.6%) was prepared in a manner similar to Example 129 from N-(5-bromothiazolo[5,4-b]pyridin-2-yl)-5-(2-methoxyphenyl)pyridazine-4-carboxamide and 4-chloro-3-fluorophenol. 1H NMR (400 MHz, d6-DMSO) δ 13.19 (s, 1H), 9.47 (s, 1H), 9.42 (s, 1H), 8.27 (d, J=9.2 Hz, 1H), 7.65 (t, J=8.8 Hz, 1H), 7.59-7.38 (m, 3H), 7.28-7.04 (m, 4H), 3.53 (s, 3H). LC-MS (M+H)⁺=508.3.

### Example 131: N-(5-(4-chloro-3-methoxyphenoxy)thiazolo[5,4-b]pyridin-2-yl)-5-(2-methoxyphenyl)pyridazine-4-carboxamide

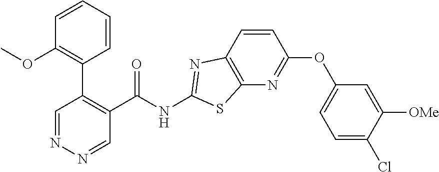 [SMILES: COc1cc(Oc2ccc3nc(NC(=O)c4cnncc4-c4ccccc4OC)sc3n2)ccc1Cl]

The title compound (5 mg, 4.2%) was prepared in a manner similar to Example 129 from N-(5-bromothiazolo[5,4-b]pyridin-2-yl)-5-(2-methoxyphenyl)pyridazine-4-carboxamide and 4-chloro-3-methoxyphenol. 1H NMR (400 MHz, d6-DMSO) δ 13.17 (s, 1H), 9.46 (s, 1H), 9.40 (s, 1H), 8.23 (s, 1H), 7.58-7.42 (m, 3H), 7.25-7.09 (m, 2H), 7.05 (t, J=7.5 Hz, 2H), 6.77 (d, J=6.4 Hz, 1H), 3.84 (s, 3H), 3.53 (s, 3H). LC-MS (M+H)⁺=520.3.

### Example 132: N-(5-(4-(ethylcarbamoyl)-2-methylphenyl)thiazolo[5,4-b]pyridin-2-yl)-5-(2-methoxyphenyl)pyridazine-4-carboxamide

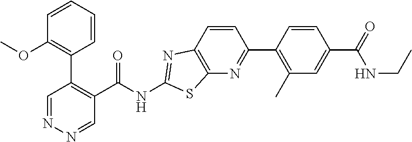 [SMILES: CCNC(=O)c1ccc(-c2ccc3nc(NC(=O)c4cnncc4-c4ccccc4OC)sc3n2)c(C)c1]

The title compound (22 mg, 19%) was prepared in a manner similar to that described in Example 78 of Step 3 from N-ethyl-3-methyl-4-(4,4,5,5-tetramethyl-1,3,2-dioxaborolan-2-yl)benzamide and N-(5-bromothiazolo[5,4-b]pyridin-2-yl)-5-(2-methoxyphenyl)pyridazine-4-carboxamide. 1H NMR (400 MHz, d6-DMSO)δ 13.30 (s, 1H), 9.50 (s, 1H), 9.41 (s, 1H), 8.51 (t, J=5.5 Hz, 1H), 8.20 (s, 1H), 7.81 (s, 1H), 7.77 (d, J=8.0 Hz, 1H), 7.68 (d, J=8.3 Hz, 1H), 7.54 (t, J=7.2 Hz, 2H), 7.49 (t, J=7.8 Hz, 1H), 7.15 (t, J=7.5 Hz, 1H), 7.08 (d, J=8.3 Hz, 1H), 3.57 (s, 3H), 3.35-3.25 (m, 2H), 2.42 (s, 3H), 1.15 (t, J=7.2 Hz, 3H). LC-MS (M+H)⁺=525.0.

### Example 133: N-(5-(2-fluoro-4-(methylsulfonyl)phenyl)thiazolo[5,4-b]pyridin-2-yl)-5-(2-methoxyphenyl)pyridazine-4-carboxamide

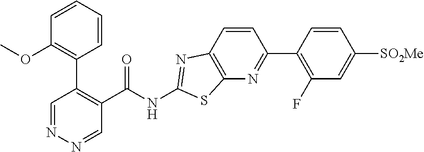 [SMILES: COc1ccccc1-c1cnncc1C(=O)Nc1nc2ccc(-c3ccc(S(C)(=O)=O)cc3F)nc2s1]

The title compound (15 mg, 12%) was prepared in a manner similar to that described in Example 78 of Step 3 from N-(5-bromothiazolo[5,4-b]pyridin-2-yl)-5-(2-methoxyphenyl)pyridazine-4-carboxamide and (2-fluoro-4-(methylsulfonyl)phenyl)boronic acid. 1H NMR (500 MHz, d6-DMSO) δ 13.41 (s, 1H), 9.51 (s, 1H), 9.42 (s, 1H), 8.27 (t, J=7.8 Hz, 2H), 8.00 (d, J=8.0 Hz, 1H), 7.96 (d, J=10.6 Hz, 1H), 7.91 (d, J=8.2 Hz, 1H), 7.54 (d, J=7.4 Hz, 1H), 7.49 (t, J=7.8 Hz, 1H), 7.15 (t, J=7.4 Hz, 1H), 7.07 (d, J=8.3 Hz, 1H), 3.56 (s, 3H), 3.34 (s, 3H) LC-MS (M+H)⁺=536.3.

### Example 134: 5-(2-methoxyphenyl)-N-(5-(2-methyl-4-(methylcarbamoyl)phenyl)thiazolo[5,4-b]pyridin-2-yl)pyridazine-4-carboxamide

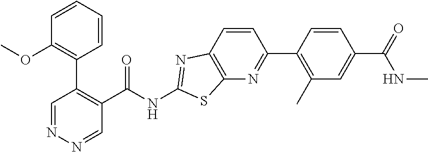

The title compound (23 mg, 16%) was prepared in a manner similar to that described in Example 78 step 3 from N-methyl-3-methyl-4-(4,4,5,5-tetramethyl-1,3,2-dioxaborolan-2-yl)benzamide and N-(5-bromothiazolo[5,4-b]pyridin-2-yl)-5-(2-methoxyphenyl)pyridazine-4-carboxamide. 1H NMR (400 MHz, d6-DMSO) δ13.30 (s, 1H), 9.50 (s, 1H), 9.42 (s, 1H), 8.49 (d, J=4.4 Hz, 1H), 8.22 (d, J=8.1 Hz, 1H), 7.81 (s, 1H), 7.76 (d, J=8.2 Hz, 1H), 7.69 (d, J=8.4 Hz, 1H), 7.62-7.52 (m, 2H), 7.49 (t, J=7.9 Hz, 1H), 7.15 (t, J=7.5 Hz, 1H), 7.08 (d, J=8.3 Hz, 1H), 3.57 (s, 3H), 2.81 (d, J=4.3 Hz, 3H), 2.41 (s, 3H). LC-MS (M+H)⁺=511.0.

### Example 135: 4-(5-cyano-2-methoxyphenyl)-N-(5-(4-(1,1-dioxidoisothiazolidin-2-yl)phenyl)thiazolo[5,4-b]pyridin-2-yl)-6-methylnicotinamide

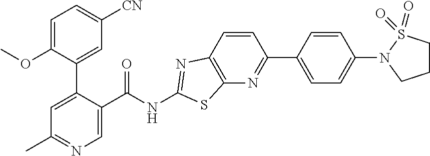 [SMILES: COc1ccc(C#N)cc1-c1cc(C)ncc1C(=O)Nc1nc2ccc(-c3ccc(N4CCCS4(=O)=O)cc3)nc2s1]

The title compound (26 mg, 27%) was prepared in a manner similar to that in Example 78 step 3 from 2-(4-(4,4,5,5-tetramethyl-1,3,2-dioxaborolan-2-yl)phenyl)isothiazolidine 1,1-dioxide and N-(5-bromothiazolo[5,4-b]pyridin-2-yl)-4-(5-cyano-2-methoxyphenyl)-6-methylnicotinamide. ¹H NMR (300 MHz, d6-DMSO) δ 12.95 (brs, 1H), 8.80 (s, 1H), 8.21-8.09 (m, 3H), 8.05 (d, J=8.6 Hz, 1H), 7.96-7.86 (m, 2H), 7.44 (s, 1H), 7.37-7.26 (m, 2H), 7.23-7.13 (m, 1H), 3.82 (t, J=6.5 Hz, 2H), 3.63-3.51 (m, 4H), 2.60 (s, 3H), 2.43 (p, J=7.0 Hz, 2H). LC-MS (M+H)⁺=597.0.

### Example 136: 4-(5-cyano-2-methoxyphenyl)-N-(5-(4-(N-(2-hydroxyethyl)methylsulfonamido)phenyl)thiazolo[5,4-b]pyridin-2-yl)-6-methylnicotinamide

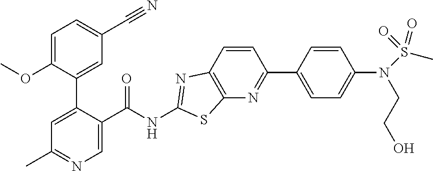

### Step 1: N-(4-bromophenyl)-N-(2-((tert-butyldimethylsilyl)oxy)ethyl)methanesulfonamide

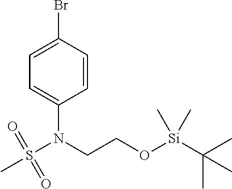 [SMILES: CC(C)(C)[Si](C)(C)OCCN(c1ccc(Br)cc1)S(C)(=O)=O]

The title compound (900 mg, 54%) was prepared in a manner similar to that in Example 87 step 1 from N-(4-bromophenyl)methanesulfonamide and (2-bromoethoxy)(tert-butyl)dimethylsilane. LC-MS (M+H)⁺=408.0.

### Step 2: N-(2-((tert-butyldimethylsilyl)oxy)ethyl)-N-(4-(4,4,5,5-tetramethyl-1,3,2-dioxaborolan-2-yl)phenyl)methanesulfonamide

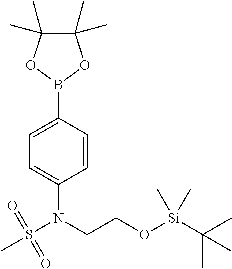

The title compound (328 mg, 59%) was prepared in a manner similar to that in Example 28 step 2 from N-(4-bromophenyl)-N-(2-((tert-butyldimethylsilyl)oxy)ethyl)methanesulfonamide. LC-MS (M+H)⁺=456.1.

### Step 3: tert-butyl (5-(4-(N-(2-((tert-butyldimethylsilyl)oxy)ethyl)methylsulfonamido)phenyl)thiazolo[5,4-b]pyridin-2-yl)carbamate

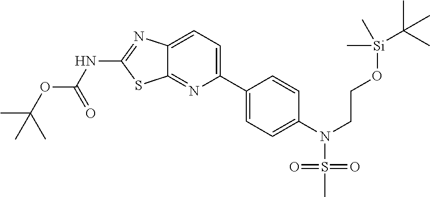 [SMILES: CC(C)(C)OC(=O)Nc1nc2ccc(-c3ccc(N(CCO[Si](C)(C)C(C)(C)C)S(C)(=O)=O)cc3)nc2s1]

The title compound (96 mg, 50%) was prepared in a manner similar to that in Example 78 step 3 from N-(2-((tert-butyldimethylsilyl)oxy)ethyl)-N-(4-(4,4,5,5-tetramethyl-1,3,2-dioxaborolan-2-yl)phenyl)methanesulfonamide and tert-butyl (5-bromothiazolo[5,4-b]pyridin-2-yl)carbamate. LC-MS (M+H)⁺=579.5.

### Step 4: N-(4-(2-aminothiazolo[5,4-b]pyridin-5-yl)phenyl)-N-(2-hydroxyethyl)methanesulfonamide

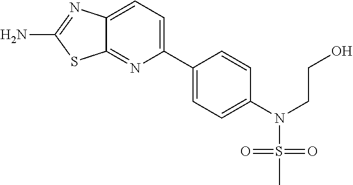 [SMILES: CS(=O)(=O)N(CCO)c1ccc(-c2ccc3nc(N)sc3n2)cc1]

The title compound (60 mg, 90%) was prepared in a manner similar to that in Example 1 step 6 from tert-butyl (5-(4-(N-(2-((tert-butyldimethylsilyl)oxy)ethyl)methylsulfonamido)phenyl)thiazolo[5,4-b]pyridin-2-yl)carbamate. LC-MS (M+H)⁺=365.1.

### Step 5: 4-(5-cyano-2-methoxyphenyl)-N-(5-(4-(N-(2-hydroxyethyl)methylsulfonamido)phenyl)thiazolo[5,4-b]pyridin-2-yl)-6-methylnicotinamide

The title compound (16 mg, 19%) was prepared in a manner similar to that in Example 81 step 2 from N-(4-(2-aminothiazolo[5,4-b]pyridin-5-yl)phenyl)-N-(2-hydroxyethyl)methanesulfonamide and 4-(5-cyano-2-methoxyphenyl)-6-methylnicotinic acid. ¹H NMR (400 MHz, d6-DMSO) δ 13.02 (brs, 1H), 8.80 (s, 1H), 8.26-8.04 (m, 4H), 7.96-7.89 (m, 2H), 7.57-7.50 (m, 2H), 7.45 (s, 1H), 7.22-7.15 (m, 1H), 4.84 (brs, 1H), 3.73 (t, J=6.2 Hz, 2H), 3.60 (s, 3H), 3.43 (t, J=6.2 Hz, 2H), 3.07 (s, 3H), 2.60 (s, 3H). LC-MS (M+H)⁺=615.2.

### Example 137: N-(5-(4-chlorobenzoyl)thiazolo[5,4-b]pyridin-2-yl)-5-(2-methoxyphenyl)pyridazine-4-carboxamide

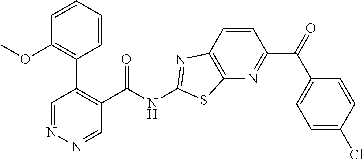 [SMILES: COc1ccccc1-c1cnncc1C(=O)Nc1nc2ccc(C(=O)c3ccc(Cl)cc3)nc2s1]

### Step 1: tert-butyl (5-bromothiazolo[5,4-b]pyridin-2-yl)((2-(trimethylsilyl)ethoxy)methyl)carbamate

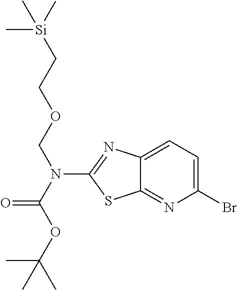 [SMILES: CC(C)(C)OC(=O)N(COCC[Si](C)(C)C)c1nc2ccc(Br)nc2s1]

To a solution of tert-butyl (5-bromothiazolo[5,4-b]pyridin-2-yl)carbamate (1 g, 3.03 mmol) in N,N-dimethylformamide (11.5 mL) was added t-BuOK (510 mg, 4.55 mmol) at 0° C. The mixture was stirred at 0° C. for 0.5 h under nitrogen and then 2-(trimethylsilyl)ethoxymethyl chloride (610 mg, 3.64 mmol) was added dropwise. The resulting mixture was stirred at 25° C. for 2 h under nitrogen. Saturated NH₄Cl was used to quench the reaction and extracted with ethyl acetate (20 mL), washed with brine (20 mL), dried with over sodium sulfate, and concentrated under reduced pressure. The residue was purified by silica gel column chromatography, eluting with ethyl acetate in petroleum ether (33%, v/v) to give title compound (1.1 g, 79%). LC-MS (M+H)⁺=460.2.

### Step 2: tert-butyl (5-(4-chlorobenzoyl)thiazolo[5,4-b]pyridin-2-yl)((2-(trimethylsilyl)ethoxy)methyl)carbamate

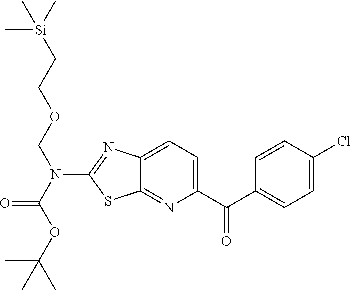 [SMILES: CC(C)(C)OC(=O)N(COCC[Si](C)(C)C)c1nc2ccc(C(=O)c3ccc(Cl)cc3)nc2s1]

To a mixture of tert-butyl (5-bromothiazolo[5,4-b]pyridin-2-yl)((2-(trimethylsilyl)ethoxy)methyl)carbamate (2.37 g, 5.152 mmol) and 4-chloro-N-methoxy-N-methylbenzamide (2.05 g, 10.3 mmol) in THF (33 mL) was added dropwise n-BuLi (1.6 N, 6.4 mL, 10.3 mmol) at −78° C. under nitrogen. The mixture was stirred at −78° C. for 2 h and then at 25° C. for 18 h. Saturated NH₄Cl was used to quench the reaction and extracted with ethyl acetate (20 mL), washed with brine (20 mL), dried with over sodium sulfate, and concentrated under reduced pressure. The residue was purified by silica gel column chromatography, eluting with ethyl acetate in petroleum ether (33%, v/v) to give title compound (0.75 g, 26%). LC-MS (M+H)⁺=520.2.

### Step 3: (2-aminothiazolo[5,4-b]pyridin-5-yl)(4-chlorophenyl)methanone

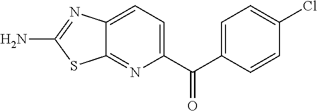 [SMILES: Nc1nc2ccc(C(=O)c3ccc(Cl)cc3)nc2s1]

The title compound (97 mg, 90%) was prepared in a manner similar to that in Example 1 step 6 from tert-butyl (5-(4-chlorobenzoyl)thiazolo[5,4-b]pyridin-2-yl)((2-(trimethylsilyl)ethoxy)methyl)carbamate. LC-MS (M+H)⁺=290.0.

### Step 4: N-(5-(4-chlorobenzoyl)thiazolo[5,4-b]pyridin-2-yl)-5-(2-methoxyphenyl)pyridazine-4-carboxamide

The title compound (11 mg, 12%) was prepared in a manner similar to that in Example 11 step 3 from 5-(2-methoxyphenyl)pyridazine-4-carboxylic acid and (2-aminothiazolo[5,4-b]pyridin-5-yl)(4-chlorophenyl)methanone. ¹H NMR (400 MHz, d6-DMSO) δ:13.54 (s, 1H), 9.51 (s, 1H), 9.44 (s, 1H), 8.36 (d, J=8.5 Hz, 1H), 8.19 (d, J=8.4 Hz, 1H), 8.07 (d, J=8.4 Hz, 2H), 7.64 (d, J=8.4 Hz, 2H), 7.54 (d, J=7.5 Hz, 1H), 7.49 (t, J=8.0 Hz, 1H), 7.15 (t, J=7.5 Hz, 1H), 7.07 (d, J=8.4 Hz, 1H), 3.55 (s, 3H). LC-MS (M+H)⁺=502.4.

### Example 138: N-(5-(1-(4-chlorophenyl)ethyl)thiazolo[5,4-b]pyridin-2-yl)-5-(2-methoxyphenyl)pyridazine-4-carboxamide

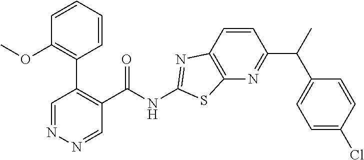 [SMILES: COc1ccccc1-c1cnncc1C(=O)Nc1nc2ccc(C(C)c3ccc(Cl)cc3)nc2s1]

### Step 1: tert-butyl (5-(1-(4-chlorophenyl)vinyl)thiazolo[5,4-b]pyridin-2-yl)((2-(trimethylsilyl)ethoxy)methyl)carbamate

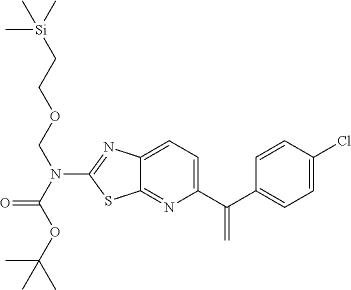 [SMILES: C=C(c1ccc(Cl)cc1)c1ccc2nc(N(COCC[Si](C)(C)C)C(=O)OC(C)(C)C)sc2n1]

To a solution of methyltriphenylphosphonium bromide (714 mg, 2 mmol) in THF (4 mL) was added t-BuOK (269 mg, 2.4 mmol) at 0° C. The mixture was stirred at 0° C. for 0.5 h under nitrogen and then a solution of tert-butyl (5-(4-chlorobenzoyl)thiazolo[5,4-b]pyridin-2-yl)((2-(trimethylsilyl)ethoxy)methyl)carbamate (519 mg, 1 mmol) was added dropwise. The resulting mixture was stirred at 50° C. for 18 h under nitrogen. Saturated NH₄Cl was used to quench the reaction and extracted with ethyl acetate (20 mL), washed with brine (20 mL), dried with over sodium sulfate, and concentrated under reduced pressure. The residue was purified by silica gel column chromatography, eluting with ethyl acetate in petroleum ether (33%, v/v) to give compound 14-1 (469 mg, 90%). LC-MS (M+H)⁺=518.3.

### Step 2: tert-butyl (5-(1-(4-chlorophenyl)ethyl)thiazolo[5,4-b]pyridin-2-yl)((2-(trimethylsilyl)ethoxy)methyl)carbamate

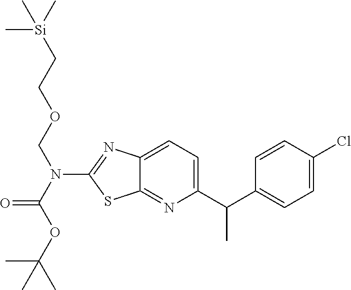 [SMILES: CC(c1ccc(Cl)cc1)c1ccc2nc(N(COCC[Si](C)(C)C)C(=O)OC(C)(C)C)sc2n1]

The title compound (0.2 g, 97%) was prepared in a manner similar to that in Example 67 step 2 from tert-butyl (5-(1-(4-chlorophenyl)vinyl)thiazolo[5,4-b]pyridin-2-yl)((2-(trimethylsilyl)ethoxy)methyl)carbamate. LC-MS (M+H)⁺=520.3.

### Step 3: 5-(1-(4-chlorophenyl)ethyl)thiazolo[5,4-b]pyridin-2-amine

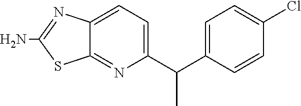 [SMILES: CC(c1ccc(Cl)cc1)c1ccc2nc(N)sc2n1]

To a mixture of tert-butyl (5-(1-(4-chlorophenyl)ethyl)thiazolo[5,4-b]pyridin-2-yl)((2-(trimethylsilyl)ethoxy)methyl)carbamate (200 mg, 0.384 mmol) in dichloromethane (9 mL) was added CF₃COOH (6 mL). The mixture was stirred at room temperature for 18 h. The mixture was concentrated under reduced pressure and washed with saturated NaHCO₃ to give compound 14-3 (100 mg, crude). LC-MS (M+H)⁺=290.1.

### Step 4: N-(5-(1-(4-chlorophenyl)ethyl)thiazolo[5,4-b]pyridin-2-yl)-5-(2-methoxyphenyl)pyridazine-4-carboxamide

To a solution of 5-(2-methoxyphenyl)pyridazine-4-carboxylic acid (96 mg, 0.415 mmol) and 5-(1-(4-chlorophenyl)ethyl)thiazolo[5,4-b]pyridin-2-amine (100 mg, 0.346 mmol) in pyridine (10 mL) was added EDCI (398 mg, 2.076 mmol) and HOBt (281 mg, 2.076 mmol) under nitrogen. The mixture was stirred at 80° C. for 18 hours under nitrogen. Water (10 mL) was added to quench the reaction, extracted with ethyl acetate (10 mL), washed with brine (10 mL), dried with over sodium sulfate, and concentrated under reduced pressure. The residue was purified by silica gel column chromatography eluting with methanol in dichloromethane (5%, v/v) to afford the title compound (30 mg, 14%). ¹H NMR (400 MHz, d6-DMSO) δ:13.16 (s, 1H), 9.47 (s, 1H), 9.41 (s, 1H), 8.06 (d, J=8.3 Hz, 1H), 7.57-7.40 (m, 3H), 7.40-7.30 (m, 4H), 7.14 (t, J=7.5 Hz, 1H), 7.05 (d, J=8.4 Hz, 1H), 4.44 (q, J=7.0 Hz, 1H), 3.53 (s, 3H), 1.65 (d, J=7.1 Hz, 3H). LC-MS (M+H)⁺=502.4.

### Example 139: N-(5-(4-chloro-2,3-dimethylphenyl)thiazolo[5,4-b]pyridin-2-yl)-5-(2-methoxyphenyl)pyridazine-4-carboxamide

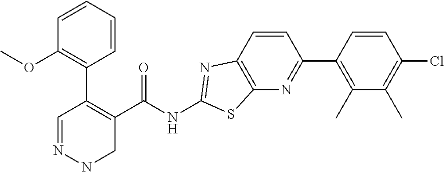 [SMILES: COc1ccccc1C1=C(C(=O)Nc2nc3ccc(-c4ccc(Cl)c(C)c4C)nc3s2)CNN=C1]

The title compound (48 mg, 48%) was prepared in a manner similar to that in Example 78 step 3 from N-(5-bromothiazolo[5,4-b]pyridin-2-yl)-5-(2-methoxyphenyl)pyridazine-4-carboxamide and 2-(4-chloro-2,3-dimethylphenyl)-4,4,5,5-tetramethyl-1,3,2-dioxaborolane. ¹H NMR (400 MHz, d6-DMSO) δ: 13.28 (s, 1H), 9.50 (s, 1H), 9.43 (s, 1H), 8.22 (d, J=8.1 Hz, 1H), 7.62-7.45 (m, 3H), 7.38 (d, J=8.3 Hz, 1H), 7.26 (d, J=8.3 Hz, 1H), 7.15 (t, J=7.4 Hz, 1H), 7.07 (d, J=8.3 Hz, 1H), 3.57 (s, 3H), 2.39 (s, 3H), 2.25 (s, 3H). LC-MS (M+H)⁺=502.3.

### Example 140: N-(5-(4-acetamido-3,5-dimethylphenyl)thiazolo[5,4-b]pyridin-2-yl)-5-(2-methoxyphenyl)pyridazine-4-carboxamide

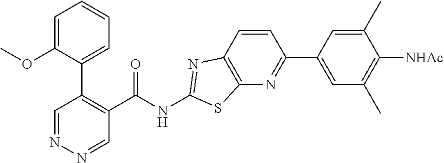 [SMILES: COc1ccccc1-c1cnncc1C(=O)Nc1nc2ccc(-c3cc(C)c(NC(C)=O)c(C)c3)nc2s1]

### Step 1: N-(4-bromo-2,6-dimethylphenyl)acetamide

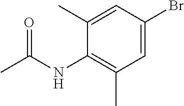

To a solution of 4-bromo-2,6-dimethylaniline (2 g, 10 mmol) and triethylamine (2.02 g, 20 mmol) in dichloroethane (20 mL) was added acetyl chloride (0.94 g, 12 mmol) and the reaction solution was stirred at room temperature for 14 h before diluted with dichloromethane. The solution was washed with water and brine, then dried over anhydrous sodium sulfate, filtered and evaporated. Purification by silica gel column chromatography gave the title compound (1.8 g, 75%). LC-MS (M+H)⁺=242.1, 244.1.

### Step 2: N-(2,6-dimethyl-4-(4,4,5,5-tetramethyl-1,3,2-dioxaborolan-2-yl)phenyl)acetamide

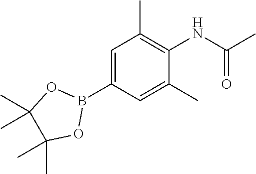 [SMILES: CC(=O)Nc1c(C)cc(B2OC(C)(C)C(C)(C)O2)cc1C]

To a solution of N-(4-bromo-2,6-dimethylphenyl)acetamide (400 mg, 1.65 mmol) and 4,4,4′,4′,5,5,5′,5′-octamethyl-2,2′-bi(1,3,2-dioxaborolane (504 mg, 1.98 mmol) in dioxane (20 mL) were added [1,1′-Bis(diphenylphosphino)ferrocene]dichloropalladium(II) (120 mg, 0.165 mmol) and potassium acetate (323 mg, 3.3 mmol). The resulting mixture was heated to reflux for 15 h under nitrogen atmosphere before cooled to room temperature and diluted with ethyl acetate (50 mL). The mixture was washed with brine (50 mL), dried over sodium sulfate, filtered and concentrated under reduced pressure. The residue was purified by silica gel column chromatography, eluting with ethyl acetate in petroleum ether (17%, v/v) to give title compound (380 mg, 80%). LC-MS (M+H)⁺=290.0.

### Step 3: N-(5-(4-acetamido-3,5-dimethylphenyl)thiazolo[5,4-b]pyridin-2-yl)-5-(2-methoxyphenyl)pyridazine-4-carboxamide

The title compound (10 mg, 8.5%) was prepared in a manner similar to Example 96 step 2 from N-(5-bromothiazolo[5,4-b]pyridin-2-yl)-5-(2-methoxyphenyl)pyridazine-4-carboxamide and N-(2,6-dimethyl-4-(4,4,5,5-tetramethyl-1,3,2-dioxaborolan-2-yl)phenyl)acetamide. ¹H NMR (400 MHz, d6-DMSO) δ 13.25 (s, 1H), 9.49 (s, 1H), 9.42 (s, 1H), 9.32 (s, 1H), 8.19 (d, J=8.3 Hz, 1H), 8.06 (d, J=8.5 Hz, 1H), 7.83 (s, 2H), 7.54 (d, J=7.2 Hz, 1H), 7.49 (t, J=7.7 Hz, 1H), 7.15 (t, J=7.3 Hz, 1H), 7.07 (d, J=8.1 Hz, 1H), 3.56 (s, 3H), 2.24 (s, 6H), 2.07 (s, 3H). LC-MS (M+H)⁺=525.1.

### Example 141: N-(5-(4-acetamido-2,5-dimethylphenyl)thiazolo[5,4-b]pyridin-2-yl)-5-(2-methoxyphenyl)pyridazine-4-carboxamide

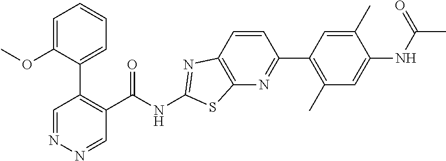 [SMILES: COc1ccccc1-c1cnncc1C(=O)Nc1nc2ccc(-c3cc(C)c(NC(C)=O)cc3C)nc2s1]

### Step 1: N-(4-bromo-2,5-dimethylphenyl)acetamide

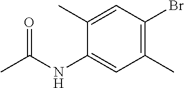

The title compound (1.8 g, 75%) was prepared in a manner similar to Example 140 step 1 from 4-bromo-2,5-dimethylaniline. LC-MS (M+H)⁺=242.1, 244.1.

### Step 2: N-(2,5-dimethyl-4-(4,4,5,5-tetramethyl-1,3,2-dioxaborolan-2-yl)phenyl)acetamide

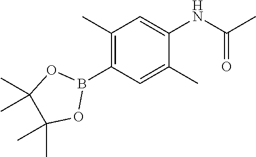 [SMILES: CC(=O)Nc1cc(C)c(B2OC(C)(C)C(C)(C)O2)cc1C]

The title compound (0.9 g, 38%) was prepared in a manner similar to Example 28 step 2 from N-(4-bromo-2,5-dimethylphenyl)acetamide. LC-MS (M+H)⁺=290.1.

### Step 3: N-(5-(4-acetamido-2,5-dimethylphenyl)thiazolo[5,4-b]pyridin-2-yl)-5-(2-methoxyphenyl)pyridazine-4-carboxamide

The title compound (9 mg, 7%) was prepared in a manner similar to Example 96 step 2 from N-(5-bromothiazolo[5,4-b]pyridin-2-yl)-5-(2-methoxyphenyl)pyridazine-4-carboxamide and N-(2,5-dimethyl-4-(4,4,5,5-tetramethyl-1,3,2-dioxaborolan-2-yl)phenyl)acetamide. ¹H NMR (400 MHz, d6-DMSO) δ 13.25 (s, 1H), 9.49 (s, 1H), 9.41 (s, 1H), 9.31 (s, 1H), 8.17 (s, 1H), 7.62 (d, J=8.5 Hz, 1H), 7.53 (d, J=6.9 Hz, 1H), 7.49 (t, J=8.1 Hz, 1H), 7.40 (s, 1H), 7.31 (s, 1H), 7.15 (t, J=7.4 Hz, 1H), 7.07 (d, J=8.1 Hz, 1H), 3.57 (s, 3H), 2.31 (s, 3H), 2.22 (s, 3H), 2.08 (s, 3H). LC-MS (M+H)⁺=525.1.

### Example 142: N-(5-(4-acetamido-3-fluorophenyl)thiazolo[5,4-b]pyridin-2-yl)-5-(2-methoxyphenyl)pyridazine-4-carboxamide

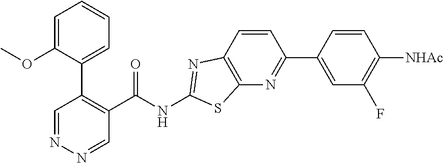 [SMILES: COc1ccccc1-c1cnncc1C(=O)Nc1nc2ccc(-c3ccc(NC(C)=O)c(F)c3)nc2s1]

### Step 1: N-(4-bromo-2-fluorophenyl)acetamide

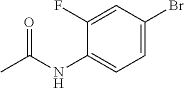

The title compound (2 g, 86%) was prepared in a manner similar to Example 140 step 1 from 4-bromo-2-fluoroaniline. LC-MS (M+H)⁺=231.9, 233.9.

### Step 2: N-(2-fluoro-4-(4,4,5,5-tetramethyl-1,3,2-dioxaborolan-2-yl)phenyl)acetamide

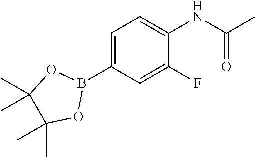 [SMILES: CC(=O)Nc1ccc(B2OC(C)(C)C(C)(C)O2)cc1F]

The title compound (0.5 g, 42%) was prepared in a manner similar to Example 28 step 2 from N-(4-bromo-2-fluorophenyl)acetamide. LC-MS (M+H)⁺=280.1.

### Step 3: N-(5-(4-acetamido-3-fluorophenyl)thiazolo[5,4-b]pyridin-2-yl)-5-(2-methoxyphenyl)pyridazine-4-carboxamide

The title compound (9 mg, 7.8%) was prepared in a manner similar to Example 96 step 2 from N-(5-bromothiazolo[5,4-b]pyridin-2-yl)-5-(2-methoxyphenyl)pyridazine-4-carboxamide and N-(2-fluoro-4-(4,4,5,5-tetramethyl-1,3,2-dioxaborolan-2-yl)phenyl)acetamide. ¹H NMR (400 MHz, d6-DMSO) δ 13.26 (s, 1H), 9.86 (s, 1H), 9.49 (s, 1H), 9.39 (s, 1H), 8.13 (dt, J=17.7, 8.3 Hz, 3H), 8.00 (d, J=12.5 Hz, 1H), 7.95 (d, J=8.8 Hz, 1H), 7.50 (dd, J=19.3, 7.5 Hz, 2H), 7.14 (t, J=7.5 Hz, 1H), 7.07 (d, J=8.2 Hz, 1H), 3.56 (s, 3H), 2.13 (s, 3H). LC-MS (M+H)⁺=515.1.

### Example 143: N-(5-(4-chloro-2-isopropoxyphenyl)thiazolo[5,4-b]pyridin-2-yl)-5-(2-methoxyphenyl)pyridazine-4-carboxamide

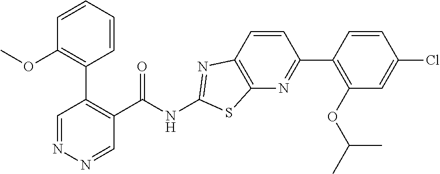 [SMILES: COc1ccccc1-c1cnncc1C(=O)Nc1nc2ccc(-c3ccc(Cl)cc3OC(C)C)nc2s1]

The title compound (117 mg, 49%) was prepared in a manner similar to that described in Example 78 step 3 from N-(5-bromothiazolo[5,4-b]pyridin-2-yl)-5-(2-methoxyphenyl)pyridazine-4-carboxamide and (4-chloro-2-isopropoxyphenyl)boronic acid. 1H NMR (400 MHz, d6-DMSO) δ 13.26 (s, 1H), 9.49 (s, 1H), 9.42 (s, 1H), 8.16 (d, J=8.4 Hz, 1H), 8.02 (d, J=8.7 Hz, 1H), 7.83 (d, J=8.3 Hz, 1H), 7.54 (d, J=7.4 Hz, 1H), 7.49 (t, J=7.9 Hz, 1H), 7.26 (s, 1H), 7.14 (dd, J=15.9, 8.4 Hz, 2H), 7.07 (d, J=8.5 Hz, 1H), 4.99-4.61 (m, 1H), 3.56 (s, 3H), 1.29 (d, J=5.9 Hz, 6H). LC-MS (M+H)⁺=533.0.

### Example 144: N-(5-(6-cyanopyridin-3-yl)thiazolo[5,4-b]pyridin-2-yl)-5-(2-methoxyphenyl)pyridazine-4-carboxamide

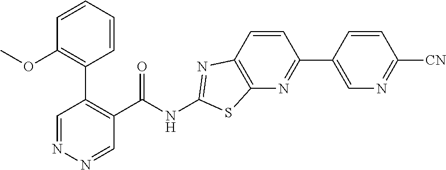 [SMILES: COc1ccccc1-c1cnncc1C(=O)Nc1nc2ccc(-c3ccc(C#N)nc3)nc2s1]

The title compound (12 mg, 11%) was prepared in a manner similar to that in Example 78 step 3 from N-(5-bromothiazolo[5,4-b]pyridin-2-yl)-5-(2-methoxyphenyl)pyridazine-4-carboxamide and (6-cyanopyridin-3-yl)boronic acid. 1H NMR (400 MHz, d6-DMSO) δ 13.40 (s, 1H), 9.50 (s, 2H), 9.42 (s, 1H), 8.75 (d, J=8.1 Hz, 1H), 8.31 (s, 2H), 8.18 (d, J=8.1 Hz, 1H), 7.54 (d, J=6.9 Hz, 1H), 7.51 (t, 1H), 7.15 (t, J=7.5 Hz, 1H), 7.07 (d, J=8.4 Hz, 1H), 3.56 (s, 3H). LC-MS (M+H)⁺=466.3.

### Example 145: N-(5-(4-(azetidine-1-carbonyl)phenyl)thiazolo[5,4-b]pyridin-2-yl)-4-(5-cyano-2-methoxyphenyl)-6-methylnicotinamide

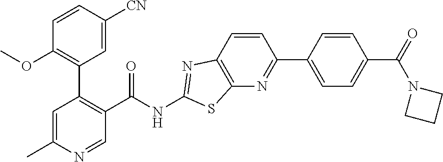 [SMILES: COc1ccc(C#N)cc1-c1cc(C)ncc1C(=O)Nc1nc2ccc(-c3ccc(C(=O)N4CCC4)cc3)nc2s1]

The title compound (13 mg, 12%) was prepared in a manner similar to that in Example 73 step 5 from 4-(2-(4-(5-cyano-2-methoxyphenyl)-6-methylnicotinamido)thiazolo[5,4-b]pyridin-5-yl)benzoic acid and azetidine. 1H NMR (400 MHz, d6-DMSO) δ 12.99 (s, 1H), 8.82 (s, 1H), 8.20 (d, J=8.0 Hz, 3H), 8.14 (d, J=4.9 Hz, 1H), 7.91 (d, J=9.7 Hz, 2H), 7.75 (d, J=8.0 Hz, 2H), 7.42 (s, 1H), 7.18 (d, J=8.5 Hz, 1H), 4.34 (d, J=6.8 Hz, 2H), 4.07 (t, J=7.5 Hz, 2H), 3.60 (s, 3H), 2.60 (s, 3H), 2.39-2.20 (m, 2H). LC-MS (M+H)⁺=561.6.

### Example 146: 4-(2-chloro-6-methoxyphenyl)-N-(5-(4-cyanophenyl)thiazolo[5,4-b]pyridin-2-yl)-6-methylnicotinamide

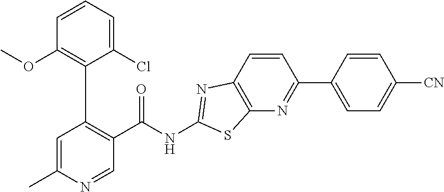 [SMILES: COc1cccc(Cl)c1-c1cc(C)ncc1C(=O)Nc1nc2ccc(-c3ccc(C#N)cc3)nc2s1]

### Step 1: methyl 4-(2-chloro-6-methoxyphenyl)-6-methylnicotinate

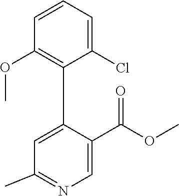

The title compound (245 mg, 68%) was prepared in a manner similar to that described in Example 2 step 2 from methyl 4-chloro-6-methylnicotinate and 2-(2-chloro-6-methoxyphenyl)-4,4,5,5-tetramethyl-1,3,2-dioxaborolane. LC-MS (M+H)⁺=292.1

### Step 2: 4-(2-chloro-6-methoxyphenyl)-6-methylnicotinic acid

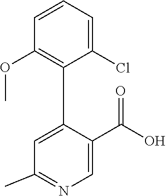 [SMILES: COc1cccc(Cl)c1-c1cc(C)ncc1C(=O)O]

The title compound (155 mg, 85%) was prepared in a manner similar to that described in Example 1 step 3 from methyl 4-(2-chloro-6-methoxyphenyl)-6-methylnicotinate. LC-MS (M+H)⁺=278.0.

### Step 3: 4-(2-chloro-6-methoxyphenyl)-N-(5-(4-cyanophenyl)thiazolo[5,4-b]pyridin-2-yl)-6-methylnicotinamide

The title compound (9 mg, 5.5%) was prepared in a manner similar to that described in Example 11 step 3 from 4-(2-chloro-6-methoxyphenyl)-6-methylnicotinic acid and 4-(2-aminothiazolo[5,4-b]pyridin-5-yl)benzonitrile. 1H NMR (400 MHz, d6-DMSO) δ: 13.14 (s, 1H), 8.96 (s, 1H), 8.35-8.28 (m, 2H), 8.26-8.21 (m, 2H), 8.02-7.89 (m, 2H), 7.39 (t, J=8.0 Hz, 1H), 7.27 (s, 1H), 7.15 (d, J=8.0 Hz, 1H), 7.05 (d, J=8.0 Hz, 1H), 3.61 (s, 3H), 2.59 (s, 3H). LC-MS (M+H)⁺=512.3.

### Example 147: 5-cyano-N-(5-(4-cyanophenyl)thiazolo[5,4-b]pyridin-2-yl)-1,6′-dimethyl-6-oxo-1,6-dihydro-[3,4′-bipyridine]-3′-carboxamide

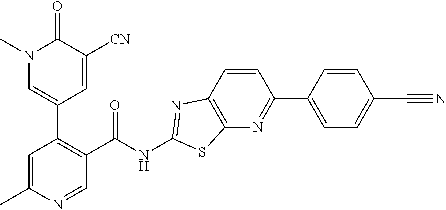 [SMILES: Cc1cc(-c2cc(C#N)c(=O)n(C)c2)c(C(=O)Nc2nc3ccc(-c4ccc(C#N)cc4)nc3s2)cn1]

### Step 1: 5-bromo-1-methyl-2-oxo-1,2-dihydropyridine-3-carbonitrile

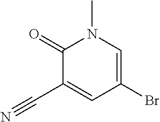

The title compound (651 mg, 61%) was prepared in a manner similar to that described in Example 87 step 1 from 5-bromo-2-oxo-1,2-dihydropyridine-3-carbonitrile and iodomethane. LC-MS (M+H)⁺=213.0.

### Step 2: 1-methyl-2-oxo-5-(4,4,5,5-tetramethyl-1,3,2-dioxaborolan-2-yl)-1,2-dihydropyridine-3-carbonitrile

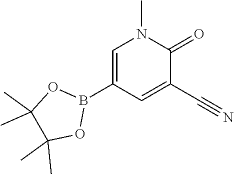 [SMILES: Cn1cc(B2OC(C)(C)C(C)(C)O2)cc(C#N)c1=O]

The title compound (491 mg, 65%) was prepared in a manner similar to that described in Example 28 step 2 from 5-bromo-1-methyl-2-oxo-1,2-dihydropyridine-3-carbonitrile. LC-MS (M+H)⁺=261.1.

### Step 3: methyl 5-cyano-1,6′-dimethyl-6-oxo-1,6-dihydro-[3,4′-bipyridine]-3′-carboxylate

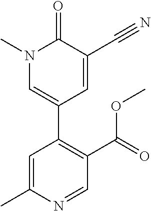 [SMILES: COC(=O)c1cnc(C)cc1-c1cc(C#N)c(=O)n(C)c1]

The title compound (283 mg, 82%) was prepared in a manner similar to that described in Example 2 step 2 from methyl 4-chloro-6-methylnicotinate and 1-methyl-2-oxo-5-(4,4,5,5-tetramethyl-1,3,2-dioxaborolan-2-yl)-1,2-dihydropyridine-3-carbonitrile. LC-MS (M+H)⁺=284.1

### Step 4: 5-cyano-1,6′-dimethyl-6-oxo-1,6-dihydro-[3,4′-bipyridine]-3′-carboxylic acid

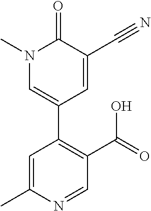 [SMILES: Cc1cc(-c2cc(C#N)c(=O)n(C)c2)c(C(=O)O)cn1]

The title compound (140 mg, 85%) was prepared in a manner similar to that described in Example 1 step 3 from methyl 5-cyano-1,6′-dimethyl-6-oxo-1,6-dihydro-[3,4′-bipyridine]-3′-carboxylate. LC-MS (M+H)⁺=270.0.

### Step 5: 5-cyano-N-(5-(4-cyanophenyl)thiazolo[5,4-b]pyridin-2-yl)-1,6′-dimethyl-6-oxo-1,6-dihydro-[3,4′-bipyridine]-3′-carboxamide

The title compound (13.5 mg, 6.5%) was prepared in a manner similar to that described in Example 11 step 3 from 5-cyano-1,6′-dimethyl-6-oxo-1,6-dihydro-[3,4′-bipyridine]-3′-carboxylic acid and 4-(2-aminothiazolo[5,4-b]pyridin-5-yl)benzonitrile. ¹H NMR (400 MHz, d6-DMSO) δ: 13.18 (s, 1H), 8.90 (s, 1H), 8.40-8.30 (m, 3H), 8.28-8.18 (m, 3H), 8.03-7.95 (m, 2H), 7.47 (s, 1H), 3.56 (s, 3H), 2.59 (s, 3H). LC-MS (M+H)⁺=504.1.

### Example 148: N-(5-(4-cyano-2,6-dimethylphenyl)thiazolo[5,4-b]pyridin-2-yl)-5-(2-methoxyphenyl)pyridazine-4-carboxamide

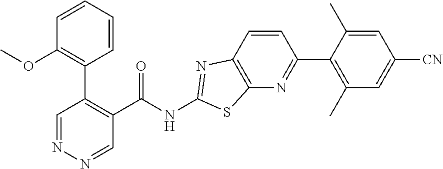 [SMILES: COc1ccccc1-c1cnncc1C(=O)Nc1nc2ccc(-c3c(C)cc(C#N)cc3C)nc2s1]

The title compound (49 mg, 49%) was prepared in a manner similar to that in Example 78 step 3 from N-(5-bromothiazolo[5,4-b]pyridin-2-yl)-5-(2-methoxyphenyl)pyridazine-4-carboxamide and 3,5-dimethyl-4-(4,4,5,5-tetramethyl-1,3,2-dioxaborolan-2-yl)benzonitrile. ¹H NMR (400 MHz, d6-DMSO) δ: 13.34 (s, 1H), 9.49 (s, 1H), 9.44 (s, 1H), 8.28 (d, J=8.3 Hz, 1H), 7.67 (s, 2H), 7.58-7.44 (m, 3H), 7.16 (t, J=7.5 Hz, 1H), 7.09 (d, J=8.4 Hz, 1H), 3.58 (s, 6H), 2.04 (s, 3H). LC-MS (M+H)⁺=493.2.

### Example 149: 5-(2-methoxyphenyl)-N-(5-(4-(4-oxooxazolidin-3-yl)phenyl)thiazolo[5,4-b]pyridin-2-yl)pyridazine-4-carboxamide

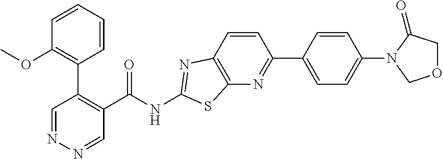 [SMILES: COc1ccccc1-c1cnncc1C(=O)Nc1nc2ccc(-c3ccc(N4COCC4=O)cc3)nc2s1]

### Step 1: N-(4-bromophenyl)-2-hydroxyacetamide

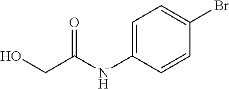 [SMILES: O=C(CO)Nc1ccc(Br)cc1]

A solution of 2-((4-bromophenyl)amino)-2-oxoethyl acetate (900 mg, 3.308 mmol) and NaOH (266 mg, 6.616 mmol) in methanol (30 mL) was stirred for 2 h at room temperature. After consumption of starting material, the resulting mixture was concentrated under reduced pressure. The resulting mixture was diluted with water (10 mL). The mixture was acidified to pH 5 with 2 N aqueous HCl. The resulting solution was extracted with ethyl acetate (3×30 mL). The organic phases were combined, washed with brine and dried over Na₂SO₄. The solvent was concentrated under reduced pressure and the residue was purified by flash chromatography eluting with ethyl acetate in petroleum ether (0% to 30% gradient) to yield the title compound (630 mg, 82%). LC-MS (M+H)⁺=230.1.

### Step 2: 3-(4-bromophenyl)oxazolidin-4-one

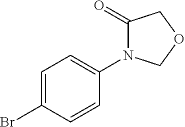 [SMILES: O=C1COCN1c1ccc(Br)cc1]

To a solution of N-(4-bromophenyl)-2-hydroxyacetamide (400 mg, 1.737 mmol) and CH₂Br₂ (604 mg, 3.474 mmol) in N,N-dimethylformamide (20 mL) was added LiHMDS (1.9 mL, 3.800 mmol, 2M in THF) dropwise at 0° C. under nitrogen atmosphere. The resulting mixture was stirred for 2 h at 110° C. under nitrogen atmosphere. After consumption of starting material, the resulting mixture was concentrated under reduced pressure. The residue was applied onto C18 gel column and purified by flash chromatography eluting with MeCN in water (with 10 mmol/L NH₄HCO₃) 0% to 70% gradient in 30 min to yield the title compound (80 mg, 19%). LC-MS (M+H)⁺=242.1.

### Step 3: 3-(4-(4,4,5,5-tetramethyl-1,3,2-dioxaborolan-2-yl)phenyl)oxazolidin-4-one

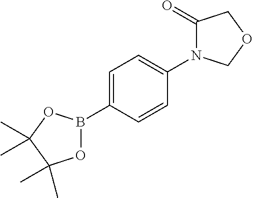 [SMILES: CC1(C)OB(c2ccc(N3COCC3=O)cc2)OC1(C)C]

The title compound (45 mg, 47%) was prepared in a manner similar to that in Example 28 step 2 from 3-(4-bromophenyl)oxazolidin-4-one. LC-MS (M+H)⁺=290.0.

### Step 4: 5-(2-methoxyphenyl)-N-(5-(4-(4-oxooxazolidin-3-yl)phenyl)thiazolo[5,4-b]pyridin-2-yl)pyridazine-4-carboxamide

The title compound (5 mg, 6%) was prepared in a manner similar to that in Example 78 step 3 from 3-(4-(4,4,5,5-tetramethyl-1,3,2-dioxaborolan-2-yl)phenyl)oxazolidin-4-one and N-(5-bromothiazolo[5,4-b]pyridin-2-yl)-5-(2-methoxyphenyl)pyridazine-4-carboxamide. ¹H NMR (400 MHz, d6-DMSO) δ 13.28 (brs, 1H), 9.50 (s, 1H), 9.40 (s, 1H), 8.24-8.05 (m, 4H), 7.79-7.70 (m, 2H), 7.56-7.46 (m, 2H), 7.19-7.05 (m, 2H), 5.60 (s, 2H), 4.44 (s, 2H), 3.56 (s, 3H). LC-MS (M+H)⁺=525.2.

### Example 150: N-(5-(2,4-dichloro-6-methylphenyl)thiazolo[5,4-b]pyridin-2-yl)-5-(2-methoxyphenyl)pyridazine-4-carboxamide

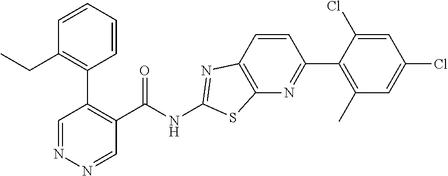 [SMILES: CCc1ccccc1-c1cnncc1C(=O)Nc1nc2ccc(-c3c(C)cc(Cl)cc3Cl)nc2s1]

### Step 1: 2-(2,4-dichloro-6-methylphenyl)-4,4,5,5-tetramethyl-1,3,2-dioxaborolane

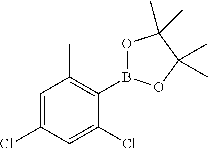 [SMILES: Cc1cc(Cl)cc(Cl)c1B1OC(C)(C)C(C)(C)O1]

The title compound (285 mg, 66%) was prepared in a manner similar to that in Example 87 step 2 from 1,5-dichloro-2-iodo-3-methylbenzene. 1H NMR (400 MHz, d-CDCl3) δ 7.16 (s, 1H), 7.03 (s, 1H), 2.36 (s, 3H), 1.40 (s, 12H).

### Step 2: N-(5-(2,4-dichloro-6-methylphenyl)thiazolo[5,4-b]pyridin-2-yl)-5-(2-methoxyphenyl)pyridazine-4-carboxamide

The title compound (58 mg, 23%) was prepared in a manner similar to that in Example 78 step 3 from N-(5-bromothiazolo[5,4-b]pyridin-2-yl)-5-(2-methoxyphenyl)pyridazine-4-carboxamide and 2-(2,4-dichloro-6-methylphenyl)-4,4,5,5-tetramethyl-1,3,2-dioxaborolane. 1H NMR (400 MHz, d6-DMSO) δ 13.35 (s, 1H), 9.50 (s, 1H), 9.43 (s, 1H), 8.27 (d, J=8.3 Hz, 1H), 7.60 (s, 1H), 7.58-7.43 (m, 4H), 7.15 (t, J=7.4 Hz, 1H), 7.08 (d, J=8.3 Hz, 1H), 3.58 (s, 3H), 2.05 (s, 3H). LC-MS (M+H)⁺=522.3.

### Example 151: N-(5-(4-(azetidine-1-carbonyl)-3-methylphenyl)thiazolo[5,4-b]pyridin-2-yl)-4-(5-cyano-2-methoxyphenyl)-6-methylnicotinamide

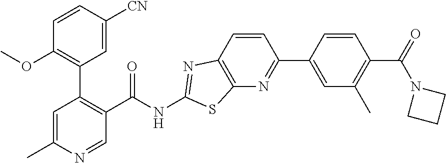 [SMILES: COc1ccc(C#N)cc1-c1cc(C)ncc1C(=O)Nc1nc2ccc(-c3ccc(C(=O)N4CCC4)c(C)c3)nc2s1]

### Step 1: methyl 4-(2-(4-(5-cyano-2-methoxyphenyl)-6-methylnicotinamido)thiazolo[5,4-b]pyridin-5-yl)-2-methylbenzoate

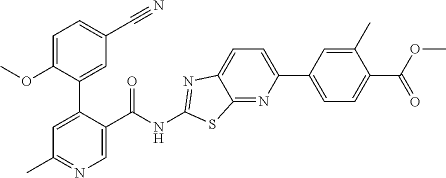

The title compound (300 mg, 75%) was prepared in a manner similar to that in Example 78 step 3 from N-(5-bromothiazolo[5,4-b]pyridin-2-yl)-4-(5-cyano-2-methoxyphenyl)-6-methylnicotinamide and (4-(methoxycarbonyl)-3-methylphenyl)boronic acid.

### Step 2: 4-(2-(4-(5-cyano-2-methoxyphenyl)-6-methylnicotinamido)thiazolo[5,4-b]pyridin-5-yl)-2-methylbenzoic acid

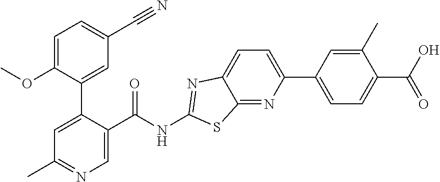 [SMILES: COc1ccc(C#N)cc1-c1cc(C)ncc1C(=O)Nc1nc2ccc(-c3ccc(C(=O)O)c(C)c3)nc2s1]

The title compound (200 mg, 68%) was prepared in a manner similar to that in Example 1 step 3 from methyl 4-(2-(4-(5-cyano-2-methoxyphenyl)-6-methylnicotinamido)thiazolo[5,4-b]pyridin-5-yl)-2-methylbenzoate. LC-MS (M+H)⁺=536.1.

### Step 3: N-(5-(4-(azetidine-1-carbonyl)-3-methylphenyl)thiazolo[5,4-b]pyridin-2-yl)-4-(5-cyano-2-methoxyphenyl)-6-methylnicotinamide

The title compound (11 mg, 10%) was prepared in a manner similar to that in Example 73 step 5 from 4-(2-(4-(5-cyano-2-methoxyphenyl)-6-methylnicotinamido)thiazolo[5,4-b]pyridin-5-yl)-2-methylbenzoic acid and azetidine. 1H NMR (400 MHz, d6-DMSO) δ 13.03 (s, 1H), 8.81 (s, 1H), 8.21 (d, J=8.3 Hz, 1H), 8.11 (d, J=8.5 Hz, 1H), 8.04 (s, 1H), 8.00-7.94 (m, 1H), 7.92 (s, 2H), 7.46 (s, 1H), 7.39 (d, J=8.0 Hz, 1H), 7.19 (d, J=9.2 Hz, 1H), 4.05 (t, J=7.6 Hz, 2H), 3.93 (t, J=7.5 Hz, 2H), 3.60 (s, 3H), 2.61 (s, 3H), 2.40 (s, 3H), 2.28-2.16 (m, 2H). LC-MS (M+H)⁺=575.9.

### Example 152: N-(5-(4-cyanophenoxy)thiazolo[5,4-b]pyridin-2-yl)-5-(2-methoxyphenyl)pyridazine-4-carboxamide

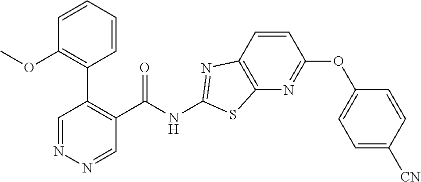 [SMILES: COc1ccccc1-c1cnncc1C(=O)Nc1nc2ccc(Oc3ccc(C#N)cc3)nc2s1]

### Step 1: tert-butyl (5-(4-cyanophenoxy)thiazolo[5,4-b]pyridin-2-yl)carbamate

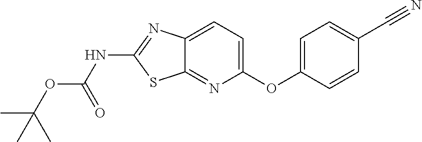 [SMILES: CC(C)(C)OC(=O)Nc1nc2ccc(Oc3ccc(C#N)cc3)nc2s1]

The title compound (1.1 g, 97%) was prepared in a manner similar to that in Example 129 step 1 from tert-butyl (5-bromothiazolo[5,4-b]pyridin-2-yl)carbamate and 4-hydroxybenzonitrile. LC-MS (M+H)⁺=369.0.

### Step 2: 4-((2-aminothiazolo[5,4-b]pyridin-5-yl)oxy)benzonitrile

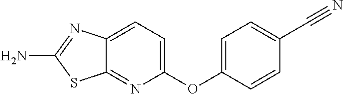 [SMILES: N#Cc1ccc(Oc2ccc3nc(N)sc3n2)cc1]

The title compound (202 mg, 92%) was prepared in a manner similar to that in Example 1 step 6 from tert-butyl (5-(4-cyanophenoxy)thiazolo[5,4-b]pyridin-2-yl)carbamate. LC-MS (M+H)⁺=269.0.

### Step 3: N-(5-(4-cyanophenoxy)thiazolo[5,4-b]pyridin-2-yl)-5-(2-methoxyphenyl)pyridazine-4-carboxamide

The title compound (5.2 mg, 12%) was prepared in a manner similar to that in Example 11 step 3 from 4-((2-aminothiazolo[5,4-b]pyridin-5-yl)oxy)benzonitrile and 5-(2-methoxyphenyl)pyridazine-4-carboxylic acid. ¹H NMR (400 MHz, d6-DMSO) δ: 13.24 (s, 1H), 9.47 (s, 1H), 9.41 (s, 1H), 8.33-8.22 (m, 1H), 7.95-7.86 (m, 2H), 7.55-7.49 (m, 2H), 7.49-7.45 (s, 2H), 7.30-7.26 (m, 1H), 7.15-7.10 (m, 1H), 7.09-7.03 (m, 1H), 3.53 (s, 3H). LC-MS (M+H)⁺=481.4.

### Example 153: 4-(2-cyano-6-methoxyphenyl)-N-(5-(4-cyanophenyl)thiazolo[5,4-b]pyridin-2-yl)-6-methylnicotinamide

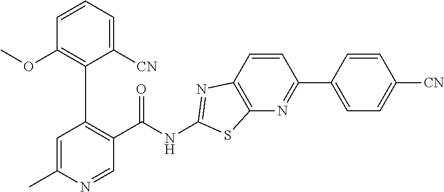 [SMILES: COc1cccc(C#N)c1-c1cc(C)ncc1C(=O)Nc1nc2ccc(-c3ccc(C#N)cc3)nc2s1]

### Step 1: methyl 4-(2-cyano-6-methoxyphenyl)-6-methylnicotinate

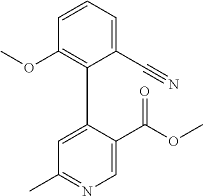

The title compound (293 mg, 82%) was prepared in a manner similar to that described in Example 2 step 2 from methyl 4-chloro-6-methylnicotinate and 3-methoxy-2-(4,4,5,5-tetramethyl-1,3,2-dioxaborolan-2-yl)benzonitrile. LC-MS (M+H)⁺=283.1.

### Step 2: 4-(2-cyano-6-methoxyphenyl)-6-methylnicotinic acid

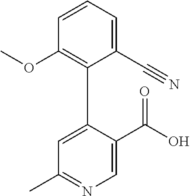 [SMILES: COc1cccc(C#N)c1-c1cc(C)ncc1C(=O)O]

The title compound (210 mg, 82%) was prepared in a manner similar to that described in Example 1 step 3 from methyl 4-(2-cyano-6-methoxyphenyl)-6-methylnicotinate. LC-MS (M+H)⁺=269.0.

### Step 3: 4-(2-cyano-6-methoxyphenyl)-N-(5-(4-cyanophenyl)thiazolo[5,4-b]pyridin-2-yl)-6-methylnicotinamide

The title compound (6.8 mg, 5.5%) was prepared in a manner similar to that described in Example 11 step 3 from 4-(2-cyano-6-methoxyphenyl)-6-methylnicotinic acid and 4-(2-aminothiazolo[5,4-b]pyridin-5-yl)benzonitrile. ¹H NMR (400 MHz, d6-DMSO) δ:13.27 (s, 1H), 8.97 (s, 1H), 8.35-8.28 (m, 2H), 8.26-8.21 (m, 2H), 8.02-7.89 (m, 2H), 7.65-7.58 (m, 2H), 7.55-7.45 (m, 1H), 7.40 (d, 8.0 Hz, 1H), 3.60 (s, 3H), 2.62 (s, 3H). LC-MS (M+H)⁺=503.1.

### Example 154: N-(5-(4-chloro-2-methoxyphenoxy)thiazolo[5,4-b]pyridin-2-yl)-5-(2-methoxyphenyl)pyridazine-4-carboxamide

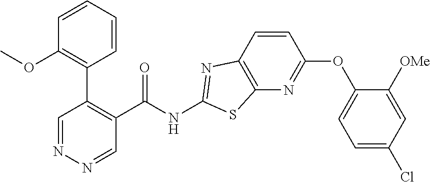 [SMILES: COc1cc(Cl)ccc1Oc1ccc2nc(NC(=O)c3cnncc3-c3ccccc3OC)sc2n1]

The title compound (25 mg, 21%) was prepared in a manner similar to that in Example 129 from N-(5-bromothiazolo[5,4-b]pyridin-2-yl)-5-(2-methoxyphenyl)pyridazine-4-carboxamide and 4-chloro-2-methoxyphenol. ¹H NMR (400 MHz, d6-DMSO) δ 13.13 (s, 1H), 9.45 (s, 1H), 9.41 (s, 1H), 8.19 (d, J=8.7 Hz, 1H), 7.49 (dd, J=19.7, 7.7 Hz, 2H), 7.25 (dd, J=13.9, 5.1 Hz, 2H), 7.13 (dd, J=8.1, 5.1 Hz, 2H), 7.06 (dd, J=11.8, 5.0 Hz, 2H), 3.72 (s, 3H), 3.53 (s, 3H). LC-MS (M+H)⁺=520.3.

### Example 155: N-(5-(4-chloro-2-fluorophenoxy)thiazolo[5,4-b]pyridin-2-yl)-5-(2-methoxyphenyl)pyridazine-4-carboxamide

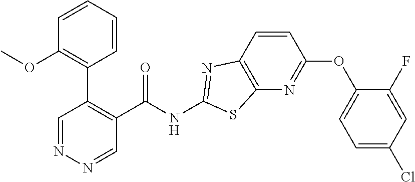 [SMILES: COc1ccccc1-c1cnncc1C(=O)Nc1nc2ccc(Oc3ccc(Cl)cc3F)nc2s1]

The title compound (14 mg, 12%) was prepared in a manner similar to that in Example 129 from N-(5-bromothiazolo[5,4-b]pyridin-2-yl)-5-(2-methoxyphenyl)pyridazine-4-carboxamide and 4-chloro-2-fluorophenol. ¹H NMR (400 MHz, d6-DMSO) δ 13.19 (s, 1H), 9.46 (s, 1H), 9.41 (s, 1H), 8.26 (d, J=8.7 Hz, 1H), 7.67 (dd, J=10.5, 1.8 Hz, 1H), 7.56-7.42 (m, 3H), 7.37 (d, J=8.7 Hz, 1H), 7.29 (d, J=8.7 Hz, 1H), 7.13 (t, J=7.5 Hz, 1H), 7.04 (d, J=8.4 Hz, 1H), 3.52 (s, 3H). LC-MS (M+H)⁺=508.2.

### Example 156: N-(5-(4-cyanophenyl)thiazolo[5,4-b]pyridin-2-yl)-4-(2-methoxy-5-nitrophenyl)nicotinamide

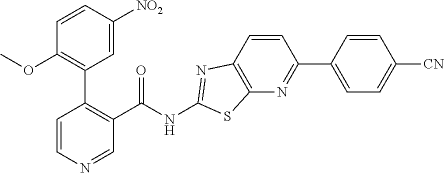 [SMILES: COc1ccc([N+](=O)[O-])cc1-c1ccncc1C(=O)Nc1nc2ccc(-c3ccc(C#N)cc3)nc2s1]

### Step 1: methyl 4-(2-methoxy-5-nitrophenyl)nicotinate

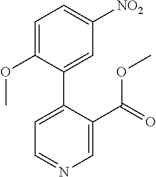 [SMILES: COC(=O)c1cnccc1-c1cc([N+](=O)[O-])ccc1OC]

The title compound (210 mg, 90%) was prepared in a manner similar to Example 2 of Step 2 from 2-(2-methoxy-5-nitrophenyl)-5,5-dimethyl-1,3,2-dioxaborinane and methyl 4-chloronicotinate. LC-MS (M+H)⁺=289.3.

### Step 2: 4-(2-methoxy-5-nitrophenyl)nicotinic acid

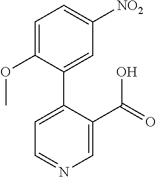 [SMILES: COc1ccc([N+](=O)[O-])cc1-c1ccncc1C(=O)O]

The title compound (85 mg, 42%) was prepared in a manner similar to Example 1 of Step 3 from methyl 4-(2-methoxy-5-nitrophenyl)nicotinate. LC-MS (M+H)⁺=275.3.

### Step 3: N-(5-(4-cyanophenyl)thiazolo[5,4-b]pyridin-2-yl)-4-(2-methoxy-5-nitrophenyl)nicotinamide

The title compound (20 mg, 15%) was prepared in a manner similar to Example 11 of Step 3 from 4-(2-methoxy-5-nitrophenyl)nicotinic acid and 4-(2-aminothiazolo[5,4-b]pyridin-5-yl)benzonitrile. 1H NMR (400 MHz, d6-DMSO) δ 13.19 (s, 1H), 8.97 (s, 1H), 8.88 (d, J=5.0 Hz, 1H), 8.46-8.18 (m, 6H), 7.98 (d, J=8.2 Hz, 2H), 7.64 (d, J=5.0 Hz, 1H), 7.25 (d, J=9.2 Hz, 1H), 3.68 (s, 3H). LC-MS (M+H)⁺=509.4.

### Example 157: N-(5-(4-cyanophenyl)thiazolo[5,4-b]pyridin-2-yl)-4-(2-cyclopropyl-5-(methylsulfonyl)phenyl)-6-methylnicotinamide

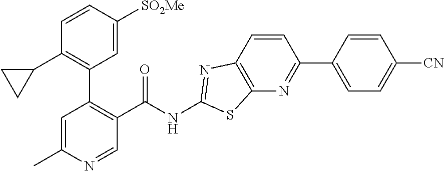 [SMILES: Cc1cc(-c2cc(S(C)(=O)=O)ccc2C2CC2)c(C(=O)Nc2nc3ccc(-c4ccc(C#N)cc4)nc3s2)cn1]

### Step 1: (4-bromo-3-chlorophenyl)(methyl)sulfane

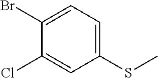

To a solution of 1-bromo-2-chloro-4-fluorobenzene (1.0 g, 4.7 mmol) in DMSO (20 mL) was added NaSCH₃ (500 mg, 7.1 mmol). The mixture was stirred at 140° C. for 16 h. The mixture was cooled and diluted with water (50 mL), extracted with ethyl acetate (3×30 mL). The combined organic layers were washed with brine (30 mL). dried over Na₂SO₄, filtered and concentrated under vacuum. The residue was purified by silica gel column, eluting with petroleum ether to give the title compound (1.0 g, 90%). 1H NMR (400 MHz, cdcl3) δ 7.48 (d, J=8.4 Hz, 1H), 7.29 (d, J=2.2 Hz, 1H), 6.98 (dd, J=8.4, 2.2 Hz, 1H), 2.47 (s, 3H).

### Step 2: 1-bromo-2-chloro-4-(methylsulfonyl)benzene

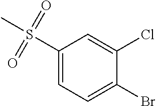

To a solution of (4-bromo-3-chlorophenyl)(methyl)sulfane (1.4 g, 5.9 mmol) in dichloromethane (20 mL) was added 3-chloroperoxybenzoic acid (4.0 g, 17.4 mmol) at 0° C. in portions. The mixture was stirred at room temperature for 14 h. The mixture was filtered, the filtrate was washed with aqueous K₂CO₃ solution (0.5 M, 20 mL), dried over Na₂SO₄, concentrated under vacuum to give the title compound (1.4 g, crude). LC-MS (M+H)⁺=268.9, 270.9.

### Step 3: 2-chloro-1-cyclopropyl-4-(methylsulfonyl)benzene

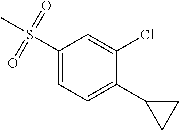

To a mixture of 1-bromo-2-chloro-4-(methylsulfonyl)benzene (1.4 g, 5.2 mmol), cyclopropylboronic acid (540 mg, 6.3 mmol) and K₃PO₄ (3.3 g, 15.5 mmol) in toluene (30 mL) and water (10 mL) was added PdCl₂[P(Cy)₃]₂ (200 mg, 0.3 mmol). The mixture was stirred at 100° C. for 16 h under nitrogen atmosphere. The mixture was cooled, extracted with ethyl acetate (30 mL). The organic layers were washed with brine (30 mL). dried over Na₂SO₄, filtered and concentrated under vacuum. The residue was purified by silica gel column, eluting with ethyl acetate in petroleum ether (20%, v/v) to give title compound (0.9 g, 75%). LC-MS (M+H)⁺=231.0, 233.0.

### Step 4: 2-(2-cyclopropyl-5-(methylsulfonyl)phenyl)-4,4,5,5-tetramethyl-1,3,2-dioxaborolane

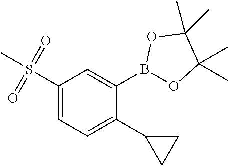

To a mixture of 2-chloro-1-cyclopropyl-4-(methylsulfonyl)benzene (900 mg, 3.9 mmol) and 4,4,4′,4′,5,5,5′,5′-octamethyl-2,2′-bi(1,3,2-dioxaborolane) (2.0 g, 7.9 mmol) in dioxane (50 mL) was added KOAc (1.8 g, 18.4 mmol) and XPhos Pd G3 (200 mg, 0.25 mmol). The mixture was stirred at 100° C. for 16 h. The mixture was cooled and filtered. The filtrate was concentrated under vacuum, purified by gel column, eluting with ethyl acetate in petroleum ether (17%, v/v) to give the title compound (250 mg, 20%). LC-MS (M+H)⁺=323.2.

### Step 5: methyl 4-(2-cyclopropyl-5-(methylsulfonyl)phenyl)-6-methylnicotinate

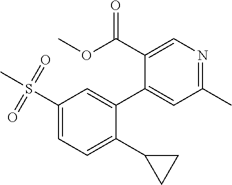

The title compound (200 mg, 72%) was prepared in a manner similar to that in Example 2 step 2 from 2-(2-cyclopropyl-5-(methylsulfonyl)phenyl)-4,4,5,5-tetramethyl-1,3,2-dioxaborolane and methyl 4-chloro-6-methylnicotinate. LC-MS (M+H)⁺=346.2.

### Step 6: 4-(2-cyclopropyl-5-(methylsulfonyl)phenyl)-6-methylnicotinic acid

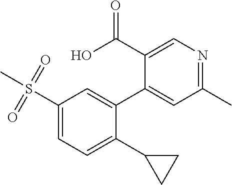

The title compound (100 mg, 50%) was prepared in a manner similar to that described in Example 1 step 3 from methyl 4-(2-cyclopropyl-5-(methylsulfonyl)phenyl)-6-methylnicotinate. LC-MS (M+H)⁺=332.2.

### Step 7: N-(5-(4-cyanophenyl)thiazolo[5,4-b]pyridin-2-yl)-4-(2-cyclopropyl-5-(methylsulfonyl)phenyl)-6-methylnicotinamide

The title compound (60 mg, 33%) was prepared in a manner similar to that described in Example 11 step 3 from 4-(2-cyclopropyl-5-(methylsulfonyl)phenyl)-6-methylnicotinic acid and 4-(2-aminothiazolo[5,4-b]pyridin-5-yl)benzonitrile. ¹H NMR (400 MHz, d6-DMSO) δ 13.16 (s, 1H), 8.93 (s, 1H), 8.33 (d, J=8.2 Hz, 2H), 8.28-8.20 (m, 2H), 7.90 (d, J=8.2 Hz, 2H), 7.83 (d, J=8.0 Hz, 1H), 7.73 (s, 1H), 7.46 (s, 1H), 7.12 (d, J=8.0 Hz, 1H), 3.20 (s, 3H), 2.64 (s, 3H), 1.70-1.58 (m, 1H), 0.95-0.70 (m, 3H), 0.70-0.55 (m, 1H). LC-MS (M+H)⁺=566.5.

### Example 158: N-(5-(5-cyanopyridin-2-yl)thiazolo[5,4-b]pyridin-2-yl)-5-(2-methoxyphenyl)pyridazine-4-carboxamide

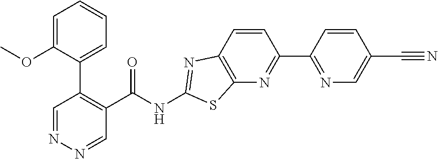 [SMILES: COc1ccccc1-c1cnncc1C(=O)Nc1nc2ccc(-c3ccc(C#N)cn3)nc2s1]

To a solution of N-(5-bromothiazolo[5,4-b]pyridin-2-yl)-5-(2-methoxyphenyl)pyridazine-4-carboxamide (100 mg, 0.226 mmol) and 6-(tributylstannyl)nicotinonitrile (98 mg, 0.249 mmol) in N,N-dimethylformamide (3 mL) was added Pd(PPh₃)₂Cl₂ (16 mg, 0.0226 mmol), CuI (4.3 mg, 0.0226 mmol). The mixture was stirred at 115° C. for 18 h under nitrogen. The reaction mixture was cooled to room temperature and diluted with ethyl acetate (20 mL), washed with brine (20 mL), dried with over sodium sulfate, and concentrated under reduced pressure. The residue was purified by silica gel column chromatography, eluting with methanol in dichloromethane (5%, v/v) to afford the title compound (33.56 mg, 32%). ¹H NMR (400 MHz, d6-DMSO) δ 13.47 (s, 1H), 9.51 (s, 1H), 9.43 (s, 1H), 9.15 (s, 1H), 8.58 (d, J=8.0 Hz, 2H), 8.45 (dd, J=8.4, 1.4 Hz, 1H), 8.32 (d, J=8.3 Hz, 1H), 7.62-7.43 (m, 2H), 7.16 (t, J=7.5 Hz, 1H), 7.07 (d, J=8.3 Hz, 1H), 3.55 (s, 3H). LC-MS (M+H)⁺=466.4.

### Example 159: 4-(5-cyano-2-methoxyphenyl)-N-(5-(5-cyanopyridin-2-yl)thiazolo[5,4-b]pyridin-2-yl)-6-methylnicotinamide

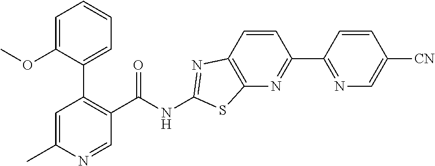 [SMILES: COc1ccccc1-c1cc(C)ncc1C(=O)Nc1nc2ccc(-c3ccc(C#N)cn3)nc2s1]

To a solution of N-(5-bromothiazolo[5,4-b]pyridin-2-yl)-4-(5-cyano-2-methoxyphenyl)-6-methylnicotinamide (109 mg, 0.226 mmol) and 6-(tributylstannyl)nicotinonitrile (98 mg, 0.249 mmol) in N,N-dimethylformamide (3 mL) was added Pd(PPh₃)₂Cl₂ (16 mg, 0.0226 mmol), CuI (4.3 mg, 0.0226 mmol). The mixture was stirred at 115° C. for 18 h under nitrogen. The reaction mixture was cooled to room temperature and diluted with ethyl acetate (20 mL), washed with brine (20 mL), dried with over sodium sulfate, and concentrated under reduced pressure. The residue was purified by silica gel column chromatography, eluting with methanol in dichloromethane (5%, v/v) to afford the title compound (13.76 mg, 12%). ¹H NMR (400 MHz, d6-DMSO) δ:13.16 (s, 1H), 9.13 (s, 1H), 8.84 (s, 1H), 8.56 (t, J=9.0 Hz, 2H), 8.43 (d, J=8.3 Hz, 1H), 8.25 (s, 1H), 7.91 (d, J=8.8 Hz, 2H), 7.42 (s, 1H), 7.18 (d, J=8.5 Hz, 1H), 3.60 (s, 3H), 2.60 (s, 3H). LC-MS (M+H)⁺=504.4.

### Example 160: 5-(2-methoxyphenyl)-N-(5-(4-(methylsulfonyl)phenoxy)thiazolo[5,4-b]pyridin-2-yl)pyridazine-4-carboxamide

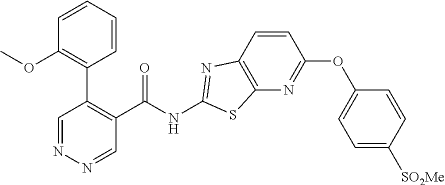 [SMILES: COc1ccccc1-c1cnncc1C(=O)Nc1nc2ccc(Oc3ccc(S(C)(=O)=O)cc3)nc2s1]

### Step 1: tert-butyl (5-(4-(methylsulfonyl)phenoxy)thiazolo[5,4-b]pyridin-2-yl)carbamate

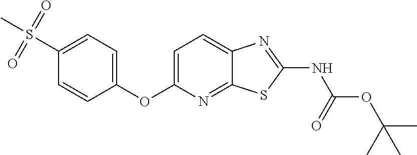 [SMILES: CC(C)(C)OC(=O)Nc1nc2ccc(Oc3ccc(S(C)(=O)=O)cc3)nc2s1]

The title compound (180 mg, 35%) was prepared in a manner similar to that described in Example 129 step 1 from tert-butyl (5-bromothiazolo[5,4-b]pyridin-2-yl)carbamate and 4-(methylsulfonyl)phenol. LC-MS (M+H)⁺=422.1.

### Step 2: 5-(4-(methylsulfonyl)phenoxy)thiazolo[5,4-b]pyridin-2-amine

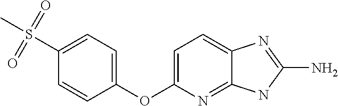 [SMILES: CS(=O)(=O)c1ccc(Oc2ccc3nc(N)[nH]c3n2)cc1]

The title compound (90 mg, 85%) was prepared in a manner similar to that described in Example 1 step 6 from tert-butyl (5-(4-(methylsulfonyl)phenoxy)thiazolo[5,4-b]pyridin-2-yl)carbamate. LC-MS (M+H)⁺=322.0.

### Step 3: 5-(2-methoxyphenyl)-N-(5-(4-(methylsulfonyl)phenoxy)thiazolo[5,4-b]pyridin-2-yl)pyridazine-4-carboxamide

The title compound (6.5 mg, 9.5%) was prepared in a manner similar to that described in Example 11 step 3 from 5-(4-(methylsulfonyl)phenoxy)thiazolo[5,4-b]pyridin-2-amine and 5-(2-methoxyphenyl)pyridazine-4-carboxylic acid. ¹H NMR (400 MHz, d6-DMSO) δ: 13.23 (s, 1H), 9.47 (s, 1H), 9.40 (s, 1H), 8.33-8.22 (m, 1H), 7.98-7.75 (m, 2H), 7.57-7.44 (s, 4H), 7.30-7.26 (m, 1H), 7.15-7.10 (m, 1H), 7.09-7.03 (m, 1H), 3.54 (s, 3H), 3.25 (s, 3H). LC-MS (M+H)⁺=534.4.

### Example 161: 4-(5-cyano-2-methoxyphenyl)-N-(5-(4-cyano-3-methoxyphenyl)thiazolo[5,4-b]pyridin-2-yl)-6-methylnicotinamide

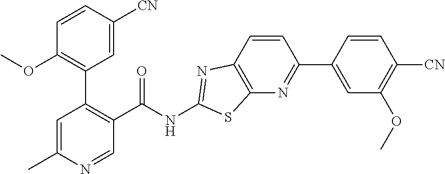 [SMILES: COc1cc(-c2ccc3nc(NC(=O)c4cnc(C)cc4-c4cc(C#N)ccc4OC)sc3n2)ccc1C#N]

The title compound (18 mg, 11%) was prepared in a manner similar to that described in Example 78 step 3 from 2-methoxy-4-(4,4,5,5-tetramethyl-1,3,2-dioxaborolan-2-yl)benzonitrile. ¹H NMR (400 MHz, d6-DMSO) δ: 13.09 (s, 1H), 8.82 (s, 1H), 8.30-8.19 (m, 2H), 8.00-7.78 (m, 5H), 7.47-7.41 (s, 1H), 7.23-7.15 (m, 1H), 4.06 (s, 3H), 3.59 (s, 3H), 2.60 (s, 3H). LC-MS (M+H)⁺=533.1.

### Example 162: N-(5-(2-(5-chloropyridin-2-yl)ethyl)thiazolo[5,4-b]pyridin-2-yl)-5-(2-methoxyphenyl)pyridazine-4-carboxamide

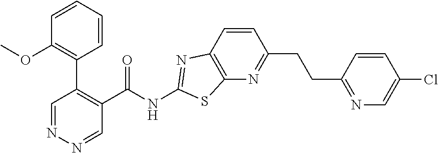 [SMILES: COc1ccccc1-c1cnncc1C(=O)Nc1nc2ccc(CCc3ccc(Cl)cn3)nc2s1]

### Step 1: tert-butyl (5-((5-chloropyridin-2-yl)ethynyl)thiazolo[5,4-b]pyridin-2-yl)carbamate

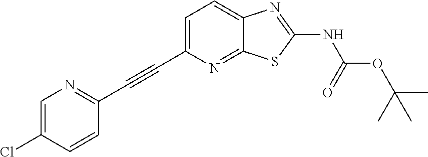 [SMILES: CC(C)(C)OC(=O)Nc1nc2ccc(C#Cc3ccc(Cl)cn3)nc2s1]

A solution of tert-butyl (5-bromothiazolo[5,4-b]pyridin-2-yl)carbamate (329 mg, 1.0 mmol), 5-chloro-2-ethynylpyridine (138 mg, 1.0 mmol) and Pd(PPh₃)₄ (120 mg, 0.1 mmol) in triethylamine (10 mL) was stirred for 16 h at 80° C. under nitrogen atmosphere. After consumption of starting material, the resulting mixture was concentrated under reduced pressure. The residue was applied onto C18 gel column and purified by flash chromatography eluting with MeCN in water (with 10 mmol/L NH₄HCO₃) 40% to 80% gradient in 30 min to yield the title compound (180 mg, 46%). LC-MS (M+H)⁺=387.0.

### Step 2: tert-butyl (5-(2-(5-chloropyridin-2-yl)ethyl)thiazolo[5,4-b]pyridin-2-yl)carbamate

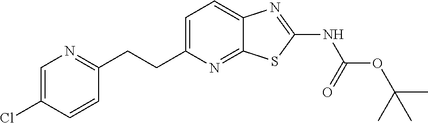 [SMILES: CC(C)(C)OC(=O)Nc1nc2ccc(CCc3ccc(Cl)cn3)nc2s1]

The title compound (45 mg, 17%) was prepared in a manner similar to that in Example 67 step 2 from tert-butyl (5-((5-chloropyridin-2-yl)ethynyl)thiazolo[5,4-b]pyridin-2-yl)carbamate. LC-MS (M+H)⁺=391.1.

### Step 3: 5-(2-(5-chloropyridin-2-yl)ethyl)thiazolo[5,4-b]pyridin-2-amine

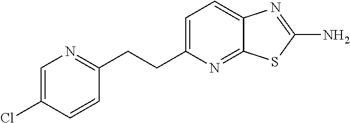 [SMILES: Nc1nc2ccc(CCc3ccc(Cl)cn3)nc2s1]

The title compound (29 mg, 86%) was prepared in a manner similar to that in Example 1 step 6 from tert-butyl (5-(2-(5-chloropyridin-2-yl)ethyl)thiazolo[5,4-b]pyridin-2-yl)carbamate. LC-MS (M+H)⁺=291.1.

### Step 4: N-(5-(2-(5-chloropyridin-2-yl)ethyl)thiazolo[5,4-b]pyridin-2-yl)-5-(2-methoxyphenyl)pyridazine-4-carboxamide

The title compound (8 mg, 18%) was prepared in a manner similar to that in Example 11 step 3 from 5-(2-(5-chloropyridin-2-yl)ethyl)thiazolo[5,4-b]pyridin-2-amine and 5-(2-methoxyphenyl)pyridazine-4-carboxylic acid. ¹H NMR (300 MHz, d6-DMSO) δ 9.47 (s, 1H), 9.38 (s, 1H), 8.57-8.50 (m, 1H), 8.03-7.94 (m, 1H), 7.86-7.77 (m, 1H), 7.55-7.42 (m, 2H), 7.40-7.30 (m, 2H), 7.19-7.02 (m, 2H), 3.45-3.35 (m, 2H), 3.31-3.15 (m, 5H). LC-MS (M+H)⁺=503.1.

### Example 163: 4-(5-cyano-2-methoxyphenyl)-N-(5-(4-(2,4-dioxooxazolidin-3-yl)-3-methylphenyl)thiazolo[5,4-b]pyridin-2-yl)-6-methylnicotinamide

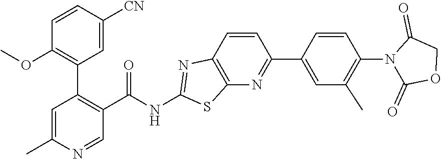 [SMILES: COc1ccc(C#N)cc1-c1cc(C)ncc1C(=O)Nc1nc2ccc(-c3ccc(N4C(=O)COC4=O)c(C)c3)nc2s1]

### Step 1: 3-(4-bromo-2-methylphenyl)oxazolidine-2,4-dione

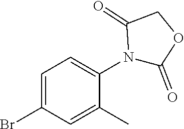

To a solution of 4-bromo-1-isocyanato-2-methylbenzene (200 mg, 0.945 mmol) and 1,4-dioxane-2,5-dione (55 mg, 0.475 mmol) in MeCN (1 mL, 95%) was added dibutyldimethoxystannane (29 mg, 0.097 mmol) dropwise at room temperature under nitrogen atmosphere. The resulting mixture was irradiated with microwave radiation for 20 min at 120° C. After consumption of starting material, the reaction was then quenched by the addition of water (10 mL). the resulting mixture was concentrated under reduced pressure. The resulting mixture was diluted with water (10 mL). The resulting mixture was extracted with ethyl acetate (2×25 mL). The organic phases were combined, washed with brine and dried over Na₂SO₄. The solvent was concentrated under reduced pressure and the residue was purified by flash chromatography eluting with ethyl acetate in petroleum ether (0% to 50% gradient) to yield the title compound (160 mg, 62%). ¹H NMR (400 MHz, d6-DMSO) δ 7.68-7.63 (m, 1H), 7.60-7.53 (m, 1H), 7.30 (d, J=8.4 Hz, 1H), 5.16-4.92 (m, 2H), 2.17 (s, 3H).

### Step 2: 3-(2-methyl-4-(4,4,5,5-tetramethyl-1,3,2-dioxaborolan-2-yl)phenyl)oxazolidine-2,4-dione

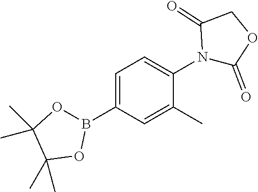 [SMILES: Cc1cc(B2OC(C)(C)C(C)(C)O2)ccc1N1C(=O)COC1=O]

The title compound (135 mg, 71%) was prepared in a manner similar to that in Example 28 step 2 from 3-(4-bromo-2-methylphenyl)oxazolidine-2,4-dione. ¹H NMR (400 MHz, d6-DMSO) δ 7.70-7.65 (m, 1H), 7.65-7.58 (m, 1H), 7.34 (d, J=7.8 Hz, 1H), 5.17-4.93 (m, 2H), 2.19 (s, 3H), 1.31 (s, 12H).

### Step 3: 4-(5-cyano-2-methoxyphenyl)-N-(5-(4-(2,4-dioxooxazolidin-3-yl)-3-methylphenyl)thiazolo[5,4-b]pyridin-2-yl)-6-methylnicotinamide

The title compound (4 mg, 8%) was prepared in a manner similar to that in Example 78 step 3 from 3-(2-methyl-4-(4,4,5,5-tetramethyl-1,3,2-dioxaborolan-2-yl)phenyl)oxazolidine-2,4-dione and N-(5-bromothiazolo[5,4-b]pyridin-2-yl)-4-(5-cyano-2-methoxyphenyl)-6-methylnicotinamide. ¹H NMR (400 MHz, d6-DMSO) δ 13.14 (brs, 1H), 8.91 (s, 1H), 8.28-8.04 (m, 4H), 7.98-7.92 (m, 2H), 7.62 (s, 1H), 7.50-7.44 (m, 1H), 7.25-7.18 (m, 1H), 5.22-4.96 (m, 2H), 3.61 (s, 3H), 2.66 (s, 3H), 2.27 (s, 3H). LC-MS (M+H)⁺=591.1.

### Example 164: 4-(5-cyano-2-methoxyphenyl)-6-methyl-N-(5-(6-(2-oxopyrrolidin-1-yl)pyridin-3-yl)thiazolo[5,4-b]pyridin-2-yl)nicotinamide

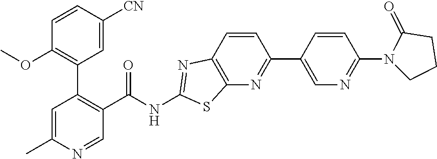 [SMILES: COc1ccc(C#N)cc1-c1cc(C)ncc1C(=O)Nc1nc2ccc(-c3ccc(N4CCCC4=O)nc3)nc2s1]

The title compound (8 mg, 7%) was prepared in a manner similar to that in Example 78 step 3 from 1-(5-(4,4,5,5-tetramethyl-1,3,2-dioxaborolan-2-yl)pyridin-2-yl)pyrrolidin-2-one and N-(5-bromothiazolo[5,4-b]pyridin-2-yl)-4-(5-cyano-2-methoxyphenyl)-6-methylnicotinamide. 1H NMR (400 MHz, d6-DMSO) δ 12.99 (s, 1H), 9.10 (s, 1H), 8.84 (s, 1H), 8.56-8.48 (m, 1H), 8.47-8.38 (m, 1H), 8.17-8.01 (m, 2H), 7.95-7.81 (m, 2H), 7.39 (s, 1H), 7.18 (d, J=8.7 Hz, 1H), 4.06 (t, J=7.1 Hz, 2H), 3.60 (s, 3H), 2.66-2.54 (m, 5H), 2.14-2.01 (m, 2H). LC-MS (M+H)⁺=562.3.

### Example 165: N-(5-(4-acetamido-3-methylphenyl)thiazolo[5,4-b]pyridin-2-yl)-5-(2-methoxyphenyl)pyridazine-4-carboxamide

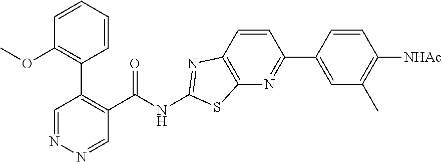 [SMILES: COc1ccccc1-c1cnncc1C(=O)Nc1nc2ccc(-c3ccc(NC(C)=O)c(C)c3)nc2s1]

### Step 1: N-(4-bromo-2-methylphenyl)acetamide

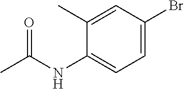

The title compound (2.2 g, 90%) was prepared in a manner similar to Example 140 step 1 from 4-bromo-2-methylaniline. LC-MS (M+H)⁺=227.9, 229.9.

### Step 2: N-(2-methyl-4-(4,4,5,5-tetramethyl-1,3,2-dioxaborolan-2-yl)phenyl)acetamide

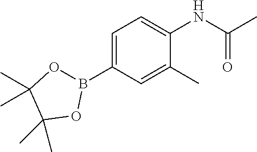 [SMILES: CC(=O)Nc1ccc(B2OC(C)(C)C(C)(C)O2)cc1C]

The title compound (0.6 g, 50%) was prepared in a manner similar to Example 28 step 2 from N-(4-bromo-2-methylphenyl)acetamide. LC-MS (M+H)⁺=276.1.

### Step 3: N-(5-(4-acetamido-3-methylphenyl)thiazolo[5,4-b]pyridin-2-yl)-5-(2-methoxyphenyl)pyridazine-4-carboxamide

The title compound (9 mg, 7.8%) was prepared in a manner similar to Example 96 step 2 from N-(5-bromothiazolo[5,4-b]pyridin-2-yl)-5-(2-methoxyphenyl)pyridazine-4-carboxamide and N-(2-methyl-4-(4,4,5,5-tetramethyl-1,3,2-dioxaborolan-2-yl)phenyl)acetamide. ¹H NMR (400 MHz, d6-DMSO) δ 13.27 (s, 1H), 9.49 (s, 1H), 9.41 (s, 1H), 9.35 (s, 1H), 8.15 (d, J=10.0 Hz, 1H), 8.05 (d, J=7.9 Hz, 1H), 7.99 (s, 1H), 7.92 (d, J=8.4 Hz, 1H), 7.63 (d, J=8.3 Hz, 1H), 7.53 (d, J=7.2 Hz, 1H), 7.49 (t, J=8.0 Hz, 1H), 7.15 (t, J=7.4 Hz, 1H), 7.07 (d, J=8.3 Hz, 1H), 3.56 (s, 3H), 2.31 (s, 3H), 2.10 (s, 3H). LC-MS (M+H)⁺=511.1.

### Example 166: 4-(5-cyano-2-methoxyphenyl)-N-(5-(4-cyano-3-methylphenyl)thiazolo[5,4-b]pyridin-2-yl)-6-methylnicotinamide

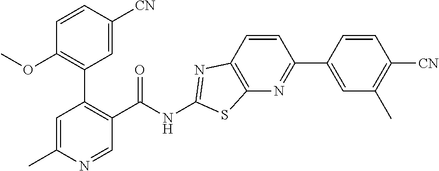 [SMILES: COc1ccc(C#N)cc1-c1cc(C)ncc1C(=O)Nc1nc2ccc(-c3ccc(C#N)c(C)c3)nc2s1]

The title compound (25 mg, 15%) was prepared in a manner similar to that in Example 78 step 3 from 2-methyl-4-(4,4,5,5-tetramethyl-1,3,2-dioxaborolan-2-yl)benzonitrile and N-(5-bromothiazolo[5,4-b]pyridin-2-yl)-4-(5-cyano-2-methoxyphenyl)-6-methylnicotinamide. ¹H NMR (400 MHz, d6-DMSO) δ: 13.18 (s, 1H), 8.81 (s, 1H), 8.30-8.18 (m, 3H), 8.16-8.10 (m, 1H), 8.00-7.81 (m, 3H), 7.47-7.41 (s, 1H), 7.23-7.15 (m, 1H), 3.60 (s, 3H), 2.60 (s, 3H), 2.50 (s, 3H). LC-MS (M+H)⁺=517.1.

### Example 167: 4-(5-cyano-2-cyclopropylphenyl)-N-(5-(4-cyanophenyl)thiazolo[5,4-b]pyridin-2-yl)-6-methylnicotinamide

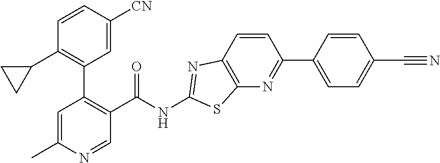 [SMILES: Cc1cc(-c2cc(C#N)ccc2C2CC2)c(C(=O)Nc2nc3ccc(-c4ccc(C#N)cc4)nc3s2)cn1]

### Step 1: Methyl 4-(2-chloro-5-cyanophenyl)-6-methylnicotinate

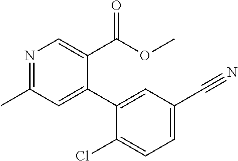 [SMILES: COC(=O)c1cnc(C)cc1-c1cc(C#N)ccc1Cl]

To a solution of methyl 4-chloro-6-methylnicotinate (1 g, 5.5 mmol) and (2-chloro-5-cyanophenyl)boronic acid (1.1 g, 6.1 mmol) in dioxane (10 mL) and water (2 mL) were added 1,1′-Bis(diphenylphosphino)ferrocene]dichloropalladium(II) (402 mg, 0.97 mmol) and K₂CO₃ (1.5 g, 11 mmol). The mixture was stirred at 100° C. for 4 h under nitrogen. The reaction mixture was cooled to room temperature and diluted with ethyl acetate (20 mL). The solution was washed with brine (20 mL), dried with over sodium sulfate, filtered and concentrated under reduced pressure. The residue was purified by silica gel column chromatography eluted with ethyl acetate in petroleum ether (30%, v/v) to afford the title compound (1.1 g, 70%). LC-MS (M+H)⁺=287.10.

### Step 2: Methyl 4-(5-cyano-2-cyclopropylphenyl)-6-methylnicotinate

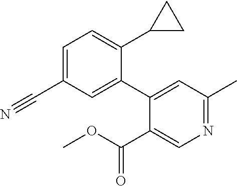 [SMILES: COC(=O)c1cnc(C)cc1-c1cc(C#N)ccc1C1CC1]

To a solution of methyl 4-(2-chloro-5-cyanophenyl)-6-methylnicotinate (500 mg, 1.747 mmol) and cyclopropylboronic acid (165 mg, 1.923 mmol) in dioxane (10 mL) and water (2 mL) was added Pd(OAc)₂ (39 mg, 0.175 mmol), tricyclohexylphosphine (98 mg, 0.35 mmol) and K₃PO₄ (742 mg, 3.494 mmol). The mixture was stirred at 100° C. for 4 h under nitrogen. The reaction mixture was cooled to room temperature and diluted with ethyl acetate (20 mL), washed with brine (20 mL), dried with over sodium sulfate, and concentrated under reduced pressure. The residue was purified by silica gel column chromatography, eluting with methanol in dichloromethane (5%, v/v) to afford title compound (300 mg, 58%).

### Step 3: 4-(5-cyano-2-cyclopropylphenyl)-6-methylnicotinic acid

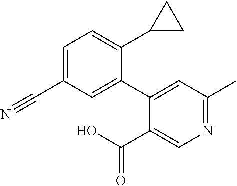 [SMILES: Cc1cc(-c2cc(C#N)ccc2C2CC2)c(C(=O)O)cn1]

The title compound (200 mg, 70%) was prepared in a manner similar to that in Example 1 step 3 from methyl 4-(5-cyano-2-cyclopropylphenyl)-6-methylnicotinate. LC-MS (M+H)⁺=279.1.

### Step 4: N-(5-bromothiazolo[5,4-b]pyridin-2-yl)-4-(5-cyano-2-cyclopropylphenyl)-6-methylnicotinamide

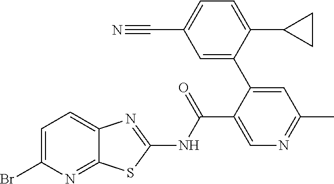 [SMILES: Cc1cc(-c2cc(C#N)ccc2C2CC2)c(C(=O)Nc2nc3ccc(Br)nc3s2)cn1]

The title compound (180 mg, 51%) was prepared in a manner similar to that in Example 11 step 3 from 4-(5-cyano-2-cyclopropylphenyl)-6-methylnicotinic acid and 5-bromothiazolo[5,4-b]pyridin-2-amine. LC-MS (M+H)⁺=490.03.

### Step 5: 4-(5-cyano-2-cyclopropylphenyl)-N-(5-(4-cyanophenyl)thiazolo[5,4-b]pyridin-2-yl)-6-methylnicotinamide

The title compound (40 mg, 21%) was prepared in a manner similar to that in Example 78 step 3 from N-(5-bromothiazolo[5,4-b]pyridin-2-yl)-4-(5-cyano-2-cyclopropylphenyl)-6-methylnicotinamide and (4-cyanophenyl)boronic acid. 1H NMR (400 MHz, d6-DMSO) δ 13.14 (s, 1H), 8.93 (s, 1H), 8.34 (d, J=8.5 Hz, 2H), 8.28-8.18 (m, J=8.6 Hz, 2H), 7.98 (d, J=8.5 Hz, 2H), 7.76 (d, J=8.2 Hz, 1H), 7.71 (s, 1H), 7.43 (s, 1H), 7.03 (d, J=8.2 Hz, 1H), 2.63 (s, 3H), 1.63-1.48 (m, J=12.7, 6.4 Hz, 1H), 0.94-0.77 (m, 3H), 0.56 (s, 1H). LC-MS (M+H)⁺=513.3.

### Example 168: 4-(5-cyano-2-methoxyphenyl)-N-(5-(5-(cyanomethyl)pyridin-2-yl)thiazolo[5,4-b]pyridin-2-yl)-6-methylnicotinamide

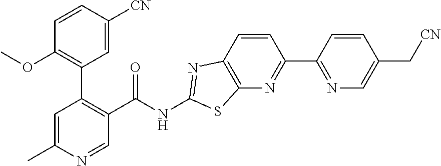 [SMILES: COc1ccc(C#N)cc1-c1cc(C)ncc1C(=O)Nc1nc2ccc(-c3ccc(CC#N)cn3)nc2s1]

### Step 1: 2-(6-(tributylstannyl)pyridin-3-yl)acetonitrile

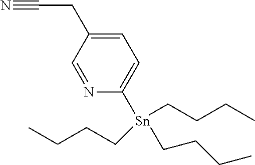 [SMILES: CCC[CH2][Sn]([CH2]CCC)([CH2]CCC)[c]1ccc(CC#N)cn1]

To a mixture of 2-(6-chloropyridin-3-yl)acetonitrile (485 mg, 3.175 mmol), hexabutyldistannane (2765 mg, 4.765 mmol) and LiCl (822 mg, 19.385 mmol) in dioxane (10 mL) were added Pd₂(dba)₃ (150 mg, 0.164 mmol) and tricyclohexylphosphane (85 mg, 0.305 mmol) in portions at room temperature under nitrogen atmosphere. The resulting mixture was stirred for 16 h at 100° C. under nitrogen atmosphere. When the starting material was consumed, the resulting mixture was filtered and the filter cake was washed with ethyl acetate (2×10 mL). The filtrate was quenched with saturated aqueous KF at room temperature. The aqueous phase was extracted with ethyl acetate (3×30 mL). The organic phases were combined, washed with brine, dried over Na₂SO₄ and filtered. The filtrate was concentrated under reduced pressure and the residue was purified by flash chromatography eluting with ethyl acetate in petroleum ether (0% to 30% gradient, v/v) to yield the title compound (440 mg, 34%). LC-MS (M+H)⁺=409.2.

### Step 2: 4-(5-cyano-2-methoxyphenyl)-N-(5-(5-(cyanomethyl)pyridin-2-yl)thiazolo[5,4-b]pyridin-2-yl)-6-methylnicotinamide

To a stirred solution of N-(5-bromothiazolo[5,4-b]pyridin-2-yl)-4-(5-cyano-2-methoxyphenyl)-6-methylnicotinamide (100 mg, 0.208 mmol) and 2-(6-(tributylstannyl)pyridin-3-yl)acetonitrile (150 mg, 0.368 mmol) in N,N-dimethylformamide (4 mL) was added XPhos Pd G3 (19 mg, 0.022 mmol) and XPhos (14 mg, 0.030 mmol,) in portions at room temperature. The resulting mixture was stirred for 16 h at 100° C. under nitrogen atmosphere. After consumption of starting material, the resulting mixture was concentrated under reduced pressure and the residue was purified by flash chromatography eluting with methanol in dichloromethane (0% to 10% gradient). The crude product was purified by prep-HPLC under the following conditions: column, XBridge Shield RP18 OBD Column, 30×150 mm, 5 um; mobile phase, acetonitrile in water (with 10 mmol/L NH₄HCO₃ and 0.1% NH₄OH), 18% to 48% gradient in 10 min; detector, UV 254 nm. The title compound (16 mg, 14%) was obtained. ¹H NMR (400 MHz, d6-DMSO) δ 13.09 (brs, 1H), 8.81 (s, 1H), 8.70-8.65 (m, 1H), 8.54-8.42 (m, 2H), 8.28-8.21 (m, 1H), 8.00-7.89 (m, 3H), 7.45 (s, 1H), 7.22-7.15 (m, 1H), 4.20 (s, 2H), 3.60 (s, 3H), 2.61 (s, 3H). LC-MS (M+H)⁺=518.1.

### Example 169: 4-(5-cyano-2-methoxyphenyl)-N-(5-(4-cyano-2-methylphenyl)thiazolo[5,4-b]pyridin-2-yl)-6-methylnicotinamide

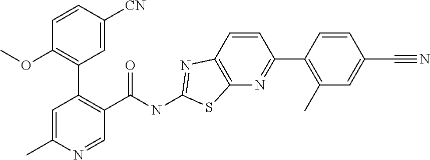 [SMILES: COc1ccc(C#N)cc1-c1cc(C)ncc1C(=O)Nc1nc2ccc(-c3ccc(C#N)cc3C)nc2s1]

The title compound (35 mg, 47%) was prepared in a manner similar to that in Example 78 step 3 from N-(5-bromothiazolo[5,4-b]pyridin-2-yl)-4-(5-cyano-2-methoxyphenyl)-6-methylnicotinamide and (4-cyano-2-methylphenyl)boronic acid. ¹H NMR (400 MHz, d6-DMSO) δ:13.02 (s, 1H), 8.76 (s, 1H), 8.19 (d, J=8.4 Hz, 1H), 7.88 (d, J=6.4 Hz, 2H), 7.81 (s, 1H), 7.74 (d, J=8.1 Hz, 1H), 7.67 (d, J=8.4 Hz, 1H), 7.62 (d, J=8.0 Hz, 1H), 7.40 (s, 1H), 7.14 (d, J=9.2 Hz, 1H), 3.56 (s, 3H), 2.56 (s, 3H), 2.37 (s, 3H). LC-MS: (M+H)⁺=517.6.

### Example 170: N-(5-(4-(azetidin-3-yloxy)phenyl)thiazolo[5,4-b]pyridin-2-yl)-4-(5-cyano-2-methoxyphenyl)-6-methylnicotinamide

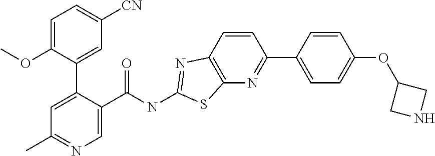 [SMILES: COc1ccc(C#N)cc1-c1cc(C)ncc1C(=O)Nc1nc2ccc(-c3ccc(OC4CNC4)cc3)nc2s1]

### Step 1: tert-butyl 3-(4-(5,5-dimethyl-1,3,2-dioxaborinan-2-yl)phenoxy)azetidine-1-carboxylate

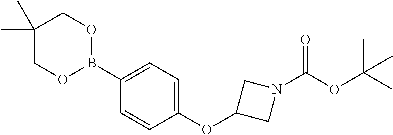 [SMILES: CC1(C)COB(c2ccc(OC3CN(C(=O)OC(C)(C)C)C3)cc2)OC1]

The title compound (330 mg, 91%) was prepared in a manner similar to Example 28 of Step 2 from tert-butyl 3-(4-bromophenoxy)azetidine-1-carboxylate and 5,5,5′,5′-tetramethyl-2,2′-bi(1,3,2-dioxaborinane). LC-MS (M+H)⁺=362.3.

### Step 2: tert-butyl 3-(4-(2-(4-(5-cyano-2-methoxyphenyl)-6-methylnicotinamido)thiazolo[5,4-b]pyridin-5-yl)phenoxy)azetidine-1-carboxylate

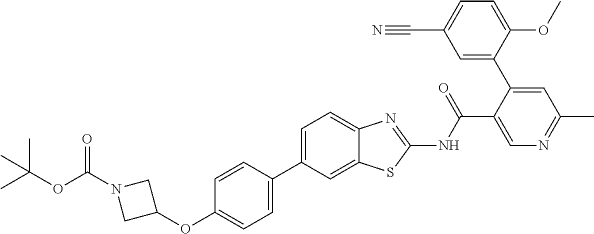 [SMILES: COc1ccc(C#N)cc1-c1cc(C)ncc1C(=O)Nc1nc2ccc(-c3ccc(OC4CN(C(=O)OC(C)(C)C)C4)cc3)cc2s1]

The title compound (74 mg, 41%) was prepared in a manner similar to Example 1 of Step 5 from N-(5-bromothiazolo[5,4-b]pyridin-2-yl)-4-(5-cyano-2-methoxyphenyl)-6-methylnicotinamide and tert-butyl 3-(4-(5,5-dimethyl-1,3,2-dioxaborinan-2-yl)phenoxy)azetidine-1-carboxylate. LC-MS (M+H)⁺=649.3

### Step 3: N-(5-(4-(azetidin-3-yloxy)phenyl]thiazolo[5,4-b]pyridin-2-yl)-4-(5-cyano-2-methoxyphenyl)-6-methylnicotinamide

The title compound (10 mg, 16%) was prepared in a manner similar to Example 1 of Step 6 from tert-butyl 3-(4-(2-(4-(5-cyano-2-methoxyphenyl)-6-methylnicotinamido)thiazolo[5,4-b]pyridin-5-yl)phenoxy)azetidine-1-carboxylate. 1H NMR (400 MHz, d6-DMSO) δ 8.84 (s, 1H), 8.22 (s, 1H), 8.05 (s, 3H), 7.99-7.76 (m, 3H), 7.38 (s, 1H), 7.18 (d, J=8.0 Hz, 1H), 6.93 (d, J=8.0 Hz, 2H), 5.09 (brs, 1H), 4.11 (brs, 2H), 3.76 (brs, 2H), 3.60 (s, 3H), 2.58 (s, 3H). LC-MS (M+H)⁺=549.3.

### Example 171: 4-(5-cyano-2-methoxyphenyl)-N-(5-(4-(cyanomethyl)-3-methylphenyl)thiazolo[5,4-b]pyridin-2-yl)-6-methylnicotinamide

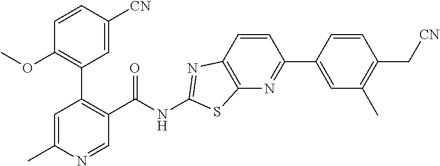 [SMILES: COc1ccc(C#N)cc1-c1cc(C)ncc1C(=O)Nc1nc2ccc(-c3ccc(CC#N)c(C)c3)nc2s1]

The title compound (23 mg, 9%) was prepared in a manner similar to that described in Example 1 step 5 from N-(5-bromothiazolo[5,4-b]pyridin-2-yl)-4-(5-cyano-2-methoxyphenyl)-6-methylnicotinamide and 2-(2-methyl-4-(4,4,5,5-tetramethyl-1,3,2-dioxaborolan-2-yl)phenyl)acetonitrile. ¹H NMR (400 MHz, d6-DMSO) δ: 13.00 (s, 1H), 8.81 (s, 1H), 8.12-8.06 (m, 1H), 8.05-8.00 (m, 1H), 8.00-7.95 (m, 2H), 7.53-7.40 (m, 2H), 7.47-7.41 (s, 2H), 7.23-7.15 (m, 1H), 4.07 (s, 2H), 3.60 (s, 3H), 2.60 (s, 3H), 2.40 (s, 3H). LC-MS (M+H)⁺=531.3.

### Example 172: 4-(5-cyano-2-methoxyphenyl)-N-(5-(4-(cyanomethyl)-2-methylphenyl)thiazolo[5,4-b]pyridin-2-yl)-6-methylnicotinamide

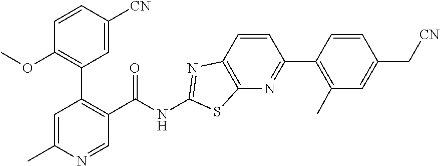 [SMILES: COc1ccc(C#N)cc1-c1cc(C)ncc1C(=O)Nc1nc2ccc(-c3ccc(CC#N)cc3C)nc2s1]

### Step 1: 2-(3-methyl-4-(4,4,5,5-tetramethyl-1,3,2-dioxaborolan-2-yl)phenyl)acetonitrile

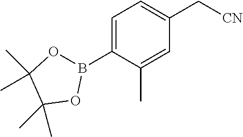 [SMILES: Cc1cc(CC#N)ccc1B1OC(C)(C)C(C)(C)O1]

To a solution of 2-(4-bromo-3-methylphenyl)acetonitrile (1 g, 4.76 mmol) and bis(pinacolato)diboron (3.63 g, 14.3 mmol) in DMSO (27 mL) was added bis(diphenylphosphino)ferrocene dichloropalladium(II) (348 mg, 0.476 mmol) and KOAc (934 mg, 9.52 mmol). The mixture was stirred at 90° C. for 2 h under nitrogen. The reaction mixture was cooled to room temperature and diluted with ethyl acetate (50 mL), washed with water and brine (50 mL), dried with over sodium sulfate, filtered and concentrated under reduced pressure. The residue was purified by silica gel chromatography eluting with ethyl acetate in petroleum ether (10%, v/v) to give the title compound (200 mg, 16%). LC-MS (M+H)⁺=258.2.

### Step 2: 4-(5-cyano-2-methoxyphenyl)-N-(5-(4-(cyanomethyl)-2-methylphenyl)thiazolo[5,4-b]pyridin-2-yl)-6-methylnicotinamide

The title compound (26 mg, 33%) was prepared in a manner similar to that in Example 78 step 3 from N-(5-bromothiazolo[5,4-b]pyridin-2-yl)-4-(5-cyano-2-methoxyphenyl)-6-methylnicotinamide and 2-(3-methyl-4-(4,4,5,5-tetramethyl-1,3,2-dioxaborolan-2-yl)phenyl)acetonitrile. ¹H NMR (400 MHz, d6-DMSO) δ:12.99 (s, 1H), 8.81 (s, 1H), 8.17 (t, J=9.0 Hz, 1H), 7.91 (d, J=7.9 Hz, 2H), 7.63 (d, J=8.3 Hz, 1H), 7.49 (d, J=7.7 Hz, 1H), 7.43 (s, 1H), 7.29 (d, J=9.7 Hz, 2H), 7.18 (d, J=8.7 Hz, 1H), 4.08 (s, 2H), 3.60 (s, 3H), 2.60 (s, 3H), 2.37 (s, 3H). LC-MS (M+H)⁺=531.3.

### Example 173: 5-cyano-N-(5-(4-cyanophenyl)thiazolo[5,4-b]pyridin-2-yl)-1,2,6′-trimethyl-6-oxo-1,6-dihydro-[3,4′-bipyridine]-3′-carboxamide

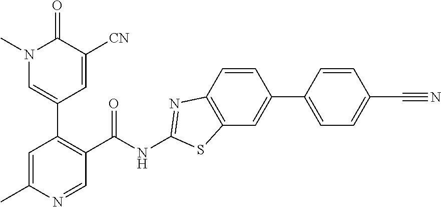 [SMILES: Cc1cc(-c2cc(C#N)c(=O)n(C)c2)c(C(=O)Nc2nc3ccc(-c4ccc(C#N)cc4)cc3s2)cn1]

### Step 1: 5-bromo-1,6-dimethyl-2-oxo-1,2-dihydroyridine-3-carbonitrile

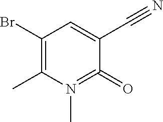

The title compound (601 mg, 61%) was prepared in a manner similar to that described in Example 87 step 1 from 5-bromo-6-methyl-2-oxo-1,2-dihydropyridine-3-carbonitrile and iodomethane. LC-MS (M+H)⁺=226.9.

### Step 2: 1,6-dimethyl-2-oxo-5-(4,4,5,5-tetramethyl-1,3,2-dioxaborolan-2-yl)-1,2-dihydropyridine-3-carbonitrile

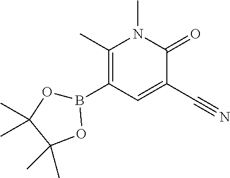 [SMILES: Cc1c(B2OC(C)(C)C(C)(C)O2)cc(C#N)c(=O)n1C]

The title compound (380 mg, 85%) was prepared in a manner similar to that described in Example 28 step 2 from 5-bromo-1,6-dimethyl-2-oxo-1,2-dihydropyridine-3-carbonitrile. LC-MS (M+H)⁺=275.1.

### Step 3: methyl 5-cyano-1,2,6′-trimethyl-6-oxo-1,6-dihydro-[3,4′-bipyridine]-3′-carboxylate

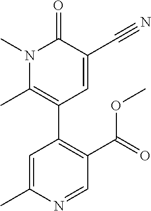 [SMILES: COC(=O)c1cnc(C)cc1-c1cc(C#N)c(=O)n(C)c1C]

The title compound (280 mg, 81%) was prepared in a manner similar to that described in Example 2 step 2 from 1,6-dimethyl-2-oxo-5-(4,4,5,5-tetramethyl-1,3,2-dioxaborolan-2-yl)-1,2-dihydropyridine-3-carbonitrile and methyl 4-chloro-6-methylnicotinate. LC-MS (M+H)⁺=298.1

### Step 4: 5-cyano-1,2,6′-trimethyl-6-oxo-1,6-dihydro-[3,4′-bipyridine]-3′-carboxylic acid

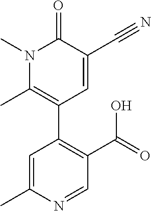 [SMILES: Cc1cc(-c2cc(C#N)c(=O)n(C)c2C)c(C(=O)O)cn1]

The title compound (140 mg, 89%) was prepared in a manner similar to that described in Example 1 step 3 from methyl 5-cyano-1,2,6′-trimethyl-6-oxo-1,6-dihydro-[3,4′-bipyridine]-3′-carboxylate. LC-MS (M+H)⁺=284.0.

### Step 5: 5-cyano-N-(5-(4-cyanophenyl)thiazolo[5,4-b]pyridin-2-yl)-1,2,6′-trimethyl-6-oxo-1,6-dihydro-[3,4′-bipyridine]-3′-carboxamide

The title compound (8.5 mg, 4.5%) was prepared in a manner similar to that described in Example 11 step 3 from 5-cyano-1,2,6′-trimethyl-6-oxo-1,6-dihydro-[3,4′-bipyridine]-3′-carboxylic acid and 4-(2-aminothiazolo[5,4-b]pyridin-5-yl)benzonitrile. ¹H NMR (400 MHz, d6-DMSO) δ: 13.17 (s, 1H), 9.02 (s, 1H), 8.40-8.28 (m, 2H), 8.14 (m, 2H), 8.02 (m, 1H), 8.01-8.90 (m, 2H), 7.28 (s, 1H), 3.55 (s, 3H), 2.58 (s, 3H), 2.23 (s, 3H). LC-MS (M+H)⁺=518.3.

### Example 174: 4-(5-cyano-2-methoxyphenyl)-6-methyl-N-(5-(2-methyl-4-((2-oxopyrrolidin-1-yl)methyl)phenyl)thiazolo[5,4-b]p ridin-2-yl)nicotinamide

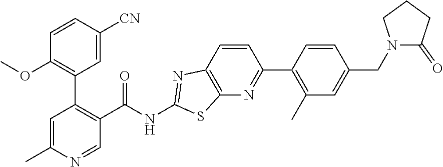 [SMILES: COc1ccc(C#N)cc1-c1cc(C)ncc1C(=O)Nc1nc2ccc(-c3ccc(CN4CCCC4=O)cc3C)nc2s1]

### Step 1: 1-(4-bromo-3-methylbenzyl)pyrrolidin-2-one

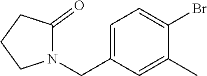

To a solution of pyrrolidin-2-one (161 mg, 1.894 mmol) in N,N-dimethylformamide (7.5 mL) was added NaH (60%, 83 mg, 1.894 mmol) at 0° C. The mixture was stirred at 0° C. for 20 min under nitrogen and then 1-bromo-4-(bromomethyl)-2-methylbenzene (500 mg, 1.894 mmol) was added. The resulting mixture was stirred at 25° C. for 18 h under nitrogen. Water was added, extracted with ethyl acetate (20 mL), washed with brine (20 mL), dried with over sodium sulfate, and concentrated under reduced pressure. The residue was purified by silica gel column chromatography eluting with ethyl acetate in petroleum ether (33%, v/v) to give compound 26-1 (307 mg, 60%). LC-MS (M+H)⁺=268.0.

### Step 2: 1-(3-methyl-4-(4,4,5,5-tetramethyl-1,3,2-dioxaborolan-2-yl)benzyl)pyrrolidin-2-one

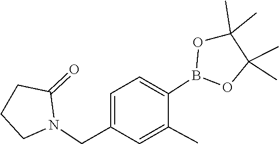 [SMILES: Cc1cc(CN2CCCC2=O)ccc1B1OC(C)(C)C(C)(C)O1]

The title compound (65 mg, 18%) was prepared in a manner similar to that in Example 28 step 2 from 1-(4-bromo-3-methylbenzyl)pyrrolidin-2-one and bis(pinacolato)diboron. LC-MS (M+H)⁺=316.2.

### Step 3: 4-(5-cyano-2-methoxyphenyl)-6-methyl-N-(5-(2-methyl-4-((2-oxopyrrolidin-1-yl)methyl)phenyl)thiazolo[5,4-b]pyridin-2-yl)nicotinamide

The title compound (12 mg, 14%) was prepared in a manner similar to that in Example 78 step 3 from N-(5-bromothiazolo[5,4-b]pyridin-2-yl)-4-(5-cyano-2-methoxyphenyl)-6-methylnicotinamide and 1-(3-methyl-4-(4,4,5,5-tetramethyl-1,3,2-dioxaborolan-2-yl)benzyl)pyrrolidin-2-one. ¹H NMR (400 MHz, d6-DMSO) δ:12.99 (s, 1H), 8.80 (s, 1H), 8.17 (d, J=8.4 Hz, 1H), 7.92 (d, J=7.5 Hz, 2H), 7.62 (d, J=8.4 Hz, 1H), 7.43 (d, J=7.4 Hz, 2H), 7.17 (dd, J=13.3, 8.7 Hz, 3H), 4.40 (s, 2H), 3.60 (s, 3H), 3.27 (t, J=7.0 Hz, 2H), 2.60 (s, 3H), 2.35 (s, 3H), 2.32 (t, J=8.1 Hz, 2H), 2.01-1.89 (m, 2H). LC-MS (M+H)⁺=589.6.

### Example 175: N-(5-(4-carbamoyl-3-methylphenyl)thiazolo[5,4-b]pyridin-2-yl)-4-(2-methoxy-5-(methylsulfonyl)phenyl)-6-methylnicotinamide

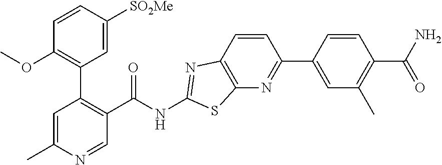 [SMILES: COc1ccc(S(C)(=O)=O)cc1-c1cc(C)ncc1C(=O)Nc1nc2ccc(-c3ccc(C(N)=O)c(C)c3)nc2s1]

### Step 1: methyl 4-(2-methoxy-5-(methylsulfonyl)phenyl)-6-methylnicotinate

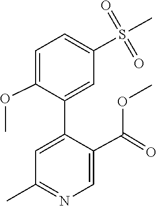 [SMILES: COC(=O)c1cnc(C)cc1-c1cc(S(C)(=O)=O)ccc1OC]

The title compound (535 mg, 83%) was prepared in a manner similar to that in Example 2 step 2 from methyl 4-chloro-6-methylnicotinate and 2-(2-methoxy-5-(methylsulfonyl)phenyl)-4,4,5,5-tetramethyl-1,3,2-dioxaborolane. LC-MS (M+H)⁺=336.0.

### Step 2: 4-(2-methoxy-5-(methylsulfonyl)phenyl)-6-methylnicotinic acid

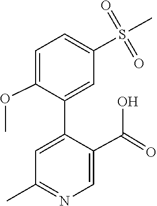 [SMILES: COc1ccc(S(C)(=O)=O)cc1-c1cc(C)ncc1C(=O)O]

The title compound (450 mg, 93%) was prepared in a manner similar to that in Example 1 step 3 from methyl 4-(2-methoxy-5-(methylsulfonyl)phenyl)-6-methylnicotinate. LC-MS (M+H)⁺=322.0.

### Step 3: N-(5-bromothiazolo[5,4-b]pyridin-2-yl)-4-(2-methoxy-5-(methylsulfonyl)phenyl)-6-methylnicotinamide

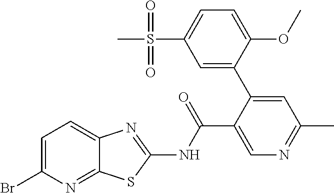 [SMILES: COc1ccc(S(C)(=O)=O)cc1-c1cc(C)ncc1C(=O)Nc1nc2ccc(Br)nc2s1]

The title compound (90 mg, 36%) was prepared in a manner similar to that in Example 11 step 3 from 5-bromothiazolo[5,4-b]pyridin-2-amine and 4-(2-methoxy-5-(methylsulfonyl)phenyl)-6-methylnicotinic acid. LC-MS (M+H)⁺=533.1.

### Step 4: N-(5-(4-carbamoyl-3-methylphenyl)thiazolo[5,4-b]pyridin-2-yl)-4-(2-methoxy-5-(methylsulfonyl)phenyl)-6-methylnicotinamide

The title compound (16 mg, 18%) was prepared in a manner similar to that in Example 78 step 3 from 2-methyl-4-(4,4,5,5-tetramethyl-1,3,2-dioxaborolan-2-yl)benzamide and N-(5-bromothiazolo[5,4-b]pyridin-2-yl)-4-(2-methoxy-5-(methylsulfonyl)phenyl)-6-methylnicotinamide. ¹H NMR (300 MHz, d6-DMSO) δ 13.01 (brs, 1H), 8.83 (s, 1H), 8.16-7.88 (m, 6H), 7.78 (s, 1H), 7.52-7.37 (m, 3H), 7.22 (d, J=8.8 Hz, 1H), 3.61 (s, 3H), 3.25 (s, 3H), 2.60 (s, 3H), 2.46 (s, 3H). LC-MS (M+H)⁺=588.1.

### Example 176: 4-(5-cyano-2-methoxyphenyl)-N-(5-(4-cyano-2-methylphenyl)thiazolo[5,4-b]pyridin-2-yl)-6-(hydroxymethyl)nicotinamide

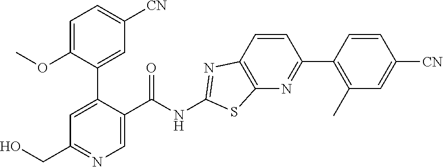 [SMILES: COc1ccc(C#N)cc1-c1cc(CO)ncc1C(=O)Nc1nc2ccc(-c3ccc(C#N)cc3C)nc2s1]

### Step 1: (5-((5-bromothiazolo[5,4-b]pyridin-2-yl)carbamoyl)-4-(5-cyano-2-methoxyphenyl)pyridin-2-yl)methyl acetate

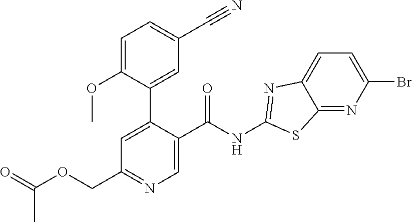 [SMILES: COc1ccc(C#N)cc1-c1cc(COC(C)=O)ncc1C(=O)Nc1nc2ccc(Br)nc2s1]

The title compound (75 mg, 42%) was prepared in a manner similar to that in Example 11 step 3 from 6-(acetoxymethyl)-4-(5-cyano-2-methoxyphenyl)nicotinic acid and 5-bromothiazolo[5,4-b]pyridin-2-amine. LC-MS (M+H)⁺=540.1.

### Step 2: (4-(5-cyano-2-methoxyphenyl)-5-((5-(4-cyano-2-methylphenyl)thiazolo[5,4-b]pyridin-2-yl)carbamoyl)pyridin-2-yl)methyl acetate

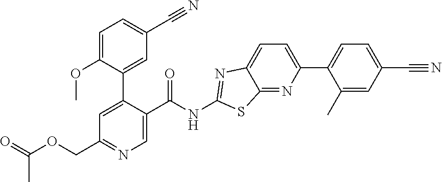 [SMILES: COc1ccc(C#N)cc1-c1cc(COC(C)=O)ncc1C(=O)Nc1nc2ccc(-c3ccc(C#N)cc3C)nc2s1]

The title compound (50 mg, 58%) was prepared in a manner similar to that in Example 1 step 5 from 3-methyl-4-(4,4,5,5-tetramethyl-1,3,2-dioxaborolan-2-yl)benzonitrile and (5-((5-bromothiazolo[5,4-b]pyridin-2-yl)carbamoyl)-4-(5-cyano-2-methoxyphenyl)pyridin-2-yl)methyl acetate. LC-MS (M+H)⁺=575.1

### Step 3: 4-(5-cyano-2-methoxyphenyl)-N-(5-(4-cyano-2-methylphenyl)thiazolo[5,4-b]pyridin-2-yl)-6-(hydroxymethyl)nicotinamide

The title compound (6.5 mg, 13%) was prepared in a manner similar to that described in Example 1 step 3 from (4-(5-cyano-2-methoxyphenyl)-5-((5-(4-cyano-2-methylphenyl)thiazolo[5,4-b]pyridin-2-yl)carbamoyl)pyridin-2-yl)methyl acetate. ¹H NMR (400 MHz, d6-DMSO) δ: 13.09 (s, 1H), 8.87 (s, 1H), 8.22-8.17 (m, 1H), 8.00-7.74 (m, 4H), 7.75-7.60 (m, 2H), 7.54-7.39 (m, 1H), 7.25-7.15 (m, 1H), 5.68-5.58 (m, 1H), 4.74-4.65 (m, 2H), 3.61 (s, 3H), 2.41 (s, 3H). LC-MS (M+H)⁺=533.2.

### Example 177A/Example 177B: (R)-4-(5-cyano-2-methoxyphenyl)-N-(5-(4-(1-cyanoethyl)phenyl)thiazolo[5,4-b]pyridin-2-yl)-6-methylnicotinamide & (S)-4-(5-cyano-2-methoxyphenyl)-N-(5-(4-(1-cyanoethyl)phenyl)thiazolo[5,4-b]pyridin-2-yl)-6-methylnicotinamide

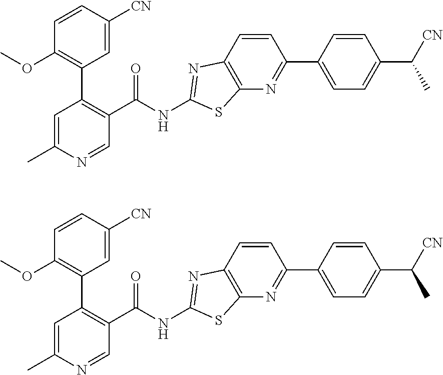 [SMILES: COc1ccc(C#N)cc1-c1cc(C)ncc1C(=O)Nc1nc2ccc(-c3ccc([C@@H](C)C#N)cc3)nc2s1 ; COc1ccc(C#N)cc1-c1cc(C)ncc1C(=O)Nc1nc2ccc(-c3ccc([C@H](C)C#N)cc3)nc2s1]

### Step 1: 2-(4-(4,4,5,5-tetramethyl-1,3,2-dioxaborolan-2-yl)phenyl)propanenitrile

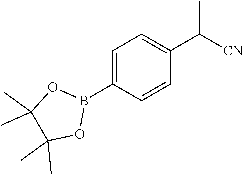 [SMILES: CC(C#N)c1ccc(B2OC(C)(C)C(C)(C)O2)cc1]

The title compound (540 mg, 70%) was prepared in a manner similar to that described in Example 28 step 2 from 2-(4-bromophenyl)propanenitrile and bis(pinacolato)diboron. LC-MS (M+H)⁺=258.1.

### Step 2: (R)-4-(5-cyano-2-methoxyphenyl)-N-(5-(4-(1-cyanoethyl)phenyl)thiazolo[5,4-b]pyridin-2-yl)-6-methylnicotinamide & (S)-4-(5-cyano-2-methoxyphenyl)-N-(5-(4-(1-cyanoethyl)phenyl)thiazolo[5,4-b]pyridin-2-yl)-6-methylnicotinamide

Example 177A/177B was prepared in a manner similar to that described in Example 10 step 2 from N-(5-bromothiazolo[5,4-b]pyridin-2-yl)-4-(5-cyano-2-methoxyphenyl)-6-methylnicotinamide and 2-(4-(4,4,5,5-tetramethyl-1,3,2-dioxaborolan-2-yl)phenyl)propanenitrile. The two isomeric products were obtained by separation on chiral-HPLC under the following conditions: Column, CHIRALPAK IH-3, 0.46×50 cm, 3 um. Mobile phase: isopropanol:dichloromethane=1:1 (0.1% DEA), 30% isocratic in 3 min; detector, 220 nm.

Example 177A (enantiomer 1, 14 mg, 7%) ¹H NMR (400 MHz, d6-DMSO) δ: 13.02 (s, 1H), 8.80 (s, 1H), 8.21-8.11 (m, 3H), 8.10-8.02 (m, 1H), 7.98-7.85 (m, 2H), 7.61-7.50 (m, 2H), 7.47-7.41 (s, 1H), 7.23-7.15 (m, 1H), 4.46-4.35 (m, 1H), 3.60 (s, 3H), 2.60 (s, 3H), 1.62-1.55 (d, 3H). LC-MS (M+H)⁺=531.2. Chiral-HPLC RT=1.710 min.

Example 177B (enantiomer 2, 14 mg, 7%)¹H NMR (400 MHz, d6-DMSO) δ:13.02 (s, 1H), 8.80 (s, 1H), 8.21-8.11 (m, 3H), 8.10-8.02 (m, 1H), 7.98-7.85 (m, 2H), 7.61-7.50 (m, 2H), 7.47-7.41 (s, 1H), 7.23-7.15 (m, 1H), 4.46-4.35 (m, 1H), 3.60 (s, 3H), 2.60 (s, 3H), 1.62-1.55 (d, 3H). LC-MS (M+H)⁺=531.2. Chiral-HPLC RT=2.180 min.

### Biological Assays

Baculovirus Expression of Human POL theta (1-894)

Human POL Theta (1-894)-His-pFastBac1 baculovirus samples were generated using the Bac-to-Bac baculovirus expression system (Invitrogen) according to the manufacturer's protocol. The DNA used for expression of POL Theta (1-894)-His have SEQ ID (NCBI Reference Sequence: NM_199420.4). Baculovirus amplification was achieved using infected SF9 cells at 1:2000 virus/cells ratios, and grown for 96 hours at 27° C. post-transfection. The expression scaled up for each protein was carried out in the flask 3 L from CORNING. 4 L of 3×106 cells/mL Sf9 cells (Expression System, Invitrogen) grown in SF900TM II SFM insect medium (Expression System) were infected with virus stock at 1:200 virus/cell ratios, and grown for 48 hours at 27° C. post-transfection. The infected cell culture was harvested by centrifugation at 6000 rpm for 15 min 4° C. in a SORVALL LYNX6000 centrifuge. The cell pellets were stored at −80° C.

### Purification of Human POL (1-894)-Theta-his

The human POL Theta (1-894)-His produced as described above, were purified from Sf9 baculovirus-infected insect cell paste. The cells were lysed using sonication method, and the lysates were clarified by centrifugation. The clarified lysates were purified to ˜90% homogeneity, using two successive column chromatography steps on an AKTA Purifier system. The two steps column chromatography included nickel affinity resin capture (i.e. Ni-NTA Agarose, Qiagen), followed by size exclusion chromatography (i.e. Hiload16/60 Superdex200 prep grade, GE Healthcare. The proteins were delivered and stored at −80° C. The formulation buffers were identical for three proteins: 25 mM Tris, 150 mmol/L NaCl, 2 mM DTT, pH8.0.

### Biochemical Pol Theta (1-894) ATPase IC50 Assay

Enzymatic reactions of Pol Theta (1-894) were performed using ADP-Glo assay with ssDNA substrate. Pol Theta (1-894) was expressed in baculovirus expression system. The inhibition activities testing for the compound disclosed herein were carried out at room temperature in assay buffer containing 40 mM Tris pH 7.5, 20 mM MgCl2, 1 mM DTT, 0.01% BSA and 0.05% Tween-20. Compounds in DMSO were dispensed into wells of a black 384 well plate (Corning 4514) using D300e digital dispenser (Tecan). The ranges of compounds final concentration were 2.62 nM˜10000 nM. 3 μL 2× enzyme solution containing 12.5 nM enzyme was added to wells. After incubation for 1 hour, 3 μL 2× substates solution containing 0.124 μM ssDNA (5′-ACT CGT CTC TAG CTT TTT-3′) and 444 μM ATP was added to the wells to initiate reaction. After 1 hour reaction, 5 μL ADP-Glo reagent (Promga V9102) was added and incubated for 40 minutes. 10 μL Kinase Detection reagent was added and incubated for 40 minutes. Luminescence was measured on a microplate reader (PHERAstar FSX, BMG labtech). The IC50 values were calculated based on inhibition of enzyme activity in the presence of increasing concentrations of compounds. IC50 values of the compounds disclosed herein for Pol Theta (1-894) are shown in Table.

TABLE 1
Enzymatic activity IC50 (nM)
for the compounds disclosed herein

| | Biochemical |
|---|---|
| Example No. | IC50 (nM) |
| 1 | 347 |
| 2 | 191 |
| 3 | 145 |
| 4 | 1562 |
| 5 | 186 |
| 6 | 359 |
| 7 | 92 |
| 8 | 12 |
| 9 | 20 |
| 10 | 149 |
| 11 | 57 |
| 12 | 324 |
| 13 | 147 |
| 14 | 143 |
| 15 | 206 |
| 16 | 8.2 |
| 17 | 8.1 |
| 18 | 11 |
| 19 | 8.5 |
| 20 | 54 |
| 21 | 81 |
| 22 | 33 |
| 23 | 22 |
| 26 | 189 |
| 25 | 54 |
| 24 | 62 |
| 27 | 67 |
| 28 | 256 |
| 29 | 57 |
| 30 | 222 |
| 31 | 70 |
| 32 | 26 |
| 33 | 8.5 |
| 34 | 16 |
| 35 | 94 |
| 36 | 73 |
| 37 | 151 |
| 38 | 11 |
| 39 | 73 |
| 40 | 87 |
| 41 | 91 |
| 42 | 65 |
| 43 | 75 |
| 44 | 14 |
| 45 | 32 |
| 46 | 129 |
| 47 | 99 |
| 48 | 9.5 |
| 49 | 150 |
| 50 | 218 |
| 51 | 15 |
| 52 | 8.4 |
| 53 | 78 |
| 54 | 64 |
| 55 | 5 |
| 56 | 15 |
| 57 | 58 |
| 58 | 101 |
| 59 | 98 |
| 60 | 361 |
| 61 | 121 |
| 62 | 24 |
| 63 | 31 |
| 64 | 186 |
| 65 | 224 |
| 66 | 99 |
| 67 | 64 |
| 68 | 253 |
| 69 | 45 |
| 70 | 193 |
| 71 | 83 |
| 72 | 59 |
| 74 | 73 |
| 73 | 15 |
| 75 | 32 |
| 76 | 5.8 |
| 77 | 50 |
| 78 | 7.5 |
| 79 | 41 |
| 80 | 26 |
| 81 | 7.7 |
| 82 | 272 |
| 83 | 6.2 |
| 84 | 49 |
| 85 | 62 |
| 86 | 7.4 |
| 87 | 371 |
| 88 | 50 |
| 89 | 23 |
| 90 | 4.1 |
| 91 | 117 |
| 92 | 16 |
| 93 | 20 |
| 94 | 29 |
| 95 | 72 |
| 96 | 60 |
| 97 | 27 |
| 98 | 22 |
| 99 | 66 |
| 100 | 29 |
| 101 | 35 |
| 102 | 63 |
| 103 | 83 |
| 104 | 6.5 |
| 105 | 16 |
| 106 | 26 |
| 107 | 17 |
| 108 | 19 |
| 109 | 4.7 |
| 110 | 5.8 |
| 111 | 11 |
| 112 | 60 |
| 113 | 167 |
| 114 | 99 |
| 115 | 7.1 |
| 116 | 5.6 |
| 117 | 12 |
| 118 | 17 |
| 119 | 5.2 |
| 120 | 7.9 |
| 121 | 4.9 |
| 122 | 14 |
| 123 | 27 |
| 124 | 44 |
| 125 | 25 |
| 126 | 12 |
| 127 | 35 |
| 128 | 8.1 |
| 129 | 23 |
| 130 | 35 |
| 131 | 14 |
| 132 | 12 |
| 133 | 11 |
| 134 | 15 |
| 135 | 23 |
| 136 | 41 |
| 137 | 54 |
| 138 | 263 |
| 139 | 21 |
| 140 | 7.7 |
| 141 | 3.1 |
| 142 | 5.4 |
| 143 | 22 |
| 144 | 11 |
| 145 | 22 |
| 146 | 22 |
| 147 | 53 |
| 148 | 8.4 |
| 149 | 18 |
| 150 | 43 |
| 151 | 73 |
| 152 | 21 |
| 153 | 57 |
| 154 | 60 |
| 155 | 73 |
| 156 | 14 |
| 157 | 70 |
| 158 | 8.2 |
| 159 | 5 |
| 160 | 63 |
| 161 | 36 |
| 162 | 29 |
| 163 | 28 |
| 164 | 37 |
| 165 | 8.9 |
| 166 | 13 |
| 167 | 15 |
| 168 | 4.4 |
| 169 | 7.5 |
| 170 | 26 |
| 171 | 13 |
| 172 | 10 |
| 173 | 26 |
| 174 | 11 |
| 175 | 8.4 |
| 176 | 9.1 |
| 177A | 25 |
| 177B | 13 |

1. The compound of formula (I): 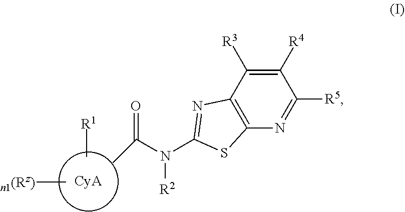
or a N-oxide thereof, or a pharmaceutically acceptable salt thereof, or a stereoisomer thereof, or a tautomer thereof, or a deuterated analog thereof, or a prodrug thereof,
wherein:
ring CyA is a 5- to 12-membered heteroaromatic ring comprising 1 to 4 heteroatoms selected from N, O, or S as ring member(s);
R¹ is —C₁₋₈alkyl, —C₂₋₈alkenyl, —C₂₋₈alkynyl, cycloalkyl, haloalkyl, heterocyclyl, aryl, heteroaryl, oxo, —CN, —OR^{1a}, —SO₂R^{1a}, —SO₂NR^{1a}R^{1b}, —P(O)R^{1a}R^{1b}, —COR^{1a}, —CO₂R^{1a}, —CONR^{1a}R^{1b}, —NR^{1a}R^{1b}, —NR^{1a}COR^{1b}, —NR^{1a}CO₂R^{1b}, —NR^{1a}CONR^{1b}R^{1c}, or —NR^{1a}SO₂R^{1b}; wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{1d};
R^{1a}, R^{1b} and R^{1c} are each independently selected from hydrogen, —C₁₋₈alkyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{1f}; or
(R^{1a} and R^{1b}), (R^{1b} and R^{1c}) or (R^{1a} and R^{1c}), together with the atom(s) to which they are attached, form a 3- to 12-membered ring, said ring comprising 0, 1 or 2 heteroatom(s) independently selected from nitrogen, oxygen, or optionally oxidized sulfur as ring member(s), said ring is optionally substituted with at least one substituent R^{1f};
R^{1d} and R^{1f} are each independently selected from hydrogen, halogen, —C₁₋₈alkyl, —C₂₋₈alkenyl, —C₂₋₈alkynyl, cycloalkyl, heterocyclyl, aryl, heteroaryl, oxo, —CN, —OR^{1g}, —SO₂R^{1g}, —SO₂NR^{1g}R^{1h}, —P(O)R^{1g}R^{1h}, —COR^{1g}, —CO₂R^{1g}, —CONR^{1g}R^{1h}, —NO₂, —NR^{1g}R^{1h}, —NR^{1g}COR^{1h}, —NR^{1g}CO₂R^{1h}, —NR^{1g}CONR^{1h}R¹ⁱ, or —NR^{1g}SO₂R^{1h}; wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{1j}; or
two R^{1d} or two R^{1f}, together with the atom(s) to which they are attached, form a 3- to 12-membered ring, said ring comprising 0, 1 or 2 heteroatom(s) independently selected from nitrogen, oxygen, or optionally oxidized sulfur as ring member(s), said ring is optionally substituted with at least one substituent R^{1k};
R^{1g}, R^{1h} and R¹ⁱ are each independently selected from hydrogen, —C₁₋₈alkyl, —C₂₋₈alkenyl, —C₂₋₈ alkynyl, cycloalkyl, heterocyclyl, aryl or heteroaryl, wherein each of said —C₁₋₈alkyl, —C₂₋₈alkenyl, —C₂₋₈ alkynyl, cycloalkyl, heterocyclyl, aryl or heteroaryl is optionally substituted with at least one substituent halogen, hydroxy, —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R^{1j} and R^{1k} are each independently selected from hydrogen, halogen, —C₁₋₈alkyl, —C₂₋₈alkenyl, —C₂₋₈ alkynyl, cycloalkyl, heterocyclyl, aryl, heteroaryl, oxo, —CN, —OR^{1l}, —SO₂R^{1l}, —SO₂NR^{1l}R^{1m}, —COR^{1l}, —CO₂R^{1l}, —CONR^{1l}R^{1m}, —NO₂, —NR^{1l}R^{1m}, —NR^{1l}COR^{1m}, —NR¹′CO₂R^{1m}, —NR^{1l}CONR^{1m}R¹ⁿ, or —NR^{1l}SO₂R^{1m}; wherein each of said —C₁₋₈alkyl, —C₂₋₈alkenyl, —C₂₋₈alkynyl, cycloalkyl, heterocyclyl, aryl or heteroaryl is optionally substituted with at least one substituent halogen, hydroxy, —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl
R^{1l}, R^{1m} and R¹ⁿ are each independently selected from hydrogen, —C₁₋₈alkyl, —C₂₋₈alkenyl, —C₂₋₈ alkynyl, cycloalkyl, heterocyclyl, aryl or heteroaryl, wherein each of said —C₁₋₈alkyl, —C₂₋₈alkenyl, —C₂₋₈ alkynyl, cycloalkyl, heterocyclyl, aryl or heteroaryl is optionally substituted with at least one substituent halogen, hydroxy, —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R² is hydrogen, —C₁₋₈alkyl, —C₂₋₈alkenyl, —C₂₋₈alkynyl, cycloalkyl, heterocyclyl, aryl or heteroaryl; wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{2a};
R^{2a} is each independently selected from hydrogen, halogen, —C₁₋₈alkyl, —C₂₋₈alkenyl, —C₂₋₈alkynyl, cycloalkyl, heterocyclyl, aryl, heteroaryl, oxo, —CN, —OR^{2b}, —SO₂R^{2b}, —SO₂NR^{2b}R^{2c}, —COR^{2b}, —CO₂R^{2b}, —CONR^{2b}R^{2c}, —NO₂, —NR^{2b}R^{2c}, —NR^{2b}COR^{2c}, —NR^{2b}CO₂R^{2c}, —NR^{2b}CONR^{2c}R^{2d} or —NR^{2b}SO₂R^{2c}; wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{2e}; or
two R^{2a}, together with the atom(s) to which they are attached, form a 3- to 12-membered ring, said ring comprising 0, 1 or 2 heteroatom(s) independently selected from nitrogen, oxygen, or optionally oxidized sulfur as ring member(s), said ring is optionally substituted with at least one substituent R^{2f};
R^{2b}, R^{2c} and R^{2d} are each independently selected from hydrogen, —C₁₋₈alkyl, —C₂₋₈alkenyl, —C₂₋₈ alkynyl, cycloalkyl, heterocyclyl, aryl or heteroaryl, wherein each of said —C₁₋₈alkyl, —C₂₋₈alkenyl, —C₂₋₈ alkynyl, cycloalkyl, heterocyclyl, aryl or heteroaryl is optionally substituted with at least one substituent halogen, hydroxy, —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R^{2e} and R^{2f} are each independently selected from hydrogen, halogen, —C₁₋₈alkyl, —C₂₋₈alkenyl, —C₂₋₈ alkynyl, —C₁₋₈alkoxy, cycloalkyl, heterocyclyl, aryl, heteroaryl; wherein each of said —C₁₋₈alkyl, —C₂₋₈ alkenyl, —C₂₋₈alkynyl, —C₁₋₈alkoxy, cycloalkyl, heterocyclyl, aryl or heteroaryl is optionally substituted with at least one substituent halogen, hydroxy, —C₁₋₈alkyl, -haloC₁₋₈alkyl, —C₁₋₈alkoxy, —C₂₋₈alkenyl, —C₂₋₈ alkynyl, cycloalkyl, halocycloalkyl, heterocyclyl, haloheterocyclyl, aryl, haloaryl, heteroaryl or haloheteroaryl;
R³ and R⁴ are each independently hydrogen, halogen, —C₁₋₈alkyl, —C₂₋₈alkenyl, —C₂₋₈alkynyl, cycloalkyl, heterocyclyl, aryl, heteroaryl, oxo, —CN, —OR^{3a}, —SO₂R^{3a}, —SO₂NR^{3a}R^{3b}, —COR^{3a}, —CO₂R^{3a}, —CONR^{3a}R^{3b}, —NR^{3a}R^{3b}, —NR^{3a}COR^{3b}, —NR^{3a}CO₂R^{3b}, —NR^{3a}CONR^{3b}R^{3c}, or —NR^{3a}SO₂R^{3b}; wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{3d};
R^{3a}, R^{3b} and R^{3c} are each independently selected from hydrogen, —C₁₋₈alkyl, —C₂₋₈alkenyl, —C₂₋₈ alkynyl, cycloalkyl, heterocyclyl, aryl or heteroaryl, wherein each of said —C₁₋₈alkyl, —C₂₋₈alkenyl, —C₂₋₈ alkynyl, cycloalkyl, heterocyclyl, aryl or heteroaryl is optionally substituted with at least one substituent R^{3f}; or
(R^{3a} and R^{3b}), (R^{3b} and R^{3c}) or (R^{3a} and R^{3c}), together with the atom(s) to which they are attached, form a 3- to 12-membered ring, said ring comprising 0, 1 or 2 heteroatom(s) independently selected from nitrogen, oxygen, or optionally oxidized sulfur as ring member(s), said ring is optionally substituted with at least one substituent R^{3f};
R^{3d} and R^{3f} are each independently selected from hydrogen, halogen, —C₁₋₈alkyl, —C₂₋₈alkenyl, —C₂₋₈ alkynyl, cycloalkyl, heterocyclyl, aryl, heteroaryl, oxo, —CN, —OR^{3g}, —SO₂R^{3g}, —SO₂NR^{3g}R^{3h}, —COR^{3g}, —CO₂R^{3g}, —CONR^{3g}R^{3h}, —NO₂, —NR^{3g}R^{3h}, —NR^{3g}COR^{3h}, —NR^{3g}CO₂R^{3h}, —NR^{3g}CONR^{3h}, R³ⁱ, or —NR^{3g}SO₂R^{3h}; wherein each of said —C₁₋₈alkyl, —C₂₋₈alkenyl, —C₂₋₈alkynyl, cycloalkyl, heterocyclyl, aryl or heteroaryl is optionally substituted with at least one substituent halogen, hydroxy, —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R^{3g}, R^{3h} and R³ⁱ are each independently selected from hydrogen, —C₁₋₈alkyl, —C₂₋₈alkenyl, —C₂₋₈ alkynyl, cycloalkyl, heterocyclyl, aryl or heteroaryl, wherein each of said —C₁₋₈alkyl, —C₂₋₈alkenyl, —C₂₋₈ alkynyl, cycloalkyl, heterocyclyl, aryl or heteroaryl is optionally substituted with at least one substituent halogen, hydroxy, —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R⁵ is halogen, —C₁₋₈alkyl, —C₂₋₈alkenyl, —C₂₋₈alkynyl, cycloalkyl, heterocyclyl, aryl, heteroaryl, oxo, —CN, —OR^{5a}, —SR^{5a}, —SO₂R^{5a}, —P(O)R^{5a}R^{5b}, —SO₂NR^{5a}R^{5b}, —COR^{5a}, —CO₂R^{5a}, —CONR^{5a}R^{5b}, —NR^{5a}R^{5b}, —NR^{5a}COR^{5b}, —NR^{5a}CO₂R^{5b}, —NR^{5a}CONR^{5b}R^{5c}, or —NR^{5a}SO₂R^{5b}; wherein each of said —C₁₋₈alkyl, —C₂₋₈ alkenyl, —C₂₋₈alkynyl, cycloalkyl, heterocyclyl, aryl or heteroaryl is optionally substituted with at least one substituent R^{5d};
R^{5a}, R^{5b} and R^{5c} are each independently selected from hydrogen, —C₁₋₈alkyl, —C₂₋₈alkenyl, —C₂₋₈ alkynyl, cycloalkyl, heterocyclyl, aryl or heteroaryl, wherein each of said —C₁₋₈alkyl, —C₂₋₈alkenyl, —C₂₋₈ alkynyl, cycloalkyl, heterocyclyl, aryl or heteroaryl is optionally substituted with at least one substituent R^{5f}; or
(R^{5a} and R^{5b}), (R^{5b} and R^{5c}) or (R^{5a} and R^{5c}), together with the atom(s) to which they are attached, form a 3- to 12-membered ring, said ring comprising 0, 1 or 2 heteroatom(s) independently selected from nitrogen, oxygen, or optionally oxidized sulfur as ring member(s), said ring is optionally substituted with at least one substituent R^{5f};
R^{5d} and R^{5f} are each independently selected from hydrogen, halogen, —C₁₋₈alkyl, —C₂₋₈alkenyl, —C₂₋₈ alkynyl, cycloalkyl, heterocyclyl, aryl, heteroaryl, oxo, —CN, —OR^{5g}, —SO₂R^{5g}, —SO₂NR^{5g}R^{5h}, —COR^{5g}, —CO₂R^{5g}, —CONR^{5g}R^{5h}, —NO₂, —P(O)R^{5g}R^{5h}, —NR^{5g}R^{5h}, —NR^{5g}COR^{5h}, —NR^{5g}CO₂R^{5h}, —NR^{5g}CONR^{5h}R⁵ⁱ or —NR^{5g}SO₂R^{5h}; wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{5j}; or
two R^{5d} or two R^{5f}, together with the atom(s) to which they are attached, form a 3- to 12-membered ring, said ring comprising 0, 1 or 2 heteroatom(s) independently selected from nitrogen, oxygen, or optionally oxidized sulfur as ring member(s), said ring is optionally substituted with at least one substituent R^{5k};
R^{5g}, R^{5h} and R⁵ⁱ are each independently selected from hydrogen, —C₁₋₈alkyl, —C₂₋₈alkenyl, —C₂₋₈ alkynyl, cycloalkyl, heterocyclyl, aryl or heteroaryl, wherein each of said —C₁₋₈alkyl, —C₂₋₈alkenyl, —C₂₋₈ alkynyl, cycloalkyl, heterocyclyl, aryl or heteroaryl is optionally substituted with at least one substituent halogen, hydroxy, —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl; or
(R^{5g} and R^{5h}), (R^{5h} and R⁵ⁱ) or (R^{5g} and R⁵ⁱ), together with the atom(s) to which they are attached, form a 3- to 12-membered ring, said ring comprising 0, 1 or 2 heteroatom(s) independently selected from nitrogen, oxygen, or optionally oxidized sulfur as ring member(s), said ring is optionally substituted with at least one substituent halogen, hydroxy, —C₁₋₈alkyl, -haloC₁₋₈alkyl, —C₁₋₈alkoxy, —C₂₋₈ alkenyl, —C₂₋₈alkynyl, cycloalkyl, halocycloalkyl, heterocyclyl, haloheterocyclyl, aryl, haloaryl, heteroaryl or haloheteroary;
R^{5j} and R^{5k} are each independently selected from hydrogen, halogen, —C₁₋₈alkyl, —C₂₋₈alkenyl, —C₂₋₈ alkynyl, cycloalkyl, heterocyclyl, aryl, heteroaryl, oxo, —CN, —OR^{5l}, —SO₂R^{5l}, —SO₂NR^{5l}R^{5m}, —COR^{5l}, —CO₂R^{5l}, —CONR^{5l}R^{5m}, —NO₂, —NR^{5l}R^{5m}, —NR^{5l}COR^{5l}, —NR^{5l}CO₂R^{5m}, —NR^{5l}CONR^{5m}R⁵ⁿ, or —NR^{5l}SO₂R^{5m}; wherein each of said —C₁₋₈alkyl, —C₂₋₈alkenyl, —C₂₋₈alkynyl, cycloalkyl, heterocyclyl, aryl or heteroaryl is optionally substituted with at least one substituent halogen, hydroxy, —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl
R^{5l}, R^{5m} and R⁵ⁿ are each independently selected from hydrogen, —C₁₋₈alkyl, —C₂₋₈alkenyl, —C₂₋₈ alkynyl, cycloalkyl, heterocyclyl, aryl or heteroaryl, wherein each of said —C₁₋₈alkyl, —C₂₋₈alkenyl, —C₂₋₈ alkynyl, cycloalkyl, heterocyclyl, aryl or heteroaryl is optionally substituted with at least one substituent halogen, hydroxy, —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R^{z}, at each of its occurrences, is independently hydrogen, halogen, —C₁₋₈alkyl, —C₂₋₈alkenyl, —C₂₋₈ alkynyl, cycloalkyl, heterocyclyl, aryl, heteroaryl, oxo, —CN, —OR^{za}, —SO₂R^{za}, —SO₂NR^{za}R^{zb}, —COR^{za}, —CO₂R^{za}, —CONR^{za}R^{zb}, —NR^{za}R^{zb}, —NR^{za}COR^{zb}, —NR^{za}CO₂R^{zb}, —NR^{za}CONR^{zb}R^{zc}, or —NR^{za}SO₂R^{zb}; wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{zd};
R^{za}, R^{zb} and R^{zc} are each independently selected from hydrogen, —C₁₋₈alkyl, —C₂₋₈alkenyl, —C₂₋₈ alkynyl, cycloalkyl, heterocyclyl, aryl or heteroaryl, wherein each of said —C₁₋₈alkyl, —C₂₋₈alkenyl, —C₂₋₈ alkynyl, cycloalkyl, heterocyclyl, aryl or heteroaryl is optionally substituted with at least one substituent R^{zf}; or
(R^{za} and R^{zb}), (R^{zb} and R^{zc}) or (R^{za} and R^{zc}), together with the atom(s) to which they are attached, form a 3- to 12-membered ring, said ring comprising 0, 1 or 2 heteroatom(s) independently selected from nitrogen, oxygen, or optionally oxidized sulfur as ring member(s), said ring is optionally substituted with at least one substituent R^{zf};
R^{zd} and R^{zf} are each independently selected from hydrogen, halogen, —C₁₋₈alkyl, —C₂₋₈alkenyl, —C₂₋₈ alkynyl, cycloalkyl, heterocyclyl, aryl, heteroaryl, oxo, —CN, —OR^{zg}, —SO₂R^{zg}, —SO₂NR^{zg}R^{zh}, —COR^{zg}, —CO₂R^{zg}, —CONR^{zg}R^{zh}, —NO₂, —NR^{zg}R^{zh}, —NR^{zg}COR^{zh}, —NR^{zg}CO₂R^{zh}, —NR^{zg}CONR^{zh}R^{zi}, or —NR^{zg}SO₂R^{zh}; wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{zj}; or
two R^{zd} or two R^{zf}, together with the atom(s) to which they are attached, form a 3- to 12-membered ring, said ring comprising 0, 1 or 2 heteroatom(s) independently selected from nitrogen, oxygen, or optionally oxidized sulfur as ring member(s), said ring is optionally substituted with at least one substituent R^{zk};
R^{zg}, R^{zh} and R^{zi} are each independently selected from hydrogen, —C₁₋₈alkyl, —C₂₋₈alkenyl, —C₂₋₈ alkynyl, cycloalkyl, heterocyclyl, aryl or heteroaryl, wherein each of said —C₁₋₈alkyl, —C₂₋₈alkenyl, —C₂₋₈ alkynyl, cycloalkyl, heterocyclyl, aryl or heteroaryl is optionally substituted with at least one substituent halogen, hydroxy, —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R^{zj} and R^{zk} are each independently selected from hydrogen, halogen, —C₁₋₈alkyl, —C₂₋₈alkenyl, —C₂₋₈ alkynyl, —C₁₋₈alkoxy, cycloalkyl, heterocyclyl, aryl, heteroaryl, oxo, —CN; wherein each of said —C₁₋₈alkyl, —C₂₋₈alkenyl, —C₂₋₈alkynyl, —C₁₋₈alkoxy, cycloalkyl, heterocyclyl, aryl or heteroaryl is optionally substituted with at least one substituent halogen, hydroxy, —C₁₋₈alkyl, -haloC₁₋₈alkyl, —C₁₋₈alkoxy, —C₂₋₈ alkenyl, —C₂₋₈alkynyl, cycloalkyl, halocycloalkyl, heterocyclyl, haloheterocyclyl, aryl, haloaryl, heteroaryl or haloheteroaryl;
n1 is 0, 1, 2, 3, 4, 5, 6, 7 or 8.

2. The compound of claim 1, wherein the compound is selected from formula (IIa), (IIb), (IIc), (IId) or (IIe): 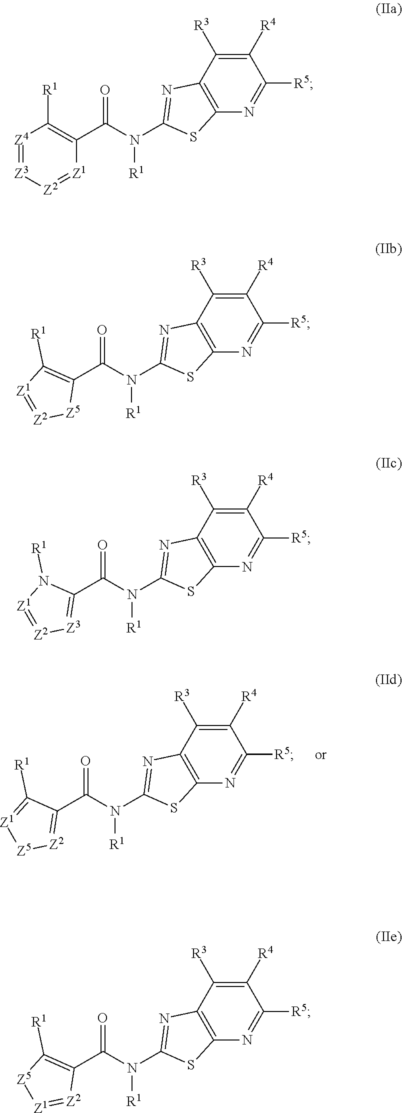
wherein, Z¹, Z², Z³ and Z⁴ are each independently N or CR^{z}; and Z⁵ is NR^{z}, O or S.

3. The compound of claim 1, wherein the compound is selected from formula (IIIa), (IIIb), (IIIc), (IIId), (IIIe), (IIIf), (IIIg), (IIIh), (IIIi), (IIIj) or (IIIk):

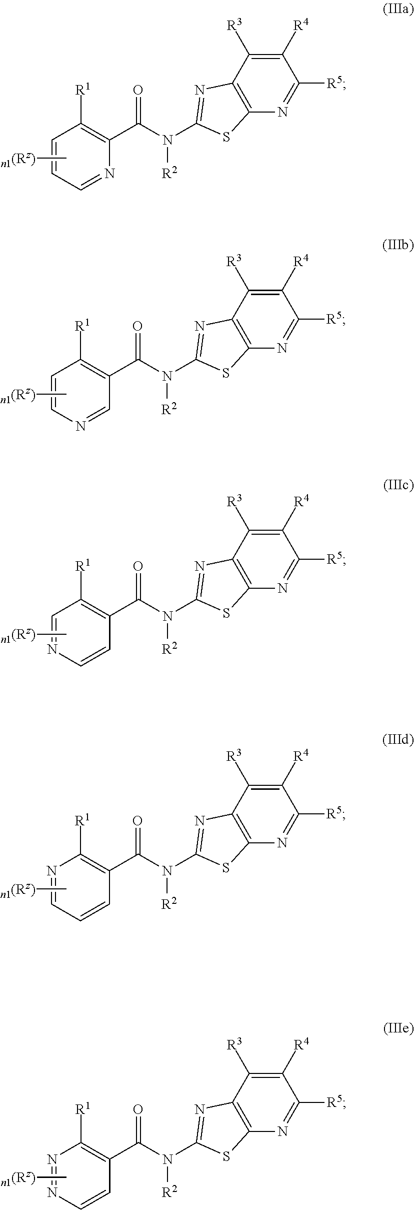

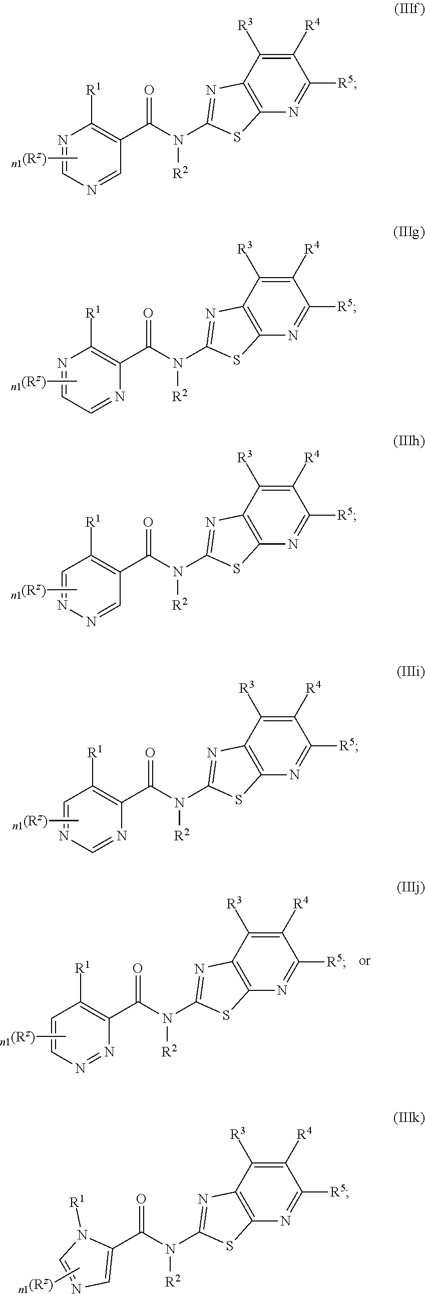

4. The compound of claim 1, wherein the compound is selected from formula (IVa), (IVb), (IVc) or (IVd): 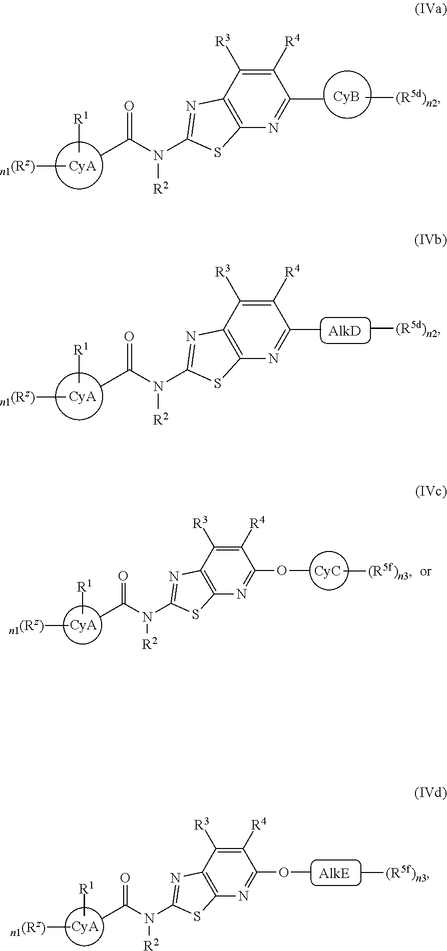
wherein,
CyB and CyC are each independently cycloalkyl, heterocyclyl, aryl or heteroaryl;
AlkD and AlkE are each —C₁₋₈alkyl, —C₂₋₈alkenyl or —C₂₋₈alkynyl; and
n2 and n3 are each independently 0, 1, 2, 3, 4, 5, 6, 7 or 8.

5. The compound of claim 4, wherein the compound is selected from formula (Va), (Vb), (Vc), (Vd):

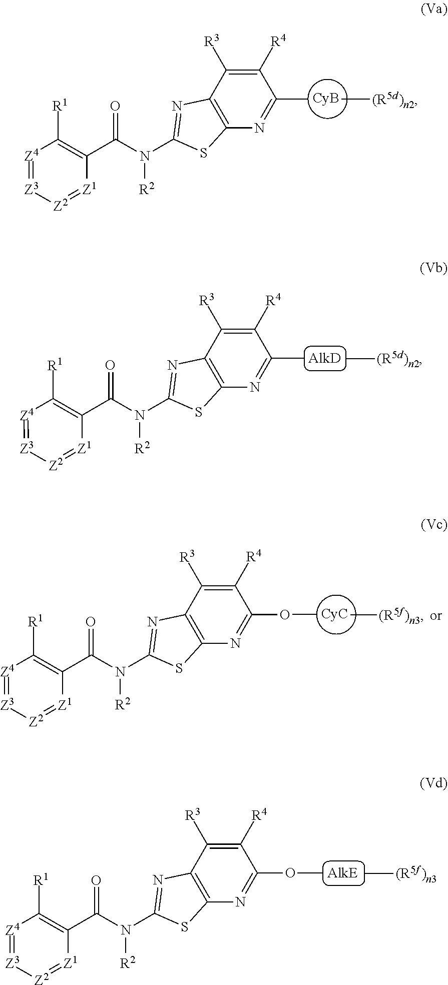

6. (canceled)

7. The compound of claim 1, wherein ring CyA is a 5- or 6-membered heteroaromatic ring comprising 1 to 2 heteroatoms selected from N, O, or S as ring member(s).

8. (canceled)

9. The compound of claim 1, wherein R¹ is cyclopropyl, cyclobutyl, cyclopentyl, cyclohexyl, cycloheptyl, cyclooctyl, morpholinyl, piperidinyl, piperazinyl, dihydropyridinyl, dihydropiperazinyl, phenyl, indazolyl, benzodioxolyl, benzooxazinyl, dihydrobenzooxazinyl, indolyl, benzoimidazolyl, benzooxazolyl, benzofuranyl, quinolinyl or isoquinolinyl; wherein each of said cyclopropyl, cyclobutyl, cyclopentyl, cyclohexyl, cycloheptyl, cyclooctyl, morpholinyl, piperidinyl, piperazinyl, dihydropyridinyl, dihydropiperazinyl, phenyl, indazolyl, benzodioxolyl, benzooxazinyl, dihydrobenzooxazinyl, indolyl, benzoimidazolyl, benzooxazolyl, benzofuranyl, quinolinyl or isoquinolinyl is optionally substituted with at least one substituent R^{1d};
R^{1d} is each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 6-membered heterocyclyl, aryl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl, heteroaryl, oxo, —CN, —OR^{1g}, —SO₂R^{1g}, —SO₂NR^{1g}R^{1h}, —COR^{1g}, —CO₂R^{1h}, —CONR^{1g}R^{1h}, —NO₂, —NR^{1g}R^{1h}, —NR^{1g}COR^{1h}, —NR^{1g}CO₂R^{1h}, —NR^{1g}CONR^{1h}R¹ⁱ, or —NR^{1g}SO₂R^{1h}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl or heteroaryl is optionally substituted with at least one substituent R^{1j}; or
two R^{1d} together with the atom(s) to which they are attached, form a 3-, 4-, 5-, 6-, 7- or 8-membered ring, said ring comprising 0, 1 or 2 heteroatom(s) independently selected from nitrogen, oxygen, or optionally oxidized sulfur as ring member(s), said ring is optionally substituted with at least one substituent R^{1k};
R^{1g}, R^{1h} and R¹ⁱ are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl or heteroaryl is optionally substituted with at least one substituent —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈ alkynyl, cyclopropyl, cyclobutyl, cyclopentyl, cyclohexyl, cycloheptyl, cyclooctyl, halocycloalkyl, heterocyclyl, haloheterocyclyl, aryl, haloaryl, heteroaryl or haloheteroaryl;
R^{1j} and R^{1k} are each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl, heteroaryl, oxo, —CN, —OR^{1l}, —SO₂R^{1l}, —SO₂NR^{1l}R^{1m}, —COR^{1l}, —CO₂R^{1l}, —CONR^{1l}R^{1m}, —NO₂, —NR^{1l}R^{1m}, —NR^{1l}COR^{1m}, —NR^{1l}CO₂R^{1m}, —NR^{1l}CONR^{1m}R¹ⁿ or —NR^{1l}SO₂R^{1m}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl or heteroaryl is optionally substituted with at least one substituent —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, aryl, haloaryl, heteroaryl or haloheteroaryl;
R^{1l}, R^{1m} and R¹ⁿ are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl or heteroaryl is optionally substituted with at least one substituent —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈ alkynyl, cyclopropyl, cyclobutyl, cyclopentyl, cyclohexyl, cycloheptyl, cyclooctyl, halocycloalkyl, heterocyclyl, haloheterocyclyl, aryl, haloaryl, heteroaryl or haloheteroaryl.

10. (canceled)

11. The compound of claim 1, wherein R¹ is

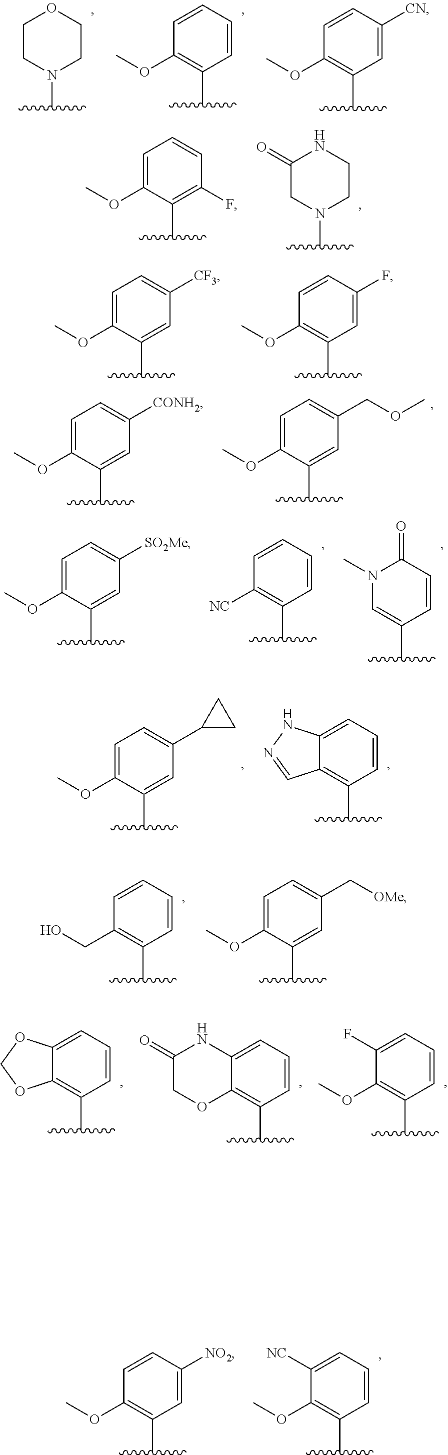

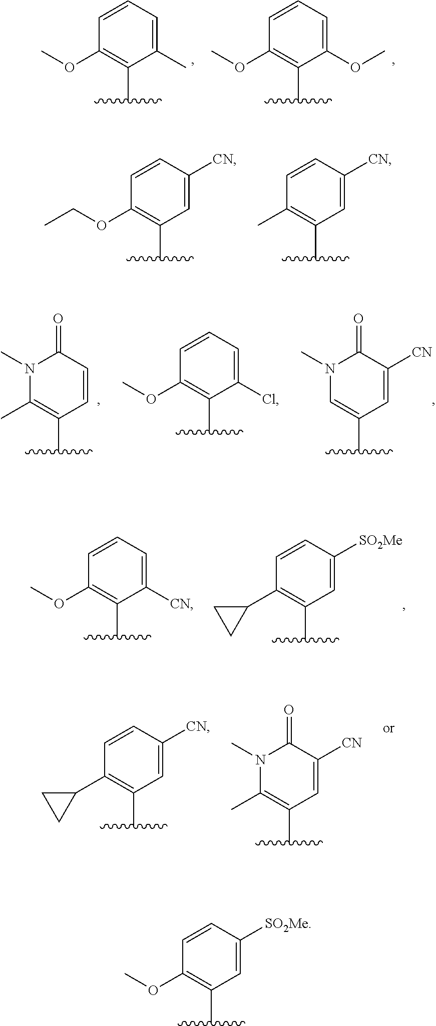

12. The compound of claim 1, wherein
R^{z}, at each of its occurrences, is independently hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, oxo, —CN, —OR^{z}, —SO₂R^{za}, —SO₂NR^{za}R^{zb}, —COR^{za}, —CO₂R^{za}, —CONR^{za}R^{zb}, —NR^{za}R^{zb}, —NR^{za}COR^{zb}, —NR^{za}CO₂R^{zb}, —NR^{za}CONR^{zb}R^{zc} or —NR^{za}SO₂R^{zb}; wherein each of said methyl, ethyl, propyl, butyl, pentyl, hexyl, heptyl, octyl, —C₂₋₈ alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent R^{zd};
R^{za}, R^{zb} and R^{zc} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent R^{zf}; or
(R^{za} and R^{zb}), (R^{zb} and R^{zc}) or (R^{za} and R^{zc}), together with the atom(s) to which they are attached, form a 3-, 4-, 5-, 6-, 7- or 8-membered ring, said ring comprising 0, 1 or 2 heteroatom(s) independently selected from nitrogen, oxygen, or optionally oxidized sulfur as ring member(s), said ring is optionally substituted with at least one substituent R^{zf};
R^{zd} and R^{zf} are each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, oxo, —CN, —OR^{zg}, —SO₂R^{zg}, —SO₂NR^{zg}R^{zh}, —COR^{zg}, —CO₂R^{zg}, —CONR^{zg}R^{zh}, —NO₂, —NR^{zg}R^{zh}, —NR^{zg}COR^{zh}, —NR^{zg}CO₂R^{zh}, —NR^{zg}CONR^{zh}R^{zi}, or —NR^{zg}SO₂R^{zh}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent R^{zj}; or
two R^{zd} or two R^{zf}, together with the atom(s) to which they are attached, form a 3-, 4-, 5-, 6-, 7- or 8-membered ring, said ring comprising 0, 1 or 2 heteroatom(s) independently selected from nitrogen, oxygen, or optionally oxidized sulfur as ring member(s), said ring is optionally substituted with at least one substituent R^{zk};
R^{zg}, R^{zh} and R^{zi} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent halogen, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈ alkynyl, cyclopropyl, cyclobutyl, cyclopentyl, cyclohexyl, cycloheptyl, cyclooctyl, halocycloalkyl, heterocyclyl, haloheterocyclyl, aryl, haloaryl, heteroaryl or haloheteroaryl;
R^{zj} and R^{zk} are each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, hepthoxy, octoxy, cyclopropyl, cyclobutyl, cyclopentyl, cyclohexyl, cycloheptyl, cyclooctyl, heterocyclyl, phenyl, heteroaryl, oxo, —CN; wherein each of said methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, hepthoxy, octoxy, cyclopropyl, cyclobutyl, cyclopentyl, cyclohexyl, cycloheptyl, cyclooctyl, heterocyclyl, phenyl or heteroaryl is optionally substituted with at least one substituent —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl.

13. (canceled)

14. The compound of claim 1, wherein R^{z}, at each of its occurrences, is independently H, —CN, —CH₃, —Cl, —CH₂CN, —CF₃, —F, —COOH or —CH₂OH.

15. The compound of claim 1, wherein the 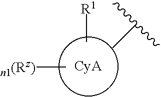
moiety is

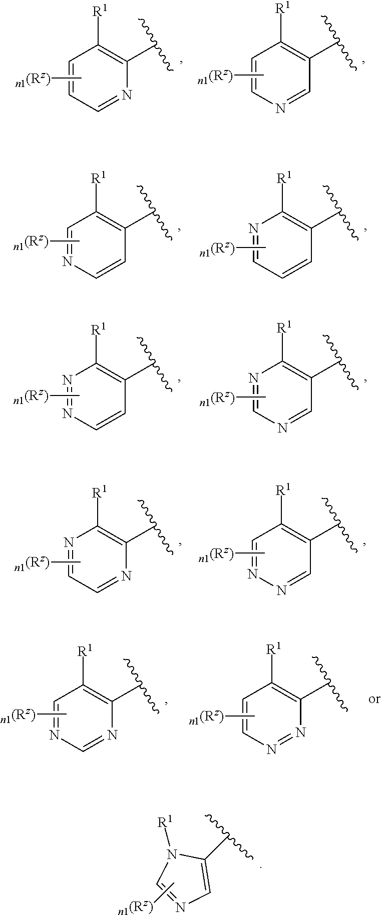

16. The compound of claim 1, wherein the 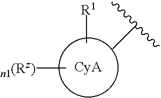
moiety is

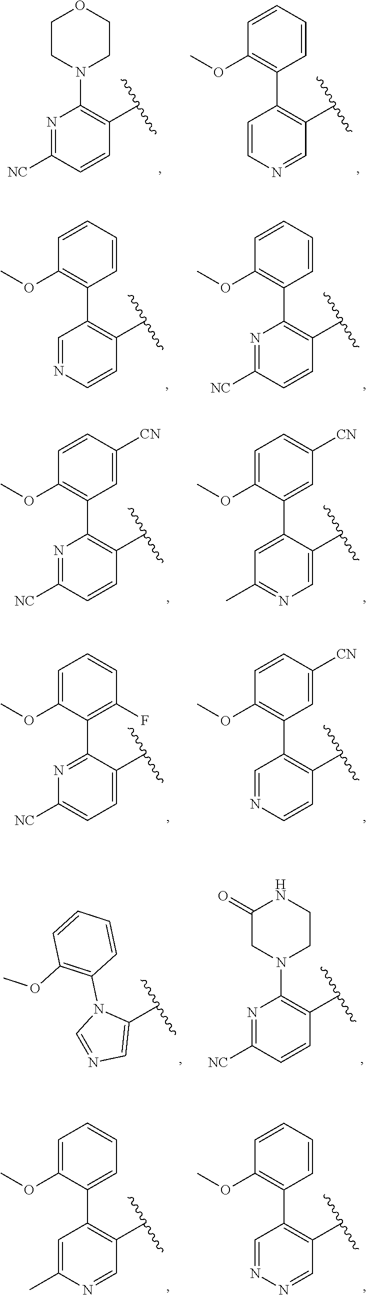

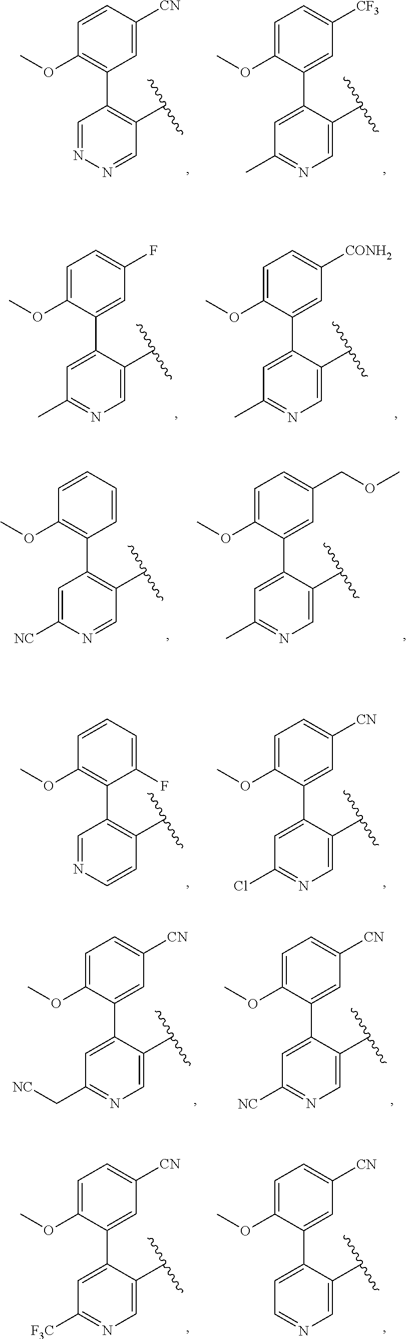

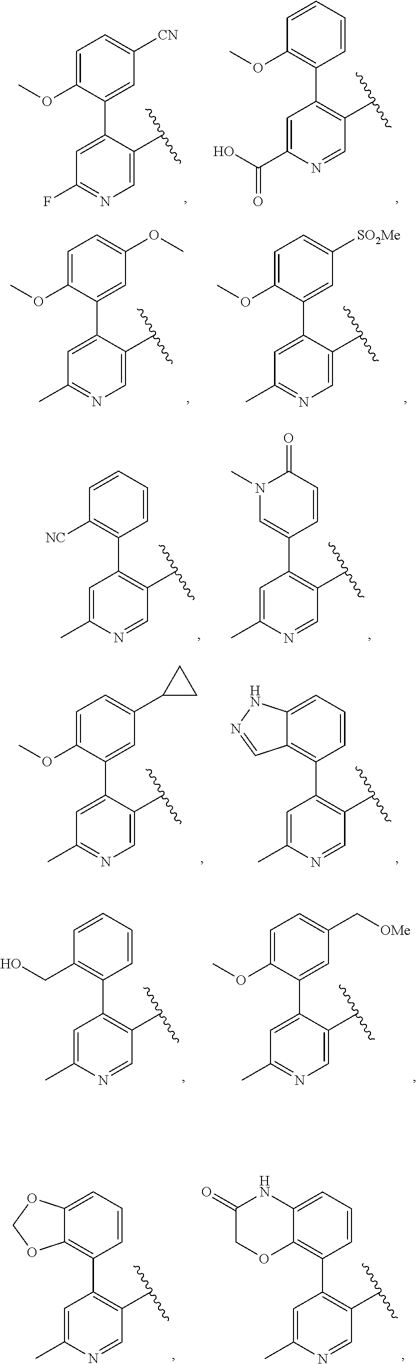

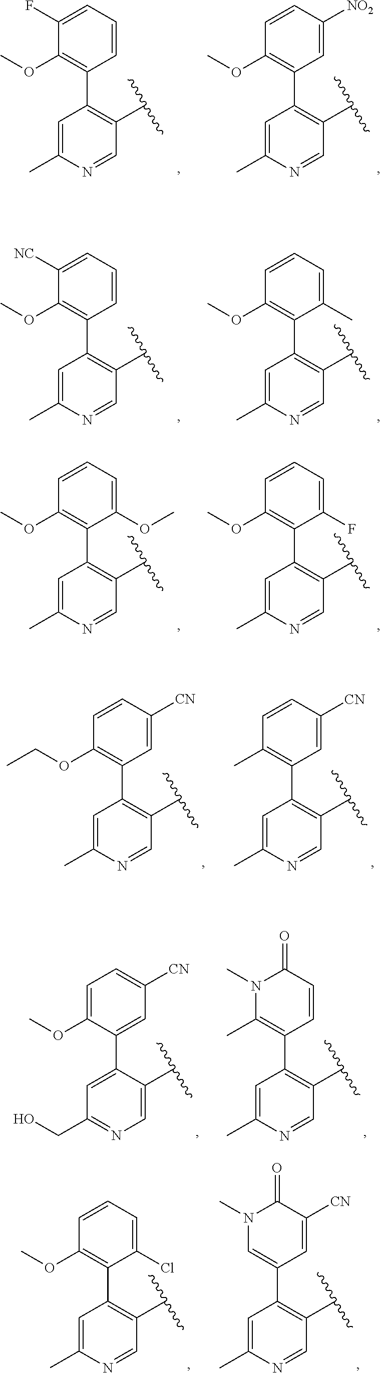

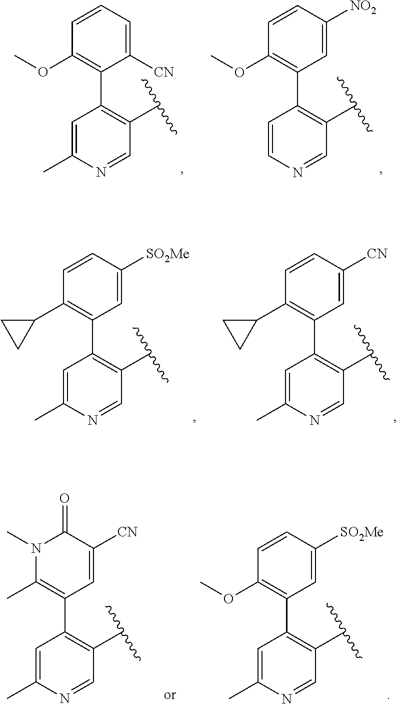 [SMILES: *c1cnc(C)cc1-c1c(C#N)cccc1OC ; *c1cnccc1-c1cc([N+](=O)[O-])ccc1OC ; *c1cnc(C)cc1-c1cc(S(C)(=O)=O)ccc1C1CC1 ; *c1cnc(C)cc1-c1cc(C#N)ccc1C1CC1 ; *c1cnc(C)cc1-c1cc(C#N)c(=O)n(C)c1C ; *c1cnc(C)cc1-c1cc(S(C)(=O)=O)ccc1OC]

17. The compound of claim 1, wherein R² is hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl or 5- to 10-membered heteroaryl; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl or 5- to 10-membered heteroaryl is optionally substituted with at least one substituent R^{2a};
R^{2a} is each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl, 5- to 10-membered heteroaryl, oxo, —CN, —OR^{2b}, —SO₂R^{2b}, —SO₂NR^{2b}R^{2c}, —COR^{2b}, —CO₂R^{2b}, —CONR^{2b}R^{2c}, —NO₂, —NR^{2b}R^{2c}, —NR^{2b}COR^{2c}, —NR^{2b}CO₂R^{2c}, —NR^{2b}CONR^{2c}R^{2d}, or —NR^{2b}SO₂R^{2c}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl or 5- to 10-membered heteroaryl is optionally substituted with at least one substituent R^{2e}; or
two R^{2a}, together with the atom(s) to which they are attached, form a 3-, 4-, 5-, 6-, 7- or 8-membered ring, said ring comprising 0, 1 or 2 heteroatom(s) independently selected from nitrogen, oxygen, or optionally oxidized sulfur as ring member(s), said ring is optionally substituted with at least one substituent R^{2f};
R^{2b}, R^{2c} and R^{2d} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl or 5- to 10-membered heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl or 5- to 10-membered heteroaryl is optionally substituted with at least one substituent halogen, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, 5- to 8-membered heterocyclyl, haloheterocyclyl, phenyl, 5- to 10-membered heteroaryl or haloheteroaryl;
R^{2e} and R^{2f} are each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, hepthoxy, octoxy, cyclopropyl, cyclobutyl, cyclopentyl, cyclohexyl, cycloheptyl, cyclooctyl, 5- to 8-membered heterocyclyl, phenyl or 5- to 10-membered heteroaryl; wherein each of said methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, hepthoxy, octoxy, cyclopropyl, cyclobutyl, cyclopentyl, cyclohexyl, cycloheptyl, cyclooctyl, 5- to 8-membered heterocyclyl, phenyl or 5- to 10-membered heteroaryl is optionally substituted with at least one substituent —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl.

18. The compound of claim 1, wherein
R² is hydrogen.

19. The compound of claim 1, wherein R³ and R⁴ are each independently hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl, 5- to 10-membered heteroaryl, oxo, —CN, —OR^{3a}, —SO₂R^{3a}, —SO₂NR^{3a}R^{3b}, —COR^{3a}, —CO₂R^{3a}, —CONR^{3a}R^{3b}, —NR^{3a}R^{3b}, —NR^{3a}COR^{3b}, —NR^{3a}CO₂R^{3b}, —NR^{3a}CONR^{3b}R^{3c} or —R^{3a}SO₂R^{3b}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl or 5- to 10-membered heteroaryl is optionally substituted with at least one substituent R^{3d};
R^{3a}, R^{3b} and R^{3c} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent R^{3f}; or
(R^{3a} and R^{3b}), (R^{3b} and R^{3c}) or (R^{3a} and R^{3c}), together with the atom(s) to which they are attached, form a 3-, 4-, 5-, 6-, 7- or 8-membered ring, said ring comprising 0, 1 or 2 heteroatom(s) independently selected from nitrogen, oxygen, or optionally oxidized sulfur as ring member(s), said ring is optionally substituted with at least one substituent R^{3f};
R^{3d} and R^{3f} are each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl, 5- to 10-membered heteroaryl, oxo, —CN, —OR^{3g}, —SO₂R^{3g}, —SO₂NR^{3g}R^{3h}, —COR^{3g}, —CO₂R^{3g}, —CONR^{3g}R^{3h}, —NO₂, —NR^{3g}R^{3h}, —NR^{3g}COR^{3h}, —NR^{3g}CO₂R^{3h}, —NR^{3g}CONR^{3h}R³ⁱ, or —NR^{3g}SO₂R^{3h}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl, 5- to 10-membered heteroaryl is optionally substituted with at least one substituent halogen, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl;
R^{3g}, R^{3h} and R³ⁱ are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl, 5- to 10-membered heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl or 5- to 10-membered heteroaryl is optionally substituted with at least one substituent halogen, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl.

20. (canceled)

21. The compound of claim 1, wherein
R³ and R⁴ are each independently hydrogen.

22. The compound of claim 1, wherein R⁵ is —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl, 5- to 10-membered heteroaryl, oxo, —CN, —OR^{5a}, —SO₂R^{5a}, SO₂NR^{5a}R^{5b}, —COR^{5a}, —CO₂R^{5a}, —CONR^{5a}R^{5b}, —NR^{5a}R^{5b}, —NR^{5a}COR^{5b}, —NR^{5a}CO₂R^{5b}, —NR^{5a}CONR^{5b}R^{5c}, or —NR^{5a}SO₂R^{5b}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl or 5- to 10-membered heteroaryl is optionally substituted with at least one substituent R^{5d};
R^{5a}, R^{5b} and R^{5c} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl or 5- to 10-membered heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl, 5- to 10-membered heteroaryl is optionally substituted with at least one substituent R^{5f}; or
(R^{5a} and R^{5b}), (R^{5b} and R^{5c}) or (R^{5a} and R^{5c}), together with the atom(s) to which they are attached, form a 3-, 4-, 5-, 6-, 7- or 8-membered ring, said ring comprising 0, 1 or 2 heteroatom(s) independently selected from nitrogen, oxygen, or optionally oxidized sulfur as ring member(s), said ring is optionally substituted with at least one substituent R^{5f};
R^{5d} and R^{5f} are each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl, 5- to 10-membered heteroaryl, oxo, —CN, —OR^{5g}, —SO₂R^{5g}, —SO₂NR^{5g}R^{5h}, —COR^{5g}, —CO₂R^{5g}, —CONR^{5g}R^{5h}, —NO₂, —NR^{5g}R^{5h}, —NR^{5g}COR^{5h}, —NR^{5g}CO₂R^{5h}, —NR^{5g}CONR^{5h}R⁵ⁱ or —NR^{5g}SO₂R^{5h}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl or 5- to 10-membered heteroaryl is optionally substituted with at least one substituent R⁵; or
two R^{5d} or two R^{5f}, together with the atom(s) to which they are attached, form a 3-, 4-, 5-, 6-, 7- or 8-membered ring, said ring comprising 0, 1 or 2 heteroatom(s) independently selected from nitrogen, oxygen, or optionally oxidized sulfur as ring member(s), said ring is optionally substituted with at least one substituent R^{5k};
R^{5g}, R^{5h} and R⁵ⁱ are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl or 5- to 10-membered heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl or 5- to 10-membered heteroaryl is optionally substituted with at least one substituent halogen, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, 5- to 8-membered heterocyclyl, haloheterocyclyl, phenyl or 5- to 10-membered heteroaryl; or
(R^{5g} and R^{5h}), (R^{5h} and R⁵ⁱ) or (R^{5g} and R⁵ⁱ), together with the atom(s) to which they are attached, form a 3-, 4-, 5-, 6-, 7- or 8-membered ring, said ring comprising 0, 1 or 2 heteroatom(s) independently selected from nitrogen, oxygen, or optionally oxidized sulfur as ring member(s), said ring is optionally substituted with at least one substituent —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, 5- to 8-membered heterocyclyl, haloheterocyclyl, phenyl, haloaryl, heteroaryl or haloheteroary;
R^{5j} and R^{5k} are each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl, heteroaryl, oxo, —CN, —OR^{5l}, —SO₂R^{5l}, —SO₂NR^{5l}R^{5m}, —COR^{5l}, —CO₂R^{5l}, —CONR^{5l}R^{5m}, —NO₂, —NR^{5l}R^{5m}, —NR^{5l}COR^{5m}, —NR^{5l}CO₂R^{5m}, —NR^{5l}CONR^{5m}R⁵ⁿ, or —NR^{5l}SO₂R^{5m}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl or heteroaryl is optionally substituted with at least one substituent —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈ alkenyl, —C₂₋₈alkynyl, cyclopropyl, cyclobutyl, cyclopentyl, cyclohexyl, cycloheptyl, cyclooctyl, halocycloalkyl, 5- to 8-membered heterocyclyl, haloheterocyclyl, phenyl, haloaryl, heteroaryl or haloheteroaryl;
R^{5l}, R^{5m} and R⁵ⁿ are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl, or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 5- to 8-membered heterocyclyl, phenyl, or heteroaryl is optionally substituted with at least one substituent halogen, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, 5- to 8-membered heterocyclyl, haloheterocyclyl, phenyl, haloaryl, heteroaryl or haloheteroaryl.

23. (canceled)

24. The compound of claim 1, wherein R⁵ is

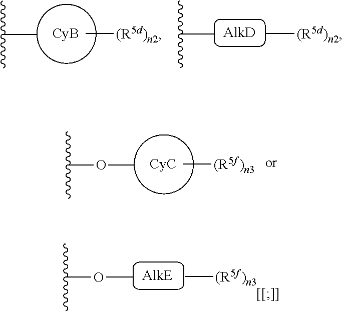

25. The compound of claim 1, wherein R⁵ is

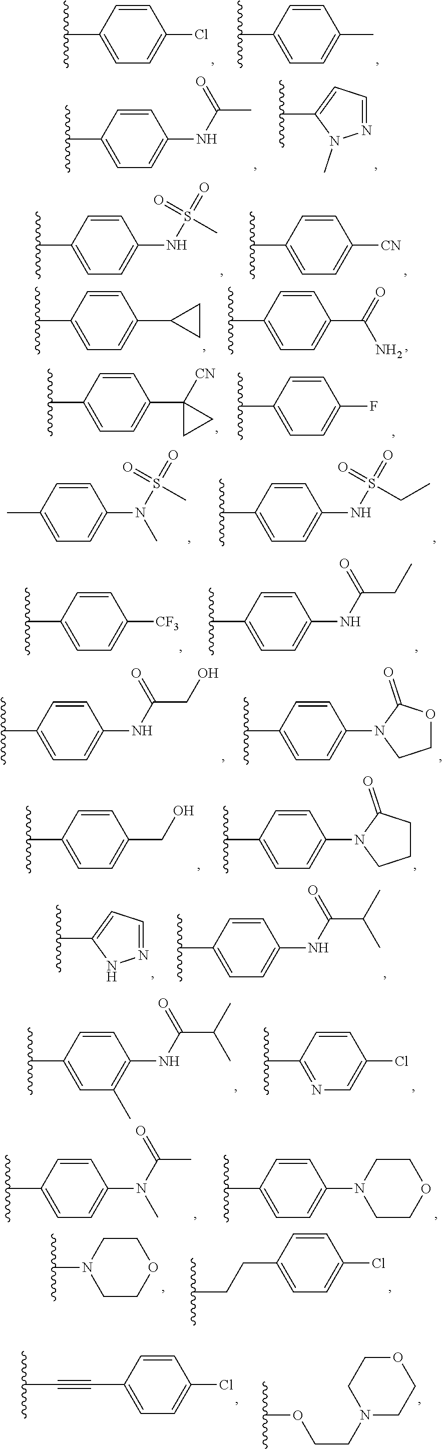

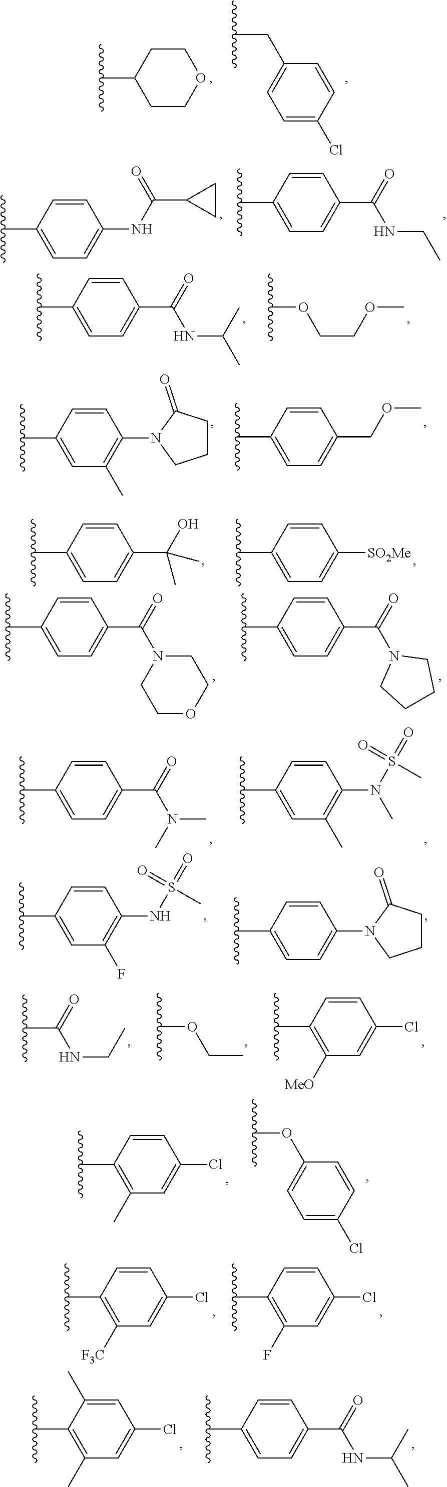

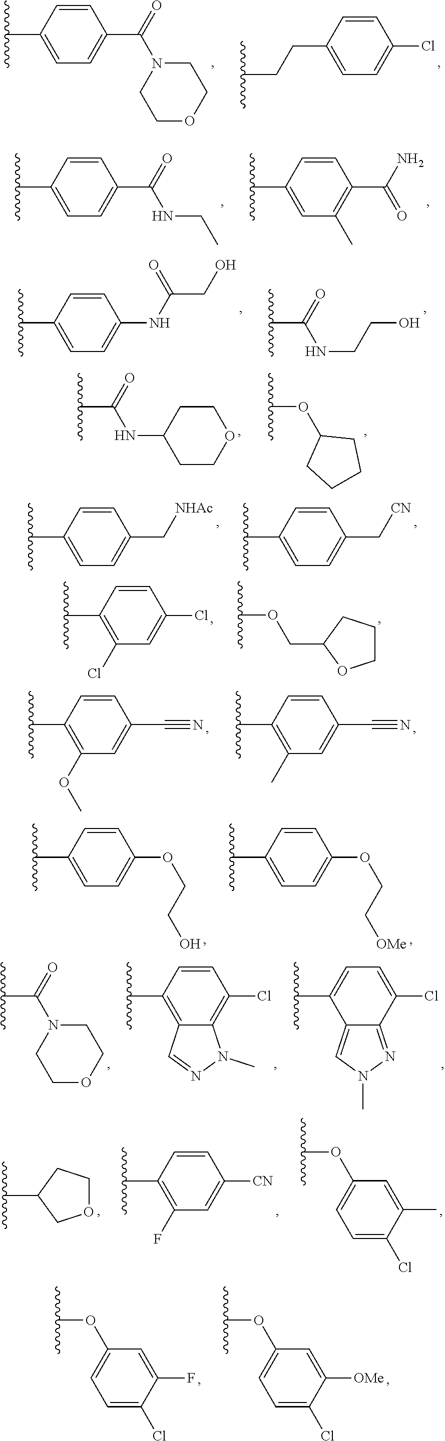

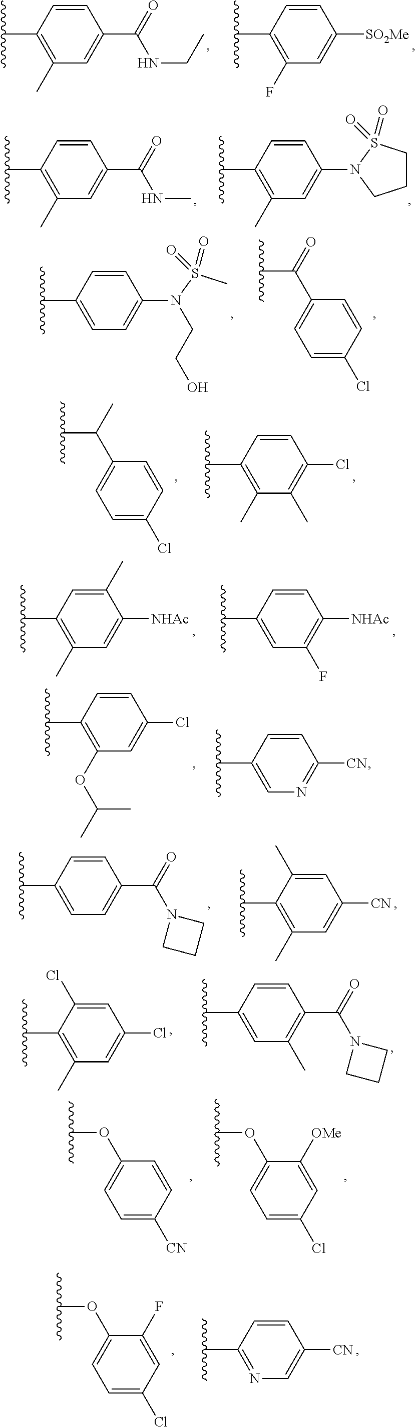

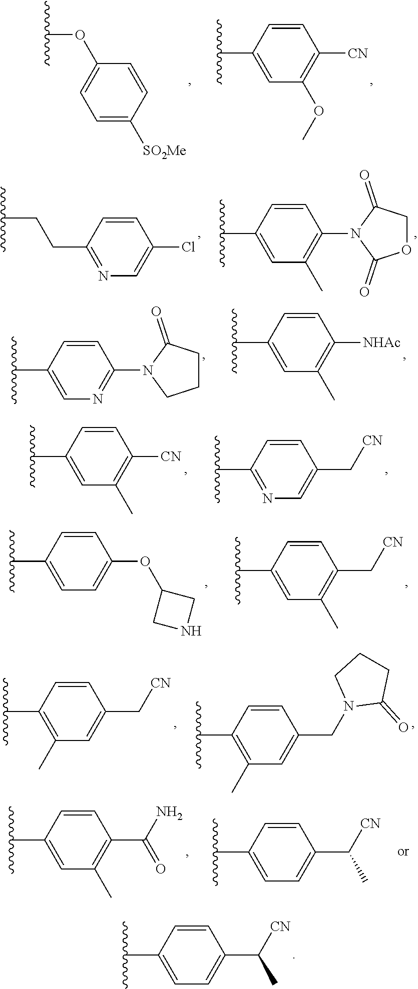

26. The compound of claim 1, wherein the compound is selected from 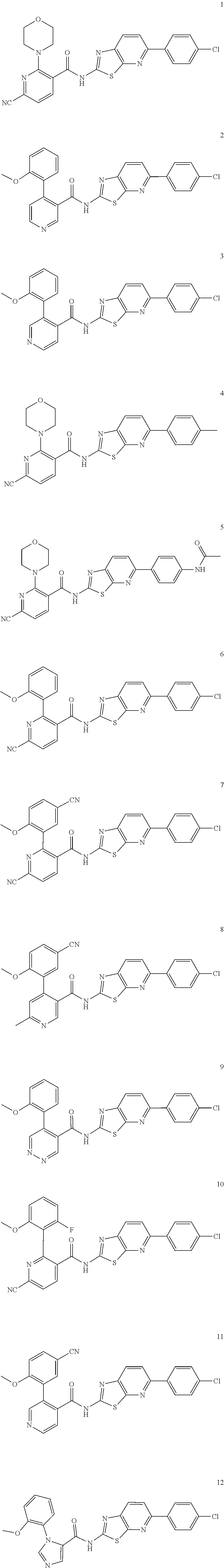 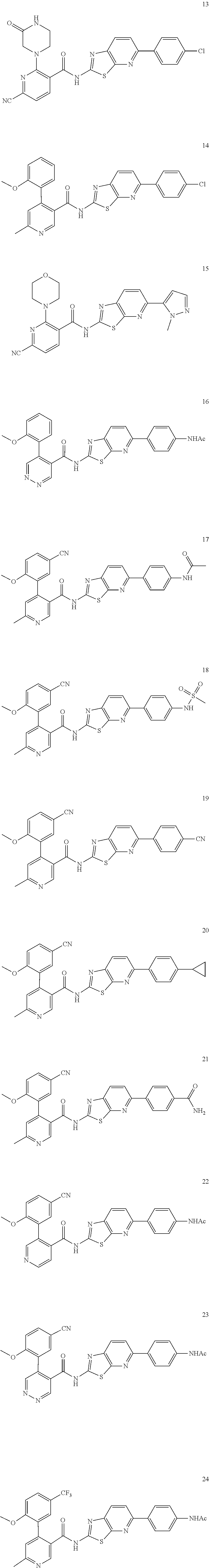 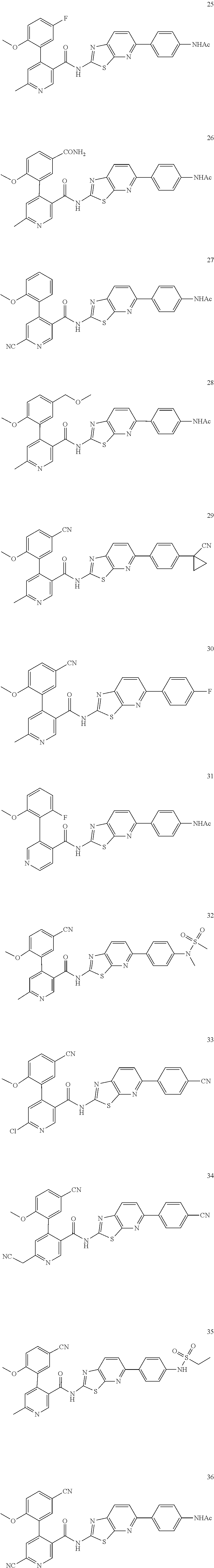 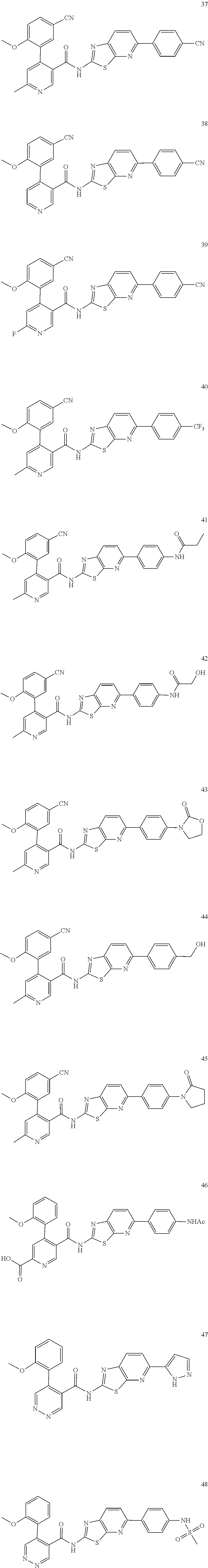 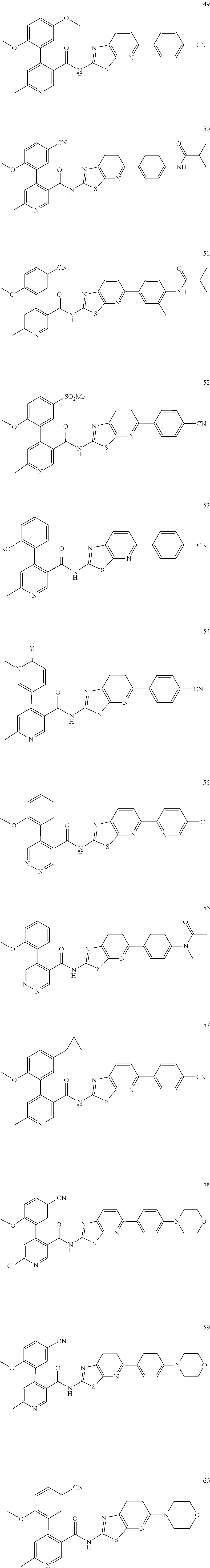 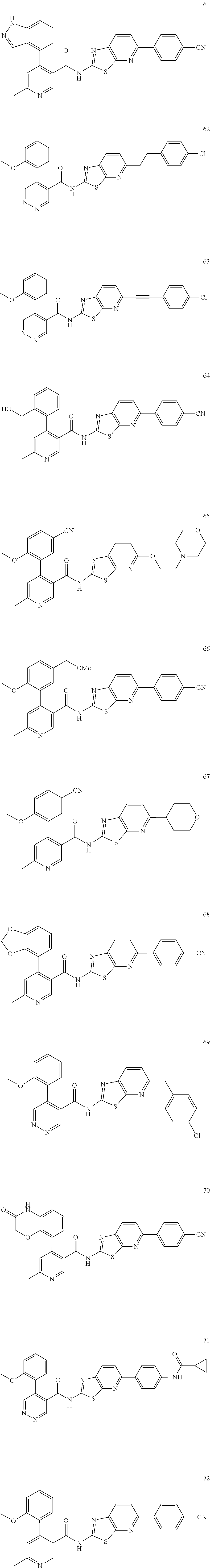 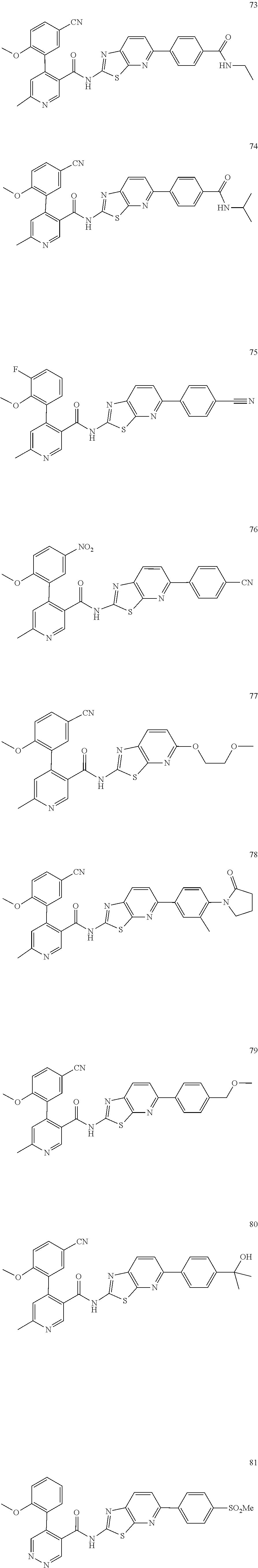 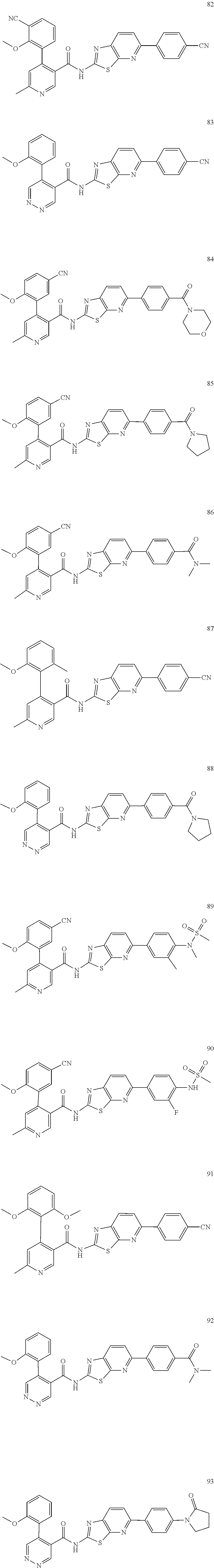 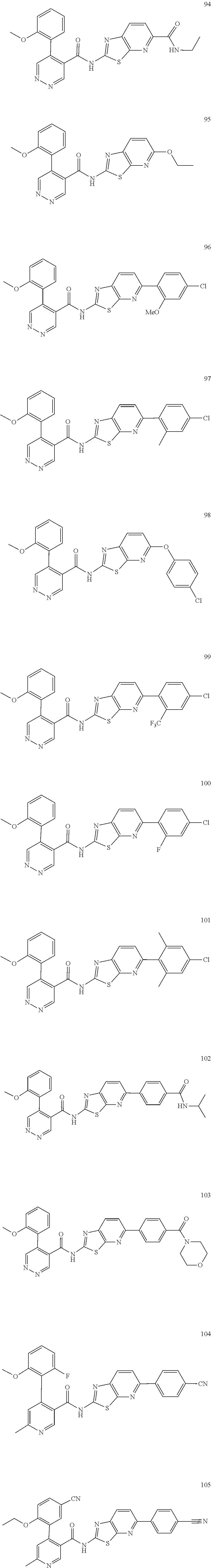 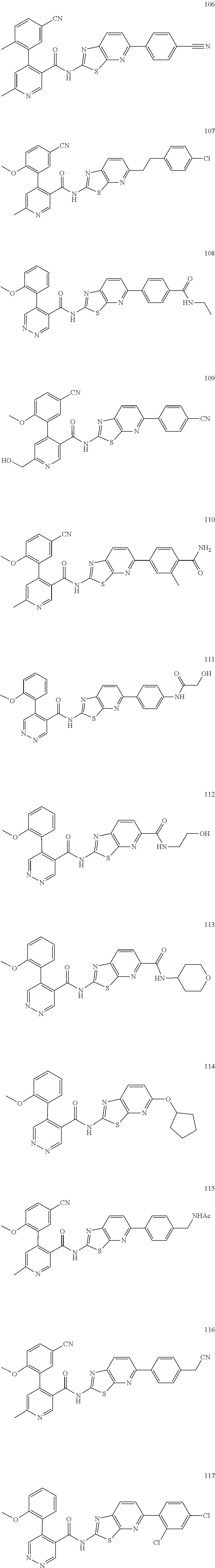 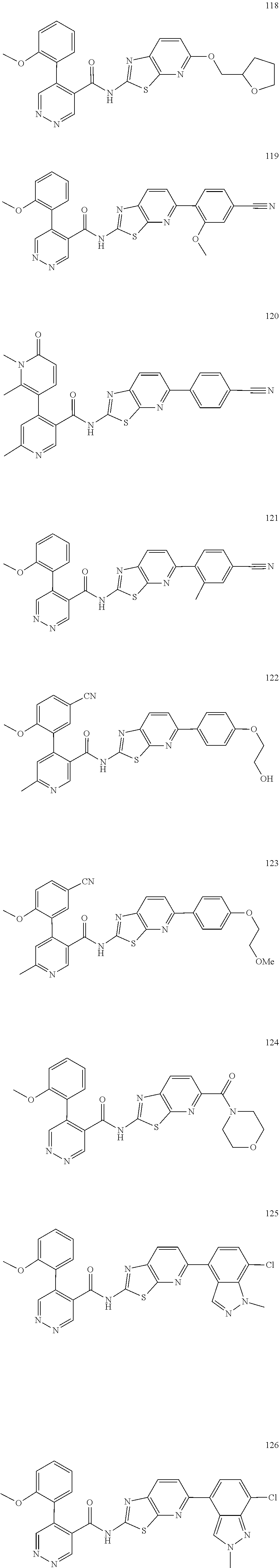 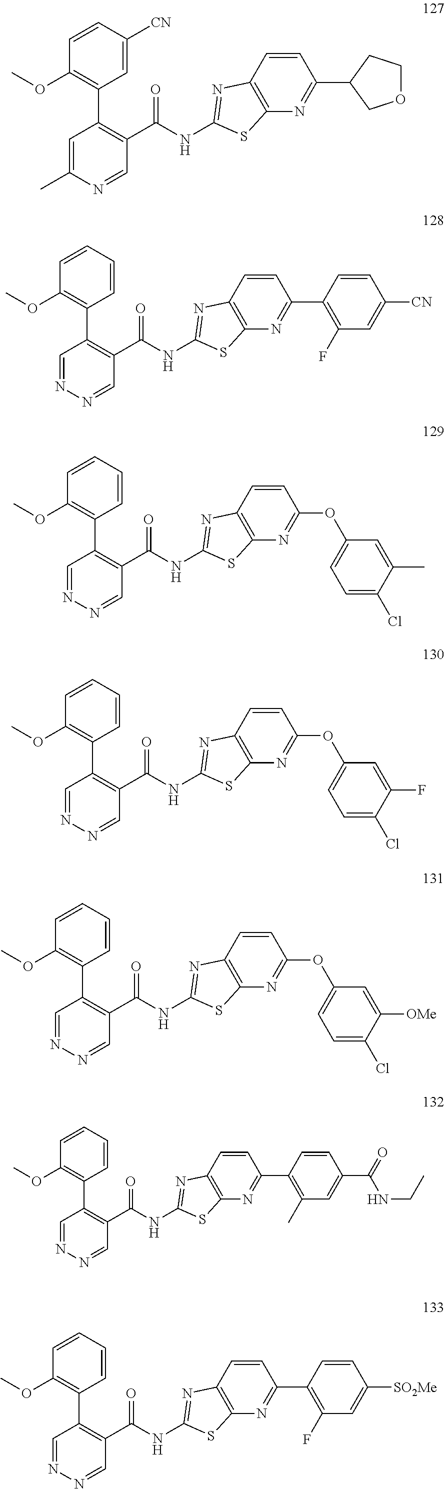 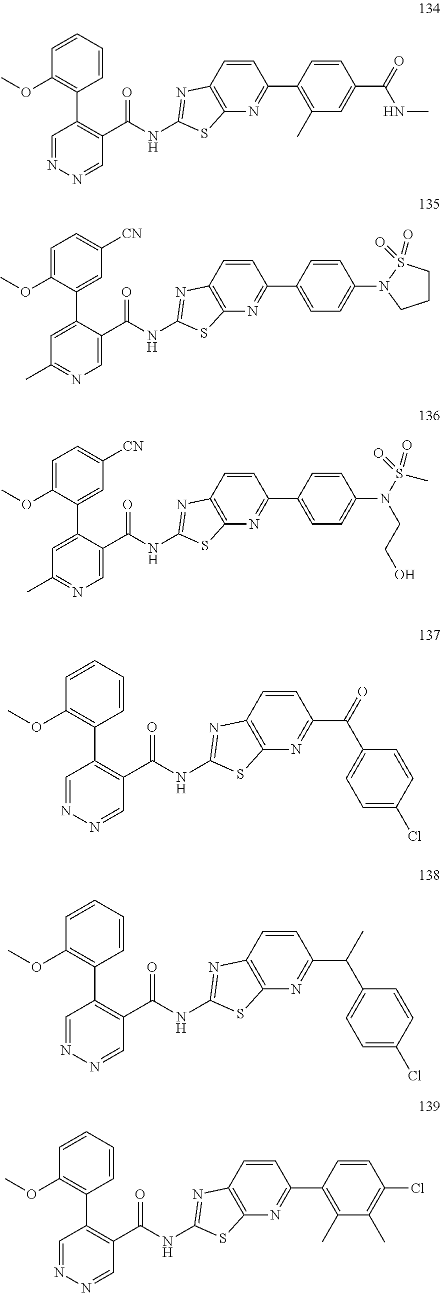 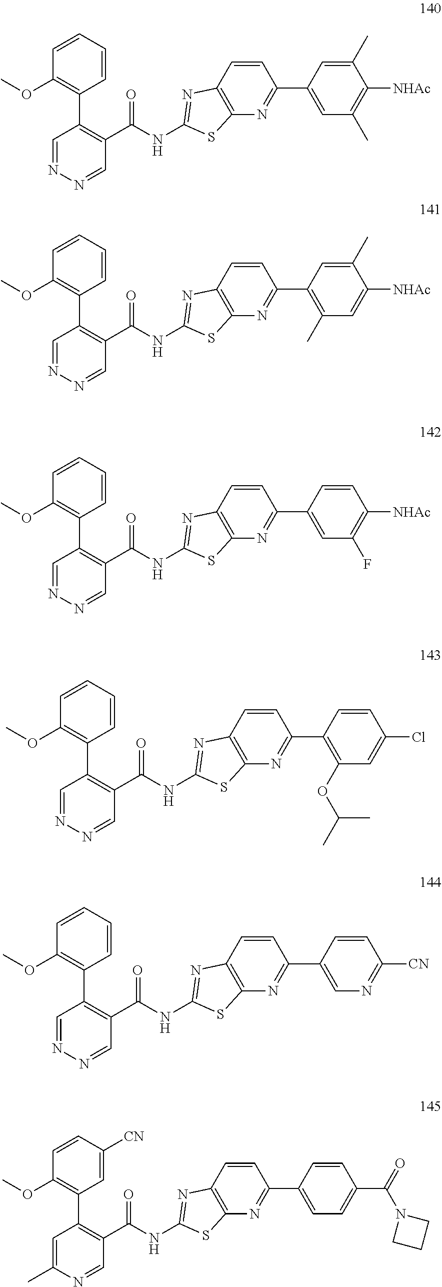 [SMILES: COc1ccccc1-c1cnncc1C(=O)Nc1nc2ccc(-c3cc(C)c(NC(C)=O)c(C)c3)nc2s1 ; COc1ccccc1-c1cnncc1C(=O)Nc1nc2ccc(-c3cc(C)c(NC(C)=O)cc3C)nc2s1 ; COc1ccccc1-c1cnncc1C(=O)Nc1nc2ccc(-c3ccc(NC(C)=O)c(F)c3)nc2s1 ; COc1ccccc1-c1cnncc1C(=O)Nc1nc2ccc(-c3ccc(Cl)cc3OC(C)C)nc2s1 ; COc1ccccc1-c1cnncc1C(=O)Nc1nc2ccc(-c3ccc(C#N)nc3)nc2s1 ; COc1ccc(C#N)cc1-c1cc(C)ncc1C(=O)Nc1nc2ccc(-c3ccc(C(=O)N4CCC4)cc3)nc2s1] 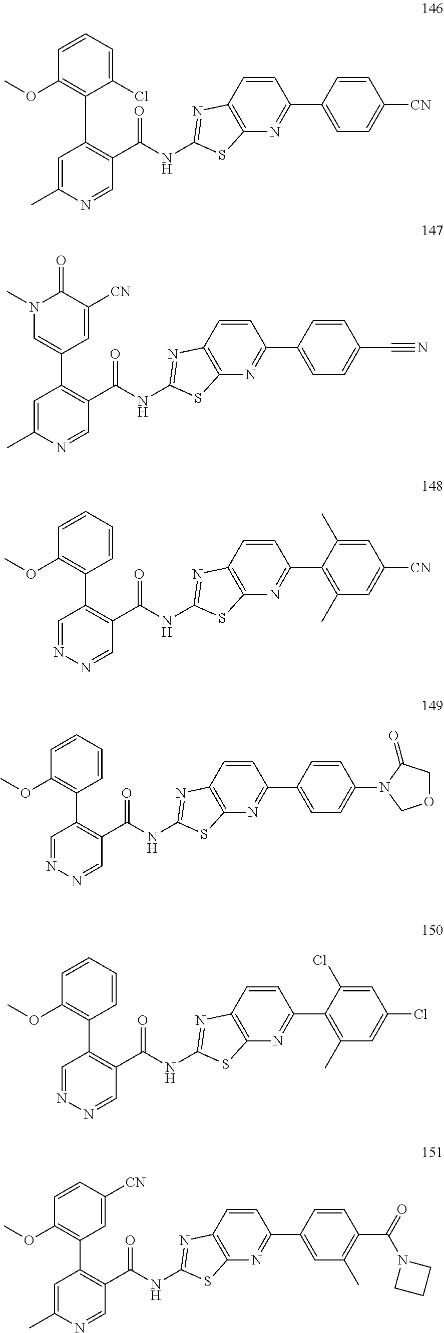 [SMILES: COc1cccc(Cl)c1-c1cc(C)ncc1C(=O)Nc1nc2ccc(-c3ccc(C#N)cc3)nc2s1 ; Cc1cc(-c2cc(C#N)c(=O)n(C)c2)c(C(=O)Nc2nc3ccc(-c4ccc(C#N)cc4)nc3s2)cn1 ; COc1ccccc1-c1cnncc1C(=O)Nc1nc2ccc(-c3c(C)cc(C#N)cc3C)nc2s1 ; COc1ccccc1-c1cnncc1C(=O)Nc1nc2ccc(-c3ccc(N4COCC4=O)cc3)nc2s1 ; COc1ccccc1-c1cnncc1C(=O)Nc1nc2ccc(-c3c(C)cc(Cl)cc3Cl)nc2s1 ; COc1ccc(C#N)cc1-c1cc(C)ncc1C(=O)Nc1nc2ccc(-c3ccc(C(=O)N4CCC4)c(C)c3)nc2s1] 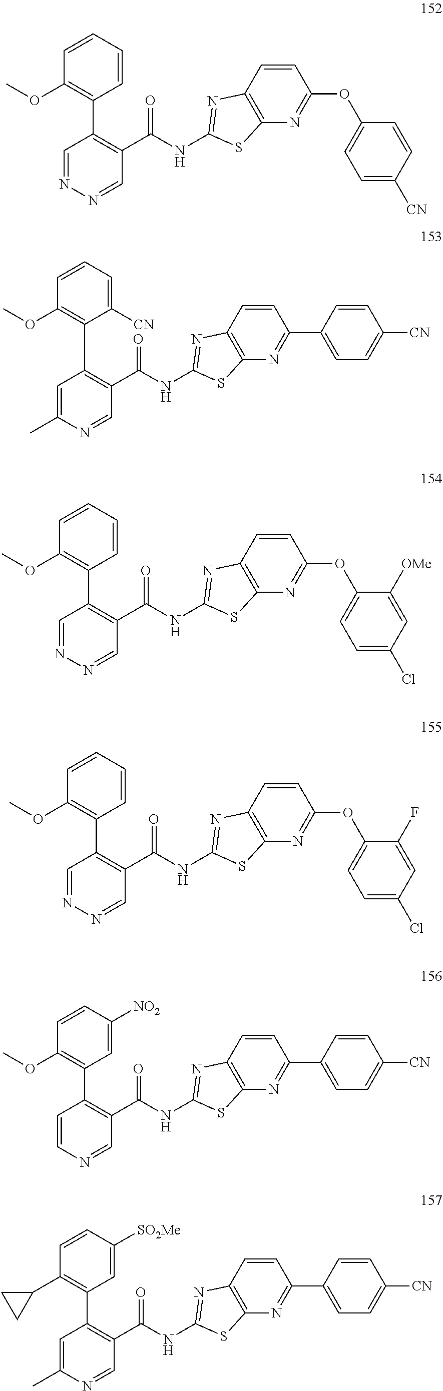 [SMILES: COc1ccccc1-c1cnncc1C(=O)Nc1nc2ccc(Oc3ccc(C#N)cc3)nc2s1 ; COc1cccc(C#N)c1-c1cc(C)ncc1C(=O)Nc1nc2ccc(-c3ccc(C#N)cc3)nc2s1 ; COc1cc(Cl)ccc1Oc1ccc2nc(NC(=O)c3cnncc3-c3ccccc3OC)sc2n1 ; COc1ccccc1-c1cnncc1C(=O)Nc1nc2ccc(Oc3ccc(Cl)cc3F)nc2s1 ; COc1ccc([N+](=O)[O-])cc1-c1ccncc1C(=O)Nc1nc2ccc(-c3ccc(C#N)cc3)nc2s1 ; Cc1cc(-c2cc(S(C)(=O)=O)ccc2C2CC2)c(C(=O)Nc2nc3ccc(-c4ccc(C#N)cc4)nc3s2)cn1] 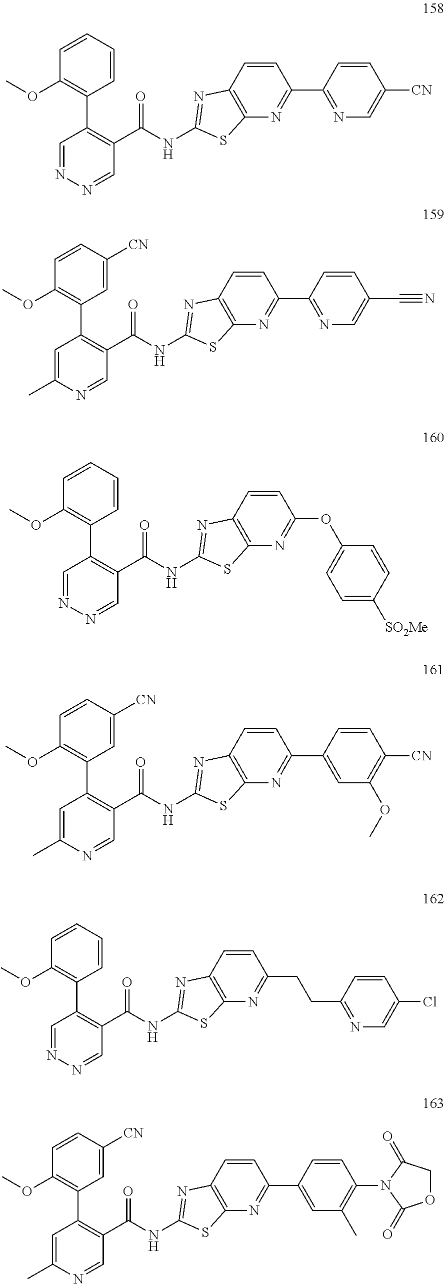 [SMILES: COc1ccccc1-c1cnncc1C(=O)Nc1nc2ccc(-c3ccc(C#N)cn3)nc2s1 ; COc1ccc(C#N)cc1-c1cc(C)ncc1C(=O)Nc1nc2ccc(-c3ccc(C#N)cn3)nc2s1 ; COc1ccccc1-c1cnncc1C(=O)Nc1nc2ccc(Oc3ccc(S(C)(=O)=O)cc3)nc2s1 ; COc1cc(-c2ccc3nc(NC(=O)c4cnc(C)cc4-c4cc(C#N)ccc4OC)sc3n2)ccc1C#N ; COc1ccccc1-c1cnncc1C(=O)Nc1nc2ccc(CCc3ccc(Cl)cn3)nc2s1 ; COc1ccc(C#N)cc1-c1cc(C)ncc1C(=O)Nc1nc2ccc(-c3ccc(N4C(=O)COC4=O)c(C)c3)nc2s1] 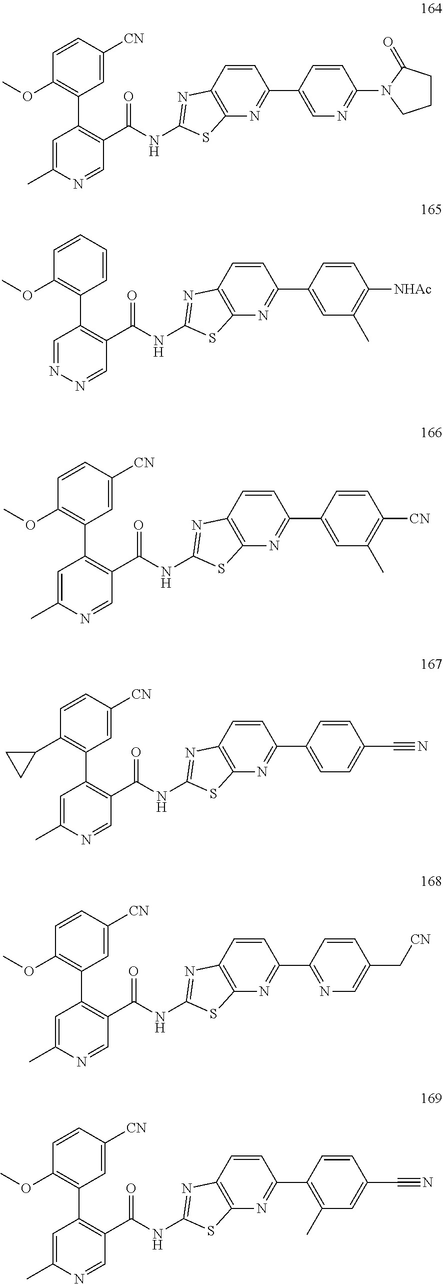 [SMILES: COc1ccc(C#N)cc1-c1cc(C)ncc1C(=O)Nc1nc2ccc(-c3ccc(N4CCCC4=O)nc3)nc2s1 ; COc1ccccc1-c1cnncc1C(=O)Nc1nc2ccc(-c3ccc(NC(C)=O)c(C)c3)nc2s1 ; COc1ccc(C#N)cc1-c1cc(C)ncc1C(=O)Nc1nc2ccc(-c3ccc(C#N)c(C)c3)nc2s1 ; Cc1cc(-c2cc(C#N)ccc2C2CC2)c(C(=O)Nc2nc3ccc(-c4ccc(C#N)cc4)nc3s2)cn1 ; COc1ccc(C#N)cc1-c1cc(C)ncc1C(=O)Nc1nc2ccc(-c3ccc(CC#N)cn3)nc2s1 ; COc1ccc(C#N)cc1-c1cc(C)ncc1C(=O)Nc1nc2ccc(-c3ccc(C#N)cc3C)nc2s1] 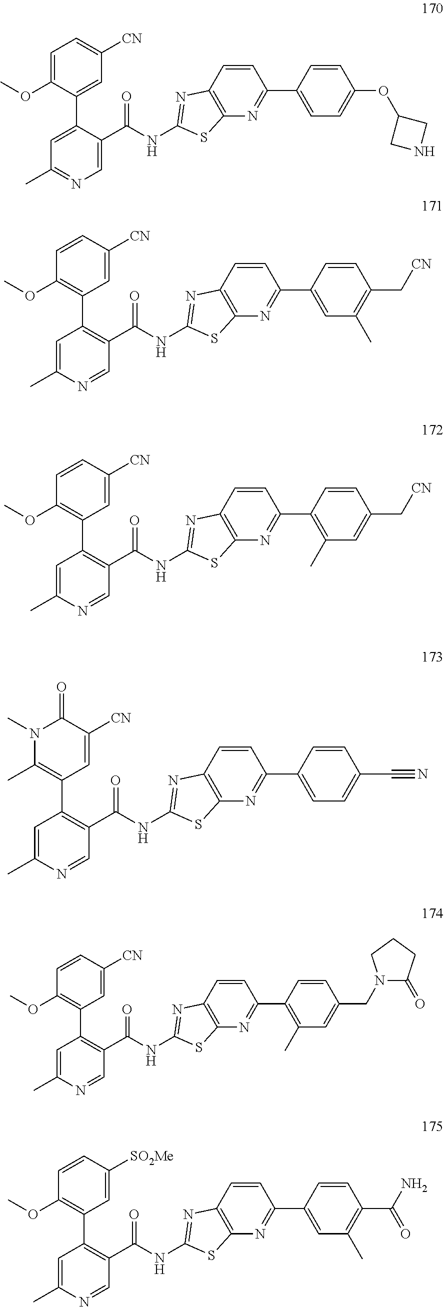 [SMILES: COc1ccc(C#N)cc1-c1cc(C)ncc1C(=O)Nc1nc2ccc(-c3ccc(OC4CNC4)cc3)nc2s1 ; COc1ccc(C#N)cc1-c1cc(C)ncc1C(=O)Nc1nc2ccc(-c3ccc(CC#N)c(C)c3)nc2s1 ; COc1ccc(C#N)cc1-c1cc(C)ncc1C(=O)Nc1nc2ccc(-c3ccc(CC#N)cc3C)nc2s1 ; Cc1cc(-c2cc(C#N)c(=O)n(C)c2C)c(C(=O)Nc2nc3ccc(-c4ccc(C#N)cc4)nc3s2)cn1 ; COc1ccc(C#N)cc1-c1cc(C)ncc1C(=O)Nc1nc2ccc(-c3ccc(CN4CCCC4=O)cc3C)nc2s1 ; COc1ccc(S(C)(=O)=O)cc1-c1cc(C)ncc1C(=O)Nc1nc2ccc(-c3ccc(C(N)=O)c(C)c3)nc2s1] 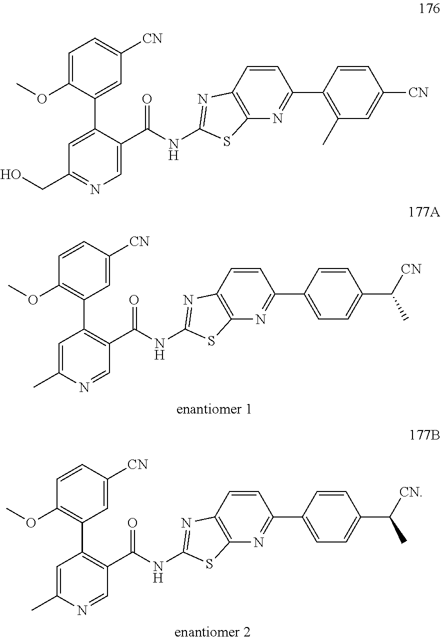 [SMILES: COc1ccc(C#N)cc1-c1cc(CO)ncc1C(=O)Nc1nc2ccc(-c3ccc(C#N)cc3C)nc2s1 ; COc1ccc(C#N)cc1-c1cc(C)ncc1C(=O)Nc1nc2ccc(-c3ccc([C@@H](C)C#N)cc3)nc2s1 ; COc1ccc(C#N)cc1-c1cc(C)ncc1C(=O)Nc1nc2ccc(-c3ccc([C@H](C)C#N)cc3)nc2s1]
or a pharmaceutically acceptable salt thereof.

27. A pharmaceutical composition comprising the compound of claim 1, or a pharmaceutically acceptable salt or stereoisomer thereof; and at least one pharmaceutically acceptable carrier or excipient.

28. A method of inhibiting Pol Theta ATPase activity, which comprises administering to an individual the compound of claim 1, or a pharmaceutically acceptable salt or stereoisomer thereof.

29. A method of treating a disease or disorder in a subject comprising administering to the subject in need thereof a therapeutically effective amount of the compound of claim 1, or a pharmaceutically acceptable salt or stereoisomer thereof.

30. The compound of claim 1, wherein the compound is selected from 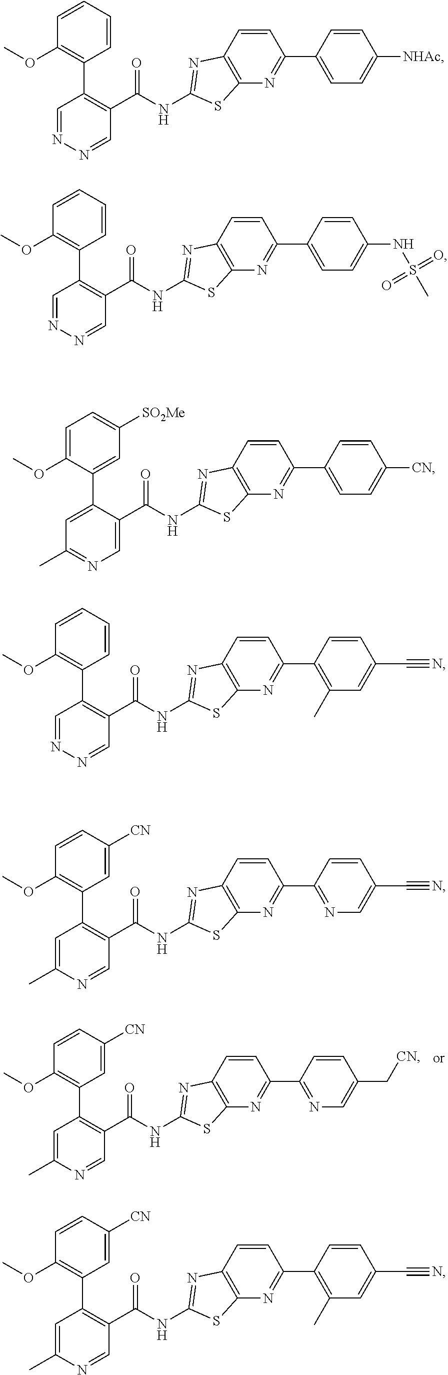
or a pharmaceutically acceptable salt thereof.
